(19) United States
(12) Patent Application Publication
(10) Pub. No.: US 2024/0254444 A1
(43) Pub. Date: Aug. 1, 2024

(54) ENGINEERED CELLS FOR THERAPY

(71) Applicant: SHORELINE BIOSCIENCES, INC., San Diego, CA (US)

(72) Inventors: John Anthony ZURIS, Boston, MA (US); Carrie Marie MARGULIES, Waban, MA (US)

(73) Assignee: SHORELINE BIOSCIENCES, INC., San Diego, CA (US)

(21) Appl. No.: 18/289,461

(22) PCT Filed: May 4, 2022
(86) PCT No.: PCT/US2022/027685

§ 371 Date: Nov. 3, 2023

Related U.S. Application Data
(60) Provisional application No. 63/321,890, filed on Mar. 21, 2022.
(60) Provisional application No. 63/297,518, filed on Jan. 7, 2022.
(60) Provisional application No. 63/275,269, filed on Nov. 3, 2021.
(60) Provisional application No. 63/270,895, filed on Oct. 22, 2021.
(60) Provisional application No. 63/233,701, filed on Aug. 16, 2021.
(60) Provisional application No. 63/233,690, filed on Aug. 16, 2021.
(60) Provisional application No. 63/233,688, filed on Aug. 16, 2021.
(60) Provisional application No. 63/228,645, filed on Aug. 3, 2021.
(60) Provisional application No. 63/184,453, filed on May 5, 2021.
(60) Provisional application No. 63/184,202, filed on May 4, 2021.

(51) Int. Cl. C12N 5/0783; A61K 39/00; A61K 39/395; A61P 35/00; C12N 9/22; C12N 15/11; C12N 15/90

(52) U.S. Cl. CPC C12N 5/0646; A61K 39/39558; A61K 39/4613; A61K 39/464499; A61P 35/00; C12N 9/22; C12N 15/11; C12N 15/907; C12N 2310/20; C12N 2506/45; C12N 2510/00; C12N 2800/80

(57) ABSTRACT

Strategies, systems, compositions, and methods for genetically modifying cells to include one or more loss-of-function modifications and/or to include one or more gain-of-function modifications, as well as modified cells (and compositions of such cells) that include one or more loss-of-function modifications and/or that include one or more gain-of-function modifications, are described. In certain aspects, such modified cells include at least one gain-of-function modification within a coding region of an essential gene.

### CROSS REFERENCE TO RELATED APPLICATIONS

This application claims the benefit of U.S. Provisional Applications Nos. 63/184,202 filed May 4, 2021, 63/184,453 filed May 5, 2021, 63/228,645 filed Aug. 3, 2021, 63/233,701 filed Aug. 16, 2021, 63/233,690 filed Aug. 16, 2021, 63/233,688 filed Aug. 16, 2021, 63/270,895 filed Oct. 22, 2021, 63/275,269 filed Nov. 3, 2021, 63/297,518 filed Jan. 7, 2022, and 63/321,890 filed Mar. 21, 2022. The entirety of each of the priority applications is incorporated herein by reference.

### BACKGROUND

Various therapeutic approaches for treatment of cancer exist, such as the use of genetically engineered cell therapies. However, engineered cells can exhibit limited tumor cell killing and/or limited persistence. There remains a need for engineered cell therapies for effective treatment of cancer.

### SUMMARY

Some aspects of the present disclosure are based, at least in part, on methods and systems for genetically modifying NK cells and/or pluripotent stem cells (e.g., iPSCs) that are, e.g., differentiated into modified iNK cells, to include one or more gain-of-function modifications (e.g., one or more gain-of-function modifications described herein), and optionally to include one or more loss-of-function modifications (e.g., one or more loss-of-function modifications described herein), as well as modified NK cells and/or modified pluripotent stem cells (e.g., iPSCs) that are, e.g., differentiated into modified iNK cells (and compositions of such cells) that include one or more gain-of-function modifications (e.g., one or more gain-of-function modifications described herein), and optionally that include one or more loss-of-function modifications (e.g., one or more loss-of-function modifications described herein). In certain aspects of the disclosure, such modified NK cells and/or modified pluripotent stem cells (e.g., iPSCs) that are, e.g., differentiated into modified iNK cells, include at least one gain-of-function modification within a coding region of an essential gene (e.g., an essential gene described herein).

In one aspect, the disclosure features a Natural Killer (NK) cell (or a progeny or daughter cell of such NK cell, or a population of such NK cells) comprising: (a) one or more genomic edits that results in loss of function of one or more of gene products; and/or (b) a genome comprising an exogenous coding sequence, wherein the exogenous coding sequence is in frame with and downstream (3′) of a coding sequence of an essential gene, and wherein at least part of the essential gene comprises an exogenous coding sequence.

In some embodiments, the one or more genomic edits results in loss of function of one or more of: adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

In some embodiments, the exogenous coding sequence encodes (i) FcγRIII (CD16) or variant thereof and/or (ii) a membrane bound interleukin 15 (mbIL-15).

In some embodiments, the genome comprises a first exogenous coding sequence and a second exogenous coding sequence. In some embodiments, the first exogenous coding sequence encodes FcγRIII (CD16) or variant thereof. In some embodiments, the second exogenous coding sequence encodes mbIL-15. In some embodiments, the first exogenous coding sequence encodes FcγRIII (CD16) or variant thereof and the second exogenous coding sequence encodes mbIL-15.

In some embodiments, the genome comprises: (i) the first exogenous coding sequence and the second exogenous coding sequence at a first allele of the essential gene; and (ii) the first exogenous coding sequence and the second exogenous coding sequence at a second allele of the essential gene.

In some embodiments, the first exogenous coding sequence is upstream (5′) of the second exogenous coding sequence. In some embodiments, the genome comprises: (i) a first regulatory element between the coding sequence of the essential gene and the first exogenous coding sequence; and (ii) a second regulatory element between the first exogenous coding sequence and the second exogenous coding sequence. In some embodiments, the first regulatory element is an IRES or 2A element and the second regulatory element is an IRES or 2A element. In some embodiments, the genome comprises a polyadenylation sequence downstream (3′) of the second exogenous coding sequence. In some embodiments, the genome comprises a 3′ untranslated region (UTR) sequence downstream (3′) of the second exogenous coding sequence and upstream (5′) of the polyadenylation sequence.

In some embodiments, the second exogenous coding sequence is upstream (5′) of the first exogenous coding sequence. In some embodiments, the genome comprises: (i) a first regulatory element between the coding sequence of the essential gene and the second exogenous coding sequence; and (ii) a second regulatory element between the second exogenous coding sequence and the first exogenous coding sequence. In some embodiments, the first regulatory element is an IRES or 2A element and the second regulatory element is an IRES or 2A element. In some embodiments, the genome comprises a polyadenylation sequence downstream (3′) of the first exogenous coding sequence. In some embodiments, the genome comprises a 3′ untranslated region (UTR) sequence downstream (3′) of the first exogenous coding sequence and upstream (5′) of the polyadenylation sequence.

In some embodiments, the first exogenous coding sequence is or comprises SEQ ID NO: 166. In some embodiments, the second exogenous coding sequence is or comprises SEQ ID NO: 172. In some embodiments, the CD16 is or comprises the amino acid sequence of SEQ ID NO: 184. In some embodiments, the mbIL-15 comprises an IL-15, a linker, a sushi domain, and an IL-15Rα. In some embodiments, the mbIL-15 is or comprises the amino acid sequence of SEQ ID NO: 190.

In some embodiments, the NK cell is an induced pluripotent stem cell (iPSC)-derived NK (INK) cell.

In some embodiments, the essential gene encodes a gene product that is required for survival and/or proliferation of the cell. In some embodiments, the essential gene is a housekeeping gene, e.g., a gene listed in Table 3. In some embodiments, the essential gene encodes glyceraldehyde 3-phosphate dehydrogenase (GAPDH).

In some embodiments, the NK cell comprises: (i) a genomic edit that results in loss of function of CISH; and (ii) a genomic edit that results in loss of function of TGFβRII.

In some embodiments, the NK cell is for use as a medicament. In some embodiments, the NK cell is for use in the treatment of a disease, disorder, or condition, e.g., a tumor and/or a cancer.

In some embodiments, the NK cell or population of NK cells is characterized in that, when contacted with tumor cells, a level of killing of tumor cells by the NK cells is increased (e.g., by at least about 10%, 20%, 40%, 60%, 80%, 100%, 150%, 200%, 300%, or more) relative to a reference level of killing of tumor cells by a reference population of NK cells, e.g., as measured using any known method, e.g., a method described in Example 11 or Example 15.

In some embodiments, the NK cell or population of NK cells is characterized in that, when contacted with tumor cells and an antibody, a level of antibody-dependent cellular cytotoxicity (ADCC) induced by the NK cells is increased (e.g., by at least about 10%, 20%, 40%, 60%, 80%, 100%, 150%, 200%, 300%, or more) relative to a reference level of ADCC induced by a reference population of NK cells, e.g., as measured using any known method, e.g., a method described in Example 11 or Example 15.

In some embodiments, a level of persistence of the population of NK cells is increased (e.g., by at least about 10%, 20%, 40%, 60%, 80%, 100%, 150%, 200%, 300%, or more) relative to a reference level of persistence of a reference population of NK cells, e.g., as measured using any known method, e.g., a method described in Example 14 or Example 15. In some embodiments, the level of persistence is measured following contacting with tumor cells.

In some embodiments, the reference population of NK cells does not comprise NK cells comprising a genome comprising the first exogenous coding sequence and the second exogenous coding sequence. In some embodiments, the reference population of NK cell does not comprise NK cells comprising a genomic edit that results in loss of function of TGFβRII and a genomic edit that results in loss of function of CISH. In some embodiments, the reference population of NK cells does not comprise NK cells comprising a genome comprising the first exogenous coding sequence and the second exogenous coding sequence, and does not comprise NK cells comprising a genomic edit that results in loss of function of TGFβRII and a genomic edit that results in loss of function of CISH.

In some aspects, the disclosure provides a pharmaceutical composition comprising an NK cell, the progeny or daughter cell, or a population of NK cells described herein. In some embodiments, the pharmaceutical composition comprises a pharmaceutically acceptable carrier.

In another aspect, the disclosure provides methods of treating a condition, disorder, and/or disease, comprising administering to a subject suffering therefrom an NK cell, a progeny or daughter cell, or a population of NK cells described herein, or a pharmaceutical composition described herein. In some embodiments, the subject is suffering from a tumor, e.g., a solid tumor. In some embodiments, the subject is suffering from a cancer.

In some embodiments, the NK cell, the progeny or daughter cell, or the population of NK cells is allogenic to the subject. In some embodiments, the NK cell, the progeny or daughter cell, or the population of NK cells is autologous to the subject. In some embodiments, the method further comprises administering an antibody to the subject. In some embodiments, the antibody is trastuzumab, rituximab, or cetuximab. In some embodiments, the subject is a human.

In another aspect, the disclosure features a method, comprising administering to a subject an NK cell, a progeny or daughter cell, or a population of NK cells described herein, or a pharmaceutical composition described herein. In some embodiments, the subject is suffering from a tumor, e.g., a solid tumor. In some embodiments, the subject is suffering from a cancer.

In some embodiments, the NK cell, the progeny or daughter cell, or the population of NK cells is allogenic to the subject. In some embodiments, the NK cell, the progeny or daughter cell, or the population of NK cells is autologous to the subject. In some embodiments, the method further comprises administering an antibody to the subject. In some embodiments, the antibody is trastuzumab, rituximab, or cetuximab. In some embodiments, the subject is a human.

In another aspect, the disclosure provides a method of increasing tumor killing ability of a NK cell, the method comprising: (a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and (b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof; thereby increasing a level of tumor killing activity of the NK cell (e.g., by at least about 10%, 20%, 40%, 60%, 80%, 100%, 150%, 200%, 300%, or more) relative to a reference level of tumor killing activity of a reference NK cell, e.g., as measured using any known method, e.g., a method described in Example 11 or Example 15.

In some embodiments, the reference NK cell does not comprise a genome comprising the first exogenous coding sequence and the second exogenous coding sequence. In some embodiments, the reference NK cell does not comprise a genomic edit that results in loss of function of TGFβRII and a genomic edit that results in loss of function of CISH. In some embodiments, the reference NK cell does not comprise a genome comprising the first exogenous coding sequence and the second exogenous coding sequence, and does not comprise a genomic edit that results in loss of function of TGFβRII and a genomic edit that results in loss of function of CISH.

In another aspect, the disclosure provides a method of increasing antibody-dependent cellular cytotoxicity (ADCC) induced by a NK cell, the method comprising: (a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and (b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof; thereby increasing a level of ADCC induced by the NK cell (e.g., by at least about 10%, 20%, 40%, 60%, 80%, 100%, 150%, 200%, 300%, or more) relative to a reference level of ADCC induced by a reference NK cell, e.g., as measured using any known method, e.g., a method described in Example 11 or Example 15.

In some embodiments, the reference NK cell does not comprise a genome comprising the first exogenous coding sequence and the second exogenous coding sequence. In some embodiments, the reference NK cell does not comprise a genomic edit that results in loss of function of TGFβRII and a genomic edit that results in loss of function of CISH. In some embodiments, the reference NK cell does not comprise a genome comprising the first exogenous coding sequence and the second exogenous coding sequence, and does not comprise a genomic edit that results in loss of function of TGFβRII and a genomic edit that results in loss of function of CISH.

In another aspect, the disclosure provides a method of increasing persistence of a NK cell, the method comprising: (a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and (b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof; thereby increasing a level of persistence of the NK cell (e.g., by at least about 10%, 20%, 40%, 60%, 80%, 100%, 150%, 200%, 300%, or more) relative to a reference level of persistence of a reference NK cell, e.g., as measured using any known method, e.g., a method described in Example 14 or Example 15. In some embodiments, the level of persistence is measured following contacting the NK cell with tumor cells.

In some embodiments, the reference NK cell does not comprise a genome comprising the first exogenous coding sequence and the second exogenous coding sequence. In some embodiments, the reference NK cell does not comprise a genomic edit that results in loss of function of TGFβRII and a genomic edit that results in loss of function of CISH. In some embodiments, the reference NK cell does not comprise a genome comprising the first exogenous coding sequence and the second exogenous coding sequence, and does not comprise a genomic edit that results in loss of function of TGFβRII and a genomic edit that results in loss of function of CISH.

In another aspect, the disclosure features a method of manufacturing a genetically modified NK cell, the method comprising: (a) knocking-into the genome of an NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and (b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

In some embodiments, knocking-in comprises contacting the NK cell with: (i) a nuclease that causes a break within an endogenous coding sequence of the essential gene, and (ii) a donor template that comprises a knock-in cassette comprising the first exogenous coding sequence and the second exogenous coding sequence in frame with and downstream (3′) of an exogenous coding sequence or partial coding sequence of the essential gene, wherein the knock-in cassette is integrated into the genome of the cell by homology-directed repair (HDR) of the break.

In some embodiments, the nuclease is a CRISPR/Cas nuclease and knocking-in further comprises contacting the NK cell with a guide molecule for the CRISPR/Cas nuclease.

In some embodiments, knocking-out comprises contacting the NK cell with one or more nucleases that cause a break within an endogenous coding sequence of the one or more genes. In some embodiments, the one or more nucleases are CRISPR/Cas nucleases and knocking-out further comprises contacting the NK cell with one or more guide molecules for the CRISPR/Cas nuclease.

In some embodiments, the NK cell is an induced pluripotent stem cell (iPSC)-derived NK (iNK) cell.

In some embodiments, the essential gene encodes a gene product that is required for survival and/or proliferation of the NK cell. In some embodiments, the essential gene is a housekeeping gene, e.g., a gene listed in Table 3. In some embodiments, the essential gene encodes glyceraldehyde 3-phosphate dehydrogenase (GAPDH).

In some embodiments, the method comprises knocking-out a gene encoding CISH and knocking-out a gene encoding TGFβRII.

In one aspect, the disclosure features an NK cell, a pluripotent human stem cell, or a modified iNK cell differentiated from such stem cell, wherein the cell comprises: (i) one or more genomic edits that results in loss of function of one or more of adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof; and (ii) a genome comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of a coding sequence of an essential gene, e.g., the GAPDH gene, wherein at least part of the coding sequence of the essential gene, e.g., the GAPDH gene comprises an exogenous coding sequence.

In one aspect, the disclosure features an NK cell, a pluripotent human stem cell, or a modified iNK cell differentiated from such stem cell, wherein the cell comprises: (i) a genome a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of a coding sequence of an essential gene, e.g., the GAPDH gene, wherein at least part of the coding sequence of the essential gene, e.g., the GAPDH gene, comprises an exogenous coding sequence; and wherein the cell comprises (ii) one or more genomic edits that results in loss of function of one or more of adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

In some embodiments, the cell comprises a genomic edit that results in a loss of function of an agonist of the TGF beta signaling pathway and a genomic edit that results in a loss of function of CISH.

In some embodiments, the cell comprises a genomic edit that results in a loss of function of a TGF beta receptor or a dominant-negative variant of a TGF beta receptor. In some embodiments, the TGF beta receptor is a TGF beta receptor II (TGFβRII).

In some embodiments, the cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

In some embodiments, the exogenous coding sequence of the GAPDH gene comprises about 2000, 1500, 1000, 750, 500, 400, 300, 200, 100, or 50 base pairs of the coding sequence of the GAPDH gene. In some embodiments, the exogenous coding sequence of the GAPDH gene comprises about 200 base pairs of the coding sequence of the GAPDH gene.

In some embodiments, the exogenous coding sequence of the GAPDH gene encodes a C-terminal fragment of a protein encoded by the GAPDH gene. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence of the GAPDH gene is less than 100% identical to the corresponding endogenous coding sequence of the GAPDH gene of the cell. In some embodiments, the exogenous coding sequence of the GAPDH gene has been codon optimized relative to the corresponding endogenous coding sequence of the GAPDH gene of the cell to remove a target site of a nuclease, e.g., a Cas. In some embodiments, the nuclease is a Cas (e.g., Cas9, Cas12a, Cas12b, Cas12c, Cas12e, CasX, or CasΦ (Cas12j), or variants thereof), the exogenous coding sequence of the GAPDH gene includes at least one PAM site for the Cas, and the at least one PAM site (or all PAM sites) has been codon optimized or saturated with silent and/or missense mutations.

In some embodiments, the cell's genome comprises a regulatory element that enables expression of the gene product encoded by the GAPDH gene and the first and second exogenous coding sequences as separate gene products, optionally, wherein at least one of the gene products is a protein and the regulatory element enables expression of that protein separate from the other gene product. In some embodiments, the cell's genome comprises an IRES or 2A element located between the coding sequence of the GAPDH gene and the first exogenous coding sequence, and/or between the first exogenous coding sequence and the second exogenous coding sequence.

In some embodiments, the cell's genome does not comprise a reporter gene, e.g., a fluorescent reporter gene or an antibiotic resistance gene.

In another aspect, the disclosure features an NK cell, a pluripotent human stem cell, or an iNK cell differentiated from such stem cell, comprising a genomic modification, wherein the modification comprises: (i) a genomic edit that results in loss of function of Cytokine Inducible SH2 Containing Protein (CISH) and (ii) a genomic edit that results in a loss of function of an agonist of the TGF beta signaling pathway; and (iii) an insertion of an exogenous knock-in cassette within an endogenous coding sequence of a GAPDH gene in the cell's genome, wherein the knock-in cassette comprises a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence encoding GAPDH, or a functional variant thereof, wherein the cell expresses FcγRIII (CD16) or variant thereof, mbIL-15, and GAPDH, or a functional variant thereof, optionally wherein FcγRIII (CD16) or variant thereof, mbIL-15, and GAPDH are expressed from the endogenous GAPDH promoter.

In another aspect, the disclosure features an NK cell, a pluripotent human stem cell, or an iNK cell differentiated from such stem cell, comprising a genomic modification, wherein the modification comprises: (i) an insertion of an exogenous knock-in cassette within an endogenous coding sequence of a GAPDH gene in the cell's genome, wherein the knock-in cassette comprises a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence encoding GAPDH, or a functional variant thereof, wherein the cell expresses FcγRIII (CD16) or variant thereof, mbIL-15, and GAPDH, or a functional variant thereof, optionally wherein FcγRIII (CD16) or variant thereof, mbIL-15, and GAPDH are expressed from the endogenous GAPDH promoter, and wherein the NK cell, pluripotent human stem cell, or iNK cell differentiated from such a stem cell further comprises (ii) one or more genomic edits that results in loss of function of one or more of adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

In some embodiments, the cell comprises a genomic edit that results in a loss of function of an agonist of the TGF beta signaling pathway and a genomic edit that results in a loss of function of CISH.

In some embodiments, the cell comprises a genomic edit that results in a loss of function of a TGF beta receptor or a dominant-negative variant of a TGF beta receptor. In some embodiments, the TGF beta receptor is a TGF beta receptor II (TGFβRII).

In some embodiments, the cell expresses one or more pluripotency markers selected from the group consisting of SSEA-3, SSEA-4, TRA-1-60, TRA-1-81, TRA-2-49/6E, ALP, Sox2, E-cadherin, UTF-1, Oct4, Rex1, and Nanog.

In some embodiments, the exogenous coding sequence or partial coding sequence encoding GAPDH comprises about 2000, 1500, 1000, 750, 500, 400, 300, 200, 100, or 50 base pairs of the coding sequence of the GAPDH gene. In some embodiments, the exogenous coding sequence or partial coding sequence encoding GAPDH comprises about 200 base pairs of the coding sequence of the GAPDH gene.

In some embodiments, the exogenous coding sequence or partial coding sequence encoding GAPDH encodes a C-terminal fragment of GAPDH. In some embodiments, the C-terminal fragment is less than about 500, 250, 150, 125, 100, 75, 50, 25, 20, 15 or 10 amino acids in length. In some embodiments, the C-terminal fragment is less than about 25 amino acids in length. In some embodiments, the C-terminal fragment includes an amino acid sequence that is encoded by a region of the endogenous coding sequence of the GAPDH gene that spans the break.

In some embodiments, the exogenous coding sequence or partial coding sequence encoding GAPDH is less than 100% identical to the corresponding endogenous coding sequence of the GAPDH gene of the cell. In some embodiments, the exogenous coding sequence or partial coding sequence encoding GAPDH has been codon optimized relative to the corresponding endogenous coding sequence of the GAPDH gene of the cell to remove a target site of a nuclease, e.g., a Cas. In some embodiments, the nuclease is a Cas (e.g., Cas9, Cas12a, Cas12b, Cas12c, Cas12e, CasX, CasΦ (Cas12j)), or a variant thereof), the exogenous coding sequence or partial coding sequence encoding GAPDH includes at least one PAM site for the Cas, and the at least one PAM site (or all PAM sites) has been codon optimized or saturated with silent and/or missense mutations.

In some embodiments, the cell's genome comprises a regulatory element that enables expression of the gene product encoded by the GAPDH gene and the first and second exogenous coding sequences as separate gene products, optionally, wherein at least one of the gene products is a protein and the regulatory element enables expression of that protein separate from the other gene product. In some embodiments, the cell's genome comprises an IRES or 2A element located between the coding sequence of the GAPDH gene and the first exogenous coding sequence and/or between the first exogenous coding sequence and the second exogenous coding sequence.

In some embodiments, the first exogenous coding sequence is upstream (5′) of the second exogenous coding sequence, and the cell's genome comprises a polyadenylation sequence, and optionally a 3′ UTR sequence, downstream of the second exogenous coding sequence, and, if a 3′UTR sequence is present, the 3′UTR sequence is positioned 3′ of the second exogenous coding sequence and 5′ of the polyadenylation sequence.

In some embodiments, the second exogenous coding sequence is upstream (5′) of the first exogenous coding sequence, and the cell's genome comprises a polyadenylation sequence, and optionally a 3′ UTR sequence, downstream of the first exogenous coding sequence, and, if a 3′UTR sequence is present, the 3′UTR sequence is positioned 3′ of the first exogenous coding sequence and 5′ of the polyadenylation sequence.

In some embodiments, the cell's genome does not comprise a reporter gene, e.g., a fluorescent reporter gene or an antibiotic resistance gene.

In some embodiments, the knock-in cassette comprises the first exogenous coding sequence, a linker (e.g., T2A, P2A, and/or IRES), and the second exogenous coding sequence. In some embodiments, the genome-edited cell comprises (i) knock-in cassettes at one or both alleles of the GAPDH gene; and (ii) one or more loss-of-function modifications at one or both alleles. In some embodiments, the genome-edited cell expresses FcγRIII (CD16) or variant thereof, mbIL-15, and GAPDH, or a functional variant thereof.

In some embodiments, the engineered cell comprises (i) one or more loss-of-function modifications at one or both alleles (e.g., at least one genomic edit that results in a loss of function of at least one of: CISH; TGF beta signaling pathway; ADORA2A; T cell immunoreceptor with Ig and ITIM domains (TIGIT); β-2 microglobulin (B2M); programmed cell death protein 1 (PD-1); class II, major histocompatibility complex, transactivator (CIITA); natural killer cell receptor NKG2A (natural killer group 2A); two or more HLA class II histocompatibility antigen alpha chain genes, and/or two or more HLA class II histocompatibility antigen beta chain genes; cluster of differentiation 32B (CD32B, FCGR2B); T cell receptor alpha constant (TRAC); or any combination of two or more thereof) and (ii) multi-cistronic knock-ins (e.g., at one or both alleles of GAPDH gene) of coding sequence for FcγRIII (CD16) or variant thereof and coding sequence for mbIL-15.

In some embodiments, the engineered cell comprises (i) one or more loss-of-function modifications at one or both alleles (e.g., at least one genomic edit that results in a loss of function of at least one of: CISH; TGF beta signaling pathway; ADORA2A; T cell immunoreceptor with Ig and ITIM domains (TIGIT); β-2 microglobulin (B2M); programmed cell death protein 1 (PD-1); class II, major histocompatibility complex, transactivator (CIITA); natural killer cell receptor NKG2A (natural killer group 2A); two or more HLA class II histocompatibility antigen alpha chain genes, and/or two or more HLA class II histocompatibility antigen beta chain genes; cluster of differentiation 32B (CD32B, FCGR2B); T cell receptor alpha constant (TRAC); or any combination of two or more thereof); and (ii) bi-allelic knock-ins (e.g., the first exogenous coding sequence at a first allele of GAPDH gene, and the second exogenous coding sequence at a second allele of GAPDH gene).

In some embodiments, the disclosure features a differentiated iNK cell, wherein the differentiated iNK cell is a daughter cell of a pluripotent human stem cell described herein. In some embodiments, the cell does not express endogenous CD3, CD4, and/or CD8.

In some embodiments, a genomic edit resulting in loss of function of CISH in any of the cells described herein was produced using a guide RNA comprising a targeting domain sequence comprising or consisting of the nucleotide sequence according to any one of SEQ ID NO: 258-364, 1155, 1162, and 1173. In some embodiments, a genomic edit resulting in loss of function of CISH in any of the cells described herein was produced using a guide RNA comprising a targeting domain sequence comprising or consisting of a nucleotide sequence that is identical to, or differs by no more than 1, 2, or 3 nucleotides from, any one of SEQ ID NO: 258-364, 1155, 1162, and 1173. In some embodiments, a genomic edit resulting in loss of function of CISH in any of the cells described herein was produced using a guide RNA comprising (i) a targeting domain sequence comprising or consisting of the nucleotide sequence of SEQ ID NO:1155 or 1162, and (ii) a 5′ extension sequence depicted in Table 6. In some embodiments, a genomic edit resulting in loss of function of CISH in any of the cells described herein was produced using a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence.

In some embodiments, a genomic edit resulting in loss of function of TGFβRII in any of the cells described herein was produced using a guide RNA comprising a targeting domain sequence comprising or consisting of the nucleotide sequence according to any one of SEQ ID NO: 29-257, 1157, 1161, and 1172. In some embodiments, a genomic edit resulting in loss of function of TGFβRII in any of the cells described herein was produced using a guide RNA comprising a targeting domain sequence comprising or consisting of a nucleotide sequence that is identical to, or differs by no more than 1, 2, or 3 nucleotides from, any one of SEQ ID NO: 29-257, 1157, 1161, and 1172. In some embodiments, a genomic edit resulting in loss of function of TGFβRII in any of the cells described herein was produced using a guide RNA comprising (i) a targeting domain sequence comprising or consisting of the nucleotide sequence of SEQ ID NO: 1157 or 1161, and (ii) a 5′ extension sequence depicted in Table 6. In some embodiments, a genomic edit resulting in loss of function of TGFβRII in any of the cells described herein was produced using a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence.

In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising a targeting domain sequence comprising or consisting of the nucleotide sequence according to any one of SEQ ID NO: 258-364, 1155, 1162, and 1173. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising a targeting domain sequence comprising or consisting of a nucleotide sequence that is identical to, or differs by no more than 1, 2, or 3 nucleotides from, any one of SEQ ID NO: 258-364, 1155, 1162, and 1173. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising (i) a targeting domain sequence comprising or consisting of the nucleotide sequence of SEQ ID NO:1155 or 1162, and (ii) a 5′ extension sequence depicted in Table 6. In some embodiments, a genomic edit resulting in loss of function of CISH in any of the cells described herein was produced using a ribonucleoprotein (RNP) complex comprising (i) an RNA-guided nuclease (e.g., a Cas12a variant, e.g., a Cas 12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62), and (ii) a guide RNA comprising a targeting domain sequence comprising or consisting of the nucleotide sequence according to any one of SEQ ID NO: 29-257, 1157, 1161, and 1172. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising a targeting domain sequence comprising or consisting of a nucleotide sequence that is identical to, or differs by no more than 1, 2, or 3 nucleotides from, any one of SEQ ID NO: 29-257, 1157, 1161, and 1172. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas 12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62), and (ii) a guide RNA comprising (i) a targeting domain sequence comprising or consisting of the nucleotide sequence of SEQ ID NO: 1157 or 1161, and (ii) a 5′ extension sequence depicted in Table 6. In some embodiments, a genomic edit resulting in loss of function of TGFβRII in any of the cells described herein was produced using a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62), and (ii) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence.

In another aspect, the disclosure features a method of making a cell, e.g., a cell described herein, the method comprising (A) contacting an NK cell, a pluripotent human stem cell or human induced pluripotent stem cell, with: an RNA-guided nuclease and a guide RNA comprising a targeting domain sequence comprising or consisting of a nucleotide sequence that is identical to, or differs by no more than 1, 2, or 3 nucleotides from, any one of 258-364, 1155, 1162, and 1173; and an RNA-guided nuclease and a guide RNA comprising a targeting domain sequence comprising or consisting of a nucleotide sequence that is identical to, or differs by no more than 1, 2, or 3 nucleotides from, any one of 29-257, 1157, 1161, and 1172; and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In some embodiments, the method comprises contacting the cell with: (1) a guide RNA comprising (i) a targeting domain sequence comprising or consisting of the nucleotide sequence of SEQ ID NO:1155 or 1162, and (ii) a 5′ extension sequence depicted in Table 6; and (2) a guide RNA comprising (i) a targeting domain sequence comprising or consisting of the nucleotide sequence of SEQ ID NO: 1157 or 1161, and (ii) a 5′ extension sequence depicted in Table 6.

In some embodiments, the method comprises contacting the cell with: (1) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence; and (2) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence.

In some embodiments, the RNA-guided nuclease is a Cas12a variant. In some embodiments, the Cas12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO: 62.

In another aspect, the disclosure features a method of making a cell, e.g., a cell described herein, the method comprising (A) contacting an NK cell, a pluripotent human stem cell or a human induced pluripotent stem cell, with: a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising a targeting domain sequence comprising or consisting of a nucleotide sequence that is identical to, or differs by no more than 1, 2, or 3 nucleotides from, any one of SEQ ID NO: 258-364, 1155, 1162, and 1173; and a ribonucleoprotein (RNP) complex comprising (i) an RNA-guided nuclease (e.g., a Cas12a variant, e.g., a Cas12a variant comprising 1, 2, or 3 of the amino acid substitutions selected from M537R, F870L, and H800A, e.g., a Cas12a variant comprising an amino acid sequence having 90%, 95%, or 100% identity to SEQ ID NO: 62) and (ii) a guide RNA comprising a targeting domain sequence comprising or consisting of a nucleotide sequence that is identical to, or differs by no more than 1, 2, or 3 nucleotides from, any one of SEQ ID NO: 29-257, 1157, 1161, and 1172; and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In some embodiments, the method comprises contacting the cell with: (1) an RNP comprising a guide RNA comprising (i) a targeting domain sequence comprising or consisting of the nucleotide sequence of SEQ ID NO: 1155 or 1162, and (ii) a 5′ extension sequence depicted in Table 6; and (2) an RNP comprising a guide RNA comprising (i) a targeting domain sequence comprising or consisting of the nucleotide sequence of SEQ ID NO: 1157 or 1161, and (ii) a 5′ extension sequence depicted in Table 6.

In some embodiments, the method comprises contacting the cell with: (1) an RNP comprising a guide RNA comprising (i) a targeting domain sequence comprising or consisting of the nucleotide sequence of SEQ ID NO: 1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence; and (2) an RNP comprising a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence.

In some embodiments, the RNA-guided nuclease is a Cas12a variant. In some embodiments, the Cas12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO: 62.

In another aspect, the disclosure features a method of making a cell, e.g., a cell described herein, the method comprising (A) contacting an NK cell, a pluripotent human stem cell or a human induced pluripotent stem cell, with (i) a guide RNA comprising a targeting domain sequence comprising or consisting of the nucleotide sequence of SEQ ID NO: 1155 or 1162; and a guide RNA comprises a targeting domain sequence comprising or consisting of the nucleotide sequence of SEQ ID NO: 1157 or 1161; and (ii) an RNA-guided nuclease comprising an amino acid sequence having 90%, 95%, or 100% identity to one of SEQ ID NO:58-66 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant, (b) mbIL-15, and (c) GAPDH, or a functional variant thereof.

In another aspect, the disclosure features a method of making a cell, e.g., a cell described herein, the method comprising (A) contacting an NK cell, a pluripotent human stem cell or a human induced pluripotent stem cell, with (1) an RNP comprising (i) a guide RNA comprising a targeting domain sequence comprising or consisting of the nucleotide sequence of SEQ ID NO: 1155 or 1162; and (ii) an RNA-guided nuclease comprising an amino acid sequence having 90%, 95%, or 100% identity to one of SEQ ID NO:58-66 (or a portion thereof); and (2) an RNP comprising (i) a guide RNA comprises a targeting domain sequence comprising or consisting of the nucleotide sequence of SEQ ID NO: 1157 or 1161, and (ii) an RNA-guided nuclease comprising an amino acid sequence having 90%, 95%, or 100% identity to one of SEQ ID NO:58-66 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In another aspect, the disclosure features a method of making a cell, e.g., a cell described herein, the method comprising (A) contacting an NK cell, a pluripotent human stem cell or a human induced pluripotent stem cell, with (1) a guide RNA comprising (i) a targeting domain sequence comprising or consisting of the nucleotide sequence of SEQ ID NO:1155 or 1162, and (ii) a 5′ extension sequence depicted in Table 6; (2) a guide RNA comprising (i) a targeting domain sequence comprising or consisting of the nucleotide sequence of SEQ ID NO: 1157 or 1161, and (ii) a 5′ extension sequence depicted in Table 6; and (3) an RNA-guided nuclease comprising an amino acid sequence having 90%, 95%, or 100% identity to one of SEQ ID NO:58-66 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In another aspect, the disclosure features a method of making a cell, e.g., a cell described herein, the method comprising (A) contacting an NK cell, a pluripotent human stem cell or a human induced pluripotent stem cell, with (1) an RNP comprising (a) a guide RNA comprising (i) a targeting domain sequence comprising or consisting of the nucleotide sequence of SEQ ID NO:1155 or 1162, and (ii) a 5′ extension sequence depicted in Table 6; and (b) an RNA-guided nuclease comprising an amino acid sequence having 90%, 95%, or 100% identity to one of SEQ ID NO:58-66 (or a portion thereof); and (2) an RNP comprising (a) a guide RNA comprising (i) a targeting domain sequence comprising or consisting of the nucleotide sequence of SEQ ID NO: 1157 or 1161, and (ii) a 5′ extension sequence depicted in Table 6; and (b) an RNA-guided nuclease comprising an amino acid sequence having 90%, 95%, or 100% identity to one of SEQ ID NO:58-66 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In another aspect, the disclosure features a method of making a cell, e.g., a cell described herein, the method comprising (A) contacting an NK cell, a pluripotent human stem cell or a human induced pluripotent stem cell, with (1) a guide RNA comprising (i) a targeting domain sequence comprising or consisting of the nucleotide sequence of SEQ ID NO: 1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; (2) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO:1154 at the 5′ of the scaffold sequence; and (3) an RNA-guided nuclease comprising an amino acid sequence having 90%, 95%, or 100% identity to one of SEQ ID NO:58-66 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In another aspect, the disclosure features a method of making a cell, e.g., a cell described herein, the method comprising (A) contacting an NK cell, a pluripotent human stem cell or a human induced pluripotent stem cell, with (1) an RNP comprising (a) a guide RNA comprising (i) a targeting domain sequence comprising or consisting of the nucleotide sequence of SEQ ID NO: 1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence; and (b) an RNA-guided nuclease comprising an amino acid sequence having 90%, 95%, or 100% identity to one of SEQ ID NO:1144-1151 (or a portion thereof); and (2) an RNP comprising (a) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence; and (b) an RNA-guided nuclease comprising an amino acid sequence having 90%, 95%, or 100% identity to one of SEQ ID NO:1144-1151 (or a portion thereof); and (B) contacting the cell with: (i) a nuclease that causes a break within an endogenous coding sequence of a glyceraldehyde 3-phosphate dehydrogenase (GAPDH) gene in the cell, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) GAPDH, or a functional variant thereof.

In another aspect, the disclosure features a method of making a modified cell, e.g., a modified NK cell, a modified pluripotent human stem cell, a modified NK cell differentiated from such a stem cell, the method comprising (A) contacting a cell with: (i) an RNA-guided nuclease and a guide RNA that cause a break within an endogenous coding sequence of an essential gene in the cell, such as, e.g., glyceraldehyde 3-phosphate dehydrogenase (GAPDH) gene, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the essential gene, e.g., the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) the essential gene, e.g., GAPDH, or a functional variant thereof; and, (B) contacting the cell (e.g., the NK cell or the pluripotent human stem cell or the human induced pluripotent stem cell) with one or more of: at least one RNA-guided nuclease and at least one guide RNA comprising a targeting domain sequence, wherein the RNA-guided nuclease and the guide RNA cause a genomic edit within an endogenous coding sequence of a gene of interest, e.g., a break or genomic edit resulting in a loss of function of the gene of interest, wherein the gene of interest comprises, e.g., adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

In some embodiments, the method comprises contacting the cell with: (1) a guide RNA comprising (i) a targeting domain sequence comprising or consisting of the nucleotide sequence of SEQ ID NO:1155 or 1162, and (ii) a 5′ extension sequence depicted in Table 6; and (2) a guide RNA comprising (i) a targeting domain sequence comprising or consisting of the nucleotide sequence of SEQ ID NO: 1157 or 1161, and (ii) a 5′ extension sequence depicted in Table 6.

In some embodiments, the method comprises contacting the cell with: (1) a guide RNA comprising (i) a targeting domain sequence comprising or consisting of the nucleotide sequence of SEQ ID NO:1155 or 1162, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence; and (2) a guide RNA comprising (i) a targeting domain sequence comprising or consisting of the nucleotide sequence of SEQ ID NO: 1157 or 1161, (ii) a scaffold sequence comprising or consisting of the nucleotide sequence of SEQ ID NO: 1153 located 5′ of the targeting domain sequence, and (iii) the nucleotide sequence of SEQ ID NO: 1154 at the 5′ of the scaffold sequence.

In some embodiments, the RNA-guided nuclease is a Cas12a variant. In some embodiments, the Cas12a variant comprises one or more amino acid substitutions selected from M537R, F870L, and H800A. In some embodiments, the Cas12a variant comprises amino acid substitutions M537R, F870L, and H800A. In some embodiments, the Cas12a variant comprises an amino acid sequence having 90%, 95%, or 100% identity to SEQ ID NO: 62.

In another aspect, the disclosure features a method of making a population of modified cells, e.g., a population of modified NK cells, a population of modified pluripotent human stem cells, a population of modified NK cells differentiated from such stem cells, the method comprising (A) contacting a population of cells with: (i) an RNA-guided nuclease and a guide RNA (e.g., configured together as an RNP) that cause a break within an endogenous coding sequence of an essential gene in at least one cell within the population of cells, such as, e.g., glyceraldehyde 3-phosphate dehydrogenase (GAPDH) gene, and (ii) a donor template that comprises a knock-in cassette comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of an exogenous coding sequence or partial coding sequence of the essential gene, e.g., the GAPDH gene, wherein the knock-in cassette is integrated into the genome of the cell by homology-directed repair (HDR) of the break, resulting in a genome-edited cell that expresses: (a) FcγRIII (CD16) or variant thereof, (b) mbIL-15, and (c) the essential gene, e.g., GAPDH, or a functional variant thereof; and (B) contacting the population of cells (e.g., the population of NK cells or the population of pluripotent human stem cells or the population of induced human induced pluripotent stem cells) with one or more of: at least one RNA-guided nuclease and a guide RNA comprising a targeting domain sequence, wherein the RNA-guided nuclease and the guide RNA cause a genomic edit within an endogenous coding sequence of a gene of interest within at least one cell in the population of cells, e.g., a genomic edit resulting in a break and/or a genomic edit resulting in a loss of function of the gene of interest, wherein the gene of interest comprises, e.g., adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof. In some embodiments, the population of cells is optionally contacted with at least a first RNA-guided nuclease and a first guide RNA that cause a genomic edit within the endogenous coding sequence of a first gene of interest and a second RNA-guided nuclease and a second guide RNA that cause a genomic edit within the endogenous coding sequence of a second gene of interest; and, optionally, wherein the population of cells is contacted with a third, fourth, and/or fifth (or more) RNA-guided nuclease and a third, fourth, and/or fifth (or more) guide RNA that causes a genomic edit within the endogenous coding sequence of a third, fourth, and/or fifth (or more) gene of interest, respectively.

In some embodiments, the RNA-guided nuclease editing efficiency is high, e.g., wherein the RNA-guided nuclease is capable of editing about 60% to 100% of cells in a population of cells, e.g., at least 60%, at least 65%, at least 70%, at least 75%, at least 80%, at least 85%, at least 90%, at least 95%, or at least 99% oc cells in a population. In some embodiments, the RNA-guided nuclease is configured with a guide RNA to form an RNP, and the RNP causes a break within the essential gene (e.g., within the terminal exon in the locus of any essential gene provided in Table 3, such as, e.g., GAPDH) in at least 60% of the cells in the population of cells (e.g., in at least 60%, in at least 65%, in at least 70%, in at least 75%, in at least 80%, in at least 85%, in at least 90%, in at least 91%, in at least 92%, in at least 93%, in at least 94%, in at least 95%, in at least 96%, in at least 97%, in at least 98%, or in at least 99% of the cells in the population of cells). In some embodiments, the RNA-guided nuclease is configured with a guide RNA to form an RNP, and the RNP induces knock-in cassette integration at the essential gene (e.g., within the terminal exon in the locus of any essential gene provided in Table 3, such as, e.g., GAPDH) in at least 50% of the cells in the population of cells (e.g., in at least 50%, in at least 55%, in at least 60%, in at least 65%, in at least 70%, in at least 75%, in at least 80%, in at least 85%, in at least 90%, in at least 91%, in at least 92%, in at least 93%, in at least 94%, in at least 95%, in at least 96%, in at least 97%, in at least 98%, or in at least 99% of the cells in the population of cells) at between 4 days and 9 days (e.g., at 4 days, 5 days, 6 days, 7 days, 8 days or 9 days) after the population of cells is contacted with the RNA-guided nuclease and the guide RNA (the RNP) and the donor template. In some embodiments, the RNA-guided nuclease comprises Cas9, Cas12a, Cas12b, Cas12c, Cas12e, CasX, or CasΦ (Cas12j), or a variant thereof, e.g., a variant capable of editing about 60% to 100% of cells in a population of cells.

In some embodiment, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells comprises the knock-in cassette comprising the first and second exogenous coding sequences integrated at the essential gene in the genome at between 4 days and 9 days (e.g., at 4 days, 5 days, 6 days, 7 days, 8 days or 9 days) after the population of cells is contacted with the donor template and the RNA-guided nuclease and the guide RNA (e.g., configured together an an RNP) that cause a break within the endogenous coding sequence of the essential gene.

In some embodiments, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells comprises the knock-in cassette comprising the first and second exogenous coding sequences integrated at the essential gene in the genome at between 4 days and 9 days (e.g., at 4 days, 5 days, 6 days, 7 days, 8 days or 9 days) after the population of cells is contacted with the donor template and the RNA-guided nuclease and the guide RNA (e.g., configured together an an RNP) that cause a break within the endogenous coding sequence of the essential gene, and at least 60% of the cells (e.g., at least 60%, at least 65%, at least 70%, at least 75%, at least 80%, at least 85%, at least 90%, at least 95%, at least 98%, or at least 99%) in the population of cells comprises a genomic edit (e.g., a genomic edit resulting in a break and/or a genomic edit resulting in a loss of function) within an endogenous coding sequence of a gene of interest, e.g., at between 4 days and 9 days (e.g., at 4 days, 5 days, 6 days, 7 days, 8 days or 9 days) after the population of cells is contacted with the an RNA-guided nuclease and a guide RNA (e.g., configured together an RNP) that cause a genomic edit within the endogenous coding sequence of the gene of interest.

In some embodiments, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells comprise the knock-in cassette comprising the first and second exogenous coding sequences integrated at the essential gene in the genome at between 4 days and 9 days (e.g., at 4 days, 5 days, 6 days, 7 days, 8 days or 9 days) after the population of cells is contacted with the donor template and the RNA-guided nuclease and the guide RNA (e.g., configured together as an RNP) that cause a break within the endogenous coding sequence of the essential gene, and at least 60% of the cells (e.g., at least 60%, at least 65%, at least 70%, at least 75%, at least 80%, at least 85%, at least 90%, at least 95%, at least 98%, or at least 99%) in the population of cells comprise a genomic edit (e.g., a genomic edit resulting in a break and/or a genomic edit resulting in a loss of function) within an endogenous coding sequence of a gene of interest, e.g., at between 4 days and 9 days (e.g., at 4 days, 5 days, 6 days, 7 days, 8 days or 9 days) after the population of cells is contacted with an RNA-guided nuclease and a guide RNA (e.g., configured together as an RNP) that cause a genomic edit within the endogenous coding sequence of the gene of interest. In some embodiments, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells comprise the knock-in cassette comprising the first and second exogenous coding sequences integrated at the essential gene in the genome at between 4 days and 9 days (e.g., at 4 days, 5 days, 6 days, 7 days, 8 days or 9 days) after the population of cells is contacted with the donor template and the RNA-guided nuclease and the guide RNA (e.g., configured together as an RNP) that cause a break within the endogenous coding sequence of the essential gene; and at least 60% of the cells (e.g., at least 60%, at least 65%, at least 70%, at least 75%, at least 80%, at least 85%, at least 90%, at least 95%, at least 98%, or at least 99%) in the population of cells comprise a genomic edit (e.g., a genomic edit resulting in a break and/or a genomic edit resulting in a loss of function) within an endogenous CISH coding sequence, e.g., at between 4 days and 9 days (e.g., at 4 days, 5 days, 6 days, 7 days, 8 days or 9 days) after the population of cells is contacted with an RNA-guided nuclease and a guide RNA (e.g., configured together as an RNP) that cause a genomic edit within the endogenous CISH coding sequence; and at least 60% of the cells (e.g., at least 60%, at least 65%, at least 70%, at least 75%, at least 80%, at least 85%, at least 90%, at least 95%, at least 98%, or at least 99%) in the population of cells comprise a genomic edit (e.g., a genomic edit resulting in a break and/or a genomic edit resulting in a loss of function) within an endogenous TGFβRII coding sequence, e.g., at between 4 days and 9 days (e.g., at 4 days, 5 days, 6 days, 7 days, 8 days or 9 days) after the population of cells is contacted with an RNA-guided nuclease and a guide RNA (e.g., configured together as an RNP) that cause a genomic edit within the endogenous TGFβRII coding sequence.

In some embodiments, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells expresses FcγRIII (CD16) or variant thereof and mbIL-15, e.g., at between 4 days and 9 days (e.g., at 4 days, 5 days, 6 days, 7 days, 8 days or 9 days) after the population of cells is contacted with the RNA-guided nuclease and the guide RNA (e.g., configured together as an RNP) and the donor template. In some embodiments, at least 50% (e.g., at least 50%, at least 55%, at least 60%, at least 65%, at least 70%, at least 75%, at least 80%, at least 85%, at least 90%, at least 95%, at least 98%, or at least 99%) of the cells in the population of cells expresses FcγRIII (CD16) or variant thereof and mbIL-15, e.g., at between 4 days and 9 days (e.g., at 4 days, 5 days, 6 days, 7 days, 8 days or 9 days) after the population of cells is contacted with the RNA-guided nuclease and the guide RNA (e.g., configured together as an RNP) and the donor template, and at least 60% of the cells (e.g., at least 60%, at least 65%, at least 70%, at least 75%, at least 80%, at least 85%, at least 90%, at least 95%, at least 98%, or at least 99%) in the population of cells do not express CISH or TGFβRII after the population of cells is contacted with an RNA-guided nuclease and guide RNAs (e.g., configured as an RNP) that cause a genomic edit within the endogenous CISH and TGFβRII coding sequences.

### BRIEF DESCRIPTION OF THE DRAWING

The teachings described herein will be more fully understood from the following description of various exemplary embodiments, when read together with the accompanying drawing. It should be understood that the drawing described below is for illustration purposes only and is not intended to limit the scope of the present teachings in any way.

FIG. 1 shows the locations on the GAPDH gene where exemplary AsCpf1 (AsCas12a) guide RNAs bind, and the results of screening the exemplary guide RNAs that target the GAPDH gene three days after transfection. Results are from gDNA from living cells.

FIG. 2 shows results of screening the exemplary AsCpf1 (AsCas12a) guide RNAs that target the GAPDH gene, three days after transfection. Results are from gDNA from living cells.

FIG. 3A shows an exemplary integration strategy that targets an essential gene according to certain embodiments of the present disclosure. In particular embodiments, introducing a double strand break using CRISPR gene editing (e.g., by Cas12a, Cas9, Cas12b, Cas12c, Cas12e, CasX, or CasΦ (Cas12j), or a variant thereof, e.g., a variant with a high editing efficiency, e.g., capable of editing about 60% to 100% of cells in a population of cells) within a terminal exon (e.g., within about 500 bp upstream (5′) of the stop codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 3B shows an exemplary integration strategy that targets the GAPDH gene according to certain embodiments of the present disclosure. Although FIG. 3B shows a strategy wherein the GAPDH gene is modified in an induced pluripotent stem cell (iPSC), this strategy can be applied to a variety of cell types, including primary cells, e.g., T cells, NK cells, stem cells, iPSCs, and cells differentiated from iPSCs, e.g., iPSC-derived T cells or NK cells for treating cancer.

FIG. 3C shows an exemplary integration strategy that targets the GAPDH gene according to certain embodiments of the present disclosure. The diagram shows that the only cells that should survive over time are those cells that underwent targeted integration of a cassette that restores the GAPDH locus and includes a cargo of interest, as well as unedited cells. The population of unedited cells following CRISPR editing should be small if the nuclease and guide RNA are highly effective at cleaving the essential gene target site and introduce indels that significantly reduce the function of the essential gene product.

FIG. 3D shows an exemplary integration strategy that targets an essential gene according to certain embodiments of the present disclosure. In particular embodiments, introducing a double strand break using CRISPR gene editing (e.g., by Cas12a, Cas9, Cas12b, Cas12c, Cas12e, CasX, or CasΦ (Cas12j), or a variant thereof, e.g., a variant with a high editing efficiency, e.g., capable of editing about 60% to 100% of cells in a population of cells) to target a 5′ exon (e.g., within about 500 bp downstream (3′) of a start codon of the essential gene) and administering a donor plasmid with homology arms designed to mediate homology directed repair (HDR) at the cleavage site, results in a population of viable cells carrying a cargo of interest integrated at the essential gene locus. Those cells that were edited by the CRISPR nuclease, but failed to undergo integration of the cargo at the essential gene locus, do not survive.

FIG. 4 shows editing efficiency at different concentrations (0.625 μM to 4 μM) of an exemplary AsCpf1 (AsCas12a) guide RNA that targets the GAPDH gene.

FIG. 5 shows the knock-in (KI) efficiency of a CD47 encoding “cargo” in the GAPDH gene 4 days post-electroporation when the dsDNA plasmid (“PLA”) was also present. Knock-in efficiency was measured with two different concentrations of the plasmid. Knock-in was measured using ddPCR targeting the 3′ positions of the knock-in “cargo”.

FIG. 6 shows the knock-in efficiency of a CD47 encoding “cargo” in the GAPDH gene 9 days post-electroporation when the dsDNA plasmid was also present. Knock-in was measured using ddPCR both targeting the 5′ and 3′ positions of the knock-in “cargo”, increasing the reliability of the result.

FIG. 7 shows the efficiency of integration of a knock-in cassette, comprising a GFP protein encoding “cargo” sequence, into the GAPDH locus of iPSCs, measured 7 days following transfection. (A) Depicts exemplary microscopy (brightfield and fluorescent) images, and (B) depicts exemplary flow cytometry data. Images and flow cytometry data depict insertion rates for cargo transfection alone (PLA1593 or PLA1651) compared to cargo and guide RNA transfections (RSQ22337+PLA1593 or RSQ24570+PLA1651), additionally, insertion rates with an exemplary exonic coding region targeting guide RNA with appropriate cargo (RSQ22337+PLA1593) are compared to insertion rates with an intronic targeting guide RNA with appropriate cargo (RSQ24570+PLA1651).

FIG. 8A depicts a schematic representation of a bicistronic knock-in cassette (e.g., comprising two cistrons separated by a linker) for insertion into the GAPDH locus. The leading GAPDH Exon 9 coding region and exogenous sequences encoding proteins of interest are separated by linker sequences, and the second GAPDH allele can comprise a target knock-in cassette insertion, indels, or is wild type (WT).

FIG. 8B depicts a schematic representation of bi-allelic knock-in cassettes for insertion into the GAPDH locus. Exogenous “cargo” sequences encoding proteins of interest are located on different knock-in cassettes. For each construct, the leading GAPDH Exon 9 coding region is separated from an exogenous sequence encoding a protein of interest by a linker sequence.

FIG. 9A depicts a schematic representation of a bicistronic knock-in cassette for insertion into the GAPDH locus, with the leading GAPDH Exon 9 coding region and exogenous sequences encoding GFP and mCherry separated by linker sequences P2A, T2A, and/or IRES.

FIG. 9B is a panel of exemplary microscopic images (brightfield and fluorescent) of iPSCs nine days following nucleofection of RNPs comprising RSQ22337 (SEQ ID NO: 95) targeting GAPDH and Cas12a (SEQ ID NO: 62) and a bicistronic knock-in cassette comprising “cargo” sequence encoding GFP and mCherry molecules inserted at the GAPDH locus. iPSCs comprising exemplary “cargo” molecules PLA1582 (comprising donor template SEQ ID NO: 41) with linkers P2A and T2A, PLA1583 (comprising donor template SEQ ID NO: 42) with linkers T2A and P2A, and PLA1584 (comprising donor template SEQ ID NO: 43) with linkers T2A and IRES are shown. Results show that at least two different cargos can be inserted in a bicistronic manner and expression is detectable irrespective of linker type used. All images were taken at 2×100 μm on a Keyence Microscope.

FIG. 9C depicts expression quantification (Y axis) of exemplary “cargo” molecules GFP and mCherry from various bicistronic molecules comprising the described linker pairs (X axis). mCherry as a sole “cargo” protein was utilized as a relative control.

FIG. 10A depicts exemplary flow cytometry data for bi-allelic GFP and mCherry knock-in at the GAPDH gene.

FIG. 10B depicts fluorescence imaging of cell populations prior to flow cytometry analysis following bi-allelic GFP and mCherry knock-in at the GAPDH gene.

FIG. 10C are histograms depicting exemplary flow cytometry analysis data for bi-allelic GFP and mCherry knock-in at the GAPDH gene. Cells were nucleofected with 0.5 μM RNPs comprising Cas12a (SEQ ID NO: 62) and RSQ22337 (SEQ ID NO: 95), and 2.5 μg (5 trials) or 5 μg (1 trial) GFP and mCherry donor templates.

FIG. 11A depicts exemplary flow cytometry data for GFP expression in iPSCs seven days after being transfected with a gRNA and an appropriate donor template comprising a knock-in cassette with a “cargo” sequence encoding GFP that was recombined into various loci.

FIG. 11B depicts the percentage of cells having editing events as measured by Inference of CRISPR Edits (ICE) assays 48 hours after being transfected with the noted gRNA.

FIG. 11C depicts relative integrated “cargo” (GFP) expression intensity as determined by flow cytometry conducted with a FITC channel to filter GFP signal for iPSCs transfected with the noted exemplary gRNA and knock-in cassette combinations.

FIG. 11D depicts relative integrated “cargo” (GFP) expression intensity as determined by flow cytometry conducted with a FITC channel to filter GFP signal for iPSCs transfected with exemplary gRNA targeting the noted essential gene. Knock-in efficiency at each essential gene is denoted by a percentage.

FIG. 12 depicts exemplary flow cytometry data highlighting the efficiency of integration of a donor template comprising a knock-in cassette comprising a GFP protein encoding “cargo” sequence into the TBP locus of iPSCs.

FIG. 13 is exemplary ddPCR results describing knock-in cassette integration ratios in GAPDH or TBP alleles in an iPSC population.

FIG. 14 is a histogram representation of exemplary flow cytometry data for AAV6 mediated knock-in of GFP into T cells using RNPs comprising RSQ22337 targeting GAPDH and Cas12a (SEQ ID NO: 62) at various concentrations of RNP and various AAV6 multiplicity of infection (MOI) rates (vg/cell) measured seven days after electroporation and transduction. The Y axis represents percentage of the cell population expressing GFP, while the X axis depicts AAV6 MOI.

FIG. 15 is a histogram representation of exemplary flow cytometry data depicting cell viability following AAV6 mediated knock-in of GFP at the GAPDH gene in differentiated cells. Depicted is T cell viability four days after AAV6 mediated transduction of a GFP cargo and electroporation with 1 μM RNPs comprising RSQ22337 and Cas12a (SEQ ID NO: 62); the Y axis notes cell viability as a function of total cell population, while the X axis lists various MOIs used to transduce the cells.

FIG. 16A depicts exemplary flow cytometry charts for a population of T cells transduced by AAV6 comprising a knock-in GFP cargo targeting GAPDH at 5E4 MOI and transformed with 4 μM RNP comprising Cas12a (SEQ NO: 62) and RSQ22337.

FIG. 16B depicts exemplary control experiment flow cytometry charts for T cells that were not transduced by AAV6, but solely transformed with 4 μM RNP comprising Cas12a (SEQ NO: 62) and RSQ22337.

FIG. 17A are histograms depicting exemplary flow cytometry data for AAV6 mediated knock-in of GFP into T cells at either the GAPDH locus using RNPs comprising RSQ22337 and Cas12a (SEQ ID NO: 62), or at the TRAC locus. Integration constructs each comprised homology arms approximately 500 bp in length, and T cells were transduced with the same concentration of RNP and AAV MOI. The mean and standard deviation of three independent biological replicates is shown, significant differences in targeted integration were observed (p=0.0022 using unpaired t-test).

FIG. 17B depicts an exemplary flow cytometry chart for a population of T cells transduced by AAV6 comprising a GFP cargo targeted for knock-in at GAPDH at 5E4 MOI and transformed with 4 μM of RNPs comprising Cas12a (SEQ NO: 62) and RSQ22337.

FIG. 17C depicts exemplary expansion and viability data for a population of T cells transduced by AAV6 and transformed with RNPs as described in FIG. 17B, and for a population of T cells that did not undergo RNP transfection (“mock”).

FIG. 17D depicts exemplary flow cytometry data for AAV6 mediated knock-in of GFP into T cells at either the GAPDH locus using RNPs comprising RSQ22337 and Cas12a (SEQ ID NO: 62), as described in FIG. 17B, or at the TRAC locus. Integration constructs each comprised homology arms approximately 500 bp in length, and T cells were transduced with the same concentration of RNPs and AAV MOI. Three independent biological replicates are shown, significant differences in targeted integration were observed (p=<0.001 using unpaired t-test).

FIG. 17E depicts exemplary flow cytometry data for AAV6 mediated knock-in of GFP into T cells at either the GAPDH locus (GAPDH KI) using RNPs comprising RSQ22337 and Cas12a (SEQ ID NO: 62), or at the TRAC locus (TRAC KI). Knock-in efficiency was examined at varying concentrations of AAV6. Integration constructs each comprised homology arms approximately 500 bp in length. The X-axis quantifies AAV6 concentration (vg/ml), while the Y-axis quantifies the percentage of cells that are expressing GFP as detected by flow cytometry. Three independent biological replicates are shown per each knock-in location at each AAV6 concentration. Significant differences in EC50 for AAV6 concentration were observed. ****p=<0.0001 (unpaired t-test).

FIG. 18A is a histogram depicting the knock-in efficiency of CD16 encoding “cargo” integrated at the GAPDH gene of iPSCs. Targeting integration (TI) was measured at day 0 and day 19 of bulk edited cell populations using ddPCR targeting the 5′ (5′ assay) and 3′ (3′ assay) positions of the knock-in cargo.

FIG. 18B is a histogram depicting the genotypes of iPSC clones with CD16 encoding “cargo” integrated at the GAPDH gene, measured using ddPCR targeting the 5′ (5′ CDN probe) and 3′ (3′ Poly A probe) positions of the knock-in cargo. Shown are results for four exemplary cell lines, two lines were classified as homozygous knock-in with targeted integration (TI) rates of 88.5% (clone 1) and 90.5% (clone 2) respectively, and two lines were classified as heterozygous knock-in with TI rates of 45.6% (clone 1) and 46.5% (clone 2) respectively.

FIG. 19A depicts exemplary flow cytometry data from day 32 of homozygous clone 1 CD16 knock-in iPSCs differentiated into iNKs. The data highlights the efficiency of integration and high expression (e.g., approximately 98%) of a knock-in cassette comprising a CD16 protein encoding “cargo” sequence into the GAPDH gene of iPSCs. In addition, the data shows knock-in of a “cargo” at the GADPH gene does not inhibit the differentiation process, as represented by high CD56+CD45+ population proportions.

FIG. 19B depicts exemplary flow cytometry data from day 32 of homozygous clone 2 CD16 knock-in iPSCs differentiated into iNKs. The data highlights the efficiency of integration and expression of a knock-in cassette comprising a CD16 protein encoding “cargo” sequence into the GAPDH gene of iPSCs.

FIG. 19C depicts exemplary flow cytometry data from day 32 of heterozygous clone 1 CD16 knock-in iPSCs differentiated into iNKs. The data highlights the efficiency of integration and high expression (e.g., approximately 97.8%) of a knock-in cassette comprising a CD16 protein encoding “cargo” sequence into the GAPDH gene of iPSCs.

FIG. 19D depicts exemplary flow cytometry data from day 32 of heterozygous clone 2 CD16 knock-in iPSCs differentiated into iNKs. The data highlights the efficiency of integration and expression of a knock-in cassette comprising a CD16 protein encoding “cargo” sequence into the GAPDH gene of iPSCs.

FIG. 20 is a schematic representation of an exemplary solid tumor cell killing assay, depicting the use of knock-in iPSCs differentiated into iNK cells to kill 3D spheroids created from a cancer cell line (e.g., SK-OV-3 ovarian cancer cells). Antibodies and/or cytokines may optionally be added during the 3D spheroid killing stage.

FIG. 21A shows the results of a solid tumor killing assay as described in FIG. 20. Homozygous clones comprising CD16 knock-in at the GAPDH gene were differentiated into iNK cells and functioned to reduce tumor cell spheroid size, particularly following the addition of an antibody, e.g., 10 μg/mL trastuzumab; addition of an antibody promotes antibody dependent cellular cytotoxicity (ADCC) and tumor cell killing by iNKs. Control “WT PCS” cells were bulk unedited parental clones that were electroporated without RNPs or plasmids, and at the same stage of iNK cell differentiation as test cells. The Y axis depicts normalized total integrated red object intensity, a proxy for tumor cell abundance, while the X axis depicts the Effector to Target cell (E:T) ratio.

FIG. 21B shows the results of a solid tumor killing assay as described in FIG. 20. Heterozygous clones comprising CD16 knock-in at the GAPDH gene were differentiated into iNK cells and functioned to reduce tumor cell spheroid size, particularly following the addition of an antibody, e.g., 10 μg/mL trastuzumab; addition of an antibody promotes ADCC and tumor cell killing by iNKs. Control “WT PCS” cells were bulk unedited parental clones that were electroporated without RNPs or plasmids, and at the same stage of iNK cell differentiation as test cells. The Y axis depicts normalized total integrated red object intensity, a proxy for tumor cell abundance, while the X axis depicts the E:T ratio.

FIG. 22 shows the results of an in vitro serial killing assay, where homozygous or heterozygous clones comprising CD16 knock-in at the GAPDH gene were differentiated into iNK cells and were serially challenged with hematological cancer cells (e.g., Raji cells), with or without the addition of antibody (0.1 μg/mL rituximab). The X axis represents time (0-598 hr.) with an additional tumor cell bolus (5,000 cells) being added approximately every 48 hours, and the Y axis represents killing efficacy as measured by normalized total red object area (e.g., presence of tumor cells). Star (*) denotes onset of addition of 0.1 μg/mL rituximab in previously rituximab absent trials. The data shows that edited iNK cells (CD16 knock-in at GAPDH gene; clones “Homo_C1”, “Homo_C2”, “Het_C1”, and “Het_C2”) continue to kill hematological cancer cells while unedited (“PCS”) or control edited iNKs (“GFP Bulk”) derived from parental iPSCs lose this function at equivalent time points.

FIG. 23 depicts a correlation (R² of 0.768) between CD16 expression and reduction in tumor spheroid size at an Effector to Target (E:T) ratio of 3.16:1. Shown are differentiated iNK cells derived from either iPSC bulk edited cells or iPSC individual clones with CD16 knock-in at the GAPDH gene. The Y axis represents normalized tumor cell killing values, while the X axis represents the percentage of a cell population expressing CD16.

FIG. 24A is a histogram depicting exemplary ddPCR data measured at day 9 post nucleofection of two different iPSC lines with plasmids and 2 μM RNPs comprising RSQ22337 targeting the GAPDH gene and Cas12a (SEQ ID NO: 62), for knock-in of CD16 cargo, a CAR cargo, or a biallelic GFP/mCherry cargo into the GAPDH gene.

FIG. 24B depicts exemplary flow cytometry data from iPSC lines edited with plasmids and 2 μM RNPs comprising RSQ22337 targeting the GAPDH gene and Cas12a (SEQ ID NO: 62) for knock-in of CXCR2 cargo into the GAPDH gene (GAPDH::CXCR2), or control iPSCs transformed with RNP only (Wild-type). CXCR2 expression is noted on the X axis, edited cells expressing CXCR2 were 29.2% of the bulk edited cell population, while surface expression of CXCR2 was 8.53% of the bulk edited cell population.

FIG. 25 is a histogram depicting the knock-in efficiency of a series of knock-in cassette cargo sequences such as CD16-P2A-CAR, CD16-IRES-CAR, CAR-P2A-CD16, CAR-IRES-CD16, and mbIL-15 into the GAPDH gene using RNPs comprising RSQ22337 targeting the GAPDH gene and Cas12a (SEQ ID NO: 62), measured on day 0 post-electroporation using ddPCR targeting the 5′ (5′ CDN probe) and 3′ (3′ Poly A probe) positions of the knock-in “cargo”.

FIG. 26 diagrammatically depicts a membrane-bound IL15.IL15Rα (mbIL-15) construct that can be utilized as a knock-in cargo sequence as described herein.

FIG. 27 is a histogram depicting the TI of mbIL-15 into the GAPDH gene when measured as a percentage of a bulk edited population. Shown are TI rates from iPSCs that that are on day 28 of the differentiation to iNK cell process.

FIG. 28A depicts exemplary flow cytometry data from bulk edited mbIL-15 GAPDH gene knock-in iPSC populations at day 39 of differentiation into iNKs.

FIG. 28B depicts exemplary flow cytometry data from bulk edited mbIL-15 GAPDH gene knock-in iPSC populations at day 39 of differentiation into iNKs.

FIG. 28C shows surface expression phenotypes (measured as a percentage of the population) of bulk edited mbIL-15 GAPDH gene knock-in iPSC populations being differentiated into iNK cells as compared to parental clone cells also being differentiated into iNK cells (“WT”) at day 32, day 39, day 42, and day 49 of iPSC differentiation.

FIG. 29 shows the results from two in-vitro tumor cell killing assays. Two biological replicates of bulk edited iPSC populations (S1 and S2) comprising mbIL-15 knock-in at the GAPDH gene were differentiated into iNK cells (day 56 of differentiation for S2, and day 63 of differentiation for S1) and functioned to reduce hematological cancer cells (e.g., Raji cells) fluorescence signal when compared to WT parental cells also differentiated into iNK cells, measured in the absence or presence of 10 μg/mL rituximab, E:T ratios of 1 (A) or 2.5 (B); (experiments performed in duplicate, R1 and R2).

FIG. 30A shows the results of a solid tumor killing assay as described in FIG. 20. Two biological replicates of bulk edited iPSC populations (S1 and S2) comprising mbIL-15 knock-in at the GAPDH gene were differentiated into iNK cells (day 39 of iPSC differentiation) and functioned to reduce tumor cell spheroid size when compared to WT parental cells also differentiated into iNK cells. Addition of 5 ng/mL exogenous IL-15 increased tumor cell killing by iNKs. The Y axis depicts normalized total integrated red object intensity, a proxy for tumor cell abundance, while the X axis depicts E:T ratio.

FIG. 30B shows the results of solid tumor killing assays as described in FIG. 20. Two biological replicates of bulk edited iPSC populations (S1 and S2) comprising mbIL-15 knock-in at the GAPDH gene were differentiated into iNK cells (day 39 of differentiation) and functioned to reduce tumor cell spheroid size when compared to WT parental cells also differentiated into iNK cells at corresponding stages of differentiation and E:T ratios (shown is an E:T ratio of approximately 31.6). Addition of 5 ng/mL exogenous IL-15 was necessary for robust WT iNK cell spheroid reduction, while mbIL-15 KI iNK cells were able to reduce tumor volume without exogenous IL-15. X axis represents time (0-100 hr) while the Y axis represents killing efficacy as measured by normalized total red object area (e.g., presence of tumor cells).

FIG. 30C shows the results of solid tumor killing assays as described in FIG. 20. Two biological replicates of bulk edited populations (S1 and S2) comprising mbIL-15 knock-in at the GAPDH gene were differentiated into iNK cells (e.g., at day 39, day 42, day 49, day 56, and day 63 of differentiation) and functioned to reduce tumor cell spheroid size when compared to WT parental cells at corresponding stages of iNK cell differentiation (experiments performed in duplicate, R1 and R2).

FIG. 30D shows the results of solid tumor killing assays as described in FIG. 20. Two biological replicates of bulk edited iPSC populations (S1 and S2) comprising mbIL-15 knock-in at the GAPDH gene were differentiated into iNK cells (e.g., at day 39, day 42, day 49, day 56, and day 63 of differentiation; in duplicate R1 and R2) and functioned to reduce tumor cell spheroid size when compared to WT parental cells at corresponding stages of iNK cell differentiation (experiments performed in duplicate, R1 and R2). Cell populations were supplemented with exogenous IL-15 (5 ng/mL), leading to more robust iNK cell induced spheroid reduction at each stage of maturation tested when compared to non-supplemented cells (FIG. 30C) (experiments performed in duplicate, R1 and R2).

FIG. 31A shows the results of solid tumor killing assays as described in FIG. 20. Two biological replicates of bulk edited iPSC populations (S1 and S2) comprising mbIL-15 knock-in at the GAPDH gene were differentiated into iNK cells (day 63 of iPSC differentiation for S1, and day 56 of iPSC differentiation for S2) and functioned to reduce tumor cell spheroid size. The Y axis represents killing efficacy as measured by normalized total red object area (e.g., presence of tumor cells), while the X axis represents the E:T cell ratio; experiments were performed in duplicate or triplicate, R1, R2, and R2.1.

FIG. 31B shows the results of solid tumor killing assays as described in 31A, but with the addition of 10 μg/mL Herceptin antibody, an addition that triggers ADCC tumor cell killing.

FIG. 31C shows the results of solid tumor killing assays as described in 31A, but with the addition of 5 ng/ml exogenous IL-15.

FIG. 31D shows the results of solid tumor killing assays as described in 31A, but with the addition of 5 ng/ml exogenous IL-15 and 10 μg/mL Herceptin antibody, an addition that triggers ADCC tumor cell killing.

FIG. 32 depicts the cumulative results of two independent sets of cells and 3-5 repeats of solid tumor killing assays as described in FIG. 20. Two independent bulk edited populations (S1 and S2) comprising mbIL-15 knock-in at the GAPDH gene were differentiated into iNK cells (day 39 and 49 of iPSC differentiation for set 1, and day 42 of iPSC differentiation for S2) and functioned to significantly reduce tumor cell spheroid size when compared to differentiated WT parental cell iNKs in the absence of exogenous IL-15 (P=0.034, +/−standard deviation, unpaired t-test); in addition, differentiated knock-in cells trended towards significant reduction of tumor cell spheroid size when compared to differentiated WT parental cells in the presence of 5 ng/mL exogenous IL-15 (P=0.052, +/−standard deviation, unpaired t-test).

FIG. 33A schematically depicts a knock-in cassette cargo sequence comprising membrane-bound IL15.IL15Rα (mbIL-15) coupled with a GFP sequence, for integration at a target gene as described herein.

FIG. 33B schematically depicts a knock-in cassette cargo sequence comprising CD16, IL15, and IL15Rα, for integration at a target gene as described herein.

FIG. 33C schematically depicts a knock-in cassette cargo sequence comprising CD16 and membrane bound IL15.IL15Rα (mbIL-15), for integration at a target gene as described herein.

FIG. 34A depicts exemplary flow cytometry data from bulk edited iPSC populations seven days after transformation with PLA1829 (see FIG. 33A) comprising a cargo sequence of membrane-bound IL15.IL15Rα (mbIL-15) coupled with a GFP sequence inserted in the GAPDH gene using RNPs comprising RSQ22337 targeting the GAPDH gene and Cas12a (SEQ ID NO: 62), or control WT cells transformed with RNPs only, measured using ddPCR. Shown on the Y axis is IL-15Rα expression, while GFP expression is shown on the X axis.

FIG. 34B depicts exemplary flow cytometry data from bulk edited iPSC populations seven days after transformation with PLA1832 or PLA1834 (see FIGS. 33B and 33C), comprising a cargo sequence of CD16, IL-15, and IL 15Rα, or comprising a cargo sequence of CD16 and membrane-bound IL15.IL15Rα (mbIL-15); inserted in the GAPDH gene using RNPs comprising RSQ22337 targeting the GAPDH gene and Cas12a (SEQ ID NO: 62), measured using ddPCR. Shown on the Y axis is IL-15Rα expression, X axis is GFP expression.

FIG. 35A is a histogram depicting the genotypes of individual colonies following transformation as described in FIG. 34A with PLA1829 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 35B is a histogram depicting the genotypes of individual colonies following transformation as described in FIG. 34B with PLA 1832 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 35C is a histogram depicting the genotypes of individual colonies following transformation as described in FIG. 34B with PLA1834 (5 μg) and 2 μM RNPs comprising RSQ22337 targeting the GAPDH gene and Cas12a (SEQ ID NO: 62), measured using ddPCR. Shown are individual homozygous (˜100% TI), heterozygous (˜50% TI), or wild type (˜0% TI) cells.

FIG. 36A depicts exemplary flow cytometry data from cells comprising knock-in cargo sequences from PLA1829, PLA1832, or PLA1834 at the GAPDH gene (as described in FIG. 34A-34C) measured at day 32 of differentiation into iNKs; “WT” cells were transformed with RNPs only and were also at day 32 of differentiation into iNKs. The data highlights the efficiency of integration and expression of knock-in cassettes comprising an IL-15Rα protein encoding “cargo” sequence. The Y axis quantifies the percentage of cells from the noted population that are expressing IL-15Rα, while the X axis denotes colony genotype.

FIG. 36B depicts exemplary flow cytometry data from cells comprising knock-in cargo sequences from PLA1829, PLA1832, or PLA1834 at the GAPDH gene (as described in FIG. 34A-34C) measured at day 32 of differentiation into iNKs; “WT” cells were transformed with RNPs only and were also at day 32 of differentiation into iNKs. The data highlights the efficiency of integration and expression of knock-in cassettes comprising a CD16 protein encoding “cargo” sequence. The Y axis quantifies the percentage of cells from the noted population that are expressing CD16, while the X axis denotes colony genotype.

FIG. 36C depicts exemplary flow cytometry data from cells comprising knock-in cargo sequences from PLA1829, PLA1832, or PLA1834 at the GAPDH gene (as described in FIG. 34A-34C) measured at day 32 of differentiation into iNKs; “WT” cells were transformed with RNPs only and were also at day 32 of differentiation into iNKs. The data highlights the efficiency of integration and expression of knock-in cassettes comprising an IL-15Rα protein encoding “cargo” sequence. The Y axis quantifies the median fluorescence intensity (MFI) of a cell population expressing IL-15Rα, while the X axis denotes colony genotype.

FIG. 36D depicts exemplary flow cytometry data from cells comprising knock-in cargo sequences from PLA1829, PLA1832, or PLA1834 at the GAPDH gene (as described in FIG. 34A-34C) measured at day 32 of differentiation into iNKs; “WT” cells were transformed with RNPs only and were also at day 32 of differentiation into iNKs. The data highlights the efficiency of integration and expression of knock-in cassettes comprising a CD16 protein encoding “cargo” sequence. The Y axis quantifies the median fluorescence intensity (MFI) of a cell population expressing CD16, while the X axis denotes colony genotype.

FIG. 36E shows exemplary flow cytometry data from unedited (WT) cells or homozygous cells comprising knock-in cargo sequences from PLA1834 at the GAPDH locus (CD16^{+/+}/mbIL-15^{-/-}). The data highlights the efficiency of integration and expression of knock-in cassettes comprising a CD16 and IL-15Rα protein encoding cargo sequence. The Y axis quantifies the percentage of cells from the noted population that are expressing the selected gene, while the X axis denotes whether the selected gene is CD16 or IL-15Rα.

FIG. 36F depicts exemplary flow cytometry data from iNK cells comprising knock-in cargo sequences from PLA1829 or PLA1834 at the GAPDH gene, or from WT cells, before or after cytotoxicity assay in the absence of trastuzumab (Herceptin).

FIG. 36G depicts exemplary flow cytometry data from iNK cells comprising knock-in cargo sequences from PLA1829 or PLA1834 at the GAPDH gene, or from WT cells, before or after cytotoxicity assay in the presence of trastuzumab (Herceptin).

FIG. 36H depicts CD16 surface expression from two independent flow cytometry analyses of homozygous iNK cells comprising knock-in cargo sequences from PLA1834 at the GAPDH gene (CD16^{+/+}/mbIL-15^{+/+}), or unedited (WT) cells. CD16 surface expression was assessed before or after a 2D cell killing (LDH) assay and in absence or presence of trastuzumab. The Y axis quantifies the percentage of cells from the noted population that are CD56/CD16+, while the X axis denotes whether the sample was before or after the 2D killing assay.

FIG. 36I depicts percent cytotoxicity demonstrated by homozygous PLA1834-transformed (CD16^{+/+}/mbIL-15^{+/+}) iNK cells or unedited (WT) iNK cells in a 2D cell killing assay (LDH assay). Assays were performed in the presence or absence of 10 μg/ml trastuzumab at an E:T ratio of 1 (left) or 2.5 (right). The Y axis quantifies the percent cytotoxicity, while the X axis denotes the presence or absence of trastuzumab. *p<0.05, **p<0.01 (two-way ANOVA).

FIG. 36J depicts total cell number (left panel) of iNK cells comprising knock-in cargo sequences from PLA1829 or PLA1834 at the GAPDH gene, or of unedited (WT) iNK cells, following an in vitro persistence assay in the absence of the cytokines, IL-2 and IL-15. Fold change of cells comprising a knock-in from PLA1834 relative to cells comprising a homozygous knock-in from PLA1829 is shown in the top right panel. Fold change of cells comprising a homozygous knock-in from PLA1834 (CD16^{+/+}/mbIL-15^{+/+}) relative to unedited (WT) cells is shown in the bottom right panel.

FIG. 37A shows the results of a solid tumor killing assay as described in FIG. 20. Clones comprising homozygous CD16 knock-in at the GAPDH gene were differentiated into iNK cells and functioned to reduce tumor cell spheroid size, particularly following the addition of an antibody, e.g., 10 μg/mL trastuzumab. The addition of an antibody promotes antibody dependent cellular cytotoxicity (ADCC) and tumor cell killing by iNKs. Control “WT” cells were bulk unedited parental clones that were electroporated without RNPs or plasmids and were at the same stage of iNK cell differentiation as test cells. The Y axis depicts normalized total integrated red object intensity, a proxy for tumor cell abundance, while the X axis depicts the Effector to Target cell (E:T) ratio. The IC50 for “WT” cells was an E:T ratio of 3.0, while the IC50 for SLEEK CD16 KI cells was an E:T ratio of 0.5.

FIG. 37B shows the results of a 3D tumor spheroid killing assay conducted as depicted in FIG. 20. Homozygous PLA1834-transformed (CD16^{+/+}/mbIL-15^{-/-}) iNK cells and unedited (WT) iNK cells were introduced to SK-OV-3 tumor cells at an E:T ratio of 10 in the absence (left panels) or presence (right panels) of 10 μg/ml trastuzumab. The top panels display imaging of the tumor spheroid at 0 hours and 100 hours with visibility of the red object signal used to measure tumor cell abundance. The bottom panels display spheroid size as measured via the integrated red object intensity on the Y axis and time in hours on the X axis.

FIG. 37C shows the results of 3D tumor spheroid killing assays conducted as depicted in FIG. 20. Unedited (WT) iNK cells, peripheral blood NK cells, and two clones of homozygous PLA1834-transformed (CD16^{+/+}/mbIL-15^{+/+}) iNK cells were used against SK-OV-3 tumor cells at varying E:T ratios. In the left panels, 5 ng/ml exogenous IL-15 and 10 μg/ml trastuzumab was present. Two independent experiments were performed for each type of cell or clone with the exception of one experiment for the peripheral blood NK cells. IC50 values based on the top left panel are presented in the table in the bottom left panel and highlight the greater efficacy of the CD16^{+/+}/mbIL-15^{+/+}iNK cells in killing tumor cells. The right panel displays IC50 values from 3D tumor spheroid killing assays for homozygous PLA1834-transformed (CD16^{+/+}/mbIL-15^{+/+}) iNK cells and unedited (WT) iNK cells in the absence and presence of 10 μg/ml trastuzumab. *p<0.05, **p<0.01 (unpaired t-test).

FIG. 38A depicts percent cytotoxicity demonstrated by mbIL-15/CD16 (CD16^{+/+}/mbIL-15^{+/+}) DKI iNK cells or unedited (WT) iNK cells in a lactate dehydrogenase (LDH) cytotoxicity assay. Three different clones (A2, A4, C4) of mbIL-15/CD16 (CD16^{+/+}/mbIL-15^{+/+}) DKI iNK cells were tested. Assays were performed in the presence or absence of 10 μg/ml trastuzumab and at an E:T ratio of 1. The Y axis quantifies the percent cytotoxicity, while the X axis denotes the iNK cells examined. Error bars denote standard deviation.

FIG. 38B depicts flow cytometry data of unedited (WT) and mbIL-15/CD16 (CD16^{+/+}/mbIL-15^{+/+}) DKI iNK cells. Two clones (A2, A4) of mbIL-15/CD16 (CD16^{+/+}/mbIL-15^{+/+}) DKI iNK cells were examined. Cells were pre-gated for living hCD45+ cells and further analyzed for CD16/CD56 expression. Approximately 100% of mbIL-15/CD16 (CD16^{+/+}/mbIL-15+/+) DKI iNK cells displayed high CD16 expression compared to approximately 50% of WT iNK cells.

FIG. 38C is a schematic of an in vivo tumor killing assay. Mice were intraperitoneally inoculated with 0.25×10⁶ SKOV3-luc cells, and following 2-6 days to allow for tumor establishment, mice were randomized into groups. One day later, mice intraperitoneally received 2×10⁶ or 5×10⁶ mbIL-15/CD16 (CD16^{+/+}/mbIL-15^{+/+}) DKI iNK cells in combination with 2.5 mpk trastuzumab. In some treatment groups, mice received an additional dose of 2.5 mpk trastuzumab at 35 days (as indicated by the arrowhead) or at 21, 28, and 35 days (as indicated by the arrows) post-introduction of iNK cells. Mice were followed for up to 90 days post-introduction of iNK cells. The X axis represents time since introduction of NK cells.

FIG. 38D shows averaged results with standard error of the mean of the in vivo tumor killing assay described in FIG. 38C. Groups of mice are represented by each horizontal line. The groups included mice that received mbIL-15/CD16 (CD16^{+/+}/mbIL-15^{+/+}) DKI iNK cells (DKI iNK) with trastuzumab, trastuzumab alone, or an isotype control. Doses of trastuzumab are indicated by arrows and arrowheads for groups receiving a total of 4 doses or 2 doses, respectively. The X axis represents time since introduction of NK cells, while the Y axis represents tumor burden as measured by bioluminescent imaging (BLI) using an in vivo imaging system (IVIS).

FIG. 38E shows the survival of mice subjected to the in vivo tumor killing assay described in FIG. 38C. Groups of mice are represented by each horizontal line. Mice dosed with mbIL-15/CD16 (CD16^{+/+}/mbIL-15^{+/+}) DKI iNK cells in combination with trastuzumab (5M DKI iNK+Tras.×4, 2M DKI iNK+Tras×2) had prolonged survival compared to mice dosed with trastuzumab alone. The X axis represents time since introduction of NK cells, while the Y axis represents percent survival of the mice.

FIG. 38F shows bioluminescent imaging of mice subjected to the in vivo tumor killing assay described in FIG. 38C. The treatment groups of the mice are denoted along the top of the panel, while the time since introduction of NK cells is denoted along the left side of the panel. The right color scale represents the radiance (p/sec/cm²/sr) of the bioluminescence (from a minimum of 2.23×10⁶ to a maximum of 5.57×10⁷) as seen in the images.

FIG. 38G shows flow cytometry data of cells obtained by peritoneal lavage of mice subjected to the in vivo tumor killing assay described in FIG. 38C. The top row shows data following sacrifice at day 90, from the mouse that received 5×10⁶ mbIL-15/CD16 (CD16^{+/+}/mbIL-15^{+/+}) DKI iNK cells+trastuzumab according to the in vivo tumor killing assay as described in FIG. 38C. The bottom row shows data following sacrifice at day 118, from the mouse that received 2×10⁶ mbIL-15/CD16 (CD16^{+/+}/mbIL-15^{+/+}) DKI iNK cells+trastuzumab according to the in vivo tumor killing assay as described in FIG. 38C. iNK cells (inset boxes in top left and bottom left) were identified by flow cytometry using the human CD46 (hCD46) marker and further analyzed for expression of CD16/CD56. The data highlights that the mbIL-15/CD16 (CD16^{+/+}/mbIL-15^{+/+}) DKI iNK cells persist in vivo for at least 118 days.

FIG. 39A is a schematic of an in vivo tumor killing assay. Mice were intraperitoneally inoculated with 0.25×10⁶ SKOV3-luc cells, and following 2-6 days to allow for tumor establishment, mice were randomized into groups. One day later, mice intraperitoneally received 5×10⁶ (5M) unedited (WT) or mbIL-15/CD16 (CD16^{+/+}/mbIL-15^{+/+}) DKI iNK cells. In some treatment groups, mice received a single dose of 2.5 mpk trastuzumab at introduction of the iNK cells (day 0) or multiple doses of 2.5 mpk trastuzumab at 0, 7, and 14 days (as indicated by the arrows) post-introduction of iNK cells.

FIG. 39B shows tumor burden (median with interquartile range) for the in vivo tumor killing assay described in FIG. 39A. Groups of mice are represented by each horizontal line. Each treatment group had 8 mice. The groups included mice that received unedited (WT) iNK cells, mbIL-15/CD16 (CD16^{+/+}/mbIL-15^{+/+}) DKI iNK cells (DKI iNK), or an isotype control. The mbIL-15/CD16 (CD16^{+/+}/mbIL-15^{+/+}) DKI iNK clone (A2) used corresponds to the A2 clone as identified in FIGS. 35C, 38A, and 38B. The X axis represents time since introduction of NK cells, while the Y axis represents tumor burden as measured by bioluminescent imaging (BLI) using an in vivo imaging system (IVIS).

FIG. 39C shows tumor burden (median with interquartile range) for the in vivo tumor killing assay described in FIG. 39A. Groups of mice are represented by each horizontal line. Each treatment group had 8 mice. The groups included mice that received unedited (WT) iNK cells+trastuzumab, mbIL-15/CD16 (CD16^{+/+}/mbIL-15^{+/+}) DKI iNK cells (DKI iNK)+trastuzumab, trastuzumab alone, or an isotype control. The mbIL-15/CD16 (CD16^{+/+}/mbIL-15^{+/+}) DKI iNK clones (A2, A4) used correspond to the A2 and A4 clones as identified in FIGS. 35C, 38A, and 38B. Dosing of trastuzumab on day 0 is indicated by the arrow. The X axis represents time since introduction of NK cells, while the Y axis represents tumor burden as measured by bioluminescent imaging (BLI) using an in vivo imaging system (IVIS).

FIG. 39D shows the survival of mice subjected to the in vivo tumor killing assay described in FIG. 39A. Groups of mice are represented by each horizontal line. Mice dosed with mbIL-15/CD16 (CD16^{+/+}/mbIL-15^{+/+}) DKI iNK cells in combination with trastuzumab (DKI iNK+Tras.×1) had significantly prolonged survival compared to mice dosed with trastuzumab alone (Trastuzumab×1). The X axis represents time since introduction of NK cells, while the Y axis represents percent survival of the mice. ****p<0.0001 (Log-rank Mantel-Cox test).

FIG. 39E shows tumor burden (median with interquartile range) for the in vivo tumor killing assay described in FIG. 39A. Groups of mice are represented by each horizontal line. Each treatment group had 8 mice. The groups included mice that received unedited (WT) iNK cells in combination with trastuzumab (TRA×3), mbIL-15/CD16 (CD16^{+/+}/mbIL-15^{+/+}) DKI iNK cells in combination with trastuzumab (TRA×3), trastuzumab (TRA×3) alone, or an isotype control. The mbIL-15/CD16 (CD16^{+/+}/mbIL-15^{+/+}) DKI iNK clone used corresponds to the A2 clone as identified in, e.g., FIGS. 35C, 38A, and 38B. Mice dosed with the mbIL-15/CD16 (CD16^{+/+}/mbIL-15^{+/+}) DKI iNK cells+trastuzumab had significantly decreased tumor burden as compared to mice dosed with WT iNK cells+trastuzumab. Doses of trastuzumab on day 0, 7, and 14 are indicated by the arrows. The X axis represents time since introduction of NK cells, while the Y axis represents tumor burden as measured by bioluminescent imaging (BLI) using an in vivo imaging system (IVIS). ***p<0.001 (unpaired t-test).

FIG. 39F shows the survival of mice subjected to the in vivo tumor killing assay described in FIG. 39A. Groups of mice are represented by each horizontal line. Mice dosed with mbIL-15/CD16 (CD16^{+/+}/mIL-15^{+/+}) DKI iNK cells in combination with trastuzumab (×3) had significantly prolonged survival compared to mice dosed with WT iNK cells in combination with trastuzumab (×3). Additionally, mice dosed with either mbIL-15/CD16 (CD16^{+/+}/mIL-15^{+/+}) DKI iNK cells+trastuzumab (×3) or WT iNK cells+trastuzumab (×3) had a significantly greater probability of survival as compared to trastuzumab alone (TRA×3, TRA×1). The X axis represents time since introduction of NK cells, while the Y axis represents percent survival of the mice. *p<0.05 (unpaired t-test).

FIG. 39G shows measured tumor burden per mouse on day 33 of the in vivo tumor killing assay described in FIG. 39A. The left panel depicts data for mice receiving a single dose of trastuzumab (on day 0 post-introduction of iNK cells). The right panel depicts data for mice receiving three doses of trastuzumab (on days 0, 7, and 14 post-introduction of iNK cells). The X axis denotes the treatment group, while the Y axis represents tumor burden as measured by bioluminescent imaging (BLI) using an in vivo imaging system (IVIS). **p<0.01, ****p<0.0001, ns denotes not significant (unpaired t-test).

FIG. 39H shows measured tumor burden per mouse on day 11 (left panel) and day 54 (right panel) of the in vivo tumor killing assay described in FIG. 39A. Mice dosed with mbIL-15/CD16 (CD16^{+/+}/mIL-15^{+/+}) DKI iNK cells in combination with trastuzumab (DKI iNK+Tras.×1) had significantly reduced tumor burden at day 11 and at day 54 as compared to mice dosed with unedited iNK cells in combination with trastuzumab or trastuzumab alone. The X axis denotes the treatment group, while the Y axis represents tumor burden as measured by bioluminescent imaging (BLI) using an in vivo imaging system (IVIS). ***p<0.001, ****p<0.0001 (Mann-Whitney test).

FIG. 39I shows representative bioluminescent imaging of mice subjected to the in vivo tumor killing assay described in FIG. 39A. The treatment groups of the mice are denoted along the top of the panel, while the time since introduction of NK cells is denoted along the left side of the panel. Each treatment group had 8 mice. The table below the images displays the number of tumor free mice/total mice in the treatment group (from top of panel) at day 40 post-introduction of NK cells. The bottom color scale represents the radiance (p/sec/cm²/sr) of the bioluminescence (from a minimum of 2.30×10⁵ to a maximum of 3.72×10⁷) as seen in the images.

FIG. 39J depicts flow cytometry data of cells obtained by peritoneal lavage of mice subjected to the in vivo tumor killing assay described in FIG. 39A. The top row shows representative data following sacrifice at day 144 from mice that received WT iNK cells+trastuzumab (×3) according to the in vivo tumor killing assay as described in FIG. 39A. The bottom row shows representative data following sacrifice at day 144 from mice that received mbIL-15/CD16 (CD16^{+/+}/mIL-15^{+/+}) DKI iNK cells+trastuzumab (×3) according to the in vivo tumor killing assay as described in FIG. 39A. iNK cells (inset boxes in top left and bottom left) were identified by flow cytometry using the human CD45 (hCD45) marker and further analyzed for expression of human CD16 (hCD16) and human CD56 (hCD56). The data highlights that the mbIL-15/CD16 (CD16^{+/+}/mIL-15^{+/+}) DKI iNK cells persist in vivo for at least 144 days and almost all of these cells continue to express CD16 on their surface.

FIG. 40 shows microscopy of cell morphology and flow cytometry of pluripotency markers of human induced pluripotent stem cells (hiPSCs) grown in various media in the absence or presence of Activin A (1 ng/ml or 4 ng/ml ActA).

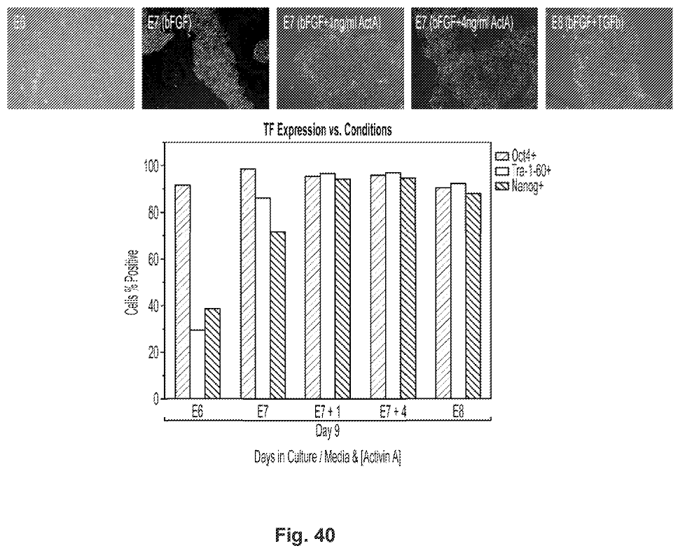

FIG. 41 shows morphology of TGFβRII knockout hiPSCs (clone 7) or CISH/TGFβRII DKO hiPSCs (clone 7) cultured in media with or without Activin A (1 ng/mL, 2 ng/mL, 4 ng/mL, or 10 ng/ml).

FIG. 42 shows morphology of TGFβRII knockout hiPSCs (clone 9) cultured in media with or without Activin A (1 ng/ml, 2 ng/mL, 4 ng/ml, or 10 ng/ml).

FIG. 43A shows the bulk editing rates at the CISH and TGFβRII loci for single knockout and double knockout hiPSCs.

FIG. 43B shows expression of Oct4 and SSEA4 in TGFβRII knockout hiPSCs, CISH knockout hiPSCs, and double knockout hiPSCs cultured in Activin A.

FIG. 44 shows expression of Nanog and Tra-1-60 in TGFβRII knockout hiPSCs, CISH knockout hiPSCs, and double knockout hiPSCs cultured in Activin A.

FIG. 45 is a schematic of the procedure related to the STEMdiff™ Trilineage Differentiation Kit (STEMCELL Technologies Inc.).

FIG. 46A shows expression of differentiation markers of TGFβRII knockout hiPSCs, CISH knockout hiPSCs, and double knockout hiPSCs cultured in Activin A.

FIG. 46B shows karyotypes of TGFβRII/CISH double knockout hiPSCs cultured in Activin A.

FIG. 46C shows an expanded Activin A concentration curve performed on an unedited parental PSC line, an edited TGFβRII KO clone (C7), and an additional representative (unedited) cell line designated RUCDR. The minimum concentration of Activin A required to maintain each line varied slightly with the TGFβRII KO clone requiring a higher baseline amount of Activin A as compared to the parental control (0.5 ng/ml vs 0.1 ng/ml).

FIG. 46D shows the stemness marker expression in an unedited parental PSC line, an edited TGFβRII KO clone (C7), and an unedited RUCDR cell line, when cultured with the base medias alone (no supplemental Activin A). The TGFβRII KO iPSCs did not maintain stemness marker expression while the two unedited lines were able to maintain stemness marker expression in E8.

FIG. 47A is a schematic representation of an exemplary method for creating edited iPSC clones, followed by the differentiation to and characterization of enhanced CD56+ iNK cells.

FIG. 47B is a schematic of an iNK cell differentiation process utilizing STEMDiff APEL2 during the second stage of the differentiation process.

FIG. 47C is a schematic of an iNK cell differentiation process utilizing NK-MACS with 15% serum during the second stage of the differentiation process.

FIG. 47D shows the fold-expansion of unedited PCS-derived iNK cells and the percentage of iNK cells expressing CD45 and CD56 at day 39 of differentiation when differentiated using NK-MACS or Apel2 methods as depicted in FIG. 47C and FIG. 47B respectively.

FIG. 47E shows in the upper panel a heat map of the surface expression phenotypes (measured as a percentage of the population) of differentiated iNK cells derived from unedited PCS iPSCs when differentiated using NK-MACS or APEL2 methods as depicted in FIG. 47C and FIG. 47B respectively. The bottom panel displays representative histogram plots to illustrate the differences in the iNKs generated by the two methods.

FIG. 47F shows a heat map of the surface expression phenotypes (measured as a percentage of the population) of differentiated edited iNKs (TGFβRII knockout, CISH knockout, and double knockout (DKO)) and unedited parental iPSCs (WT) when differentiated using NK-MACS or APEL2 methods as depicted in FIG. 47C and FIG. 47B respectively.

FIG. 47G shows unedited iNK cell effector function when differentiated using NK-MACS or APEL2 methods as depicted in FIG. 47C and FIG. 47B respectively.

FIG. 48 shows differentiation phenotypes of edited clones (TGFβRII knockout, CISH knockout, and double knockout) as compared to parental wild type clones.

FIG. 49 shows surface expression phenotype of edited iNKs (TGFβRII knockout, CISH knockout, and double knockout) as compared to parental clone iNKs and wild type cells.

FIG. 50A shows surface expression phenotype of edited iNKs (TGFβRII knockout, CISH knockout, and double knockout) as compared to parental clone iNKs (“WT”) and peripheral blood-derived natural killer cells.

FIG. 50B is a flow cytometry histogram plot that shows the surface expression phenotype of edited iNK cells (TGFβRII/CISH double knockout) as compared to parental clone iNK cells (“unedited iNK cells”).

FIG. 50C shows surface expression phenotypes (measured as a percentage of the population) of edited iNK cells (TGFβRII/CISH double knockout) as compared to parental clone iNK cells (“unedited iNK cells”) at day 25, day 32, and day 39 post-hiPSC differentiation (average values from at least 5 separate differentiations).

FIG. 50D shows pSTAT3 expression phenotypes (measured as a percentage of the population) of edited CD56+ iNK cells (“CISH KO iNKs”) as compared to parental clone CD56+ iNK cells (“unedited iNKs”) at 10 minutes and 120 minutes following IL-15 induced activation. Briefly, the day 39 or day 40 iNKs are plated the day before in a cytokine starved condition. The next day the cells are stimulated with 10 ng/ml of IL 15 for the length of time indicated. The cells are fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 50E shows pSMAD2/3 expression phenotypes (measured as a percentage of the population) of edited CD56+ iNK cells (TGFβRII/CISH double knockout, “DKO iNKs”) as compared to parental clone CD56+ iNK cells (“unedited iNK cells”) at 10 minutes and 120 minutes following IL-15 and TGF-β induced activation. Briefly, the day 39 or day 40 iNKs were plated the day before in a cytokine starved condition. The next day the cells were stimulated with 10 ng/ml of IL-15 and 50 ng/ml of TGF-β for the length of time indicated. The cells were fixed immediately at the end of the time point, stained for CD56 followed by an intracellular stain. The cells were processed on a NovoCyte Quanteon and the data was analyzed in FlowJo. Data shown is a representative experiment of >3 experiments performed.

FIG. 50F shows IFN-γ expression phenotypes (measured as a percentage of the population) of edited CD56+ iNK cells (TGFβRII/CISH double knockout, “DKO IFNg”) as compared to parental clone CD56+ iNK cells (unedited iNKs, “WT IFNg”) with or without phorbol myristate acetate (PMA) and ionomycin (IMN) stimulation. The data is representative. It is generated from a single differentiation and each condition in the assay is run with 2 technical replicates. **p<0.05 vs unedited iNK cells (paired t test).

FIG. 50G shows TNF-α expression phenotypes (measured as a percentage of the population) of edited CD56+ iNK cells (TGFβRII/CISH double knockout, “DKO TNF a”) as compared to parental clone CD56+ iNK cells (unedited iNK cells, “WT TNFa”) with or without Phorbol myristate acetate (PMA) and Ionomycin (IMN) stimulation. The data is representative. It is generated from a single differentiation and each condition in the assay is run with 2 technical replicates. **p<0.05 vs unedited iNK cells (paired t test).

FIG. 51A is a schematic representation of an exemplary solid tumor cell killing assay, depicting the use of edited iNK cells (TGFβRII/CISH double knockout) to kill SK-OV-3 ovarian cells in the presence or absence of IL-15 and TGF-β.

FIG. 51B shows the results of a solid tumor killing assay as described in FIG. 51A. iNK cells function to reduce tumor cell spheroid size. Certain edited iNK cells (CISH single knockout, “CISH_2, 4, 5, and 8”) were not significantly different from the parental clone iNK cells (“WT_2”), while certain edited iNK cells (TGFβRII single knockout, “TGFβRII_7”, and TGFβRII/CISH double knockout “DKO”) functioned significantly better at effector-target (E:T) ratios of 1 or greater when measured in the presence of TGF-β as compared to parental clone iNK cells (“WT_2”). ****p<0.0001 vs unedited iNK cells (two-way ANOVA, Sidak's multiple comparisons test).

FIG. 51C shows edited iNK cell effector function as compared to unedited iNK cells.

FIG. 52 shows the results of an in-vitro serial killing assay, where iNK cells are serially challenged with hematological cancer cells (e.g., Nalm6 cells) in the presence of 10 ng/ml of IL-15 and 10 ng/ml of TGF-β; the X axis represents time, with tumor cells being added every 48 hours, while the Y axis represents killing efficacy as measured by normalized total red object area (e.g., presence of tumor cells). The data shows that edited iNK cells (TGFβRII/CISH double knockout) continue to kill hematological cancer cells while unedited iNK cells lose this function at equivalent time points.

FIG. 53 shows surface expression phenotypes (measured as a percentage of the population) of certain edited iNK clonal cells (CISH single knockout “CISH_C2, C4, C5, and C8”, TGFβRII single knockout “TGFβRII-C7”, and TGFβRII/CISH double knockout “DKO-C1”) as compared to parental clone iNK cells (“WT”) at day 25, day 32, and day 39 post-hiPSC differentiation when cultured in the presence of 1 ng/ml or 10 ng/ml IL-15.

FIG. 54A is a schematic of an in-vivo tumor killing assay. Mice were intraperitoneally inoculated with 1×10⁶ SKOV3-luc cells, mice are randomized, and 4 days later, 20×10⁶ iNK cells were introduced intraperitoneally. Mice were followed for up to 60 days post-tumor implantation. The X axis represents time since implantation, while the Y axis represents killing efficacy as measured by total bioluminescence (p/s).

FIG. 54B shows the results of an in-vivo tumor killing assay as described in FIG. 54A. An individual mouse is represented by each horizontal line. The data show that both unedited iNK cells (“unedited iNK”) and DKO edited iNK cells (TGFβRII/CISH double knockout) prevent tumor growth better than vehicle, while edited iNK cells kill tumor cells significantly better than vehicle in-vivo. Each experimental group had 9 animals each. ***p<0.001, ****p<0.0001 by a 2-way ANOVA analysis.

FIG. 54C shows the averaged results with standard error of the mean of the in-vivo tumor killing assay described in FIG. 54B. Populations of mice are represented by each horizontal line. The data show that DKO edited iNK cells (TGFβRII/CISH double knockout) prevent tumor growth and kill tumor cells significantly better than vehicle or unedited iNK cells in-vivo. ***p<0.001, ****p<0.0001 by a 2-way ANOVA analysis.

FIG. 55A shows surface expression phenotypes (measured as a percentage of the population) of bulk edited iNK cells (left panel—ADORA2A single knockout) or certain edited iNK clonal cells (right panel—ADORA2A single knockout) as compared to parental clone iNK cells (“PCS_WT”) at day 25, day 32, and day 39 or at day 28, day 36, and day 39 post-hiPSC differentiation. Representative data from multiple differentiations.

FIG. 55B shows cyclic AMP (cAMP) concentration phenotypes following 5′-(N-Ethylcarboxamido)adenosine (“NECA”, adenosine agonist) activation for edited iNK clonal cells (ADORA2A single knockout) as compared to parental clone iNK cells (“unedited iNKs”). The Y axis represents average cAMP concentration in nM (a proxy for ADORA2A activation), while the X axis represents NECA concentration in nM.

FIG. 55C shows the results of an in-vitro serial killing assay, where iNK cells are serially challenged with hematological cancer cells (e.g., Nalm6 cells) in the presence of 100 μM NECA, and 10 ng/ml of IL-15; the X axis represents time, with tumor cells being added every 48 hours, while the Y axis represents killing efficacy as measured by total red object area (e.g., presence of tumor cells). The data shows that edited iNK cells (“ADORA2A KO INK”) kill hematological cancer cells more effectively than unedited iNK cells (“Ctrl iNK”) under conditions that mimic adenosine suppression.

FIG. 56A shows surface expression phenotypes (measured as a percentage of the population) of certain edited iNK clonal cells (TGFβRII/CISH/ADORA2A triple knockout, “CRA_6” and “CR+A_8”) as compared to parental clone iNK cells (“WT_2”) at day 25, day 32, and day 39 post-hiPSC differentiation. Data is representative of multiple differentiations.

FIG. 56B shows cyclic AMP (cAMP) concentration phenotypes following NECA (adenosine agonist) activation for edited iNK clonal cells (TGFβRII/CISH/ADORA2A triple knockout, “TKO iNKs”) as compared to parental clone iNK cells (“unedited iNKs”). The Y axis represents average cAMP concentration in nM (a proxy for ADORA2A activation), while the X axis represents NECA concentration in nM.

FIG. 56C shows the results of a solid tumor killing assay as described in FIG. 51A without IL-15. iNK cells function to reduce tumor cell spheroid size. The Y axis measures total integrated red object (e.g., presence of tumor cells), while the X axis represents the effector to target (E:T) cell ratio. The edited iNK cells (ADORA2A single knockout “ADORA2A”, TGFβRII/CISH double knockout “DKO”, or TGFβRII/CISH/ADORA2A triple knockout “TKO”) had lower EC50 rates when measured in the presence of TGF-β as compared to parental clone iNK cells (“Control”) (average values from at least 3 separate differentiations).

FIG. 57 shows the results of guide RNA selection assays for the loci TGFβRII, CISH, ADORA2A, TIGIT, and NKG2A utilizing in-vitro editing in iPSCs.

FIG. 58A depicts an exemplary flow cytometry chart for a population of T cells transduced by AAV6 comprising a CD19 CAR cargo targeted for knock-in at GAPDH at 5E4 MOI but without the addition of an RNP.

FIG. 58B depicts an exemplary flow cytometry chart for a population of T cells transduced by AAV6 comprising a CD19 CAR cargo targeted for knock-in at GAPDH at 5E4 MOI and transformed with 1 μM of RNPs comprising Cas12a (SEQ NO: 62) and RSQ22337.

FIG. 58C depicts exemplary expansion and viability data for a population of T cells transduced by AAV6 as described in FIG. 58A and FIG. 58B.

FIG. 58D depicts an exemplary flow cytometry chart for a population of T cells that have been transformed with RNPs targeting the TRAC locus.

FIG. 58E depicts an exemplary flow cytometry chart for a population of T cells transduced by AAV6 comprising a CD19 CAR cargo targeted for knock-in at GAPDH at 5E4 MOI and transformed with 4 μM of RNPs comprising Cas12a (SEQ NO: 62) and RSQ22337 and RNPs targeting the TRAC locus.

FIG. 58F depicts a histogram showing genotype data derived from exemplary flow cytometry experiments on populations of T cells transformed with TRAC targeting RNPs, GAPDH targeting RNPs, and/or transduced with AAV6 comprising a CD19 CAR cargo targeted for knock-in at GAPDH. T cells that have CD19 CAR KI were observed at rates greater than 90% when cells were transformed with GAPDH targeting RNPs and transduced with AAV6 comprising the CD19 CAR cargo targeting GAPDH. T cells that have TRAC KO and CD19 CAR KI were observed at rates greater than 80% when cells were transformed with TRAC targeting RNPs, GAPDH targeting RNPs, and transduced with AAV6 comprising a CD19 CAR cargo targeting GAPDH.

FIG. 58G depicts an exemplary flow cytometry chart for a population of T cells transduced by AAV6 comprising a CD19 CAR cargo targeted for knock-in at GAPDH at 5E4 MOI and transformed with 4 μM of RNPs comprising Cas12a (SEQ NO: 62) with RSQ22337, TRAC targeting RNPs, and TGFBR2 targeting RNPs.

FIG. 58H depicts a histogram showing genotype data derived from exemplary flow cytometry experiments on populations of T cells transformed with GAPDH targeting RNPs (comprising Cas12a (SEQ ID NO: 62) and RSQ22337), and transduced with AAV6 comprising a GFP cargo targeted for knock-in at GAPDH, a CD19 CAR cargo targeted for knock-in at GAPDH, or an HLA-E alloshield cargo targeted for knock-in at GAPDH. Transgene integration efficiencies greater than 80% at the GAPDH locus were observed for each population of edited T cells.

FIG. 58I shows the results of an in-vitro tumor cell killing assay, where T cells comprising CD19 CAR knock-in at the GAPDH gene were challenged with hematological cancer cells (e.g., Raji cells). Significant Raji cell cytolysis was observed in test samples when compared to control samples comprising cancer cells only or when compared to T cells comprising GFP knock-in at the GAPDH gene that were challenged with Raji cells. N=4, 1 biological replicate in 4 technical replicates, shown are the mean and standard error of the mean, statistical analysis with one-way ANOVA provides a P value of <0.0001.

FIG. 58J shows the results of an in-vitro tumor cell killing assay, where T cells comprising CD19 CAR knock-in at the GAPDH gene in combination with TRAC and/or TGFBR2 knock-out were challenged with hematological cancer cells (e.g., Raji cells). As compared to T cells comprising GFP knock-in at the GAPDH gene or unedited T cells, significant cytotoxicity was observed with T cells comprising the CD19 CAR knock-in as assessed by LDH release following 24 hours of co-culture at an E:T of 2. Average spontaneous LDH release by Raji cells (dashed horizontal line) and average LDH released upon treatment with lysis buffer (solid horizontal line) provided for comparison. Each filled circle represents data from four technical replicates from one biological sample. The X axis denotes T cell group, while the Y axis quantifies LDH release as relative fluorescence units (RFUs) as detected using a plate reader with an excitation of 560 nm and emission of 590 nm. Black lines represent means. Not significant (n.s.), ***p<0.001, ****p<0.0001 (unpaired t-test).

FIG. 59 depicts HLA-E surface expression in T cells modified as described herein. Left panel depicts HLA-E surface expression in T cells transduced with AAV6 comprising a B2M-HLA-E cargo targeted for knock-in at GAPDH at 5E4 MOI and transformed with 1 μM of RNPs comprising Cas12a (SEQ NO: 62) with RSQ22337, compared to mock transduced control cells (no AAV6 transduction). Right panel depicts expansion data for T cells comprising knock-in of the B2M-HLA-E cargo at GAPDH and expansion data for the mock transduced control T cells. Cells were stained with PE anti-human HLA-E antibody clone: 3D12 (1:100 dilution).

FIG. 60A is a comparison of T cells modified as described herein utilizing either a one-step or a sequential process, wherein a combination of RNPs targeting different loci are administered to the T cells either together (one step) or sequentially. The left panel depicts exemplary flow cytometry data from T cells that have undergone a one-step electroporation for transformation with RNPs targeting TRAC, B2M, and GAPDH (0.5 μM of each type of RNP) combined with transduction with AAV6 comprising a GFP cargo targeted for knock-in at GAPDH at 5E4 MOI. The right panel depicts exemplary flow cytometry data from T cells that have undergone a series of electroporations for transformation wherein RNPs targeting GAPDH (at 5 μM) were administered to the cells along with transduction with AAV6 comprising a GFP cargo targeted for knock-in at GAPDH at 5E4 MOI, followed four days later by transformation with RNPs targeting TRAC, and RNPs targeting B2M at 0.5 μM of each RNP. Flow cytometry data assayed the number of cells that had at least TRAC knocked-out, the number of cells that had at least B2M knocked-out, and the number of cells that had both TRAC and B2M knocked-out and also exhibited GFP expression. These results show one-step KO/KI has comparable efficiency when compared to sequential KI and KO processes.

FIG. 60B depicts the total number of editing events found in T cells modified as described herein using a one-step process comprising transforming a population of T cells with RNPs targeting TRAC, B2M, CIITA, TGFBR2, and GAPDH (comprising Cas12a (SEQ ID NO: 62) and RSQ22337, and transducing the cells with an AAV6 comprising a GFP cargo targeted for knock-in at the GAPDH gene. Each editing event (KO or cargo KI) occurred at an individual rate of greater than 80%.

FIG. 61A depicts an exemplary flow cytometry chart for a population of NK cells transduced by AAV6 comprising a GFP cargo targeted for knock-in at GAPDH at 5E4 MOI but without the addition of an RNP.

FIG. 61B depicts an exemplary flow cytometry chart for a population of NK cells transduced by AAV6 comprising a GFP cargo targeted for knock-in at GAPDH at 5E4 MOI and transformed with 4 μM of RNPs comprising Cas12a (SEQ NO: 62) and RSQ22337.

FIG. 61C depicts an exemplary flow cytometry chart for a population of NK cells transduced by AAV6 comprising a CD19 CAR cargo targeted for knock-in at GAPDH at 5E4 MOI but without the addition of an RNP.

FIG. 61D depicts an exemplary flow cytometry chart for a population of NK cells transduced by AAV6 comprising a CD19 CAR cargo targeted for knock-in at GAPDH at 5E4 MOI and transformed with 4 μM of RNPs comprising Cas12a (SEQ NO: 62) and RSQ22337.

FIG. 61E depicts a histogram showing genotype data derived from exemplary flow cytometry experiments on populations of NK cells transformed with GAPDH targeting RNPs (comprising Cas12a (SEQ ID NO: 62) and RSQ22337) and transduced with AAV6 comprising either a GFP cargo targeted for knock-in at GAPDH at 5E4 MOI or a CD19 CAR cargo targeted for knock-in at GAPDH. Transgene integration efficiencies greater than 80% at the GAPDH locus were observed in each edited NK cell population.

FIG. 61F shows the results of an in vitro tumor cell killing assay, where NK cells comprising CD19 CAR knock-in at the GAPDH gene were challenged with hematological cancer cells (Raji cells). Significantly greater Raji cell cytolysis was observed in edited NK cells comprising CD19 CAR KI when compared to control NK cells (unedited). N=3, 1 biological replicate in 3 technical replicates, shown are the mean and standard error of the mean, statistical analysis with one-way ANOVA provides a P value of <0.05.

FIG. 61G shows the results of an in vitro tumor killing assay, where NK cells comprising CD19 CAR knock-in (KI) or GFP knock-in (KI) at the GAPDH gene were challenged with hematological cancer cells (Nalm6 cells). Significantly greater cytotoxicity was observed with NK cells comprising the CD19 CAR knock-in than the GFP knock-in as assessed by BATDA release following 2 hours of co-culture at an E:T of 1. Average spontaneous BATDA release by Nalm6 cells (dashed horizontal line) and average BATDA released upon treatment with lysis buffer (solid horizontal line) provided for comparison. Each filled circle represents data from eight technical replicates from one biological sample. The X axis denotes NK cell group, while the Y axis quantifies BATDA release as relative fluorescence units (RFUs) as detected by a time-resolved fluorometer. Black horizontal lines represent means. ****p<0.0001 (unpaired t-test).

FIG. 62A shows the results of an in vitro persistence assay of mbIL-15/CD16 (CD16^{+/+}/mbIL-15^{+/+}) DKI/CISH/TGFβRII DKO (DKI/DKO) iNK cells and unedited (WT) iNK cells. The X axis represents days since removal of exogenous cytokine support, while the Y axis represents the total number of live cells.

FIG. 62B shows averaged results of an in vitro persistence assay of mbIL-15/CD16 (CD16^{+/+}/mbIL-15^{+/+}) DKI/CISH/TGFβRII DKO (DKI/DKO) iNK cells and CD16/mbIL-15 DKI (DKI) iNK cells. The X axis represents days since removal of exogenous cytokine support, while the Y axis represents the total number of live cells.

FIG. 63A shows averaged results of an in vitro tumor cell killing assay where mbIL-15/CD16 (CD16^{+/+}/mbIL-15^{+/+}) DKI/CISH/TGFβRII DKO (DKI/DKO) iNK cells with or without 10 μg/ml cetuximab (CTX) were added to Detroit-562 (pharyngeal carcinoma) cells at various E:T ratios (e.g., 1:1, 5:1, 10:1). The X axis represents time in hours:minutes:seconds from initial seeding of the Detroit-562 cells, while the Y axis represents percent cytolysis as measured by electrical impedance. N=3, error bars represent standard deviation.

FIG. 63B shows averaged results of an in vitro tumor cell killing assay where mbIL-15/CD16 (CD16^{+/+}/mbIL-15^{+/+}) DKI/CISH/TGFβRII DKO (DKI/DKO) iNK cells with or without 10 μg/ml cetuximab (CTX) were added to FaDu (pharyngeal carcinoma) cells at various E:T ratios (e.g., 1:1, 5:1, 10:1). The X axis represents time in hours:minutes:seconds from initial seeding of the FaDu cells, while the Y axis represents percent cytolysis as measured by electrical impedance. N=3, error bars represent standard deviation.

FIG. 63C shows averaged results of an in vitro tumor cell killing assay where mbIL-15/CD16 (CD16^{+/+}/mbIL-15^{+/+}) DKI/CISH/TGFβRII DKO (DKI/DKO) iNK cells with or without 10 μg/ml cetuximab (CTX) were added to HT29 (colorectal adenocarcinoma) cells at various E:T ratios (e.g., 1:1, 5:1, 10:1). The X axis represents time in hours:minutes:seconds from initial seeding of the HT29 cells, while the Y axis represents percent cytolysis as measured by electrical impedance. N=3, error bars represent standard deviation.

FIG. 63D shows averaged results of an in vitro tumor cell killing assay where mbIL-15/CD16 (CD16^{+/+}/mbIL-15^{+/-}) DKI/CISH/TGFβRII DKO (DKI/DKO) iNK cells with or without 10 μg/ml cetuximab (CTX) were added to HCT116 (colorectal carcinoma) cells at various E:T ratios (e.g., 1:1, 5:1, 10:1). The X axis represents time in hours:minutes:seconds from initial seeding of the HCT116 cells, while the Y axis represents percent cytolysis as measured by electrical impedance. N=3, error bars represent standard deviation.

FIG. 64A shows averaged results of an in vitro tumor cell killing assay where mbIL-15/CD16 (CD16^{+/+}/mbIL-15^{+/+}) DKI/CISH/TGFβRII DKO (DKI/DKO) iNK cells or unedited (WT) iNK cells added to HT29 (colorectal adenocarcinoma) cells at an E:T ratio of 10:1. The X axis represents time in hours:minutes:seconds from initial seeding of the HT29 cells, while the Y axis represents percent cytolysis as measured by electrical impedance. N=3, error bars represent standard deviation.

FIG. 64B shows results of an in vitro persistence assay of mbIL-15/CD16 (CD16^{+/+}/mbIL-15^{+/+}) DKI/CISH/TGFβRII DKO (DKI/DKO) iNK cells and unedited (WT) iNK cells. DKI/DKO or WT iNK cells were co-cultured with HT-29 cells for 4 days at a 10:1 E:T ratio. The X axis denotes evaluation category (e.g., percentage of live NK cells of all cells, percentage of CD16+ live NK cells), while the Y axis represents the percentage as measured by flow cytometry. Black horizontal lines represent means.

FIG. 64C depicts exemplary flow cytometry data from before and after an in vitro persistence assay of mbIL-15/CD16 (CD16^{+/+}/mbIL-15^{+/+}) DKI/CISH/TGFβRII DKO (DKI/DKO) iNK cells and unedited (WT) iNK cells. DKI/DKO or WT iNK cells were co-cultured with HT-29 cells for 4 days at a 1:1 E:T ratio.

FIG. 65A shows exemplary flow cytometry data from unedited (WT) iNK cells or mbIL-15/CD16 (CD16^{+/+}/mbIL-15^{+/+}) DKI/CISH/TGFβRII DKO (DKI/DKO) iNK cells. The data highlights the efficiency of integration and expression of knock-in cassettes comprising a CD16 and IL-15Rα protein encoding cargo sequence. The X axis denotes whether the selected gene is CD16 or IL-15Rα, while the Y axis quantifies the percentage of cells from the noted population that are expressing the selected gene. Horizontal lines represent group means. N=1, ****p<0.0001 (two-way ANOVA).

FIG. 65B shows the results of 3D tumor spheroid killing assays conducted as depicted in FIG. 20. Unedited (WT) iNK cells or mbIL-15/CD16 (CD16^{+/+}/mbIL-15^{+/+}) DKI/CISH/TGFβRII DKO (DKI/DKO) iNK cells were used against SK-OV-3 tumor cells at varying E:T ratios. DKI/DKO or WT iNK cells were co-cultured with the tumor spheroids and imaged every 2 hours to measure red object intensity (a proxy for tumor cell abundance) for up to 4 days. Data were normalized to the red object intensity at time of iNK cell addition. IC50 values based on the left panel are presented in the table in the right panel and highlight the greater efficacy of the DKI/DKO iNK cells in killing tumor cells. The X axis represents time in hours since addition of iNK cells to the tumor spheroid, while the Y axis represents normalized spheroid size as measured by red object intensity. N=1, two technical replicates per cell line.

FIG. 65C shows the results of a 3D tumor spheroid killing assay conducted as depicted in FIG. 20. Unedited (WT) iNK cells or mbIL-15/CD16 (CD16^{+/+}/mbIL-15^{+/+}) DKI/CISH/TGFβRII DKO (DKI/DKO) iNK cells were used against SK-OV-3 tumor cells at varying E:T ratios and in the presence of either 10 μg/ml trastuzumab or IgG (control). DKI/DKO or WT iNK cells were co-cultured with the tumor spheroids and imaged every 2 hours to measure red object intensity (a proxy for tumor cell abundance) for up to 4 days. DKI/DKO iNK cells demonstrate significantly greater antibody-dependent cellular cytotoxicity (ADCC) than WT iNK cells. The X axis represents treatment group, while the Y axis represents the calculated IC50 (e.g., the E:T ratio required to reduce the SK-OV-3 spheroids by 50% after 100 hours of killing). Data represents 11 independent experiments. ****p<0.0001 (unpaired t-test).

FIG. 65D shows the results of an in vitro persistence assay of unedited (WT) iNK cells and mbIL-15/CD16 (CD16^{+/+}/mbIL-15^{+/+}) DKI/CISH/TGFβRII DKO (DKI/DKO) INK cells in the absence of the cytokines IL-2 and IL-15. The X axis represents days in culture since removal of exogenous cytokine support, while the Y axis represents viability as the percentage of live cells. N=1, two technical replicates per cell line, error bars represent standard deviation.

FIG. 65E shows the results of an in vitro SMAD2/3 phosphorylation assay of unedited (WT) iNK cells and mbIL-15/CD16 (CD16^{+/+}/mbIL-15^{+/+}) DKI/CISH/TGFβRII DKO (DKI/DKO) iNK cells following treatment with TGFβ (TGFb). DKI/DKO iNK cells or WT iNK cells were plated in a cytokine starved condition and 10 ng/ml of TGFβ was added to the iNK cells the following day. Cells were immediately fixed following the time indicated. The X axis represents time in minutes since addition of the TGFB, while the Y axis represents normalized level of SMAD2/3 phosphorylation. Data represents one independent experiment. Dashed horizontal line represents level of SMAD2/3 phosphorylation following treatment with vehicle.

FIG. 65F shows the results of a 3D tumor spheroid killing assay conducted as depicted in FIG. 20. Unedited (WT) iNK cells or mbIL-15/CD16 (CD16^{+/+}/mbIL-15^{+/+}) DKI/CISH/TGFβRII DKO (DKI/DKO) iNK cells were used against SK-OV-3 tumor cells at an E:T ratio of 31.6 and in the presence of either 10 ng/ml TGFβ or IgG (control). DKI/DKO or WT iNK cells were co-cultured with the tumor spheroids and imaged every 2 hours to measure red object intensity (a proxy for tumor cell abundance) for up to 100 days. Results for the DKI/DKO iNK cells are displayed in the left panel, while the results for the WT iNK cells are displayed in the right panel. The X axis represents time in hours since addition of iNK cells to the tumor spheroid, while the Y axis represents normalized spheroid size as measured by red object intensity. N=1.

FIG. 65G shows the results of an in vitro serial killing assay where unedited (WT) iNK cells or mbIL-15/CD16 (CD16^{+/+}/mbIL-15^{+/+}) DKI/CISH/TGFβRII DKO (DKI/DKO) INK cells were challenged with Nalm6 tumor cells. At day 0, 10×10³ Nalm6 tumor cells and 2×10⁵ iNK cells were plated together in the presence of 10 ng/ml TGFB. At 48 hour intervals, a bolus of 5×10³ Nalm6 tumor cells was added to re-challenge the iNK cell population. The X axis represents the number of challenges that occurred, while the Y axis represents the tumor burden as measured by red object intensity. N=1, three technical replicates per cell line, error bars represent standard deviation.

FIG. 66A is a schematic of an in vivo tumor killing assay. Mice were intravenously (IV) inoculated with 0.125×10⁶ (0.125e6) SKOV3-luc cells, and following 19 days to allow for tumor establishment, on day −2, mice were imaged to establish pre-treatment tumor burden and randomized into two groups. Two days later, on day 0, a first group of mice intravenously received 20×10⁶ (20e6) mbIL-15/CD16 (CD16^{+/+}/mbIL-15^{-/-}) DKI/CISH/TGFβRII DKO (DKI/DKO) iNK cells in combination with 2.5 mpk trastuzumab (Tras) and a second group of mice intraperitoneally received only 2.5 mpk trastuzumab (Tras). Mice were imaged weekly using an in vivo imaging system (IVIS) to assess tumor burden over time.

FIG. 66B shows tumor burden (median with interquartile range) for the in vivo tumor killing assay described in FIG. 66A. Groups of mice are represented by each horizontal line. Each treatment group had 4 mice. The groups include mice that received mbIL-15/CD16 (CD16^{+/+}/mbIL-15^{+/+}) DKI/CISH/TGFβRII DKO (DKI/DKO) iNK cells in combination with a single dose of trastuzumab (DKI/DKO iNK+Tras.), a single dose of trastuzumab alone (Tras. Only), or an isotype control. Mice dosed with the DKI/DKO iNK cells in combination with trastuzumab had significantly decreased tumor burden as compared to mice dosed with trastuzumab alone. The dose of trastuzumab on day 0 is indicated by the arrow. The dashed vertical line represents the dose of iNK cells. The X axis represents time in days since introduction of NK cells, while the Y axis represents tumor burden as measured by bioluminescent imaging (BLI) using an in vivo imaging system (IVIS).

FIG. 66C shows representative bioluminescent imaging of mice subjected to the in vivo tumor killing assay described in FIG. 66A. The treatment groups of the mice are denoted along the top of the panel, while the time since dosing with iNK cells in combination with trastuzumab or trastuzumab alone is denoted along the left side of the panel. Each treatment group had 4 mice. The color scale at the right represents the radiance (p/sec/cm²/sr) of the bioluminescence (from a minimum of 3.94×10⁴ to a maximum of 7.02×10⁵) as seen in the images.

FIG. 67A is a schematic of an in vivo tumor killing assay. Mice were intraperitoneally inoculated with 0.25×10⁶ SKOV3-luc cells, and following 4 days to allow for tumor establishment, mice were randomized into groups. One day later, some groups of mice intraperitoneally received 5×10⁶ (5E6) unedited (WT) or mbIL-15/CD16 (CD16^{+/+}/mbIL-15^{+/+}) DKI/CISH/TGFβRII DKO (DKI/DKO) iNK cells. In some treatment groups, mice received a dose of 2.5 mpk trastuzumab at 0, 7, and 14 days (as indicated by the arrows) post-introduction of iNK cells, for a total of 3 doses of trastuzumab. Mice were imaged weekly using an in vivo imaging system (IVIS) to assess tumor burden over time.

FIG. 67B shows tumor burden (median with interquartile range) for the in vivo tumor killing assay described in FIG. 67A. Groups of mice are represented by each horizontal line. Each treatment group had 5-6 mice. The groups included mice that received unedited iNK cells (WT iNK), mbIL-15/CD16 (CD16^{+/+}/mbIL-15^{+/+}) DKI/CISH/TGFβRII DKO INK cells (DKI/DKO iNK), or an isotype control. The X axis represents time since introduction of NK cells, while the Y axis represents tumor burden as measured by bioluminescent imaging (BLI) using an in vivo imaging system (IVIS).

FIG. 67C shows tumor burden (median with interquartile range) for the in vivo tumor killing assay described in FIG. 67A. Groups of mice are represented by each horizontal line. Each treatment group had 5-6 mice. The groups included mice that received unedited (WT) iNK cells in combination with trastuzumab (WT+Tras.×3), mbIL-15/CD16 (CD16^{+/+}/mbIL-15^{+/+}) DKI/CISH/TGFβRII DKO (DKI/DKO) iNK cells in combination with trastuzumab (DKI DKO+Tras.×3), trastuzumab alone, or an isotype control. Mice dosed with the DKI/DKO iNK cells in combination with trastuzumab had significantly decreased tumor burden as compared to mice dosed with WT iNK cells in combination with trastuzumab or trastuzumab alone. Doses of trastuzumab on day 0, 7, and 14 are indicated by the arrows. The X axis represents time since introduction of NK cells, while the Y axis represents tumor burden as measured by bioluminescent imaging (BLI) using an in vivo imaging system (IVIS). ****p<0.0001 (one-way ANOVA).

FIG. 67D shows the survival of mice subjected to the in vivo tumor killing assay described in FIG. 67A. Groups of mice are represented by each horizontal line. The X axis represents time since introduction of NK cells, while the Y axis represents percent survival of the mice. *p<0.05, **p<0.01 (Log-rank Mantel-Cox test).

FIG. 67E shows representative bioluminescent imaging of mice subjected to the in vivo tumor killing assay described in FIG. 67A. The treatment groups of the mice are denoted along the top of the panel, while the time since introduction of NK cells is denoted along the left side of the panel. Each treatment group had 5-6 mice. The table below the images displays the number of mice with complete tumor clearance/total mice in the treatment group (from top of panel) at day 31 post-introduction of NK cells.

### DETAILED DESCRIPTION

### Definitions and Abbreviations

Unless otherwise specified, each of the following terms have the meaning set forth in this section.

The indefinite articles “a” and “an” refer to at least one of the associated noun, and are used interchangeably with the terms “at least one” and “one or more.” The conjunctions “or” and “and/or” are used interchangeably as non-exclusive disjunctions.

The term “cancer” (also used interchangeably with the term “neoplastic”), as used herein, refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair.

The terms “CRISPR/Cas nuclease” as used herein refer to any CRISPR/Cas protein with DNA nuclease activity, e.g., a Cas9 or a Cas12 protein that exhibits specific association (or “targeting”) to a DNA target site, e.g., within a genomic sequence in a cell in the presence of a guide molecule. The strategies, systems, and methods disclosed herein can use any combination of CRISPR/Cas nuclease disclosed herein, or known to those of ordinary skill in the art. Those of ordinary skill in the art will be aware of additional CRISPR/Cas nucleases and variants suitable for use in the context of the present disclosure, and it will be understood that the present disclosure is not limited in this respect.

The term “differentiation” as used herein is the process by which an unspecialized (“uncommitted”) or less specialized cell acquires the features of a specialized cell such as, for example, a blood cell. In some embodiments, a differentiated or differentiation-induced cell is one that has taken on a more specialized (“committed”) position within the lineage of a cell. For example, an iPS cell (iPSC) can be differentiated into various more differentiated cell types, for example, a hematopoietic stem cell, a lymphocyte, and other cell types, upon treatment with suitable differentiation factors in the cell culture medium. Suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

The terms “differentiation marker,” “differentiation marker gene,” or “differentiation gene,” as used herein refers to genes or proteins whose expression are indicative of cell differentiation occurring within a cell, such as a pluripotent cell. In some embodiments, differentiation marker genes include, but are not limited to, the following genes: CD34, CD4, CD8, CD3, CD56 (NCAM), CD49, CD45, NK cell receptor (cluster of differentiation 16 (CD16)), natural killer group-2 member D (NKG2D), CD69, NKp30, NKp44, NKp46, CD158b, FOXA2, FGF5, SOX17, XIST, NODAL, COL3A1, OTX2, DUSP6, EOMES, NR2F2, NROB1, CXCR4, CYP2B6, GAT A3, GATA4, ERBB4, GATA6, HOXC6, INHA, SMAD6, RORA, NIPBL, TNFSF11, CDH11, ZIC4, GAL, SOX3, PITX2, APOA2, CXCL5, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CDID FOXG1, LEFTY1, TUJI, T gene (Brachyury), ZIC1, GATA1, GATA2, HDAC4, HDAC5, HDAC7, HDAC9, NOTCHI, NOTCH2, NOTCH4, PAX5, RBPJ, RUNX1, STAT1 and STAT3.

The terms “differentiation marker gene profile,” or “differentiation gene profile,” “differentiation gene expression profile,” “differentiation gene expression signature,” “differentiation gene expression panel,” “differentiation gene panel,” or “differentiation gene signature” as used herein refer to expression or levels of expression of a plurality of differentiation marker genes.

The term “nuclease” as used herein refers to any protein that catalyzes the cleavage of phosphodiester bonds. In some embodiments the nuclease is a DNA nuclease. In some embodiments the nuclease is a “nickase” which causes a single-strand break when it cleaves double-stranded DNA, e.g., genomic DNA in a cell. In some embodiments the nuclease causes a double-strand break when it cleaves double-stranded DNA, e.g., genomic DNA in a cell. In some embodiments the nuclease binds a specific target site within the double-stranded DNA that overlaps with or is adjacent to the location of the resulting break. In some embodiments, the nuclease causes a double-strand break that contains overhangs ranging from 0 (blunt ends) to 22 nucleotides in both 3′ and 5′ orientations. As discussed herein, CRISPR/Cas nucleases, zinc finger nucleases (ZFNs), transcription activator-like effector nucleases (TALENs) and meganucleases are exemplary nucleases that can be used in accordance with the strategies, systems, and methods of the present disclosure.

The term “edited iNK cell” as used herein refers to an iNK cell which has been modified to change at least one expression product of at least one gene at some point in the development of the cell. In some embodiments, a modification can be introduced using, e.g., gene editing techniques such as CRISPR-Cas or, e.g., dominant-negative constructs. In some embodiments, an iNK cell is edited at a time point before it has differentiated into an iNK cell, e.g., at a precursor stage, at a stem cell stage, etc. In some embodiments, an edited iNK cell is compared to a non-edited iNK cell (an NK cell produced by differentiating an iPSC cell, which iPSC cell and/or iNK cell do not have modifications, e.g., genetic modifications).

The term “embryonic stem cell” as used herein refers to pluripotent stem cells derived from the inner cell mass of the embryonic blastocyst. In some embodiments, embryonic stem cells are pluripotent and give rise during development to all derivatives of the three primary germ layers: ectoderm, endoderm and mesoderm. In some such embodiments, embryonic stem cells do not contribute to the extra-embryonic membranes or the placenta, i.e., are not totipotent.

The term “endogenous,” as used herein in the context of nucleic acids refers to a native nucleic acid (e.g., a gene, a protein coding sequence) in its natural location, e.g., within the genome of a cell.

The term “essential gene” as used herein with respect to a cell refers to a gene that encodes at least one gene product that is required for survival and/or proliferation of the cell. An essential gene can be a housekeeping gene that is essential for survival of all cell types or a gene that is required to be expressed in a specific cell type for survival and/or proliferation under particular culture conditions, e.g., for proper differentiation of iPS or ES cells or expansion of iPS- or ES-derived cells. Loss of function of an essential gene results, in some embodiments, in a significant reduction of cell survival, e.g., of the time a cell characterized by a loss of function of an essential gene survives as compared to a cell of the same cell type but without a loss of function of the same essential gene. In some embodiments, loss of function of an essential gene results in the death of the affected cell. In some embodiments, loss of function of an essential gene results in a significant reduction of cell proliferation, e.g., in the ability of a cell to divide, which can manifest in a significant time period the cell requires to complete a cell cycle, or, in some preferred embodiments, in a loss of a cell's ability to complete a cell cycle, and thus to proliferate at all.

The term “exogenous,” as used herein in the context of nucleic acids refers to a nucleic acid (whether native or non-native) that has been artificially introduced into a man-made construct (e.g., a knock-in cassette, or a donor template) or into the genome of a cell using, for example, gene editing or genetic engineering techniques, e.g., HDR based integration techniques.

The term “genome editing system” refers to any system having RNA-guided DNA editing activity.

The term “guide molecule” or “guide RNA” or “gRNA” when used in reference to a CRISPR/Cas system is any nucleic acid that promotes the specific association (or “targeting”) of a CRISPR/Cas nuclease, e.g., a Cas9 or a Cas12 protein to a DNA target site such as within a genomic sequence in a cell. While guide molecules are typically RNA molecules it is well known in the art that chemically modified RNA molecules including DNA/RNA hybrid molecules can be used as guide molecules.

The terms “hematopoietic stem cell,” or “definitive hematopoietic stem cell” as used herein, refer to CD34-positive (CD34+) stem cells. In some embodiments, CD34-positive stem cells are capable of giving rise to mature myeloid and/or lymphoid cell types. In some embodiments, the myeloid and/or lymphoid cell types include, for example, T cells, natural killer (NK) cells and/or B cells.

The terms “induced pluripotent stem cell”, “iPS cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

The terms “iPS-derived NK cell” or “INK cell” or as used herein refers to a natural killer cell which has been produced by differentiating an iPS cell, which iPS cell may or may not have a genetic modification.

The terms “iPS-derived T cell” or “iT cell” or as used herein refers to a T which has been produced by differentiating an iPS cell, which iPS cell may or may not have a genetic modification.

The term “multipotent stem cell” as used herein refers to a cell that has the developmental potential to differentiate into cells of one or more germ layers (ectoderm, mesoderm and endoderm), but not all three germ layers. Thus, in some embodiments, a multipotent cell may also be termed a “partially differentiated cell.” Multipotent cells are well-known in the art, and examples of multipotent cells include adult stem cells, such as for example, hematopoietic stem cells and neural stem cells. In some embodiments, “multipotent” indicates that a cell may form many types of cells in a given lineage, but not cells of other lineages. For example, a multipotent hematopoietic cell can form the many different types of blood cells (red, white, platelets, etc.), but it cannot form neurons. Accordingly, in some embodiments, “multipotency” refers to a state of a cell with a degree of developmental potential that is less than totipotent and pluripotent.

The term “pluripotent” as used herein refers to ability of a cell to form all lineages of the body or soma (i.e., the embryo proper) or a given organism (e.g., human). For example, embryonic stem cells are a type of pluripotent stem cells that are able to form cells from each of the three germ layers, the ectoderm, the mesoderm, and the endoderm. Generally, pluripotency may be described as a continuum of developmental potencies ranging from an incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell or an induced pluripotent stem cell).

The term “pluripotency” as used herein refers to a cell that has the developmental potential to differentiate into cells of all three germ layers (ectoderm, mesoderm, and endoderm). In some embodiments, pluripotency can be determined, in part, by assessing pluripotency characteristics of the cells. In some embodiments, pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4 (also known as POU5F1), NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

The term “pluripotent stem cell morphology” as used herein refers to the classical morphological features of an embryonic stem cell. In some embodiments, normal embryonic stem cell morphology is characterized as small and round in shape, with a high nucleus-to-cytoplasm ratio, the notable presence of nucleoli, and typical intercell spacing.

The term “polycistronic” or “multicistronic” when used herein with reference to a knock-in cassette refers to the fact that the knock-in cassette can express two or more proteins from the same mRNA transcript. Similarly, a “bicistronic” knock-in cassette is a knock-in cassette that can express two proteins from the same mRNA transcript.

The term “polynucleotide” (including, but not limited to “nucleotide sequence”, “nucleic acid”, “nucleic acid molecule”, “nucleic acid sequence”, and “oligonucleotide”) as used herein refers to a series of nucleotide bases (also called “nucleotides”) and means any chain of two or more nucleotides. In some embodiments, polynucleotides, nucleotide sequences, nucleic acids, etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids contain modified bases.

Conventional IUPAC notation is used in nucleotide sequences presented herein, as shown in Table 1, below (see also Cornish-Bowden, Nucleic Acids Res. 1985; 13(9):3021-30, incorporated by reference herein). It should be noted, however, that “T” denotes “Thymine or Uracil” in those instances where a sequence may be encoded by either DNA or RNA, for example in certain CRISPR/Cas guide molecule targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “potency” or “developmental potency” as used herein refer to the sum of all developmental options accessible to the cell (i.e., the developmental potency), particularly, for example in the context of cellular developmental potential. In some embodiments, the continuum of cell potency includes, but is not limited to, totipotent cells, pluripotent cells, multipotent cells, oligopotent cells, unipotent cells, and terminally differentiated cells.

The terms “prevent,” “preventing,” and “prevention” as used herein with reference to a disease refer to the prevention of the disease in a mammal, e.g., in a human, including (a) avoiding or precluding the disease; (b) affecting the predisposition toward the disease; or (c) preventing or delaying the onset of at least one symptom of the disease.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The term “gene product of interest” as used herein can refer to any product encoded by a gene including any polynucleotide or polypeptide. In some embodiments the gene product is a protein which is not naturally expressed by a target cell of the present disclosure. In some embodiments the gene product is a protein which confers a new therapeutic activity to the cell such as, but not limited to, a chimeric antigen receptor (CAR) or antigen-binding fragment thereof, a T cell receptor or antigen-binding portion thereof, a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof. It is to be understood that the methods and cells of the present disclosure are not limited to any particular gene product of interest and that the selection of a gene product of interest will depend on the type of cell and ultimate use of the cells.

The term “reporter gene” as used herein refers to an exogenous gene that has been introduced into a cell, e.g., integrated into the genome of the cell, that confers a trait suitable for artificial selection. Common reporter genes are fluorescent reporter genes that encode a fluorescent protein, e.g., green fluorescent protein (GFP) and antibiotic resistance genes that confer antibiotic resistance to cells.

The terms “reprogramming” or “dedifferentiation” or “increasing cell potency” or “increasing developmental potency” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

The terms “RNA-guided nuclease” and “RNA-guided nuclease molecule” are used interchangeably herein. In some embodiments, the RNA-guided nuclease is a RNA-guided DNA endonuclease enzyme. In some embodiments, the RNA-guided nuclease is a CRISPR nuclease. Non-limiting examples of RNA-guided nucleases are listed in Table 5 below, and the methods and compositions disclosed herein can use any combination of RNA-guided nucleases disclosed herein, or known to those of ordinary skill in the art. Those of ordinary skill in the art will be aware of additional nucleases and nuclease variants suitable for use in the context of the present disclosure, and it will be understood that the present disclosure is not limited in this respect.

Additional suitable RNA-guided nucleases, e.g., Cas9 and Cas12 nucleases, will be apparent to the skilled artisan in view of the present disclosure, and the disclosure is not limited by the exemplary suitable nucleases provided herein. In some embodiments, a suitable nuclease is a Cas12a, Cas9, Cas12b, Cas12c, Cas12e, CasX, or CasΦ (Cas12j), or a variant thereof (e.g., a variant with a high editing efficiency, e.g., capable of editing about 60% to 100% of cells in a population of cells) nuclease. In some embodiments, the disclosure also embraces nuclease variants, e.g., Cas9, Cpf1 (Cas12a, such as the Mad7 Cas12a variant), Cas12b, Cas12e, CasX, or CasΦ (Cas12j) nuclease variants. In some embodiments, a nuclease is a nuclease variant, which refers to a nuclease comprising an amino acid sequence characterized by one or more amino acid substitutions, deletions, or additions as compared to the wild type amino acid sequence of the nuclease. In some embodiments, a suitable nuclease and/or nuclease variant may also include purification tags (e.g., polyhistidine tags) and/or signaling peptides, e.g., comprising or consisting of a nuclear localization signal sequence. Some non-limiting examples of suitable nucleases and nuclease variants are described in more detail elsewhere herein and also include those described in PCT application PCT/US2019/22374, filed Mar. 14, 2019, and entitled “Systems and Methods for the Treatment of Hemoglobinopathies,” the entire contents of which are incorporated herein by reference. In some embodiments, the RNA-guided nuclease is an Acidaminococcus sp. Cpf1 variant (AsCpf1 variant). In some embodiments, suitable Cpf1 nuclease variants, including suitable AsCpf1 variants will be known or apparent to those of ordinary skill in the art based on the present disclosure, and include, but are not limited to, the Cpf1 variants disclosed herein or otherwise known in the art. For example, in some embodiments, the RNA-guided nuclease is a Acidaminococcus sp. Cpf1 RR variant (AsCpf1-RR). In another embodiment, the RNA-guided nuclease is a Cpf1 RVR variant. For example, suitable Cpf1 variants include those having an M537R substitution, an H800A substitution, and/or an F870L substitution, or any combination thereof (numbering scheme according to AsCpf1 wild-type sequence).

The term “subject” as used herein means a human or non-human animal. In some embodiments a human subject can be any age (e.g., a fetus, infant, child, young adult, or adult). In some embodiments a human subject may be at risk of or suffer from a disease, or may be in need of alteration of a gene or a combination of specific genes. Alternatively, in some embodiments, a subject may be a non-human animal, which may include, but is not limited to, a mammal. In some embodiments, a non-human animal is a non-human primate, a rodent (e.g., a mouse, rat, hamster, guinea pig, etc.), a rabbit, a dog, a cat, and so on. In certain embodiments of this disclosure, the non-human animal subject is livestock, e.g., a cow, a horse, a sheep, a goat, etc. In certain embodiments, the non-human animal subject is poultry, e.g., a chicken, a turkey, a duck, etc.

The terms “treatment,” “treat,” and “treating,” as used herein refer to a clinical intervention aimed to reverse, alleviate, delay the onset of, or inhibit the progress, ameliorate, reduce severity of, prevent or delay the recurrence of a disease, disorder, or condition or one or more symptoms thereof, and/or improve one or more symptoms of a disease, disorder, or condition as described herein. In some embodiments, a condition includes an injury. In some embodiments, an injury may be acute or chronic (e.g., tissue damage from an underlying disease or disorder that causes, e.g., secondary damage such as tissue injury). In some embodiments, treatment, e.g., in the form of an iPSC-derived NK cell or a population of iPSC-derived NK cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, in some embodiments, treatment may be administered to a susceptible subject prior to the onset of symptoms (e.g., in light of genetic or other susceptibility factors). In some embodiments, treatment may also be continued after symptoms have resolved, for example to prevent or delay their recurrence. In some embodiments, treatment results in improvement and/or resolution of one or more symptoms of a disease, disorder or condition.

The term “variant” as used herein refers to an entity such as a polypeptide or polynucleotide that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As used herein, the terms “functional variant” refer to a variant that confers the same function as the reference entity, e.g., a functional variant of a gene product of an essential gene is a variant that promotes the survival and/or proliferation of a cell. It is to be understood that a functional variant need not be functionally equivalent to the reference entity as long as it confers the same function as the reference entity.

### Target Cells

Methods of the disclosure can be used to edit the genome of any cell. In certain embodiments, the target cell is a stem cell, e.g., an iPS or ES cell. In certain embodiments, the target cell can be an iPS- or ES-derived cell, where the genetic modification is made at any stage during the reprogramming process from donor cell to iPSC, during the iPSC stage, and/or at any stage of the process of differentiating the iPSC or ESC to a specialized cell, or even up to or at the final specialized cell state. In certain embodiments, the target cell can be an iPS-derived NK cell (iNK cell) or iPS-derived T cell (iT cell) where the genetic modification is made at any stage during the reprogramming process from donor cell to iPSC, during the iPSC stage, and/or at any stage of the process of differentiating the iPSC to an iNK or iT state, e.g., at an intermediary state, such as, for example, an iPSC-derived HSC state, or even up to or at the final iNK or iT cell state.

In certain embodiments, a target cell is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In some embodiments, a target cell is a neuronal progenitor cell. In some embodiments, a target cell is a neuron.

In some embodiments, a target cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a target cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a target cell is one or more of a myeloid progenitor cell (e.g., a common myeloid progenitor (CMP) cell or granulocyte macrophage progenitor (GMP) cell). In some embodiments, a target cell is a lymphoid progenitor cell, e.g., a common lymphoid progenitor (CLP) cell. In some embodiments, a target cell is one or more of an erythroid progenitor cell (e.g., an MEP cell). In some embodiments, a target cell is one or more of a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺CD90⁺ cell, CD34⁺CD38⁻ cell, CD34⁺CD90⁺CD49f⁺CD38⁺CD45RA⁻ cell, CD105⁺ cell, CD31⁺, or CD133⁺ cell, or a CD34⁺CD90⁺ CD133⁺ cell. In some embodiments, a target cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a target cell is one or more of a mobilized peripheral blood hematopoietic CD34⁺ cell (after the subject is treated with a mobilization agent, e.g., G-CSF or Plerixafor). In some embodiments, a target cell is a peripheral blood endothelial cell. In some embodiments, a target cell is a peripheral blood natural killer cell.

In certain embodiments, a target cell is a primary cell, e.g., a cell isolated from a human subject. In certain embodiments, a target cell is an immune cell, e.g., a primary immune cell isolated from a human subject. In certain embodiments, a target cell is part of a population of cells isolated from a subject, e.g., a human subject. In some embodiments, the population of cells comprises a population of immune cells isolated from a subject. In some embodiments, the population of cells comprises tumor infiltrating lymphocytes (TILs), e.g., TILs isolated from a human subject. In some embodiments, a target cell is isolated from a healthy subject, e.g., a healthy human donor. In some embodiments, a target cell is isolated from a subject having a disease or illness, e.g., a human patient in need of a treatment.

In certain embodiments, a target cell is an immune cell, e.g., a primary immune cell, e.g., a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, or a CD4⁺ CD25⁺ CD127⁻Foxp3⁺ T cell. In some embodiments, a target cell is an alpha-beta T cell, a gamma-delta T cell or a Treg. In some embodiments a target cell is macrophage. In some embodiments, a target cell is an innate lymphoid cell. In some embodiments, a target cell is a dendritic cell. In some embodiments, a target cell is a beta cell, e.g., a pancreatic beta cell.

In some embodiments, a target cell is isolated from a subject having a cancer.

In some embodiments, a target cell is isolated from a subject having a cancer, including but not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma, medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer); hematopoietic cancer (e.g., lymphomas, primary pulmonary lymphomas, bronchus-associated lymphoid tissue lymphomas, splenic lymphomas, nodal marginal zone lymphomas, pediatric B cell non-Hodgkin lymphomas); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); melanoma; midline tract carcinoma; multiple endocrine neoplasia syndrome; muscle cancer; mesothelioma; nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; vulvar cancer (e.g., Paget's disease of the vulva), or any combination thereof.

In some embodiments, a target cell is isolated from a subject having a hematological disorder. In some embodiments, a target cell is isolated form a subject having sickle cell anemia. In some embodiments, a target cell is isolated from a subject having β-thalassemia.

### Stem Cells

Methods of the disclosure can be used with stem cells. Stem cells are typically cells that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs). Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

Pluripotent stem cells are generally known in the art. The present disclosure provides technologies (e.g., systems, compositions, methods, etc.) related to pluripotent stem cells. In some embodiments, pluripotent stem cells are stem cells that: (a) are capable of inducing teratomas when transplanted in immunodeficient (SCID) mice; (b) are capable of differentiating to cell types of all three germ layers (e.g., can differentiate to ectodermal, mesodermal, and endodermal cell types); and/or (c) express one or more markers of embryonic stem cells (e.g., human embryonic stem cells express Oct-4, alkaline phosphatase, SSEA-3 surface antigen, SSEA-4 surface antigen, nanog, TRA-1-60, TRA-1-81, Sox-2, REX1, etc.). In some aspects, human pluripotent stem cells do not show expression of differentiation markers. In some embodiments, ES cells and/or iPSCs edited using methods of the disclosure maintain their pluripotency, e.g., (a) are capable of inducing teratomas when transplanted in immunodeficient (SCID) mice; (b) are capable of differentiating to cell types of all three germ layers, e.g., can differentiate to ectodermal, mesodermal, and endodermal cell types); and/or (c) express one or more markers of embryonic stem cells.

In some embodiments, ES cells (e.g., human ES cells) can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis, or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

### iPS Cells

Induced pluripotent stem cells (iPSC) are a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such as Oct-3/4 (Pouf51) and Sox-2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct-3/4, Sox-2, Klf4, and/or c-Myc using a retroviral system or with Oct-4, Sox-2, NANOG, and/or LIN28 using a lentiviral system. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al., Science 2007; 318(5854): 1224 or Takahashi et al., Cell 2007; 131:861-72. Numerous suitable methods for reprogramming are known to those of skill in the art, and the present disclosure is not limited in this respect.

In some embodiments, a target cell for the editing and cargo integration methods described herein is an iPSC, wherein the edited iPSC is then differentiated, e.g., into an iPSC-derived immune cell. In some embodiments, the differentiated cell is an iPSC-derived immune cell. In some embodiments, the differentiated cell is an iPSC-derived iNK cell, an iPSC-derived T cell (e.g., an iPSC-derived alpha-beta T cell, gamma-delta T cell, Treg, CD4+ T cell, or CD8+ T cell), an iPSC-derived dendritic cell, or an iPSC-derived macrophage. In some embodiments, the differentiated cell is an iPSC-derived pancreatic beta cell.

### iNK Cells

In some embodiments, the present disclosure provides methods of generating iNK cells (e.g., genetically modified iNK cells), e.g., derived from a genetically modified stem cell (e.g., iPSC).

In some embodiments, genetic modifications present in an iNK cell of the present disclosure can be made at any stage during the reprogramming process from donor cell to iPSC, during the iPSC stage, and/or at any stage of the process of differentiating the iPSC to an iNK state, e.g., at an intermediary state, such as, for example, an iPSC-derived HSC state, or even up to or at the final iNK cell state.

For example, one or more genomic modifications present in a genetically modified iNK cell of the present disclosure may be made at one or more different cell stages (e.g., reprogramming from donor to iPSC, differentiation of iPSC to iNK). In some embodiments, one or more genomic modifications present in a genetically modified iNK cell provided herein is made before reprogramming a donor cell to an iPSC state. In some embodiments, all edits present in a genetically modified iNK cell provided herein are made at the same time, in close temporal proximity, and/or at the same cell stage of the reprogramming/differentiation process, e.g., at the donor cell stage, during the reprogramming process, at the iPSC stage, or during the differentiation process, e.g., from iPSC to iNK. In some embodiments, two or more edits present in a genetically modified iNK cell provided herein are made at different times and/or at different cell stages of the reprogramming/differentiation process from donor cell to iPSC to iNK. For example, in some embodiments, a first edit is made at the donor cell stage and a second (different) edit is made at the iPSC stage. In some embodiments, a first edit is made at the reprogramming stage (e.g., donor to iPSC) and a second (different) edit is made at the iPSC stage.

A variety of cell types can be used as a donor cell that can be subjected to reprogramming, differentiation, and/or genetic engineering strategies described herein. For example, the donor cell can be a pluripotent stem cell or a differentiated cell, e.g., a somatic cell, such as, for example, a fibroblast or a T lymphocyte. In some embodiments, donor cells are manipulated (e.g., subjected to reprogramming, differentiation, and/or genetic engineering) to generate iNK cells described herein.

A donor cell can be from any suitable organism. For example, in some embodiments, the donor cell is a mammalian cell, e.g., a human cell or a non-human primate cell. In some embodiments, the donor cell is a somatic cell. In some embodiments, the donor cell is a stem cell or progenitor cell. In certain embodiments, the donor cell is not or was not part of a human embryo and its derivation does not involve destruction of a human embryo.

In some embodiments, a genetically modified iNK cell is derived from an iPSC, which in turn is derived from a somatic donor cell. Any suitable somatic cell can be used in the generation of iPSCs, and in turn, the generation of iNK cells. Suitable strategies for deriving iPSCs from various somatic donor cell types have been described and are known in the art. In some embodiments, a somatic donor cell is a fibroblast cell. In some embodiments, a somatic donor cell is a mature T cell.

For example, in some embodiments, a somatic donor cell, from which an iPSC, and subsequently an iNK cell is derived, is a developmentally mature T cell (a T cell that has undergone thymic selection). One hallmark of developmentally mature T cells is a rearranged T cell receptor locus. During T cell maturation, the TCR locus undergoes V(D)J rearrangements to generate complete V-domain exons. These rearrangements are retained throughout reprogramming of a T cells to an iPSC, and throughout differentiation of the resulting iPSC to a somatic cell.

In certain embodiments, a somatic donor cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, or a CD4⁺ CD25⁺ CD127⁻Foxp3⁺ T cell.

T cells can be advantageous for the generation of iPSCs. For example, T cells can be edited with relative ease, e.g., by CRISPR-based methods or other genetic engineering methods. Additionally, the rearranged TCR locus allows for genetic tracking of individual cells and their daughter cells. For example, if the reprogramming, expansion, culture, and/or differentiation strategies involved in the generation of NK cells a clonal expansion of a single cell, the rearranged TCR locus can be used as a genetic marker unambiguously identifying a cell and its daughter cells. This, in turn, allows for the characterization of a cell population as truly clonal, or for the identification of mixed populations, or contaminating cells in a clonal population. Another potential advantage of using T cells in generating iNK cells carrying multiple edits is that certain karyotypic aberrations associated with chromosomal translocations are selected against in T cell culture. Such aberrations can pose a concern when editing cells by CRISPR technology, and in particular when generating cells carrying multiple edits. Using T cell derived iPSCs as a starting point for the derivation of therapeutic lymphocytes can allow for the expression of a pre-screened TCR in the lymphocytes, e.g., via selecting the T cells for binding activity against a specific antigen, e.g., a tumor antigen, reprogramming the selected T cells to iPSCs, and then deriving lymphocytes from these iPSCs that express the TCR (e.g., T cells). This strategy can allow for activating the TCR in other cell types, e.g., by genetic or epigenetic strategies. Additionally, T cells retain at least part of their “epigenetic memory” throughout the reprogramming process, and thus subsequent differentiation of the same or a closely related cell type, such as iNK cells can be more efficient and/or result in higher quality cell populations as compared to approaches using non-related cells, such as fibroblasts, as a starting point for iNK derivation.

In some embodiments, a donor cell being manipulated, e.g., a cell being reprogrammed and/or undergoing genetic engineering as described herein, is one or more of a long-term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a fibroblast, a monocyte-derived macrophage or dendritic cell, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte.

In some embodiments, a donor cell is one or more of a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In some embodiments, a donor cell is one or more of a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In some embodiments, a donor cell is one or more of a myeloid progenitor cell (e.g., a common myeloid progenitor (CMP) cell or granulocyte macrophage progenitor (GMP) cell). In some embodiments, a donor cell is one or more of a lymphoid progenitor cell, e.g., a common lymphoid progenitor (CLP) cell. In some embodiments, a donor cell is one or more of an erythroid progenitor cell (e.g., an MEP cell). In some embodiments, a donor cell is one or more of a hematopoietic stem/progenitor cell (e.g., a long term HSC (LT-HSC), short term HSC (ST-HSC), MPP cell, or lineage restricted progenitor (LRP) cell). In certain embodiments, the donor cell is a CD34⁺ cell, CD34⁺CD90⁺ cell, CD34⁺CD38⁻ cell, CD34⁺CD90⁺CD49f⁺CD38⁺CD45RA; cell, CD105⁺ cell, CD31⁺, or CD133⁺ cell, or a CD34⁺CD90⁺ CD133⁺ cell. In some embodiments, a donor cell is one or more of an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In some embodiments, a donor cell is one or more of a mobilized peripheral blood hematopoietic CD34⁺ cell (after the subject is treated with a mobilization agent, e.g., G-CSF or Plerixafor). In some embodiments, a donor cell is a peripheral blood endothelial cell. In some embodiments, a donor cell is a peripheral blood natural killer cell.

In some embodiments, a donor cell is a dividing cell. In some embodiments, a donor cell is a non-dividing cell.

In some embodiments, a genetically modified (e.g., edited) iNK cell resulting from one or more methods and/or strategies described herein, are administered to a subject in need thereof, e.g., in the context of an immuno-oncology therapeutic approach. In some embodiments, donor cells, or any cells of any stage of the reprogramming, differentiating, and/or genetic engineering strategies provided herein, can be maintained in culture or stored (e.g., frozen in liquid nitrogen) using any suitable method known in the art, e.g., for subsequent characterization or administration to a subject in need thereof.

### Genetically Modified Cells

### Loss-of-Function Modifications

In some embodiments, a target cell described herein (e.g., an NK cell or a stem cell (e.g., iPSC) described herein) is genetically engineered to introduce a disruption (e.g., a knockout) in one or more targets described herein. For example, in some embodiments, a target cell described herein (e.g., an NK cell or a stem cell (e.g., iPSC) described herein) can be genetically engineered to knockout all or a portion of one or more target gene, introduce a frameshift in one or more target genes, and/or cause a truncation of an encoded gene product (e.g., by introducing a premature stop codon). In some embodiments, a target cell described herein (e.g., an NK cell or a stem cell (e.g., iPSC) described herein) can be genetically engineered to knockout all or a portion of a target gene using a gene-editing system, e.g., as described herein. In some such embodiments, a gene-editing system may be or comprise a CRISPR system, a zinc finger nuclease system, a TALEN, and/or a meganuclease.

In some embodiments, the present disclosure provides methods suitable for high-efficiency knockout (e.g., a high proportion of a cell population comprises a knockout). In some embodiments, high-efficiency knockout results in at least 65% of the cells in a population of cells comprising a knockout (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells comprise a knockout).

In certain embodiments, the disclosure provides a genetically engineered target cell described herein (e.g., an NK cell or a stem cell (e.g., iPSC) described herein), and/or progeny cell, comprising a disruption in TGF signaling, e.g., TGF beta signaling. In some embodiments, this is useful, for example, in circumstances where it is desirable to generate a differentiated cell (e.g., an NK cell) from pluripotent stem cell, wherein TGF signaling, e.g., TGF beta signaling is disrupted in the differentiated cell.

TGF beta signaling inhibits or decreases the survival and/or activity of some differentiated cell types that are useful for therapeutic applications, e.g., TGF beta signaling is a negative regulator of natural killer cells, which can be used in immunotherapeutic applications. In some embodiments, it is desirable to generate a clinically effective number of natural killer cells comprising a genetic modification that disrupts TGF beta signaling, thus avoiding the negative effect of TGF beta on the clinical effectiveness of such cells. It is advantageous, in some embodiments, to source such NK cells from a pluripotent stem cell, instead, for example, from mature NK cells obtained from a donor. Modifying a stem cell instead of a differentiated cell has, among others, the advantage of allowing for clonal derivation, characterization, and/or expansion of a specific genotype, e.g., a specific stem cell clone harboring a specific genetic modification (e.g., a targeted disruption of TGFβRII in the absence of any undesired (e.g., off-target) modifications). In some embodiments, a stem cell, e.g., a human iPSC, is genetically engineered not to express one or more TGFβ receptor, e.g., TGFβRII, or to express a dominant negative variant of a TGFβ receptor, e.g., a dominant negative TGFβRII variant. Exemplary sequences of TGFβRII are set forth in KR710923.1, NM_001024847.2, and NM_003242.5. An exemplary dominant negative TGFβRII is disclosed in Immunity. 2000 February; 12(2):171-81.

In certain embodiments, the disclosure provides a genetically engineered target cell described herein (e.g., an NK cell or a stem cell (e.g., iPSC) described herein), and/or progeny cell, that additionally or alternatively comprises a disruption in interleukin signaling, e.g., IL-15 signaling. IL-15 is a cytokine with structural similarity to Interleukin-2 (IL-2), which binds to and signals through a complex composed of IL-2/IL-15 receptor beta chain (CD122) and the common gamma chain (gamma-C, CD132). Exemplary sequences of IL-15 are provided in NG_029605.2. Disruption of IL-15 signaling may be useful, for example, in circumstances where it is desirable to generate a differentiated cell from a pluripotent stem cell, but with certain signaling pathways (e.g., IL-15) disrupted in the differentiated cell. IL-15 signaling can inhibit or decrease survival and/or activity of some types of differentiated cells, such as cells that may be useful for therapeutic applications. For example, IL-15 signaling is a negative regulator of natural killer (NK) cells.

CISH (encoded by the CISH gene) is downstream of the IL-15 receptor and can act as a negative regulator of IL-15 signaling in NK cells. As used herein, the term “CISH” refers to the Cytokine Inducible SH2 Containing Protein (see, e.g., Delconte et al., Nat Immunol. 2016 July; 17(7):816-24; exemplary sequences for CISH are set forth as NG_023194.1). In some embodiments, disruption of CISH regulation may increase activation of Jak/STAT pathways, leading to increased survival, proliferation and/or effector functions of NK cells. Thus, in some embodiments, genetically engineered NK cells (e.g., iNK cells, e.g., generated from genetically engineered hiPSCs comprising a disruption of CISH regulation) exhibit greater responsiveness to IL-15-mediated signaling than non-genetically engineered NK cells. In some such embodiments, genetically engineered NK cells exhibit greater effector function relative to non-genetically engineered NK cells.

In some embodiments, a genetically engineered NK cell, stem cell and/or progeny cell, additionally or alternatively, comprises a disruption and/or loss of function in one or more of B2M, NKG2A, PD1, TIGIT, ADORA2a, CIITA, HLA class II histocompatibility antigen alpha chain genes, HLA class II histocompatibility antigen beta chain genes, CD32B, or TRAC.

As used herein, the term “B2M” (β2 microglobulin) refers to a serum protein found in association with the major histocompatibility complex (MHC) class I heavy chain on the surface of nearly all nucleated cells. Exemplary sequences for B2M are set forth as NG 012920.2.

As used herein, the term “NKG2A” (natural killer group 2A) refers to a protein belonging to the killer cell lectin-like receptor family, also called NKG2 family, which is a group of transmembrane proteins preferentially expressed in NK cells. This family of proteins is characterized by the type II membrane orientation and the presence of a C-type lectin domain. See, e.g., Kamiya-T et al., J Clin Invest 2019 https://doi.org/10.1172/JCI123955. Exemplary sequences for NKG2A are set forth as AF461812.1.

As used herein, the term “PD1” (Programmed cell death protein 1), also known CD279 (cluster of differentiation 279), refers to a protein found on the surface of cells that has a role in regulating the immune system's response to the cells of the human body by down-regulating the immune system and promoting self-tolerance by suppressing T cell inflammatory activity. PD1 is an immune checkpoint and guards against autoimmunity. Exemplary sequences for PD1 are set forth as NM_005018.3.

As used herein, the term “TIGIT” (T cell immunoreceptor with Ig and ITIM domains) refers to a member of the PVR (poliovirus receptor) family of immunoglobulin proteins. The product of this gene is expressed on several classes of T cells including follicular B helper T cells (TFH). Exemplary sequences for TIGIT are set forth in NM 173799.4.

As used herein, the term “ADORA2A” refers to the adenosine A2a receptor, a member of the guanine nucleotide-binding protein (G protein)-coupled receptor (GPCR) superfamily, which is subdivided into classes and subtypes. This protein, an adenosine receptor of A2A subtype, uses adenosine as the preferred endogenous agonist and preferentially interacts with the G(s) and G(olf) family of G proteins to increase intracellular cAMP levels. Exemplary sequences of ADORA2a are provided in NG_052804.1.

As used herein, the term “CIITA” refers to the protein located in the nucleus that acts as a positive regulator of class II major histocompatibility complex gene transcription, and is referred to as the “master control factor” for the expression of these genes. The protein also binds GTP and uses GTP binding to facilitate its own transport into the nucleus. Mutations in this gene have been associated with bare lymphocyte syndrome type II (also known as hereditary MHC class II deficiency or HLA class II-deficient combined immunodeficiency), increased susceptibility to rheumatoid arthritis, multiple sclerosis, and possibly myocardial infarction. See, e.g., Chang et al., J Exp Med 180: 1367-1374; and Chang et al., Immunity. 1996 February; 4(2): 167-78, the entire contents of each of which are incorporated by reference herein. An exemplary sequence of CIITA is set forth as NG_009628.1.

In some embodiments, two or more HLA class II histocompatibility antigen alpha chain genes and/or two or more HLA class II histocompatibility antigen beta chain genes are disrupted, e.g., knocked out, e.g., by genomic editing. For example, in some embodiments, two or more HLA class II histocompatibility antigen alpha chain genes selected from HLA-DQA1, HLA-DRA, HLA-DPA1, HLA-DMA, HLA-DQA2, and HLA-DOA are disrupted, e.g., knocked out. For another example, in some embodiments, two or more HLA class II histocompatibility antigen beta chain genes selected from HLA-DMB, HLA-DOB, HLA-DPB1, HLA-DQB1, HLA-DQB3, HLA-DQB2, HLA-DRB1, HLA-DRB3, HLA-DRB4, and HLA-DRB5 are disrupted, e.g., knocked out. See, e.g., Crivello et al., J Immunol January 2019, ji1800257; DOI: https://doi.org/10.4049/jimmunol.1800257, the entire contents of which are incorporated herein by reference.

As used herein, the term “CD32B” (cluster of differentiation 32B) refers to a low affinity immunoglobulin gamma Fc region receptor II-b protein that, in humans, is encoded by the FCGR2B gene. See, e.g., Rankin-CT et al., Blood 2006 108(7):2384-91, the entire contents of which are incorporated herein by reference.

As used herein, the term “TRAC” refers to the T-cell receptor alpha subunit (constant), encoded by the TRAC locus.

### Gain-of-Function Modifications

In some embodiments, a target cell described herein (e.g., an NK cell or a stem cell (e.g., iPSC) described herein) can additionally be genetically engineered to comprise a genetic modification that leads to expression of one or more gene products of interest described herein using, e.g., a gene-editing system, e.g., as described herein. In some such embodiments, a gene-editing system may be or comprise a CRISPR system, a zinc finger nuclease system, a TALEN, and/or a meganuclease.

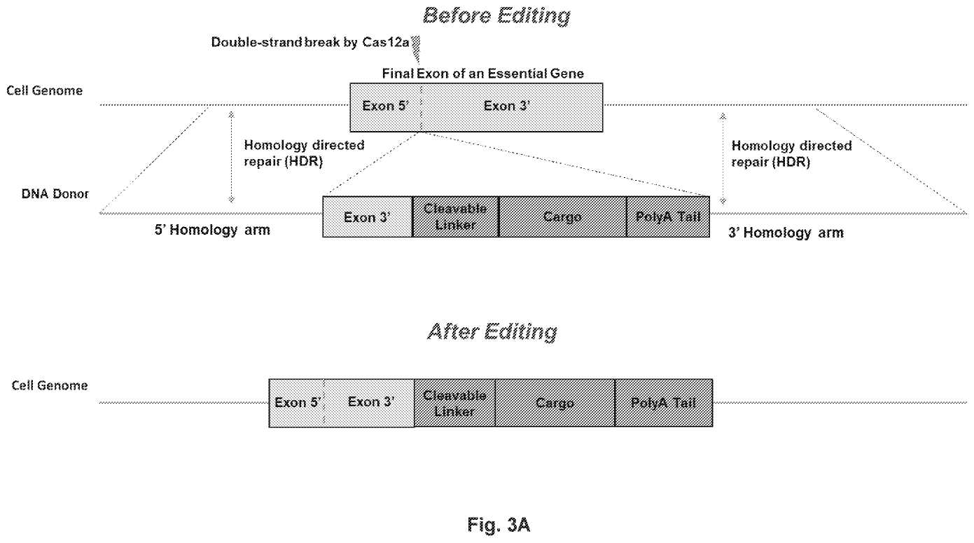

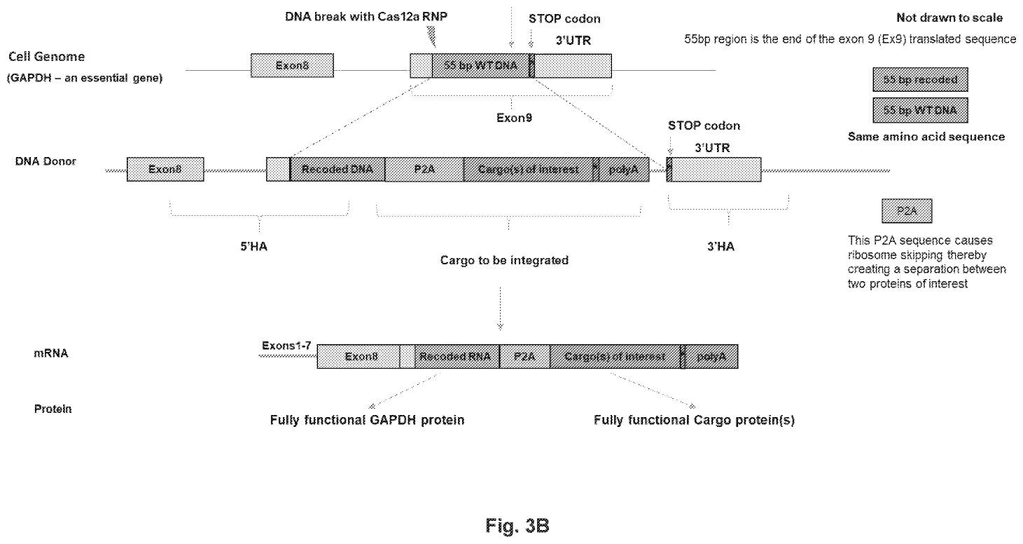

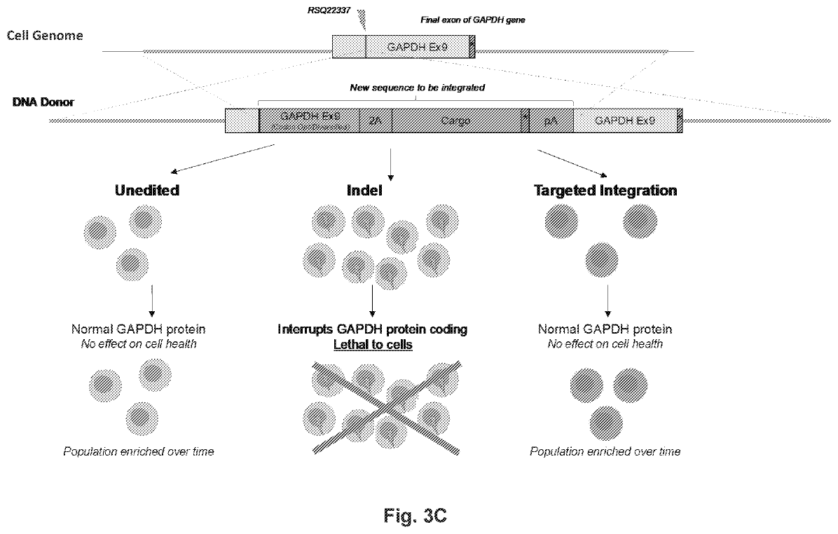

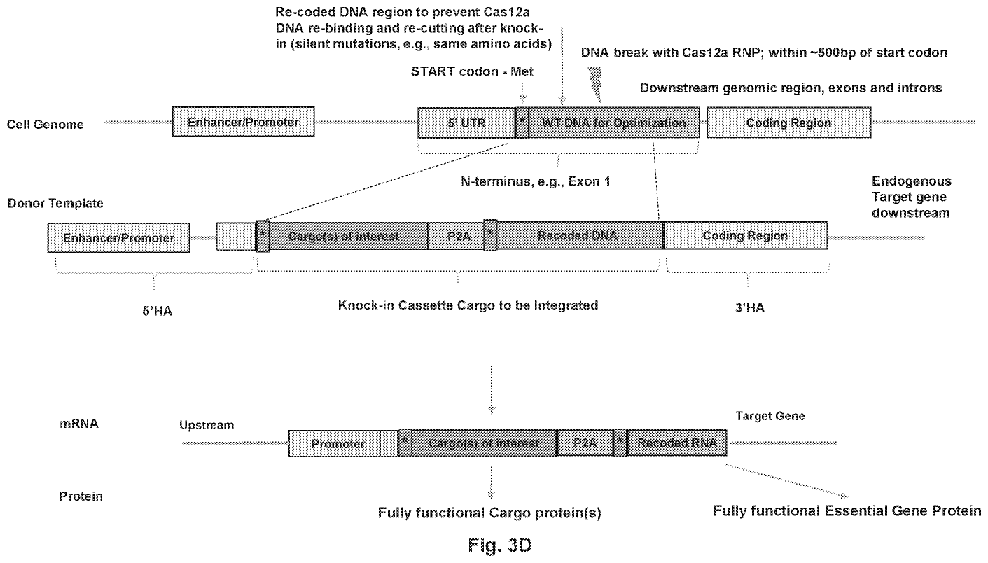

In some embodiments, a cell is produced by a method of the present disclosure, e.g., a method that comprises contacting the cell with a nuclease that causes a break within an endogenous coding sequence of an essential gene in the cell wherein the essential gene encodes at least one gene product that is required for survival and/or proliferation of the cell. The cell is also contacted with a donor template that comprises a knock-in cassette comprising an exogenous coding sequence for a gene product of interest in frame with and downstream (3′) of an exogenous coding sequence or partial coding sequence of the essential gene. The knock-in cassette is integrated into the genome of the cell by homology-directed repair (HDR) of the break, resulting in a genome-edited cell that expresses the gene product of interest and the gene product encoded by the essential gene that is required for survival and/or proliferation of the cell, or a functional variant thereof. This is illustrated in FIG. 3 for an exemplary method. In some embodiments, a cell is contacted with a donor template that comprises a knock-in cassette comprising an exogenous coding sequence for a gene product of interest in frame with and upstream (5′) of an exogenous coding sequence or partial coding sequence of the essential gene.

In some embodiments, the cell comprises a genome with an exogenous coding sequence for a gene product of interest in frame with and downstream (3′) of a coding sequence of an essential gene, wherein the essential gene encodes a gene product that is required for survival and/or proliferation of the cell.

In some embodiments, the cell comprises a genome with an exogenous coding sequence for a gene product of interest in frame with and upstream (5′) of a coding sequence of an essential gene, wherein the essential gene encodes a gene product that is required for survival and/or proliferation of the cell.

In some embodiments, the cell comprises a genomic modification, wherein the genomic modification comprises an insertion of an exogenous knock-in cassette within an endogenous coding sequence of an essential gene in the cell's genome, wherein the essential gene encodes a gene product that is required for survival and/or proliferation of the cell, wherein the knock-in cassette comprises an exogenous coding sequence for a gene product of interest in frame with and downstream (3′) of an exogenous coding sequence or partial coding sequence encoding the gene product of the essential gene, or a functional variant thereof, and wherein the cell expresses the gene product of interest and the gene product encoded by the essential gene that is required for survival and/or proliferation of the cell, or a functional variant thereof. In some embodiments, the gene product of interest and the gene product encoded by the essential gene are expressed from the endogenous promoter of the essential gene.

### Donor Template

In one aspect the present disclosure provides a donor template comprising a knock-in cassette with an exogenous coding sequence for a gene product of interest in frame with and downstream (3′) of an exogenous coding sequence or partial coding sequence of an essential gene, wherein the essential gene encodes a gene product that is required for survival and/or proliferation of the cell.

In one aspect the present disclosure provides an impetus for designing donor templates comprising a knock-in cassette with an exogenous coding sequence for a gene product of interest in frame with and upstream (5′) of an exogenous coding sequence or partial coding sequence of an essential gene, wherein the essential gene encodes a gene product that is required for survival and/or proliferation of the cell; see e.g., FIG. 3D.

In some embodiments, the donor template is for use in editing the genome of a cell by homology-directed repair (HDR).

Donor template design is described in detail in the literature, for instance in PCT Publication No. WO2016/073990A1. Donor templates can be single-stranded or double-stranded and can be used to facilitate HDR-based repair of double-strand breaks (DSBs), and are particularly useful for inserting a new sequence into the target sequence, or replacing the target sequence altogether. In some embodiments, the donor template is a donor DNA template. In some embodiments the donor DNA template is double-stranded.

Whether single-stranded or double stranded, donor templates generally include regions that are homologous to regions of DNA within or near (e.g., flanking or adjoining) a target sequence to be cleaved. These homologous regions are referred to herein as “homology arms,” and are illustrated schematically below relative to the knock-in cassette (which may be separated from one or both of the homology arms by additional spacer sequences that are not shown):
[5′ homology arm]-[knock-in cassette]-[3′ homology arm].

The homology arms can have any suitable length (including 0 nucleotides if only one homology arm is used), and 5′ and 3′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements.

A donor template can be a nucleic acid vector, such as a viral genome or circular double-stranded DNA, e.g., a plasmid. Nucleic acid vectors comprising donor templates can include other coding or non-coding elements. For example, a donor template nucleic acid can be delivered as part of a viral genome (e.g., in an AAV, adenoviral, Sendai virus, or lentiviral genome) that includes certain genomic backbone elements (e.g., inverted terminal repeats, in the case of an AAV genome). In some embodiments, a donor template is comprised in a plasmid that has not been linearized. In some embodiments, a donor template is comprised in a plasmid that has been linearized. In some embodiments, a donor template is comprised within a linear dsDNA fragment. In some embodiments, a donor template nucleic acid can be delivered as part of an AAV genome. In some embodiments, a donor template nucleic acid can be delivered as a single stranded oligo donor (ssODN), for example, as a long multi-kb ssODN derived from m13 phage synthesis, or alternatively, short ssODNs, e.g., that comprise small genes of interest, tags, and/or probes. In some embodiments, a donor template nucleic acid can be delivered as a Doggybone™ DNA (dbDNA™) template. In some embodiments, a donor template nucleic acid can be delivered as a DNA minicircle. In some embodiments, a donor template nucleic acid can be delivered as an Integration-deficient Lentiviral Particle (IDLV). In some embodiments, a donor template nucleic acid can be delivered as a MMLV-derived retrovirus. In some embodiments, a donor template nucleic acid can be delivered as a piggyBac™ sequence. In some embodiments, a donor template nucleic acid can be delivered as a replicating EBNA1 episome.

In certain embodiments, the 5′ homology arm may be about 25 to about 1,000 base pairs in length, e.g., at least about 100, 200, 400, 600, or 800 base pairs in length. In certain embodiments, the 5′ homology arm comprises about 50 to 800 base pairs, e.g., 100 to 800, 200 to 800, 400 to 800, 400 to 600, or 600 to 800 base pairs. In certain embodiments, the 3′ homology arm may be about 25 to about 1,000 base pairs in length, e.g., at least about 100, 200, 400, 600, or 800 base pairs in length. In certain embodiments, the 3′ homology arm comprises about 50 to 800 base pairs, e.g., 100 to 800, 200 to 800, 400 to 800, 400 to 600, or 600 to 800 base pairs. In certain embodiments, the 5′ and 3′ homology arms are symmetrical in length. In certain embodiments, the 5′ and 3′ homology arms are asymmetrical in length.

In certain embodiments, a 5′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than about 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 5′ homology arm is less than about 1,000 base pairs, less than about 900 base pairs, less than about 800 base pairs, is less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 5′ homology arm is about 400-600 base pairs, e.g., about 500 base pairs.

In certain embodiments, a 3′ homology arm is less than about 3,000 base pairs, less than about 2,900 base pairs, less than about 2,800 base pairs, less than about 2,700 base pairs, less than about 2,600 base pairs, less than about 2,500 base pairs, less than about 2,400 base pairs, less than about 2,300 base pairs, less than about 2,200 base pairs, less than about 2,100 base pairs, less than about 2,000 base pairs, less than about 1,900 base pairs, less than about 1,800 base pairs, less than about 1,700 base pairs, less than about 1,600 base pairs, less than about 1,500 base pairs, less than about 1,400 base pairs, less than about 1,300 base pairs, less than about 1,200 base pairs, less than about 1,100 base pairs, less than 1,000 base pairs, less than about 900 base pairs, less than about 800 base pairs, less than about 700 base pairs, less than about 600 base pairs, less than about 500 base pairs, or less than about 400 base pairs.

In certain embodiments, e.g., where a viral vector is utilized to introduce a knock-in cassette through a method described herein, a 3′ homology arm is less than about 1,000 base pairs, less than about 900 base pairs, less than about 800 base pairs, less than about 700 base pairs, less than about 600 base pairs, less than about 500 base pairs, less than about 400 base pairs, or less than about 300 base pairs. In certain embodiments, e.g., where a viral vector is utilized to introduce a knock-in cassette through a method described herein, a 3′ homology arm is about 400-600 base pairs, e.g., about 500 base pairs.

In certain embodiments, the 5′ and 3′ homology arms flank the break and are less than 100, 75, 50, 25, 15, 10 or 5 base pairs away from an edge of the break. In certain embodiments, the 5′ and 3′ homology arms flank an endogenous stop codon. In certain embodiments, the 5′ and 3′ homology arms flank a break located within about 500 base pairs (e.g., about 500 base pairs, about 450 base pairs, about 400 base pairs, about 350 base pairs, about 300 base pairs, about 250 base pairs, about 200 base pairs, about 150 base pairs, about 100 base pairs, about 50 base pairs, or about 25 base pairs) upstream (5′) of an endogenous stop codon, e.g., the stop codon of an essential gene. In certain embodiments, the 5′ homology arm encompasses an edge of the break.

### Knock-In Cassette

In some embodiments, the knock-in cassette within the donor template comprises an exogenous coding sequence for the gene product of interest in frame with and downstream (3′) of an exogenous coding sequence or partial coding sequence of the essential gene. In some embodiments, a knock-in cassette within a donor template comprises an exogenous coding sequence for the gene product of interest in frame with and upstream (5′) of an exogenous coding sequence or partial coding sequence of an essential gene. In some embodiments, the knock-in cassette is a polycistronic knock-in cassette. In some embodiments, the knock-in cassette is a bicistronic knock-in cassette. In some embodiment the knock-in cassette does not comprise a reporter gene, e.g., a fluorescent reporter gene or an antibiotic resistance gene.

In some embodiments, a single essential gene locus will be targeted by two knock-in cassettes comprising different “cargo” sequences. In some embodiments, one allele will incorporate one knock-in cassette, while the other allele will incorporate the other knock-in cassette. In some embodiments, a gRNA utilized to generate an appropriate DNA break may be the same for each of the two different knock-in cassettes. In some embodiments, gRNAs utilized to generate appropriate DNA breaks for each of the two different knock-in cassettes may be different, such that the “cargo” sequence is incorporated at a different position for each allele. In some embodiments, such a different position for each allele may still be within the ultimate exons coding region. In some embodiments, such a different position for each allele may be within the penultimate exon (second to last), and/or ultimate (last) exons coding region. In some embodiments, such a different position for at least one of the alleles may be within the first exon. In some embodiments, such a different position for at least one of the alleles may be within the first or second exon.

In order to properly restore the essential gene coding region in the genetically modified cell (so that a functioning gene product is produced) the knock-in cassette does not need to comprise an exogenous coding sequence that corresponds to the entire coding sequence of the essential gene. Indeed, depending on the location of the break in the endogenous coding sequence of the essential gene it may be possible to restore the essential gene by providing a knock-in cassette that comprises a partial coding sequence of the essential gene, e.g., that corresponds to a portion of the endogenous coding sequence of the essential gene that spans the break and the entire region downstream of the break (minus the stop codon), and/or that corresponds to a portion of the endogenous coding sequence of the essential gene that spans the break and the entire region upstream of the break (up to and optionally including the start codon).

In order to minimize the size of the knock-in cassette it may in fact be advantageous, in some embodiments, to have the break located within the last 1500, 1000, 750, 500, 400, 300, 200, 100, or 50 base pairs of the endogenous coding sequence of the essential gene, i.e., towards the 3′ end of the coding sequence. In some embodiments, a base pair's location in a coding sequence may be defined 3′-to-5′ from an endogenous translational stop signal (e.g., a stop codon). In some embodiments, as used herein, an “endogenous coding sequence” can include both exonic and intronic base pairs, and refers to gene sequence occurring 5′ to an endogenous functional translational stop signal. In some embodiments, a break within an endogenous coding sequence comprises a break within one DNA strand. In some embodiments, a break within an endogenous coding sequence comprises a break within both DNA strands. In some embodiments, a break is located within the last 1000 base pairs of the endogenous coding sequence. In some embodiments, a break is located within the last 750 base pairs of the endogenous coding sequence. In some embodiments, a break is located within the last 600 base pairs of the endogenous coding sequence. In some embodiments, a break is located within the last 500 base pairs of the endogenous coding sequence. In some embodiments, a break is located within the last 400 base pairs of the endogenous coding sequence. In some embodiments, a break is located within the last 300 base pairs of the endogenous coding sequence. In some embodiments, a break is located within the last 250 base pairs of the endogenous coding sequence. In some embodiments, a break is located within the last 200 base pairs of the endogenous coding sequence. In some embodiments, a break is located within the last 150 base pairs of the endogenous coding sequence. In some embodiments, a break is located within the last 100 base pairs of the endogenous coding sequence. In some embodiments, a break is located within the last 75 base pairs of the endogenous coding sequence. In some embodiments, a break is located within the last 50 base pairs of the endogenous coding sequence. In some embodiments, a break is located within the last 21 base pairs of the endogenous coding sequence.

In some embodiments, the exogenous partial coding sequence of the essential gene in the knock-in cassette encodes a C-terminal fragment of a protein encoded by the essential gene, e.g., a fragment that is less than 500, 250, 150, 125, 100, 75, 50, 25, 20, 15 or 10 amino acids in length. In some embodiments, the exogenous partial coding sequence of the essential gene in the knock-in cassette is codon optimized. In some embodiments, the exogenous partial coding sequence of the essential gene in the knock-in cassette is codon optimized to eliminate at least one PAM site. In some embodiments, the exogenous partial coding sequence of the essential gene in the knock-in cassette is codon optimized to eliminate more than one PAM site. In some embodiments, the exogenous partial coding sequence of the essential gene in the knock-in cassette is codon optimized to eliminate all relevant nuclease specific PAM sites. In some embodiments, a C-terminal fragment of a protein encoded by the essential gene is about 140 amino acids in length. In some embodiments, a C-terminal fragment of a protein encoded by the essential gene is about 130 amino acids in length. In some embodiments, a C-terminal fragment of a protein encoded by the essential gene is about 120 amino acids in length. In some embodiments, the C-terminal fragment includes an amino acid sequence that is encoded by a region of the endogenous coding sequence of the essential gene that spans the break. In some embodiments, a C-terminal fragment includes an amino acid sequence that is encoded by a region of the endogenous coding sequence within 1 exon of the essential gene. In some embodiments, a C-terminal fragment includes an amino acid sequence that is encoded by a region of the endogenous coding sequence within 2 exons of the essential gene. In some embodiments, a C-terminal fragment includes an amino acid sequence that is encoded by a region of the endogenous coding sequence within 3 exons of the essential gene. In some embodiments, a C-terminal fragment includes an amino acid sequence that is encoded by a region of the endogenous coding sequence within 4 exons of the essential gene. In some embodiments, a C-terminal fragment includes an amino acid sequence that is encoded by a region of the endogenous coding sequence within 5 exons of the essential gene.

In some embodiments, the exogenous partial coding sequence of an essential gene in a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid C-terminal fragment of a protein encoded by an essential gene. In some embodiments, the exogenous partial coding sequence of an essential gene in a knock-in cassette encodes a 19 amino acid C-terminal fragment of a protein encoded by an essential gene. In some embodiments, the exogenous partial coding sequence of an essential gene in a knock-in cassette encodes an 18 amino acid C-terminal fragment of a protein encoded by an essential gene. In some embodiments, the exogenous partial coding sequence of an essential gene in a knock-in cassette encodes a 17 amino acid C-terminal fragment of a protein encoded by an essential gene. In some embodiments, the exogenous partial coding sequence of an essential gene in a knock-in cassette encodes a 16 amino acid C-terminal fragment of a protein encoded by an essential gene. In some embodiments, the exogenous partial coding sequence of an essential gene in a knock-in cassette encodes a 1 amino acid C-terminal fragment of a protein encoded by an essential gene.

In some embodiments, e.g., when the essential gene includes many exons as shown in the exemplary method of FIG. 3A, it may be advantageous to have the break within the last exon of the essential gene. In some embodiments, e.g., when the essential gene includes many exons as shown in the exemplary method of FIG. 3A, it may be advantageous to have the break within the penultimate exon of the essential gene. It is to be understood however that the present disclosure is not limited to any particular location for the break and that the available positions will vary depending on the nature and length of the essential gene and the length of the exogenous coding sequence for the gene product of interest. For example, for essential genes that include a few exons or when the gene product of interest is small it may be possible to locate the break in an upstream exon.

In order to minimize the size of the knock-in cassette it may in fact be advantageous, in some embodiments, to have the break located within the first 1500, 1000, 750, 500, 400, 300, 200, 100, or 50 base pairs of an endogenous coding sequence of the essential gene, i.e., starting from the 5′ end of a coding sequence. In some embodiments, a base pair's location in a coding sequence may be defined 5′-to-3′ from an endogenous translational start signal (e.g., a start codon). In some embodiments, as used herein, an “endogenous coding sequence” can include both exonic and intronic base pairs, and refers to gene sequence occurring 3′ to an endogenous functional translational start signal. In some embodiments, a break within an endogenous coding sequence comprises a break within one DNA strand. In some embodiments, a break within an endogenous coding sequence comprises a break within both DNA strands. In some embodiments, a break is located within the first 1000 base pairs of the endogenous coding sequence. In some embodiments, a break is located within the first 750 base pairs of an endogenous coding sequence. In some embodiments, a break is located within the first 600 base pairs of the endogenous coding sequence. In some embodiments, a break is located within the first 500 base pairs of the endogenous coding sequence. In some embodiments, a break is located within the first 400 base pairs of the endogenous coding sequence. In some embodiments, a break is located within the first 300 base pairs of the endogenous coding sequence. In some embodiments, a break is located within the first 250 base pairs of the endogenous coding sequence. In some embodiments, a break is located within the first 200 base pairs of the endogenous coding sequence. In some embodiments, a break is located within the first 150 base pairs of the endogenous coding sequence. In some embodiments, a break is located within the first 100 base pairs of the endogenous coding sequence. In some embodiments, a break is located within the first 75 base pairs of the endogenous coding sequence. In some embodiments, a break is located within the first 50 base pairs of the endogenous coding sequence. In some embodiments, a break is located within the first 21 base pairs of the endogenous coding sequence.

In some embodiments, the exogenous partial coding sequence of the essential gene in the knock-in cassette encodes an N-terminal fragment of a protein encoded by the essential gene, e.g., a fragment that is less than 500, 250, 150, 125, 100, 75, 50, 25, 20, 15 or 10 amino acids in length. In some embodiments, an N-terminal fragment of a protein encoded by the essential gene is about 140 amino acids in length. In some embodiments, an N-terminal fragment of a protein encoded by the essential gene is about 130 amino acids in length. In some embodiments, an N-terminal fragment of a protein encoded by the essential gene is about 120 amino acids in length. In some embodiments, an N-terminal fragment includes an amino acid sequence that is encoded by a region of the endogenous coding sequence of the essential gene that spans the break. In some embodiments, an N-terminal fragment includes an amino acid sequence that is encoded by a region of the endogenous coding sequence within 1 exon of the essential gene. In some embodiments, an N-terminal fragment includes an amino acid sequence that is encoded by a region of the endogenous coding sequence within 2 exons of the essential gene. In some embodiments, an N-terminal fragment includes an amino acid sequence that is encoded by a region of the endogenous coding sequence within 3 exons of the essential gene. In some embodiments, an N-terminal fragment includes an amino acid sequence that is encoded by a region of the endogenous coding sequence within 4 exons of the essential gene. In some embodiments, an N-terminal fragment includes an amino acid sequence that is encoded by a region of the endogenous coding sequence within 5 exons of the essential gene.

In some embodiments, the exogenous partial coding sequence of an essential gene in a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette encodes a 20 amino acid N-terminal fragment of a protein encoded by an essential gene. In some embodiments, the exogenous partial coding sequence of an essential gene in a knock-in cassette encodes a 19 amino acid N-terminal fragment of a protein encoded by an essential gene. In some embodiments, the exogenous partial coding sequence of an essential gene in a knock-in cassette encodes an 18 amino acid N-terminal fragment of a protein encoded by an essential gene. In some embodiments, the exogenous partial coding sequence of an essential gene in a knock-in cassette encodes a 17 amino acid N-terminal fragment of a protein encoded by an essential gene. In some embodiments, the exogenous partial coding sequence of an essential gene in a knock-in cassette encodes a 16 amino acid N-terminal fragment of a protein encoded by an essential gene. In some embodiments, the exogenous partial coding sequence of an essential gene in a knock-in cassette encodes a 1 amino acid N-terminal fragment of a protein encoded by an essential gene.

In some embodiments, the exogenous coding sequence or partial coding sequence of the essential gene in the knock-in cassette is less than 100% identical to the corresponding endogenous coding sequence of the essential gene of the cell, e.g., less than 99%, 98%, 97%, 96%, 95%, 94%, 93%, 92%, 91%, 90%, 85%, 80%, 75%, 70%, 65%, 60%, 55% or less than 50% (i.e., when the two sequences are aligned using a standard pairwise sequence alignment tool that maximizes the alignment between the corresponding sequences). For example, in some embodiments, the exogenous coding sequence or partial coding sequence of the essential gene in the knock-in cassette is codon optimized relative to the corresponding endogenous coding sequence of the essential gene of the cell, e.g., to prevent further binding of a nuclease to the target site. Alternatively or additionally it may be codon optimized to reduce the likelihood of recombination after integration of the knock-in cassette into the genome of the cell and/or to increase expression of the gene product of the essential gene and/or the gene product of interest after integration of the knock-in cassette into the genome of the cell.

In some embodiments, a knock-in cassette comprises one or more nucleotides or base pairs that differ (e.g., are mutations) relative to an endogenous knock-in site. In some embodiments, such mutations in a knock-in cassette provide resistance to cutting by a nuclease. In some embodiments, such mutations in a knock-in cassette prevent a nuclease from cutting the target loci following homologous recombination. In some embodiments, such mutations in a knock-in cassette occur within one or more coding and/or non-coding regions of a target gene. In some embodiments, such mutations in a knock-in cassette are silent mutations. In some embodiments, such mutations in a knock-in cassette are silent and/or missense mutations.

In some embodiments, such mutations in a knock-in cassette occur within a target protospacer motif and/or a target protospacer adjacent motif (PAM) site. In some embodiments, a knock-in cassette includes a target protospacer motif and/or a PAM site that are saturated with silent mutations. In some embodiments, a knock-in cassette includes a target protospacer motif and/or a PAM site that are approximately 30%, 40%, 50%, 60%, 70%, 80%, or 90% saturated with silent mutations. In some embodiments, a knock-in cassette includes a target protospacer motif and/or a PAM site that are saturated with silent and/or missense mutations. In some embodiments, a knock-in cassette includes a target protospacer motif and/or a PAM site that comprise at least one mutation, at least 2 mutations, at least 3 mutations, at least 4 mutations, at least 5 mutations, at least 6 mutations, at least 7 mutations, at least 8 mutations, at least 9 mutations, at least 10 mutations, at least 11 mutations, at least 12 mutations, at least 13 mutations, at least 14 mutations, or at least 15 mutations.

In some embodiments, certain codons encoding certain amino acids in a target site cannot be mutated through codon-optimization without losing some portion of an endogenous proteins natural function. In some embodiments, certain codons encoding certain amino acids in a target site cannot be mutated through codon-optimization.

In some embodiments, the knock-in cassette is codon optimized in only a portion of the coding sequence. For example, in some embodiments, a knock-in cassette encodes a C-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid C-terminal fragment of a protein encoded by an essential gene. In some embodiments, the exogenous partial coding sequence of an essential gene in a knock-in cassette that has been codon optimized encodes a 19 amino acid C-terminal fragment of a protein encoded by an essential gene. In some embodiments, the exogenous partial coding sequence of an essential gene in a knock-in cassette that has been codon optimized encodes an 18 amino acid C-terminal fragment of a protein encoded by an essential gene. In some embodiments, the exogenous partial coding sequence of an essential gene in a knock-in cassette that has been codon optimized encodes a 17 amino acid C-terminal fragment of a protein encoded by an essential gene. In some embodiments, the exogenous partial coding sequence of an essential gene in a knock-in cassette that has been codon optimized encodes a 16 amino acid C-terminal fragment of a protein encoded by an essential gene. In some embodiments, the exogenous partial coding sequence of an essential gene in a knock-in cassette that has been codon optimized encodes a 15 amino acid C-terminal fragment of a protein encoded by an essential gene. In some embodiments, the exogenous partial coding sequence of an essential gene in a knock-in cassette that has been codon optimized encodes a 14 amino acid C-terminal fragment of a protein encoded by an essential gene. In some embodiments, the exogenous partial coding sequence of an essential gene in a knock-in cassette that has been codon optimized encodes a 13 amino acid C-terminal fragment of a protein encoded by an essential gene. In some embodiments, the exogenous partial coding sequence of an essential gene in a knock-in cassette that has been codon optimized encodes a 12 amino acid C-terminal fragment of a protein encoded by an essential gene. In some embodiments, the exogenous partial coding sequence of an essential gene in a knock-in cassette that has been codon optimized encodes a 11 amino acid C-terminal fragment of a protein encoded by an essential gene. In some embodiments, the exogenous partial coding sequence of an essential gene in a knock-in cassette that has been codon optimized encodes a 10 amino acid C-terminal fragment of a protein encoded by an essential gene. In some embodiments, the exogenous partial coding sequence of an essential gene in a knock-in cassette that has been codon optimized encodes a 9 amino acid C-terminal fragment of a protein encoded by an essential gene. In some embodiments, the exogenous partial coding sequence of an essential gene in a knock-in cassette that has been codon optimized encodes an 8 amino acid C-terminal fragment of a protein encoded by an essential gene. In some embodiments, the exogenous partial coding sequence of an essential gene in a knock-in cassette that has been codon optimized encodes a 7 amino acid C-terminal fragment of a protein encoded by an essential gene. In some embodiments, the exogenous partial coding sequence of an essential gene in a knock-in cassette that has been codon optimized encodes a 6 amino acid C-terminal fragment of a protein encoded by an essential gene. In some embodiments, the exogenous partial coding sequence of an essential gene in a knock-in cassette that has been codon optimized encodes a 5 amino acid C-terminal fragment of a protein encoded by an essential gene. In some embodiments, the exogenous partial coding sequence of an essential gene in a knock-in cassette that has been codon optimized encodes an amino acid C-terminal fragment that is less than 5 amino acids of a protein encoded by an essential gene.

In some embodiments, the knock-in cassette is codon optimized in only a portion of the coding sequence. For example, in some embodiments, a knock-in cassette encodes an N-terminal fragment of a protein encoded by an essential gene, e.g., a fragment that is less than 500, 250, 150, 125, 100, 75, 50, 25, 20, 19, 18, 17, 16, 15, 14, 13, 12, 11, 10, 9, 8, or 7 amino acids in length. In some embodiments, the exogenous partial coding sequence of an essential gene in a knock-in cassette that has been codon optimized encodes a 20 amino acid N-terminal fragment of a protein encoded by an essential gene. In some embodiments, the exogenous partial coding sequence of an essential gene in a knock-in cassette that has been codon optimized encodes a 19 amino acid N-terminal fragment of a protein encoded by an essential gene. In some embodiments, the exogenous partial coding sequence of an essential gene in a knock-in cassette that has been codon optimized encodes an 18 amino acid N-terminal fragment of a protein encoded by an essential gene. In some embodiments, the exogenous partial coding sequence of an essential gene in a knock-in cassette that has been codon optimized encodes a 17 amino acid N-terminal fragment of a protein encoded by an essential gene. In some embodiments, the exogenous partial coding sequence of an essential gene in a knock-in cassette that has been codon optimized encodes a 16 amino acid N-terminal fragment of a protein encoded by an essential gene. In some embodiments, the exogenous partial coding sequence of an essential gene in a knock-in cassette that has been codon optimized encodes a 15 amino acid N-terminal fragment of a protein encoded by an essential gene. In some embodiments, the exogenous partial coding sequence of an essential gene in a knock-in cassette that has been codon optimized encodes a 14 amino acid N-terminal fragment of a protein encoded by an essential gene. In some embodiments, the exogenous partial coding sequence of an essential gene in a knock-in cassette that has been codon optimized encodes a 13 amino acid N-terminal fragment of a protein encoded by an essential gene. In some embodiments, the exogenous partial coding sequence of an essential gene in a knock-in cassette that has been codon optimized encodes a 12 amino acid N-terminal fragment of a protein encoded by an essential gene. In some embodiments, the exogenous partial coding sequence of an essential gene in a knock-in cassette that has been codon optimized encodes a 11 amino acid N-terminal fragment of a protein encoded by an essential gene. In some embodiments, the exogenous partial coding sequence of an essential gene in a knock-in cassette that has been codon optimized encodes a 10 amino acid N-terminal fragment of a protein encoded by an essential gene. In some embodiments, the exogenous partial coding sequence of an essential gene in a knock-in cassette that has been codon optimized encodes a 9 amino acid N-terminal fragment of a protein encoded by an essential gene. In some embodiments, the exogenous partial coding sequence of an essential gene in a knock-in cassette that has been codon optimized encodes an 8 amino acid N-terminal fragment of a protein encoded by an essential gene. In some embodiments, the exogenous partial coding sequence of an essential gene in a knock-in cassette that has been codon optimized encodes a 7 amino acid N-terminal fragment of a protein encoded by an essential gene. In some embodiments, the exogenous partial coding sequence of an essential gene in a knock-in cassette that has been codon optimized encodes a 6 amino acid N-terminal fragment of a protein encoded by an essential gene. In some embodiments, the exogenous partial coding sequence of an essential gene in a knock-in cassette that has been codon optimized encodes a 5 amino acid N-terminal fragment of a protein encoded by an essential gene. In some embodiments, the exogenous partial coding sequence of an essential gene in a knock-in cassette that has been codon optimized encodes an amino acid N-terminal fragment that is less than 5 amino acids of a protein encoded by an essential gene.

In some embodiments, the knock-in cassette comprises one or more sequences encoding a linker peptide, e.g., between an exogenous coding sequence or partial coding sequence of the essential gene and a “cargo” sequence and/or a regulatory element described herein. Such linker peptides are known in the art, any of which can be included in a knock-in cassette described herein. In some embodiments, the linker peptide comprises the amino acid sequence GSG.

In some embodiments, the knock-in cassette comprises other regulatory elements such as a polyadenylation sequence, and optionally a 3′ UTR sequence, downstream of the exogenous coding sequence for the gene product of interest. If a 3′UTR sequence is present, the 3′UTR sequence is positioned 3′ of the exogenous coding sequence and 5′ of the polyadenylation sequence.

In some embodiments, the knock-in cassette comprises other regulatory elements such as a 5′ UTR and a start codon, upstream of the exogenous coding sequence for the gene product of interest. If a 5′UTR sequence is present, the 5′UTR sequence is positioned 5′ of the “cargo” sequence and/or exogenous coding sequence.

### Exemplary Homology Arms (HA)

In certain embodiments, a donor template comprises a 5′ and/or 3′ homology arm homologous to region of a GAPDH locus. In some embodiments, a donor template comprises a 5′ homology arm comprising or consisting of the sequence of SEQ ID NO: 1, 2, or 3. In some embodiments, a 5′ homology arm comprises or consists of a sequence that is at least 85%, 90%, 95%, 98% or 99% identical to the sequence of SEQ ID NO: 1, 2, or 3. In some embodiments, a donor template comprises a 3′ homology arm comprising or consisting of the sequence of SEQ ID NO:4 or 5. In certain embodiments, a 3′ homology arm comprises or consists of a sequence that is at least 85%, 90%, 95%, 98% or 99% identical to the sequence of SEQ ID NO: 4 or 5.

In some embodiments, a donor template comprises a 5′ homology arm comprising SEQ ID NO: 1, and a 3′ homology arm comprising SEQ ID NO: 4. In some embodiments, a donor template comprises a 5′ homology arm comprising SEQ ID NO: 2, and a 3′ homology arm comprising SEQ ID NO: 4. In some embodiments, a donor template comprises a 5′ homology arm comprising SEQ ID NO: 3, and a 3′ homology arm comprising SEQ ID NO: 5.

In some embodiments, a stretch of sequence flanking a nuclease cleavage site may be duplicated in both a 5′ and 3′ homology arm. In some embodiments, such a duplication is designed to optimize HDR efficiency. In some embodiments, one of the duplicated sequences may be codon optimized, while the other sequence is not codon optimized. In some embodiments, both of the duplicated sequences may be codon optimized. In some embodiments, codon optimization may remove a target PAM site. In some embodiments, a duplicated sequence may be no more than: 100 bp in length, 90 bp in length, 80 bp in length, 70 bp in length, 60 bp in length, 50 bp in length, 40 bp in length, 30 bp in length, or 20 bp in length.

exemplary 5′ HA for knock-in cassette
insertion at GAPDH locus
SEQ ID NO: 1
GAAGACTGTGGATGGCCCCTCCGGGAAACTGTGGCGTGATGGCCG
CGGGGCTCTCCAGAACATCATCCCTGCCTCTACTGGCGCTGCCAA
GGCTGTGGGCAAGGTCATCCCTGAGCTGAACGGGAAGCTCACTGG
CATGGCCTTCCGTGTCCCCACTGCCAACGTGTCAGTGGTGGACCT
GACCTGCCGTCTAGAAAAACCTGCCAAATATGATGACATCAAGAA
GGTGGTGAAGCAGGCGTCGGAGGGCCCCCTCAAGGGCATCCTGGG
CTACACTGAGCACCAGGTGGTCTCCTCTGACTTCAACAGCGACAC
CCACTCCTCCACCTTTGACGCTGGGGCTGGCATTGCCCTCAACGA
CCACTTTGTCAAGCTCATTTCCTGGTATGTGGCTGGGGCCAGAGA
CTGGCTCTTAAAAAGTGCAGGGTCTGGCGCCCTCTGGTGGCTGGC
TCAGAAAAAGGGCCCTGACAACTCTTTACATCTTCTAGGTATGAC
AACGAGTTCGGATATAGCAATAGAGTGGTCGATCTGATGGCTCAT
ATGGCTAGCAAAGAG
exemplary 5′ HA for knock-in cassette
insertion at GAPDH locus
SEQ ID NO: 2
GAAGACTGTGGATGGCCCCTCCGGGAAACTGTGGCGTGATGGCCG
CGGGGCTCTCCAGAACATCATCCCTGCCTCTACTGGCGCTGCCAA
GGCTGTGGGCAAGGTCATCCCTGAGCTGAACGGGAAGCTCACTGG
CATGGCCTTCCGTGTCCCCACTGCCAACGTGTCAGTGGTGGACCT
GACCTGCCGTCTAGAAAAACCTGCCAAATATGATGACATCAAGAA
GGTGGTGAAGCAGGCGTCGGAGGGCCCCCTCAAGGGCATCCTGGG
CTACACTGAGCACCAGGTGGTCTCCTCTGACTTCAACAGCGACAC
CCACTCCTCCACCTTTGACGCTGGGGCTGGCATTGCCCTCAACGA
CCACTTTGTCAAGCTCATTTCCTGGTATGTGGCTGGGGCCAGAGA
CTGGCTCTTAAAAAGTGCAGGGTCTGGCGCCCTCTGGTGGCTGGC
TCAGAAAAAGGGCCCTGACAACTCTTTACATCTTCTAGGTATGAC
AACGAGTTCGGATATAGCAATAGAGTGGTCGATCTGATGGCTCAT
ATGGCTAGCAAAGAGGGAAGCGGAGCTACTAACTTCAGCCTGCTG
AAGCAGGCTGGAGACGTGGAGGAGAACCCTGGACCT
exemplary 5′ HA for knock-in cassette
insertion at GAPDH locus
SEQ ID NO: 3
GGCTTTCCCATAATTTCCTTTCAAGGTGGGGAGGGAGGTAGAGGG
GTGATGTGGGGAGTACGCTGCAGGGCCTCACTCCTTTTGCAGACC
ACAGTCCATGCCATCACTGCCACCCAGAAGACTGTGGATGGCCCC
TCCGGGAAACTGTGGCGTGATGGCCGCGGGGCTCTCCAGAACATC
ATCCCTGCCTCTACTGGCGCTGCCAAGGCTGTGGGCAAGGTCATC
CCTGAGCTGAACGGGAAGCTCACTGGCATGGCCTTCCGTGTCCCC
ACTGCCAACGTGTCAGTGGTGGACCTGACCTGCCGTCTAGAAAAA
CCTGCCAAATATGATGACATCAAGAAGGTGGTGAAGCAGGCGTCG
GAGGGCCCCCTCAAGGGCATCCTGGGCTACACTGAGCACCAGGTG
GTCTCCTCTGACTTCAACAGCGACACCCACTCCTCCACCTTTGAC
GCTGGGGCTGGCATTGCCCTCAACGACCACTTTGTCAAGCTCATC
TCTTGGTACGACAATGAGTTCGGATATAGCAATAGAGTGGTCGAT
CTGATGGCTCATATGGCTAGCAAAGAG
exemplary 3′ HA for knock-in cassette
insertion at GAPDH locus
SEQ ID NO: 4
ATTTGGCTACAGCAACAGGGTGGTGGACCTCATGGCCCACATGGC
CTCCAAGGAGTAAGACCCCTGGACCACCAGCCCCAGCAAGAGCAC
AAGAGGAAGAGAGAGACCCTCACTGCTGGGGAGTCCCTGCCACAC
TCAGTCCCCCACCACACTGAATCTCCCCTCCTCACAGTTGCCATG
TAGACCCCTTGAAGAGGGGAGGGGCCTAGGGAGCCGCACCTTGTC
ATGTACCATCAATAAAGTACCCTGTGCTCAACCAGTTACTTGTCC
TGTCTTATTCTAGGGTCTGGGGCAGAGGGGAGGGAAGCTGGGCTT
GTGTCAAGGTGAGACATTCTTGCTGGGGAGGGACCTGGTATGTTC
TCCTCAGACTGAGGGTAGGGCCTCCAAACAGCCTTGCTTGCTTCG
AGAACCATTTGCTTCCCGCTCAGACGTCTTGAGTGCTACAGGAAG
CTGGCACCACTACTTCAGAGAACAAGGCCTTTTCCTCTCCTCGCT
CCAGT
exemplary 3′ HA for knock-in cassette
insertion at GAPDH locus
SEQ ID NO: 5
AGACTGGCTCTTAAAAAGTGCAGGGTCTGGCGCCCTCTGGTGGCT
GGCTCAGAAAAAGGGCCCTGACAACTCTTTTCATCTTCTAGGTAT
GACAACGAATTTGGCTACAGCAACAGGGTGGTGGACCTCATGGCC
CACATGGCCTCCAAGGAGTAAGACCCCTGGACCACCAGCCCCAGC
AAGAGCACAAGAGGAAGAGAGAGACCCTCACTGCTGGGGAGTCCC
TGCCACACTCAGTCCCCCACCACACTGAATCTCCCCTCCTCACAG
TTGCCATGTAGACCCCTTGAAGAGGGGAGGGGCCTAGGGAGCCGC
ACCTTGTCATGTACCATCAATAAAGTACCCTGTGCTCAACCAGTT
ACTTGTCCTGTCTTATTCTAGGGTCTGGGGCAGAGGGGAGGGAAG
CTGGGCTTGTGTCAAGGTGAGACATTCTTGCTGGGGAGGGACCTG
GTATGTTCTCCTCAGACTGAGGGTAGGGCCTCCAAACAGCCTTGC
TTGCT

In some embodiments, a donor template comprises a 5′ and/or 3′ homology arm homologous to a region of a TBP locus. In some embodiments, a donor template comprises a 5′ homology arm comprising or consisting of the sequence of SEQ ID NO:6, 7, or 8. In some embodiments, a 5′ homology arm comprises or consists of a sequence that is at least 85%, 90%, 95%, 98% or 99% identical to the sequence of SEQ ID NO: 6, 7, or 8. In some embodiments, a donor template comprises a 3′ homology arm comprising or consisting of the sequence of SEQ ID NO:9, 10, or 11. In certain embodiments, a 3′ homology arm comprises or consists of a sequence that is at least 85%, 90%, 95%, 98% or 99% identical to the sequence of SEQ ID NO: 9, 10, or 11.

In some embodiments, a donor template comprises a 5′ homology arm comprising SEQ ID NO: 6, and a 3′ homology arm comprising SEQ ID NO: 9. In some embodiments, a donor template comprises a 5′ homology arm comprising SEQ ID NO: 7, and a 3′ homology arm comprising SEQ ID NO: 10. In some embodiments, a donor template comprises a 5′ homology arm comprising SEQ ID NO: 8, and a 3′ homology arm comprising SEQ ID NO: 11.

exemplary 5′ HA for knock-in cassette
insertion at TBP locus
SEQ ID NO: 6
GCAGACTTCCATTTACAGTGAGGAGGTGAGCATTGCATTGAACAA
AAGATGGCGTTTTCACTTGGAATTAGTTATCTGAAGCTTTAGGAT
TCCTCAGCAATATGATTATGAGACAAGAAAGGAAGATTCAGAAAT
GAGTCTAGTTGAAGGCAGCAATTCAGAGAAGAAGATTCAGTTGTT
ATCATTGCCGTCCTGCTTGGTTTATGGCCTGGTTCAGGACCAAGG
AGAGAAGTGTGAATACATGCCTCTTGAGCTATAGAATGAGACGCT
GGAGTCACTAAGATGATTTTTTAAAAGTATTGTTTTATAAACAAA
AATAAGATTGTGACAAGGGATTCCACTATTAATGTTTTCATGCCT
GTGCCTTAATCTGACTGGGTATGGTGAGAATTGTGCTTGCAGCTT
TAAGGTAAGAATTTTACCATCTTAATATGTTAAGAAGTGCCATTT
CAGTCTCTCATCTCTACTCCAACTTGTCTTCTTAGGTGCTAAAGT
CAGAGCCGAAATCTACGAGGCCTTCGAGAACATCTACCCCATCCT
GAAGGGCTTCAGAAAGACCACC
exemplary 5′ HA for knock-in cassette
insertion at TBP locus
SEQ ID NO: 7
CTGACCACAGCTCTGCAAGCAGACTTCCATTTACAGTGAGGAGGT
GAGCATTGCATTGAACAAAAGATGGCGTTTTCACTTGGAATTAGT
TATCTGAAGCTTTAGGATTCCTCAGCAATATGATTATGAGACAAG
AAAGGAAGATTCAGAAATGAGTCTAGTTGAAGGCAGCAATTCAGA
GAAGAAGATTCAGTTGTTATCATTGCCGTCCTGCTTGGTTTATGG
CCTGGTTCAGGACCAAGGAGAGAAGTGTGAATACATGCCTCTTGA
GCTATAGAATGAGACGCTGGAGTCACTAAGATGATTTTTTAAAAG
TATTGTTTTATAAACAAAAATAAGATTGTGACAAGGGATTCCACT
ATTAATGTTTTCATGCCTGTGCCTTAATCTGACTGGGTATGGTGA
GAATTGTGCTTGCAGCTTTAAGGTAAGAATTTTACCATCTTAATA
TGTTAAGAAGTGCCATTTCAGTCTCTCATCTCTACTCCAACTTGT
CTTCTTAGGGGCTAAAGTGCGGGCCGAGATCTACGAGGCCTTCGA
GAATATCTACCCCATCCTGAAGGGCTTCAGAAAGACCACC
exemplary 5′ HA for knock-in cassette
insertion at TBP locus
SEQ ID NO: 8
ACAAAAGATGGCGTTTTCACTTGGAATTAGTTATCTGAAGCTTTA
GGATTCCTCAGCAATATGATTATGAGACAAGAAAGGAAGATTCAG
AAATGAGTCTAGTTGAAGGCAGCAATTCAGAGAAGAAGATTCAGT
TGTTATCATTGCCGTCCTGCTTGGTTTATGGCCTGGTTCAGGACC
AAGGAGAGAAGTGTGAATACATGCCTCTTGAGCTATAGAATGAGA
CGCTGGAGTCACTAAGATGATTTTTTAAAAGTATTGTTTTATAAA
CAAAAATAAGATTGTGACAAGGGATTCCACTATTAATGTTTTCAT
GCCTGTGCCTTAATCTGACTGGGTATGGTGAGAATTGTGCTTGCA
GCTTTAAGGTAAGAATTTTACCATCTTAATATGTTAAGAAGTGCC
ATTTCAGTCTCTCATCTCTACTCCAACTTGTCTTCTTAGGTGCTA
AAGTCAGAGCAGAAATTTATGAAGCATTCGAGAACATCTACCCTA
TTCTAAAGGGATTCAGGAAGACGACG
exemplary 3′ HA for knock-in cassette
insertion at TBP locus
SEQ ID NO: 9
CAGAAATTTATGAAGCATTTGAAAACATCTACCCTATTCTAAAGG
GATTCAGGAAGACGACGTAATGGCTCTCATGTACCCTTGCCTCCC
CCACCCCCTTCTTTTTTTTTTTTTAAACAAATCAGTTTGTTTTGG
TACCTTTAAATGGTGGTGTTGTGAGAAGATGGATGTTGAGTTGCA
GGGTGTGGCACCAGGTGATGCCCTTCTGTAAGTGCCCACCGCGGG
ATGCCGGGAAGGGGCATTATTTGTGCACTGAGAACACCGCGCAGC
GTGACTGTGAGTTGCTCATACCGTGCTGCTATCTGGGCAGCGCTG
CCCATTTATTTATATGTAGATTTTAAACACTGCTGTTGACAAGTT
GGTTTGAGGGAGAAAACTTTAAGTGTTAAAGCCACCTCTATAATT
GATTGGACTTTTTAATTTTAATGTTTTTCCCCATGAACCACAGTT
TTTATATTTCTACCAGAAAAGTAAAAATCTTTTTTAAAAGTGTTG
TTTTT
exemplary 3′ HA for knock-in cassette
insertion at TBP locus
SEQ ID NO: 10
TAGGTGCTAAAGTCAGAGCAGAAATTTATGAAGCATTTGAAAACA
TCTACCCTATTCTAAAGGGATTCAGGAAGACGACGTAATGGCTCT
CATGTACCCTTGCCTCCCCCACCCCCTTCTTTTTTTTTTTTTAAA
CAAATCAGTTTGTTTTGGTACCTTTAAATGGTGGTGTTGTGAGAA
GATGGATGTTGAGTTGCAGGGTGTGGCACCAGGTGATGCCCTTCT
GTAAGTGCCCACCGCGGGATGCCGGGAAGGGGCATTATTTGTGCA
CTGAGAACACCGCGCAGCGTGACTGTGAGTTGCTCATACCGTGCT
GCTATCTGGGCAGCGCTGCCCATTTATTTATATGTAGATTTTAAA
CACTGCTGTTGACAAGTTGGTTTGAGGGAGAAAACTTTAAGTGTT
AAAGCCACCTCTATAATTGATTGGACTTTTTAATTTTAATGTTTT
TCCCCATGAACCACAGTTTTTATATTTCTACCAGAAAAGTAAAAA
TCTTT
exemplary 3′ HA for knock-in cassette
insertion at TBP locus
SEQ ID NO: 11
AAGGGATTCAGGAAGACGACGTAATGGCTCTCATGTACCCTTGCC
TCCCCCACCCCCTTCTTTTTTTTTTTTTAAACAAATCAGTTTGTT
TTGGTACCTTTAAATGGTGGTGTTGTGAGAAGATGGATGTTGAGT
TGCAGGGTGTGGCACCAGGTGATGCCCTTCTGTAAGTGCCCACCG
CGGGATGCCGGGAAGGGGCATTATTTGTGCACTGAGAACACCGCG
CAGCGTGACTGTGAGTTGCTCATACCGTGCTGCTATCTGGGCAGC
GCTGCCCATTTATTTATATGTAGATTTTAAACACTGCTGTTGACA
AGTTGGTTTGAGGGAGAAAACTTTAAGTGTTAAAGCCACCTCTAT
AATTGATTGGACTTTTTAATTTTAATGTTTTTCCCCATGAACCAC
AGTTTTTATATTTCTACCAGAAAAGTAAAAATCTTTTTTAAAAGT
GTTGTTTTTCTAATTTATAACTCCTAGGGGTTATTTCTGTGCCAG
ACACA

In some embodiments, a donor template comprises a 5′ and/or 3′ homology arm homologous to a region of a G6PD locus. In some embodiments, a donor template comprises a 5′ homology arm comprising or consisting of the sequence of SEQ ID NO:12. In some embodiments, a 5′ homology arm comprises or consists of a sequence that is at least 85%, 90%, 95%, 98% or 99% identical to the sequence of SEQ ID NO: 12. In some embodiments, a donor template comprises a 3′ homology arm comprising or consisting of the sequence of SEQ ID NO: 13. In certain embodiments, a 3′ homology arm comprises or consists of a sequence that is at least 85%, 90%, 95%, 98% or 99% identical to the sequence of SEQ ID NO: 13.

In some embodiments, a donor template comprises a 5′ homology arm comprising SEQ ID NO: 12, and a 3′ homology arm comprising SEQ ID NO: 13.

exemplary 5′ HA for knock-in cassette
insertion at G6PD locus
SEQ ID NO: 12
GGCCCGGGGGACTCCACATGGTGGCAGGCAGTGGCATCAGCAAGA
CACTCTCTCCCTCACAGAACGTGAAGCTCCCTGACGCCTATGAGC
GCCTCATCCTGGACGTCTTCTGCGGGAGCCAGATGCACTTCGTGC
GCAGGTGAGGCCCAGCTGCCGGCCCCTGCATACCTGTGGGCTATG
GGGTGGCCTTTGCCCTCCCTCCCTGTGTGCCACCGGCCTCCCAAG
CCATACCATGTCCCCTCAGCGACGAGCTCCGTGAGGCCTGGCGTA
TTTTCACCCCACTGCTGCACCAGATTGAGCTGGAGAAGCCCAAGC
CCATCCCCTATATTTATGGCAGGTGAGGAAAGGGTGGGGGCTGGG
GACAGAGCCCAGCGGGCAGGGGCGGGGTGAGGGTGGAGCTACCTC
ATGCCTCTCCTCCACCCGTCACTCTCCAGCCGAGGCCCCACGGAG
GCAGACGAGCTGATGAAGAGAGTGGGCTTCCAGTACGAGGGAACC
TACAAATGGGTCAACCCTCACAAGCTG
exemplary 3′ HA for knock-in cassette
insertion at G6PD locus
SEQ ID NO: 13
GTGGGTGAACCCCCACAAGCTCTGAGCCCTGGGCACCCACCTCCA
CCCCCGCCACGGCCACCCTCCTTCCCGCCGCCCGACCCCGAGTCG
GGAGGACTCCGGGACCATTGACCTCAGCTGCACATTCCTGGCCCC
GGGCTCTGGCCACCCTGGCCCGCCCCTCGCTGCTGCTACTACCCG
AGCCCAGCTACATTCCTCAGCTGCCAAGCACTCGAGACCATCCTG
GCCCCTCCAGACCCTGCCTGAGCCCAGGAGCTGAGTCACCTCCTC
CACTCACTCCAGCCCAACAGAAGGAAGGAGGAGGGCGCCCATTCG
TCTGTCCCAGAGCTTATTGGCCACTGGGTCTCACTCCTGAGTGGG
GCCAGGGTGGGAGGGAGGGACGAGGGGGAGGAAAGGGGCGAGCAC
CCACGTGAGAGAATCTGCCTGTGGCCTTGCCCGCCAGCCTCAGTG
CCACTTGACATTCCTTGTCACCAGCAACATCTCGAGCCCCCTGGA
TGTCC

In some embodiments, a donor template comprises a 5′ and/or 3′ homology arm homologous to a region of a E2F4 locus. In some embodiments, a donor template comprises a 5′ homology arm comprising or consisting of the sequence of SEQ ID NO: 14, 15, or 16. In some embodiments, a 5′ homology arm comprises or consists of a sequence that is at least 85%, 90%, 95%, 98% or 99% identical to the sequence of SEQ ID NO: 14, 15, or 16. In some embodiments, a donor template comprises a 3′ homology arm comprising or consisting of the sequence of SEQ ID NO: 17, 18, or 19. In certain embodiments, a 3′ homology arm comprises or consists of a sequence that is at least 85%, 90%, 95%, 98% or 99% identical to the sequence of SEQ ID NO: 17, 18, or 19.

In some embodiments, a donor template comprises a 5′ homology arm comprising SEQ ID NO: 14, and a 3′ homology arm comprising SEQ ID NO: 17. In some embodiments, a donor template comprises a 5′ homology arm comprising SEQ ID NO: 15, and a 3′ homology arm comprising SEQ ID NO: 18. In some embodiments, a donor template comprises a 5′ homology arm comprising SEQ ID NO: 16, and a 3′ homology arm comprising SEQ ID NO: 19.

exemplary 5′ HA for knock-in cassette
insertion at E2F4 locus
SEQ ID NO: 14
CCAGGGGGCTGTAGTGGGGCCAGGCTGGACCTCTGTGCCCTGAGC
ATGGCTTTCTTGTTTTTCAGTTTTGGAACTCCCCAAAGAGCTGTC
AGAAATCTTTGATCCCACACGAGGTAGGCTGCTGCATTCCTCCCT
GAGGCTAGGGGTAAGGGACACAGCTCATTGGGTCCTATGGCTGTT
TTCTTGCCCTTTTGAGGACCTTGTTGTGGCGCTTATGGTAACTGG
GGCAAAGGGTGAAGTTCCTGATGGGCAGGTGGGGTTCCCTTTCCT
GGGCTTTGGTGGGTGGAGAGGTGGGAGCTGGAATGTTAGTAACTG
AGCTCCCTCCATTCCCAGAGTGCATGAGCTCGGAGCTGCTGGAGG
AGTTGATGTCCTCAGAAGGTGGGTGGCCCTGGAAGGTGGGAGTGG
GTGTGGGCAGGGGTTGGGCTGCTGCTAGGGGAGCCCTGGCCCAGG
GCCTGAGACTAGTGCTCTCTGCAGTGTTCGCCCCTCTGCTGAGAC
TTTCTCCTCCTCCTGGCGACCACGACTACATCTACAACCTGGACG
AGAGCGAGGGCGTGTGCGACCTGTTTGATGTGCCCGTGCTGAACC
TG
exemplary 5′ HA for knock-in cassette
insertion at E2F4 locus
SEQ ID NO: 15
CCAGGCTGGACCTCTGTGCCCTGAGCATGGCTTTCTTGTTTTTCA
GTTTTGGAACTCCCCAAAGAGCTGTCAGAAATCTTTGATCCCACA
CGAGGTAGGCTGCTGCATTCCTCCCTGAGGCTAGGGGTAAGGGAC
ACAGCTCATTGGGTCCTATGGCTGTTTTCTTGCCCTTTTGAGGAC
CTTGTTGTGGCGCTTATGGTAACTGGGGCAAAGGGTGAAGTTCCT
GATGGGCAGGTGGGGTTCCCTTTCCTGGGCTTTGGTGGGTGGAGA
GGTGGGAGCTGGAATGTTAGTAACTGAGCTCCCTCCATTCCCAGA
GTGCATGAGCTCGGAGCTGCTGGAGGAGTTGATGTCCTCAGAAGG
TGGGTGGCCCTGGAAGGTGGGAGTGGGTGTGGGCAGGGGTTGGGC
TGCTGCTAGGGGAGCCCTGGCCCAGGGCCTGAGACTAGTGCTCTC
TGCAGTGTTTGCCCCTCTGCTTCGTCTTAGTCCTCCTCCGGGCGA
CCACGACTACATCTACAACCTGGACGAGAGCGAGGGCGTGTGCGA
CCTGTTTGATGTGCCCGTGCTGAACCTG
exemplary 5′ HA for knock-in cassette
insertion at E2F4 locus
SEQ ID NO: 16
GTCAGAAATCTTTGATCCCACACGAGGTAGGCTGCTGCATTCCTC
CCTGAGGCTAGGGGTAAGGGACACAGCTCATTGGGTCCTATGGCT
GTTTTCTTGCCCTTTTGAGGACCTTGTTGTGGCGCTTATGGTAAC
TGGGGCAAAGGGTGAAGTTCCTGATGGGCAGGTGGGGTTCCCTTT
CCTGGGCTTTGGTGGGTGGAGAGGTGGGAGCTGGAATGTTAGTAA
CTGAGCTCCCTCCATTCCCAGAGTGCATGAGCTCGGAGCTGCTGG
AGGAGTTGATGTCCTCAGAAGGTGGGTGGCCCTGGAAGGTGGGAG
TGGGTGTGGGCAGGGGTTGGGCTGCTGCTAGGGGAGCCCTGGCCC
AGGGCCTGAGACTAGTGCTCTCTGCAGTGTTTGCCCCTCTGCTTC
GTCTTTCTCCACCCCCGGGAGACCACGATTATATCTACAACCTGG
ACGAGAGTGAAGGTGTCTGTGACCTCTTCGACGTGCCCGTGCTCA
ACCTC
exemplary 3′ HA for knock-in cassette
insertion at E2F4 locus
SEQ ID NO: 17
CCACCCCCGGGAGACCACGATTATATCTACAACCTGGACGAGAGT
GAAGGTGTCTGTGACCTCTTTGATGTGCCTGTTCTCAACCTCTGA
CTGACAGGGACATGCCCTGTGTGGCTGGGACCCAGACTGTCTGAC
CTGGGGGTTGCCTGGGGACCTCTCCCACCCGACCCCTACAGAGCT
TGAGAGCCACAGACGCCTGGCTTCTCCGGCCTCCCCTCACCGCAC
AGTTCTGGCCACAGCTCCCGCTCCTGTGCTGGCACTTCTGTGCTC
GCAGAGCAGGGGAACAGGACTCAGCCCCCATCACCGTGGAGCCAA
AGTGTTTGCTTCTCCCTTTCTGCGGCCTTCGCCAGCCCAGGCTCG
GCTGCCACCCAGTGGCACAGAACCGAGGAGCTGCCATTACCCCCC
ATAGGGGGCAGTGTCTTGTTCCTGCCAGCCTCAGTGTCTTGCTTC
TGCCAGCTCCTTCCCCTAGGAGGGAAGGGTGGGGTGGAACTGGGC
ACATG
exemplary 3′ HA for knock-in cassette
insertion at E2F4 locus
SEQ ID NO: 18
ATTATATCTACAACCTGGACGAGAGTGAAGGTGTCTGTGACCTCT
TTGATGTGCCTGTTCTCAACCTCTGACTGACAGGGACATGCCCTG
TGTGGCTGGGACCCAGACTGTCTGACCTGGGGGTTGCCTGGGGAC
CTCTCCCACCCGACCCCTACAGAGCTTGAGAGCCACAGACGCCTG
GCTTCTCCGGCCTCCCCTCACCGCACAGTTCTGGCCACAGCTCCC
GCTCCTGTGCTGGCACTTCTGTGCTCGCAGAGCAGGGGAACAGGA
CTCAGCCCCCATCACCGTGGAGCCAAAGTGTTTGCTTCTCCCTTT
CTGCGGCCTTCGCCAGCCCAGGCTCGGCTGCCACCCAGTGGCACA
GAACCGAGGAGCTGCCATTACCCCCCATAGGGGGCAGTGTCTTGT
TCCTGCCAGCCTCAGTGTCTTGCTTCTGCCAGCTCCTTCCCCTAG
GAGGGAAGGGTGGGGTGGAACTGGGCACATGCCAGCACCACTTCT
AGCTT
exemplary 3′ HA for knock-in cassette
insertion at E2F4 locus
SEQ ID NO: 19
TGACTGACAGGGACATGCCCTGTGTGGCTGGGACCCAGACTGTCT
GACCTGGGGGTTGCCTGGGGACCTCTCCCACCCGACCCCTACAGA
GCTTGAGAGCCACAGACGCCTGGCTTCTCCGGCCTCCCCTCACCG
CACAGTTCTGGCCACAGCTCCCGCTCCTGTGCTGGCACTTCTGTG
CTCGCAGAGCAGGGGAACAGGACTCAGCCCCCATCACCGTGGAGC
CAAAGTGTTTGCTTCTCCCTTTCTGCGGCCTTCGCCAGCCCAGGC
TCGGCTGCCACCCAGTGGCACAGAACCGAGGAGCTGCCATTACCC
CCCATAGGGGGCAGTGTCTTGTTCCTGCCAGCCTCAGTGTCTTGC
TTCTGCCAGCTCCTTCCCCTAGGAGGGAAGGGTGGGGTGGAACTG
GGCACATGCCAGCACCACTTCTAGCTTCCTTCGCTATCCCCCACC
CCCTGACCCTCCAGCTCCTCCTGGCCCTCTCACGTGCCCACTTCT
GCTGG

In some embodiments, a donor template comprises a 5′ and/or 3′ homology arm homologous to a region of a KIF11 locus. In some embodiments, a donor template comprises a 5′ homology arm comprising or consisting of the sequence of SEQ ID NO: 20, 21, or 22. In some embodiments, a 5′ homology arm comprises or consists of a sequence that is at least 85%, 90%, 95%, 98% or 99% identical to the sequence of SEQ ID NO: 20, 21, or 22. In some embodiments, a donor template comprises a 3′ homology arm comprising or consisting of the sequence of SEQ ID NO: 23, 24, or 25. In certain embodiments, a 3′ homology arm comprises or consists of a sequence that is at least 85%, 90%, 95%, 98% or 99% identical to the sequence of SEQ ID NO: 23, 24, or 25.

In some embodiments, a donor template comprises a 5′ homology arm comprising SEQ ID NO: 20, and a 3′ homology arm comprising SEQ ID NO: 23. In some embodiments, a donor template comprises a 5′ homology arm comprising SEQ ID NO: 21, and a 3′ homology arm comprising SEQ ID NO: 24. In some embodiments, a donor template comprises a 5′ homology arm comprising SEQ ID NO: 22, and a 3′ homology arm comprising SEQ ID NO: 25.

exemplary 5′ HA for knock-in cassette
insertion at KIF11 locus
SEQ ID NO: 20
AGAGCAGGGTTTCTTGACAGCAGTGCTATTGGCATTTTAAACTGG
ATAATTCTTTGTTGTGATGGGCTTTCCTGTGGACTGTACTATGTT
GGTACACAAGAAAAACAGTGTACTATGTGAATACTCACTCAAAGC
CAGTAGCACTCCCTGATTGTAACACCAAAAAAGTCTCTCAGCATT
GCCAAATGTCCCCTGTGGCAGCAGAATCACTCCCTGATGAGAACC
ACTACCCTGGAGTAAAATCTATAACTATGTCTTAGAAAATAACAC
AGAAAATTAATATTTCTTTCACTCTACTCCTTCCATTAGTGATCA
AATAAAGAAGGCATTTGGCGCTACTTGCCAAATTGTTGGCTCAAA
CTTGTGCTGAACCTTTTTTGGTTTTCTACACTTAAGTTTTTTTGC
CTATAACCCAGAGAACTTTGAAAATAGAGTGTAGTTAATGTGTAT
CTAATGTTACTTTGTATTGACTTAATTTACCGGCCTTTAATCCAC
AGCATAAGAAGTCCCACGGCAAGGACAAAGAGAACCGGGGCATCA
ACACACTGGAACGGTCCAAGGTCGAGGAAACAACCGAGCACCTGG
TCACCAAGAGCAGACTGCCTCTGAGAGCCCAGATCAACCTG
exemplary 5′ HA for knock-in cassette
insertion at KIF11 locus
SEQ ID NO: 21
TTCCTGTGGACTGTACTATGTTGGTACACAAGAAAAACAGTGTAC
TATGTGAATACTCACTCAAAGCCAGTAGCACTCCCTGATTGTAAC
ACCAAAAAAGTCTCTCAGCATTGCCAAATGTCCCCTGTGGCAGCA
GAATCACTCCCTGATGAGAACCACTACCCTGGAGTAAAATCTATA
ACTATGTCTTAGAAAATAACACAGAAAATTAATATTTCTTTCACT
CTACTCCTTCCATTAGTGATCAAATAAAGAAGGCATTTGGCGCTA
CTTGCCAAATTGTTGGCTCAAACTTGTGCTGAACCTTTTTTGGTT
TTCTACACTTAAGTTTTTTTGCCTATAACCCAGAGAACTTTGAAA
ATAGAGTGTAGTTAATGTGTATCTAATGTTACTTTGTATTGACTT
AATTTTCCCGCCTTAAATCCACAGCATAAAAAATCACATGGAAAA
GACAAAGAAAACAGAGGCATTAACACACTGGAGAGGTCTAAAGTG
GAAGAAACAACCGAGCACCTGGTCACCAAGAGCAGACTGCCTCTG
AGAGCCCAGATCAACCTG
exemplary 5′ HA for knock-in cassette
insertion at KIF11 locus
SEQ ID NO: 22
TTAAACTGGATAATTCTTTGTTGTGATGGGCTTTCCTGTGGACTG
TACTATGTTGGTACACAAGAAAAACAGTGTACTATGTGAATACTC
ACTCAAAGCCAGTAGCACTCCCTGATTGTAACACCAAAAAAGTCT
CTCAGCATTGCCAAATGTCCCCTGTGGCAGCAGAATCACTCCCTG
ATGAGAACCACTACCCTGGAGTAAAATCTATAACTATGTCTTAGA
AAATAACACAGAAAATTAATATTTCTTTCACTCTACTCCTTCCAT
TAGTGATCAAATAAAGAAGGCATTTGGCGCTACTTGCCAAATTGT
TGGCTCAAACTTGTGCTGAACCTTTTTTGGTTTTCTACACTTAAG
TTTTTTTGCCTATAACCCAGAGAACTTTGAAAATAGAGTGTAGTT
AATGTGTATCTAATGTTACTTTGTATTGACTTAATTTTCCCGCCT
TAAATCCACAGCATAAAAAATCACATGGAAAAGACAAAGAAAACA
GAGGCATCAACACACTGGAACGGTCCAAGGTCGAGGAAACAACCG
AGCACCTGGTCACCAAGAGCAGACTGCCTCTGAGAGCCCAGATCA
ACCTG
exemplary 3′ HA for knock-in cassette
insertion at KIF11 locus
SEQ ID NO: 23
AAAAAATCACATGGAAAAGACAAAGAAAACAGAGGCATTAACACA
CTGGAGAGGTCTAAAGTGGAAGAAACTACAGAGCACTTGGTTACA
AAGAGCAGATTACCTCTGCGAGCCCAGATCAACCTTTAATTCACT
TGGGGGTTGGCAATTTTATTTTTAAAGAAAACTTAAAAATAAAAC
CTGAAACCCCAGAACTTGAGCCTTGTGTATAGATTTTAAAAGAAT
ATATATATCAGCCGGGCGCGGTGGCTCATGCCTGTAATCCCAGCA
CTTTGGGAGGCTGAGGCGGGTGGATTGCTTGAGCCCAGGAGTTTG
AGACCAGCCTGGCCAACGTGGCAAAACCTCGTCTCTGTTAAAAAT
TAGCCGGGCGTGGTGGCACACTCCTGTAATCCCAGCTACTGGGGA
GGCTGAGGCACGAGAATCACTTGAACCCAGGAAGCGGGGTTGCAG
TGAGCCAAAGGTACACCACTACACTCCAGCCTGGGCAACAGAGCA
AGACT
exemplary 3′ HA for knock-in cassette
insertion at KIF11 locus
SEQ ID NO: 24
AACTACAGAGCACTTGGCTACATAGAGCAGATTACCTCTGCGAGC
CCAGATCAACCTTTAATTCACTTGGGGGTTGGCAATTTTATTTTT
AAAGAAAACTTAAAAATAAAACCTGAAACCCCAGAACTTGAGCCT
TGTGTATAGATTTTAAAAGAATATATATATCAGCCGGGCGCGGTG
GCTCATGCCTGTAATCCCAGCACTTTGGGAGGCTGAGGCGGGTGG
ATTGCTTGAGCCCAGGAGTTTGAGACCAGCCTGGCCAACGTGGCA
AAACCTCGTCTCTGTTAAAAATTAGCCGGGCGTGGTGGCACACTC
CTGTAATCCCAGCTACTGGGGAGGCTGAGGCACGAGAATCACTTG
AACCCAGGAAGCGGGGTTGCAGTGAGCCAAAGGTACACCACTACA
CTCCAGCCTGGGCAACAGAGCAAGACTCGGTCTCAAAAACAAAAT
TTAAAAAAGATATAAGGCAGTACTGTAAATTCAGTTGAATTTTGA
TATCT
exemplary 3′ HA for knock-in cassette
insertion at KIF11 locus
SEQ ID NO: 25
ATTAACACACTGGAGAGTTCTGAAGTGGAAGAAACTACAGAGCAC
TTGGTTACAAAGAGCAGATTACCTCTGCGAGCCCAGATCAACCTT
TAATTCACTTGGGGGTTGGCAATTTTATTTTTAAAGAAAACTTAA
AAATAAAACCTGAAACCCCAGAACTTGAGCCTTGTGTATAGATTT
TAAAAGAATATATATATCAGCCGGGCGCGGTGGCTCATGCCTGTA
ATCCCAGCACTTTGGGAGGCTGAGGCGGGTGGATTGCTTGAGCCC
AGGAGTTTGAGACCAGCCTGGCCAACGTGGCAAAACCTCGTCTCT
GTTAAAAATTAGCCGGGCGTGGTGGCACACTCCTGTAATCCCAGC
TACTGGGGAGGCTGAGGCACGAGAATCACTTGAACCCAGGAAGCG
GGGTTGCAGTGAGCCAAAGGTACACCACTACACTCCAGCCTGGGC
AACAGAGCAAGACTCGGTCTCAAAAACAAAATTTAAAAAAGATAT
AAGGC

### Inverted Terminal Repeats (ITRs)

In certain embodiments, a donor template comprises an AAV derived sequence. In certain embodiments, a donor template comprises AAV derived sequences that are typical of an AAV construct, such as cis-acting 5′ and 3′ inverted terminal repeats (ITRs) (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence) into a genome of a target cell. ITRs can also aid in efficient encapsidation of an AAV construct in an AAV particle.

In some embodiments, a donor template described herein is included within an rAAV particle (e.g., an AAV6 particle). In some embodiments, an ITR is or comprises about 145 nucleic acids. In some embodiments, all or substantially all of a sequence encoding an ITR is used. In some embodiments, an AAV ITR sequence may be obtained from any known AAV, including presently identified mammalian AAV types. In some embodiments an ITR is an AAV6 ITR.

An example of an AAV construct employed in the present disclosure is a “cis-acting” construct containing a cargo sequence (e.g., a donor template described herein), in which the donor template is flanked by 5′ or “left” and 3′ or “right” AAV ITR sequences. 5′ and left designations refer to a position of an ITR sequence relative to an entire construct, read left to right, in a sense direction. For example, in some embodiments, a 5′ or left ITR is an ITR that is closest to a target loci promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some embodiments, a 3′ or right ITR is an ITR that is closest to a polyadenylation sequence in a target loci (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some embodiments an ITR (e.g., a 5′ ITR) can have a sequence according to SEQ ID NO: 158. In some embodiments, an ITR (e.g., a 3′ ITR) can have a sequence according to SEQ ID NO: 159. In some embodiments, an ITR includes one or more modifications, e.g., truncations, deletions, substitutions or insertions, as is known in the art. In some embodiments, an ITR comprises fewer than 145 nucleotides, e.g., 127, 130, 134 or 141 nucleotides. For example, in some embodiments, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

A non-limiting example of 5′ AAV ITR sequences includes SEQ ID NO: 158. A non-limiting example of 3′ AAV ITR sequences includes SEQ ID NO: 159. In some embodiments, the 5′ and a 3′ AAV ITRs (e.g., SEQ ID NO: 158 and 159) flank a donor template described herein (e.g., a donor template comprising a 5′HA, a knock-in cassette, and a 3′ HA). The ability to modify ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some embodiments, a 5′ ITR sequence is at least 85%, 90%, 95%, 98% or 99% identical to a 5′ ITR sequence represented by SEQ ID NO: 158. In some embodiments, a 3′ ITR sequence is at least 85%, 90%, 95%, 98% or 99% identical to a 3′ ITR sequence represented by SEQ ID NO: 159.

exemplary 5′ ITR for knock-in cassette
insertion
SEQ ID NO: 158
CCTGCAGGCAGCTGCGCGCTCGCTCGCTCACTGAGGCCGCCCGGG
CAAAGCCCGGGCGTCGGGCGACCTTTGGTCGCCCGGCCTCAGTGA
GCGAGCGAGCGCGCAGAGAGGGAGTGGCCAACTCCATCACTAGGG
GTTCCT
exemplary 3′ ITR for knock-in cassette
insertion
SEQ ID NO: 159
AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGCTGCC
TGCAGG

### Flanking Untranslated Regions, 5′ UTRs and 3′ UTRs

In some embodiments, a knock-in cassette described herein includes all or a portion of an untranslated region (UTR), such as a 5′ UTR and/or a 3′ UTR. UTRs of a gene are transcribed but not translated. A 5′ UTR starts at a transcription start site and continues to the start codon but does not include the start codon. A 3′ UTR starts immediately following the stop codon and continues until the transcriptional termination signal. The regulatory and/or control features of a UTR can be incorporated into any of the knock-in cassettes described herein to enhance or otherwise modulate the expression of an essential target gene loci and/or a cargo sequence.

Natural 5′ UTRs include a sequence that plays a role in translation initiation. In some embodiments, a 5′ UTR comprises sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding. Non-limiting examples of 5′ UTRs include those from the following genes: albumin, serum amyloid A, Apolipoprotein A/B/E, transferrin, alpha fetoprotein, erythropoietin, and Factor VIII.

In some embodiments, a UTR may comprise a non-endogenous regulatory region. In some embodiments, a UTR that comprises a non-endogenous regulatory region is a 3′ UTR. In some embodiments, a UTR that comprises a non-endogenous regulatory region is a 5′ UTR. In some embodiments, a non-endogenous regulatory region may be a target of at least one inhibitory nucleic acid. In some embodiments, an inhibitory nucleic acid inhibits expression and/or activity of a target gene. In some embodiments, an inhibitory nucleic acid is a short interfering RNA (siRNA), a short hairpin RNA (shRNA), a microRNA (miRNA), an antisense oligonucleotide, a guide RNA (gRNA), or a ribozyme. In some embodiments, an inhibitory nucleic acid is an endogenous molecule. In some embodiments, an inhibitory nucleic acid is a non-endogenous molecule. In some embodiments, an inhibitory nucleic acid displays a tissue specific expression pattern. In some embodiments, an inhibitory nucleic acid displays a cell specific expression pattern.

In some embodiments, a knock-in cassette may comprise more than one non-endogenous regulatory regions, e.g., two, three, four, five, six, seven, eight, nine, or ten regulatory regions. In some embodiments, a knock-in cassette may comprise four non-endogenous regulatory regions. In some embodiments, a construct may comprise more than one non-endogenous regulatory regions, wherein at least one of the more than one non-endogenous regulatory regions are not the same as at least one of the other non-endogenous regulatory regions.

In some embodiments, a 3′ UTR is found immediately 3′ to the stop codon of a gene of interest. In some embodiments, a 3′ UTR from an mRNA that is transcribed by a target cell can be included in any knock-in cassette described herein. In some embodiments, a 3′ UTR is derived from an endogenous target loci and may include all or part of the endogenous sequence. In some embodiments, a 3′ UTR sequence is at least 85%, 90%, 95% or 98% identical to the sequence of SEQ ID NO: 26.

exemplary 3′ UTR for knock-in cassette insertion
SEQ ID NO: 26
GCGGCCGCGTCGAGTCTAGAGGGCCCGTTTAAACCCGCTGATCAGCCTCGA

### Polyadenylation Sequences

In some embodiments, a knock-in cassette construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some embodiments, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad. Sci. U.S.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood 71(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some embodiments, a poly(A) signal sequence can be a synthetic polyadenylation site (see, e.g., the pCI-neo expression construct of Promega that is based on Levitt et al., Genes Dev. 3(7):1019-1025, 1989, which is incorporated herein by reference in its entirety). In some embodiments, a poly(A) signal sequence is the polyadenylation signal of soluble neuropilin-1 (sNRP) (AAATAAAATACGAAATG) (see, e.g., WO 05/073384, which is incorporated herein by reference in its entirety). In some embodiments, a poly(A) signal sequence comprises or consists of the SV40 poly(A) site. In some embodiments, a poly(A) signal sequence comprises or consists of SEQ ID NO: 27. In some embodiments, a poly(A) signal sequence comprises or consists of bGHpA. In some embodiments, a poly(A) signal sequence comprises or consists of SEQ ID NO: 28. Additional examples of poly(A) signal sequences are known in the art. In some embodiments, a poly(A) sequence is at least 85%, 90%, 95%, 98% or 99% identical to the sequence of SEQ ID NOs: 27 or 28.

exemplary SV40 poly(A) signal sequence
SEQ ID NO: 27
AACTTGTTTATTGCAGCTTATAATGGTTACAAATAAAGCAATAGCATCA
CAAATTTCACAAATAAAGCATTTTTTTCACTGCATTCTAGTTGTGGTTT
GTCCAAACTCATCAATGTATCTTA
exemplary bGH poly(A) signal sequence
SEQ ID NO: 28
CTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAAT
GAGGAAATTGCATCGCATTGTCTGAGTAGGTGTCATTCTATTCTGGGGG
GTGGGGTGGGGCAGGACAGCAAGGGGGAGGATTGGGAAGACAATAGCAG
GCATGCTGGGGATGCGGTGGGCTCTATGG

### IRES and 2A Elements

In some embodiments, the knock-in cassette comprises a regulatory element that enables expression of the gene product encoded by the essential gene and the gene product of interest as separate gene products, e.g., an IRES or 2A element located between the exogenous coding sequence or partial coding sequence of the essential gene and the exogenous coding sequence for the gene product of interest.

In some embodiments, a knock-in cassette may comprise multiple gene products of interest (e.g., at least two gene products of interest). In some embodiments, gene products of interest may be separated by a regulatory element that enables expression of the at least two gene products of interest as more than one gene product, e.g., an IRES or 2A element located between the at least two coding sequences, facilitating creation of at least two peptide products.

Internal Ribosome Entry Site (IRES) elements are one type of regulatory element that are commonly used for this purpose. As is well known in the art, IRES elements allow for initiation of translation from an internal region of the mRNA and hence expression of two separate proteins from the same mRNA transcript. IRES was originally discovered in poliovirus RNA, where it promotes translation of the viral genome in eukaryotic cells. Since then, a variety of IRES sequences have been discovered—many from viruses, but also some from cellular mRNAs, e.g., see Mokrejs et al., Nucleic Acids Res. 2006; 34(Database issue):D125-D130.

2A elements are another type of regulatory element that are commonly used for this purpose. These 2A elements encode so-called “self-cleaving” 2A peptides which are short peptides (about 20 amino acids) that were first discovered in picornaviruses. The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine (G) and Proline (P) residues found on the C-terminus meaning the upstream cistron, i.e., protein encoded by the essential gene will have a few additional residues from the 2A peptide added to the end, while the downstream cistron, i.e., gene product of interest will start with the Proline (P).

Table 2 below lists the four commonly used 2A peptides (an optional GSG sequence is sometimes added to the N-terminal end of the peptide to improve cleavage efficiency). There are many potential 2A peptides that may be suitable for methods and compositions described herein (see e.g., Luke et al., Occurrence, function and evolutionary origins of ‘2A-like’ sequences in virus genomes. J Gen Virol. 2008). Those skilled in the art know that the choice of specific 2A peptide for a particular knock-in cassette will ultimately depend on a number of factors such as cell type or experimental conditions. Those skilled in the art will recognize that nucleotide sequences encoding specific 2A peptides can vary while still encoding a peptide suitable for inducing a desired cleavage event.

TABLE 2
Exemplary IRES and 2A peptide and nucleic acid sequences

| SEQ ID NO: | 2A peptide | Amino acid sequence |
|---|---|---|
| 29 | T2A | EGRGSLLTCGDVEENPGP |
| 30 | P2A | ATNFSLLKQAGDVEENPGP |
| 31 | E2A | QCTNYALLKLAGDVESNPGP |
| 32 | F2A | VKQTLNFDLLKLAGDVESNPGP |
| 33 | T2A | GAGGGCAGAGGAAGTCTTCTAACATGCGGTGACGTGGAGGAGA |
| | | ATCCTGGCCCG |
| 34 | P2A | GGAAGCGGAGCTACTAACTTCAGCCTGCTGAAGCAGGCTGGAG |
| | | ACGTGGAGGAGAACCCTGGACCT |
| 35 | E2A | CAGTGTACTAATTATGCTCTCTTGAAATTGGCTGGAGATGTTG |
| | | AGAGCAACCCTGGACCT |
| 36 | F2A | GTGAAACAGACTTTGAATTTTGACCTTCTCAAGTTGGCGGGAG |
| | | ACGTGGAGTCCAACCCTGGACCT |
| 37 | IRES | CCCCTCTCCCTCCCCCCCCCCTAACGTTACTGGCCGAAGCCGC |
| | | TTGGAATAAGGCCGGTGTGCGTTTGTCTATATGTTATTTTCCA |
| | | CCATATTGCCGTCTTTTGGCAATGTGAGGGCCCGGAAACCTGG |
| | | CCCTGTCTTCTTGACGAGCATTCCTAGGGGTCTTTCCCCTCTC |
| | | GCCAAAGGAATGCAAGGTCTGTTGAATGTCGTGAAGGAAGCAG |
| | | TTCCTCTGGAAGCTTCTTGAAGACAAACAACGTCTGTAGCGAC |
| | | CCTTTGCAGGCAGCGGAACCCCCCACCTGGCGACAGGTGCCTC |
| | | TGCGGCCAAAAGCCACGTGTATAAGATACACCTGCAAAGGCGG |
| | | CACAACCCCAGTGCCACGTTGTGAGTTGGATAGTTGTGGAAAG |
| | | AGTCAAATGGCTCTCCTCAAGCGTATTCAACAAGGGGCTGAAG |
| | | GATGCCCAGAAGGTACCCCATTGTATGGGATCTGATCTGGGGC |
| | | CTCGGTGCACATGCTTTACATGTGTTTAGTCGAGGTTAAAAAA |
| | | ACGTCTAGGCCCCCCGAACCACGGGGACGTGGTTTTCCTTTGA |
| | | AAAACACGATGATAA |

### Essential Genes

An essential gene can be any gene that is essential for the survival and/or the proliferation of the cell. In some embodiments, an essential gene is a housekeeping gene that is essential for survival of all cell types, e.g., a gene listed in Table 3. See also other housekeeping genes discussed in Eisenberg, Trends in Gen. 2014; 30(3):119-20 and Moein et al., Adv. Biomed Res. 2017; 6:15. Additional genes that are essential for various cell types, including iPSCs/ESCs, are listed in Table 4 (see also the essential genes discussed in Yilmaz et al., Nat. Cell Biol. 2018; 20:610-619 the entire contents of which are incorporated herein by reference).

In some embodiments the essential gene is GAPDH and the DNA nuclease causes a break in exon 9, e.g., a double-strand break. In some embodiments the essential gene is TBP and the DNA nuclease causes a break in exon 7, or exon 8, e.g., a double-strand break. In some embodiments the essential gene is E2F4 and the DNA nuclease causes a break in exon 10, e.g., a double-strand break. In some embodiments the essential gene is G6PD and the DNA nuclease causes a break in exon 13, e.g., a double-strand break. In some embodiments the essential gene is KIF11 and the DNA nuclease causes a break in exon 22, e.g., a double-strand break.

TABLE 3
Exemplary housekeeping genes

| Ensembl ID | Gene Symbol |
|---|---|
| ENSG00000075624 | ACTB |
| ENSG00000116459 | ATP5F1 |
| ENSG00000166710 | B2M |
| ENSG00000111640 | GAPDH |
| ENSG00000169919 | GUSB |
| ENSG00000165704 | HPRT1 |
| ENSG00000102144 | PGK1 |
| ENSG00000196262 | PPIA |
| ENSG00000138160 | KIF11 |
| ENSG00000231500 | RPS18 |
| ENSG00000112592 | TBP |
| ENSG00000072274 | TFRC |
| ENSG00000164924 | YWHAZ |
| ENSG00000089157 | RPLP0 |
| ENSG00000142541 | RPL13A |
| ENSG00000147604 | RPL7 |
| ENSG00000205250 | E2F4 |
| ENSG00000160211 | G6PD |

TABLE 4
Additional exemplary essential genes

| Ensembl ID | Gene Symbol |
|---|---|
| ENSG00000111704 | NANOG |
| ENSG00000179059 | ZFP42 |
| ENSG00000136826 | KLF4 |
| ENSG00000118655 | DCLRE1B |
| ENSG00000172409 | CLP1 |
| ENSG00000082898 | XPO1 |
| ENSG00000114867 | EIF4G1 |
| ENSG00000115866 | DARS |
| ENSG00000204628 | GNB2L1 |
| ENSG00000198242 | RPL23A |
| ENSG00000158526 | TSR2 |
| ENSG00000125450 | NUP85 |
| ENSG00000134371 | CDC73 |
| ENSG00000164941 | INTS8 |
| ENSG00000055783 | USP36 |
| ENSG00000258366 | RTEL1 |
| ENSG00000188846 | RPL14 |
| ENSG00000247626 | MARS2 |
| ENSG00000095787 | WAC |
| ENSG00000108094 | CUL2 |
| ENSG00000185946 | RNPC3 |
| ENSG00000154473 | BUB3 |
| ENSG00000204394 | VARS |
| ENSG00000103051 | COG4 |
| ENSG00000104738 | MCM4 |
| ENSG00000117222 | RBBP5 |
| ENSG00000082516 | GEMIN5 |
| ENSG00000100162 | CENPM |
| ENSG00000141456 | PELP1 |
| ENSG00000137807 | KIF23 |
| ENSG00000112685 | EXOC2 |
| ENSG00000125995 | ROMO1 |
| ENSG00000136891 | TEX10 |
| ENSG00000173113 | TRMT112 |
| ENSG00000075914 | EXOSC7 |
| ENSG00000119523 | ALG2 |
| ENSG00000244038 | DDOST |
| ENSG00000108175 | ZMIZ1 |
| ENSG00000129691 | ASH2L |
| ENSG00000183207 | RUVBL2 |
| ENSG00000055044 | NOP58 |
| ENSG00000204315 | FKBPL |
| ENSG00000187522 | HSPA14 |
| ENSG00000169375 | SIN3A |
| ENSG00000143748 | NVL |
| ENSG00000021776 | AQR |
| ENSG00000132467 | UTP3 |
| ENSG00000087470 | DNM1L |
| ENSG00000130811 | EIF3G |
| ENSG00000180198 | RCC1 |
| ENSG00000101407 | TTI1 |
| ENSG00000116455 | WDR77 |
| ENSG00000135763 | URB2 |
| ENSG00000133316 | WDR74 |
| ENSG00000189091 | SF3B3 |
| ENSG00000109917 | ZNF259 |
| ENSG00000130640 | TUBGCP2 |
| ENSG00000011376 | LARS2 |
| ENSG00000135249 | RINT1 |
| ENSG00000126883 | NUP214 |
| ENSG00000163510 | CWC22 |
| ENSG00000101138 | CSTF1 |
| ENSG00000104221 | BRF2 |
| ENSG00000125630 | POLR1B |
| ENSG00000083896 | YTHDC1 |
| ENSG00000105726 | ATP13A1 |
| ENSG00000105618 | PRPF31 |
| ENSG00000117748 | RPA2 |
| ENSG00000143294 | PRCC |
| ENSG00000156239 | N6AMT1 |
| ENSG00000143384 | MCL1 |
| ENSG00000113407 | TARS |
| ENSG00000086589 | RBM22 |
| ENSG00000133119 | RFC3 |
| ENSG00000052749 | RRP12 |
| ENSG00000103047 | TANGO6 |
| ENSG00000142751 | GPN2 |
| ENSG00000101057 | MYBL2 |
| ENSG00000176915 | ANKLE2 |
| ENSG00000071127 | WDR1 |
| ENSG00000106344 | RBM28 |
| ENSG00000100316 | RPL3 |
| ENSG00000139131 | YARS2 |
| ENSG00000182831 | C16orf72 |
| ENSG00000167325 | RRM1 |
| ENSG00000172262 | ZNF131 |
| ENSG00000007168 | PAFAH1B1 |
| ENSG00000117174 | ZNHIT6 |
| ENSG00000196497 | IPO4 |
| ENSG00000188566 | NDOR1 |
| ENSG00000183091 | NEB |
| ENSG00000011304 | PTBP1 |
| ENSG00000109805 | NCAPG |
| ENSG00000123154 | WDR83 |
| ENSG00000147416 | ATP6V1B2 |
| ENSG00000163961 | RNF168 |
| ENSG00000163811 | WDR43 |
| ENSG00000143624 | INTS3 |
| ENSG00000101161 | PRPF6 |
| ENSG00000130726 | TRIM28 |
| ENSG00000165494 | PCF11 |
| ENSG00000053900 | ANAPC4 |
| ENSG00000168255 | POLR2J3 |
| ENSG00000129534 | MIS18BP1 |
| ENSG00000164754 | RAD21 |
| ENSG00000120158 | RCL1 |
| ENSG00000161016 | RPL8 |
| ENSG00000030066 | NUP160 |
| ENSG00000099624 | ATP5D |
| ENSG00000116120 | FARSB |
| ENSG00000115233 | PSMD14 |
| ENSG00000086504 | MRPL28 |
| ENSG00000160752 | FDPS |
| ENSG00000049541 | RFC2 |
| ENSG00000148688 | RPP30 |
| ENSG00000114573 | ATP6V1A |
| ENSG00000086200 | IPO11 |
| ENSG00000119720 | NRDE2 |
| ENSG00000058262 | SEC61A1 |
| ENSG00000073111 | MCM2 |
| ENSG00000138160 | KIF11 |
| ENSG00000215193 | PEX26 |
| ENSG00000161057 | PSMC2 |
| ENSG00000187514 | PTMA |
| ENSG00000135829 | DHX9 |
| ENSG00000058729 | RIOK2 |
| ENSG00000110330 | BIRC2 |
| ENSG00000141759 | TXNL4A |
| ENSG00000166986 | MARS |
| ENSG00000153774 | CFDP1 |
| ENSG00000130177 | CDC16 |
| ENSG00000241553 | ARPC4 |
| ENSG00000132604 | TERF2 |
| ENSG00000114982 | KANSL3 |
| ENSG00000213780 | GTF2H4 |
| ENSG00000139343 | SNRPF |
| ENSG00000101189 | MRGBP |
| ENSG00000079246 | XRCC5 |
| ENSG00000196943 | NOP9 |
| ENSG00000122965 | RBM19 |
| ENSG00000132383 | RPA1 |
| ENSG00000094880 | CDC23 |
| ENSG00000213639 | PPP1CB |
| ENSG00000109911 | ELP4 |
| ENSG00000180957 | PITPNB |
| ENSG00000122257 | RBBP6 |
| ENSG00000173145 | NOC3L |
| ENSG00000179115 | FARSA |
| ENSG00000105171 | POP4 |
| ENSG00000148303 | RPL7A |
| ENSG00000167508 | MVD |
| ENSG00000115541 | HSPE1 |
| ENSG00000170445 | HARS |
| ENSG00000168496 | FEN1 |
| ENSG00000141367 | CLTC |
| ENSG00000087191 | PSMC5 |
| ENSG00000163159 | VPS72 |
| ENSG00000130741 | EIF2S3 |
| ENSG00000168495 | POLR3D |
| ENSG00000071894 | CPSF1 |
| ENSG00000058600 | POLR3E |
| ENSG00000100726 | TELO2 |
| ENSG00000165501 | LRR1 |
| ENSG00000113575 | PPP2CA |
| ENSG00000116922 | C1orf109 |
| ENSG00000073712 | FERMT2 |
| ENSG00000174437 | ATP2A2 |
| ENSG00000176407 | KCMF1 |
| ENSG00000140525 | FANCI |
| ENSG00000101182 | PSMA7 |
| ENSG00000130204 | TOMM40 |
| ENSG00000239306 | RBM14 |
| ENSG00000248643 | RBM14-RBM4 |
| ENSG00000172113 | NME6 |
| ENSG00000136448 | NMT1 |
| ENSG00000186166 | CCDC84 |
| ENSG00000166233 | ARIH1 |
| ENSG00000111877 | MCM9 |
| ENSG00000204316 | MRPL38 |
| ENSG00000101868 | POLA1 |
| ENSG00000107951 | MTPAP |
| ENSG00000039650 | PNKP |
| ENSG00000123064 | DDX54 |
| ENSG00000183955 | SETD8 |
| ENSG00000138107 | ACTR1A |
| ENSG00000244005 | NFS1 |
| ENSG00000188986 | NELFB |
| ENSG00000018699 | TTC27 |
| ENSG00000167112 | TRUB2 |
| ENSG00000100393 | EP300 |
| ENSG00000101639 | CEP192 |
| ENSG00000126461 | SCAF1 |
| ENSG00000172171 | TEFM |
| ENSG00000135913 | USP37 |
| ENSG00000135624 | CCT7 |
| ENSG00000100804 | PSMB5 |
| ENSG00000175792 | RUVBL1 |
| ENSG00000183431 | SF3A3 |
| ENSG00000108773 | KAT2A |
| ENSG00000100949 | RABGGTA |
| ENSG00000151503 | NCAPD3 |
| ENSG00000111880 | RNGTT |
| ENSG00000168883 | USP39 |
| ENSG00000151461 | UPF2 |
| ENSG00000105486 | LIG1 |
| ENSG00000111300 | NAA25 |
| ENSG00000144559 | TAMM41 |
| ENSG00000137574 | TGS1 |
| ENSG00000172273 | HINFP |
| ENSG00000133112 | TPT1 |
| ENSG00000167986 | DDB1 |
| ENSG00000125319 | C17orf53 |
| ENSG00000113161 | HMGCR |
| ENSG00000100941 | PNN |
| ENSG00000139697 | SBNO1 |
| ENSG00000135336 | ORC3 |
| ENSG00000101115 | SALL4 |
| ENSG00000100902 | PSMA6 |
| ENSG00000141141 | DDX52 |
| ENSG00000254093 | PINX1 |
| ENSG00000184445 | KNTC1 |
| ENSG00000089053 | ANAPC5 |
| ENSG00000111602 | TIMELESS |
| ENSG00000145592 | RPL37 |
| ENSG00000106615 | RHEB |
| ENSG00000180817 | PPA1 |
| ENSG00000110172 | CHRODC1 |
| ENSG00000137876 | RSL24D1 |
| ENSG00000104408 | EIF3E |
| ENSG00000143436 | MRPL9 |
| ENSG00000108883 | EFTUD2 |
| ENSG00000140740 | UQCRC2 |
| ENSG00000211456 | SACM1L |
| ENSG00000131051 | RBM39 |
| ENSG00000136758 | YME1L1 |
| ENSG00000112578 | BYSL |
| ENSG00000163781 | TOPBP1 |
| ENSG00000106628 | POLD2 |
| ENSG00000132952 | USPL1 |
| ENSG00000168538 | TRAPPC11 |
| ENSG00000168488 | ATXN2L |
| ENSG00000022277 | RTFDC1 |
| ENSG00000179988 | PSTK |
| ENSG00000092199 | HNRNPC |
| ENSG00000156831 | NSMCE2 |
| ENSG00000125691 | RPL23 |
| ENSG00000083520 | DIS3 |
| ENSG00000115761 | NOL10 |
| ENSG00000173894 | CBX2 |
| ENSG00000243147 | MRPL33 |
| ENSG00000139618 | BRCA2 |
| ENSG00000109519 | GRPEL1 |
| ENSG00000203760 | CENPW |
| ENSG00000166851 | PLK1 |
| ENSG00000121579 | NAA50 |
| ENSG00000163608 | C3orf17 |
| ENSG00000005075 | POLR2J |
| ENSG00000148606 | POLR3A |
| ENSG00000160949 | TONSL |
| ENSG00000128159 | TUBGCP6 |
| ENSG00000125449 | ARMC7 |
| ENSG00000122406 | RPL5 |
| ENSG00000126226 | PCID2 |
| ENSG00000159377 | PSMB4 |
| ENSG00000167967 | E4F1 |
| ENSG00000141076 | CIRH1A |
| ENSG00000069248 | NUP133 |
| ENSG00000242372 | EIF6 |
| ENSG00000087269 | NOP14 |
| ENSG00000163468 | CCT3 |
| ENSG00000140326 | CDAN1 |
| ENSG00000146834 | MEPCE |
| ENSG00000143222 | UFC1 |
| ENSG00000110871 | COQ5 |
| ENSG00000119285 | HEATR1 |
| ENSG00000145386 | CCNA2 |
| ENSG00000164109 | MAD2L1 |
| ENSG00000185347 | C14orf80 |
| ENSG00000134748 | PRPF38A |
| ENSG00000070061 | IKBKAP |
| ENSG00000099995 | SF3A1 |
| ENSG00000100029 | PES1 |
| ENSG00000130255 | RPL36 |
| ENSG00000085231 | AK6 |
| ENSG00000187145 | MRPS21 |
| ENSG00000062650 | WAPAL |
| ENSG00000122484 | RPAP2 |
| ENSG00000090861 | AARS |
| ENSG00000161888 | SPC24 |
| ENSG00000087087 | SRRT |
| ENSG00000134910 | STT3A |
| ENSG00000161526 | SAP30BP |
| ENSG00000068654 | POLR1A |
| ENSG00000140983 | RHOT2 |
| ENSG00000184708 | EIF4ENIF1 |
| ENSG00000100479 | POLE2 |
| ENSG00000134440 | NARS |
| ENSG00000014164 | ZC3H3 |
| ENSG00000113812 | ACTR8 |
| ENSG00000145331 | TRMT10A |
| ENSG00000110104 | CCDC86 |
| ENSG00000164163 | ABCE1 |
| ENSG00000167863 | ATP5H |
| ENSG00000176946 | THAP4 |
| ENSG00000169251 | NMD3 |
| ENSG00000166226 | CCT2 |
| ENSG00000131747 | TOP2A |
| ENSG00000267673 | FDX1L |
| ENSG00000108559 | NUP88 |
| ENSG00000104957 | CCDC130 |
| ENSG00000167522 | ANKRD11 |
| ENSG00000130706 | ADRM1 |
| ENSG00000048162 | NOP16 |
| ENSG00000159210 | SNF8 |
| ENSG00000113360 | DROSHA |
| ENSG00000108296 | CWC25 |
| ENSG00000161395 | PGAP3 |
| ENSG00000089195 | TRMT6 |
| ENSG00000185838 | GNB1L |
| ENSG00000101146 | RAE1 |
| ENSG00000092853 | CLSPN |
| ENSG00000107949 | BCCIP |
| ENSG00000159079 | C21orf59 |
| ENSG00000137947 | GTF2B |
| ENSG00000160948 | VPS28 |
| ENSG00000065427 | KARS |
| ENSG00000102978 | POLR2C |
| ENSG00000182154 | MRPL41 |
| ENSG00000139168 | ZCRB1 |
| ENSG00000175110 | MRPS22 |
| ENSG00000177084 | POLE |
| ENSG00000197681 | TBC1D3 |
| ENSG00000053501 | USE1 |
| ENSG00000121879 | PIK3CA |
| ENSG00000108278 | ZNHIT3 |
| ENSG00000161547 | SRSF2 |
| ENSG00000129083 | COPB1 |
| ENSG00000012048 | BRCA1 |
| ENSG00000171314 | PGAM1 |
| ENSG00000112159 | MDN1 |
| ENSG00000174243 | DDX23 |
| ENSG00000096401 | CDC5L |
| ENSG00000128513 | POT1 |
| ENSG00000071859 | FAM50A |
| ENSG00000100084 | HIRA |
| ENSG00000100813 | ACIN1 |
| ENSG00000005100 | DHX33 |
| ENSG00000101158 | NELFCD |
| ENSG00000115946 | PNO1 |
| ENSG00000188647 | PTAR1 |
| ENSG00000146007 | ZMAT2 |
| ENSG00000241837 | ATP5O |
| ENSG00000113643 | RARS |
| ENSG00000162521 | RBBP4 |
| ENSG00000116830 | TTF2 |
| ENSG00000187555 | USP7 |
| ENSG00000137216 | TMEM63B |
| ENSG00000161904 | LEMD62 |
| ENSG00000241945 | PWP2 |
| ENSG00000134982 | APC |
| ENSG00000156983 | BRPF1 |
| ENSG00000164346 | NSA2 |
| ENSG00000223496 | EXOSC6 |
| ENSG00000113569 | NUP155 |
| ENSG00000080986 | NDC80 |
| ENSG00000143374 | TARS2 |
| ENSG00000104835 | SARS2 |
| ENSG00000152253 | SPC25 |
| ENSG00000088356 | PDRG1 |
| ENSG00000044574 | HSPA5 |
| ENSG00000116874 | WARS2 |
| ENSG00000204531 | POU5F1 |
| ENSG00000004779 | NDUFAB1 |
| ENSG00000161981 | SNRNP25 |
| ENSG00000126457 | PRMT1 |
| ENSG00000142507 | PSMB6 |
| ENSG00000164808 | SPIDR |
| ENSG00000234972 | TBC1D3C |
| ENSG00000144554 | FANCD2 |
| ENSG00000147383 | NSDHL |
| ENSG00000165732 | DDX21 |
| ENSG00000155975 | VPS37A |
| ENSG00000002822 | MAD1L1 |
| ENSG00000179271 | GADD45GIP1 |
| ENSG00000101452 | DHX35 |
| ENSG00000074071 | MRPS34 |
| ENSG00000169045 | HNRNPH1 |
| ENSG00000087510 | TFAP2C |
| ENSG00000105819 | PMPCB |
| ENSG00000204351 | SKIV2L |
| ENSG00000160783 | PMF1 |
| ENSG00000152234 | ATP5A1 |
| ENSG00000127463 | EMC1 |
| ENSG00000124228 | DDX27 |
| ENSG00000100319 | ZMAT5 |
| ENSG00000065183 | WDR3 |
| ENSG00000058272 | PPP1R12A |
| ENSG00000136628 | EPRS |
| ENSG00000163017 | ACTG2 |
| ENSG00000104884 | ERCC2 |
| ENSG00000166483 | WEE1 |
| ENSG00000135837 | CEP350 |
| ENSG00000104897 | SF3A2 |
| ENSG00000140598 | EFTUD1 |
| ENSG00000143774 | GUK1 |
| ENSG00000085721 | RRN3 |
| ENSG00000172053 | QARS |
| ENSG00000165934 | CPSF2 |
| ENSG00000052802 | MSMO1 |
| ENSG00000135476 | ESPL1 |
| ENSG00000174177 | CTU2 |
| ENSG00000120438 | TCP1 |
| ENSG00000170892 | TSEN34 |
| ENSG00000204574 | ABCF1 |
| ENSG00000175376 | EIF1AD |
| ENSG00000146263 | MMS22L |
| ENSG00000121022 | COPS5 |
| ENSG00000168090 | COPS6 |
| ENSG00000167491 | GATAD2A |
| ENSG00000084072 | PPIE |
| ENSG00000115268 | RPS15 |
| ENSG00000163938 | GNL3 |
| ENSG00000151665 | PIGF |
| ENSG00000148843 | PDCD11 |
| ENSG00000141736 | ERBB2 |
| ENSG00000103168 | TAF1C |
| ENSG00000105401 | CDC37 |
| ENSG00000163933 | RFT1 |
| ENSG00000122085 | MTERFD2 |
| ENSG00000164032 | H2AFZ |
| ENSG00000140943 | MBTPS1 |
| ENSG00000198952 | SMG5 |
| ENSG00000169021 | UQCRFS1 |
| ENSG00000013810 | TACC3 |
| ENSG00000105258 | POLR2I |
| ENSG00000167978 | SRRM2 |
| ENSG00000095564 | BTAF1 |
| ENSG00000138095 | LRPPRC |
| ENSG00000063978 | RNF4 |
| ENSG00000162368 | CMPK1 |
| ENSG00000140829 | DHX38 |
| ENSG00000158169 | FANCC |
| ENSG00000161960 | EIF4A1 |
| ENSG00000181222 | POLR2A |
| ENSG00000165916 | PSMC3 |
| ENSG00000198060 | MARCH5 |
| ENSG00000149923 | PPP4C |
| ENSG00000111667 | USP5 |
| ENSG00000198755 | RPL10A |
| ENSG00000141499 | WRAP53 |
| ENSG00000093009 | CDC45 |
| ENSG00000105732 | ZNF574 |
| ENSG00000104064 | GABPB1 |
| ENSG00000108294 | PSMB3 |
| ENSG00000130856 | NZF236 |
| ENSG00000133980 | VRTN |
| ENSG00000149308 | NPAT |
| ENSG00000120071 | KANSL1 |
| ENSG00000129084 | PSMA1 |
| ENSG00000117877 | CD3EAP |
| ENSG00000127616 | SMARCA4 |
| ENSG00000163882 | POLR2H |
| ENSG00000183718 | TRIM52 |
| ENSG00000106803 | SEC61B |
| ENSG00000114942 | EEF1B2 |
| ENSG00000067704 | IARS2 |
| ENSG00000114686 | MRPL3 |
| ENSG00000172315 | TP53RK |
| ENSG00000173120 | KDM2A |
| ENSG00000138442 | WDR12 |
| ENSG00000145982 | FARS2 |
| ENSG00000117481 | NSUN4 |
| ENSG00000142676 | RPL11 |
| ENSG00000164615 | CAMLG |
| ENSG00000138073 | PREB |
| ENSG00000136888 | ATP6V1G1 |
| ENSG00000221829 | FANCG |
| ENSG00000198887 | SMC5 |
| ENSG00000102900 | NUP93 |
| ENSG00000108344 | PSMD3 |
| ENSG00000023191 | RNH1 |
| ENSG00000143621 | ILF2 |
| ENSG00000112855 | HARS2 |
| ENSG00000110536 | PTPMT1 |
| ENSG00000165629 | ATP5C1 |
| ENSG00000166847 | DCTN5 |
| ENSG00000104852 | SNRNP70 |
| ENSG00000203814 | HIST2H2BF |
| ENSG00000009413 | REV3L |
| ENSG00000130772 | MED18 |
| ENSG00000079313 | REXO1 |
| ENSG00000012061 | ERCC1 |
| ENSG00000111642 | CHD4 |
| ENSG00000100462 | PRMT5 |
| ENSG00000174100 | MRPL45 |
| ENSG00000101421 | CHMP4B |
| ENSG00000144028 | SNRNP200 |
| ENSG00000108592 | FTSJ3 |
| ENSG00000110048 | OSBP |
| ENSG00000147403 | RPL10 |
| ENSG00000198783 | ZNF830 |
| ENSG00000179409 | GEMIN4 |
| ENSG00000147604 | RPL7 |
| ENSG00000136824 | SMC2 |
| ENSG00000104889 | RNASEH2A |
| ENSG00000146282 | RARS2 |
| ENSG00000068784 | SRBD1 |
| ENSG00000137822 | TUBGCP4 |
| ENSG00000059691 | PET112 |
| ENSG00000066827 | ZFAT |
| ENSG00000148308 | GTF3C5 |
| ENSG00000170185 | USP38 |
| ENSG00000160201 | U2AF1 |
| ENSG00000141258 | SGSM2 |
| ENSG00000172660 | TAF15 |
| ENSG00000145833 | DDX46 |
| ENSG00000104980 | TIMM44 |
| ENSG00000097046 | CDC7 |
| ENSG00000131368 | MRPS25 |
| ENSG00000204209 | DAXX |
| ENSG00000129696 | TTI2 |
| ENSG00000108848 | LUC7L3 |
| ENSG00000013573 | DDX11 |
| ENSG00000105248 | CCDC94 |
| ENSG00000183598 | HIST2H3D |
| ENSG00000224226 | TBC1D3B |
| ENSG00000090470 | PDCD7 |
| ENSG00000031698 | SARS |
| ENSG00000108270 | AATF |
| ENSG00000159111 | MRPL10 |
| ENSG00000149806 | FAU |
| ENSG00000188739 | RBM34 |
| ENSG00000152684 | PELO |
| ENSG00000174374 | WBSCR16 |
| ENSG00000107036 | KIAA1432 |
| ENSG00000204619 | PPP1R11 |
| ENSG00000091651 | ORC6 |
| ENSG00000134480 | CCNH |
| ENSG00000164151 | KIAA0947 |
| ENSG00000164611 | PTTG1 |
| ENSG00000111445 | RFC5 |
| ENSG00000127481 | UBR4 |
| ENSG00000159352 | PSMD4 |
| ENSG00000137814 | HAUS2 |
| ENSG00000105220 | GPI |
| ENSG00000140521 | POLG |
| ENSG00000075856 | SART3 |
| ENSG00000143742 | SRP9 |
| ENSG00000163029 | SMC6 |
| ENSG00000162227 | TAF6L |
| ENSG00000100129 | EIF3L |
| ENSG00000170348 | TMED10 |
| ENSG00000182217 | HIST2H4B |
| ENSG00000183941 | HIST2H4A |
| ENSG00000116221 | MRPL37 |
| ENSG00000196235 | SUPT5H |
| ENSG00000161920 | MED11 |
| ENSG00000134690 | CDCA8 |
| ENSG00000131153 | GINS2 |
| ENSG00000138018 | EPT1 |
| ENSG00000173141 | MRP63 |
| ENSG00000154727 | GABPA |
| ENSG00000120800 | UTP20 |
| ENSG00000114767 | RRP9 |
| ENSG00000174231 | PRPF8 |
| ENSG00000137547 | MRPL15 |
| ENSG00000146576 | C7orf26 |
| ENSG00000065268 | WDR18 |
| ENSG00000147162 | OGT |
| ENSG00000198917 | C9orf114 |
| ENSG00000180822 | PSMG4 |
| ENSG00000125977 | EIF2S2 |
| ENSG00000173418 | NAA20 |
| ENSG00000155561 | NUP205 |
| ENSG00000173545 | ZNF622 |
| ENSG00000127993 | RBM48 |
| ENSG00000197102 | DYNC1H1 |
| ENSG00000119392 | GLE1 |
| ENSG00000174444 | RPL4 |
| ENSG00000149716 | ORAOV1 |
| ENSG00000155876 | RRAGA |
| ENSG00000198841 | KTI12 |
| ENSG00000056097 | ZFR |
| ENSG00000227057 | WDR46 |
| ENSG00000167670 | CHAF1A |
| ENSG00000127191 | TRAF2 |
| ENSG00000072506 | HSD17B10 |
| ENSG00000215021 | PHB2 |
| ENSG00000175467 | SART1 |
| ENSG00000121073 | SLC35B1 |
| ENSG00000079459 | FDFT1 |
| ENSG00000143493 | INTS7 |
| ENSG00000141543 | EIF4A3 |
| ENSG00000174197 | MGA |
| ENSG00000131269 | ABCB7 |
| ENSG00000089009 | RPL6 |
| ENSG00000197780 | TAF13 |
| ENSG00000036549 | ZZZ3 |
| ENSG00000066135 | KDM4A |
| ENSG00000176473 | WDR25 |
| ENSG00000124614 | RPS10 |
| ENSG00000107581 | EIF3A |
| ENSG00000084463 | WBP11 |
| ENSG00000137656 | BUD13 |
| ENSG00000183751 | TBL3 |
| ENSG00000119537 | KDSR |
| ENSG00000204220 | PFDN6 |
| ENSG00000170291 | ELP5 |
| ENSG00000198563 | DDX39B |
| ENSG00000077549 | CAPZB |
| ENSG00000255529 | POLR2M |
| ENSG00000100034 | PPM1F |
| ENSG00000196367 | TRRAP |
| ENSG00000167258 | CDK12 |
| ENSG00000039123 | SKIV2L2 |
| ENSG00000076043 | REXO2 |
| ENSG00000213676 | ATF6B |
| ENSG00000058453 | CROCC |
| ENSG00000153575 | TUBGCP5 |
| ENSG00000110700 | RPS13 |
| ENSG00000101181 | MTG2 |
| ENSG00000071539 | TRIP13 |
| ENSG00000075702 | WDR62 |
| ENSG00000171453 | POLR1C |
| ENSG00000090989 | EXOC1 |
| ENSG00000037897 | METTL1 |
| ENSG00000095139 | ARCN1 |
| ENSG00000078142 | PIK3C3 |
| ENSG00000141030 | COPS3 |
| ENSG00000126249 | PDCD2L |
| ENSG00000117408 | IPO13 |
| ENSG00000130725 | UBE2M |
| ENSG00000175054 | ATR |
| ENSG00000149016 | TUT1 |
| ENSG00000165060 | FXN |
| ENSG00000117597 | DIEXF |
| ENSG00000185085 | INTS5 |
| ENSG00000113595 | TRIM23 |
| ENSG00000040633 | PHF23 |
| ENSG00000178952 | TUFM |
| ENSG00000120539 | MASTL |
| ENSG00000103549 | RNF40 |
| ENSG00000119723 | COQ6 |
| ENSG00000171311 | EXOSC1 |
| ENSG00000106245 | BUD31 |
| ENSG00000118046 | STK11 |
| ENSG00000125484 | GTF3C4 |
| ENSG00000089094 | KDM2B |
| ENSG00000121621 | KIF18A |
| ENSG00000129911 | KLF16 |
| ENSG00000102302 | FGD1 |
| ENSG00000135679 | MDM2 |
| ENSG00000185115 | NDNL2 |
| ENSG00000140553 | UNC45A |
| ENSG00000129562 | DAD1 |
| ENSG00000100138 | NHP2L1 |
| ENSG00000111641 | NOP2 |
| ENSG00000173660 | UQCRH |
| ENSG00000198677 | TTC37 |
| ENSG00000135503 | ACVR1B |
| ENSG00000180998 | GPR137C |
| ENSG00000153187 | HNRNPU |
| ENSG00000106459 | NRF1 |
| ENSG00000156261 | CCT8 |
| ENSG00000118363 | SPCS2 |
| ENSG00000164134 | NAA15 |
| ENSG00000060642 | PIGV |
| ENSG00000090889 | KIF4A |
| ENSG00000101361 | NOP56 |
| ENSG00000167792 | NDUFV1 |
| ENSG00000184162 | NR2C2AP |
| ENSG00000128524 | ATP6V1F |
| ENSG00000100387 | RBX1 |
| ENSG00000110906 | KCTD10 |
| ENSG00000147457 | CHMP7 |
| ENSG00000124570 | SERPINB6 |
| ENSG00000186468 | RPS23 |
| ENSG00000136122 | BORA |
| ENSG00000047249 | ATP6V1H |
| ENSG00000127804 | METTL16 |
| ENSG00000104412 | EMC2 |
| ENSG00000173726 | TOMM20 |
| ENSG00000138777 | PPA2 |
| ENSG00000170043 | TRAPPC11 |
| ENSG00000168488 | ATXN2L |
| ENSG00000022277 | RTFDC1 |
| ENSG00000179988 | PSTK |
| ENSG00000092199 | HNRNPC |
| ENSG00000156831 | NSMCE2 |
| ENSG00000125691 | RPL23 |
| ENSG00000083520 | DIS3 |
| ENSG00000115761 | NOL10 |
| ENSG00000173894 | CBX2 |
| ENSG00000243147 | MRPL33 |
| ENSG00000139618 | BRCA2 |
| ENSG00000109519 | GRPEL1 |
| ENSG00000203760 | CENPW |
| ENSG00000166851 | PLK1 |
| ENSG00000121579 | NAA50 |
| ENSG00000163608 | C3orf17 |
| ENSG00000005075 | POLR2J |
| ENSG00000148606 | POLR3A |
| ENSG00000160949 | TONSL |
| ENSG00000128159 | TUBGCP6 |
| ENSG00000125449 | ARMC7 |
| ENSG00000122406 | RPL5 |
| ENSG00000126226 | PCID2 |
| ENSG00000159377 | PSMB4 |
| ENSG00000167967 | E4F1 |
| ENSG00000141076 | CIRH1A |
| ENSG00000069248 | NUP133 |
| ENSG00000242372 | EIF6 |
| ENSG00000087269 | NOP14 |
| ENSG00000163468 | CCT3 |
| ENSG00000140326 | CDAN1 |
| ENSG00000146834 | MEPCE |
| ENSG00000143222 | UFC1 |
| ENSG00000110871 | COQ5 |
| ENSG00000119285 | HEATR1 |
| ENSG00000145386 | CCNA2 |
| ENSG00000164109 | MAD2L1 |
| ENSG00000185347 | C14orf80 |
| ENSG00000134748 | PRPF38A |
| ENSG00000070061 | IKBKAP |
| ENSG00000099995 | SF3A1 |
| ENSG00000100029 | PES1 |
| ENSG00000130255 | RPL36 |
| ENSG00000085231 | AK6 |
| ENSG00000187145 | MRPS21 |
| ENSG00000062650 | WAPAL |
| ENSG00000122484 | RPAP2 |
| ENSG00000090861 | AARS |
| ENSG00000161888 | SPC24 |
| ENSG00000087087 | SRRT |
| ENSG00000134910 | STT3A |
| ENSG00000161526 | SAP30BP |
| ENSG00000068654 | POLR1A |
| ENSG00000140983 | RHOT2 |
| ENSG00000184708 | EIF4ENIF1 |
| ENSG00000100479 | POLE2 |
| ENSG00000134440 | NARS |
| ENSG00000014164 | ZC3H3 |
| ENSG00000113812 | ACTR8 |
| ENSG00000145331 | TRMT10A |
| ENSG00000110104 | CCDC86 |
| ENSG00000164163 | ABCE1 |
| ENSG00000167863 | ATP5H |
| ENSG00000176946 | THAP4 |
| ENSG00000169251 | NMD3 |
| ENSG00000166226 | CCT2 |
| ENSG00000131747 | TOP2A |
| ENSG00000267673 | TDX1L |
| ENSG00000108559 | NUP88 |
| ENSG00000104957 | CCDC130 |
| ENSG00000167522 | ANKRD11 |
| ENSG00000130706 | ADRM1 |
| ENSG00000048162 | NOP16 |
| ENSG00000159210 | SNF8 |
| ENSG00000113360 | DROSHA |
| ENSG00000108296 | CWC25 |
| ENSG00000161395 | PGAP3 |
| ENSG00000089195 | TRMT6 |
| ENSG00000185838 | GNB1L |
| ENSG00000101146 | RAE1 |
| ENSG00000092853 | CLSPN |
| ENSG00000107949 | BCCIP |
| ENSG00000159079 | C21orf59 |
| ENSG00000137947 | GTF2B |
| ENSG00000160948 | VPS28 |
| ENSG00000065427 | KARS |
| ENSG00000102978 | POLR2C |
| ENSG00000182154 | MRPL41 |
| ENSG00000139168 | ZCRB1 |
| ENSG00000175110 | MRPS22 |
| ENSG00000177084 | POLE |
| ENSG00000197681 | TBC1D3 |
| ENSG00000053501 | USE1 |
| ENSG00000121879 | PIK3CA |
| ENSG00000108278 | ZNHIT3 |
| ENSG00000161547 | SRSF2 |
| ENSG00000129083 | COPB1 |
| ENSG00000012048 | BRCA1 |
| ENSG00000171314 | PGAM1 |
| ENSG00000112159 | MDN1 |
| ENSG00000174243 | DDX23 |
| ENSG00000096401 | CDC5L |
| ENSG00000128513 | POT1 |
| ENSG00000071859 | FAM50A |
| ENSG00000100084 | HIRA |
| ENSG00000100813 | ACIN1 |
| ENSG00000005100 | DHX33 |
| ENSG00000101158 | NELFCD |
| ENSG00000115946 | PNO1 |
| ENSG00000188647 | PTAR1 |
| ENSG00000146007 | ZMAT2 |
| ENSG00000241837 | ATP5O |
| ENSG00000113643 | RARS |
| ENSG00000162521 | RBBP4 |
| ENSG00000116830 | TTF2 |
| ENSG00000187555 | USP7 |
| ENSG00000137216 | TMEM63B |
| ENSG00000161904 | LEMD2 |
| ENSG00000241945 | PWP2 |
| ENSG00000134982 | APC |
| ENSG00000156983 | BRPF1 |
| ENSG00000164346 | NSA2 |
| ENSG00000223496 | EXOSC6 |
| ENSG00000113569 | NUP155 |
| ENSG00000080986 | NDC80 |
| ENSG00000143374 | TARS2 |
| ENSG00000104835 | SARS2 |
| ENSG00000152253 | SPC25 |
| ENSG00000088356 | PDRG1 |
| ENSG00000044574 | HSPA5 |
| ENSG00000116874 | WARS2 |
| ENSG00000204531 | POU5F1 |
| ENSG00000004779 | NDUFAB1 |
| ENSG00000161981 | SNRNP25 |
| ENSG00000126457 | PRMT1 |
| ENSG00000142507 | PSMB6 |
| ENSG00000164808 | SPIDR |
| ENSG00000234972 | TBC1D3C |
| ENSG00000144554 | FANCD2 |
| ENSG00000147383 | NSDHL |
| ENSG00000165732 | DDX21 |
| ENSG00000155975 | VPS37A |
| ENSG00000002822 | MAD1L1 |
| ENSG00000179271 | GADD45GIP1 |
| ENSG00000101452 | DHX35 |
| ENSG00000074071 | MRPS34 |
| ENSG00000169045 | HNRNPH1 |
| ENSG00000087510 | TFAP2C |
| ENSG00000105819 | PMPCB |
| ENSG00000204351 | SKIV2L |
| ENSG00000160783 | PMF1 |
| ENSG00000152234 | ATP5A1 |
| ENSG00000127463 | EMC1 |
| ENSG00000124228 | DDX27 |
| ENSG00000100319 | ZMAT5 |
| ENSG00000065183 | WDR3 |
| ENSG00000058272 | PPP1R12A |
| ENSG00000136628 | EPRS |
| ENSG00000163017 | ACTG2 |
| ENSG00000104884 | ERCC2 |
| ENSG00000166483 | WEE1 |
| ENSG00000135837 | CEP350 |
| ENSG00000104897 | SF3A2 |
| ENSG00000140598 | EFTUD1 |
| ENSG00000143774 | GUK1 |
| ENSG00000085721 | RRN3 |
| ENSG00000172053 | QARS |
| ENSG00000165934 | CPSF2 |
| ENSG00000052802 | MSMO1 |
| ENSG00000135476 | ESPL1 |
| ENSG00000174177 | CTU2 |
| ENSG00000120438 | TCP1 |
| ENSG00000170892 | TSEN34 |
| ENSG00000204574 | ABCF1 |
| ENSG00000175376 | EIF1AD |
| ENSG00000146263 | MMS22L |
| ENSG00000121022 | COPS5 |
| ENSG00000168090 | COPS6 |
| ENSG00000167491 | GATAD2A |
| ENSG00000084072 | PPIE |
| ENSG00000115268 | RPS15 |
| ENSG00000163938 | GNL3 |
| ENSG00000151665 | PIGF |
| ENSG00000148843 | PDCD11 |
| ENSG00000141736 | ERBB2 |
| ENSG00000103168 | TAF1C |
| ENSG00000105401 | CDC37 |
| ENSG00000163933 | RFT1 |
| ENSG00000122085 | MTERFD2 |
| ENSG00000164032 | H2AFZ |
| ENSG00000140943 | MBTPS1 |
| ENSG00000198952 | SMG5 |
| ENSG00000169021 | UQCRFS1 |
| ENSG00000013810 | TACC3 |
| ENSG00000105258 | POLR2I |
| ENSG00000167978 | SRRM2 |
| ENSG00000095564 | BTAF1 |
| ENSG00000138095 | LRPPRC |
| ENSG00000063978 | RNF4 |
| ENSG00000162368 | CMPK1 |
| ENSG00000140829 | DHX38 |
| ENSG00000158169 | FANCC |
| ENSG00000161960 | EIF4A1 |
| ENSG00000181222 | POLR2A |
| ENSG00000165916 | PSMC3 |
| ENSG00000198060 | MARCH5 |
| ENSG00000149923 | PPP4C |
| ENSG00000111667 | USP5 |
| ENSG00000198755 | RPL10A |
| ENSG00000141499 | WRAP53 |
| ENSG00000093009 | CDC45 |
| ENSG00000105732 | ZNF574 |
| ENSG00000104064 | GABPB1 |
| ENSG00000108294 | PSMB3 |
| ENSG00000130856 | ZNF236 |
| ENSG00000133980 | VRTN |
| ENSG00000149308 | NPAT |
| ENSG00000120071 | KANSL1 |
| ENSG00000129084 | PSMA1 |
| ENSG00000117877 | CD3EAP |
| ENSG00000127616 | SMARCA4 |
| ENSG00000163882 | POLR2H |
| ENSG00000183718 | TRIM52 |
| ENSG00000106803 | SEC61B |
| ENSG00000114942 | EEF1B2 |
| ENSG00000067704 | IARS2 |
| ENSG00000114686 | MRPL3 |
| ENSG00000172315 | TP53RK |
| ENSG00000173120 | KDM2A |
| ENSG00000138442 | WDR12 |
| ENSG00000145982 | FARS2 |
| ENSG00000117481 | NSUN4 |
| ENSG00000142676 | RPL11 |
| ENSG00000164615 | CAMLG |
| ENSG00000138073 | PREB |
| ENSG00000136888 | ATP6V1G1 |
| ENSG00000221829 | FANCG |
| ENSG00000198887 | SMC5 |
| ENSG00000102900 | NUP93 |
| ENSG00000108344 | PSMD3 |
| ENSG00000023191 | RNH1 |
| ENSG00000143621 | ILF2 |
| ENSG00000112855 | HARS2 |
| ENSG00000110536 | PTPMT1 |
| ENSG00000165629 | ATP5C1 |
| ENSG00000166847 | DCTN5 |
| ENSG00000104852 | SNRNP70 |
| ENSG00000203814 | HIST2H2BF |
| ENSG00000009413 | REV3L |
| ENSG00000130772 | MED18 |
| ENSG00000079313 | REXO1 |
| ENSG00000012061 | ERCC1 |
| ENSG00000111642 | CHD4 |
| ENSG00000100462 | PRMT5 |
| ENSG00000174100 | MRPL45 |
| ENSG00000101421 | CHMP4B |
| ENSG00000144028 | SNRNP200 |
| ENSG00000108592 | FTSJ3 |
| ENSG00000110048 | OSBP |
| ENSG00000147403 | RPL10 |
| ENSG00000198783 | ZNF830 |
| ENSG00000179409 | GEMIN4 |
| ENSG00000147604 | RPL7 |
| ENSG00000136824 | SMC2 |
| ENSG00000104889 | RNACEH2A |
| ENSG00000146282 | RARS2 |
| ENSG00000068784 | SRBD1 |
| ENSG00000137822 | TUBGCP4 |
| ENSG00000059691 | PET112 |
| ENSG00000066827 | ZFAT |
| ENSG00000148308 | GTF3C5 |
| ENSG00000170185 | USP38 |
| ENSG00000160201 | U2AF1 |
| ENSG00000141258 | SGSM2 |
| ENSG00000172660 | TAF15 |
| ENSG00000145833 | DDX46 |
| ENSG00000104980 | TIMM44 |
| ENSG00000097046 | CDC7 |
| ENSG00000131368 | MRPS25 |
| ENSG00000204209 | DAXX |
| ENSG00000129696 | TTI2 |
| ENSG00000108848 | LUC7L3 |
| ENSG00000013573 | DDX11 |
| ENSG00000105248 | CCDC94 |
| ENSG00000183598 | HIST2H3D |
| ENSG00000224226 | TBC1D3B |
| ENSG00000090470 | PDCD7 |
| ENSG00000031698 | SARS |
| ENSG00000108270 | AATF |
| ENSG00000159111 | MRPL10 |
| ENSG00000149806 | FAU |
| ENSG00000188739 | RBM34 |
| ENSG00000152684 | PELO |
| ENSG00000174374 | WBSCR16 |
| ENSG00000107036 | KIAA1432 |
| ENSG00000204619 | PPP1R11 |
| ENSG00000091651 | ORC6 |
| ENSG00000134480 | CCNH |
| ENSG00000164151 | KIAA0947 |
| ENSG00000164611 | PTTG1 |
| ENSG00000111445 | RFC5 |
| ENSG00000127481 | UBR4 |
| ENSG00000159352 | PSMD4 |
| ENSG00000137814 | HAUS2 |
| ENSG00000105220 | GPI |
| ENSG00000140521 | POLG |
| ENSG00000075856 | START3 |
| ENSG00000143742 | SRP9 |
| ENSG00000163029 | SMC6 |
| ENSG00000162227 | TAF6L |
| ENSG00000100129 | EIF3L |
| ENSG00000170348 | TMED10 |
| ENSG00000182214 | HIST2H4B |
| ENSG00000183941 | HIST2H4A |
| ENSG00000116221 | MRPL37 |
| ENSG00000196235 | SUPT5H |
| ENSG00000161920 | MED11 |
| ENSG00000134690 | CDCA8 |
| ENSG00000131153 | GINS2 |
| ENSG00000138018 | EPT1 |
| ENSG00000173141 | MRP63 |
| ENSG00000154727 | GABPA |
| ENSG00000120800 | UTP20 |
| ENSG00000114767 | RRP9 |
| ENSG00000174231 | PRPF8 |
| ENSG00000137547 | MRPL15 |
| ENSG00000146576 | C7orf26 |
| ENSG00000062568 | WDR18 |
| ENSG00000147162 | OGT |
| ENSG00000198917 | C9orf114 |
| ENSG00000180822 | PSMG4 |
| ENSG00000125977 | EIF2S2 |
| ENSG00000173418 | NAA20 |
| ENSG00000155561 | NUP205 |
| ENSG00000173545 | ZNF622 |
| ENSG00000127993 | RBM48 |
| ENSG00000197102 | DYNC1H1 |
| ENSG00000119392 | GLE1 |
| ENSG00000174444 | RPL4 |
| ENSG00000149716 | ORAOV1 |
| ENSG00000266876 | RRAGA |
| ENSG00000198841 | KTI12 |
| ENSG00000056097 | ZFR |
| ENSG00000227057 | WDR46 |
| ENSG00000167670 | CHAF1A |
| ENSG00000127191 | TRAF2 |
| ENSG00000072506 | HSD17B10 |
| ENSG00000215021 | PHB2 |
| ENSG00000175467 | SART1 |
| ENSG00000121073 | SLC35B1 |
| ENSG00000079459 | FDFT1 |
| ENSG00000143493 | INTS7 |
| ENSG0000014153 | EIF4A3 |
| ENSG00000174197 | MGA |
| ENSG00000131269 | ABCB7 |
| ENSG00000089009 | RPL6 |
| ENSG00000197780 | TAF13 |
| ENSG00000036549 | ZZZ3 |
| ENSG00000066135 | KDM4A |
| ENSG00000176473 | WDR25 |
| ENSG00000124614 | RPS10 |
| ENSG00000107581 | EIF3A |
| ENSG00000084463 | WBP11 |
| ENSG00000137656 | BUD13 |
| ENSG00000183751 | TBL3 |
| ENSG00000119537 | KDSR |
| ENSG00000204220 | PFDN6 |
| ENSG00000170291 | ELP5 |
| ENSG00000198563 | DDX39B |
| ENSG00000077549 | CAPZB |
| ENSG00000255529 | POLR2M |
| ENSG00000100034 | PPM1F |
| ENSG00000196367 | TRRAP |
| ENSG00000167258 | CDK12 |
| ENSG00000039123 | SKIV2L2 |
| ENSG00000076043 | REXO2 |
| ENSG00000213676 | ATF6B |
| ENSG00000058453 | CROCC |
| ENSG00000153575 | TUBGCP5 |
| ENSG00000110700 | RPS13 |
| ENSG00000101181 | MTG2 |
| ENSG00000071539 | TRIP13 |
| ENSG00000075702 | WDR62 |
| ENSG00000171453 | POLR1C |
| ENSG00000090989 | EXOC1 |
| ENSG00000037897 | METTL1 |
| ENSG00000095139 | ARCN1 |
| ENSG00000078142 | PIK3C3 |
| ENSG00000141030 | COPS3 |
| ENSG00000126249 | PDCD2L |
| ENSG00000117408 | IPO13 |
| ENSG00000130725 | UBE2M |
| ENSG00000175054 | ATR |
| ENSG00000149016 | TUT1 |
| ENSG00000165060 | FXN |
| ENSG00000117597 | DIEXF |
| ENSG00000185085 | INTS5 |
| ENSG00000113595 | TRIM23 |
| ENSG00000040633 | PHF23 |
| ENSG00000178952 | TUFM |
| ENSG00000120539 | MASTL |
| ENSG00000103549 | RNF40 |
| ENSG00000119723 | COQ6 |
| ENSG00000171311 | EXOSC1 |
| ENSG00000106245 | BUD31 |
| ENSG00000118046 | STK11 |
| ENSG00000125484 | GTF3C4 |
| ENSG00000089094 | KDM2B |
| ENSG00000121621 | KIF18A |
| ENSG00000129911 | KLF16 |
| ENSG00000102302 | FGD1 |
| ENSG00000135679 | MDM2 |
| ENSG00000185115 | NDNL2 |
| ENSG00000140553 | UNC45A |
| ENSG00000129562 | DAD1 |
| ENSG00000100138 | NHP2L1 |
| ENSG00000111641 | NOP2 |
| ENSG00000173660 | UQCRH |
| ENSG00000198677 | TTC37 |
| ENSG00000135503 | ACVR1B |
| ENSG00000180998 | GPR137C |
| ENSG00000153187 | HNRNPU |
| ENSG00000106459 | NRF1 |
| ENSG00000156261 | CCT8 |
| ENSG00000118363 | SPCS2 |
| ENSG00000164134 | NAA15 |
| ENSG00000060642 | PIGV |
| ENSG00000090889 | KIF4A |
| ENSG00000101361 | NOP56 |
| ENSG00000167792 | NDUFV1 |
| ENSG00000184162 | NR2C2AP |
| ENSG00000128524 | ATP6V1F |
| ENSG00000100387 | RBX1 |
| ENSG00000110906 | KCTD10 |
| ENSG00000147457 | CHMP7 |
| ENSG00000124570 | SERPINB6 |
| ENSG00000186468 | RPS23 |
| ENSG00000136122 | BORA |
| ENSG00000047249 | ATP6V1H |
| ENSG00000127804 | METTL16 |
| ENSG00000104412 | EMC2 |
| ENSG00000173726 | TOMM20 |
| ENSG00000138777 | PPA2 |
| ENSG00000170043 | TRAPPC1 |
| ENSG00000124486 | USP9X |
| ENSG00000105705 | SUGP1 |
| ENSG00000223501 | VPS52 |
| ENSG00000107815 | C10orf2 |
| ENSG00000100109 | TFIP11 |
| ENSG00000136271 | DDX56 |
| ENSG00000146830 | GIGYF1 |
| ENSG00000198382 | UVRAG |
| ENSG00000160285 | LSS |
| ENSG00000137770 | CTDSPL2 |
| ENSG00000116670 | MAD2L2 |
| ENSG00000165280 | VCP |
| ENSG00000183963 | SMTN |
| ENSG00000164961 | KIAA0196 |
| ENSG00000157216 | SSBP3 |
| ENSG00000129932 | DOHH |
| ENSG00000167721 | TSR1 |
| ENSG00000188352 | FOCAD |
| ENSG00000104853 | CLPTM1 |
| ENSG00000185883 | ATP6V0C |
| ENSG00000100519 | PSMC6 |
| ENSG00000110107 | PRPF19 |
| ENSG00000184203 | PPP1R2 |
| ENSG00000148824 | MTG1 |
| ENSG00000113810 | SMC4 |
| ENSG00000121152 | NCAPH |
| ENSG00000241127 | YAE1D1 |
| ENSG00000139197 | PEX5 |
| ENSG00000101464 | PIGU |
| ENSG00000132676 | DAP3 |
| ENSG00000135972 | MRPS9 |
| ENSG00000089157 | RPLP0 |
| ENSG00000138035 | PNPT1 |
| ENSG00000171824 | EXOSC10 |
| ENSG00000153179 | RASSF3 |
| ENSG00000110713 | NUP98 |
| ENSG00000100865 | CINP |
| ENSG00000136045 | PWP1 |
| ENSG00000167526 | RPL13 |
| ENSG00000088766 | CRLS1 |
| ENSG00000103510 | KAT8 |
| ENSG00000143368 | SF3B4 |
| ENSG00000156697 | UTP14A |
| ENSG00000176248 | ANAPC2 |
| ENSG00000188786 | MTF1 |
| ENSG00000175756 | AURKAIP1 |
| ENSG00000140395 | WDR61 |
| ENSG00000113368 | LMNB1 |
| ENSG00000060339 | CCAR1 |
| ENSG00000162385 | MAGOH |
| ENSG00000105372 | RPS19 |
| ENSG00000083312 | TNPO1 |
| ENSG00000100142 | POLR2F |
| ENSG00000204560 | DHX16 |
| ENSG00000197771 | MCMBP |
| ENSG00000099817 | POLR2E |
| ENSG00000161980 | POLR3K |
| ENSG00000117133 | RPF1 |
| ENSG00000125901 | MRPS26 |
| ENSG00000168827 | GFM1 |
| ENSG00000161513 | FDXR |
| ENSG00000137818 | RPLP1 |
| ENSG00000150990 | DHX37 |
| ENSG00000061794 | MRPS35 |
| ENSG00000143155 | TIPRL |
| ENSG00000253626 | EIF5AL1 |
| ENSG00000231500 | RPS18 |
| ENSG00000188076 | SCGB1C1 |
| ENSG00000174442 | ZWILCH |
| ENSG00000242028 | HYPK |
| ENSG00000124217 | MOCS3 |
| ENSG00000134186 | PRPF38B |
| ENSG00000105849 | TWISTNB |
| ENSG00000137337 | MDC1 |
| ENSG00000132207 | SLX1A |
| ENSG00000181625 | SLX1B |
| ENSG00000110717 | NDUFS8 |
| ENSG00000132341 | RAN |
| ENSG00000014123 | UFL1 |
| ENSG00000101191 | DIDO1 |
| ENSG00000125952 | MAX |
| ENSG00000163714 | U2SURP |
| ENSG00000253710 | ALG11 |
| ENSG00000104356 | POP1 |
| ENSG00000130826 | DKC1 |
| ENSG00000198780 | FAM169A |
| ENSG00000116688 | MFN2 |
| ENSG00000166166 | TRMT61A |
| ENSG00000214517 | PPME1 |
| ENSG00000077253 | GTF3C1 |
| ENSG00000152240 | HAUS1 |
| ENSG00000063177 | RPL18 |
| ENSG00000087157 | PGS1 |
| ENSG00000100567 | PSMA3 |
| ENSG00000169371 | SNUPN |
| ENSG00000197651 | CCER1 |
| ENSG00000198900 | TOP1 |
| ENSG00000213551 | DNAJC9 |
| ENSG00000152464 | RPP38 |
| ENSG00000131467 | PSME3 |
| ENSG00000223510 | CDRT15 |
| ENSG00000115053 | NCL |
| ENSG00000163041 | H3F3A |
| ENSG00000154813 | DPH3 |
| ENSG00000181873 | IBA57 |
| ENSG00000185591 | SP1 |
| ENSG00000115355 | CCDC88A |
| ENSG00000139350 | NEDD1 |
| ENSG00000108518 | PFN1 |
| ENSG00000108264 | TADA2A |
| ENSG00000134809 | TIMM10 |
| ENSG00000124383 | MPHOSPH10 |
| ENSG00000126067 | PSMB2 |
| ENSG00000060688 | SNRNP40 |
| ENSG00000042429 | MED17 |
| ENSG00000196655 | TRAPPC4 |
| ENSG00000107185 | RGP1 |
| ENSG00000124608 | AARS2 |
| ENSG00000092098 | RNF31 |
| ENSG00000143569 | UBAP2L |
| ENSG00000233822 | HIST1H2BN |
| ENSG00000171848 | RRM2 |
| ENSG00000183161 | FANCF |
| ENSG00000166197 | NOLC1 |
| ENSG00000064703 | DDX20 |
| ENSG00000176102 | CSTF3 |
| ENSG00000106028 | SSBP1 |
| ENSG00000143315 | PIGM |
| ENSG00000136152 | COG3 |
| ENSG00000134697 | GNL2 |
| ENSG00000159217 | IGF2BP1 |
| ENSG00000080608 | KIAA0020 |
| ENSG00000267368 | UPK3BL |
| ENSG00000130119 | GNL3L |
| ENSG00000178950 | GAK |
| ENSG00000205659 | LIN52 |
| ENSG00000123297 | TSFM |
| ENSG00000241370 | RPP21 |
| ENSG00000129351 | ILF3 |
| ENSG00000174446 | SNAPC5 |
| ENSG00000132382 | MYBBP1A |
| ENSG00000100664 | EIF5 |
| ENSG00000131469 | RPL27 |
| ENSG00000185128 | TBC1D3F |
| ENSG00000111231 | GPN3 |
| ENSG00000182774 | RPS17L |
| ENSG00000184779 | RPS17 |
| ENSG00000186871 | ERCC6L |
| ENSG00000204568 | MRPS18B |
| ENSG00000108312 | UBTF |
| ENSG00000167965 | MLST8 |
| ENSG00000115241 | PPM1G |
| ENSG00000171103 | TRMT61B |
| ENSG00000116586 | LAMTOR2 |
| ENSG00000105793 | GTPBP10 |
| ENSG00000100348 | TXN2 |
| ENSG00000172757 | CFL1 |
| ENSG00000163634 | THOC7 |
| ENSG00000008324 | SS18L2 |
| ENSG00000152404 | CWF19L2 |
| ENSG00000020129 | NCDN |
| ENSG00000181449 | SOX2 |
| ENSG00000136997 | MYC |
| ENSG00000175166 | PSMD2 |
| ENSG00000070614 | NDST1 |
| ENSG00000115484 | CCT4 |
| ENSG00000100890 | KIAA0391 |
| ENSG00000149474 | CSRP2BP |
| ENSG00000102738 | MRPS31 |
| ENSG00000136104 | RNASEH2B |
| ENSG00000106246 | PTCD1 |
| ENSG00000248919 | ATP5J2-PTCD1 |
| ENSG00000138663 | COPS4 |
| ENSG00000115368 | WDR75 |
| ENSG00000128564 | VGF |
| ENSG00000128191 | DGCR8 |
| ENSG00000008294 | SPAG9 |
| ENSG00000131475 | VPS25 |
| ENSG00000105523 | FAM83E |
| ENSG00000172269 | DPAGT1 |
| ENSG00000170312 | CDK1 |
| ENSG00000104131 | EIF3J |
| ENSG00000150753 | CCT5 |
| ENSG00000140443 | IGF1R |
| ENSG00000010292 | NCAPD2 |
| ENSG00000171763 | SPATA5L1 |
| ENSG00000180098 | TRNAU1AP |
| ENSG00000168374 | ARF4 |
| ENSG00000173812 | EIF1 |
| ENSG00000100554 | ATP6V1D |
| ENSG00000072756 | TRNT1 |
| ENSG00000135372 | NAT10 |
| ENSG00000178394 | HTR1A |
| ENSG00000128272 | ATF4 |
| ENSG00000204070 | SYS1 |
| ENSG00000137815 | RTF1 |
| ENSG00000198026 | ZNF335 |
| ENSG00000117410 | ATP6V0B |
| ENSG00000112739 | PRPF4B |
| ENSG00000129347 | KRI1 |
| ENSG00000221818 | EBF2 |
| ENSG00000198431 | TXNRD1 |
| ENSG00000104979 | C19orf53 |
| ENSG00000136709 | WDR33 |
| ENSG00000149100 | EIF3M |
| ENSG00000125835 | SNRPB |
| ENSG00000116698 | SMG7 |
| ENSG00000087586 | AURKA |
| ENSG00000169230 | PRELID1 |
| ENSG00000143799 | PARP1 |
| ENSG00000146731 | CCT6A |
| ENSG00000163877 | SNIP1 |
| ENSG00000215421 | ZNF407 |
| ENSG00000197724 | PHF2 |
| ENSG00000172590 | MRPL52 |
| ENSG00000175203 | DCTN2 |
| ENSG00000149273 | RPS3 |
| ENSG00000204822 | MRPL53 |
| ENSG00000109775 | UFSP2 |
| ENSG00000165733 | BMS1 |
| ENSG00000104671 | DCTN6 |
| ENSG00000175224 | ATG13 |
| ENSG00000142541 | RPL13A |
| ENSG00000173805 | HAP1 |
| ENSG00000115750 | TAF1B |
| ENSG00000165688 | PMPCA |
| ENSG00000159720 | ATP6V0D1 |
| ENSG00000074201 | CLNS1A |
| ENSG00000158417 | EIF5B |
| ENSG00000196588 | MKL1 |
| ENSG00000138614 | VWA9 |
| ENSG00000124571 | XPO5 |
| ENSG00000198000 | NOL8 |
| ENSG00000181991 | MRPS11 |
| ENSG00000149823 | VPS51 |
| ENSG00000151348 | EXT2 |
| ENSG00000162396 | PARS2 |
| ENSG00000204843 | DCTN1 |
| ENSG00000177302 | TOP3A |
| ENSG00000142684 | ZNF593 |
| ENSG00000074800 | ENO1 |
| ENSG00000167513 | CDT1 |
| ENSG00000141101 | NOB1 |
| ENSG00000047315 | POLR2B |
| ENSG00000131966 | ACTR10 |
| ENSG00000115875 | SRSF7 |
| ENSG00000186141 | POLR3C |
| ENSG00000108424 | KPNB1 |
| ENSG00000111845 | PAK1IP1 |
| ENSG00000148832 | PAOX |
| ENSG00000156017 | C9orf41 |
| ENSG00000198901 | PRC1 |
| ENSG00000134001 | EIF2S1 |
| ENSG00000146918 | NCAPG2 |
| ENSG00000144713 | RPL32 |
| ENSG00000185122 | HSF1 |
| ENSG00000167658 | EEF2 |
| ENSG00000164190 | NIPBL |
| ENSG00000163902 | RPN1 |
| ENSG00000244045 | TMEM199 |
| ENSG00000143476 | DTL |
| ENSG00000149503 | INCENP |
| ENSG00000071243 | ING3 |
| ENSG00000186073 | C15orf41 |
| ENSG00000088836 | SLC4A11 |
| ENSG00000136273 | HUS1 |
| ENSG00000005007 | UPF1 |
| ENSG00000070010 | UFD1L |
| ENSG00000106263 | EIF3B |
| ENSG00000213024 | NUP62 |
| ENSG00000067191 | CACNB1 |
| ENSG00000179091 | CYC1 |
| ENSG00000113312 | TTC1 |
| ENSG00000085831 | TTC39A |
| ENSG00000118197 | DDX59 |
| ENSG00000134871 | COL4A2 |
| ENSG00000088986 | DYNLL1 |
| ENSG00000138778 | CENPE |
| ENSG00000106244 | PDAP1 |
| ENSG00000177600 | RPLP2 |
| ENSG00000112081 | SRSF3 |
| ENSG00000100413 | POLR3H |
| ENSG00000172508 | CARNS1 |
| ENSG00000147123 | NDUFB11 |
| ENSG00000119953 | SMNDC1 |
| ENSG00000111640 | GAPDH |
| ENSG00000117899 | MESDC2 |
| ENSG00000075624 | ACTB |
| ENSG00000163166 | IWS1 |
| ENSG00000114503 | NCBP2 |
| ENSG00000198522 | GPN1 |
| ENSG00000099899 | TRMT2A |
| ENSG00000181544 | FANCB |
| ENSG00000136982 | DSCC1 |
| ENSG00000068366 | ACSL4 |
| ENSG00000062716 | VMP1 |
| ENSG00000111802 | TDP2 |
| ENSG00000185627 | PSMD13 |
| ENSG00000020426 | MNAT1 |
| ENSG00000113734 | BNIP1 |
| ENSG00000102241 | HTATSF1 |
| ENSG00000160789 | LMNA |
| ENSG00000062822 | POLD1 |
| ENSG00000168944 | CEP120 |
| ENSG00000139718 | SETD1B |
| ENSG00000132792 | CTNNBL1 |
| ENSG00000173540 | GMPPB |
| ENSG00000128789 | PSMG2 |
| ENSG00000196365 | LONP1 |
| ENSG00000160214 | RRP1 |
| ENSG00000179041 | RRS1 |
| ENSG00000143106 | PSMA5 |
| ENSG00000168411 | RFWD3 |
| ENSG00000073584 | SMARCE1 |
| ENSG00000175334 | BANF1 |
| ENSG00000077152 | UBE2T |
| ENSG00000173611 | SCAI |
| ENSG00000171720 | HDAC3 |
| ENSG00000182197 | EXT1 |
| ENSG00000114346 | ECT2 |
| ENSG00000124214 | STAU1 |
| ENSG00000126254 | RBM42 |
| ENSG00000127184 | COX7C |
| ENSG00000174276 | ZNHIT2 |
| ENSG00000177971 | IMP3 |
| ENSG00000104872 | PIH1D1 |
| ENSG00000132155 | RAF1 |
| ENSG00000163872 | YEATS2 |
| ENSG00000119906 | FAM178A |
| ENSG00000217930 | PAM16 |
| ENSG00000197498 | RPF2 |
| ENSG00000130348 | QRSL1 |
| ENSG00000147536 | GINS4 |
| ENSG00000174748 | RPL15 |
| ENSG00000159147 | DONSON |
| ENSG00000157593 | SLC35B2 |
| ENSG00000181938 | GINS3 |
| ENSG00000187446 | CHP1 |
| ENSG00000070371 | CLTCL1 |
| ENSG00000096063 | SRPK1 |
| ENSG00000141564 | RPTOR |
| ENSG00000108474 | PIGL |
| ENSG00000187741 | FANCA |
| ENSG00000213465 | ARL2 |
| ENSG00000117593 | DARS2 |
| ENSG00000171863 | RPS7 |
| ENSG00000117395 | EBNA1BP2 |
| ENSG00000111142 | METAP2 |
| ENSG00000113272 | THG1L |
| ENSG00000117360 | PRPF3 |
| ENSG00000221978 | CCNL2 |
| ENSG00000163832 | ELP6 |
| ENSG00000108852 | MPP2 |
| ENSG00000175832 | ETV4 |
| ENSG00000185359 | HGS |
| ENSG00000120705 | ETF1 |
| ENSG00000108384 | RAD51C |
| ENSG00000036257 | CUL3 |
| ENSG00000152382 | TADA1 |
| ENSG00000114742 | WDR48 |
| ENSG00000214026 | MRPL23 |
| ENSG00000105671 | DDX49 |
| ENSG00000104731 | KLHDC4 |
| ENSG00000010256 | UQCRC1 |
| ENSG00000154743 | TSEN2 |
| ENSG00000178896 | EXOSC4 |
| ENSG00000168393 | DTYMK |
| ENSG00000035928 | RFC1 |
| ENSG00000048707 | VPS13D |
| ENSG00000154832 | CXXC1 |
| ENSG00000130985 | UBA1 |
| ENSG00000065150 | IPO5 |
| ENSG00000161800 | RACGAP1 |
| ENSG00000142534 | RPS11 |
| ENSG00000136003 | ISCU |
| ENSG00000065000 | AP3D1 |
| ENSG00000100401 | RANGAP1 |
| ENSG00000196230 | TUBB |
| ENSG00000181555 | SETD2 |
| ENSG00000055950 | MRPL43 |
| ENSG00000188389 | PDCD1 |
| ENSG00000165684 | SNAPC4 |
| ENSG00000147533 | GOLGA7 |
| ENSG00000064313 | TAF2 |
| ENSG00000137154 | RPS6 |
| ENSG00000104886 | PLEKHJ1 |
| ENSG00000122882 | ECD |
| ENSG00000184967 | NOC4L |
| ENSG00000088325 | TPX2 |
| ENSG00000183520 | UTP11L |
| ENSG00000179051 | RCC2 |
| ENSG00000157510 | AFAP1L1 |
| ENSG00000066379 | ZNRD1 |
| ENSG00000172115 | CYCS |
| ENSG00000086827 | ZW10 |
| ENSG00000109534 | GAR1 |
| ENSG00000175387 | SMAD2 |
| ENSG00000115947 | ORC4 |
| ENSG00000010072 | SPRTN |
| ENSG00000185163 | DDX51 |
| ENSG00000177370 | TIMM22 |
| ENSG00000076924 | XAB2 |
| ENSG00000124562 | SNRPC |
| ENSG00000127586 | CHTF18 |
| ENSG00000066117 | SMARCD1 |
| ENSG00000177494 | ZBED2 |
| ENSG00000133401 | PDZD2 |
| ENSG00000127554 | GFER |
| ENSG00000117697 | NSL1 |
| ENSG00000184659 | FOXD4L4 |
| ENSG00000204828 | FOXD4L2 |
| ENSG00000110200 | ANAPC15 |
| ENSG00000169291 | SHE |
| ENSG00000132313 | MRPL35 |
| ENSG00000115816 | CEBPZ |
| ENSG00000243667 | WDR92 |
| ENSG00000107959 | PITRM1 |
| ENSG00000103035 | PSMD7 |
| ENSG00000163946 | FAM208A |
| ENSG00000178057 | NDUFAF3 |
| ENSG00000170540 | ARL6IP1 |
| ENSG00000091009 | RBM27 |
| ENSG00000205609 | EIF3CL |
| ENSG00000165526 | RPUSD4 |
| ENSG00000120314 | WDR55 |
| ENSG00000013275 | PSMC4 |
| ENSG00000131931 | THAP1 |
| ENSG00000155660 | PDIA4 |
| ENSG00000162607 | USP1 |
| ENSG00000109606 | DHX15 |
| ENSG00000261949 | LOC100507003 |
| ENSG00000130589 | HELZ2 |
| ENSG00000145734 | BDP1 |
| ENSG00000103194 | USP10 |
| ENSG00000076201 | PTPN23 |
| ENSG00000140854 | KATNB1 |
| ENSG00000164053 | ATRIP |
| ENSG00000167088 | SNRPD1 |
| ENSG00000154781 | CCDC174 |
| ENSG00000115446 | UNC50 |
| ENSG00000177700 | POLR2L |
| ENSG00000162063 | CCNF |
| ENSG00000152904 | GGPS1 |
| ENSG00000151657 | KIN |
| ENSG00000182810 | DDX28 |
| ENSG00000006744 | ELAC2 |
| ENSG00000116898 | MRPS15 |
| ENSG00000255072 | PIGY |
| ENSG00000130332 | LSM7 |
| ENSG00000051180 | RAD51 |
| ENSG00000178171 | AMER3 |
| ENSG00000254901 | MEF2BNB |
| ENSG00000149925 | ALDOA |
| ENSG00000100604 | CHGA |
| ENSG00000172602 | RND1 |
| ENSG00000138592 | USP8 |
| ENSG00000172613 | RAD9A |
| ENSG00000132196 | HSD17B7 |
| ENSG00000151849 | CENPJ |
| ENSG00000105221 | AKT2 |
| ENSG00000185504 | C17orf70 |
| ENSG00000025796 | SEC63 |
| ENSG00000168438 | CDC40 |
| ENSG00000163918 | RFC4 |
| ENSG00000152147 | GEMIN6 |
| ENSG00000166887 | VPS39 |
| ENSG00000018625 | ATP1A2 |
| ENSG00000163346 | PBXIP1 |
| ENSG00000135966 | TGFBRAP1 |
| ENSG00000099901 | RANBP1 |
| ENSG00000010327 | STAB1 |
| ENSG00000163344 | PMVK |
| ENSG00000102921 | N4BP1 |
| ENSG00000177150 | FAM210A |
| ENSG00000158042 | MRPL17 |
| ENSG00000124659 | TBCC |
| ENSG00000113593 | PPWD1 |
| ENSG00000188306 | LRRIQ4 |
| ENSG00000074966 | TXK |
| ENSG00000228049 | POLR2J2 |
| ENSG00000133226 | SRRM1 |
| ENSG00000121577 | POPDC2 |
| ENSG00000130876 | SLC7A10 |
| ENSG00000130810 | PPAN |
| ENSG00000243207 | PPAN-P2RY11 |
| ENSG00000081248 | CACNA1S |
| ENSG00000153201 | RANBP2 |
| ENSG00000126698 | DNAJC8 |
| ENSG00000103018 | CYB5B |
| ENSG00000130816 | DNMT1 |
| ENSG00000102103 | PQBP1 |
| ENSG00000120253 | NUP43 |
| ENSG00000164327 | RICTOR |
| ENSG00000139719 | VPS33A |
| ENSG00000168566 | SNRNP48 |
| ENSG00000063244 | U2AF2 |
| ENSG00000108423 | TUBD1 |
| ENSG00000164880 | INTS1 |
| ENSG00000148297 | MED22 |
| ENSG00000185825 | BCAP31 |
| ENSG00000084623 | EIF3I |
| ENSG00000066422 | ZBTB11 |
| ENSG00000119041 | GTF3C3 |
| ENSG00000083093 | PALB2 |
| ENSG00000120699 | EXOSC8 |
| ENSG00000166135 | HIF1AN |
| ENSG00000188976 | NOC2L |
| ENSG00000102974 | CTCF |
| ENSG00000148229 | POLE3 |
| ENSG00000167118 | URM1 |
| ENSG00000176386 | CDC26 |
| ENSG00000110063 | DCPS |
| ENSG00000089737 | DDX24 |
| ENSG00000119383 | PPP2R4 |
| ENSG00000143319 | ISG20L2 |
| ENSG00000141552 | ANAPC11 |
| ENSG00000155506 | LARP1 |
| ENSG00000144867 | SRPRB |
| ENSG00000093000 | NUP50 |
| ENSG00000107937 | GTPBP4 |
| ENSG00000083635 | NUFIP1 |
| ENSG00000174527 | MYO1H |
| ENSG00000124641 | MED20 |
| ENSG00000240694 | PNMA2 |
| ENSG00000122012 | SV2C |
| ENSG00000017260 | ATP2C1 |
| ENSG00000179965 | ZNF771 |
| ENSG00000126216 | TUBGCP3 |
| ENSG00000126814 | TRMT5 |
| ENSG00000101945 | SUB39H1 |
| ENSG00000182185 | RAD51B |
| ENSG00000163681 | SLMAP |
| ENSG00000179295 | PTPN11 |
| ENSG00000004487 | KDM1A |
| ENSG00000136100 | VPS36 |
| ENSG00000168066 | SF1 |
| ENSG00000197181 | PIWIL2 |
| ENSG00000128908 | INO80 |
| ENSG00000102144 | PGK1 |
| ENSG00000007923 | DNAJC11 |
| ENSG00000143514 | TP53BP2 |
| ENSG00000076650 | GPATCH1 |
| ENSG00000130749 | ZC3H4 |
| ENSG00000062582 | MRPS24 |
| ENSG00000087085 | ACHE |
| ENSG00000197976 | AKAP17A |
| ENSG00000100028 | SNRPD3 |
| ENSG00000128731 | HERC2 |
| ENSG00000134014 | ELP3 |
| ENSG00000181163 | NPM1 |
| ENSG00000148444 | COMMD3 |
| ENSG00000095319 | NUP188 |
| ENSG00000169564 | PCBP1 |
| ENSG00000182208 | MOB2 |
| ENSG00000055070 | SZRD1 |
| ENSG00000182473 | EXOC7 |
| ENSG00000136930 | PSMB7 |
| ENSG00000107863 | ARHGAP21 |
| ENSG00000197223 | C1D |
| ENSG00000184270 | HIST2H2AB |
| ENSG00000161036 | LRWD1 |
| ENSG00000144736 | SHQ1 |
| ENSG00000137100 | DCTN3 |
| ENSG00000131149 | GSE1 |
| ENSG00000214753 | HNRNPUL2 |
| ENSG00000111358 | GTF2H3 |
| ENSG00000147677 | EIF3H |
| ENSG00000125676 | THOC2 |
| ENSG00000149554 | CHEK1 |
| ENSG00000176476 | CCDC101 |
| ENSG00000147596 | PRDM14 |
| ENSG00000092094 | OSGEP |
| ENSG00000155393 | HEATR3 |
| ENSG00000083845 | RPS5 |
| ENSG00000148296 | SURF6 |
| ENSG00000162613 | FUBP1 |
| ENSG00000182220 | ATP6AP2 |
| ENSG00000115163 | CENPA |
| ENSG00000176225 | RTTN |
| ENSG00000176208 | ATAD5 |
| ENSG00000254827 | SLC22A18AS |
| ENSG00000128708 | HAT1 |
| ENSG00000106400 | ZNHIT1 |
| ENSG00000123219 | CENPK |
| ENSG00000264424 | MYH4 |
| ENSG00000066468 | FGFR2 |
| ENSG00000095059 | DHPS |
| ENSG00000110921 | MVK |
| ENSG00000141556 | TBCD |
| ENSG00000196305 | IARS |
| ENSG00000131055 | COX4I2 |
| ENSG00000153789 | FAM92B |
| ENSG00000088930 | XRN2 |
| ENSG00000145220 | LYAR |
| ENSG00000172809 | RPL38 |
| ENSG00000108788 | MLX |
| ENSG00000197170 | PSMD12 |
| ENSG00000225899 | FRG2B |
| ENSG00000174886 | NDUFA11 |
| ENSG00000172058 | SERF1A |
| ENSG00000205572 | SERF1B |
| ENSG00000242485 | MRPL20 |
| ENSG00000089225 | TBX5 |
| ENSG00000149428 | HYOU1 |
| ENSG00000166595 | FAM96B |
| ENSG00000131462 | TUBG1 |
| ENSG00000185990 | F8A3 |
| ENSG00000197932 | F8A1 |
| ENSG00000198444 | F8A2 |
| ENSG00000031823 | RANBP3 |
| ENSG00000100353 | EIF3D |
| ENSG00000163605 | PPP4R2 |
| ENSG00000164162 | ANAPC10 |
| ENSG00000132153 | DHX30 |
| ENSG00000154723 | ATP5J |
| ENSG00000182256 | GABRG3 |
| ENSG00000119487 | MAPKAP1 |
| ENSG00000132394 | EEFSEC |
| ENSG00000122952 | ZWINT |
| ENSG00000131042 | LILRB2 |
| ENSG00000222004 | C7orf71 |
| ENSG00000168802 | CHTF8 |
| ENSG00000069849 | ATP1B3 |
| ENSG00000074582 | BCS1L |
| ENSG00000103126 | AXIN1 |
| ENSG00000187144 | SPATA21 |
| ENSG00000221914 | PPP2R2A |
| ENSG00000163386 | NBPF10 |
| ENSG00000134987 | WDR36 |
| ENSG00000132300 | PTCD3 |
| ENSG00000156931 | VPS8 |
| ENSG00000165632 | TAF3 |
| ENSG00000044115 | CTNNA1 |
| ENSG00000035403 | VCL |
| ENSG00000088256 | GNA11 |
| ENSG00000164334 | FAM170A |
| ENSG00000166225 | FRS2 |
| ENSG00000241186 | TDGF1 |
| ENSG00000196374 | HIST1H2BM |
| ENSG00000117614 | SYF2 |
| ENSG00000154222 | CC2D1B |
| ENSG00000101367 | MAPRE1 |
| ENSG00000188186 | LAMTOR4 |
| ENSG00000166924 | NYAP1 |
| ENSG00000079805 | DNM2 |
| ENSG00000011260 | UTP18 |
| ENSG00000089685 | BIRC5 |
| ENSG00000123908 | AGO2 |
| ENSG00000057935 | MTA3 |
| ENSG00000100811 | YY1 |
| ENSG00000064102 | ASUN |
| ENSG00000006025 | OSBPL7 |
| ENSG00000107372 | ZFAND5 |
| ENSG00000172922 | RNASEH2C |
| ENSG00000075089 | ACTR6 |
| ENSG00000165119 | HNRNPK |
| ENSG00000182518 | FAM104B |
| ENSG00000041802 | LSG1 |
| ENSG00000206557 | TRIM71 |
| ENSG00000124140 | SLC12A5 |
| ENSG00000063046 | EIF4B |
| ENSG00000126581 | BECN1 |
| ENSG00000171530 | TBCA |
| ENSG00000206127 | GOLGA8O |
| ENSG00000167842 | MIS12 |
| ENSG00000033011 | ALG1 |
| ENSG00000146670 | CDCA5 |
| ENSG00000198856 | OSTC |
| ENSG00000111605 | CPSF6 |
| ENSG00000087365 | SF3B2 |
| ENSG00000135845 | PIGC |
| ENSG00000100220 | RTCB |
| ENSG00000131876 | SNRPA1 |
| ENSG00000115392 | FANCL |
| ENSG00000078618 | NRD1 |
| ENSG00000025770 | NCAPH2 |
| ENSG00000117682 | DHDDS |
| ENSG00000198844 | ARHGEF15 |
| ENSG00000132603 | NIP7 |
| ENSG00000162377 | SELRC1 |
| ENSG00000137411 | VARS2 |
| ENSG00000064886 | CHI3L2 |
| ENSG00000137806 | NDUFAF1 |
| ENSG00000133030 | MPRIP |
| ENSG00000136935 | GOLGA1 |
| ENSG00000243927 | MRPS6 |
| ENSG00000046647 | GEMIN8 |
| ENSG00000133124 | IRS4 |
| ENSG00000255346 | NOX5 |
| ENSG00000103275 | UBE2I |
| ENSG00000165502 | RPL36AL |
| ENSG00000100056 | DGCR14 |
| ENSG00000167972 | ABCA3 |
| ENSG00000053372 | MRTO4 |
| ENSG00000169813 | HNRNPF |
| ENSG00000198258 | UBL5 |
| ENSG00000103245 | NARFL |
| ENSG00000183513 | COA5 |
| ENSG00000174547 | MRPL11 |
| ENSG00000173457 | PPP1R14B |
| ENSG00000088038 | CNOT3 |
| ENSG00000115539 | PDCL3 |
| ENSG00000118181 | RPS25 |
| ENSG00000160075 | SSU72 |
| ENSG00000257949 | TEN1 |
| ENSG00000168028 | RPSA |
| ENSG00000213066 | FGFR1OP |
| ENSG00000143228 | NUF2 |
| ENSG00000137413 | TAF8 |
| ENSG00000124207 | CSE1L |
| ENSG00000080815 | PSEN1 |
| ENSG00000132773 | TOE1 |
| ENSG00000129460 | NGDN |
| ENSG00000188613 | NANOS1 |
| ENSG00000163636 | PSMD6 |
| ENSG00000146232 | NFKBIE |
| ENSG00000135902 | CHRND |
| ENSG00000143641 | GALNT2 |
| ENSG00000073969 | NSF |
| ENSG00000041982 | TNC |
| ENSG00000108256 | NUFIP2 |
| ENSG00000198911 | SREBF2 |
| ENSG00000141385 | AFG3L2 |
| ENSG00000176108 | CHMP6 |
| ENSG00000257365 | FNTB |
| ENSG00000186487 | MYT1L |
| ENSG00000127423 | AUNIP |
| ENSG00000112110 | MRPL18 |
| ENSG00000114650 | SCAP |
| ENSG00000178104 | PDE4DIP |
| ENSG00000105656 | ELL |
| ENSG00000186393 | KRT26 |
| ENSG00000124541 | RRP36 |
| ENSG00000182108 | DEXI |
| ENSG00000139133 | ALG10 |
| ENSG00000082068 | WDR70 |
| ENSG00000151388 | ADAMTS12 |
| ENSG00000172172 | MRPL13 |
| ENSG00000184979 | USP18 |
| ENSG00000239857 | GET4 |
| ENSG00000069345 | DNAJA2 |
| ENSG00000073050 | XRCC1 |
| ENSG00000070985 | TRPM5 |
| ENSG00000158715 | SLC45A3 |
| ENSG00000172062 | SMN1 |
| ENSG00000205571 | SMN2 |
| ENSG00000113141 | IK |
| ENSG00000186105 | LRRC70 |
| ENSG00000157895 | C12orf43 |
| ENSG00000166441 | RPL27A |
| ENSG00000106346 | USP42 |
| ENSG00000185379 | RAD51D |
| ENSG00000116667 | C1orf21 |
| ENSG00000176444 | CLK2 |
| ENSG00000105472 | CLEC11A |
| ENSG00000065613 | SLK |
| ENSG00000005156 | LIG3 |
| ENSG00000125459 | MSTO1 |
| ENSG00000139146 | FAM60A |
| ENSG00000060069 | CTDP1 |
| ENSG00000130935 | NOL11 |
| ENSG00000115677 | HDLBP |
| ENSG00000105254 | TBCB |
| ENSG00000075539 | FRYL |
| ENSG00000196747 | HIST1H2AI |
| ENSG00000181513 | ACBD4 |
| ENSG00000153107 | ANAPC1 |
| ENSG00000160211 | G6PD |
| ENSG00000111481 | COPZ1 |
| ENSG00000070761 | C16orf80 |
| ENSG00000168924 | LETM1 |
| ENSG00000105058 | FAM32A |
| ENSG00000204569 | PPP1R10 |
| ENSG00000153914 | SREK1 |
| ENSG00000161509 | GRIN2C |
| ENSG00000162702 | ZNF281 |
| ENSG00000007939 | SLC4A1 |
| ENSG00000139620 | KANSL2 |
| ENSG00000025293 | PHF20 |
| ENSG00000158545 | ZC3H18 |
| ENSG00000142546 | NOSIP |
| ENSG00000143398 | PIP5K1A |
| ENSG00000197958 | RPL12 |
| ENSG00000067225 | PKM |
| ENSG00000172534 | HCFC1 |
| ENSG00000155438 | MKI67IP |
| ENSG00000166582 | CENPV |
| ENSG00000145912 | NHP2 |
| ENSG00000180992 | MRPL14 |
| ENSG00000118705 | RPN2 |
| ENSG00000163161 | ERCC3 |
| ENSG00000136819 | C9orf78 |
| ENSG00000124787 | RPP40 |
| ENSG00000179104 | TMTC2 |
| ENSG00000140694 | PARN |
| ENSG00000143751 | SDE2 |
| ENSG00000136997 | MYC |
| ENSG00000147274 | RBMX |
| ENSG00000084693 | AGBL5 |
| ENSG00000165271 | NOL6 |
| ENSG00000221838 | AP4M1 |
| ENSG00000171444 | MCC |
| ENSG00000101882 | NKAP |
| ENSG00000186847 | KRT14 |
| ENSG00000014824 | SLC30A9 |
| ENSG00000166685 | COG1 |
| ENSG00000108349 | CASC3 |
| ENSC00000175216 | CKAP5 |
| ENSG00000259494 | MRPL46 |
| ENSG00000028310 | BRD9 |
| ENSG00000136450 | SRSF1 |
| ENSG00000204859 | ZBTB48 |
| ENSG00000165209 | STRBP |
| ENSG00000163466 | ARPC2 |
| ENSG00000125485 | DDX31 |
| ENSG00000070778 | PTPN21 |
| ENSG00000126001 | CEP250 |
| ENSG00000169249 | ZRSR2 |
| ENSG00000111011 | RSRC2 |
| ENSG00000139496 | NUPL1 |
| ENSG00000131746 | TNS4 |
| ENSG00000061936 | SFSWAP |
| ENSG00000196584 | XRCC2 |
| ENSG00000168286 | THAP11 |
| ENSG00000119787 | ATL2 |
| ENSG00000182446 | NPLOC4 |
| ENSG00000071462 | WBSCR22 |
| ENSG00000213397 | HAUS7 |
| ENSG00000178028 | DMAP1 |
| ENSG00000067596 | DHX8 |
| ENSG00000198015 | MRPL42 |
| ENSG00000133706 | LARS |
| ENSG00000149635 | OCSTAMP |
| ENSG00000117505 | DR1 |
| ENSG00000155868 | MED7 |
| ENSG00000129197 | RPAIN |
| ENSG00000065978 | YBX1 |
| ENSG00000260238 | PMF1-BGLAP |
| ENSG00000178988 | MRFAP1L1 |
| ENSG00000168005 | C11orf84 |
| ENSG00000162408 | NOL9 |
| ENSG00000140350 | ANP32A |
| ENSG00000261796 | ISY1-RAB43 |
| ENSG00000174405 | LIG4 |
| ENSG00000197414 | GOLGA6L1 |
| ENSG00000116062 | MSH6 |
| ENSG00000116906 | GNPAT |
| ENSG00000134597 | RBMX2 |
| ENSG00000071994 | PDCD2 |
| ENSG00000112742 | TTK |
| ENSG00000106636 | YKT6 |
| ENSG00000101773 | RBBP8 |
| ENSG00000103061 | SLC7A6OS |
| ENSG00000140259 | MFAP1 |
| ENSG00000197077 | KIAA1671 |
| ENSG00000204435 | CSNK2B |
| ENSG00000055130 | CUL1 |
| ENSG00000100209 | HSCB |
| ENSG00000113048 | MRPS27 |
| ENSG00000189403 | HMGB1 |
| ENSG00000173011 | TADA2B |
| ENSG00000169836 | TACR3 |
| ENSG00000133816 | MICAL2 |
| ENSG00000141452 | C18orf8 |
| ENSG00000006715 | VPS41 |
| ENSG00000136518 | ACTL6A |
| ENSG00000100297 | MCM5 |
| ENSG00000165898 | ISCA2 |
| ENSG00000156384 | SFR1 |
| ENSG00000145414 | NAF1 |
| ENSG00000101972 | STAG2 |
| ENSG00000112658 | SRF |
| ENSG00000162736 | NCSTN |
| ENSG00000103266 | STUB1 |
| ENSG00000008018 | PSMB1 |
| ENSG00000149506 | ZP1 |
| ENSG00000111530 | CAND1 |
| ENSG00000027001 | MIPEP |
| ENSG00000152266 | PTH |
| ENSG00000154174 | TOMM70A |
| ENSG00000164045 | CDC25A |
| ENSG00000164758 | MED30 |
| ENSG00000160401 | C9orf117 |
| ENSG00000155959 | VBP1 |
| ENSG00000105409 | ATP1A3 |
| ENSG00000175106 | TVP23C |
| ENSG00000185950 | IRS2 |
| ENSG00000149256 | TENM4 |
| ENSG00000116957 | TBCE |
| ENSG00000154719 | MRPL39 |
| ENSG00000105364 | MRPL4 |
| ENSG00000198218 | QRICH1 |
| ENSG00000013503 | POLR3B |
| ENSG00000126756 | UXT |
| ENSG00000184988 | TMEM106A |
| ENSG00000186432 | KPNA4 |
| ENSG00000156304 | SCAF4 |
| ENSG00000090565 | RAB11FIP3 |
| ENSG00000163508 | EOMES |
| ENSG00000147003 | TMEM27 |
| ENSG00000198730 | CTR9 |
| ENSG00000105321 | CCDC9 |
| ENSG00000120333 | MRPS14 |
| ENSG00000121680 | PEX16 |
| ENSG00000088205 | DDX18 |
| ENSG00000132432 | SEC61G |
| ENSG00000186329 | TMEM212 |
| ENSG00000094804 | CDC6 |
| ENSG00000169084 | DHRSX |
| ENSG00000107618 | RBP3 |
| ENSG00000146426 | TIAM2 |
| ENSG00000198925 | ATG9A |
| ENSG00000168242 | HIST1H2BI |
| ENSG00000254772 | EEF1G |
| ENSG00000090971 | NAT14 |
| ENSG00000144381 | HSPD1 |
| ENSG00000127774 | EMC6 |
| ENSG00000126259 | KIRREL2 |
| ENSG00000111364 | DDX55 |
| ENSG00000100749 | VRK1 |
| ENSG00000159063 | ALG8 |
| ENSG00000163795 | ZNF513 |
| ENSG00000068394 | GPKOW |
| ENSG00000112659 | CUL9 |
| ENSG00000187257 | RSBN1L |
| ENSG00000172167 | MTBP |
| ENSG00000176177 | ENTHD1 |
| ENSG00000166783 | KIAA0430 |
| ENSG00000165006 | UBAP1 |
| ENSG00000188958 | UTS2B |
| ENSG00000136247 | ZDHHC4 |
| ENSG00000196363 | WDR5 |
| ENSG00000116661 | FBXO2 |
| ENSG00000113013 | HSPA9 |
| ENSG00000090061 | CCNK |
| ENSG00000051596 | THOC3 |
| ENSG00000140534 | TICRR |
| ENSG00000100216 | TOMM22 |
| ENSG00000104613 | INTS10 |
| ENSG00000183474 | GTF2H2C |
| ENSG00000159128 | IFNGR2 |
| ENSG00000243725 | TTC4 |
| ENSG00000102898 | NUTF2 |
| ENSG00000170515 | PA2G4 |
| ENSG00000117036 | ETV3 |
| ENSG00000196262 | PPIA |
| ENSG00000153037 | SRP19 |
| ENSG00000135801 | TAF5L |
| ENSG00000119414 | PPP6C |
| ENSG00000141013 | GAS8 |
| ENSG00000113845 | TIMMDC1 |
| ENSG00000175826 | CTDNEP1 |
| ENSG00000117543 | DPH5 |
| ENSG00000204779 | FOXD4L5 |
| ENSG00000112249 | ASCC3 |
| ENSG00000152256 | PDK1 |
| ENSG00000169217 | CD2BP2 |
| ENSG00000166246 | C16orf71 |
| ENSG00000184164 | CRELD2 |
| ENSG00000107960 | OBFC1 |
| ENSG00000102384 | CENPI |
| ENSG00000079785 | DDX1 |
| ENSG00000133858 | ZFC3H1 |
| ENSG00000184110 | EIF3C |
| ENSG00000146700 | SRCRB4D |
| ENSG00000163380 | LMOD3 |
| ENSG00000116273 | PHF13 |
| ENSG00000178229 | ZNF543 |
| ENSG00000109475 | RPL34 |
| ENSG00000156469 | MTERFD1 |
| ENSG00000155827 | RNF20 |
| ENSG00000213741 | RPS29 |
| ENSG00000165792 | METTL17 |
| ENSG00000110844 | PRPF40B |
| ENSG00000100842 | EFS |
| ENSG00000087495 | PHACTR3 |
| ENSG00000126261 | UBA2 |
| ENSG00000136718 | IMP4 |
| ENSG00000091640 | SPAG7 |
| ENSG00000184886 | PIGW |
| ENSG00000184313 | MROH7 |
| ENSG00000163481 | RNF25 |
| ENSG00000137054 | POLR1E |
| ENSG00000213085 | CCDC19 |
| ENSG00000171858 | RPS21 |
| ENSG00000130822 | PNCK |
| ENSG00000145216 | FIP1L1 |
| ENSG00000147130 | ZMYM3 |
| ENSG00000008086 | CDKL5 |
| ENSG00000165282 | PIGO |
| ENSG00000038358 | EDC4 |
| ENSG00000134684 | YARS |
| ENSG00000153832 | FBXO36 |
| ENSG00000140006 | WDR89 |
| ENSG00000104643 | MTMR9 |
| ENSG00000151779 | NBAS |
| ENSG00000077348 | EXOSC5 |
| ENSG00000131043 | AAR2 |
| ENSG00000160193 | WDR4 |
| ENSG00000140691 | ARMC5 |
| ENSG00000141959 | PFKL |
| ENSG00000112053 | SLC26A8 |
| ENSG00000197111 | PCBP2 |
| ENSG00000145191 | EIF2B5 |
| ENSG00000140988 | RPS2 |
| ENSG00000181472 | ZBTB2 |

The gene symbols used in herein (including in Tables 3 and 4) are based on those found in the Human Gene Naming Committee (HGNC) which is searchable on the world-wide web at www.genenames.org. Ensembl IDs are provided for each gene symbol and are searchable world-wide web at www.ensembl.org.

The genes provided in Tables 3 and 4 are non-limiting examples of essential genes. Although additional essential genes will be apparent to the skilled artisan based on the knowledge in the art, the suitability of a particular gene for use according to the present disclosure can be determined, e.g., as discussed herein. For example, in some embodiments, a particular essential gene can be selected by analysis of potential off-target sites elsewhere in the genome. In some embodiments, only essential genes with one or more gRNA target sites that are unique in the human genome are selected for methods described herein. In some embodiments, only essential genes with one or more gRNA target sites that are found in only one other locus in the human genome are selected for methods described herein. In some embodiments, only essential genes with one or more gRNA target sites found in only two other loci in the human genome are selected for methods described herein.

### Gene Product of Interest

The methods, systems and cells of the present disclosure enable the integration of a gene of interest at an essential gene of a cell. The gene of interest can encode any gene product of interest. In certain embodiments, a gene product of interest comprises an antibody, an antigen, an enzyme, a growth factor, a receptor (e.g., cell surface, cytoplasmic, or nuclear), a hormone, a lymphokine, a cytokine, a chemokine, a reporter, a functional fragment of any of the above, or a combination of any of the above.

In some embodiments, sequence for a gene product of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, a degradation signal, and the like.

In some embodiments, an exemplary gene product of interest is one that confers therapeutic value, e.g., a new therapeutic activity to the cell. In some embodiments, exemplary gene products of interest are polypeptides such as a chimeric antigen receptor (CAR) or antigen-binding fragment thereof, a T cell receptor or antigen binding fragment thereof, a non-naturally occurring variant of FcγRIII (CD16), interleukin 15 (IL-15), interleukin 15 receptor (IL-15R) or a variant thereof, interleukin 12 (IL-12), interleukin-12 receptor (IL-12R) or a variant thereof, human leukocyte antigen G (HLA-G), human leukocyte antigen E (HLA-E), leukocyte surface antigen cluster of differentiation CD47 (CD47), or any combination of two or more thereof. It is to be understood that the methods and cells of the present disclosure are not limited to any particular gene product of interest and that the selection of a gene product of interest will depend on the type of cell and ultimate use of the cells.

In some embodiments, a gene product of interest may be a cytokine. In some embodiments, expression of a cytokine from a modified cell generated using a method as described herein allows for localized dosing of the cytokine in vivo (e.g., within a subject in need thereof) and/or avoids a need to systemically administer a high-dose of the cytokine to a subject in need thereof (e.g., a lower dose of the cytokine may be administered). In some embodiments, the risk of dose-limiting toxicities associated with administering a cytokine is reduced while cytokine mediated cell functions are maintained. In some embodiments, to facilitate cell function without the need to additionally administer high-doses of soluble cytokines, a partial or full peptide of one or more of IL2, IL4, IL6, IL7, IL9, IL10, IL11, IL12, IL15, IL18, IL21, IFN-α, IFN-β and/or their respective receptor is introduced to the cell to enable cytokine signaling with or without the expression of the cytokine itself, thereby maintaining or improving cell growth, proliferation, expansion, and/or effector function with reduced risk of cytokine toxicities. In some embodiments, the introduced cytokine and/or its respective native or modified receptor for cytokine signaling are expressed on the cell surface. In some embodiments, the cytokine signaling is constitutively activated. In some embodiments, the activation of the cytokine signaling is inducible. In some embodiments, the activation of the cytokine signaling is transient and/or temporal. In some embodiments, a gene product if interest can be IL2, IL3, IL4, IL6, IL7, IL9, IL10, IL11, IL12, IL13, IL15, IL21, GM-CSF, IFN-a, IFN-b, IFN-g, erythropoietin, and/or the respective cytokine receptor. In some embodiments, a gene product of interest can be CCL3, TNFα, CCL23, IL2RB, IL12RB2, or IRF7.

In some embodiments, a gene product of interest can be a chemokine and/or the respective chemokine receptor. In some embodiments, a chemokine receptor can be, but is not limited to, CCR2, CCR5, CCR8, CX3C1, CX3CR1, CXCR1, CXCR2, CXCR3A, CXCR3B, or CXCR2. In some embodiments, a chemokine can be, but is not limited to, CCL7, CCL19, or CXL14.

As used herein, the term “chimeric antigen receptor” or “CAR” refers to a receptor protein that has been modified to give cells expressing the CAR the new ability to target a specific protein. Within the context of the disclosure, a cell modified to comprise a CAR or an antigen binding fragment may be used for immunotherapy to target and destroy cells associated with a disease or disorder, e.g., cancer cells. In some embodiments, the CAR can bind to any antigen of interest.

CARs of interest can include, but are not limited to, a CAR targeting mesothelin, EGFR, HER2 and/or MICA/B. To date, mesothelin-targeted CAR T-cell therapy has shown early evidence of efficacy in a phase I clinical trial of subjects having mesothelioma, non-small cell lung cancer, and breast cancer (NCT02414269). Similarly, CARs targeting EGFR, HER2 and MICA/B have shown promise in early studies (see, e.g., Li et al. (2018), Cell Death & Disease, 9(177); Han et al. (2018) Am. J. Cancer Res., 8(1):106-119; and Demoulin 2017) Future Oncology, 13(8); the entire contents of each of which are expressly incorporated herein by reference in their entireties).

CARs are well-known to those of ordinary skill in the art and include those described in, for example: WO13/063419 (mesothelin), WO15/164594 (EGFR), WO13/063419 (HER2), WO16/154585 (MICA and MICB), the entire contents of each of which are expressly incorporated herein by reference in their entireties. In some embodiments, a gene product of interest is any suitable CAR, NK cell specific CAR (NK-CAR), T cell specific CAR, or other binder that targets a cell, e.g., an NK cell, to a target cell, e.g., a cell associated with a disease or disorder, may be expressed in the modified cells provided herein. Exemplary CARs, and binders, include, but are not limited to, bi-specific antigen binding CARs, switchable CARs, dimerizable CARs, split CARs, multi-chain CARs, inducible CARs, CARs and binders that bind BCMA, androgen receptor, PSMA, PSCA, Muc1, HPV viral peptides (i.e., E7), EBV viral peptides, WT1, CEA, EGFR, EGFRVIII, IL 13Rα2, GD2, CA125, EpCAM, Muc16, carbonic anhydrase IX (CAIX), CCR1, CCR4, carcinoembryonic antigen (CEA), CD3, CD5, CD7, CD10, CD19, CD20, CD22, CD23, CD24, CD26, CD30, CD33, CD34, CD35, CD38⁺CD41, CD44, CD44V6, CD49f, CD56, CD70, CD92, CD99, CD123, CD133, CD135, CD148, CD150, CD261, CD362, CLEC12A, MDM2, CYP1B, livin, cyclin 1, NKp30, NKp46, DNAM1, NKp44, CA9, PD1, PDL1, an antigen of cytomegalovirus (CMV), epithelial glycoprotein-40 (EGP-40), GPRC5D, receptor tyrosine kinases erb-B2,3,4, EGFIR, ERBB folate binding protein (FBP), fetal acetylcholine receptor (AChR), folate receptor-a, ganglioside G3 (GD3) human Epidermal Growth Factor Receptor 2 (HER-2), human telomerase reverse transcriptase (hTERT), ICAM-1, Integrin B7, Interleukin-13 receptor subunit alpha-2 (IL-13Rα2), K-light chain, kinase insert domain receptor (KDR), Lewis A (CA19.9), Lewis Y (Le Y), L1 cell adhesion molecule (LI-CAM), LILRB2, melanoma antigen family A 1 (MAGE-A1), MICA/B, Mucin 16 (Muc-16), NKCSI, NKG2D ligands, c-Met, cancer-testis antigen NYESO-1, oncofetal antigen (h5T4), PRAME, prostate stem cell antigen (PSCA), PRAME prostate-specific membrane antigen (PSMA), tumor-associated glycoprotein 72 (TAG-72), TIM-3, TRBCI, TRBC2, vascular endothelial growth factor R2 (VEGF-R2), Wilms tumor protein (WT-1), a pathogen antigen, or any suitable combination thereof. Additional suitable CARs and binders for use in the modified cells provided herein will be apparent to those of skill in the art based on the present disclosure and the general knowledge in the art. Such additional suitable CARs include those described in FIG. 3 of Davies and Maher, Adoptive T-cell Immunotherapy of Cancer Using Chimeric Antigen Receptor-Grafted T Cells, Archivum Immunologiae et Therapiae Experimentalis 58(3): 165-78 (2010), the entire contents of which are incorporated herein by reference. Additional CARs suitable for methods described herein include: CD171-specific CARs (Park et al., Mol Ther (2007) 15(4):825-833), EGFRvIII-specific CARs (Morgan et al, Hum Gene Ther (2012) 23(10): 1043-1053), EGF-R-specific CARs (Kobold et al, J Natl Cancer Inst (2014) 10⁷(1):364), carbonic anhydrase K-specific CARs (Lamers et al., Biochem Soc Trans (2016) 44(3): 951-959), FR-a-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10): 1779-1787; Luo et al., Cell Res (2016) 26(7):850-853; Morgan et al., Mol Ther (2010) 18(4):843-85 1; Grada et al., Mol Ther Nucleic Acids (2013) 9(2):32), CEA-specific CARs (Katz et al., Clin Cancer Res (2015) 21 (14):3149-3159), IL13Ra2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5): 1059-1070), VEGF-R-specific CARs (Chinnasamy et al., Cancer Res (2016) 22(2):436-447), FAP-specific CARs (Wang et al., Cancer Immunol Res (2014) 2(2): 154-166), MSLN-specific CARs (Moon et al., Clin Cancer Res (2011) 17(14):4719-30), CD19-specific CARs (Axicabtagene ciloleucel (Yescarta®) and Tisagenlecleucel (Kymriah®). See also, Li et al., J Hematol and Oncol (2018) 11(22), reviewing clinical trials of tumor-specific CARs. In some embodiments, a CAR is an anti-EGFR CAR. In some embodiments, a CAR is an anti-CD19 CAR. In some embodiments, a CAR is an anti-BCMA CAR. In some embodiments, a CAR is an anti-CD7 CAR.

As used herein, the term “CD16” refers to a receptor (FcγRIII) for the Fc portion of immunoglobulin G, and it is involved in the removal of antigen-antibody complexes from the circulation, as well as other antibody-dependent responses. In some embodiments, a CD16 protein is an hCD16 variant. In some embodiments an hCD16 variant is a high affinity F158V variant.

In some embodiments, a gene product of interest comprises a high affinity non-cleavable CD16 (hnCD16) or a variant thereof. In some embodiments, a high affinity non-cleavable CD16 or a variant thereof comprises at least any one of the followings: (a) F176V and S197P in ectodomain domain of CD16 (see e.g., Jing et al., Identification of an ADAM17 Cleavage Region in Human CD16 (FcγRIII) and the Engineering of a Non-Cleavable Version of the Receptor in NK Cells; PLOS One, 2015); (b) a full or partial ectodomain originated from CD64; (c) a non-native (or non-CD16) transmembrane domain; (d) a non-native (or non-CD16) intracellular domain; (e) a non-native (or nonCD16) signaling domain; (f) a non-native stimulatory domain; and (g) transmembrane, signaling, and stimulatory domains that are not originated from CD16, and are originated from a same or different polypeptide. In some embodiments, the non-native transmembrane domain is derived from CD3D, CD3E, CD3G, CD3s, CD4, CD5, CD5a, CD5b, CD27, CD2S, CD40, CDS4, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor (TCR) polypeptide. In some embodiments, the non-native stimulatory domain is derived from CD27, CD2S, 4-1BB, OX40, ICOS, PD-1, LAG-3, 2B4, BTLA, DAP10, DAP12, CTLA-4, or NKG2D polypeptide. In some other embodiments, the non-native signaling domain is derived from CD3s, 2B4, DAP10, DAP12, DNAMI, CD137 (41BB), IL21, IL7, IL12, IL15, NKp30, NKp44, NKp46, NKG2C, or NKG2D polypeptide. In some particular embodiments of a hnCD16 variant, the non-native transmembrane domain is derived from NKG2D, the non-native stimulatory domain is derived from 2B4, and the non-native signaling domain is derived from CD3s. In some embodiments, a gene product of interest comprises a high affinity cleavable CD16 (hnCD16) or a variant thereof. In some embodiments, a high affinity cleavable CD16 or a variant thereof comprises at least F176V. In some embodiments, a high affinity cleavable CD16 or a variant thereof does not comprise an S197P amino acid substitution.

As used herein, the term “IL-15/IL15RA” or “Interleukin-15” (IL-15) refers to a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells. IL-15 Receptor alpha (IL15RA) specifically binds IL-15 with very high affinity, and is capable of binding IL-15 independently of other subunits (see e.g., Mishra et al., Molecular pathways: Interleukin-15 signaling in health and in cancer, Clinical Cancer Research, 2014). It is suggested that this property allows IL-15 to be produced by one cell, endocytosed by another cell, and then presented to a third party cell. IL15RA is reported to enhance cell proliferation and expression of apoptosis inhibitor BCL2L1/BCL2-XL and BCL2. Exemplary sequences of IL-15 are provided in NG_029605.2, and exemplary sequences of IL-15RA are provided in NM_002189.4. In some embodiments, the IL-15R variant is a constitutively active IL-15R variant. In some embodiments, the constitutively active IL-15R variant is a fusion between IL-15R and an IL-15R agonist, e.g., an IL-15 protein or IL-15R-binding fragment thereof. In some embodiments, the IL-15R agonist is IL-15, or an IL-15R-binding variant thereof. Exemplary suitable IL-15R variants include, without limitation, those described, e.g., in Mortier E et al, 2006; The Journal of Biological Chemistry 2006 281: 1612-1619; or in Bessard-A et al., Mol Cancer Ther. 2009 September; 8(9):2736-45, the entire contents of each of which are incorporated by reference herein. In some embodiments, membrane bound trans-presentation of IL-15 is a more potent activation pathway than soluble IL-15 (see e.g., Imamura et al., Autonomous growth and increased cytotoxicity of natural killer cells expressing membrane-bound interleukin-15, Blood, 2014). In some embodiments, IL-15R expression comprises: IL15 and IL 15Ra expression using a self-cleaving peptide; a fusion protein of IL 15 and IL15Ra; an IL15/IL15Ra fusion protein with intracellular domain of IL 15Ra truncated; a fusion protein of IL 15 and membrane bound Sushi domain of IL 15Ra; a fusion protein of IL15 and IL15Rβ; a fusion protein of IL 15 and common receptor γC, wherein the common receptor γC is native or modified; and/or a homodimer of IL15Rβ.

As used herein, the term “IL-12” refers to interleukin-12, a cytokine that acts on T and natural killer cells. In some embodiments, a genetically engineered stem cell and/or progeny cell comprises a genetic modification that leads to expression of one or more of an interleukin 12 (IL12) pathway agonist, e.g., IL-12, interleukin 12 receptor (IL-12R) or a variant thereof (e.g., a constitutively active variant of IL-12R, e.g., an IL-12R fused to an IL-12R agonist (IL-12RA).

In some embodiments, the gene product of interest comprises a protein or polypeptide whose expression within a cell, e.g., a cell modified as described herein, enables the cell to inhibit or evade immune rejection after transplant or engraftment into a subject. In some embodiments, the gene product of interest is HLA-E, HLA-G, CTL4, CD47, or an associated ligand.

In some embodiments, the gene product of interest is a T cell receptor (TCR) or an antigen-binding fragment thereof, e.g., a recombinant TCR. In some embodiments, the recombinant TCR can bind to an antigen of interest, e.g., an antigen selected from, but not limited to, CD279, CD2, CD95, CD152, CD223CD272, TIM3, KIR, A2aR, SIRPa, CD200, CD200R, CD300, LPA5, NY-ESO, PD1, PDL1, or MAGE-A3/A6. In some embodiments, the TCR or antigen-binding fragment thereof can bind to a viral antigen, e.g., an antigen from hepatitis A, hepatitis B, hepatitis C (HCV), human papilloma virus (HPV) (e.g., HPV-16 (such as HPV-16 E6 or HPV-16 E7), HPV-18, HPV-31, HPV-33, or HPV-35), Epstein-Barr virus (EBV), human herpes virus 8 (HHV-8), human T-cell leukemia virus01 (HTLV-1), human T-cell leukemia virus-2 (HTLV-2) or a cytomegalovirus (CMV).

In some embodiments, the gene product of interest comprises a single-chain variable fragment that can bind to CD47, PD1, CTLA4, CD28, OX40, 4-1BB, and ligands thereof.

As used herein, the term “HLA-G” refers to the HLA non-classical class I heavy chain paralogues. This class I molecule is a heterodimer consisting of a heavy chain and a light chain (beta-2 microglobulin). The heavy chain is anchored in the membrane. HLA-G is expressed on fetal derived placental cells. HLA-G is a ligand for NK cell inhibitory receptor KIR2DL4, and therefore expression of this HLA by the trophoblast defends it against NK cell-mediated death. See e.g., Favier et al., Tolerogenic Function of Dimeric Forms of HLA-G Recombinant Proteins: A Comparative Study In Vivo PLOS One 2011, the entire contents of which are incorporated herein by reference. An exemplary sequence of HLA-G is set forth as NG_029039.1.

As used herein, the term “HLA-E” refers to the HLA class I histocompatibility antigen, alpha chain E, also sometimes referred to as MHC class I antigen E. The HLA-E protein in humans is encoded by the HLA-E gene. The human HLA-E is a non-classical MHC class I molecule that is characterized by a limited polymorphism and a lower cell surface expression than its classical paralogues. This class I molecule is a heterodimer consisting of a heavy chain and a light chain (beta-2 microglobulin). The heavy chain is anchored in the membrane. HLA-E binds a restricted subset of peptides derived from the leader peptides of other class I molecules. HLA-E expressing cells escape allogeneic responses and lysis by NK cells. See e.g., Geornalusse-G et al., Nature Biotechnology 2017 35(8), the entire contents of which are incorporated herein by reference. Exemplary sequences of the HLA-E protein are provided in NM_005516.6.

As used herein, the term “CD47,” also sometimes referred to as “integrin associated protein” (IAP), refers to a transmembrane protein that in humans is encoded by the CD47 gene. CD47 belongs to the immunoglobulin superfamily, partners with membrane integrins, and also binds the ligands thrombospondin-1 (TSP-1) and signal-regulatory protein alpha (SIRPa). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

In some embodiments, a gene product of interest comprises a chimeric switch receptor (see e.g., WO2018094244A1—TGFBeta Signal Converter; Ankri et al., Human T cells Engineered to express a programmed death 1/28 costimulatory retargeting molecule display enhanced antitumor activity, The Journal of Immunology, Oct. 15, 2013, 191; Roth et al., Pooled knockin targeting for genome engineering of cellular immunotherapies, Cell. 2020 Apr. 30; 181(3):728-744.e21; and Boyerinas et al., A Novel TGF-β2/Interleukin Receptor Signal Conversion Platform That Protects CAR/TCR T Cells from TGF-β2-Mediated Immune Suppression and Induces T Cell Supportive Signaling Networks, Blood, 2017). In some embodiments, chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, a chimeric switch receptor comprises an extracellular domain of an inhibitory cell-surface receptor fused to an intracellular domain that leads to the transmission of an activating signal rather than the inhibitory signal normally transduced by the inhibitory cell-surface receptor. In some embodiments, extracellular domains derived from cell-surface receptors known to inhibit immune effector cell activation can be fused to activating intracellular domains. In such an embodiment, engagement of the corresponding ligand may then activate signaling cascades that increase, rather than inhibit, the activation of the immune effector cell. For example, in some embodiments, a gene product of interest is a PD1-CD28 switch receptor, wherein the extracellular domain of PD1 is fused to the intracellular signaling domain of CD28 (See e.g., Liu et al., Cancer Res 76:6 (2016), 1578-1590 and Moon et al., Molecular Therapy 22 (2014), S201). In some embodiments, encoding gene product of interest is or comprises the extracellular domain of CD200R and the intracellular signaling domain of CD28 (See Oda et al., Blood 130:22 (2017), 2410-2419).

In some embodiments, a gene product of interest is a reporter gene (e.g., GFP, mCherry, etc.). In some embodiments, a reporter gene is utilized to confirm the suitability of a knock-in cassette's expression capacity. In certain embodiments, a gene product of interest may be a colored or fluorescent protein such as: blue/UV proteins, e.g. TagBFP, mTagBFP2, Azurite, EBFP2, mKalamal, Sirius, Sapphire, T-Sapphire; cyan proteins, e.g. ECFP, Cerulean, SCFP3A, m Turquoise, mTurquoise2, monomeric Midoriishi-Cyan, TagCFP, mTFPl; green proteins, e.g. EGFP, Emerald, Superfolder GFP, Monomeric Azami Green, TagGFP2, mUKG, m Wasabi, Clover, mNeonGreen; yellow proteins, e.g. EYFP, Citrine, Venus, SYFP2, TagYFP; orange proteins, e.g. Monomeric Kusabira-Orange, mKOK, mK02, mOrange, m0range2; red proteins, e.g. mRaspberry, mStrawberry, mTangerine, tdTomato, TagRFP, TagRFP-T, mApple, mRuby, mRuby2; far-red proteins, e.g. mPlum, HcRed-Tandem, mKate2, mNeptune, NirFP; near-IR proteins, e.g. TagRFP657, IFPl.4, iRFP; long stokes shift proteins, e.g. mKeima Red, LSS-mKatel, LSS-mKate2, mBeRFP; photoactivatible proteins, e.g. PA-GFP, PAmCherryl, PATagRFP; photoconvertible proteins, e.g. Kaede (green), Kaede (red), KikGRI (green), KikGR1 (red), PS-CFP2, PS-CFP2, mEos2 (green), mEos2 (red), mEos3.2 (green), mEos3.2 (red), PSmOrange, PSmOrange, photoswitchable proteins, e.g. Dronpa, and combinations thereof.

In some embodiments, a gene of interest provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al. (2012) Cell 126(5): 995-1004, which is incorporated in its entirety herein by reference). In some embodiments, a destabilizing sequence is a DHFR sequence, and a stabilizing ligand is trimethoprim (TMP) (Iwamoto et al. (2010) Chem Biol 17:981-988, which is incorporated in its entirety herein by reference). In some embodiments, a destabilizing domain is small molecule-assisted shutoff (SMASh), where a constitutive degron with a protease and its corresponding cleavage site derived from hepatitis C virus are combined. In some embodiments, a destabilizing domain comprises a HaloTag system, dTag system, and/or nanobody (see e.g., Luh et al., Prey for the proteasome: targeted protein degradation—a medicinal chemist's perspective; Angewandte Chemie, 2020).

In some embodiments, a destabilizing sequence can be used to temporally control a cell modified as described herein.

In some embodiments, a gene product of interest may be a suicide gene, (see e.g., Zarogoulidis et al., Suicide Gene Therapy for Cancer—Current Strategies; J Genet Syndr Gene Ther. 2013). In some embodiments, a suicide gene can use a gene-directed enzyme prodrug therapy (GDEPT) approach, a dimerization inducing approach, and/or therapeutic monoclonal antibody mediated approach. In some embodiments, a suicide gene is biologically inert, has an adequate bio-availability profile, an adequate bio-distribution profile, and can be characterized by intrinsic acceptable and/or absence of toxicity. In some embodiments, a suicide gene codes for a protein able to convert, at a cellular level, a non-toxic prodrug into a toxic product. In some embodiments, a suicide gene may improve the safety profile of a cell described herein (see e.g., Greco et al., Improving the safety of cell therapy with the TK-suicide gene; Front Pharmacology. 2015; Jones et al., Improving the safety of cell therapy products by suicide gene transfer; Frontiers Pharmacology, 2014). In some embodiments, a suicide gene is a herpes simplex virus thymidine kinase (HSV-TK). In some embodiments, a suicide gene is a cytosine deaminase (CD). In some embodiments, a suicide gene is an apoptotic gene (e.g., a caspase). In some embodiments, a suicide gene is dimerization inducing, e.g., comprising an inducible FAS (iFAS) or inducible Caspase9 (iCasp9)/AP1903 system. In some embodiments, a suicide gene is a CD20 antigen, and cells expressing such an antigen can be eliminated by clinical-grade anti-CD20 antibody administration. In some embodiments, a suicide gene is a truncated human EGFR polypeptide (huEGFRt) which confers sensitivity to a pharmaceutical-grade anti-EGFR monoclonal antibody, e.g., cetuximab. In some embodiments a suicide gene is a c-myc tag, which confers sensitivity to pharmaceutical-grade anti-cmyc antibodies.

Exemplary DHFR destabilizing amino acid sequence
SEQ ID NO: 161
MISLIAALAVDYVIGMENAMPWNLPADLAWFKRNTLNKPVIMGRHTWESIGRPLPGRKNIILSS
QPSTDDRVTWVKSVDEAIAACGDVPEIMVIGGGRVIEQFLPKAQKLYLTHIDAEVEGDTHEPDY
EPDDWESVESEFHDADAQNSHSYCFEILERR
Exemplary DHFR destabilizing nucleotide sequence
SEQ ID NO: 162
GGTACCATCAGTCTGATTGCGGCGTTAGCGGTAGATTACGTTATCGGCATGGAAAACGCCATGC
CGTGGAACCTGCCTGCCGATCTCGCCTGGTTTAAACGCAACACCTTAAATAAACCCGTGATTAT
GGGCCGCCATACCTGGGAATCAATCGGTCGTCCGTTGCCAGGACGCAAAAATATTATCCTCAGC
AGTCAACCGAGTACGGACGATCGCGTAACGTGGGTGAAGTCGGTGGATGAAGCCATCGCGGCGT
GTGGTGACGTACCAGAAATCATGGTGATTGGCGGCGGTCGCGTTATTGAACAGTTCTTGCCAAA
AGCGCAAAAACTGTATCTGACGCATATCGACGCAGAAGTGGAAGGCGACACCCATTTCCCGGAT
TACGAGCCGGATGACTGGGAATCGGTATTCAGCGAATTCCACGATGCTGATGCGCAGAACTCTC
ACAGCTATTGCTTTGAGATTCTGGAGCGGCGATAA
Exemplary destabilizing domain
SEQ ID NO: 163
ATCAGTCTGATTGCGGCGTTAGCGGTAGATTACGTTATCGGCATGGAAAACGCCATGCCGTGGA
ACCTGCCTGCCGATCTCGCCTGGTTTAAACGCAACACCTTAAATAAACCCGTGATTATGGGCCG
CCATACCTGGGAATCAATCGGTCGTCCGTTGCCAGGACGCAAAAATATTATCCTCAGCAGTCAA
CCGAGTACGGACGATCGCGTAACGTGGGTGAAGTCGGTGGATGAAGCCATCGCGGCGTGTGGTG
ACGTACCAGAAATCATGGTGATTGGCGGCGGTCGCGTTATTGAACAGTTCTTGCCAAAAGCGCA
AAAACTGTATCTGACGCATATCGACGCAGAAGTGGAAGGCGACACCCATTTCCCGGATTACGAG
CCGGATGACTGGGAATCGGTATTCAGCGAATTCCACGATGCTGATGCGCAGAACTCTCACAGCT
ATTGCTTTGAGATTCTGGAGCGGCGA
Exemplary FKBP12 destabilizing peptide amino acid sequence
SEQ ID NO: 164
MGVEKQVIRPGNGPKPAPGQTVTVHCTGFGKDGDLSQKFWSTKDEGQKPFSFQIGKGAVIKGWD
EGVIGMQIGEVARLRCSSDYAYGAGGFPAWGIQPNSVLDFEIEVLSVQ

In some embodiments, a coding sequence for a single gene product of interest may be included in a knock-in cassette. In some embodiments, coding sequences for two gene products of interest may be included in a single knock-in cassette; in some embodiments, this may be referred to as a bicistronic or multicistronic construct. In some embodiments, coding sequences for more than two gene products of interest may be included in a single knock-in cassette; in some embodiments, this may be referred to as a multicistronic construct. In some embodiments, when more than one coding sequence for more than one gene product of interest is included in a knock-in cassette, these sequences may have a linker sequence connecting them. Linker sequences are generally known in the art, an exemplary linker sequence is identified in SEQ ID NO: 164000. In some embodiments, where more than one coding sequence for more than one gene product of interest is included in a knock-in cassette, these sequences may be connected by a linker sequence, an IRES, and/or 2A element.

In some embodiments, a polynucleotide encoding a gene product of interest comprises or consists of the sequence of any one of SEQ ID NOs: 162-163, 165-182, or 164000. In some embodiments, a polynucleotide encoding a gene product of interest comprises or consists of a sequence that is at least 85%, 90%, 95%, 96%, 97%, 98%, 99%, 99.1%, 99.2%, 99.3%, 99.4%, 99.5%, 99.6%, 99.7%, 99.8%, or 99.9% identical to any one of SEQ ID NOs: 162-163, 165-182, or 164000. In some embodiments, a polynucleotide encoding a gene product of interest comprises or consists of a functional variant of any one of SEQ ID NOs: 162-163, 165-182, or 164000. In some embodiments, a polynucleotide encoding a gene product of interest comprises or consists of a nucleotide sequence having 1, 2, 3, 4, 5, 6, 7, 8, 9, or 10 mutations (e.g., substitutions, insertions, and/or deletions) relative to any one of SEQ ID NOs: 162-163, 165-182, or 164000.

exemplary linker sequence
SEQ ID NO: 164000
TCTGGCGGAGGAAGCGGAGGCGGAGGATCTGGTGGTGGTGGATCTGGCGGCGGTGGTAGTGGCG
GAGGTTCTCTGCAA
exemplary CD16 knock-in cassette sequence
SEQ ID NO: 165
ATGTGGCAACTGCTGCTGCCTACAGCTCTGCTGCTTCTGGTGTCTGCCGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGTAA
exemplary CD16 knock-in cassette sequence
SEQ ID NO: 166
ATGTGGCAGCTGTTGCTGCCGACAGCCCTCCTGTTGCTGGTCTCCGCTGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAG
exemplary CD47 knock-in cassette sequence
SEQ ID NO: 167
ATGTGGCCCCTGGTAGCGGCGCTGTTGCTGGGCTCGGCGTGCTGCGGATCAGCTCAGCTACTAT
TTAATAAAACAAAATCTGTAGAATTCACGTTTTGTAATGACACTGTCGTCATTCCATGCTTTGT
TACTAATATGGAGGCACAAAACACTACTGAAGTATACGTAAAGTGGAAATTTAAAGGAAGAGAT
ATTTACACCTTTGATGGAGCTCTAAACAAGTCCACTGTCCCCACTGACTTTAGTAGTGCAAAAA
TTGAAGTCTCACAATTACTAAAAGGAGATGCCTCTTTGAAGATGGATAAGAGTGATGCTGTCTC
ACACACAGGAAACTACACTTGTGAAGTAACAGAATTAACCAGAGAAGGTGAAACGATCATCGAG
CTAAAATATCGTGTTGTTTCATGGTTTTCTCCAAATGAAAATATTCTTATTGTTATTTTCCCAA
TTTTTGCTATACTCCTGTTCTGGGGACAGTTTGGTATTAAAACACTTAAATATAGATCCGGTGG
TATGGATGAGAAAACAATTGCTTTACTTGTTGCTGGACTAGTGATCACTGTCATTGTCATTGTT
GGAGCCATTCTTTTCGTCCCAGGTGAATATTCATTAAAGAATGCTACTGGCCTTGGTTTAATTG
TGACTTCTACAGGGATATTAATATTACTTCACTACTATGTGTTTAGTACAGCGATTGGATTAAC
CTCCTTCGTCATTGCCATATTGGTTATTCAGGTGATAGCCTATATCCTCGCTGTGGTTGGACTG
AGTCTCTGTATTGCGGCGTGTATACCAATGCATGGCCCTCTTCTGATTTCAGGTTTGAGTATCT
TAGCTCTAGCACAATTACTTGGACTAGTTTATATGAAATTTGTGGCTTCCAATCAGAAGACTAT
ACAACCTCCTAGGAAAGCTGTAGAGGAACCCCTTAATGCATTCAAAGAATCAAAAGGAATGATG
AATGATGAATGA
exemplary IL 15 knock-in cassette sequence
SEQ ID NO: 168
AATTGGGTCAACGTGATCAGCGACCTGAAGAAGATCGAGGACCTGATCCAGAGCATGCACATCG
ACGCCACACTGTACACCGAGTCCGATGTGCACCCTAGCTGCAAAGTGACCGCCATGAAGTGCTT
TCTGCTGGAACTGCAAGTGATCAGCCTGGAAAGCGGCGACGCCAGCATCCACGATACCGTGGAA
AACCTGATCATCCTGGCCAACAACAGCCTGAGCAGCAACGGCAATGTGACCGAGAGCGGCTGCA
AAGAGTGCGAGGAACTGGAAGAGAAGAACATCAAAGAGTTCCTCCAGAGCTTCGTCCACATCGT
GCAGATGTTCATCAACACCAGC
exemplary IgE-IL15 knock-in cassette sequence
SEQ ID NO: 169
ATGGATTGGACCTGGATCCTGTTTCTGGTGGCCGCTGCCACAAGAGTGCACAGCAATTGGGTCA
ACGTGATCAGCGACCTGAAGAAGATCGAGGACCTGATCCAGAGCATGCACATCGACGCCACACT
GTACACCGAGTCCGATGTGCACCCTAGCTGCAAAGTGACCGCCATGAAGTGCTTTCTGCTGGAA
CTGCAAGTGATCAGCCTGGAAAGCGGCGACGCCAGCATCCACGATACCGTGGAAAACCTGATCA
TCCTGGCCAACAACAGCCTGAGCAGCAACGGCAATGTGACCGAGAGCGGCTGCAAAGAGTGCGA
GGAACTGGAAGAGAAGAACATCAAAGAGTTCCTCCAGAGCTTCGTCCACATCGTGCAGATGTTC
ATCAACACCAGC
exemplary IgE-IL15 pro-peptide cargo sequence
SEQ ID NO: 170
ATGGACTGGACCTGGATTCTGTTCCTGGTCGCGGCTGCAACGCGAGTCCATAGCGGTATCCATG
TTTTTATTCTTGGGTGTTTTTCTGCTGGGCTGCCTAAGACCGAGGCCAACTGGGTAAATGTCAT
CAGTGACCTCAAGAAAATAGAAGACCTTATACAAAGCATGCACATTGATGCTACTCTCTACACT
GAGTCAGATGTACATCCCTCATGCAAAGTGACGGCCATGAAATGTTTCCTCCTCGAACTTCAAG
TCATATCTCTGGAAAGTGGCGACGCGTCCATCCACGACACGGTCGAAAACCTGATAATACTCGC
TAATAATAGTCTCTCTTCAAATGGTAACGTAACCGAGTCAGGTTGCAAAGAGTGCGAAGAGTTG
GAAGAAAAAAACATAAAGGAGTTCCTGCAAAGTTTCGTGCACATTGTGCAGATGTTCATTAATA
CCTCT
exemplary IL15Rα cargo sequence
SEQ ID NO: 171
ATCACCTGTCCTCCACCTATGAGCGTGGAACACGCCGACATCTGGGTCAAGAGCTACAGCCTGT
ACAGCAGAGAGCGGTACATCTGCAACAGCGGCTTCAAGAGAAAGGCCGGCACAAGCAGCCTGAC
CGAGTGTGTGCTGAACAAGGCCACAAACGTGGCCCACTGGACCACACCTAGCCTGAAGTGCATC
AGAGATCCCGCTCTGGTTCATCAGAGGCCTGCCCCTCCATCTACAGTGACAACAGCTGGCGTGA
CCCCTCAGCCTGAGTCTCTGTCTCCATCTGGAAAAGAGCCTGCCGCCAGCTCTCCCAGCTCTAA
CAATACTGCTGCCACCACAGCCGCTATCGTGCCTGGATCTCAGCTGATGCCTAGCAAGAGCCCT
AGCACCGGCACAACAGAGATCAGCTCTCACGAGAGCAGCCACGGAACACCTTCTCAGACCACCG
CCAAGAATTGGGAGCTGACAGCCTCTGCCTCTCATCAGCCACCTGGCGTGTACCCACAGGGCCA
CTCTGATACAACAGTGGCCATCAGCACCAGCACCGTTCTGCTGTGTGGCCTGTCTGCTGTTAGC
CTGCTGGCCTGCTACCTGAAGTCTAGACAGACACCTCCTCTGGCCAGCGTGGAAATGGAAGCCA
TGGAAGCTCTGCCTGTCACATGGGGCACCAGCAGCAGAGATGAGGACCTCGAGAATTGCAGCCA
CCACCTG
exemplary mbIL-15 cargo sequence
SEQ ID NO: 172
ATGGATTGGACCTGGATCCTGTTTCTGGTGGCCGCTGCCACAAGAGTGCACAGCAATTGGGTCA
ACGTGATCAGCGACCTGAAGAAGATCGAGGACCTGATCCAGAGCATGCACATCGACGCCACACT
GTACACCGAGTCCGATGTGCACCCTAGCTGCAAAGTGACCGCCATGAAGTGCTTTCTGCTGGAA
CTGCAAGTGATCAGCCTGGAAAGCGGCGACGCCAGCATCCACGATACCGTGGAAAACCTGATCA
TCCTGGCCAACAACAGCCTGAGCAGCAACGGCAATGTGACCGAGAGCGGCTGCAAAGAGTGCGA
GGAACTGGAAGAGAAGAACATCAAAGAGTTCCTCCAGAGCTTCGTCCACATCGTGCAGATGTTC
ATCAACACCAGCTCTGGCGGAGGAAGCGGAGGCGGAGGATCTGGTGGTGGTGGATCTGGCGGCG
GTGGTAGTGGCGGAGGTTCTCTGCAAATCACCTGTCCTCCACCTATGAGCGTGGAACACGCCGA
CATCTGGGTCAAGAGCTACAGCCTGTACAGCAGAGAGCGGTACATCTGCAACAGCGGCTTCAAG
AGAAAGGCCGGCACAAGCAGCCTGACCGAGTGTGTGCTGAACAAGGCCACAAACGTGGCCCACT
GGACCACACCTAGCCTGAAGTGCATCAGAGATCCCGCTCTGGTTCATCAGAGGCCTGCCCCTCC
ATCTACAGTGACAACAGCTGGCGTGACCCCTCAGCCTGAGTCTCTGTCTCCATCTGGAAAAGAG
CCTGCCGCCAGCTCTCCCAGCTCTAACAATACTGCTGCCACCACAGCCGCTATCGTGCCTGGAT
CTCAGCTGATGCCTAGCAAGAGCCCTAGCACCGGCACAACAGAGATCAGCTCTCACGAGAGCAG
CCACGGAACACCTTCTCAGACCACCGCCAAGAATTGGGAGCTGACAGCCTCTGCCTCTCATCAG
CCACCTGGCGTGTACCCACAGGGCCACTCTGATACAACAGTGGCCATCAGCACCAGCACCGTTC
TGCTGTGTGGCCTGTCTGCTGTTAGCCTGCTGGCCTGCTACCTGAAGTCTAGACAGACACCTCC
TCTGGCCAGCGTGGAAATGGAAGCCATGGAAGCTCTGCCTGTCACATGGGGCACCAGCAGCAGA
GATGAGGACCTCGAGAATTGCAGCCACCACCTG
exemplary mbIL-15 cargo sequence
SEQ ID NO: 173
ATGGACTGGACCTGGATTCTGTTCCTGGTCGCGGCTGCAACGCGAGTCCATAGCGGTATCCATG
TTTTTATTCTTGGGTGTTTTTCTGCTGGGCTGCCTAAGACCGAGGCCAACTGGGTAAATGTCAT
CAGTGACCTCAAGAAAATAGAAGACCTTATACAAAGCATGCACATTGATGCTACTCTCTACACT
GAGTCAGATGTACATCCCTCATGCAAAGTGACGGCCATGAAATGTTTCCTCCTCGAACTTCAAG
TCATATCTCTGGAAAGTGGCGACGCGTCCATCCACGACACGGTCGAAAACCTGATAATACTCGC
TAATAATAGTCTCTCTTCAAATGGTAACGTAACCGAGTCAGGTTGCAAAGAGTGCGAAGAGTTG
GAAGAAAAAAACATAAAGGAGTTCCTGCAAAGTTTCGTGCACATTGTGCAGATGTTCATTAATA
CCTCTAGCGGCGGAGGATCAGGTGGCGGTGGAAGCGGAGGTGGAGGCTCCGGTGGAGGAGGTAG
TGGCGGAGGTTCTCTTCAAATAACTTGTCCTCCACCGATGTCCGTAGAACATGCGGATATTTGG
GTAAAATCCTATAGCTTGTACAGCCGAGAGCGGTATATCTGCAACAGCGGCTTCAAGCGGAAGG
CCGGCACAAGCAGCCTGACCGAGTGCGTGCTGAACAAGGCCACCAACGTGGCCCACTGGACCAC
CCCTAGCCTGAAGTGCATCAGAGATCCCGCCCTGGTGCATCAGCGGCCTGCCCCTCCAAGCACA
GTGACAACAGCTGGCGTGACCCCCCAGCCTGAGAGCCTGAGCCCTTCTGGAAAAGAGCCTGCCG
CCAGCAGCCCCAGCAGCAACAATACTGCCGCCACCACAGCCGCCATCGTGCCTGGATCTCAGCT
GATGCCCAGCAAGAGCCCTAGCACCGGCACCACCGAGATCAGCAGCCACGAGTCTAGCCACGGC
ACCCCATCTCAGACCACCGCCAAGAACTGGGAGCTGACAGCCAGCGCCTCTCACCAGCCTCCAG
GCGTGTACCCTCAGGGCCACAGCGATACCACAGTGGCCATCAGCACCTCCACCGTGCTGCTGTG
TGGACTGAGCGCCGTGTCACTGCTGGCCTGCTACCTGAAGTCCAGACAGACCCCTCCACTGGCC
AGCGTGGAAATGGAAGCCATGGAAGCACTGCCCGTGACCTGGGGCACCAGCTCCAGAGATGAGG
ATCTGGAAAACTGCTCCCACCACCTG
exemplary multicistronic CD16, mbIL-15 cargo sequence
SEQ ID NO: 174
ATGTGGCAGCTGTTGCTGCCGACAGCCCTCCTGTTGCTGGTCTCCGCTGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGGGAAGC
GGAGCCACAAACTTCTCTCTGCTGAAGCAGGCAGGAGATGTTGAAGAAAACCCTGGACCTATGG
ATTGGACCTGGATCCTGTTTCTGGTGGCCGCTGCCACAAGAGTGCACAGCAATTGGGTCAACGT
GATCAGCGACCTGAAGAAGATCGAGGACCTGATCCAGAGCATGCACATCGACGCCACACTGTAC
ACCGAGTCCGATGTGCACCCTAGCTGCAAAGTGACCGCCATGAAGTGCTTTCTGCTGGAACTGC
AAGTGATCAGCCTGGAAAGCGGCGACGCCAGCATCCACGATACCGTGGAAAACCTGATCATCCT
GGCCAACAACAGCCTGAGCAGCAACGGCAATGTGACCGAGAGCGGCTGCAAAGAGTGCGAGGAA
CTGGAAGAGAAGAACATCAAAGAGTTCCTCCAGAGCTTCGTCCACATCGTGCAGATGTTCATCA
ACACCAGCTCTGGCGGAGGAAGCGGAGGCGGAGGATCTGGTGGTGGTGGATCTGGCGGCGGTGG
TAGTGGCGGAGGTTCTCTGCAAATCACCTGTCCTCCACCTATGAGCGTGGAACACGCCGACATC
TGGGTCAAGAGCTACAGCCTGTACAGCAGAGAGCGGTACATCTGCAACAGCGGCTTCAAGAGAA
AGGCCGGCACAAGCAGCCTGACCGAGTGTGTGCTGAACAAGGCCACAAACGTGGCCCACTGGAC
CACACCTAGCCTGAAGTGCATCAGAGATCCCGCTCTGGTTCATCAGAGGCCTGCCCCTCCATCT
ACAGTGACAACAGCTGGCGTGACCCCTCAGCCTGAGTCTCTGTCTCCATCTGGAAAAGAGCCTG
CCGCCAGCTCTCCCAGCTCTAACAATACTGCTGCCACCACAGCCGCTATCGTGCCTGGATCTCA
GCTGATGCCTAGCAAGAGCCCTAGCACCGGCACAACAGAGATCAGCTCTCACGAGAGCAGCCAC
GGAACACCTTCTCAGACCACCGCCAAGAATTGGGAGCTGACAGCCTCTGCCTCTCATCAGCCAC
CTGGCGTGTACCCACAGGGCCACTCTGATACAACAGTGGCCATCAGCACCAGCACCGTTCTGCT
GTGTGGCCTGTCTGCTGTTAGCCTGCTGGCCTGCTACCTGAAGTCTAGACAGACACCTCCTCTG
GCCAGCGTGGAAATGGAAGCCATGGAAGCTCTGCCTGTCACATGGGGCACCAGCAGCAGAGATG
AGGACCTCGAGAATTGCAGCCACCACCTG
exemplary CD19 CAR cargo sequence
SEQ ID NO: 175
ATGCTTCTCCTGGTGACAAGCCTTCTGCTCTGTGAGTTACCACACCCAGCATTCCTCCTGATCC
CAGACATCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCAGATGGA
ACTGTTAAACTCCTGATCTACCATACATCAAGATTACACTCAGGAGTCCCATCAAGGTTCAGTG
GCAGTGGGTCTGGAACAGATTATTCTCTCACCATTAGCAACCTGGAGCAAGAAGATATTGCCAC
TTACTTTTGCCAACAGGGTAATACGCTTCCGTACACGTTCGGAGGGGGGACTAAGTTGGAAATA
ACAGGCTCCACCTCTGGATCCGGCAAGCCCGGATCTGGCGAGGGATCCACCAAGGGCGAGGTGA
AACTGCAGGAGTCAGGACCTGGCCTGGTGGCGCCCTCACAGAGCCTGTCCGTCACATGCACTGT
CTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGAC
TGACCATCATCAAGGACAACTCCAAGAGCCAAGTTTTCTTAAAAATGAACAGTCTGCAAACTGA
TGACACAGCCATTTACTACTGTGCCAAACATTATTACTACGGTGGTAGCTATGCTATGGACTAC
TGGGGTCAAGGAACCTCAGTCACCGTCTCCTCAGCGGCCGCAATTGAAGTTATGTATCCTCCTC
CTTACCTAGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGGGGAGTCCTG
GCTTGCTATAGCTTGCTAGTAACAGTGGCCTTTATTATTTTCTGGGTGAGGAGTAAGAGGAGCA
GGCTCCTGCACAGTGACTACATGAACATGACTCCCCGCCGCCCCGGGCCCACCCGCAAGCATTA
CCAGCCCTATGCCCCACCACGCGACTTCGCAGCCTATCGCTCCAGAGTGAAGTTCAGCAGGAGC
GCAGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTCGCTAA
exemplary EGFR CAR cargo sequence
SEQ ID NO: 176
ATGGCACTCCCCGTCACCGCCCTTCTCTTGCCCCTCGCCCTGCTGCTGCATGCTGCCAGGCCCA
TGGACGAAGTGCAGCTCGTGGAGTCCGGTGGAGGACTCGTCCAACCGGGCGGATCCCTTCGCTT
GTCCTGCGCCGCATCAGGCTTCAGCTTCACCAACTATGGCGTCCACTGGGTCAGACAGGCCCCC
GGAAAGGGACTGGAATGGGTGTCCGTGATCTGGAGCGGCGGGAACACCGACTACAACACCTCCG
TGAAGGGCCGGTTCACTATTAGCCGCGACAACTCCAAGAACACTCTGTACCTCCAAATGAACTC
CCTGAGGGCCGAAGATACTGCTGTGTACTATTGCGCGAGAGCCCTGACCTACTACGACTACGAG
TTCGCGTACTGGGGCCAGGGGACTCTCGTGACCGTGTCCAGCGGTGGTGGAGGTTCCGGAGGCG
GAGGTTCTGGTGGCGGGGGATCAGAAATCGTGCTGACTCAGTCCCCTGCGACCTTGTCCCTGAG
CCCTGGAGAACGGGCCACCCTGAGCTGTAGAGCCAGCCAGAGCATCGGGACAAATATTCACTGG
TACCAGCAGAAACCCGGACAAGCACCACGGCTGCTGATCTACTACGCCTCCGAGTCGATTTCCG
GAATCCCGGCTCGCTTTTCGGGGTCTGGATCGGGAACGGACTTCACTCTGACCATCTCGTCGCT
GGAACCCGAGGATTTCGCCGTGTACTACTGCCAACAGAACAACAATTGGCCGACCACGTTCGGC
CAGGGCACCAAGCTCGAGATTAAGGGATCACTGGAAGCGGCCGCAACCACAACACCTGCTCCAA
GGCCCCCCACACCCGCTCCAACTATAGCCAGCCAACCATTGAGCCTCAGACCTGAAGCTTGCAG
GCCCGCAGCAGGAGGCGCCGTCCATACGCGAGGCCTGGACTTCGCGTGTGATATTTATATTTGG
GCCCCTTTGGCCGGAACATGTGGGGTGTTGCTTCTCTCCCTTGTGATCACTCTGTATTGTAAGC
GCGGGAGAAAGAAGCTCCTGTACATCTTCAAGCAGCCTTTTATGCGACCTGTGCAAACCACTCA
GGAAGAAGATGGGTGTTCATGCCGCTTCCCCGAGGAGGAAGAAGGAGGGTGTGAACTGAGGGTG
AAATTTTCTAGAAGCGCCGATGCTCCCGCATATCAGCAGGGTCAGAATCAGCTCTACAATGAAT
TGAATCTCGGCAGGCGAGAAGAGTACGATGTTCTGGACAAGAGACGGGGCAGGGATCCCGAGAT
GGGGGGAAAGCCCCGGAGAAAAAATCCTCAGGAGGGGTTGTACAATGAGCTGCAGAAGGACAAG
ATGGCTGAAGCCTATAGCGAGATCGGAATGAAAGGCGAAAGACGCAGAGGCAAGGGGCATGACG
GTCTGTACCAGGGTCTCTCTACAGCCACCAAGGACACTTATGATGCGTTGCATATGCAAGCCTT
GCCACCCCGCTAA
exemplary GFP cargo sequence
SEQ ID NO: 177
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAGTGA
exemplary CXCR1 cargo sequence
SEQ ID NO: 178
ATGTCAAATATTACAGATCCACAGATGTGGGATTTTGATGATCTAAATTTCACTGGCATGCCAC
CTGCAGATGAAGATTACAGCCCCTGTATGCTAGAAACTGAGACACTCAACAAGTATGTTGTGAT
CATCGCCTATGCCCTAGTGTTCCTGCTGAGCCTGCTGGGAAACTCCCTGGTGATGCTGGTCATC
TTATACAGCAGGGTCGGCCGCTCCGTCACTGATGTCTACCTGCTGAACCTGGCCTTGGCCGACC
TACTCTTTGCCCTGACCTTGCCCATCTGGGCCGCCTCCAAGGTGAATGGCTGGATTTTTGGCAC
ATTCCTGTGCAAGGTGGTCTCACTCCTGAAGGAAGTCAACTTCTACAGTGGCATCCTGCTGTTG
GCCTGCATCAGTGTGGACCGTTACCTGGCCATTGTCCATGCCACACGCACACTGACCCAGAAGC
GTCACTTGGTCAAGTTTGTTTGTCTTGGCTGCTGGGGACTGTCTATGAATCTGTCCCTGCCCTT
CTTCCTTTTCCGCCAGGCTTACCATCCAAACAATTCCAGTCCAGTTTGCTATGAGGTCCTGGGA
AATGACACAGCAAAATGGCGGATGGTGTTGCGGATCCTGCCTCACACCTTTGGCTTCATCGTGC
CGCTGTTTGTCATGCTGTTCTGCTATGGATTCACCCTGCGTACACTGTTTAAGGCCCACATGGG
GCAGAAGCACCGAGCCATGAGGGTCATCTTTGCTGTCGTCCTCATCTTCCTGCTTTGCTGGCTG
CCCTACAACCTGGTCCTGCTGGCAGACACCCTCATGAGGACCCAGGTGATCCAGGAGAGCTGTG
AGCGCCGCAACAACATCGGCCGGGCCCTGGATGCCACTGAGATTCTGGGATTTCTCCATAGCTG
CCTCAACCCCATCATCTACGCCTTCATCGGCCAAAATTTTCGCCATGGATTCCTCAAGATCCTG
GCTATGCATGGCCTGGTCAGCAAGGAGTTCTTGGCACGTCATCGTGTTACCTCCTACACTTCTT
CGTCTGTCAATGTCTCTTCCAACCTCTGA
exemplary CXCR3B cargo sequence
SEQ ID NO: 179
ATGGAGTTGAGGAAGTACGGCCCTGGAAGACTGGCGGGGACAGTTATAGGAGGAGCTGCTCAGA
GTAAATCACAGACTAAATCAGACTCAATCACAAAAGAGTTCCTGCCAGGCCTTTACACAGCCCC
TTCCTCCCCGTTCCCGCCCTCACAGGTGAGTGACCACCAAGTGCTAAATGACGCCGAGGTTGCC
GCCCTCCTGGAGAACTTCAGCTCTTCCTATGACTATGGAGAAAACGAGAGTGACTCGTGCTGTA
CCTCCCCGCCCTGCCCACAGGACTTCAGCCTGAACTTCGACCGGGCCTTCCTGCCAGCCCTCTA
CAGCCTCCTCTTTCTGCTGGGGCTGCTGGGCAACGGCGCGGTGGCAGCCGTGCTGCTGAGCCGG
CGGACAGCCCTGAGCAGCACCGACACCTTCCTGCTCCACCTAGCTGTAGCAGACACGCTGCTGG
TGCTGACACTGCCGCTCTGGGCAGTGGACGCTGCCGTCCAGTGGGTCTTTGGCTCTGGCCTCTG
CAAAGTGGCAGGTGCCCTCTTCAACATCAACTTCTACGCAGGAGCCCTCCTGCTGGCCTGCATC
AGCTTTGACCGCTACCTGAACATAGTTCATGCCACCCAGCTCTACCGCCGGGGGCCCCCGGCCC
GCGTGACCCTCACCTGCCTGGCTGTCTGGGGGCTCTGCCTGCTTTTCGCCCTCCCAGACTTCAT
CTTCCTGTCGGCCCACCACGACGAGCGCCTCAACGCCACCCACTGCCAATACAACTTCCCACAG
GTGGGCCGCACGGCTCTGCGGGTGCTGCAGCTGGTGGCTGGCTTTCTGCTGCCCCTGCTGGTCA
TGGCCTACTGCTATGCCCACATCCTGGCCGTGCTGCTGGTTTCCAGGGGCCAGCGGCGCCTGCG
GGCCATGCGGCTGGTGGTGGTGGTCGTGGTGGCCTTTGCCCTCTGCTGGACCCCCTATCACCTG
GTGGTGCTGGTGGACATCCTCATGGACCTGGGCGCTTTGGCCCGCAACTGTGGCCGAGAAAGCA
GGGTAGACGTGGCCAAGTCGGTCACCTCAGGCCTGGGCTACATGCACTGCTGCCTCAACCCGCT
GCTCTATGCCTTTGTAGGGGTCAAGTTCCGGGAGCGGATGTGGATGCTGCTCTTGCGCCTGGGC
TGCCCCAACCAGAGAGGGCTCCAGAGGCAGCCATCGTCTTCCCGCCGGGATTCATCCTGGTCTG
AGACCTCAGAGGCCTCCTACTCGGGCTTGTGA
exemplary CXCR3A cargo sequence
SEQ ID NO: 180
ATGGTCCTTGAGGTGAGTGACCACCAAGTGCTAAATGACGCCGAGGTTGCCGCCCTCCTGGAGA
ACTTCAGCTCTTCCTATGACTATGGAGAAAACGAGAGTGACTCGTGCTGTACCTCCCCGCCCTG
CCCACAGGACTTCAGCCTGAACTTCGACCGGGCCTTCCTGCCAGCCCTCTACAGCCTCCTCTTT
CTGCTGGGGCTGCTGGGCAACGGCGCGGTGGCAGCCGTGCTGCTGAGCCGGCGGACAGCCCTGA
GCAGCACCGACACCTTCCTGCTCCACCTAGCTGTAGCAGACACGCTGCTGGTGCTGACACTGCC
GCTCTGGGCAGTGGACGCTGCCGTCCAGTGGGTCTTTGGCTCTGGCCTCTGCAAAGTGGCAGGT
GCCCTCTTCAACATCAACTTCTACGCAGGAGCCCTCCTGCTGGCCTGCATCAGCTTTGACCGCT
ACCTGAACATAGTTCATGCCACCCAGCTCTACCGCCGGGGGCCCCCGGCCCGCGTGACCCTCAC
CTGCCTGGCTGTCTGGGGGCTCTGCCTGCTTTTCGCCCTCCCAGACTTCATCTTCCTGTCGGCC
CACCACGACGAGCGCCTCAACGCCACCCACTGCCAATACAACTTCCCACAGGTGGGCCGCACGG
CTCTGCGGGTGCTGCAGCTGGTGGCTGGCTTTCTGCTGCCCCTGCTGGTCATGGCCTACTGCTA
TGCCCACATCCTGGCCGTGCTGCTGGTTTCCAGGGGCCAGCGGCGCCTGCGGGCCATGCGGCTG
GTGGTGGTGGTCGTGGTGGCCTTTGCCCTCTGCTGGACCCCCTATCACCTGGTGGTGCTGGTGG
ACATCCTCATGGACCTGGGCGCTTTGGCCCGCAACTGTGGCCGAGAAAGCAGGGTAGACGTGGC
CAAGTCGGTCACCTCAGGCCTGGGCTACATGCACTGCTGCCTCAACCCGCTGCTCTATGCCTTT
GTAGGGGTCAAGTTCCGGGAGCGGATGTGGATGCTGCTCTTGCGCCTGGGCTGCCCCAACCAGA
GAGGGCTCCAGAGGCAGCCATCGTCTTCCCGCCGGGATTCATCCTGGTCTGAGACCTCAGAGGC
CTCCTACTCGGGCTTGTGA
exemplary CCR5 cargo sequence
SEQ ID NO: 181
ATGGATTATCAAGTGTCAAGTCCAATCTATGACATCAATTATTATACATCGGAGCCCTGCCAAA
AAATCAATGTGAAGCAAATCGCAGCCCGCCTCCTGCCTCCGCTCTACTCACTGGTGTTCATCTT
TGGTTTTGTGGGCAACATGCTGGTCATCCTCATCCTGATAAACTGCAAAAGGCTGAAGAGCATG
ACTGACATCTACCTGCTCAACCTGGCCATCTCTGACCTGTTTTTCCTTCTTACTGTCCCCTTCT
GGGCTCACTATGCTGCCGCCCAGTGGGACTTTGGAAATACAATGTGTCAACTCTTGACAGGGCT
CTATTTTATAGGCTTCTTCTCTGGAATCTTCTTCATCATCCTCCTGACAATCGATAGGTACCTG
GCTGTCGTCCATGCTGTGTTTGCTTTAAAAGCCAGGACGGTCACCTTTGGGGTGGTGACAAGTG
TGATCACTTGGGTGGTGGCTGTGTTTGCGTCTCTCCCAGGAATCATCTTTACCAGATCTCAAAA
AGAAGGTCTTCATTACACCTGCAGCTCTCATTTTCCATACAGTCAGTATCAATTCTGGAAGAAT
TTCCAGACATTAAAGATAGTCATCTTGGGGCTGGTCCTGCCGCTGCTTGTCATGGTCATCTGCT
ACTCGGGAATCCTAAAAACTCTGCTTCGGTGTCGAAATGAGAAGAAGAGGCACAGGGCTGTGAG
GCTTATCTTCACCATCATGATTGTTTATTTTCTCTTCTGGGCTCCCTACAACATTGTCCTTCTC
CTGAACACCTTCCAGGAATTCTTTGGCCTGAATAATTGCAGTAGCTCTAACAGGTTGGACCAAG
CTATGCAGGTGACAGAGACTCTTGGGATGACGCACTGCTGCATCAACCCCATCATCTATGCCTT
TGTCGGGGAGAAGTTCAGAAACTACCTCTTAGTCTTCTTCCAAAAGCACATTGCCAAACGCTTC
TGCAAATGCTGTTCTATTTTCCAGCAAGAGGCTCCCGAGCGAGCAAGCTCAGTTTACACCCGAT
CCACTGGGGAGCAGGAAATATCTGTGGGCTTGTGA
exemplary CCR2 cargo sequence
SEQ ID NO: 182
ATGCTGTCCACATCTCGTTCTCGGTTTATCAGAAATACCAACGAGAGCGGTGAAGAAGTCACCA
CCTTTTTTGATTATGATTACGGTGCTCCCTGTCATAAATTTGACGTGAAGCAAATTGGGGCCCA
ACTCCTGCCTCCGCTCTACTCGCTGGTGTTCATCTTTGGTTTTGTGGGCAACATGCTGGTCGTC
CTCATCTTAATAAACTGCAAAAAGCTGAAGTGCTTGACTGACATTTACCTGCTCAACCTGGCCA
TCTCTGATCTGCTTTTTCTTATTACTCTCCCATTGTGGGCTCACTCTGCTGCAAATGAGTGGGT
CTTTGGGAATGCAATGTGCAAATTATTCACAGGGCTGTATCACATCGGTTATTTTGGCGGAATC
TTCTTCATCATCCTCCTGACAATCGATAGATACCTGGCTATTGTCCATGCTGTGTTTGCTTTAA
AAGCCAGGACGGTCACCTTTGGGGTGGTGACAAGTGTGATCACCTGGTTGGTGGCTGTGTTTGC
TTCTGTCCCAGGAATCATCTTTACTAAATGCCAGAAAGAAGATTCTGTTTATGTCTGTGGCCCT
TATTTTCCACGAGGATGGAATAATTTCCACACAATAATGAGGAACATTTTGGGGCTGGTCCTGC
CGCTGCTCATCATGGTCATCTGCTACTCGGGAATCCTGAAAACCCTGCTTCGGTGTCGAAACGA
GAAGAAGAGGCATAGGGCAGTGAGAGTCATCTTCACCATCATGATTGTTTACTTTCTCTTCTGG
ACTCCCTATAATATTGTCATTCTCCTGAACACCTTCCAGGAATTCTTCGGCCTGAGTAACTGTG
AAAGCACCAGTCAACTGGACCAAGCCACGCAGGTGACAGAGACTCTTGGGATGACTCACTGCTG
CATCAATCCCATCATCTATGCCTTCGTTGGGGAGAAGTTCAGAAGCCTTTTTCACATAGCTCTT
GGCTGTAGGATTGCCCCACTCCAAAAACCAGTGTGTGGAGGTCCAGGAGTGAGACCAGGAAAGA
ATGTGAAAGTGACTACACAAGGACTCCTCGATGGTCGTGGAAAAGGAAAGTCAATTGGCAGAGC
CCCTGAAGCCAGTCTTCAGGACAAAGAAGGAGCCTAG

In some embodiments, a gene product of interest comprises or consists of an amino acid sequence of any one of SEQ ID NOs: 161, 164, or 183-200. In some embodiments, a gene product of interest comprises or consists of an amino acid sequence that is at least 85%, 90%, 95%, 96%, 97%, 98%, 99%, 99.1%, 99.2%, 99.3%, 99.4%, 99.5%, 99.6%, 99.7%, 99.8%, or 99.9% identical to any one of SEQ ID NOs: 161, 164, or 183-200. In some embodiments, a gene product of interest comprises or consists of a functional variant of any one of SEQ ID NOs: 161, 164, or 183-200. In some embodiments, a gene product of interest comprises or consists of an amino acid sequence having 1, 2, 3, 4, 5, 6, 7, 8, 9, or 10 mutations (e.g., substitutions, insertions, and/or deletions) relative to any one of SEQ ID NOs: 161, 164, or 183-200.

exemplary linker amino acid sequence
SEQ ID NO: 183
SGGGSGGGGSGGGGSGGGGSGGGSLQ
exemplary CD16 amino acid sequence
SEQ ID NO: 184
MWQLLLPTALLLLVSAGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDWKDHKFKWRKDPQDK
exemplary CD47 amino acid sequence
SEQ ID NO: 185
MWPLVAALLLGSACCGSAQLLENKTKSVEFTFCNDTVVIPCFVTNMEAQNTTEVYVKWKFKGRD
IYTFDGALNKSTVPTDFSSAKIEVSQLLKGDASLKMDKSDAVSHTGNYTCEVTELTREGETIIE
LKYRVVSWFSPNENILIVIFPIFAILLFWGQFGIKTLKYRSGGMDEKTIALLVAGLVITVIVIV
GAILFVPGEYSLKNATGLGLIVTSTGILILLHYYVFSTAIGLTSFVIAILVIQVIAYILAVVGL
SLCIAACIPMHGPLLISGLSILALAQLLGLVYMKFVASNQKTIQPPRKAVEEPLNAFKESKGMM
NDE
exemplary IL15 amino acid sequence
SEQ ID NO: 186
NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS
exemplary IgE-IL15 amino acid sequence
SEQ ID NO: 187
MDWTWILFLVAAATRVHSNWVNVISDLKKIEDLIQSMHIDATLYTESDVHPSCKVTAMKCELLE
LQVISLESGDASIHDTVENLIILANNSLSSNGNVTESGCKECEELEEKNIKEFLQSFVHIVQMF
INTS
exemplary IgE-IL15 pro-peptide amino acid sequence
SEQ ID NO: 188
MDWTWILFLVAAATRVHSGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCKECEEL
EEKNIKEFLQSFVHIVQMFINTS
exemplary IL15Rα amino acid sequence
SEQ ID NO: 189
ITCPPPMSVEHADIWVKSYSLYSRERYICNSGFKRKAGTSSLTECVLNKATNVAHWTTPSLKCI
RDPALVHQRPAPPSTVTTAGVTPQPESLSPSGKEPAASSPSSNNTAATTAAIVPGSQLMPSKSP
STGTTEISSHESSHGTPSQTTAKNWELTASASHQPPGVYPQGHSDTTVAISTSTVLLCGLSAVS
LLACYLKSRQTPPLASVEMEAMEALPVTWGTSSRDEDLENCSHHL
exemplary mbIL-15 amino acid sequence
SEQ ID NO: 190
MDWTWILFLVAAATRVHSNWVNVISDLKKIEDLIQSMHIDATLYTESDVHPSCKVTAMKCELLE
LQVISLESGDASIHDTVENLIILANNSLSSNGNVTESGCKECEELEEKNIKEFLQSFVHIVQME
INTSSGGGSGGGGSGGGGSGGGGSGGGSLQITCPPPMSVEHADIWVKSYSLYSRERYICNSGFK
RKAGTSSLTECVLNKATNVAHWTTPSLKCIRDPALVHQRPAPPSTVTTAGVTPQPESLSPSGKE
PAASSPSSNNTAATTAAIVPGSQLMPSKSPSTGTTEISSHESSHGTPSQTTAKNWELTASASHQ
PPGVYPQGHSDTTVAISTSTVLLCGLSAVSLLACYLKSRQTPPLASVEMEAMEALPVTWGTSSR
DEDLENCSHHL
exemplary mbIL-15 amino acid sequence
SEQ ID NO: 191
MDWTWILFLVAAATRVHSGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCKECEEL
EEKNIKEFLQSFVHIVQMFINTSSGGGSGGGGSGGGGSGGGGSGGGSLQITCPPPMSVEHADIW
VKSYSLYSRERYICNSGFKRKAGTSSLTECVLNKATNVAHWTTPSLKCIRDPALVHQRPAPPST
VTTAGVTPQPESLSPSGKEPAASSPSSNNTAATTAAIVPGSQLMPSKSPSTGTTEISSHESSHG
TPSQTTAKNWELTASASHQPPGVYPQGHSDTTVAISTSTVLLCGLSAVSLLACYLKSRQTPPLA
SVEMEAMEALPVTWGTSSRDEDLENCSHHL
exemplary multicistronic CD16, mbIL-15 amino acid sequence
SEQ ID NO: 192
MWQLLLPTALLLLVSAGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDWKDHKFKWRKDPQDKGS
GATNFSLLKQAGDVEENPGPMDWTWILFLVAAATRVHSNWVNVISDLKKIEDLIQSMHIDATLY
TESDVHPSCKVTAMKCFLLELQVISLESGDASIHDTVENLIILANNSLSSNGNVTESGCKECEE
LEEKNIKEFLQSFVHIVQMFINTSSGGGSGGGGSGGGGSGGGGSGGGSLQITCPPPMSVEHADI
WVKSYSLYSRERYICNSGFKRKAGTSSLTECVLNKATNVAHWTTPSLKCIRDPALVHQRPAPPS
TVTTAGVTPQPESLSPSGKEPAASSPSSNNTAATTAAIVPGSQLMPSKSPSTGTTEISSHESSH
GTPSQTTAKNWELTASASHQPPGVYPQGHSDTTVAISTSTVLLCGLSAVSLLACYLKSRQTPPL
ASVEMEAMEALPVTWGTSSRDEDLENCSHHL
exemplary CD19 CAR amino acid sequence
SEQ ID NO: 193
MLLLVTSLLLCELPHPAFLLIPDIQMTQTTSSLSASLGDRVTISCRASQDISKYLNWYQQKPDG
TVKLLIYHTSRLHSGVPSRFSGSGSGTDYSLTISNLEQEDIATYFCQQGNTLPYTFGGGTKLEI
TGSTSGSGKPGSGEGSTKGEVKLQESGPGLVAPSQSLSVTCTVSGVSLPDYGVSWIRQPPRKGL
EWLGVIWGSETTYYNSALKSRLTIIKDNSKSQVFLKMNSLQTDDTAIYYCAKHYYYGGSYAMDY
WGQGTSVTVSSAAAIEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRDFAAYRSRVKESRS
ADAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
exemplary EGFR CAR amino acid sequence
SEQ ID NO: 194
MALPVTALLLPLALLLHAARPMDEVQLVESGGGLVQPGGSLRLSCAASGFSFTNYGVHWVRQAP
GKGLEWVSVIWSGGNTDYNTSVKGRFTISRDNSKNTLYLQMNSLRAEDTAVYYCARALTYYDYE
FAYWGQGTLVTVSSGGGGSGGGGSGGGGSEIVLTQSPATLSLSPGERATLSCRASQSIGTNIHW
YQQKPGQAPRLLIYYASESISGIPARFSGSGSGTDFTLTISSLEPEDFAVYYCQQNNNWPTTFG
QGTKLEIKGSLEAAATTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
exemplary GFP amino acid sequence
SEQ ID NO: 195
MVSKGEELFTGVVPILVELDGDVNGHKESVSGEGEGDATYGKLTLKFICTTGKLPVPWPTLVTT
LTYGVQCFSRYPDHMKQHDFFKSAMPEGYVQERTIFFKDDGNYKTRAEVKFEGDTLVNRIELKG
IDFKEDGNILGHKLEYNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK
exemplary CXCR1 amino acid sequence
SEQ ID NO: 196
MSNITDPQMWDFDDLNFTGMPPADEDYSPCMLETETLNKYVVIIAYALVFLLSLLGNSLVMLVI
LYSRVGRSVTDVYLLNLALADLLFALTLPIWAASKVNGWIFGTFLCKVVSLLKEVNFYSGILLL
ACISVDRYLAIVHATRTLTQKRHLVKFVCLGCWGLSMNLSLPFFLFRQAYHPNNSSPVCYEVLG
NDTAKWRMVLRILPHTFGFIVPLFVMLFCYGFTLRTLFKAHMGQKHRAMRVIFAVVLIFLLCWL
PYNLVLLADTLMRTQVIQESCERRNNIGRALDATEILGFLHSCLNPIIYAFIGQNFRHGELKIL
AMHGLVSKEFLARHRVTSYTSSSVNVSSNL
exemplary CXCR3B amino acid sequence
SEQ ID NO: 197
MELRKYGPGRLAGTVIGGAAQSKSQTKSDSITKEFLPGLYTAPSSPFPPSQVSDHQVLNDAEVA
ALLENFSSSYDYGENESDSCCTSPPCPQDESLNFDRAFLPALYSLLELLGLLGNGAVAAVLLSR
RTALSSTDTELLHLAVADTLLVLTLPLWAVDAAVQWVFGSGLCKVAGALFNINFYAGALLLACI
SFDRYLNIVHATQLYRRGPPARVILTCLAVWGLCLLFALPDFIFLSAHHDERLNATHCQYNFPQ
VGRTALRVLQLVAGFLLPLLVMAYCYAHILAVLLVSRGQRRLRAMRLVVVVVVAFALCWTPYHL
VVLVDILMDLGALARNCGRESRVDVAKSVTSGLGYMHCCLNPLLYAFVGVKFRERMWMLLLRLG
CPNQRGLQRQPSSSRRDSSWSETSEASYSGL
exemplary CXCR3A amino acid sequence
SEQ ID NO: 198
MVLEVSDHQVLNDAEVAALLENFSSSYDYGENESDSCCTSPPCPQDESLNFDRAFLPALYSLLF
LLGLLGNGAVAAVLLSRRTALSSTDTFLLHLAVADTLLVLTLPLWAVDAAVQWVFGSGLCKVAG
ALFNINFYAGALLLACISFDRYLNIVHATQLYRRGPPARVTLTCLAVWGLCLLFALPDFIFLSA
HHDERLNATHCQYNFPQVGRTALRVLQLVAGFLLPLLVMAYCYAHILAVLLVSRGQRRLRAMRL
VVVVVVAFALCWTPYHLVVLVDILMDLGALARNCGRESRVDVAKSVTSGLGYMHCCLNPLLYAF
VGVKFRERMWMLLLRLGCPNQRGLQRQPSSSRRDSSWSETSEASYSGL
exemplary CCR5 amino acid sequence
SEQ ID NO: 199
MDYQVSSPIYDINYYTSEPCQKINVKQIAARLLPPLYSLVFIFGFVGNMLVILILINCKRLKSM
TDIYLLNLAISDLFFLLTVPFWAHYAAAQWDFGNTMCQLLTGLYFIGFFSGIFFIILLTIDRYL
AVVHAVFALKARTVTFGVVTSVITWVVAVFASLPGIIFTRSQKEGLHYTCSSHFPYSQYQFWKN
FQTLKIVILGLVLPLLVMVICYSGILKTLLRCRNEKKRHRAVRLIFTIMIVYFLFWAPYNIVLL
LNTFQEFFGLNNCSSSNRLDQAMQVTETLGMTHCCINPIIYAFVGEKFRNYLLVFFQKHIAKRE
CKCCSIFQQEAPERASSVYTRSTGEQEISVGL
exemplary CCR2 cargo sequence
SEQ ID NO: 200
MLSTSRSRFIRNTNESGEEVTTFFDYDYGAPCHKEDVKQIGAQLLPPLYSLVFIFGFVGNMLVV
LILINCKKLKCLTDIYLLNLAISDLLFLITLPLWAHSAANEWVEGNAMCKLFTGLYHIGYFGGI
FFIILLTIDRYLAIVHAVFALKARTVTFGVVTSVITWLVAVFASVPGIIFTKCQKEDSVYVCGP
YFPRGWNNFHTIMRNILGLVLPLLIMVICYSGILKTLLRCRNEKKRHRAVRVIFTIMIVYFLEW
TPYNIVILLNTFQEFFGLSNCESTSQLDQATQVTETLGMTHCCINPIIYAFVGEKERSLFHIAL
GCRIAPLQKPVCGGPGVRPGKNVKVTTQGLLDGRGKGKSIGRAPEASLQDKEGA

### AAV Capsids

In some embodiments, the present disclosure provides one or more polynucleotide constructs (e.g., knock-in cassettes) packaged into an AAV capsid. In some embodiments, an AAV capsid is from or derived from an AAV capsid of an AAV2, 3, 4, 5, 6, 7, 8, 9, or 10 serotype, or one or more hybrids thereof. In some embodiments, an AAV capsid is from an AAV ancestral serotype. In some embodiments, an AAV capsid is an ancestral (Anc) AAV capsid. An Anc capsid is created from a construct sequence that is constructed using evolutionary probabilities and evolutionary modeling to determine a probable ancestral sequence. In some embodiments, an AAV capsid has been modified in a manner known in the art (see e.g., Büning and Srivastava, Capsid modifications for targeting and improving the efficacy of AAV vectors, Mol Ther Methods Clin Dev. 2019)

In some embodiments, as provided herein, any combination of AAV capsids and AAV constructs (e.g., comprising AAV ITRs) may be used in recombinant AAV (rAAV) particles of the present disclosure. In some embodiments, an AAV ITR is from or derived from an AAV ITR of AAV2, 3, 4, 5, 6, 7, 8, 9, or 10. For example, wild-type or variant AA6 ITRs and AAV6 capsid, wild-type or variant AAV2 ITRs and AAV6 capsid, etc. In some embodiments of the present disclosure, an AAV particle is wholly comprised of AAV6 components (e.g., capsid and ITRs are AAV6 serotype). In some embodiments, an AAV particle is an AAV6/2, AAV6/8 or AAV6/9 particle (e.g., an AAV2, AAV8 or AAV9 capsid with an AAV construct having AAV6 ITRs).

### Exemplary AAV Constructs

In some embodiments, a donor template is included within an AAV construct. In some embodiments, an AAV construct sequence comprises or consists of the sequence of any one of SEQ ID NO: 201-204. In some embodiments, an exemplary AAV construct is represented by SEQ ID NO:201. In some embodiments, an exemplary AAV construct is represented by SEQ ID NO: 202. In some embodiments, an exemplary AAV construct is represented by SEQ ID NO: 203. In some embodiments, an exemplary AAV construct is represented by SEQ ID NO: 204. In some embodiments, an exemplary AAV construct is at least 80%, 85%, 90%, 95%, 98%, or 99% identical to a sequence represented by SEQ ID NO: 201-204.

exemplary AAV construct for donor template insertion at GAPDH locus
SEQ ID NO: 201
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGTGGCAACTGCTGCTGCCTACAGCTCTGCTGCTTCTGGTGTCTGCCGGCATGAGAACCGAGG
ATCTGCCTAAGGCCGTGGTGTTCCTGGAACCTCAGTGGTACAGAGTGCTGGAAAAGGACAGCGT
GACCCTGAAGTGCCAGGGCGCCTATTCTCCCGAGGACAATAGCACCCAGTGGTTCCACAACGAG
AGCCTGATCAGCAGCCAGGCCAGCAGCTACTTTATCGATGCCGCCACCGTGGACGACAGCGGCG
AGTACAGATGCCAGACCAATCTGAGCACCCTGAGCGACCCTGTGCAGCTGGAAGTGCACATTGG
ATGGTTGCTGCTGCAAGCCCCTAGATGGGTGTTCAAAGAAGAGGACCCCATCCACCTGAGATGC
CACTCTTGGAAGAACACAGCCCTGCACAAAGTGACCTACCTGCAGAACGGCAAGGGCAGAAAGT
ACTTCCACCACAACAGCGACTTCTACATCCCCAAGGCCACACTGAAGGACTCCGGCTCCTACTT
CTGCAGAGGCCTGGTCGGCAGCAAGAACGTGTCCAGCGAGACAGTGAACATCACCATCACACAG
GGCCTCGCCGTGTCTACCATCAGCAGCTTTTTCCCACCTGGCTATCAGGTGTCCTTCTGCCTGG
TCATGGTGCTGCTGTTCGCCGTGGATACCGGCCTGTACTTCAGCGTCAAGACCAACATCCGGTC
CAGCACCAGAGACTGGAAGGACCACAAGTTCAAGTGGCGGAAGGACCCTCAGGACAAGTAAGCG
GCCGCGTCGAGTCTAGAGGGCCCGTTTAAACCCGCTGATCAGCCTCGACTGTGCCTTCTAGTTG
CCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGA
TGCGGTGGGCTCTATGGATTTGGCTACAGCAACAGGGTGGTGGACCTCATGGCCCACATGGCCT
CCAAGGAGTAAGACCCCTGGACCACCAGCCCCAGCAAGAGCACAAGAGGAAGAGAGAGACCCTC
ACTGCTGGGGAGTCCCTGCCACACTCAGTCCCCCACCACACTGAATCTCCCCTCCTCACAGTTG
CCATGTAGACCCCTTGAAGAGGGGAGGGGCCTAGGGAGCCGCACCTTGTCATGTACCATCAATA
AAGTACCCTGTGCTCAACCAGTTACTTGTCCTGTCTTATTCTAGGGTCTGGGGCAGAGGGGAGG
GAAGCTGGGCTTGTGTCAAGGTGAGACATTCTTGCTGGGGAGGGACCTGGTATGTTCTCCTCAG
ACTGAGGGTAGGGCCTCCAAACAGCCTTGCTTGCTTCGAGAACCATTTGCTTCCCGCTCAGACG
TCTTGAGTGCTACAGGAAGCTGGCACCACTACTTCAGAGAACAAGGCCTTTTCCTCTCCTCGCT
CCAGTAGATCTAGGAACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGCGGCCTCAGTGAGCG
AGCGAGCGCGCAGCTGCCTGCAGG
exemplary AAV construct for donor template insertion at GAPDH locus
SEQ ID NO: 202
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCCGCCACAA
CATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACG
AGAAGCGCGATCACATGGTCCTGCTGGAGTTCGTGACCGCCGCCGGGATCACTCTCGGCATGGA
CGAGCTGTACAAGTGAGCGGCCGCGTCGAGTCTAGAGGGCCCGTTTAAACCCGCTGATCAGCCT
CGACTGTGCCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGACA
ATAGCAGGCATGCTGGGGATGCGGTGGGCTCTATGGATTTGGCTACAGCAACAGGGTGGTGGAC
CTCATGGCCCACATGGCCTCCAAGGAGTAAGACCCCTGGACCACCAGCCCCAGCAAGAGCACAA
GAGGAAGAGAGAGACCCTCACTGCTGGGGAGTCCCTGCCACACTCAGTCCCCCACCACACTGAA
TCTCCCCTCCTCACAGTTGCCATGTAGACCCCTTGAAGAGGGGAGGGGCCTAGGGAGCCGCACC
TTGTCATGTACCATCAATAAAGTACCCTGTGCTCAACCAGTTACTTGTCCTGTCTTATTCTAGG
GTCTGGGGCAGAGGGGAGGGAAGCTGGGCTTGTGTCAAGGTGAGACATTCTTGCTGGGGAGGGA
CCTGGTATGTTCTCCTCAGACTGAGGGTAGGGCCTCCAAACAGCCTTGCTTGCTTCGAGAACCA
TTTGCTTCCCGCTCAGACGTCTTGAGTGCTACAGGAAGCTGGCACCACTACTTCAGAGAACAAG
GCCTTTTCCTCTCCTCGCTCCAGTAGATCTAGGAACCCCTAGTGATGGAGTTGGCCACTCCCTC
TCTGCGCGCTCGCTCGCTCACTGAGGCCGGGCGACCAAAGGTCGCCCGACGCCCGGGCTTTGCC
CGGGCGGCCTCAGTGAGCGAGCGAGCGCGCAGCTGCCTGCAGG
exemplary AAV construct for donor template insertion at GAPDH locus
SEQ ID NO: 203
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGCTTCTCCTGGTGACAAGCCTTCTGCTCTGTGAGTTACCACACCCAGCATTCCTCCTGATCC
CAGACATCCAGATGACACAGACTACATCCTCCCTGTCTGCCTCTCTGGGAGACAGAGTCACCAT
CAGTTGCAGGGCAAGTCAGGACATTAGTAAATATTTAAATTGGTATCAGCAGAAACCAGATGGA
ACTGTTAAACTCCTGATCTACCATACATCAAGATTACACTCAGGAGTCCCATCAAGGTTCAGTG
GCAGTGGGTCTGGAACAGATTATTCTCTCACCATTAGCAACCTGGAGCAAGAAGATATTGCCAC
TTACTTTTGCCAACAGGGTAATACGCTTCCGTACACGTTCGGAGGGGGGACTAAGTTGGAAATA
ACAGGCTCCACCTCTGGATCCGGCAAGCCCGGATCTGGCGAGGGATCCACCAAGGGCGAGGTGA
AACTGCAGGAGTCAGGACCTGGCCTGGTGGCGCCCTCACAGAGCCTGTCCGTCACATGCACTGT
CTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGAC
TGACCATCATCAAGGACAACTCCAAGAGCCAAGTTTTCTTAAAAATGAACAGTCTGCAAACTGA
TGACACAGCCATTTACTACTGTGCCAAACATTATTACTACGGTGGTAGCTATGCTATGGACTAC
TGGGGTCAAGGAACCTCAGTCACCGTCTCCTCAGCGGCCGCAATTGAAGTTATGTATCCTCCTC
CTTACCTAGACAATGAGAAGAGCAATGGAACCATTATCCATGTGAAAGGGAAACACCTTTGTCC
AAGTCCCCTATTTCCCGGACCTTCTAAGCCCTTTTGGGTGCTGGTGGTGGTTGGGGGAGTCCTG
GCTTGCTATAGCTTGCTAGTAACAGTGGCCTTTATTATTTTCTGGGTGAGGAGTAAGAGGAGCA
GGCTCCTGCACAGTGACTACATGAACATGACTCCCCGCCGCCCCGGGCCCACCCGCAAGCATTA
CCAGCCCTATGCCCCACCACGCGACTTCGCAGCCTATCGCTCCAGAGTGAAGTTCAGCAGGAGC
GCAGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTCGCTAAAG
CGGCCGCGTCGAGTCTAGAGGGCCCGTTTAAACCCGCTGATCAGCCTCGACTGTGCCTTCTAGT
TGCCAGCCATCTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTCATTCTATTCT
GGGGGGTGGGGTGGGGCAGGACAGCAAGGGGGAGGATTGGGAAGACAATAGCAGGCATGCTGGG
GATGCGGTGGGCTCTATGGATTTGGCTACAGCAACAGGGTGGTGGACCTCATGGCCCACATGGC
CTCCAAGGAGTAAGACCCCTGGACCACCAGCCCCAGCAAGAGCACAAGAGGAAGAGAGAGACCC
TCACTGCTGGGGAGTCCCTGCCACACTCAGTCCCCCACCACACTGAATCTCCCCTCCTCACAGT
TGCCATGTAGACCCCTTGAAGAGGGGAGGGGCCTAGGGAGCCGCACCTTGTCATGTACCATCAA
TAAAGTACCCTGTGCTCAACCAGTTACTTGTCCTGTCTTATTCTAGGGTCTGGGGCAGAGGGGA
GGGAAGCTGGGCTTGTGTCAAGGTGAGACATTCTTGCTGGGGAGGGACCTGGTATGTTCTCCTC
AGACTGAGGGTAGGGCCTCCAAACAGCCTTGCTTGCTTCGAGAACCATTTGCTTCCCGCTCAGA
CGTCTTGAGTGCTACAGGAAGCTGGCACCACTACTTCAGAGAACAAGGCCTTTTCCTCTCCTCG
CTCCAGTAGATCTAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGGCCTCAGTGAG
CGAGCGAGCGCGCAGCTGCCTGCAGG
exemplary AAV construct for donor template insertion at GAPDH locus
SEQ ID NO: 204
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGTCGACGAAGACTGTGGATGGCCCCTCCGGGAAACTGTGGCGTGATGGCCG
CGGGGCTCTCCAGAACATCATCCCTGCCTCTACTGGCGCTGCCAAGGCTGTGGGCAAGGTCATC
CCTGAGCTGAACGGGAAGCTCACTGGCATGGCCTTCCGTGTCCCCACTGCCAACGTGTCAGTGG
TGGACCTGACCTGCCGTCTAGAAAAACCTGCCAAATATGATGACATCAAGAAGGTGGTGAAGCA
GGCGTCGGAGGGCCCCCTCAAGGGCATCCTGGGCTACACTGAGCACCAGGTGGTCTCCTCTGAC
TTCAACAGCGACACCCACTCCTCCACCTTTGACGCTGGGGCTGGCATTGCCCTCAACGACCACT
TTGTCAAGCTCATTTCCTGGTATGTGGCTGGGGCCAGAGACTGGCTCTTAAAAAGTGCAGGGTC
TGGCGCCCTCTGGTGGCTGGCTCAGAAAAAGGGCCCTGACAACTCTTTACATCTTCTAGGTATG
ACAACGAGTTCGGATATAGCAATAGAGTGGTCGATCTGATGGCTCATATGGCTAGCAAAGAGGG
AAGCGGAGCTACTAACTTCAGCCTGCTGAAGCAGGCTGGAGACGTGGAGGAGAACCCTGGACCT
ATGGCACTCCCCGTCACCGCCCTTCTCTTGCCCCTCGCCCTGCTGCTGCATGCTGCCAGGCCCA
TGGACGAAGTGCAGCTCGTGGAGTCCGGTGGAGGACTCGTCCAACCGGGCGGATCCCTTCGCTT
GTCCTGCGCCGCATCAGGCTTCAGCTTCACCAACTATGGCGTCCACTGGGTCAGACAGGCCCCC
GGAAAGGGACTGGAATGGGTGTCCGTGATCTGGAGCGGCGGGAACACCGACTACAACACCTCCG
TGAAGGGCCGGTTCACTATTAGCCGCGACAACTCCAAGAACACTCTGTACCTCCAAATGAACTC
CCTGAGGGCCGAAGATACTGCTGTGTACTATTGCGCGAGAGCCCTGACCTACTACGACTACGAG
TTCGCGTACTGGGGCCAGGGGACTCTCGTGACCGTGTCCAGCGGTGGTGGAGGTTCCGGAGGCG
GAGGTTCTGGTGGCGGGGGATCAGAAATCGTGCTGACTCAGTCCCCTGCGACCTTGTCCCTGAG
CCCTGGAGAACGGGCCACCCTGAGCTGTAGAGCCAGCCAGAGCATCGGGACAAATATTCACTGG
TACCAGCAGAAACCCGGACAAGCACCACGGCTGCTGATCTACTACGCCTCCGAGTCGATTTCCG
GAATCCCGGCTCGCTTTTCGGGGTCTGGATCGGGAACGGACTTCACTCTGACCATCTCGTCGCT
GGAACCCGAGGATTTCGCCGTGTACTACTGCCAACAGAACAACAATTGGCCGACCACGTTCGGC
CAGGGCACCAAGCTCGAGATTAAGGGATCACTGGAAGCGGCCGCAACCACAACACCTGCTCCAA
GGCCCCCCACACCCGCTCCAACTATAGCCAGCCAACCATTGAGCCTCAGACCTGAAGCTTGCAG
GCCCGCAGCAGGAGGCGCCGTCCATACGCGAGGCCTGGACTTCGCGTGTGATATTTATATTTGG
GCCCCTTTGGCCGGAACATGTGGGGTGTTGCTTCTCTCCCTTGTGATCACTCTGTATTGTAAGC
GCGGGAGAAAGAAGCTCCTGTACATCTTCAAGCAGCCTTTTATGCGACCTGTGCAAACCACTCA
GGAAGAAGATGGGTGTTCATGCCGCTTCCCCGAGGAGGAAGAAGGAGGGTGTGAACTGAGGGTG
AAATTTTCTAGAAGCGCCGATGCTCCCGCATATCAGCAGGGTCAGAATCAGCTCTACAATGAAT
TGAATCTCGGCAGGCGAGAAGAGTACGATGTTCTGGACAAGAGACGGGGCAGGGATCCCGAGAT
GGGGGGAAAGCCCCGGAGAAAAAATCCTCAGGAGGGGTTGTACAATGAGCTGCAGAAGGACAAG
ATGGCTGAAGCCTATAGCGAGATCGGAATGAAAGGCGAAAGACGCAGAGGCAAGGGGCATGACG
GTCTGTACCAGGGTCTCTCTACAGCCACCAAGGACACTTATGATGCGTTGCATATGCAAGCCTT
GCCACCCCGCTAAAGCGGCCGCGTCGAGTCTAGAGGGCCCGTTTAAACCCGCTGATCAGCCTCG
ACTGTGCCTTCTAGTTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAAT
AGCAGGCATGCTGGGGATGCGGTGGGCTCTATGGATTTGGCTACAGCAACAGGGTGGTGGACCT
CATGGCCCACATGGCCTCCAAGGAGTAAGACCCCTGGACCACCAGCCCCAGCAAGAGCACAAGA
GGAAGAGAGAGACCCTCACTGCTGGGGAGTCCCTGCCACACTCAGTCCCCCACCACACTGAATC
TCCCCTCCTCACAGTTGCCATGTAGACCCCTTGAAGAGGGGAGGGGCCTAGGGAGCCGCACCTT
GTCATGTACCATCAATAAAGTACCCTGTGCTCAACCAGTTACTTGTCCTGTCTTATTCTAGGGT
CTGGGGCAGAGGGGAGGGAAGCTGGGCTTGTGTCAAGGTGAGACATTCTTGCTGGGGAGGGACC
TGGTATGTTCTCCTCAGACTGAGGGTAGGGCCTCCAAACAGCCTTGCTTGCTTCGAGAACCATT
TGCTTCCCGCTCAGACGTCTTGAGTGCTACAGGAAGCTGGCACCACTACTTCAGAGAACAAGGC
CTTTTCCTCTCCTCGCTCCAGTAGATCTAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGG

### Exemplary Donor Template Sequences

In some embodiments, a donor template comprises in 5′ to 3′ order, a target sequence 5′ homology arm (which optionally comprises an optimized sequence that is not a wild type sequence), a second regulatory element that enables expression of a cargo sequence as a separate translational product (e.g., an IRES sequence and/or a 2A element), a cargo sequence (e.g., a gene product of interest), optionally a second regulatory element that enables expression of a cargo sequence as a separate translational product (e.g., an IRES sequence and/or a 2A element), optionally a second cargo sequence (e.g., a gene product of interest), optionally a 3′ UTR, a poly adenylation signal (e.g., a BGHpA signal), and a target sequence 3′ homology arm (which optionally comprises an optimized sequence that is not a wild type sequence).

In some embodiments, a donor template comprises or consists of the sequence of any one of SEQ ID NOs: 38-57 and 205-218. In some embodiments, a donor template comprises or consists of a sequence that is at least 85%, 90%, 95%, 98% or 99% identical to any one of SEQ ID NOs: 38-57 and 205-218.

exemplary donor template for insertion at GAPDH locus
SEQ ID NO: 38
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGGAGGGCAGAGGAAGTCTT
CTAACATGCGGTGACGTGGAGGAGAATCCTGGCCCGATGGTGAGCAAGGGCGAGGAGGATAACA
TGGCCATCATCAAGGAGTTCATGCGCTTCAAGGTGCACATGGAGGGCTCCGTGAACGGCCACGA
GTTCGAGATCGAGGGCGAGGGCGAGGGCCGCCCCTACGAGGGCACCCAGACCGCCAAGCTGAAG
GTGACCAAGGGTGGCCCCCTGCCCTTCGCCTGGGACATCCTGTCCCCTCAGTTCATGTACGGCT
CCAAGGCCTACGTGAAGCACCCCGCCGACATCCCCGACTACTTGAAGCTGTCCTTCCCCGAGGG
CTTCAAGTGGGAGCGCGTGATGAACTTCGAGGACGGCGGCGTGGTGACCGTGACCCAGGACTCC
TCCCTGCAGGACGGCGAGTTCATCTACAAGGTGAAGCTGCGCGGCACCAACTTCCCCTCCGACG
GCCCCGTAATGCAGAAGAAGACAATGGGCTGGGAGGCCTCCTCCGAGCGGATGTACCCCGAGGA
CGGCGCCCTGAAGGGCGAGATCAAGCAGAGGCTGAAGCTGAAGGACGGCGGCCACTACGACGCT
GAGGTCAAGACCACCTACAAGGCCAAGAAGCCCGTGCAGCTGCCCGGCGCCTACAACGTCAACA
TCAAGTTGGACATCACCTCCCACAACGAGGACTACACCATCGTGGAACAGTACGAACGCGCCGA
GGGCCGCCACTCCACCGGCGGCATGGACGAGCTGTACAAGTAAGCGGCCGCGTCGAGTCTAGAG
GGCCCGTTTAAACCCGCTGATCAGCCTCGACTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGATGCGGTGGGCTCTATGGA
TTTGGCTACAGCAACAGGGTGGTGGACCTCATGGCCCACATGGCCTCCAAGGAGTAAGACCCCT
GGACCACCAGCCCCAGCAAGAGCACAAGAGGAAGAGAGAGACCCTCACTGCTGGGGAGTCCCTG
CCACACTCAGTCCCCCACCACACTGAATCTCCCCTCCTCACAGTTGCCATGTAGACCCCTTGAA
GAGGGGGGGGCCTAGGGAGCCGCACCTTGTCATGTACCATCAATAAAGTACCCTGTGCTCAAC
CAGTTACTTGTCCTGTCTTATTCTAGGGTCTGGGGCAGAGGGGAGGGAAGCTGGGCTTGTGTCA
AGGTGAGACATTCTTGCTGGGGAGGGACCTGGTATGTTCTCCTCAGACTGAGGGTAGGGCCTCC
AAACAGCCTTGCTTGCTTCGAGAACCATTTGCTTCCCGCTCAGACGTCTTGAGTGCTACAGGAA
GCTGGCACCACTACTTCAGAGAACAAGGCCTTTTCCTCTCCTCGCTCCAGT
exemplary donor template for insertion at GAPDH locus
SEQ ID NO: 39
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGTAACCC
CTCTCCCTCCCCCCCCCCTAACGTTACTGGCCGAAGCCGCTTGGAATAAGGCCGGTGTGCGTTT
GTCTATATGTTATTTTCCACCATATTGCCGTCTTTTGGCAATGTGAGGGCCCGGAAACCTGGCC
CTGTCTTCTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTGTAGCGACC
CTTTGCAGGCAGCGGAACCCCCCACCTGGCGACAGGTGCCTCTGCGGCCAAAAGCCACGTGTAT
AAGATACACCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGGTACCCCAT
TGTATGGGATCTGATCTGGGGCCTCGGTGCACATGCTTTACATGTGTTTAGTCGAGGTTAAAAA
AACGTCTAGGCCCCCCGAACCACGGGGACGTGGTTTTCCTTTGAAAAACACGATGATAAATGGT
GAGCAAGGGCGAGGAGGATAACATGGCCATCATCAAGGAGTTCATGCGCTTCAAGGTGCACATG
GAGGGCTCCGTGAACGGCCACGAGTTCGAGATCGAGGGCGAGGGCGAGGGCCGCCCCTACGAGG
GCACCCAGACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCCCGACTAC
TTGAAGCTGTCCTTCCCCGAGGGCTTCAAGTGGGAGCGCGTGATGAACTTCGAGGACGGCGGCG
TGGTGACCGTGACCCAGGACTCCTCCCTGCAGGACGGCGAGTTCATCTACAAGGTGAAGCTGCG
CGGCACCAACTTCCCCTCCGACGGCCCCGTAATGCAGAAGAAGACAATGGGCTGGGAGGCCTCC
TCCGAGCGGATGTACCCCGAGGACGGCGCCCTGAAGGGCGAGATCAAGCAGAGGCTGAAGCTGA
AGGACGGCGGCCACTACGACGCTGAGGTCAAGACCACCTACAAGGCCAAGAAGCCCGTGCAGCT
GCCCGGCGCCTACAACGTCAACATCAAGTTGGACATCACCTCCCACAACGAGGACTACACCATC
GTGGAACAGTACGAACGCGCCGAGGGCCGCCACTCCACCGGCGGCATGGACGAGCTGTACAAGT
AAGCGGCCGCGTCGAGTCTAGAGGGCCCGTTTAAACCCGCTGATCAGCCTCGACTGTGCCTTCT
AGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCT
GGGGATGCGGTGGGCTCTATGGATTTGGCTACAGCAACAGGGTGGTGGACCTCATGGCCCACAT
GGCCTCCAAGGAGTAAGACCCCTGGACCACCAGCCCCAGCAAGAGCACAAGAGGAAGAGAGAGA
CCCTCACTGCTGGGGAGTCCCTGCCACACTCAGTCCCCCACCACACTGAATCTCCCCTCCTCAC
AGTTGCCATGTAGACCCCTTGAAGAGGGGAGGGGCCTAGGGAGCCGCACCTTGTCATGTACCAT
CAATAAAGTACCCTGTGCTCAACCAGTTACTTGTCCTGTCTTATTCTAGGGTCTGGGGCAGAGG
GGAGGGAAGCTGGGCTTGTGTCAAGGTGAGACATTCTTGCTGGGGAGGGACCTGGTATGTTCTC
CTCAGACTGAGGGTAGGGCCTCCAAACAGCCTTGCTTGCTTCGAGAACCATTTGCTTCCCGCTC
AGACGTCTTGAGTGCTACAGGAAGCTGGCACCACTACTTCAGAGAACAAGGCCTTTTCCTCTCC
TCGCTCCAGT
exemplary donor template for insertion at GAPDH locus
SEQ ID NO: 40
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGGGAAGC
GGAGCTACTAACTTCAGCCTGCTGAAGCAGGCTGGAGACGTGGAGGAGAACCCTGGACCTATGG
TGAGCAAGGGCGAGGAGGATAACATGGCCATCATCAAGGAGTTCATGCGCTTCAAGGTGCACAT
GGAGGGCTCCGTGAACGGCCACGAGTTCGAGATCGAGGGCGAGGGCGAGGGCCGCCCCTACGAG
GGCACCCAGACCGCCAAGCTGAAGGTGACCAAGGGTGGCCCCCTGCCCTTCGCCTGGGACATCC
TGTCCCCTCAGTTCATGTACGGCTCCAAGGCCTACGTGAAGCACCCCGCCGACATCCCCGACTA
CTTGAAGCTGTCCTTCCCCGAGGGCTTCAAGTGGGAGCGCGTGATGAACTTCGAGGACGGCGGC
GTGGTGACCGTGACCCAGGACTCCTCCCTGCAGGACGGCGAGTTCATCTACAAGGTGAAGCTGC
GCGGCACCAACTTCCCCTCCGACGGCCCCGTAATGCAGAAGAAGACAATGGGCTGGGAGGCCTC
CTCCGAGCGGATGTACCCCGAGGACGGCGCCCTGAAGGGCGAGATCAAGCAGAGGCTGAAGCTG
AAGGACGGCGGCCACTACGACGCTGAGGTCAAGACCACCTACAAGGCCAAGAAGCCCGTGCAGC
TGCCCGGCGCCTACAACGTCAACATCAAGTTGGACATCACCTCCCACAACGAGGACTACACCAT
CGTGGAACAGTACGAACGCGCCGAGGGCCGCCACTCCACCGGCGGCATGGACGAGCTGTACAAG
TAAGCGGCCGCGTCGAGTCTAGAGGGCCCGTTTAAACCCGCTGATCAGCCTCGACTGTGCCTTC
TAGTTGCCAGCCATCTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAGTAGGTGTCATTCTA
TTCTGGGGGGTGGGGTGGGGCAGGACAGCAAGGGGGAGGATTGGGAAGACAATAGCAGGCATGC
TGGGGATGCGGTGGGCTCTATGGATTTGGCTACAGCAACAGGGTGGTGGACCTCATGGCCCACA
TGGCCTCCAAGGAGTAAGACCCCTGGACCACCAGCCCCAGCAAGAGCACAAGAGGAAGAGAGAG
ACCCTCACTGCTGGGGAGTCCCTGCCACACTCAGTCCCCCACCACACTGAATCTCCCCTCCTCA
CAGTTGCCATGTAGACCCCTTGAAGAGGGGAGGGGCCTAGGGAGCCGCACCTTGTCATGTACCA
TCAATAAAGTACCCTGTGCTCAACCAGTTACTTGTCCTGTCTTATTCTAGGGTCTGGGGCAGAG
GGGAGGGAAGCTGGGCTTGTGTCAAGGTGAGACATTCTTGCTGGGGAGGGACCTGGTATGTTCT
CCTCAGACTGAGGGTAGGGCCTCCAAACAGCCTTGCTTGCTTCGAGAACCATTTGCTTCCCGCT
CAGACGTCTTGAGTGCTACAGGAAGCTGGCACCACTACTTCAGAGAACAAGGCCTTTTCCTCTC
CTCGCTCCAGT
exemplary donor template for insertion at GAPDH locus
SEQ ID NO: 41
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGGAGGGC
AGAGGAAGTCTTCTAACATGCGGTGACGTGGAGGAGAATCCTGGCCCGATGGTGAGCAAGGGCG
AGGAGGATAACATGGCCATCATCAAGGAGTTCATGCGCTTCAAGGTGCACATGGAGGGCTCCGT
GAACGGCCACGAGTTCGAGATCGAGGGCGAGGGCGAGGGCCGCCCCTACGAGGGCACCCAGACC
GCCAAGCTGAAGGTGACCAAGGGTGGCCCCCTGCCCTTCGCCTGGGACATCCTGTCCCCTCAGT
TCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTG
ACCCAGGACTCCTCCCTGCAGGACGGCGAGTTCATCTACAAGGTGAAGCTGCGCGGCACCAACT
TCCCCTCCGACGGCCCCGTAATGCAGAAGAAGACAATGGGCTGGGAGGCCTCCTCCGAGCGGAT
GTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCT
ACAACGTCAACATCAAGTTGGACATCACCTCCCACAACGAGGACTACACCATCGTGGAACAGTA
CGAACGCGCCGAGGGCCGCCACTCCACCGGCGGCATGGACGAGCTGTACAAGTAAGCGGCCGCG
TCGAGTCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGT
GGGCTCTATGGATTTGGCTACAGCAACAGGGTGGTGGACCTCATGGCCCACATGGCCTCCAAGG
AGTAAGACCCCTGGACCACCAGCCCCAGCAAGAGCACAAGAGGAAGAGAGAGACCCTCACTGCT
GGGGAGTCCCTGCCACACTCAGTCCCCCACCACACTGAATCTCCCCTCCTCACAGTTGCCATGT
AGACCCCTTGAAGAGGGGAGGGGCCTAGGGAGCCGCACCTTGTCATGTACCATCAATAAAGTAC
CCTGTGCTCAACCAGTTACTTGTCCTGTCTTATTCTAGGGTCTGGGGCAGAGGGGAGGGAAGCT
GGGCTTGTGTCAAGGTGAGACATTCTTGCTGGGGAGGGACCTGGTATGTTCTCCTCAGACTGAG
GGTAGGGCCTCCAAACAGCCTTGCTTGCTTCGAGAACCATTTGCTTCCCGCTCAGACGTCTTGA
GTGCTACAGGAAGCTGGCACCACTACTTCAGAGAACAAGGCCTTTTCCTCTCCTCGCTCCAGT
exemplary donor template for insertion at GAPDH locus
SEQ ID NO: 42
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGGGAAGCGGAGCTACTAAC
TTCAGCCTGCTGAAGCAGGCTGGAGACGTGGAGGAGAACCCTGGACCTATGGTGAGCAAGGGCG
AGGAGGATAACATGGCCATCATCAAGGAGTTCATGCGCTTCAAGGTGCACATGGAGGGCTCCGT
GAACGGCCACGAGTTCGAGATCGAGGGCGAGGGCGAGGGCCGCCCCTACGAGGGCACCCAGACC
GCCAAGCTGAAGGTGACCAAGGGTGGCCCCCTGCCCTTCGCCTGGGACATCCTGTCCCCTCAGT
TCATGTACGGCTCCAAGGCCTACGTGAAGCACCCCGCCGACATCCCCGACTACTTGAAGCTGTC
CTTCCCCGAGGGCTTCAAGTGGGAGCGCGTGATGAACTTCGAGGACGGCGGCGTGGTGACCGTG
ACCCAGGACTCCTCCCTGCAGGACGGCGAGTTCATCTACAAGGTGAAGCTGCGCGGCACCAACT
TCCCCTCCGACGGCCCCGTAATGCAGAAGAAGACAATGGGCTGGGAGGCCTCCTCCGAGCGGAT
GTACCCCGAGGACGGCGCCCTGAAGGGCGAGATCAAGCAGAGGCTGAAGCTGAAGGACGGCGGC
CACTACGACGCTGAGGTCAAGACCACCTACAAGGCCAAGAAGCCCGTGCAGCTGCCCGGCGCCT
ACAACGTCAACATCAAGTTGGACATCACCTCCCACAACGAGGACTACACCATCGTGGAACAGTA
CGAACGCGCCGAGGGCCGCCACTCCACCGGCGGCATGGACGAGCTGTACAAGTAAGCGGCCGCG
TCGAGTCTAGAGGGCCCGTTTAAACCCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTGTCCTT
TCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGT
GGGCTCTATGGATTTGGCTACAGCAACAGGGTGGTGGACCTCATGGCCCACATGGCCTCCAAGG
AGTAAGACCCCTGGACCACCAGCCCCAGCAAGAGCACAAGAGGAAGAGAGAGACCCTCACTGCT
GGGGAGTCCCTGCCACACTCAGTCCCCCACCACACTGAATCTCCCCTCCTCACAGTTGCCATGT
AGACCCCTTGAAGAGGGGAGGGGCCTAGGGAGCCGCACCTTGTCATGTACCATCAATAAAGTAC
CCTGTGCTCAACCAGTTACTTGTCCTGTCTTATTCTAGGGTCTGGGGCAGAGGGGAGGGAAGCT
GGGCTTGTGTCAAGGTGAGACATTCTTGCTGGGGAGGGACCTGGTATGTTCTCCTCAGACTGAG
GGTAGGGCCTCCAAACAGCCTTGCTTGCTTCGAGAACCATTTGCTTCCCGCTCAGACGTCTTGA
GTGCTACAGGAAGCTGGCACCACTACTTCAGAGAACAAGGCCTTTTCCTCTCCTCGCTCCAGT
exemplary donor template for insertion at GAPDH locus
SEQ ID NO: 43
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGAGGGCAGAGGAAGTCTTCTA
ACATGCGGTGACGTGGAGGAGAATCCTGGCCCGATGGTGAGCAAGGGCGAGGAGCTGTTCACCG
GGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAG
CTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGAC
CACTACCAGCAGAACACCCCCATCGGCGACGGCCCCGTGCTGCTGCCCGACAACCACTACCTGA
GCACCCAGTCCGCCCTGAGCAAAGACCCCAACGAGAAGCGCGATCACATGGTCCTGCTGGAGTT
CGTGACCGCCGCCGGGATCACTCTCGGCATGGACGAGCTGTACAAGTAACCCCTCTCCCTCCCC
CCCCCCTAACGTTACTGGCCGAAGCCGCTTGGAATAAGGCCGGTGTGCGTTTGTCTATATGTTA
TTTTCCACCATATTGCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTGTTGAATGTCGTGAA
GGAAGCAGTTCCTCTGGAAGCTTCTTGAAGACAAACAACGTCTGTAGCGACCCTTTGCAGGCAG
CGGAACCCCCCACCTGGCGACAGGTGCCTCTGCGGCCAAAAGCCACGTGTATAAGATACACCTG
CAAAGGCGGCACAACCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAAACGTCTAGGCC
CCCCGAACCACGGGGACGTGGTTTTCCTTTGAAAAACACGATGATAAATGGTGAGCAAGGGCGA
GGAGGATAACATGGCCATCATCAAGGAGTTCATGCGCTTCAAGGTGCACATGGAGGGCTCCGTG
AACGGCCACGAGTTCGAGATCGAGGGCGAGGGCGAGGGCCGCCCCTACGAGGGCACCCAGACCG
CCAAGCTGAAGGTGACCAAGGGTGGCCCCCTGCCCTTCGCCTGGGACATCCTGTCCCCTCAGTT
CATGTACGGCTCCAAGGCCTACGTGAAGCACCCCGCCGACATCCCCGACTACTTGAAGCTGTCC
TTCCCCGAGGGCTTCAAGTGGGAGCGCGTGATGAACTTCGAGGACGGCGGCGTGGTGACCGTGA
CCCAGGACTCCTCCCTGCAGGACGGCGAGTTCATCTACAAGGTGAAGCTGCGCGGCACCAACTT
CCCCTCCGACGGCCCCGTAATGCAGAAGAAGACAATGGGCTGGGAGGCCTCCTCCGAGCGGATG
TACCCCGAGGACGGCGCCCTGAAGGGCGAGATCAAGCAGAGGCTGAAGCTGAAGGACGGCGGCC
ACTACGACGCTGAGGTCAAGACCACCTACAAGGCCAAGAAGCCCGTGCAGCTGCCCGGCGCCTA
CAACGTCAACATCAAGTTGGACATCACCTCCCACAACGAGGACTACACCATCGTGGAACAGTAC
GAACGCGCCGAGGGCCGCCACTCCACCGGCGGCATGGACGAGCTGTACAAGTAAGCGGCCGCGT
CGAGTCTAGAGGGCCCGTTTAAACCCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGGGATGCGGTG
GGCTCTATGGATTTGGCTACAGCAACAGGGTGGTGGACCTCATGGCCCACATGGCCTCCAAGGA
GTAAGACCCCTGGACCACCAGCCCCAGCAAGAGCACAAGAGGAAGAGAGAGACCCTCACTGCTG
GGGAGTCCCTGCCACACTCAGTCCCCCACCACACTGAATCTCCCCTCCTCACAGTTGCCATGTA
GACCCCTTGAAGAGGGGGGGGCCTAGGGAGCCGCACCTTGTCATGTACCATCAATAAAGTACC
CTGTGCTCAACCAGTTACTTGTCCTGTCTTATTCTAGGGTCTGGGGCAGAGGGGAGGGAAGCTG
GGCTTGTGTCAAGGTGAGACATTCTTGCTGGGGAGGGACCTGGTATGTTCTCCTCAGACTGAGG
GTAGGGCCTCCAAACAGCCTTGCTTGCTTCGAGAACCATTTGCTTCCCGCTCAGACGTCTTGAG
TGCTACAGGAAGCTGGCACCACTACTTCAGAGAACAAGGCCTTTTCCTCTCCTCGCTCCAGT
exemplary donor template for insertion at GAPDH locus
SEQ ID NO: 44
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTGAGCAAGGGCGAGG
AGCTGTTCACCGGGGTGGTGCCCATCCTGGTCGAGCTGGACGGCGACGTAAACGGCCACAAGTT
CAGCGTGTCCGGCGAGGGCGAGGGCGATGCCACCTACGGCAAGCTGACCCTGAAGTTCATCTGC
ACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACG
GCAACATCCTGGGGCACAAGCTGGAGTACAACTACAACAGCCACAACGTCTATATCATGGCCGA
CAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACA
ACCACTACCTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAAGTGAGCG
GCCGCGTCGAGTCTAGAGGGCCCGTTTAAACCCGCTGATCAGCCTCGACTGTGCCTTCTAGTTG
CCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGA
TGCGGTGGGCTCTATGGATTTGGCTACAGCAACAGGGTGGTGGACCTCATGGCCCACATGGCCT
CCAAGGAGTAAGACCCCTGGACCACCAGCCCCAGCAAGAGCACAAGAGGAAGAGAGAGACCCTC
ACTGCTGGGGAGTCCCTGCCACACTCAGTCCCCCACCACACTGAATCTCCCCTCCTCACAGTTG
CCATGTAGACCCCTTGAAGAGGGGAGGGGCCTAGGGAGCCGCACCTTGTCATGTACCATCAATA
AAGTACCCTGTGCTCAACCAGTTACTTGTCCTGTCTTATTCTAGGGTCTGGGGCAGAGGGGAGG
GAAGCTGGGCTTGTGTCAAGGTGAGACATTCTTGCTGGGGAGGGACCTGGTATGTTCTCCTCAG
ACTGAGGGTAGGGCCTCCAAACAGCCTTGCTTGCTTCGAGAACCATTTGCTTCCCGCTCAGACG
TCTTGAGTGCTACAGGAAGCTGGCACCACTACTTCAGAGAACAAGGCCTTTTCCTCTCCTCGCT
CCAGT
exemplary donor template for insertion at GAPDH locus
SEQ ID NO: 45
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGACCTGATCCAGAGCATGCACATCGACGCCACACTGTACACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCTCTGGCG
GAGGAAGCGGAGGCGGAGGATCTGGTGGTGGTGGATCTGGCGGCGGTGGTAGTGGCGGAGGTTC
TCTGCAAATCACCTGTCCTCCACCTATGAGCGTGGAACACGCCGACATCTGGGTCAAGAGCTAC
AGCCTGTACAGCAGAGAGCGGTACATCTGCAACAGCGGCTTCAAGAGAAAGGCCGGCACAAGCA
GCCTGACCGAGTGTGTGCTGAACAAGGCCACAAACGTGGCCCACTGGACCACACCTAGCCTGAA
GTGCATCAGAGATCCCGCTCTGGTTCATCAGAGGCCTGCCCCTCCATCTACAGTGACAACAGCT
GGCGTGACCCCTCAGCCTGAGTCTCTGTCTCCATCTGGAAAAGAGCCTGCCGCCAGCTCTCCCA
GCTCTAACAATACTGCTGCCACCACAGCCGCTATCGTGCCTGGATCTCAGCTGATGCCTAGCAA
GAGCCCTAGCACCGGCACAACAGAGATCAGCTCTCACGAGAGCAGCCACGGAACACCTTCTCAG
ACCACCGCCAAGAATTGGGAGCTGACAGCCTCTGCCTCTCATCAGCCACCTGGCGTGTACCCAC
AGGGCCACTCTGATACAACAGTGGCCATCAGCACCAGCACCGTTCTGCTGTGTGGCCTGTCTGC
TGTTAGCCTGCTGGCCTGCTACCTGAAGTCTAGACAGACACCTCCTCTGGCCAGCGTGGAAATG
GAAGCCATGGAAGCTCTGCCTGTCACATGGGGCACCAGCAGCAGAGATGAGGACCTCGAGAATT
GCAGCCACCACCTGTAGGCGGCCGCGTCGAGTCTAGAGGGCCCGTTTAAACCCGCTGATCAGCC
TCGACTGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGGAGGATTGGGAAGAC
AATAGCAGGCATGCTGGGGATGCGGTGGGCTCTATGGATTTGGCTACAGCAACAGGGTGGTGGA
CCTCATGGCCCACATGGCCTCCAAGGAGTAAGACCCCTGGACCACCAGCCCCAGCAAGAGCACA
AGAGGAAGAGAGAGACCCTCACTGCTGGGGAGTCCCTGCCACACTCAGTCCCCCACCACACTGA
ATCTCCCCTCCTCACAGTTGCCATGTAGACCCCTTGAAGAGGGGAGGGGCCTAGGGAGCCGCAC
CTTGTCATGTACCATCAATAAAGTACCCTGTGCTCAACCAGTTACTTGTCCTGTCTTATTCTAG
GGTCTGGGGCAGAGGGGAGGGAAGCTGGGCTTGTGTCAAGGTGAGACATTCTTGCTGGGGAGGG
ACCTGGTATGTTCTCCTCAGACTGAGGGTAGGGCCTCCAAACAGCCTTGCTTGCTTCGAGAACC
ATTTGCTTCCCGCTCAGACGTCTTGAGTGCTACAGGAAGCTGGCACCACTACTTCAGAGAACAA
GGCCTTTTCCTCTCCTCGCTCCAGT
exemplary donor template for insertion at GAPDH locus
SEQ ID NO: 46
GGCTTTCCCATAATTTCCTTTCAAGGTGGGGAGGGAGGTAGAGGGGTGATGTGGGGAGTACGCT
GCAGGGCCTCACTCCTTTTGCAGACCACAGTCCATGCCATCACTGCCACCCAGAAGACTGTGGA
TGGCCCCTCCGGGAAACTGTGGCGTGATGGCCGCGGGGCTCTCCAGAACATCATCCCTGCCTCT
ACTGGCGCTGCCAAGGCTGTGGGCAAGGTCATCCCTGAGCTGAACGGGAAGCTCACTGGCATGG
CCTTCCGTGTCCCCACTGCCAACGTGTCAGTGGTGGACCTGACCTGCCGTCTAGAAAAACCTGC
CAAATATGATGACATCAAGAAGGTGGTGAAGCAGGCGTCGGAGGGCCCCCTCAAGGGCATCCTG
GGCTACACTGAGCACCAGGTGGTCTCCTCTGACTTCAACAGCGACACCCACTCCTCCACCTTTG
ACGCTGGGGCTGGCATTGCCCTCAACGACCACTTTGTCAAGCTCATCTCTTGGTACGACAATGA
GTTCGGATATAGCAATAGAGTGGTCGATCTGATGGCTCATATGGCTAGCAAAGAGGGAAGCGGA
GCTACTAACTTCAGCCTGCTGAAGCAGGCTGGAGACGTGGAGGAGAACCCTGGACCTATGGTGA
GCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGC
CATGCCCGAAGGCTACGTCCAGGAGCGCACCATCTTCTTCAAGGACGACGGCAACTACAAGACC
CGCGCCGAGGTGAAGTTCGAGGGCGACACCCTGGTGAACCGCATCGAGCTGAAGGGCATCGACT
TCAAGGAGGACGGCAACATCCTGGGGCACAAGCTGGAGTACAACTACAACAGCCACAACGTCTA
TATCATGGCCGACAAGCAGAAGAACGGCATCAAGGTGAACTTCAAGATCCGCCACAACATCGAG
GACGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAGCG
CGATCACATGGTCCTGCTGGAGTTCGTGACCGCCGCCGGGATCACTCTCGGCATGGACGAGCTG
TACAAGTGAGCGGCCGCGTCGAGTCTAGAGGGCCCGTTTAAACCCGCTGATCAGCCTCGACTGT
GCCTTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGGTGTC
ATTCTATTCTGGGGGGGGGGTGGGGCAGGACAGCAAGGGGGAGGATTGGGAAGACAATAGCAG
GCATGCTGGGGATGCGGTGGGCTCTATGGAGACTGGCTCTTAAAAAGTGCAGGGTCTGGCGCCC
TCTGGTGGCTGGCTCAGAAAAAGGGCCCTGACAACTCTTTTCATCTTCTAGGTATGACAACGAA
TTTGGCTACAGCAACAGGGTGGTGGACCTCATGGCCCACATGGCCTCCAAGGAGTAAGACCCCT
GGACCACCAGCCCCAGCAAGAGCACAAGAGGAAGAGAGAGACCCTCACTGCTGGGGAGTCCCTG
CCACACTCAGTCCCCCACCACACTGAATCTCCCCTCCTCACAGTTGCCATGTAGACCCCTTGAA
GAGGGGAGGGGCCTAGGGAGCCGCACCTTGTCATGTACCATCAATAAAGTACCCTGTGCTCAAC
CAGTTACTTGTCCTGTCTTATTCTAGGGTCTGGGGCAGAGGGGAGGGAAGCTGGGCTTGTGTCA
AGGTGAGACATTCTTGCTGGGGAGGGACCTGGTATGTTCTCCTCAGACTGAGGGTAGGGCCTCC
AAACAGCCTTGCTTGCT
exemplary donor template for insertion at TBP locus
SEQ ID NO: 47
GCAGACTTCCATTTACAGTGAGGAGGTGAGCATTGCATTGAACAAAAGATGGCGTTTTCACTTG
GAATTAGTTATCTGAAGCTTTAGGATTCCTCAGCAATATGATTATGAGACAAGAAAGGAAGATT
CAGAAATGAGTCTAGTTGAAGGCAGCAATTCAGAGAAGAAGATTCAGTTGTTATCATTGCCGTC
CTGCTTGGTTTATGGCCTGGTTCAGGACCAAGGAGAGAAGTGTGAATACATGCCTCTTGAGCTA
TAGAATGAGACGCTGGAGTCACTAAGATGATTTTTTAAAAGTATTGTTTTATAAACAAAAATAA
GATTGTGACAAGGGATTCCACTATTAATGTTTTCATGCCTGTGCCTTAATCTGACTGGGTATGG
TGAGAATTGTGCTTGCAGCTTTAAGGTAAGAATTTTACCATCTTAATATGTTAAGAAGTGCCAT
TTCAGTCTCTCATCTCTACTCCAACTTGTCTTCTTAGGTGCTAAAGTCAGAGCCGAAATCTACG
AGGCCTTCGAGAACATCTACCCCATCCTGAAGGGCTTCAGAAAGACCACCGGAAGCGGAGCTAC
TAACTTCAGCCTGCTGAAGCAGGCTGGAGACGTGGAGGAGAACCCTGGACCTATGGTGAGCAAG
GGCGAGGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAG
GAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGCTG
CCCGACAACCACTACCTGAGCACCCAGTCCGCCCTGAGCAAAGACCCCAACGAGAAGCGCGATC
ACATGGTCCTGCTGGAGTTCGTGACCGCCGCCGGGATCACTCTCGGCATGGACGAGCTGTACAA
GTGAGCGGCCGCGTCGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGCAGAAATTTATGAAGCATTTGAAAACATCTACCCTATTCT
AAAGGGATTCAGGAAGACGACGTAATGGCTCTCATGTACCCTTGCCTCCCCCACCCCCTTCTTT
TTTTTTTTTTAAACAAATCAGTTTGTTTTGGTACCTTTAAATGGTGGTGTTGTGAGAAGATGGA
TGTTGAGTTGCAGGGTGTGGCACCAGGTGATGCCCTTCTGTAAGTGCCCACCGCGGGATGCCGG
GAAGGGGCATTATTTGTGCACTGAGAACACCGCGCAGCGTGACTGTGAGTTGCTCATACCGTGC
TGCTATCTGGGCAGCGCTGCCCATTTATTTATATGTAGATTTTAAACACTGCTGTTGACAAGTT
GGTTTGAGGGAGAAAACTTTAAGTGTTAAAGCCACCTCTATAATTGATTGGACTTTTTAATTTT
AATGTTTTTCCCCATGAACCACAGTTTTTATATTTCTACCAGAAAAGTAAAAATCTTTTTTAAA
AGTGTTGTTTTT
exemplary donor template for insertion at TBP locus
SEQ ID NO: 49
CTGACCACAGCTCTGCAAGCAGACTTCCATTTACAGTGAGGAGGTGAGCATTGCATTGAACAAA
AGATGGCGTTTTCACTTGGAATTAGTTATCTGAAGCTTTAGGATTCCTCAGCAATATGATTATG
AGACAAGAAAGGAAGATTCAGAAATGAGTCTAGTTGAAGGCAGCAATTCAGAGAAGAAGATTCA
GTTGTTATCATTGCCGTCCTGCTTGGTTTATGGCCTGGTTCAGGACCAAGGAGAGAAGTGTGAA
TACATGCCTCTTGAGCTATAGAATGAGACGCTGGAGTCACTAAGATGATTTTTTAAAAGTATTG
TTTTATAAACAAAAATAAGATTGTGACAAGGGATTCCACTATTAATGTTTTCATGCCTGTGCCT
TAATCTGACTGGGTATGGTGAGAATTGTGCTTGCAGCTTTAAGGTAAGAATTTTACCATCTTAA
TATGTTAAGAAGTGCCATTTCAGTCTCTCATCTCTACTCCAACTTGTCTTCTTAGGGGCTAAAG
TGCGGGCCGAGATCTACGAGGCCTTCGAGAATATCTACCCCATCCTGAAGGGCTTCAGAAAGAC
CACCGGAAGCGGAGCTACTAACTTCAGCCTGCTGAAGCAGGCTGGAGACGTGGAGGAGAACCCT
GGACCTATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACGG
CAAGCTGACCCTGAAGTTCATCTGCACCACCGGCAAGCTGCCCGTGCCCTGGCCCACCCTCGTG
ACCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACGG
CAACTACAAGACCCGCGCCGAGGTGAAGTTCGAGGGCGACACCCTGGTGAACCGCATCGAGCTG
AAGGGCATCGACTTCAAGGAGGACGGCAACATCCTGGGGCACAAGCTGGAGTACAACTACAACA
GCCACAACGTCTATATCATGGCCGACAAGCAGAAGAACGGCATCAAGGTGAACTTCAAGATCCG
CCACAACATCGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGATCACTCTCGG
CATGGACGAGCTGTACAAGTGAGCGGCCGCGTCGAGTCTAGAGGGCCCGTTTAAACCCGCTGAT
CAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGT
CTGAGTAGGTGTCATTCTATTCTGGGGGGTGGGGTGGGGCAGGACAGCAAGGGGGAGGATTGGG
AAGACAATAGCAGGCATGCTGGGGATGCGGTGGGCTCTATGGTAGGTGCTAAAGTCAGAGCAGA
AATTTATGAAGCATTTGAAAACATCTACCCTATTCTAAAGGGATTCAGGAAGACGACGTAATGG
CTCTCATGTACCCTTGCCTCCCCCACCCCCTTCTTTTTTTTTTTTTAAACAAATCAGTTTGTTT
TGGTACCTTTAAATGGTGGTGTTGTGAGAAGATGGATGTTGAGTTGCAGGGTGTGGCACCAGGT
GATGCCCTTCTGTAAGTGCCCACCGCGGGATGCCGGGAAGGGGCATTATTTGTGCACTGAGAAC
ACCGCGCAGCGTGACTGTGAGTTGCTCATACCGTGCTGCTATCTGGGCAGCGCTGCCCATTTAT
TTATATGTAGATTTTAAACACTGCTGTTGACAAGTTGGTTTGAGGGAGAAAACTTTAAGTGTTA
AAGCCACCTCTATAATTGATTGGACTTTTTAATTTTAATGTTTTTCCCCATGAACCACAGTTTT
TATATTTCTACCAGAAAAGTAAAAATCTTT
exemplary donor template for insertion at TBP locus
SEQ ID NO: 50
ACAAAAGATGGCGTTTTCACTTGGAATTAGTTATCTGAAGCTTTAGGATTCCTCAGCAATATGA
TTATGAGACAAGAAAGGAAGATTCAGAAATGAGTCTAGTTGAAGGCAGCAATTCAGAGAAGAAG
ATTCAGTTGTTATCATTGCCGTCCTGCTTGGTTTATGGCCTGGTTCAGGACCAAGGAGAGAAGT
GTGAATACATGCCTCTTGAGCTATAGAATGAGACGCTGGAGTCACTAAGATGATTTTTTAAAAG
TATTGTTTTATAAACAAAAATAAGATTGTGACAAGGGATTCCACTATTAATGTTTTCATGCCTG
TGCCTTAATCTGACTGGGTATGGTGAGAATTGTGCTTGCAGCTTTAAGGTAAGAATTTTACCAT
CTTAATATGTTAAGAAGTGCCATTTCAGTCTCTCATCTCTACTCCAACTTGTCTTCTTAGGTGC
TAAAGTCAGAGCAGAAATTTATGAAGCATTCGAGAACATCTACCCTATTCTAAAGGGATTCAGG
AAGACGACGGGAAGCGGAGCTACTAACTTCAGCCTGCTGAAGCAGGCTGGAGACGTGGAGGAGA
ACCCTGGACCTATGGTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGTGCCCTGGCCCACCC
TCGTGACCACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTCAAGGAC
GACGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAACTA
CAACAGCCACAACGTCTATATCATGGCCGACAAGCAGAAGAACGGCATCAAGGTGAACTTCAAG
ATCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCGGGATCACT
CTCGGCATGGACGAGCTGTACAAGTGAGCGGCCGCGTCGAGTCTAGAGGGCCCGTTTAAACCCG
CTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGAAGGGATTCAGGAAGAC
GACGTAATGGCTCTCATGTACCCTTGCCTCCCCCACCCCCTTCTTTTTTTTTTTTTAAACAAAT
CAGTTTGTTTTGGTACCTTTAAATGGTGGTGTTGTGAGAAGATGGATGTTGAGTTGCAGGGTGT
GGCACCAGGTGATGCCCTTCTGTAAGTGCCCACCGCGGGATGCCGGGAAGGGGCATTATTTGTG
CACTGAGAACACCGCGCAGCGTGACTGTGAGTTGCTCATACCGTGCTGCTATCTGGGCAGCGCT
GCCCATTTATTTATATGTAGATTTTAAACACTGCTGTTGACAAGTTGGTTTGAGGGAGAAAACT
TTAAGTGTTAAAGCCACCTCTATAATTGATTGGACTTTTTAATTTTAATGTTTTTCCCCATGAA
CCACAGTTTTTATATTTCTACCAGAAAAGTAAAAATCTTTTTTAAAAGTGTTGTTTTTCTAATT
TATAACTCCTAGGGGTTATTTCTGTGCCAGACACA
exemplary donor template for insertion at G6PD locus
SEQ ID NO: 51
GGCCCGGGGGACTCCACATGGTGGCAGGCAGTGGCATCAGCAAGACACTCTCTCCCTCACAGAA
CGTGAAGCTCCCTGACGCCTATGAGCGCCTCATCCTGGACGTCTTCTGCGGGAGCCAGATGCAC
TTCGTGCGCAGGTGAGGCCCAGCTGCCGGCCCCTGCATACCTGTGGGCTATGGGGTGGCCTTTG
CCCTCCCTCCCTGTGTGCCACCGGCCTCCCAAGCCATACCATGTCCCCTCAGCGACGAGCTCCG
TGAGGCCTGGCGTATTTTCACCCCACTGCTGCACCAGATTGAGCTGGAGAAGCCCAAGCCCATC
CCCTATATTTATGGCAGGTGAGGAAAGGGTGGGGGCTGGGGACAGAGCCCAGCGGGCAGGGGCG
GGGTGAGGGTGGAGCTACCTCATGCCTCTCCTCCACCCGTCACTCTCCAGCCGAGGCCCCACGG
AGGCAGACGAGCTGATGAAGAGAGTGGGCTTCCAGTACGAGGGAACCTACAAATGGGTCAACCC
TCACAAGCTGGGAAGCGGAGCTACTAACTTCAGCCTGCTGAAGCAGGCTGGAGACGTGGAGGAG
AACCCTGGACCTATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCAGC
ACGACTTCTTCAAGTCCGCCATGCCCGAAGGCTACGTCCAGGAGCGCACCATCTTCTTCAAGGA
CGACGGCAACTACAAGACCCGCGCCGAGGTGAAGTTCGAGGGCGACACCCTGGTGAACCGCATC
GAGCTGAAGGGCATCGACTTCAAGGAGGACGGCAACATCCTGGGGCACAAGCTGGAGTACAACT
ACAACAGCCACAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCAGAACACCCCC
ATCGGCGACGGCCCCGTGCTGCTGCCCGACAACCACTACCTGAGCACCCAGTCCGCCCTGAGCA
AAGACCCCAACGAGAAGCGCGATCACATGGTCCTGCTGGAGTTCGTGACCGCCGCCGGGATCAC
TCTCGGCATGGACGAGCTGTACAAGTGAGCGGCCGCGTCGAGTCTAGAGGGCCCGTTTAAACCC
GCTGATCAGCCTCGACTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGAGG
ATTGGGAAGACAATAGCAGGCATGCTGGGGATGCGGTGGGCTCTATGGGTGGGTGAACCCCCAC
AAGCTCTGAGCCCTGGGCACCCACCTCCACCCCCGCCACGGCCACCCTCCTTCCCGCCGCCCGA
CCCCGAGTCGGGAGGACTCCGGGACCATTGACCTCAGCTGCACATTCCTGGCCCCGGGCTCTGG
CCACCCTGGCCCGCCCCTCGCTGCTGCTACTACCCGAGCCCAGCTACATTCCTCAGCTGCCAAG
CACTCGAGACCATCCTGGCCCCTCCAGACCCTGCCTGAGCCCAGGAGCTGAGTCACCTCCTCCA
CTCACTCCAGCCCAACAGAAGGAAGGAGGAGGGCGCCCATTCGTCTGTCCCAGAGCTTATTGGC
CACTGGGTCTCACTCCTGAGTGGGGCCAGGGTGGGAGGGAGGGACGAGGGGGAGGAAAGGGGCG
AGCACCCACGTGAGAGAATCTGCCTGTGGCCTTGCCCGCCAGCCTCAGTGCCACTTGACATTCC
TTGTCACCAGCAACATCTCGAGCCCCCTGGATGTCC
exemplary donor template for insertion at E2F4 locus
SEQ ID NO: 52
CCAGGGGGCTGTAGTGGGGCCAGGCTGGACCTCTGTGCCCTGAGCATGGCTTTCTTGTTTTTCA
GTTTTGGAACTCCCCAAAGAGCTGTCAGAAATCTTTGATCCCACACGAGGTAGGCTGCTGCATT
CCTCCCTGAGGCTAGGGGTAAGGGACACAGCTCATTGGGTCCTATGGCTGTTTTCTTGCCCTTT
TGAGGACCTTGTTGTGGCGCTTATGGTAACTGGGGCAAAGGGTGAAGTTCCTGATGGGCAGGTG
GGGTTCCCTTTCCTGGGCTTTGGTGGGTGGAGAGGTGGGAGCTGGAATGTTAGTAACTGAGCTC
CCTCCATTCCCAGAGTGCATGAGCTCGGAGCTGCTGGAGGAGTTGATGTCCTCAGAAGGTGGGT
GGCCCTGGAAGGTGGGAGTGGGTGTGGGCAGGGGTTGGGCTGCTGCTAGGGGAGCCCTGGCCCA
GGGCCTGAGACTAGTGCTCTCTGCAGTGTTCGCCCCTCTGCTGAGACTTTCTCCTCCTCCTGGC
GACCACGACTACATCTACAACCTGGACGAGAGCGAGGGCGTGTGCGACCTGTTTGATGTGCCCG
TGCTGAACCTGGGAAGCGGAGCTACTAACTTCAGCCTGCTGAAGCAGGCTGGAGACGTGGAGGA
GAACCCTGGACCTATGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCTTCTTCAAGG
ACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGTGAACTTCA
AGATCCGCCACAACATCGAGGACGGCAGCGTGCAGCTCGCCGACCACTACCAGCAGAACACCCC
CATCGGCGACGGCCCCGTGCTGCTGCCCGACAACCACTACCTGAGCACCCAGTCCGCCCTGAGC
AAAGACCCCAACGAGAAGCGCGATCACATGGTCCTGCTGGAGTTCGTGACCGCCGCCGGGATCA
CTCTCGGCATGGACGAGCTGTACAAGTGAGCGGCCGCGTCGAGTCTAGAGGGCCCGTTTAAACC
CGCTGATCAGCCTCGACTGTGCCTTCTAGTTGCCAGCCATCTGTTGTTTGCCCCTCCCCCGTGC
CTTCCTTGACCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGCCACCCCCGGGAGAC
CACGATTATATCTACAACCTGGACGAGAGTGAAGGTGTCTGTGACCTCTTTGATGTGCCTGTTC
TCAACCTCTGACTGACAGGGACATGCCCTGTGTGGCTGGGACCCAGACTGTCTGACCTGGGGGT
TGCCTGGGGACCTCTCCCACCCGACCCCTACAGAGCTTGAGAGCCACAGACGCCTGGCTTCTCC
GGCCTCCCCTCACCGCACAGTTCTGGCCACAGCTCCCGCTCCTGTGCTGGCACTTCTGTGCTCG
CAGAGCAGGGGAACAGGACTCAGCCCCCATCACCGTGGAGCCAAAGTGTTTGCTTCTCCCTTTC
TGCGGCCTTCGCCAGCCCAGGCTCGGCTGCCACCCAGTGGCACAGAACCGAGGAGCTGCCATTA
CCCCCCATAGGGGGCAGTGTCTTGTTCCTGCCAGCCTCAGTGTCTTGCTTCTGCCAGCTCCTTC
CCCTAGGAGGGAAGGGTGGGGTGGAACTGGGCACATG
exemplary donor template for insertion at E2F4 locus
SEQ ID NO: 53
CCAGGCTGGACCTCTGTGCCCTGAGCATGGCTTTCTTGTTTTTCAGTTTTGGAACTCCCCAAAG
AGCTGTCAGAAATCTTTGATCCCACACGAGGTAGGCTGCTGCATTCCTCCCTGAGGCTAGGGGT
AAGGGACACAGCTCATTGGGTCCTATGGCTGTTTTCTTGCCCTTTTGAGGACCTTGTTGTGGCG
CTTATGGTAACTGGGGCAAAGGGTGAAGTTCCTGATGGGCAGGTGGGGTTCCCTTTCCTGGGCT
TTGGTGGGTGGAGAGGTGGGAGCTGGAATGTTAGTAACTGAGCTCCCTCCATTCCCAGAGTGCA
TGAGCTCGGAGCTGCTGGAGGAGTTGATGTCCTCAGAAGGTGGGTGGCCCTGGAAGGTGGGAGT
GGGTGTGGGCAGGGGTTGGGCTGCTGCTAGGGGAGCCCTGGCCCAGGGCCTGAGACTAGTGCTC
TCTGCAGTGTTTGCCCCTCTGCTTCGTCTTAGTCCTCCTCCGGGCGACCACGACTACATCTACA
ACCTGGACGAGAGCGAGGGCGTGTGCGACCTGTTTGATGTGCCCGTGCTGAACCTGGGAAGCGG
AGCTACTAACTTCAGCCTGCTGAAGCAGGCTGGAGACGTGGAGGAGAACCCTGGACCTATGGTG
AGCAAGGGCGAGGAGCTGTTCACCGGGGTGGTGCCCATCCTGGTCGAGCTGGACGGCGACGTAA
ACGGCCACAAGTTCAGCGTGTCCGGCGAGGGCGAGGGCGATGCCACCTACGGCAAGCTGACCCT
GAAGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCCACAACGTCT
ATATCATGGCCGACAAGCAGAAGAACGGCATCAAGGTGAACTTCAAGATCCGCCACAACATCGA
GGACGGCAGCGTGCAGCTCGCCGACCACTACCAGCAGAACACCCCCATCGGCGACGGCCCCGTG
CTGCTGCCCGACAACCACTACCTGAGCACCCAGTCCGCCCTGAGCAAAGACCCCAACGAGAAGC
GCGATCACATGGTCCTGCTGGAGTTCGTGACCGCCGCCGGGATCACTCTCGGCATGGACGAGCT
GTACAAGTGAGCGGCCGCGTCGAGTCTAGAGGGCCCGTTTAAACCCGCTGATCAGCCTCGACTG
TGCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCA
GGCATGCTGGGGATGCGGTGGGCTCTATGGATTATATCTACAACCTGGACGAGAGTGAAGGTGT
CTGTGACCTCTTTGATGTGCCTGTTCTCAACCTCTGACTGACAGGGACATGCCCTGTGTGGCTG
GGACCCAGACTGTCTGACCTGGGGGTTGCCTGGGGACCTCTCCCACCCGACCCCTACAGAGCTT
GAGAGCCACAGACGCCTGGCTTCTCCGGCCTCCCCTCACCGCACAGTTCTGGCCACAGCTCCCG
CTCCTGTGCTGGCACTTCTGTGCTCGCAGAGCAGGGGAACAGGACTCAGCCCCCATCACCGTGG
AGCCAAAGTGTTTGCTTCTCCCTTTCTGCGGCCTTCGCCAGCCCAGGCTCGGCTGCCACCCAGT
GGCACAGAACCGAGGAGCTGCCATTACCCCCCATAGGGGGCAGTGTCTTGTTCCTGCCAGCCTC
AGTGTCTTGCTTCTGCCAGCTCCTTCCCCTAGGAGGGAAGGGTGGGGTGGAACTGGGCACATGC
CAGCACCACTTCTAGCTT
exemplary donor template for insertion at E2F4 locus
SEQ ID NO: 54
GTCAGAAATCTTTGATCCCACACGAGGTAGGCTGCTGCATTCCTCCCTGAGGCTAGGGGTAAGG
GACACAGCTCATTGGGTCCTATGGCTGTTTTCTTGCCCTTTTGAGGACCTTGTTGTGGCGCTTA
TGGTAACTGGGGCAAAGGGTGAAGTTCCTGATGGGCAGGTGGGGTTCCCTTTCCTGGGCTTTGG
TGGGTGGAGAGGTGGGAGCTGGAATGTTAGTAACTGAGCTCCCTCCATTCCCAGAGTGCATGAG
CTCGGAGCTGCTGGAGGAGTTGATGTCCTCAGAAGGTGGGTGGCCCTGGAAGGTGGGAGTGGGT
GTGGGCAGGGGTTGGGCTGCTGCTAGGGGAGCCCTGGCCCAGGGCCTGAGACTAGTGCTCTCTG
CAGTGTTTGCCCCTCTGCTTCGTCTTTCTCCACCCCCGGGAGACCACGATTATATCTACAACCT
GGACGAGAGTGAAGGTGTCTGTGACCTCTTCGACGTGCCCGTGCTCAACCTCGGAAGCGGAGCT
ACTAACTTCAGCCTGCTGAAGCAGGCTGGAGACGTGGAGGAGAACCCTGGACCTATGGTGAGCA
AGGGCGAGGAGCTGTTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCAAGCTGACCCTGAAG
TTCATCTGCACCACCGGCAAGCTGCCCGTGCCCTGGCCCACCCTCGTGACCACCCTGACCTACG
GCGTGCAGTGCTTCAGCCGCTACCCCGACCACATGAAGCAGCACGACTTCTTCAAGTCCGCCAT
GCCCGAAGGCTACGTCCAGGAGCGCACCATCTTCTTCAAGGACGACGGCAACTACAAGACCCGC
GCCGAGGTGAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAACGTCTATAT
CATGGCCGACAAGCAGAAGAACGGCATCAAGGTGAACTTCAAGATCCGCCACAACATCGAGGAC
GGCAGCGTGCAGCTCGCCGACCACTACCAGCAGAACACCCCCATCGGCGACGGCCCCGTGCTGC
TGCCCGACAACCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGACGAGCTGTAC
AAGTGAGCGGCCGCGTCGAGTCTAGAGGGCCCGTTTAAACCCGCTGATCAGCCTCGACTGTGCC
TTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCA
TGCTGGGGATGCGGTGGGCTCTATGGTGACTGACAGGGACATGCCCTGTGTGGCTGGGACCCAG
ACTGTCTGACCTGGGGGTTGCCTGGGGACCTCTCCCACCCGACCCCTACAGAGCTTGAGAGCCA
CAGACGCCTGGCTTCTCCGGCCTCCCCTCACCGCACAGTTCTGGCCACAGCTCCCGCTCCTGTG
CTGGCACTTCTGTGCTCGCAGAGCAGGGGAACAGGACTCAGCCCCCATCACCGTGGAGCCAAAG
TGTTTGCTTCTCCCTTTCTGCGGCCTTCGCCAGCCCAGGCTCGGCTGCCACCCAGTGGCACAGA
ACCGAGGAGCTGCCATTACCCCCCATAGGGGGCAGTGTCTTGTTCCTGCCAGCCTCAGTGTCTT
GCTTCTGCCAGCTCCTTCCCCTAGGAGGGAAGGGTGGGGTGGAACTGGGCACATGCCAGCACCA
CTTCTAGCTTCCTTCGCTATCCCCCACCCCCTGACCCTCCAGCTCCTCCTGGCCCTCTCACGTG
CCCACTTCTGCTGG
exemplary donor template for insertion at KIF11 locus
SEQ ID NO: 55
AGAGCAGGGTTTCTTGACAGCAGTGCTATTGGCATTTTAAACTGGATAATTCTTTGTTGTGATG
GGCTTTCCTGTGGACTGTACTATGTTGGTACACAAGAAAAACAGTGTACTATGTGAATACTCAC
TCAAAGCCAGTAGCACTCCCTGATTGTAACACCAAAAAAGTCTCTCAGCATTGCCAAATGTCCC
CTGTGGCAGCAGAATCACTCCCTGATGAGAACCACTACCCTGGAGTAAAATCTATAACTATGTC
TTAGAAAATAACACAGAAAATTAATATTTCTTTCACTCTACTCCTTCCATTAGTGATCAAATAA
AGAAGGCATTTGGCGCTACTTGCCAAATTGTTGGCTCAAACTTGTGCTGAACCTTTTTTGGTTT
TCTACACTTAAGTTTTTTTGCCTATAACCCAGAGAACTTTGAAAATAGAGTGTAGTTAATGTGT
ATCTAATGTTACTTTGTATTGACTTAATTTACCGGCCTTTAATCCACAGCATAAGAAGTCCCAC
GGCAAGGACAAAGAGAACCGGGGCATCAACACACTGGAACGGTCCAAGGTCGAGGAAACAACCG
AGCACCTGGTCACCAAGAGCAGACTGCCTCTGAGAGCCCAGATCAACCTGGGAAGCGGAGCTAC
TAACTTCAGCCTGCTGAAGCAGGCTGGAGACGTGGAGGAGAACCCTGGACCTATGGTGAGCAAG
GGCGAGGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAG
GAGGACGGCAACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCCCGTGCTGCTG
CCCGACAACCACTACCTGAGCACCCAGTCCGCCCTGAGCAAAGACCCCAACGAGAAGCGCGATC
ACATGGTCCTGCTGGAGTTCGTGACCGCCGCCGGGATCACTCTCGGCATGGACGAGCTGTACAA
GTGAGCGGCCGCGTCGAGTCTAGAGGGCCCGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGGCATG
CTGGGGATGCGGTGGGCTCTATGGAAAAAATCACATGGAAAAGACAAAGAAAACAGAGGCATTA
ACACACTGGAGAGGTCTAAAGTGGAAGAAACTACAGAGCACTTGGTTACAAAGAGCAGATTACC
TCTGCGAGCCCAGATCAACCTTTAATTCACTTGGGGGTTGGCAATTTTATTTTTAAAGAAAACT
TAAAAATAAAACCTGAAACCCCAGAACTTGAGCCTTGTGTATAGATTTTAAAAGAATATATATA
TCAGCCGGGCGCGGTGGCTCATGCCTGTAATCCCAGCACTTTGGGAGGCTGAGGCGGGTGGATT
GCTTGAGCCCAGGAGTTTGAGACCAGCCTGGCCAACGTGGCAAAACCTCGTCTCTGTTAAAAAT
TAGCCGGGCGTGGTGGCACACTCCTGTAATCCCAGCTACTGGGGAGGCTGAGGCACGAGAATCA
CTTGAACCCAGGAAGCGGGGTTGCAGTGAGCCAAAGGTACACCACTACACTCCAGCCTGGGCAA
CAGAGCAAGACT
exemplary donor template for insertion at KIF11 locus
SEQ ID NO: 56
TTCCTGTGGACTGTACTATGTTGGTACACAAGAAAAACAGTGTACTATGTGAATACTCACTCAA
AGCCAGTAGCACTCCCTGATTGTAACACCAAAAAAGTCTCTCAGCATTGCCAAATGTCCCCTGT
GGCAGCAGAATCACTCCCTGATGAGAACCACTACCCTGGAGTAAAATCTATAACTATGTCTTAG
AAAATAACACAGAAAATTAATATTTCTTTCACTCTACTCCTTCCATTAGTGATCAAATAAAGAA
GGCATTTGGCGCTACTTGCCAAATTGTTGGCTCAAACTTGTGCTGAACCTTTTTTGGTTTTCTA
CACTTAAGTTTTTTTGCCTATAACCCAGAGAACTTTGAAAATAGAGTGTAGTTAATGTGTATCT
AATGTTACTTTGTATTGACTTAATTTTCCCGCCTTAAATCCACAGCATAAAAAATCACATGGAA
AAGACAAAGAAAACAGAGGCATTAACACACTGGAGAGGTCTAAAGTGGAAGAAACAACCGAGCA
CCTGGTCACCAAGAGCAGACTGCCTCTGAGAGCCCAGATCAACCTGGGAAGCGGAGCTACTAAC
TTCAGCCTGCTGAAGCAGGCTGGAGACGTGGAGGAGAACCCTGGACCTATGGTGAGCAAGGGCG
AGGAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGG
ACGGCAACATCCTGGGGCACAAGCTGGAGTACAACTACAACAGCCACAACGTCTATATCATGGC
CGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGCGATCACAT
GGTCCTGCTGGAGTTCGTGACCGCCGCCGGGATCACTCTCGGCATGGACGAGCTGTACAAGTGA
GCGGCCGCGTCGAGTCTAGAGGGCCCGTTTAAACCCGCTGATCAGCCTCGACTGTGCCTTCTAG
TTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGACAATAGCAGGCATGCTGG
GGATGCGGTGGGCTCTATGGAACTACAGAGCACTTGGCTACATAGAGCAGATTACCTCTGCGAG
CCCAGATCAACCTTTAATTCACTTGGGGGTTGGCAATTTTATTTTTAAAGAAAACTTAAAAATA
AAACCTGAAACCCCAGAACTTGAGCCTTGTGTATAGATTTTAAAAGAATATATATATCAGCCGG
GCGCGGTGGCTCATGCCTGTAATCCCAGCACTTTGGGAGGCTGAGGCGGGTGGATTGCTTGAGC
CCAGGAGTTTGAGACCAGCCTGGCCAACGTGGCAAAACCTCGTCTCTGTTAAAAATTAGCCGGG
CGTGGTGGCACACTCCTGTAATCCCAGCTACTGGGGAGGCTGAGGCACGAGAATCACTTGAACC
CAGGAAGCGGGGTTGCAGTGAGCCAAAGGTACACCACTACACTCCAGCCTGGGCAACAGAGCAA
GACTCGGTCTCAAAAACAAAATTTAAAAAAGATATAAGGCAGTACTGTAAATTCAGTTGAATTT
TGATATCT
exemplary donor template for insertion at KIF11 locus
SEQ ID NO: 57
TTAAACTGGATAATTCTTTGTTGTGATGGGCTTTCCTGTGGACTGTACTATGTTGGTACACAAG
AAAAACAGTGTACTATGTGAATACTCACTCAAAGCCAGTAGCACTCCCTGATTGTAACACCAAA
AAAGTCTCTCAGCATTGCCAAATGTCCCCTGTGGCAGCAGAATCACTCCCTGATGAGAACCACT
ACCCTGGAGTAAAATCTATAACTATGTCTTAGAAAATAACACAGAAAATTAATATTTCTTTCAC
TCTACTCCTTCCATTAGTGATCAAATAAAGAAGGCATTTGGCGCTACTTGCCAAATTGTTGGCT
CAAACTTGTGCTGAACCTTTTTTGGTTTTCTACACTTAAGTTTTTTTGCCTATAACCCAGAGAA
CTTTGAAAATAGAGTGTAGTTAATGTGTATCTAATGTTACTTTGTATTGACTTAATTTTCCCGC
CTTAAATCCACAGCATAAAAAATCACATGGAAAAGACAAAGAAAACAGAGGCATCAACACACTG
GAACGGTCCAAGGTCGAGGAAACAACCGAGCACCTGGTCACCAAGAGCAGACTGCCTCTGAGAG
CCCAGATCAACCTGGGAAGCGGAGCTACTAACTTCAGCCTGCTGAAGCAGGCTGGAGACGTGGA
GGAGAACCCTGGACCTATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGAGGGCGAGGGCGATG
CCACCTACGGCAAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCGACCACATGAAG
CAGCACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAACCG
CATCGAGCTGAAGGGCATCGACTTCAAGGAGGACGGCAACATCCTGGGGCACAAGCTGGAGTAC
AACTACAACAGCCACAACGTCTATATCATGGCCGACAAGCAGAAGAACGGCATCAAGGTGAACT
TCAAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCCTG
AGCAAAGACCCCAACGAGAAGCGCGATCACATGGTCCTGCTGGAGTTCGTGACCGCCGCCGGGA
TCACTCTCGGCATGGACGAGCTGTACAAGTGAGCGGCCGCGTCGAGTCTAGAGGGCCCGTTTAA
ACCCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGATTAACACACTG
GAGAGTTCTGAAGTGGAAGAAACTACAGAGCACTTGGTTACAAAGAGCAGATTACCTCTGCGAG
CCCAGATCAACCTTTAATTCACTTGGGGGTTGGCAATTTTATTTTTAAAGAAAACTTAAAAATA
AAACCTGAAACCCCAGAACTTGAGCCTTGTGTATAGATTTTAAAAGAATATATATATCAGCCGG
GCGCGGTGGCTCATGCCTGTAATCCCAGCACTTTGGGAGGCTGAGGCGGGTGGATTGCTTGAGC
CCAGGAGTTTGAGACCAGCCTGGCCAACGTGGCAAAACCTCGTCTCTGTTAAAAATTAGCCGGG
CGTGGTGGCACACTCCTGTAATCCCAGCTACTGGGGAGGCTGAGGCACGAGAATCACTTGAACC
CAGGAAGCGGGGTTGCAGTGAGCCAAAGGTACACCACTACACTCCAGCCTGGGCAACAGAGCAA
GACTCGGTCTCAAAAACAAAATTTAAAAAAGATATAAGGC
exemplary donor template for insertion at GAPDH locus
SEQ ID NO: 48
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCCCCTGGTAGCGG
CGCTGTTGCTGGGCTCGGCGTGCTGCGGATCAGCTCAGCTACTATTTAATAAAACAAAATCTGT
AGAATTCACGTTTTGTAATGACACTGTCGTCATTCCATGCTTTGTTACTAATATGGAGGCACAA
AACACTACTGAAGTATACGTAAAGTGGAAATTTAAAGGAAGAGATATTTACACCTTTGATGGAG
CTCTAAACAAGTCCACTGTCCCCACTGACTTTAGTAGTGCAAAAATTGAAGTCTCACAATTACT
AAAAGGAGATGCCTCTTTGAAGATGGATAAGAGTGATGCTGTCTCACACACAGGAAACTACACT
TGTGAAGTAACAGAATTAACCAGAGAAGGTGAAACGATCATCGAGCTAAAATATCGTGTTGTTT
CATGGTTTTCTCCAAATGAAAATATTCTTATTGTTATTTTCCCAATTTTTGCTATACTCCTGTT
CTGGGGACAGTTTGGTATTAAAACACTTAAATATAGATCCGGTGGTATGGATGAGAAAACAATT
GCTTTACTTGTTGCTGGACTAGTGATCACTGTCATTGTCATTGTTGGAGCCATTCTTTTCGTCC
CAGGTGAATATTCATTAAAGAATGCTACTGGCCTTGGTTTAATTGTGACTTCTACAGGGATATT
AATATTACTTCACTACTATGTGTTTAGTACAGCGATTGGATTAACCTCCTTCGTCATTGCCATA
TTGGTTATTCAGGTGATAGCCTATATCCTCGCTGTGGTTGGACTGAGTCTCTGTATTGCGGCGT
GTATACCAATGCATGGCCCTCTTCTGATTTCAGGTTTGAGTATCTTAGCTCTAGCACAATTACT
TGGACTAGTTTATATGAAATTTGTGGCTTCCAATCAGAAGACTATACAACCTCCTAGGAAAGCT
GTAGAGGAACCCCTTAATGCATTCAAAGAATCAAAAGGAATGATGAATGATGAATGAGCGGCCG
CGTCGAGTCTAGAGGGCCCGTTTAAACCCGCTGATCAGCCTCGACTGTGCCTTCTAGTTGCCAG
CCATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGG
TGGGGTGGGGCAGGACAGCAAGGGGGAGGATTGGGAAGACAATAGCAGGCATGCTGGGGATGCG
GTGGGCTCTATGGATTTGGCTACAGCAACAGGGTGGTGGACCTCATGGCCCACATGGCCTCCAA
GGAGTAAGACCCCTGGACCACCAGCCCCAGCAAGAGCACAAGAGGAAGAGAGAGACCCTCACTG
CTGGGGAGTCCCTGCCACACTCAGTCCCCCACCACACTGAATCTCCCCTCCTCACAGTTGCCAT
GTAGACCCCTTGAAGAGGGGAGGGGCCTAGGGAGCCGCACCTTGTCATGTACCATCAATAAAGT
ACCCTGTGCTCAACCAGTTACTTGTCCTGTCTTATTCTAGGGTCTGGGGCAGAGGGGAGGGAAG
CTGGGCTTGTGTCAAGGTGAGACATTCTTGCTGGGGAGGGACCTGGTATGTTCTCCTCAGACTG
AGGGTAGGGCCTCCAAACAGCCTTGCTTGCTTCGAGAACCATTTGCTTCCCGCTCAGACGTCTT
GAGTGCTACAGGAAGCTGGCACCACTACTTCAGAGAACAAGGCCTTTTCCTCTCCTCGCTCCAG
T
exemplary donor template for insertion at GAPDH locus
SEQ ID NO: 205
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAACTGCTGCTGC
CTACAGCTCTGCTGCTTCTGGTGTCTGCCGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGTAAGCGGCCGCGTCGAGTCTAGAGG
GCCCGTTTAAACCCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAATG
AGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGAT
TTGGCTACAGCAACAGGGTGGTGGACCTCATGGCCCACATGGCCTCCAAGGAGTAAGACCCCTG
GACCACCAGCCCCAGCAAGAGCACAAGAGGAAGAGAGAGACCCTCACTGCTGGGGAGTCCCTGC
CACACTCAGTCCCCCACCACACTGAATCTCCCCTCCTCACAGTTGCCATGTAGACCCCTTGAAG
AGGGGAGGGGCCTAGGGAGCCGCACCTTGTCATGTACCATCAATAAAGTACCCTGTGCTCAACC
AGTTACTTGTCCTGTCTTATTCTAGGGTCTGGGGCAGAGGGGAGGGAAGCTGGGCTTGTGTCAA
GGTGAGACATTCTTGCTGGGGAGGGACCTGGTATGTTCTCCTCAGACTGAGGGTAGGGCCTCCA
AACAGCCTTGCTTGCTTCGAGAACCATTTGCTTCCCGCTCAGACGTCTTGAGTGCTACAGGAAG
CTGGCACCACTACTTCAGAGAACAAGGCCTTTTCCTCTCCTCGCTCCAGT
exemplary donor template for insertion at GAPDH locus
SEQ ID NO: 206
GTCGACGAAGACTGTGGATGGCCCCTCCGGGAAACTGTGGCGTGATGGCCGCGGGGCTCTCCAG
AACATCATCCCTGCCTCTACTGGCGCTGCCAAGGCTGTGGGCAAGGTCATCCCTGAGCTGAACG
GGAAGCTCACTGGCATGGCCTTCCGTGTCCCCACTGCCAACGTGTCAGTGGTGGACCTGACCTG
CCGTCTAGAAAAACCTGCCAAATATGATGACATCAAGAAGGTGGTGAAGCAGGCGTCGGAGGGC
CCCCTCAAGGGCATCCTGGGCTACACTGAGCACCAGGTGGTCTCCTCTGACTTCAACAGCGACA
CCCACTCCTCCACCTTTGACGCTGGGGCTGGCATTGCCCTCAACGACCACTTTGTCAAGCTCAT
TTCCTGGTATGTGGCTGGGGCCAGAGACTGGCTCTTAAAAAGTGCAGGGTCTGGCGCCCTCTGG
TGGCTGGCTCAGAAAAAGGGCCCTGACAACTCTTTACATCTTCTAGGTATGACAACGAGTTCGG
ATATAGCAATAGAGTGGTCGATCTGATGGCTCATATGGCTAGCAAAGAGGGAAGCGGAGCTACT
AACTTCAGCCTGCTGAAGCAGGCTGGAGACGTGGAGGAGAACCCTGGACCTATGCTTCTCCTGG
TGACAAGCCTTCTGCTCTGTGAGTTACCACACCCAGCATTCCTCCTGATCCCAGACATCCAGAT
GACACAGACTACATCCTCCCTGTCTGCCTCTCTGGGAGACAGAGTCACCATCAGTTGCAGGGCA
AGTCAGGACATTAGTAAATATTTAAATTGGTATCAGCAGAAACCAGATGGAACTGTTAAACTCC
TGATCTACCATACATCAAGATTACACTCAGGAGTCCCATCAAGGTTCAGTGGCAGTGGGTCTGG
AACAGATTATTCTCTCACCATTAGCAACCTGGAGCAAGAAGATATTGCCACTTACTTTTGCCAA
CAGGGTAATACGCTTCCGTACACGTTCGGAGGGGGGACTAAGTTGGAAATAACAGGCTCCACCT
CTGGATCCGGCAAGCCCGGATCTGGCGAGGGATCCACCAAGGGCGAGGTGAAACTGCAGGAGTC
AGGACCTGGCCTGGTGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCTGGAGTGGCTGGGAG
TAATATGGGGTAGTGAAACCACATACTATAATTCAGCTCTCAAATCCAGACTGACCATCATCAA
GGACAACTCCAAGAGCCAAGTTTTCTTAAAAATGAACAGTCTGCAAACTGATGACACAGCCATT
TACTACTGTGCCAAACATTATTACTACGGTGGTAGCTATGCTATGGACTACTGGGGTCAAGGAA
CCTCAGTCACCGTCTCCTCAGCGGCCGCAATTGAAGTTATGTATCCTCCTCCTTACCTAGACAA
TGAGAAGAGCAATGGAACCATTATCCATGTGAAAGGGAAACACCTTTGTCCAAGTCCCCTATTT
CCCGGACCTTCTAAGCCCTTTTGGGTGCTGGTGGTGGTTGGGGGAGTCCTGGCTTGCTATAGCT
TGCTAGTAACAGTGGCCTTTATTATTTTCTGGGTGAGGAGTAAGAGGAGCAGGCTCCTGCACAG
TGACTACATGAACATGACTCCCCGCCGCCCCGGGCCCACCCGCAAGCATTACCAGCCCTATGCC
CCACCACGCGACTTCGCAGCCTATCGCTCCAGAGTGAAGTTCAGCAGGAGCGCAGACGCCCCCG
CGTACCAGCAGGGCCAGAACCAGCTCTATAACGAGCTCAATCTAGGACGAAGAGAGGAGTACGA
TGTTTTGGACAAGAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGTGAGATTGGGA
TGAAAGGCGAGCGCCGGAGGGGCAAGGGGCACGATGGCCTTTACCAGGGTCTCAGTACAGCCAC
CAAGGACACCTACGACGCCCTTCACATGCAGGCCCTGCCCCCTCGCTAAAGCGGCCGCGTCGAG
TCTAGAGGGCCCGTTTAAACCCGCTGATCAGCCTCGACTGTGCCTTCTAGTTGCCAGCCATCTG
TTGTTTGCCCCTCCCCCGTGCCTTCCTTGACCCTGGAAGGTGCCACTCCCACTGTCCTTTCCTA
ATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGCGGTGGGCT
CTATGGATTTGGCTACAGCAACAGGGTGGTGGACCTCATGGCCCACATGGCCTCCAAGGAGTAA
GACCCCTGGACCACCAGCCCCAGCAAGAGCACAAGAGGAAGAGAGAGACCCTCACTGCTGGGGA
GTCCCTGCCACACTCAGTCCCCCACCACACTGAATCTCCCCTCCTCACAGTTGCCATGTAGACC
CCTTGAAGAGGGGAGGGGCCTAGGGAGCCGCACCTTGTCATGTACCATCAATAAAGTACCCTGT
GCTCAACCAGTTACTTGTCCTGTCTTATTCTAGGGTCTGGGGCAGAGGGGAGGGAAGCTGGGCT
TGTGTCAAGGTGAGACATTCTTGCTGGGGAGGGACCTGGTATGTTCTCCTCAGACTGAGGGTAG
GGCCTCCAAACAGCCTTGCTTGCTTCGAGAACCATTTGCTTCCCGCTCAGACGTCTTGAGTGCT
ACAGGAAGCTGGCACCACTACTTCAGAGAACAAGGCCTTTTCCTCTCCTCGCTCCAGT
exemplary donor template for insertion at GAPDH locus
SEQ ID NO: 207
GTCGACGAAGACTGTGGATGGCCCCTCCGGGAAACTGTGGCGTGATGGCCGCGGGGCTCTCCAG
AACATCATCCCTGCCTCTACTGGCGCTGCCAAGGCTGTGGGCAAGGTCATCCCTGAGCTGAACG
GGAAGCTCACTGGCATGGCCTTCCGTGTCCCCACTGCCAACGTGTCAGTGGTGGACCTGACCTG
CCGTCTAGAAAAACCTGCCAAATATGATGACATCAAGAAGGTGGTGAAGCAGGCGTCGGAGGGC
CCCCTCAAGGGCATCCTGGGCTACACTGAGCACCAGGTGGTCTCCTCTGACTTCAACAGCGACA
CCCACTCCTCCACCTTTGACGCTGGGGCTGGCATTGCCCTCAACGACCACTTTGTCAAGCTCAT
TTCCTGGTATGTGGCTGGGGCCAGAGACTGGCTCTTAAAAAGTGCAGGGTCTGGCGCCCTCTGG
TGGCTGGCTCAGAAAAAGGGCCCTGACAACTCTTTACATCTTCTAGGTATGACAACGAGTTCGG
ATATAGCAATAGAGTGGTCGATCTGATGGCTCATATGGCTAGCAAAGAGGGAAGCGGAGCTACT
AACTTCAGCCTGCTGAAGCAGGCTGGAGACGTGGAGGAGAACCCTGGACCTATGGCACTCCCCG
TCACCGCCCTTCTCTTGCCCCTCGCCCTGCTGCTGCATGCTGCCAGGCCCATGGACGAAGTGCA
GCTCGTGGAGTCCGGTGGAGGACTCGTCCAACCGGGCGGATCCCTTCGCTTGTCCTGCGCCGCA
TCAGGCTTCAGCTTCACCAACTATGGCGTCCACTGGGTCAGACAGGCCCCCGGAAAGGGACTGG
AATGGGTGTCCGTGATCTGGAGCGGCGGGAACACCGACTACAACACCTCCGTGAAGGGCCGGTT
CACTATTAGCCGCGACAACTCCAAGAACACTCTGTACCTCCAAATGAACTCCCTGAGGGCCGAA
GATACTGCTGTGTACTATTGCGCGAGAGCCCTGACCTACTACGACTACGAGTTCGCGTACTGGG
GCCAGGGGACTCTCGTGACCGTGTCCAGCGGTGGTGGAGGTTCCGGAGGCGGAGGTTCTGGTGG
CGGGGGATCAGAAATCGTGCTGACTCAGTCCCCTGCGACCTTGTCCCTGAGCCCTGGAGAACGG
GCCACCCTGAGCTGTAGAGCCAGCCAGAGCATCGGGACAAATATTCACTGGTACCAGCAGAAAC
CCGGACAAGCACCACGGCTGCTGATCTACTACGCCTCCGAGTCGATTTCCGGAATCCCGGCTCG
CTTTTCGGGGTCTGGATCGGGAACGGACTTCACTCTGACCATCTCGTCGCTGGAACCCGAGGAT
TTCGCCGTGTACTACTGCCAACAGAACAACAATTGGCCGACCACGTTCGGCCAGGGCACCAAGC
TCGAGATTAAGGGATCACTGGAAGCGGCCGCAACCACAACACCTGCTCCAAGGCCCCCCACACC
CGCTCCAACTATAGCCAGCCAACCATTGAGCCTCAGACCTGAAGCTTGCAGGCCCGCAGCAGGA
GGCGCCGTCCATACGCGAGGCCTGGACTTCGCGTGTGATATTTATATTTGGGCCCCTTTGGCCG
GAACATGTGGGGTGTTGCTTCTCTCCCTTGTGATCACTCTGTATTGTAAGCGCGGGAGAAAGAA
GCTCCTGTACATCTTCAAGCAGCCTTTTATGCGACCTGTGCAAACCACTCAGGAAGAAGATGGG
TGTTCATGCCGCTTCCCCGAGGAGGAAGAAGGAGGGTGTGAACTGAGGGTGAAATTTTCTAGAA
GCGCCGATGCTCCCGCATATCAGCAGGGTCAGAATCAGCTCTACAATGAATTGAATCTCGGCAG
GCGAGAAGAGTACGATGTTCTGGACAAGAGACGGGGCAGGGATCCCGAGATGGGGGGAAAGCCC
CGGAGAAAAAATCCTCAGGAGGGGTTGTACAATGAGCTGCAGAAGGACAAGATGGCTGAAGCCT
ATAGCGAGATCGGAATGAAAGGCGAAAGACGCAGAGGCAAGGGGCATGACGGTCTGTACCAGGG
TCTCTCTACAGCCACCAAGGACACTTATGATGCGTTGCATATGCAAGCCTTGCCACCCCGCTAA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 208
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGACCTGATCCAGAGCATGCACATCGACGCCACACTGTACACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCGGAAGCG
GAGCCACAAACTTCTCTCTGCTGAAGCAGGCAGGAGATGTTGAAGAAAACCCTGGACCTATCAC
CTGTCCTCCACCTATGAGCGTGGAACACGCCGACATCTGGGTCAAGAGCTACAGCCTGTACAGC
AGAGAGCGGTACATCTGCAACAGCGGCTTCAAGAGAAAGGCCGGCACAAGCAGCCTGACCGAGT
GTGTGCTGAACAAGGCCACAAACGTGGCCCACTGGACCACACCTAGCCTGAAGTGCATCAGAGA
TCCCGCTCTGGTTCATCAGAGGCCTGCCCCTCCATCTACAGTGACAACAGCTGGCGTGACCCCT
CAGCCTGAGTCTCTGTCTCCATCTGGAAAAGAGCCTGCCGCCAGCTCTCCCAGCTCTAACAATA
CTGCTGCCACCACAGCCGCTATCGTGCCTGGATCTCAGCTGATGCCTAGCAAGAGCCCTAGCAC
CGGCACAACAGAGATCAGCTCTCACGAGAGCAGCCACGGAACACCTTCTCAGACCACCGCCAAG
AATTGGGAGCTGACAGCCTCTGCCTCTCATCAGCCACCTGGCGTGTACCCACAGGGCCACTCTG
ATACAACAGTGGCCATCAGCACCAGCACCGTTCTGCTGTGTGGCCTGTCTGCTGTTAGCCTGCT
GGCCTGCTACCTGAAGTCTAGACAGACACCTCCTCTGGCCAGCGTGGAAATGGAAGCCATGGAA
GCTCTGCCTGTCACATGGGGCACCAGCAGCAGAGATGAGGACCTCGAGAATTGCAGCCACCACC
TGGGAAGCGGAGCCACAAACTTCTCTCTGCTGAAGCAGGCAGGAGATGTTGAAGAAAACCCTGG
ACCTATGGTGAGCAAGGGCGAGGAGCTGTTCACCGGGGTGGTGCCCATCCTGGTCGAGCTGGAC
GGCGACGTAAACGGCCACAAGTTCAGCGTGTCCGGCGAGGGCGAGGGCGATGCCACCTACGGCA
AGCTGACCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCACGACTTC
TTCAAGTCCGCCATGCCCGAAGGCTACGTCCAGGAGCGCACCATCTTCTTCAAGGACGACGGCA
ACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGC
CACAACGTCTATATCATGGCCGACAAGCAGAAGAACGGCATCAAGGTGAACTTCAAGATCCGCC
ACAACATCGAGGACGGCAGCGTGCAGCTCGCCGACCACTACCAGCAGAACACCCCCATCGGCGA
CGGCCCCGTGCTGCTGCCCGACAACCACTACCTGAGCACCCAGTCCGCCCTGAGCAAAGACCCC
AACGAGAAGCGCGATCACATGGTCCTGCTGGAGTTCGTGACCGCCGCCGGGATCACTCTCGGCA
TGGACGAGCTGTACAAGTAAGCGGCCGCGTCGAGTCTAGAGGGCCCGTTTAAACCCGCTGATCA
GCCTCGACTGTGCCTTCTAGTTGCCAGCCATCTGTTGTTTGCCCCTCCCCCGTGCCTTCCTTGA
CCCTGGAAGGTGCCACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGGGGAGGATTGGGAA
GACAATAGCAGGCATGCTGGGGATGCGGTGGGCTCTATGGATTTGGCTACAGCAACAGGGTGGT
GGACCTCATGGCCCACATGGCCTCCAAGGAGTAAGACCCCTGGACCACCAGCCCCAGCAAGAGC
ACAAGAGGAAGAGAGAGACCCTCACTGCTGGGGAGTCCCTGCCACACTCAGTCCCCCACCACAC
TGAATCTCCCCTCCTCACAGTTGCCATGTAGACCCCTTGAAGAGGGGAGGGGCCTAGGGAGCCG
CACCTTGTCATGTACCATCAATAAAGTACCCTGTGCTCAACCAGTTACTTGTCCTGTCTTATTC
TAGGGTCTGGGGCAGAGGGGAGGGAAGCTGGGCTTGTGTCAAGGTGAGACATTCTTGCTGGGGA
GGGACCTGGTATGTTCTCCTCAGACTGAGGGTAGGGCCTCCAAACAGCCTTGCTTGCTTCGAGA
ACCATTTGCTTCCCGCTCAGACGTCTTGAGTGCTACAGGAAGCTGGCACCACTACTTCAGAGAA
CAAGGCCTTTTCCTCTCCTCGCTCCAGT
exemplary donor template for insertion at GAPDH locus
SEQ ID NO: 209
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCGGAAGCGGAGC
CACAAACTTCTCTCTGCTGAAGCAGGCAGGAGATGTTGAAGAAAACCCTGGACCTATCACCTGT
CCTCCACCTATGAGCGTGGAACACGCCGACATCTGGGTCAAGAGCTACAGCCTGTACAGCAGAG
AGCGGTACATCTGCAACAGCGGCTTCAAGAGAAAGGCCGGCACAAGCAGCCTGACCGAGTGTGT
GCTGAACAAGGCCACAAACGTGGCCCACTGGACCACACCTAGCCTGAAGTGCATCAGAGATCCC
GCTCTGGTTCATCAGAGGCCTGCCCCTCCATCTACAGTGACAACAGCTGGCGTGACCCCTCAGC
CTGAGTCTCTGTCTCCATCTGGAAAAGAGCCTGCCGCCAGCTCTCCCAGCTCTAACAATACTGC
TGCCACCACAGCCGCTATCGTGCCTGGATCTCAGCTGATGCCTAGCAAGAGCCCTAGCACCGGC
ACAACAGAGATCAGCTCTCACGAGAGCAGCCACGGAACACCTTCTCAGACCACCGCCAAGAATT
GGGAGCTGACAGCCTCTGCCTCTCATCAGCCACCTGGCGTGTACCCACAGGGCCACTCTGATAC
AACAGTGGCCATCAGCACCAGCACCGTTCTGCTGTGTGGCCTGTCTGCTGTTAGCCTGCTGGCC
TGCTACCTGAAGTCTAGACAGACACCTCCTCTGGCCAGCGTGGAAATGGAAGCCATGGAAGCTC
TGCCTGTCACATGGGGCACCAGCAGCAGAGATGAGGACCTCGAGAATTGCAGCCACCACCTGTA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 210
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGACCTGATCCAGAGCATGCACATCGACGCCACACTGTACACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCTCTGGCG
GAGGAAGCGGAGGCGGAGGATCTGGTGGTGGTGGATCTGGCGGCGGTGGTAGTGGCGGAGGTTC
TCTGCAAATCACCTGTCCTCCACCTATGAGCGTGGAACACGCCGACATCTGGGTCAAGAGCTAC
AGCCTGTACAGCAGAGAGCGGTACATCTGCAACAGCGGCTTCAAGAGAAAGGCCGGCACAAGCA
GCCTGACCGAGTGTGTGCTGAACAAGGCCACAAACGTGGCCCACTGGACCACACCTAGCCTGAA
GTGCATCAGAGATCCCGCTCTGGTTCATCAGAGGCCTGCCCCTCCATCTACAGTGACAACAGCT
GGCGTGACCCCTCAGCCTGAGTCTCTGTCTCCATCTGGAAAAGAGCCTGCCGCCAGCTCTCCCA
GCTCTAACAATACTGCTGCCACCACAGCCGCTATCGTGCCTGGATCTCAGCTGATGCCTAGCAA
GAGCCCTAGCACCGGCACAACAGAGATCAGCTCTCACGAGAGCAGCCACGGAACACCTTCTCAG
ACCACCGCCAAGAATTGGGAGCTGACAGCCTCTGCCTCTCATCAGCCACCTGGCGTGTACCCAC
AGGGCCACTCTGATACAACAGTGGCCATCAGCACCAGCACCGTTCTGCTGTGTGGCCTGTCTGC
TGTTAGCCTGCTGGCCTGCTACCTGAAGTCTAGACAGACACCTCCTCTGGCCAGCGTGGAAATG
GAAGCCATGGAAGCTCTGCCTGTCACATGGGGCACCAGCAGCAGAGATGAGGACCTCGAGAATT
GCAGCCACCACCTGGGAAGCGGAGCCACAAACTTCTCTCTGCTGAAGCAGGCAGGAGATGTTGA
AGAAAACCCTGGACCTATGTGGCAGCTGTTGCTGCCGACAGCCCTCCTGTTGCTGGTCTCCGCT
GGCATGAGAACCGAGGATCTGCCTAAGGCCGTGGTGTTCCTGGAACCTCAGTGGTACAGAGTGC
TGGAAAAGGACAGCGTGACCCTGAAGTGCCAGGGCGCCTATTCTCCCGAGGACAATAGCACCCA
GTGGTTCCACAACGAGAGCCTGATCAGCAGCCAGGCCAGCAGCTACTTTATCGATGCCGCCACC
GTGGACGACAGCGGCGAGTACAGATGCCAGACCAATCTGAGCACCCTGAGCGACCCTGTGCAGC
TGGAAGTGCACATTGGATGGTTGCTGCTGCAAGCCCCTAGATGGGTGTTCAAAGAAGAGGACCC
CATCCACCTGAGATGCCACTCTTGGAAGAACACAGCCCTGCACAAAGTGACCTACCTGCAGAAC
GGCAAGGGCAGAAAGTACTTCCACCACAACAGCGACTTCTACATCCCCAAGGCCACACTGAAGG
ACTCCGGCTCCTACTTCTGCAGAGGCCTGGTCGGCAGCAAGAACGTGTCCAGCGAGACAGTGAA
CATCACCATCACACAGGGCCTCGCCGTGTCTACCATCAGCAGCTTTTTCCCACCTGGCTATCAG
GTGTCCTTCTGCCTGGTCATGGTGCTGCTGTTCGCCGTGGATACCGGCCTGTACTTCAGCGTCA
AGACCAACATCCGGTCCAGCACCAGAGACTGGAAGGACCACAAGTTCAAGTGGCGGAAGGACCC
TCAGGACAAGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 211
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTGGACCTGGATCC
TGTTTCTGGTGGCCGCTGCCACAAGAGTGCACAGCAATTGGGTCAACGTGATCAGCGACCTGAA
GAAGATCGAGGACCTGATCCAGAGCATGCACATCGACGCCACACTGTACACCGAGTCCGATGTG
CACCCTAGCTGCAAAGTGACCGCCATGAAGTGCTTTCTGCTGGAACTGCAAGTGATCAGCCTGG
AAAGCGGCGACGCCAGCATCCACGATACCGTGGAAAACCTGATCATCCTGGCCAACAACAGCCT
GAGCAGCAACGGCAATGTGACCGAGAGCGGCTGCAAAGAGTGCGAGGAACTGGAAGAGAAGAAC
ATCAAAGAGTTCCTCCAGAGCTTCGTCCACATCGTGCAGATGTTCATCAACACCAGCGGAAGCG
GAGCCACAAACTTCTCTCTGCTGAAGCAGGCAGGAGATGTTGAAGAAAACCCTGGACCTATCAC
CTGTCCTCCACCTATGAGCGTGGAACACGCCGACATCTGGGTCAAGAGCTACAGCCTGTACAGC
AGAGAGCGGTACATCTGCAACAGCGGCTTCAAGAGAAAGGCCGGCACAAGCAGCCTGACCGAGT
GTGTGCTGAACAAGGCCACAAACGTGGCCCACTGGACCACACCTAGCCTGAAGTGCATCAGAGA
TCCCGCTCTGGTTCATCAGAGGCCTGCCCCTCCATCTACAGTGACAACAGCTGGCGTGACCCCT
CAGCCTGAGTCTCTGTCTCCATCTGGAAAAGAGCCTGCCGCCAGCTCTCCCAGCTCTAACAATA
CTGCTGCCACCACAGCCGCTATCGTGCCTGGATCTCAGCTGATGCCTAGCAAGAGCCCTAGCAC
CGGCACAACAGAGATCAGCTCTCACGAGAGCAGCCACGGAACACCTTCTCAGACCACCGCCAAG
AATTGGGAGCTGACAGCCTCTGCCTCTCATCAGCCACCTGGCGTGTACCCACAGGGCCACTCTG
ATACAACAGTGGCCATCAGCACCAGCACCGTTCTGCTGTGTGGCCTGTCTGCTGTTAGCCTGCT
GGCCTGCTACCTGAAGTCTAGACAGACACCTCCTCTGGCCAGCGTGGAAATGGAAGCCATGGAA
GCTCTGCCTGTCACATGGGGCACCAGCAGCAGAGATGAGGACCTCGAGAATTGCAGCCACCACC
TGGGAAGCGGAGCCACAAACTTCTCTCTGCTGAAGCAGGCAGGAGATGTTGAAGAAAACCCTGG
ACCTATGTGGCAGCTGTTGCTGCCGACAGCCCTCCTGTTGCTGGTCTCCGCTGGCATGAGAACC
GAGGATCTGCCTAAGGCCGTGGTGTTCCTGGAACCTCAGTGGTACAGAGTGCTGGAAAAGGACA
GCGTGACCCTGAAGTGCCAGGGCGCCTATTCTCCCGAGGACAATAGCACCCAGTGGTTCCACAA
CGAGAGCCTGATCAGCAGCCAGGCCAGCAGCTACTTTATCGATGCCGCCACCGTGGACGACAGC
GGCGAGTACAGATGCCAGACCAATCTGAGCACCCTGAGCGACCCTGTGCAGCTGGAAGTGCACA
TTGGATGGTTGCTGCTGCAAGCCCCTAGATGGGTGTTCAAAGAAGAGGACCCCATCCACCTGAG
ATGCCACTCTTGGAAGAACACAGCCCTGCACAAAGTGACCTACCTGCAGAACGGCAAGGGCAGA
AAGTACTTCCACCACAACAGCGACTTCTACATCCCCAAGGCCACACTGAAGGACTCCGGCTCCT
ACTTCTGCAGAGGCCTGGTCGGCAGCAAGAACGTGTCCAGCGAGACAGTGAACATCACCATCAC
ACAGGGCCTCGCCGTGTCTACCATCAGCAGCTTTTTCCCACCTGGCTATCAGGTGTCCTTCTGC
CTGGTCATGGTGCTGCTGTTCGCCGTGGATACCGGCCTGTACTTCAGCGTCAAGACCAACATCC
GGTCCAGCACCAGAGACTGGAAGGACCACAAGTTCAAGTGGCGGAAGGACCCTCAGGACAAGTA
AGCGGCCGCGTCGAGTCTAGAGGGCCCGTTTAAACCCGCTGATCAGCCTCGACTGTGCCTTCTA
GTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTG
GGGATGCGGTGGGCTCTATGGATTTGGCTACAGCAACAGGGTGGTGGACCTCATGGCCCACATG
GCCTCCAAGGAGTAAGACCCCTGGACCACCAGCCCCAGCAAGAGCACAAGAGGAAGAGAGAGAC
CCTCACTGCTGGGGAGTCCCTGCCACACTCAGTCCCCCACCACACTGAATCTCCCCTCCTCACA
GTTGCCATGTAGACCCCTTGAAGAGGGGAGGGGCCTAGGGAGCCGCACCTTGTCATGTACCATC
AATAAAGTACCCTGTGCTCAACCAGTTACTTGTCCTGTCTTATTCTAGGGTCTGGGGCAGAGGG
GAGGGAAGCTGGGCTTGTGTCAAGGTGAGACATTCTTGCTGGGGAGGGACCTGGTATGTTCTCC
TCAGACTGAGGGTAGGGCCTCCAAACAGCCTTGCTTGCTTCGAGAACCATTTGCTTCCCGCTCA
GACGTCTTGAGTGCTACAGGAAGCTGGCACCACTACTTCAGAGAACAAGGCCTTTTCCTCTCCT
CGCTCCAGT
exemplary donor template for insertion at GAPDH locus
SEQ ID NO: 212
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCTCTGGCGGAGG
AAGCGGAGGCGGAGGATCTGGTGGTGGTGGATCTGGCGGCGGTGGTAGTGGCGGAGGTTCTCTG
CAAATCACCTGTCCTCCACCTATGAGCGTGGAACACGCCGACATCTGGGTCAAGAGCTACAGCC
TGTACAGCAGAGAGCGGTACATCTGCAACAGCGGCTTCAAGAGAAAGGCCGGCACAAGCAGCCT
GACCGAGTGTGTGCTGAACAAGGCCACAAACGTGGCCCACTGGACCACACCTAGCCTGAAGTGC
ATCAGAGATCCCGCTCTGGTTCATCAGAGGCCTGCCCCTCCATCTACAGTGACAACAGCTGGCG
TGACCCCTCAGCCTGAGTCTCTGTCTCCATCTGGAAAAGAGCCTGCCGCCAGCTCTCCCAGCTC
TAACAATACTGCTGCCACCACAGCCGCTATCGTGCCTGGATCTCAGCTGATGCCTAGCAAGAGC
CCTAGCACCGGCACAACAGAGATCAGCTCTCACGAGAGCAGCCACGGAACACCTTCTCAGACCA
CCGCCAAGAATTGGGAGCTGACAGCCTCTGCCTCTCATCAGCCACCTGGCGTGTACCCACAGGG
CCACTCTGATACAACAGTGGCCATCAGCACCAGCACCGTTCTGCTGTGTGGCCTGTCTGCTGTT
AGCCTGCTGGCCTGCTACCTGAAGTCTAGACAGACACCTCCTCTGGCCAGCGTGGAAATGGAAG
CCATGGAAGCTCTGCCTGTCACATGGGGCACCAGCAGCAGAGATGAGGACCTCGAGAATTGCAG
CCACCACCTGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 213
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTGGCAGCTGTTGCTGC
CGACAGCCCTCCTGTTGCTGGTCTCCGCTGGCATGAGAACCGAGGATCTGCCTAAGGCCGTGGT
GTTCCTGGAACCTCAGTGGTACAGAGTGCTGGAAAAGGACAGCGTGACCCTGAAGTGCCAGGGC
GCCTATTCTCCCGAGGACAATAGCACCCAGTGGTTCCACAACGAGAGCCTGATCAGCAGCCAGG
CCAGCAGCTACTTTATCGATGCCGCCACCGTGGACGACAGCGGCGAGTACAGATGCCAGACCAA
TCTGAGCACCCTGAGCGACCCTGTGCAGCTGGAAGTGCACATTGGATGGTTGCTGCTGCAAGCC
CCTAGATGGGTGTTCAAAGAAGAGGACCCCATCCACCTGAGATGCCACTCTTGGAAGAACACAG
CCCTGCACAAAGTGACCTACCTGCAGAACGGCAAGGGCAGAAAGTACTTCCACCACAACAGCGA
CTTCTACATCCCCAAGGCCACACTGAAGGACTCCGGCTCCTACTTCTGCAGAGGCCTGGTCGGC
AGCAAGAACGTGTCCAGCGAGACAGTGAACATCACCATCACACAGGGCCTCGCCGTGTCTACCA
TCAGCAGCTTTTTCCCACCTGGCTATCAGGTGTCCTTCTGCCTGGTCATGGTGCTGCTGTTCGC
CGTGGATACCGGCCTGTACTTCAGCGTCAAGACCAACATCCGGTCCAGCACCAGAGACTGGAAG
GACCACAAGTTCAAGTGGCGGAAGGACCCTCAGGACAAGGGAAGCGGAGCCACAAACTTCTCTC
TGCTGAAGCAGGCAGGAGATGTTGAAGAAAACCCTGGACCTATGGATTGGACCTGGATCCTGTT
TCTGGTGGCCGCTGCCACAAGAGTGCACAGCAATTGGGTCAACGTGATCAGCGACCTGAAGAAG
ATCGAGGACCTGATCCAGAGCATGCACATCGACGCCACACTGTACACCGAGTCCGATGTGCACC
CTAGCTGCAAAGTGACCGCCATGAAGTGCTTTCTGCTGGAACTGCAAGTGATCAGCCTGGAAAG
CGGCGACGCCAGCATCCACGATACCGTGGAAAACCTGATCATCCTGGCCAACAACAGCCTGAGC
AGCAACGGCAATGTGACCGAGAGCGGCTGCAAAGAGTGCGAGGAACTGGAAGAGAAGAACATCA
AAGAGTTCCTCCAGAGCTTCGTCCACATCGTGCAGATGTTCATCAACACCAGCTCTGGCGGAGG
AAGCGGAGGCGGAGGATCTGGTGGTGGTGGATCTGGCGGCGGTGGTAGTGGCGGAGGTTCTCTG
CAAATCACCTGTCCTCCACCTATGAGCGTGGAACACGCCGACATCTGGGTCAAGAGCTACAGCC
TGTACAGCAGAGAGCGGTACATCTGCAACAGCGGCTTCAAGAGAAAGGCCGGCACAAGCAGCCT
GACCGAGTGTGTGCTGAACAAGGCCACAAACGTGGCCCACTGGACCACACCTAGCCTGAAGTGC
ATCAGAGATCCCGCTCTGGTTCATCAGAGGCCTGCCCCTCCATCTACAGTGACAACAGCTGGCG
TGACCCCTCAGCCTGAGTCTCTGTCTCCATCTGGAAAAGAGCCTGCCGCCAGCTCTCCCAGCTC
TAACAATACTGCTGCCACCACAGCCGCTATCGTGCCTGGATCTCAGCTGATGCCTAGCAAGAGC
CCTAGCACCGGCACAACAGAGATCAGCTCTCACGAGAGCAGCCACGGAACACCTTCTCAGACCA
CCGCCAAGAATTGGGAGCTGACAGCCTCTGCCTCTCATCAGCCACCTGGCGTGTACCCACAGGG
CCACTCTGATACAACAGTGGCCATCAGCACCAGCACCGTTCTGCTGTGTGGCCTGTCTGCTGTT
AGCCTGCTGGCCTGCTACCTGAAGTCTAGACAGACACCTCCTCTGGCCAGCGTGGAAATGGAAG
CCATGGAAGCTCTGCCTGTCACATGGGGCACCAGCAGCAGAGATGAGGACCTCGAGAATTGCAG
CCACCACCTGTAAGCGGCCGCGTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGATTTGGCTACAGCAACAGGGTGGTGGACCTC
ATGGCCCACATGGCCTCCAAGGAGTAAGACCCCTGGACCACCAGCCCCAGCAAGAGCACAAGAG
GAAGAGAGAGACCCTCACTGCTGGGGAGTCCCTGCCACACTCAGTCCCCCACCACACTGAATCT
CCCCTCCTCACAGTTGCCATGTAGACCCCTTGAAGAGGGGAGGGGCCTAGGGAGCCGCACCTTG
TCATGTACCATCAATAAAGTACCCTGTGCTCAACCAGTTACTTGTCCTGTCTTATTCTAGGGTC
TGGGGCAGAGGGGAGGGAAGCTGGGCTTGTGTCAAGGTGAGACATTCTTGCTGGGGAGGGACCT
GGTATGTTCTCCTCAGACTGAGGGTAGGGCCTCCAAACAGCCTTGCTTGCTTCGAGAACCATTT
GCTTCCCGCTCAGACGTCTTGAGTGCTACAGGAAGCTGGCACCACTACTTCAGAGAACAAGGCC
TTTTCCTCTCCTCGCTCCAGT
exemplary donor template for insertion at GAPDH locus
SEQ ID NO: 214
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGTCAAATATTACAGATC
CACAGATGTGGGATTTTGATGATCTAAATTTCACTGGCATGCCACCTGCAGATGAAGATTACAG
CCCCTGTATGCTAGAAACTGAGACACTCAACAAGTATGTTGTGATCATCGCCTATGCCCTAGTG
TTCCTGCTGAGCCTGCTGGGAAACTCCCTGGTGATGCTGGTCATCTTATACAGCAGGGTCGGCC
GCTCCGTCACTGATGTCTACCTGCTGAACCTGGCCTTGGCCGACCTACTCTTTGCCCTGACCTT
GCCCATCTGGGCCGCCTCCAAGGTGAATGGCTGGATTTTTGGCACATTCCTGTGCAAGGTGGTC
TCACTCCTGAAGGAAGTCAACTTCTACAGTGGCATCCTGCTGTTGGCCTGCATCAGTGTGGACC
GTTACCTGGCCATTGTCCATGCCACACGCACACTGACCCAGAAGCGTCACTTGGTCAAGTTTGT
TTGTCTTGGCTGCTGGGGACTGTCTATGAATCTGTCCCTGCCCTTCTTCCTTTTCCGCCAGGCT
TACCATCCAAACAATTCCAGTCCAGTTTGCTATGAGGTCCTGGGAAATGACACAGCAAAATGGC
GGATGGTGTTGCGGATCCTGCCTCACACCTTTGGCTTCATCGTGCCGCTGTTTGTCATGCTGTT
CTGCTATGGATTCACCCTGCGTACACTGTTTAAGGCCCACATGGGGCAGAAGCACCGAGCCATG
AGGGTCATCTTTGCTGTCGTCCTCATCTTCCTGCTTTGCTGGCTGCCCTACAACCTGGTCCTGC
TGGCAGACACCCTCATGAGGACCCAGGTGATCCAGGAGAGCTGTGAGCGCCGCAACAACATCGG
CCGGGCCCTGGATGCCACTGAGATTCTGGGATTTCTCCATAGCTGCCTCAACCCCATCATCTAC
GCCTTCATCGGCCAAAATTTTCGCCATGGATTCCTCAAGATCCTGGCTATGCATGGCCTGGTCA
GCAAGGAGTTCTTGGCACGTCATCGTGTTACCTCCTACACTTCTTCGTCTGTCAATGTCTCTTC
CAACCTCTGAATTTGGCTACAGCAACAGGGTGGTGGACCTCATGGCCCACATGGCCTCCAAGGA
GTAAGACCCCTGGACCACCAGCCCCAGCAAGAGCACAAGAGGAAGAGAGAGACCCTCACTGCTG
GGGAGTCCCTGCCACACTCAGTCCCCCACCACACTGAATCTCCCCTCCTCACAGTTGCCATGTA
GACCCCTTGAAGAGGGGAGGGGCCTAGGGAGCCGCACCTTGTCATGTACCATCAATAAAGTACC
CTGTGCTCAACCAGTTACTTGTCCTGTCTTATTCTAGGGTCTGGGGCAGAGGGGAGGGAAGCTG
GGCTTGTGTCAAGGTGAGACATTCTTGCTGGGGAGGGACCTGGTATGTTCTCCTCAGACTGAGG
GTAGGGCCTCCAAACAGCCTTGCTTGCTTCGAGAACCATTTGCTTCCCGCTCAGACGTCTTGAG
TGCTACAGGAAGCTGGCACCACTACTTCAGAGAACAAGGCCTTTTCCTCTCCTCGCTCCAGT
exemplary donor template for insertion at GAPDH locus
SEQ ID NO: 215
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGAGTTGAGGAAGTACG
GCCCTGGAAGACTGGCGGGGACAGTTATAGGAGGAGCTGCTCAGAGTAAATCACAGACTAAATC
AGACTCAATCACAAAAGAGTTCCTGCCAGGCCTTTACACAGCCCCTTCCTCCCCGTTCCCGCCC
TCACAGGTGAGTGACCACCAAGTGCTAAATGACGCCGAGGTTGCCGCCCTCCTGGAGAACTTCA
GCTCTTCCTATGACTATGGAGAAAACGAGAGTGACTCGTGCTGTACCTCCCCGCCCTGCCCACA
GGACTTCAGCCTGAACTTCGACCGGGCCTTCCTGCCAGCCCTCTACAGCCTCCTCTTTCTGCTG
GGGCTGCTGGGCAACGGCGCGGTGGCAGCCGTGCTGCTGAGCCGGCGGACAGCCCTGAGCAGCA
CCGACACCTTCCTGCTCCACCTAGCTGTAGCAGACACGCTGCTGGTGCTGACACTGCCGCTCTG
GGCAGTGGACGCTGCCGTCCAGTGGGTCTTTGGCTCTGGCCTCTGCAAAGTGGCAGGTGCCCTC
TTCAACATCAACTTCTACGCAGGAGCCCTCCTGCTGGCCTGCATCAGCTTTGACCGCTACCTGA
ACATAGTTCATGCCACCCAGCTCTACCGCCGGGGGCCCCCGGCCCGCGTGACCCTCACCTGCCT
GGCTGTCTGGGGGCTCTGCCTGCTTTTCGCCCTCCCAGACTTCATCTTCCTGTCGGCCCACCAC
GACGAGCGCCTCAACGCCACCCACTGCCAATACAACTTCCCACAGGTGGGCCGCACGGCTCTGC
GGGTGCTGCAGCTGGTGGCTGGCTTTCTGCTGCCCCTGCTGGTCATGGCCTACTGCTATGCCCA
CATCCTGGCCGTGCTGCTGGTTTCCAGGGGCCAGCGGCGCCTGCGGGCCATGCGGCTGGTGGTG
GTGGTCGTGGTGGCCTTTGCCCTCTGCTGGACCCCCTATCACCTGGTGGTGCTGGTGGACATCC
TCATGGACCTGGGCGCTTTGGCCCGCAACTGTGGCCGAGAAAGCAGGGTAGACGTGGCCAAGTC
GGTCACCTCAGGCCTGGGCTACATGCACTGCTGCCTCAACCCGCTGCTCTATGCCTTTGTAGGG
GTCAAGTTCCGGGAGCGGATGTGGATGCTGCTCTTGCGCCTGGGCTGCCCCAACCAGAGAGGGC
TCCAGAGGCAGCCATCGTCTTCCCGCCGGGATTCATCCTGGTCTGAGACCTCAGAGGCCTCCTA
CTCGGGCTTGTGAATTTGGCTACAGCAACAGGGTGGTGGACCTCATGGCCCACATGGCCTCCAA
GGAGTAAGACCCCTGGACCACCAGCCCCAGCAAGAGCACAAGAGGAAGAGAGAGACCCTCACTG
CTGGGGAGTCCCTGCCACACTCAGTCCCCCACCACACTGAATCTCCCCTCCTCACAGTTGCCAT
GTAGACCCCTTGAAGAGGGGAGGGGCCTAGGGAGCCGCACCTTGTCATGTACCATCAATAAAGT
ACCCTGTGCTCAACCAGTTACTTGTCCTGTCTTATTCTAGGGTCTGGGGCAGAGGGGAGGGAAG
CTGGGCTTGTGTCAAGGTGAGACATTCTTGCTGGGGAGGGACCTGGTATGTTCTCCTCAGACTG
AGGGTAGGGCCTCCAAACAGCCTTGCTTGCTTCGAGAACCATTTGCTTCCCGCTCAGACGTCTT
GAGTGCTACAGGAAGCTGGCACCACTACTTCAGAGAACAAGGCCTTTTCCTCTCCTCGCTCCAG
T
exemplary donor template for insertion at GAPDH locus
SEQ ID NO: 216
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGTCCTTGAGGTGAGTG
ACCACCAAGTGCTAAATGACGCCGAGGTTGCCGCCCTCCTGGAGAACTTCAGCTCTTCCTATGA
CTATGGAGAAAACGAGAGTGACTCGTGCTGTACCTCCCCGCCCTGCCCACAGGACTTCAGCCTG
AACTTCGACCGGGCCTTCCTGCCAGCCCTCTACAGCCTCCTCTTTCTGCTGGGGCTGCTGGGCA
ACGGCGCGGTGGCAGCCGTGCTGCTGAGCCGGCGGACAGCCCTGAGCAGCACCGACACCTTCCT
GCTCCACCTAGCTGTAGCAGACACGCTGCTGGTGCTGACACTGCCGCTCTGGGCAGTGGACGCT
GCCGTCCAGTGGGTCTTTGGCTCTGGCCTCTGCAAAGTGGCAGGTGCCCTCTTCAACATCAACT
TCTACGCAGGAGCCCTCCTGCTGGCCTGCATCAGCTTTGACCGCTACCTGAACATAGTTCATGC
CACCCAGCTCTACCGCCGGGGGCCCCCGGCCCGCGTGACCCTCACCTGCCTGGCTGTCTGGGGG
CTCTGCCTGCTTTTCGCCCTCCCAGACTTCATCTTCCTGTCGGCCCACCACGACGAGCGCCTCA
ACGCCACCCACTGCCAATACAACTTCCCACAGGTGGGCCGCACGGCTCTGCGGGTGCTGCAGCT
GGTGGCTGGCTTTCTGCTGCCCCTGCTGGTCATGGCCTACTGCTATGCCCACATCCTGGCCGTG
CTGCTGGTTTCCAGGGGCCAGCGGCGCCTGCGGGCCATGCGGCTGGTGGTGGTGGTCGTGGTGG
CCTTTGCCCTCTGCTGGACCCCCTATCACCTGGTGGTGCTGGTGGACATCCTCATGGACCTGGG
CGCTTTGGCCCGCAACTGTGGCCGAGAAAGCAGGGTAGACGTGGCCAAGTCGGTCACCTCAGGC
CTGGGCTACATGCACTGCTGCCTCAACCCGCTGCTCTATGCCTTTGTAGGGGTCAAGTTCCGGG
AGCGGATGTGGATGCTGCTCTTGCGCCTGGGCTGCCCCAACCAGAGAGGGCTCCAGAGGCAGCC
ATCGTCTTCCCGCCGGGATTCATCCTGGTCTGAGACCTCAGAGGCCTCCTACTCGGGCTTGTGA
ATTTGGCTACAGCAACAGGGTGGTGGACCTCATGGCCCACATGGCCTCCAAGGAGTAAGACCCC
TGGACCACCAGCCCCAGCAAGAGCACAAGAGGAAGAGAGAGACCCTCACTGCTGGGGAGTCCCT
GCCACACTCAGTCCCCCACCACACTGAATCTCCCCTCCTCACAGTTGCCATGTAGACCCCTTGA
AGAGGGGAGGGGCCTAGGGAGCCGCACCTTGTCATGTACCATCAATAAAGTACCCTGTGCTCAA
CCAGTTACTTGTCCTGTCTTATTCTAGGGTCTGGGGCAGAGGGGAGGGAAGCTGGGCTTGTGTC
AAGGTGAGACATTCTTGCTGGGGAGGGACCTGGTATGTTCTCCTCAGACTGAGGGTAGGGCCTC
CAAACAGCCTTGCTTGCTTCGAGAACCATTTGCTTCCCGCTCAGACGTCTTGAGTGCTACAGGA
AGCTGGCACCACTACTTCAGAGAACAAGGCCTTTTCCTCTCCTCGCTCCAGT
exemplary donor template for insertion at GAPDH locus
SEQ ID NO: 217
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGGATTATCAAGTGTCAA
GTCCAATCTATGACATCAATTATTATACATCGGAGCCCTGCCAAAAAATCAATGTGAAGCAAAT
CGCAGCCCGCCTCCTGCCTCCGCTCTACTCACTGGTGTTCATCTTTGGTTTTGTGGGCAACATG
CTGGTCATCCTCATCCTGATAAACTGCAAAAGGCTGAAGAGCATGACTGACATCTACCTGCTCA
ACCTGGCCATCTCTGACCTGTTTTTCCTTCTTACTGTCCCCTTCTGGGCTCACTATGCTGCCGC
CCAGTGGGACTTTGGAAATACAATGTGTCAACTCTTGACAGGGCTCTATTTTATAGGCTTCTTC
TCTGGAATCTTCTTCATCATCCTCCTGACAATCGATAGGTACCTGGCTGTCGTCCATGCTGTGT
TTGCTTTAAAAGCCAGGACGGTCACCTTTGGGGTGGTGACAAGTGTGATCACTTGGGTGGTGGC
TGTGTTTGCGTCTCTCCCAGGAATCATCTTTACCAGATCTCAAAAAGAAGGTCTTCATTACACC
TGCAGCTCTCATTTTCCATACAGTCAGTATCAATTCTGGAAGAATTTCCAGACATTAAAGATAG
TCATCTTGGGGCTGGTCCTGCCGCTGCTTGTCATGGTCATCTGCTACTCGGGAATCCTAAAAAC
TCTGCTTCGGTGTCGAAATGAGAAGAAGAGGCACAGGGCTGTGAGGCTTATCTTCACCATCATG
ATTGTTTATTTTCTCTTCTGGGCTCCCTACAACATTGTCCTTCTCCTGAACACCTTCCAGGAAT
TCTTTGGCCTGAATAATTGCAGTAGCTCTAACAGGTTGGACCAAGCTATGCAGGTGACAGAGAC
TCTTGGGATGACGCACTGCTGCATCAACCCCATCATCTATGCCTTTGTCGGGGAGAAGTTCAGA
AACTACCTCTTAGTCTTCTTCCAAAAGCACATTGCCAAACGCTTCTGCAAATGCTGTTCTATTT
TCCAGCAAGAGGCTCCCGAGCGAGCAAGCTCAGTTTACACCCGATCCACTGGGGAGCAGGAAAT
ATCTGTGGGCTTGTGAATTTGGCTACAGCAACAGGGTGGTGGACCTCATGGCCCACATGGCCTC
CAAGGAGTAAGACCCCTGGACCACCAGCCCCAGCAAGAGCACAAGAGGAAGAGAGAGACCCTCA
CTGCTGGGGAGTCCCTGCCACACTCAGTCCCCCACCACACTGAATCTCCCCTCCTCACAGTTGC
CATGTAGACCCCTTGAAGAGGGGAGGGGCCTAGGGAGCCGCACCTTGTCATGTACCATCAATAA
AGTACCCTGTGCTCAACCAGTTACTTGTCCTGTCTTATTCTAGGGTCTGGGGCAGAGGGGAGGG
AAGCTGGGCTTGTGTCAAGGTGAGACATTCTTGCTGGGGAGGGACCTGGTATGTTCTCCTCAGA
CTGAGGGTAGGGCCTCCAAACAGCCTTGCTTGCTTCGAGAACCATTTGCTTCCCGCTCAGACGT
CTTGAGTGCTACAGGAAGCTGGCACCACTACTTCAGAGAACAAGGCCTTTTCCTCTCCTCGCTC
CAGT
exemplary donor template for insertion at GAPDH locus
SEQ ID NO: 218
GAAGACTGTGGATGGCCCCTCCGGGAAACTGTGGCGTGATGGCCGCGGGGCTCTCCAGAACATC
ATCCCTGCCTCTACTGGCGCTGCCAAGGCTGTGGGCAAGGTCATCCCTGAGCTGAACGGGAAGC
TCACTGGCATGGCCTTCCGTGTCCCCACTGCCAACGTGTCAGTGGTGGACCTGACCTGCCGTCT
AGAAAAACCTGCCAAATATGATGACATCAAGAAGGTGGTGAAGCAGGCGTCGGAGGGCCCCCTC
AAGGGCATCCTGGGCTACACTGAGCACCAGGTGGTCTCCTCTGACTTCAACAGCGACACCCACT
CCTCCACCTTTGACGCTGGGGCTGGCATTGCCCTCAACGACCACTTTGTCAAGCTCATTTCCTG
GTATGTGGCTGGGGCCAGAGACTGGCTCTTAAAAAGTGCAGGGTCTGGCGCCCTCTGGTGGCTG
GCTCAGAAAAAGGGCCCTGACAACTCTTTACATCTTCTAGGTATGACAACGAGTTCGGATATAG
CAATAGAGTGGTCGATCTGATGGCTCATATGGCTAGCAAAGAGGGAAGCGGAGCTACTAACTTC
AGCCTGCTGAAGCAGGCTGGAGACGTGGAGGAGAACCCTGGACCTATGCTGTCCACATCTCGTT
CTCGGTTTATCAGAAATACCAACGAGAGCGGTGAAGAAGTCACCACCTTTTTTGATTATGATTA
CGGTGCTCCCTGTCATAAATTTGACGTGAAGCAAATTGGGGCCCAACTCCTGCCTCCGCTCTAC
TCGCTGGTGTTCATCTTTGGTTTTGTGGGCAACATGCTGGTCGTCCTCATCTTAATAAACTGCA
AAAAGCTGAAGTGCTTGACTGACATTTACCTGCTCAACCTGGCCATCTCTGATCTGCTTTTTCT
TATTACTCTCCCATTGTGGGCTCACTCTGCTGCAAATGAGTGGGTCTTTGGGAATGCAATGTGC
AAATTATTCACAGGGCTGTATCACATCGGTTATTTTGGCGGAATCTTCTTCATCATCCTCCTGA
CAATCGATAGATACCTGGCTATTGTCCATGCTGTGTTTGCTTTAAAAGCCAGGACGGTCACCTT
TGGGGTGGTGACAAGTGTGATCACCTGGTTGGTGGCTGTGTTTGCTTCTGTCCCAGGAATCATC
TTTACTAAATGCCAGAAAGAAGATTCTGTTTATGTCTGTGGCCCTTATTTTCCACGAGGATGGA
ATAATTTCCACACAATAATGAGGAACATTTTGGGGCTGGTCCTGCCGCTGCTCATCATGGTCAT
CTGCTACTCGGGAATCCTGAAAACCCTGCTTCGGTGTCGAAACGAGAAGAAGAGGCATAGGGCA
GTGAGAGTCATCTTCACCATCATGATTGTTTACTTTCTCTTCTGGACTCCCTATAATATTGTCA
TTCTCCTGAACACCTTCCAGGAATTCTTCGGCCTGAGTAACTGTGAAAGCACCAGTCAACTGGA
CCAAGCCACGCAGGTGACAGAGACTCTTGGGATGACTCACTGCTGCATCAATCCCATCATCTAT
GCCTTCGTTGGGGAGAAGTTCAGAAGCCTTTTTCACATAGCTCTTGGCTGTAGGATTGCCCCAC
TCCAAAAACCAGTGTGTGGAGGTCCAGGAGTGAGACCAGGAAAGAATGTGAAAGTGACTACACA
AGGACTCCTCGATGGTCGTGGAAAAGGAAAGTCAATTGGCAGAGCCCCTGAAGCCAGTCTTCAG
GACAAAGAAGGAGCCTAGATTTGGCTACAGCAACAGGGTGGTGGACCTCATGGCCCACATGGCC
TCCAAGGAGTAAGACCCCTGGACCACCAGCCCCAGCAAGAGCACAAGAGGAAGAGAGAGACCCT
CACTGCTGGGGAGTCCCTGCCACACTCAGTCCCCCACCACACTGAATCTCCCCTCCTCACAGTT
GCCATGTAGACCCCTTGAAGAGGGGAGGGGCCTAGGGAGCCGCACCTTGTCATGTACCATCAAT
AAAGTACCCTGTGCTCAACCAGTTACTTGTCCTGTCTTATTCTAGGGTCTGGGGCAGAGGGGAG
GGAAGCTGGGCTTGTGTCAAGGTGAGACATTCTTGCTGGGGAGGGACCTGGTATGTTCTCCTCA
GACTGAGGGTAGGGCCTCCAAACAGCCTTGCTTGCTTCGAGAACCATTIGCTTCCCGCTCAGAC
GTCTTGAGTGCTACAGGAAGCTGGCACCACTACTTCAGAGAACAAGGCCTTTTCCTCTCCTCGC
TCCAGT

### Methods of Editing the Genome of a Cell for Gain-of-Function Modifications

In one aspect, the present disclosure provides methods of editing the genome of a cell. In certain embodiments, the method comprises contacting the cell with a nuclease that causes a break within an endogenous coding sequence of an essential gene in the cell wherein the essential gene encodes at least one gene product that is required for survival and/or proliferation of the cell. The cell is also contacted with (i) a donor template that comprises a knock-in cassette comprising an exogenous coding sequence for a gene product of interest in frame with and downstream (3′) of an exogenous coding sequence or partial coding sequence of the essential gene and/or (ii) a donor template that comprises a knock-in cassette comprising an exogenous coding sequence for a gene product of interest in frame with and upstream (5′) of an exogenous coding sequence or partial coding sequence of the essential gene (FIG. 3D). The knock-in cassette is integrated into the genome of the cell by homology-directed repair (HDR) of the break, resulting in a genome-edited cell that expresses the gene product of interest and the gene product encoded by the essential gene that is required for survival and/or proliferation of the cell, or a functional variant thereof. The genetically modified “knock-in” cell survives and proliferates to produce progeny cells with genomes that also include the exogenous coding sequence for the gene product of interest. This is illustrated in FIG. 3A for an exemplary method.

If the knock-in cassette is not properly integrated into the genome of the cell, undesired editing events that result from the break, e.g., NHEJ-mediated creation of indels, may produce a non-functional, e.g., out of frame, version of the essential gene. This produces a “knock-out” cell when the editing efficiency of the nuclease is high enough to disrupt both alleles. In certain embodiments, this produces a “knock-out” cell when the editing efficiency of the nuclease is high enough to disrupt one allele. Without sufficient functional copies of the essential gene these “knock-out” cells are unable to survive and do not produce any progeny cells.

In some embodiments, the present disclosure provides methods of editing the genome of a cell. In certain embodiments, the method comprises contacting the cell with a nuclease that causes a break within an endogenous non-coding sequence of an essential gene in the cell wherein the essential gene encodes at least one gene product that is required for survival and/or proliferation of the cell. In some embodiments, such a break within an endogenous non-coding sequence alters a functional region of an essential gene that influences post-transcriptional modification patterns, e.g., mRNA splicing, RNA stability, RNA editing, RNA interference, etc. In some embodiments, such a break within an endogenous non-coding sequence occurs in a functional region of the essential gene, for example, but not limited to: a splicesome target site (e.g., a 5′ splice donor site, an intron branch point sequence, a 3′ splice acceptor site, and/or a polypyrimidine tract), an intronic splicing silencer, an intronic splicing enhancer, an exonic splicing silencer, an exonic splicing enhancer, an endogenous RNA interference binding site (e.g., micro RNA, small interfering RNA, etc.), an endogenous RNA editing machinery binding site (e.g., a binding site for adenosine deaminases, cytidine deaminases, etc.), or combinations thereof. In some embodiments, the nuclease causes a break at or near where an intron borders an exon in an essential gene, reducing or disrupting the function of the essential gene.

Since the “knock-in” cells survive and the “knock-out” cells do not survive, the method automatically selects for the “knock-in” cells when it is applied to a population of starting cells. Significantly, in certain embodiments, the method does not require high knock-in efficiencies because of this automatic selection aspect. It is therefore particularly suitable for methods where the donor template is a dsDNA (e.g., a plasmid) where knock-in efficiencies are often below 5%. As noted in the exemplary method of FIG. 3C, in some embodiments some of the cells in the population of starting cells may remain unedited, i.e., unaffected by the nuclease. These cells would also survive and produce progeny with genomes that do not include the exogenous coding sequence for the gene product of interest. When the nuclease editing efficiency is high, e.g., about 60-90%, or higher the percentage of unedited cells will be relatively low as compared to the percentage of genetically modified cells. In some embodiments, high nuclease editing efficiencies (e.g., greater than 65%, greater than 70%, greater than 75%, greater than 80%, greater than 85%, greater than 90%, or greater than 95%) facilitates efficient population wide transgene integration, as the percentage of unedited cells will be relatively low as compared to the percentage of genetically modified cells. In some embodiments of the methods disclosed herein, at least about 65% of the cells (e.g., about 70%, about 75%, about 80%, about 85%, about 90%, about 91%, about 92%, about 93%, about 94%, about 95%, about 96%, about 97%, about 98%, or about 99% of the cells) are edited by a nuclease, e.g., a Cas12a, Cas9, Cas12b, Cas12c, Cas12e, CasX, or CasΦ (Cas12j), or a variant thereof (e.g., a variant with a high editing efficiency). In some embodiments, an RNP containing a CRISPR nuclease (e.g., Cas12a, Cas9, Cas12b, Cas12c, Cas12e, CasX, or CasΦ (Cas12j), or a variant thereof (e.g., a variant with a high editing efficiency)) and a guide are capable of cleaving the locus of an essential gene (e.g., a terminal exon in the locus of any essential gene provided in Table 3) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells). In some embodiments, an RNP containing a CRISPR nuclease (e.g., Cas12a, Cas9, Cas12b, Cas12c, Cas12e, CasX, or CasΦ (Cas12j), or a variant thereof (e.g., a variant with a high editing efficiency)) and a guide are capable of inducing knock-in cassette integration at a locus of an essential gene (e.g., a terminal exon in the locus of any essential gene provided in Table 3) in at least 65% of the cells in a population of cells (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells), e.g., at between 4 days and 10 days (e.g., 4 days, 5 days, 6 days, 7 days, 8 days, 9 days or 10 days) after the cells in the population of cells is contacted with the RNP containing a CRISPR nuclease. In some embodiments, editing efficiency is determined prior to target cell die off, e.g., at day 1 and/or day 2 post transfection or transduction. In some embodiments, editing efficiency measured at day 1 and/or day 2 post transfection or transduction may not capture the complete proportion of cells for which editing occurred, as in some embodiments, certain editing events may result in near immediate and/or swift cell death. In some embodiments, near immediate and/or swift cell death may be any period of time less than 48 hours post transfection or transduction, for example, less than 48 hours, less than 44 hours, less than 40 hours, less than 36 hours, less than 32 hours, less than 28 hours, less than 24 hours, less than 20 hours, less than 16 hours, less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, or less than 1 hour after transfection or transduction.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strands of a double-stranded DNA, e.g., genomic DNA of the cell.

In some embodiments, the present disclosure provides methods suitable for high-efficiency knock-in (e.g., a high proportion of a cell population comprises a knock-in allele), overcoming a major manufacturing challenge. In some embodiments, high-efficiency knock-in results in at least 65% of the cells in a population of cells comprising a knock-in allele (e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the cells in a population of cells comprise a knock-in allele). Historically, gene of interest knock-in using plasmid vectors results in efficiencies typically between 0.1 and 5% (see e.g., Zhu et al., CRISPR/Cas-Mediated Selection-free Knockin Strategy in Human Embryonic Stem Cells. Stem Cell Reports. 2015; 4(6):1103-1111), this low knock-in efficiency can result in a need for extensive time and resources devoted to screening potentially edited clones.

In some embodiments, a gene of interest knocked into a cell may have a role in effector function, specificity, stealth, persistence, homing/chemotaxis, and/or resistance to certain chemicals (see for example, Saetersmoen et al., Seminars in Immunopathology, 2019).

In certain embodiments, the present disclosure provides methods for creation of knock-in cells that maintain high levels of expression regardless of age, differentiation status, and/or exogenous conditions. For example, in some embodiments, an integrated cargo is expressed at an optimal level with a desired subcellular localization as a function of an insertion site. In some embodiments, the present disclosure provides such cells.

### Systems for Editing the Genome of a Cell

In one aspect the present disclosure provides systems for editing the genome of a cell. In some embodiments, the system comprises the cell, a nuclease that causes a break within an endogenous coding sequence of an essential gene of the cell, wherein the essential gene encodes a gene product that is required for survival and/or proliferation of the cell, and a donor template that comprises a knock-in cassette comprising an exogenous coding sequence for a gene product of interest in frame with and downstream (3′) of an exogenous coding sequence or partial coding sequence of the essential gene.

In some embodiments, the nuclease causes a double-strand break. In some embodiments the nuclease causes a single-strand break, e.g., in some embodiments the nuclease is a nickase. In some embodiments the nuclease is a prime editor which comprises a nickase domain fused to a reverse transcriptase domain. In some embodiments the nuclease is an RNA-guided prime editor and the gRNA comprises the donor template. In some embodiments a dual-nickase system is used which causes a double-strand break via two single-strand breaks on opposing strand of a double-stranded DNA, e.g., genomic DNA of the cell.

In one aspect, genome editing systems of the present disclosure may be used, for example, to edit stem cells. In some embodiments, genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (“Makarova”)), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”) describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”) describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (“Davis”) (describing Alt-HDR); Frit et al. DNA Repair 17(2014) 81-97 (“Frit”) (describing Alt-NHEJ); and Iyama and Wilson III, DNA Repair (Amst.) 2013 Aug. 12(8): 620-636 (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a target gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”). Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

### Nuclease

Any nuclease that causes a break within an endogenous genomic sequence, e.g., a coding sequence of an essential gene of the cell can be used in the methods of the present disclosure. In some embodiments the nuclease is a DNA nuclease. In some embodiments the nuclease causes a single-strand break (SSB) within an endogenous coding sequence of an essential gene of the cell, e.g., in a “prime editing” system. In some embodiments the nuclease causes a double-strand break (DSB) within an endogenous coding sequence of an essential gene of the cell. In some embodiments the double-strand break is caused by a single nuclease. In some embodiments the double-strand break is caused by two nucleases that each cause a single-strand break on opposing strands, e.g., a dual “nickase” system. In some embodiments the nuclease is a CRISPR/Cas nuclease and the method further comprises contacting the cell with one or more guide molecules for the CRISPR/Cas nuclease. Exemplary CRISPR/Cas nucleases and guide molecules are described in more detail herein. It is to be understood that the nuclease (including a nickase) is not limited in any manner and can also be a zinc finger nuclease (ZFN), a transcription activator-like effector nuclease (TALEN), a meganuclease, or other nuclease known in the art (or a combination thereof). Methods for designing zinc finger nucleases (ZFNs) are well known in the art, e.g., see Urnov et al., Nature Reviews Genetics 2010; 11:636-640 and Paschon et al., Nat. Commun. 2019; 10(1): 1133 and references cited therein. Methods for designing transcription activator-like effector nucleases (TALENs) are well known in the art, e.g., see Joung and Sander, Nat. Rev. Mol. Cell Biol. 2013; 14(1):49-55 and references cited therein. Methods for designing meganucleases are also well known in the art, e.g., see Silva et al., Curr. Gene Ther. 2011; 11(1): 11-27 and Redel and Prather, Toxicol. Pathol. 2016; 44(3):428-433.

In some embodiments, a nuclease suitable for methods described herein can have an editing efficiency that is greater than about 50%. In some embodiments, a nuclease suitable for methods described herein can have an editing efficiency that is greater than about 55%. In some embodiments, a nuclease suitable for methods described herein can have an editing efficiency that is greater than about 60%. In some embodiments, a nuclease suitable for methods described herein can have an editing efficiency that is greater than about 65%. In some embodiments, a nuclease suitable for methods described herein can have an editing efficiency that is greater than about 70%. In some embodiments, a nuclease suitable for methods described herein can have an editing efficiency that is greater than about 75%. In some embodiments, a nuclease suitable for methods described herein can have an editing efficiency that is greater than about 80%. In some embodiments, a nuclease suitable for methods described herein can have an editing efficiency that is greater than about 85%. In some embodiments, a nuclease suitable for methods described herein can have an editing efficiency that is greater than about 90%. In some embodiments, a nuclease suitable for methods described herein can have an editing efficiency that is greater than about 95%. In some embodiments, a nuclease suitable for methods described herein can have an editing efficiency that is greater than about 96%. In some embodiments, a nuclease suitable for methods described herein can have an editing efficiency that is greater than about 97%. In some embodiments, a nuclease suitable for methods described herein can have an editing efficiency that is greater than about 98%. In some embodiments, a nuclease suitable for methods described herein can have an editing efficiency that is greater than about 99%.

In general, the nuclease can be delivered to the cell as a protein or a nucleic acid encoding the protein, e.g., a DNA molecule or mRNA molecule. The protein or nucleic acid can be combined with other delivery agents, e.g., lipids or polymers in a lipid or polymer nanoparticle and targeting agents such as antibodies or other binding agents with specificity for the cell. The DNA molecule can be a nucleic acid vector, such as a viral genome or circular double-stranded DNA, e.g., a plasmid. Nucleic acid vectors encoding a nuclease can include other coding or non-coding elements. For example, a nuclease can be delivered as part of a viral genome (e.g., in an AAV, adenoviral or lentiviral genome) that includes certain genomic backbone elements (e.g., inverted terminal repeats, in the case of an AAV genome).

A CRISPR/Cas nuclease can be delivered to the cell as a protein or a nucleic acid encoding the protein, e.g., a DNA molecule or mRNA molecule. The guide molecule can be delivered as an RNA molecule or encoded by a DNA molecule. A CRISPR/Cas nuclease can also be delivered with a guide molecule as a ribonucleoprotein (RNP) and introduced into the cell via nucleofection (electroporation).

### CRISPR Cas Nucleases

CRISPR/Cas nucleases according to the present disclosure include, but are not limited to, naturally-occurring Class 2 CRISPR nucleases such as Cas9, and Cpf1 (Cas12a), as well as other Cas12 nucleases and nucleases derived or obtained therefrom. In functional terms, CRISPR/Cas nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, CRISPR/Cas nucleases can be defined, in broad terms, by their PAM specificity and cleavage activity, even though variations may exist between individual CRISPR/Cas nucleases that share the same PAM specificity or cleavage activity. Skilled artisans will appreciate that some aspects of the present disclosure relate to systems and methods that can be implemented using any suitable CRISPR/Cas nuclease having a certain PAM specificity and/or cleavage activity. For this reason, unless otherwise specified, the term CRISPR/Cas nuclease should be understood as a generic term, and not limited to any particular type (e.g., Cas9 vs. Cpf1), species (e.g., S. pyogenes vs. S, aureus) or variation (e.g., full-length vs. truncated or split; naturally-occurring PAM specificity vs. engineered PAM specificity, etc.) of CRISPR/Cas nuclease.

The PAM sequence takes its name from its sequential relationship to the “protospacer” sequence that is complementary to gRNA targeting domains (or “spacers”). Together with protospacer sequences, PAM sequences define target regions or sequences for specific CRISPR/Cas nuclease and gRNA combinations.

Various CRISPR/Cas nucleases may require different sequential relationships between PAMs and protospacers. In general, Cas9s recognize PAM sequences that are 3′ of the protospacer. Cpf1 (Cas12a), on the other hand, generally recognizes PAM sequences that are 5′ of the protospacer.

In addition to recognizing specific sequential orientations of PAMs and protospacers, CRISPR/Cas nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. F. novicida Cpf1 recognizes a TTN PAM sequence. PAM sequences have been identified for a variety of CRISPR/Cas nucleases, and a strategy for identifying novel PAM sequences has been described by Shmakov et al., Molecular Cell 2015; 60:385-397. It should also be noted that engineered CRISPR/Cas nucleases can have PAM specificities that differ from the PAM specificities of reference molecules (for instance, in the case of an engineered CRISPR/Cas nuclease, the reference molecule may be the naturally occurring variant from which the CRISPR/Cas nuclease is derived, or the naturally occurring variant having the greatest amino acid sequence homology to the engineered CRISPR/Cas nuclease).

In addition to their PAM specificity, CRISPR/Cas nucleases can be characterized by their DNA cleavage activity: naturally-occurring CRISPR/Cas nucleases typically form double-strand breaks (DSBs) in target nucleic acids, but engineered variants called “nickases” have been produced that generate only single-strand breaks (SSBs), e.g., those discussed in Ran et al., Cell 2013; 154(6):1380-1389 (“Ran”), or that that do not cut at all.

### Cas9

Crystal structures have been determined for S. pyogenes Cas9 (Jinek et al., Science 2014; 343(6176): 1247997 (“Jinek 2014”), and for S, aureus Cas9 in complex with a unimolecular guide RNA and a target DNA. See Nishimasu et al., Cell 1024; 156:935-949 (“Nishimasu 2014”); Nishimasu et al., Cell 2015; 162:1113-1126 (“Nishimasu 2015”); and Anders et al., Nature 2014; 513(7519):569-73 (“Anders 2014”).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g., a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e., bottom) strand of the target nucleic acid. It may be formed from two or more split RuvC motifs (such as RuvC I, RuvCII, and RuvCIII in S. pyogenes and S, aureus). The HNH domain, meanwhile, is structurally similar to HNN endonuclease motifs, and cleaves the complementary (i.e., top) strand of the target nucleic acid. The PI domain, as its name suggests, contributes to PAM specificity.

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

### Cpf1

The crystal structure of Acidaminococcus sp. Cpf1 in complex with crRNA and a dsDNA target including a TTTN PAM sequence has been solved by Yamano et al., Cell. 2016; 165(4): 949-962 (“Yamano”). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

### Nuclease Variants

The CRISPR/Cas nucleases described herein have activities and properties that can be useful in a variety of applications, but the skilled artisan will appreciate that CRISPR/Cas nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran, Yamano and PCT Publication No. WO 2016/073990A1, the entire contents of each of which are incorporated herein by reference. In general, mutations that reduce or eliminate activity in one of the two nuclease domains result in CRISPR/Cas nucleases with nickase activity, but it should be noted that the type of nickase activity varies depending on which domain is inactivated. As one example, inactivation of a RuvC domain or of a Cas9 HNH domain results in a nickase. Exemplary nickase variants include Cas9 D10A and Cas9 H840A (numbering scheme according to SpCas9 wild-type sequence). Additional suitable nickase variants, including Cas12a variants, will be apparent to the skilled artisan based on the present disclosure and the knowledge in the art. The present disclosure is not limited in this respect. In some embodiments a nickase may be fused to a reverse transcriptase to produce a prime editor (PE), e.g., as described in Anzalone et al., Nature 2019; 576:149-157, the entire contents of which are incorporated herein by reference.

Modifications of PAM specificity relative to naturally occurring Cas9 reference molecules has been described for both S. pyogenes (Kleinstiver et al., Nature 2015; 523(7561):481-5); and S. aureus (Kleinstiver et al., Nat Biotechnol. 2015; 33(12):1293-1298). Modifications that improve the targeting fidelity of Cas9 have also been described (Kleinstiver et al., Nature 2016; 529:490-495). Each of these references is incorporated by reference herein.

CRISPR/Cas nucleases have also been split into two or more parts, as described by Zetsche et al., Nat Biotechnol. 2015; 33(2): 139-42, incorporated by reference, and by Fine et al., Sci Rep. 2015; 5:10777, incorporated by reference.

CRISPR/Cas nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotech. 2014; 32:577-582, which is incorporated by reference herein.

CRISPR/Cas nucleases also optionally include a tag, such as, but not limited to, a nuclear localization signal, to facilitate movement of CRISPR/Cas nuclease protein into the nucleus. In certain embodiments, the CRISPR/Cas nuclease can incorporate C- and/or N-terminal nuclear localization signals. Nuclear localization sequences are known in the art.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular CRISPR/Cas nucleases, but it should be understood that the CRISPR/Cas nucleases used may be modified in ways that do not alter their operating principles. Such modifications are within the scope of the present disclosure.

Exemplary suitable nuclease variants include, but are not limited to, AsCpf1 (AsCas12a) variants comprising an M537R substitution, an H800A substitution, and/or an F870L substitution, or any combination thereof (numbering scheme according to AsCpf1 wild-type sequence). In some embodiments, a nuclease variant is a Cas12a variant, e.g., a Cas12a variant comprising 1, 2, or 3 of the amino acid substitutions selected from M537R, F870L, and H800A. In some embodiments, a Cas12a variant comprises an amino acid sequence having at least about 90%, 95%, or 100% identity to an AsCpf1 sequence described herein.

Other suitable modifications of the AsCpf1 amino acid sequence are known to those of ordinary skill in the art. Some exemplary sequences of wild-type AsCpf1 and AsCpf1 variants are provided below:

His-AsCpf1-sNLS-sNLS H800A amino acid sequence
SEQ ID NO: 58
MGHHHHHHGSTQFEGFTNLYQVSKTLRFELIPQGKTLKHIQEQGF
IEEDKARNDHYKELKPIIDRIYKTYADQCLQLVQLDWENLSAAID
SYRKEKTEETRNALIEEQATYRNAIHDYFIGRTDNLTDAINKRHA
EIYKGLFKAELFNGKVLKQLGTVTTTEHENALLRSFDKFTTYFSG
FYENRKNVESAEDISTAIPHRIVQDNFPKFKENCHIFTRLITAVP
SLREHFENVKKAIGIFVSTSIEEVFSFPFYNQLLTQTQIDLYNQL
LGGISREAGTEKIKGLNEVLNLAIQKNDETAHIIASLPHRFIPLF
KQILSDRNTLSFILEEFKSDEEVIQSFCKYKTLLRNENVLETAEA
LFNELNSIDLTHIFISHKKLETISSALCDHWDTLRNALYERRISE
LTGKITKSAKEKVQRSLKHEDINLQEIISAAGKELSEAFKQKTSE
ILSHAHAALDQPLPTTLKKQEEKEILKSQLDSLLGLYHLLDWFAV
DESNEVDPEFSARLTGIKLEMEPSLSFYNKARNYATKKPYSVEKF
KLNFQMPTLASGWDVNKEKNNGAILFVKNGLYYLGIMPKQKGRYK
ALSFEPTEKTSEGEDKMYYDYFPDAAKMIPKCSTQLKAVTAHFQT
HTTPILLSNNFIEPLEITKEIYDLNNPEKEPKKFQTAYAKKTGDQ
KGYREALCKWIDFTRDFLSKYTKTTSIDLSSLRPSSQYKDLGEYY
AELNPLLYHISFQRIAEKEIMDAVETGKLYLFQIYNKDFAKGHHG
KPNLHTLYWTGLFSPENLAKTSIKLNGQAELFYRPKSRMKRMAAR
LGEKMLNKKLKDQKTPIPDTLYQELYDYVNHRLSHDLSDEARALL
PNVITKEVSHEIIKDRRFTSDKFFFHVPITLNYQAANSPSKFNQR
VNAYLKEHPETPIIGIDRGERNLIYITVIDSTGKILEQRSLNTIQ
QFDYQKKLDNREKERVAARQAWSVVGTIKDLKQGYLSQVIHEIVD
LMIHYQAVVVLENLNFGFKSKRTGIAEKAVYQQFEKMLIDKLNCL
VLKDYPAEKVGGVLNPYQLTDQFTSFAKMGTQSGFLFYVPAPYTS
KIDPLTGFVDPFVWKTIKNHESRKHFLEGFDFLHYDVKTGDFILH
FKMNRNLSFQRGLPGEMPAWDIVFEKNETQFDAKGTPFIAGKRIV
PVIENHRFTGRYRDLYPANELIALLEEKGIVERDGSNILPKLLEN
DDSHAIDTMVALIRSVLQMRNSNAATGEDYINSPVRDLNGVCFDS
RFQNPEWPMDADANGAYHIALKGQLLLNHLKESKDLKLQNGISNQ
DWLAYIQELRNGSPKKKRKVGSPKKKRKV
Cpf1 variant 1 amino acid sequence
SEQ ID NO: 59
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVESFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGEMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 2 amino acid sequence
SEQ ID NO: 60
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVE
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLESPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 3 amino acid sequence
SEQ ID NO: 61
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVE
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDELSKYTKTTSIDLSSLRPSSQYKDLGEYYAELNPLLYH
ISFQRIAEKEIMDAVETGKLYLFQIYNKDFAKGHHGKPNLHTLYW
TGLESPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKELFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGEMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 4 amino acid sequence
SEQ ID NO: 62
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVESFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAARLGEKMLNKK
LKDQKTPIPDTLYQELYDYVNHRLSHDLSDEARALLPNVITKEVS
HEIIKDRRFTSDKFLFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLIGFV
DPFVWKTIKNHESRKHFLEGFDFLHYDVKTGDFILHFKMNRNLSF
QRGLPGEMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 5 amino acid sequence
SEQ ID NO: 63
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVE
SAEDISTAIPHRIVQDNFPKFKENCHIFTRLITAVPSLREHFENV
KKAIGIFVSTSIEEVESFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDELSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFLFHVPITLNYQAANSPSKFNQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGFMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKV
Cpf1 variant 6 amino acid sequence
SEQ ID NO: 64
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVF
SAEDISTAIPHRIVQDNFPKFKENCHIFTRLITAVPSLREHFENV
KKAIGIFVSTSIEEVFSFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKILLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQRPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDFLSKYTKTTSIDLSSLRPSSQYKDLGEYYAELNPLLYH
ISFQRIAEKEIMDAVETGKLYLFQIYNKDFAKGHHGKPNLHTLYW
TGLESPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKELFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRTGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGEMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RNGRSSDDEATADSQHAAPPKKKRKVGGSGGSGGSGGSGGSGGSG
GSGGSLEHHHHHH
Cpf1 variant 7 amino acid sequence
SEQ ID NO: 65
MGRDPGKPIPNPLLGLDSTAPKKKRKVGIHGVPAATQFEGFTNLY
QVSKTLRFELIPQGKTLKHIQEQGFIEEDKARNDHYKELKPIIDR
IYKTYADQCLQLVQLDWENLSAAIDSYRKEKTEETRNALIEEQAT
YRNAIHDYFIGRTDNLTDAINKRHAEIYKGLFKAELFNGKVLKQL
GTVTTTEHENALLRSFDKFTTYFSGFYENRKNVESAEDISTAIPH
RIVQDNFPKFKENCHIFTRLITAVPSLREHFENVKKAIGIFVSTS
IEEVFSFPFYNQLLTQTQIDLYNQLLGGISREAGTEKIKGLNEVL
NLAIQKNDETAHIIASLPHRFIPLFKQILSDRNTLSFILEEFKSD
EEVIQSFCKYKTLLRNENVLETAEALFNELNSIDLTHIFISHKKL
ETISSALCDHWDTLRNALYERRISELTGKITKSAKEKVQRSLKHE
DINLQEIISAAGKELSEAFKQKTSEILSHAHAALDQPLPTTLKKQ
EEKEILKSQLDSLLGLYHLLDWFAVDESNEVDPEFSARLTGIKLE
MEPSLSFYNKARNYATKKPYSVEKFKLNFQMPTLASGWDVNKEKN
NGAILFVKNGLYYLGIMPKQKGRYKALSFEPTEKTSEGFDKMYYD
YFPDAAKMIPKCSTQLKAVTAHFQTHTTPILLSNNFIEPLEITKE
IYDLNNPEKEPKKFQTAYAKKTGDQKGYREALCKWIDFTRDFLSK
YTKTTSIDLSSLRPSSQYKDLGEYYAELNPLLYHISFQRIAEKEI
MDAVETGKLYLFQIYNKDFAKGHHGKPNLHTLYWTGLESPENLAK
TSIKLNGQAELFYRPKSRMKRMAHRLGEKMLNKKLKDQKTPIPDT
LYQELYDYVNHRLSHDLSDEARALLPNVITKEVSHEIIKDRRFTS
DKFFFHVPITLNYQAANSPSKFNQRVNAYLKEHPETPIIGIDRGE
RNLIYITVIDSTGKILEQRSLNTIQQFDYQKKLDNREKERVAARQ
AWSVVGTIKDLKQGYLSQVIHEIVDLMIHYQAVVVLENLNFGFKS
KRTGIAEKAVYQQFEKMLIDKLNCLVLKDYPAEKVGGVLNPYQLT
DQFTSFAKMGTQSGFLFYVPAPYTSKIDPLTGFVDPFVWKTIKNH
ESRKHFLEGFDFLHYDVKTGDFILHFKMNRNLSFQRGLPGFMPAW
DIVFEKNETQFDAKGTPFIAGKRIVPVIENHRFTGRYRDLYPANE
LIALLEEKGIVERDGSNILPKLLENDDSHAIDTMVALIRSVLQMR
NSNAATGEDYINSPVRDLNGVCFDSRFQNPEWPMDADANGAYHIA
LKGQLLLNHLKESKDLKLQNGISNQDWLAYIQELRNPKKKRKVKL
AAALEHHHHHH
Exemplary AsCpf1 wild-type amino acid sequence
SEQ ID NQ: 66
MTQFEGFTNLYQVSKTLRFELIPQGKTLKHIQEQGFIEEDKARND
HYKELKPIIDRIYKTYADQCLQLVQLDWENLSAAIDSYRKEKTEE
TRNALIEEQATYRNAIHDYFIGRTDNLTDAINKRHAEIYKGLFKA
ELFNGKVLKQLGTVTTTEHENALLRSFDKFTTYFSGFYENRKNVE
SAEDISTAIPHRIVQDNFPKFKENCHIFTRLITAVPSLREHFENV
KKAIGIFVSTSIEEVESFPFYNQLLTQTQIDLYNQLLGGISREAG
TEKIKGLNEVLNLAIQKNDETAHIIASLPHRFIPLFKQILSDRNT
LSFILEEFKSDEEVIQSFCKYKTLLRNENVLETAEALFNELNSID
LTHIFISHKKLETISSALCDHWDTLRNALYERRISELTGKITKSA
KEKVQRSLKHEDINLQEIISAAGKELSEAFKQKTSEILSHAHAAL
DQPLPTTLKKQEEKEILKSQLDSLLGLYHLLDWFAVDESNEVDPE
FSARLTGIKLEMEPSLSFYNKARNYATKKPYSVEKFKLNFQMPTL
ASGWDVNKEKNNGAILFVKNGLYYLGIMPKQKGRYKALSFEPTEK
TSEGFDKMYYDYFPDAAKMIPKCSTQLKAVTAHFQTHTTPILLSN
NFIEPLEITKEIYDLNNPEKEPKKFQTAYAKKTGDQKGYREALCK
WIDFTRDELSKYTKTTSIDLSSLRPSSQYKDLGEYYAELNPLLYH
ISFQRIAEKEIMDAVETGKLYLFQIYNKDFAKGHHGKPNLHTLYW
TGLFSPENLAKTSIKLNGQAELFYRPKSRMKRMAHRLGEKMLNKK
LKDQKTPIPDTLYQELYDYVNHRLSHDLSDEARALLPNVITKEVS
HEIIKDRRFTSDKFFFHVPITLNYQAANSPSKENQRVNAYLKEHP
ETPIIGIDRGERNLIYITVIDSTGKILEQRSLNTIQQFDYQKKLD
NREKERVAARQAWSVVGTIKDLKQGYLSQVIHEIVDLMIHYQAVV
VLENLNFGFKSKRIGIAEKAVYQQFEKMLIDKLNCLVLKDYPAEK
VGGVLNPYQLTDQFTSFAKMGTQSGFLFYVPAPYTSKIDPLTGFV
DPFVWKTIKNHESRKHFLEGFDFLHYDVKTGDFILHFKMNRNLSF
QRGLPGEMPAWDIVFEKNETQFDAKGTPFIAGKRIVPVIENHRFT
GRYRDLYPANELIALLEEKGIVERDGSNILPKLLENDDSHAIDTM
VALIRSVLQMRNSNAATGEDYINSPVRDLNGVCFDSRFQNPEWPM
DADANGAYHIALKGQLLLNHLKESKDLKLQNGISNQDWLAYIQEL
RN

Additional suitable nucleases and nuclease variants will be apparent to the skilled artisan based on the present disclosure in view of the knowledge in the art. Exemplary suitable nucleases may include but are not limited to those provided in Table 5

TABLE 5
Exemplary Suitable CRISPR/Cas Nucleases

| | Length | | |
|---|---|---|---|
| Nuclease | (A.A.) | PAM | Reference |
| SpCas9 | 1368 | NGG | Cong et al., Science 2013; |
| | | | 339(6121): 819-23 |
| SaCas9 | 1053 | NNGRRT | Ran et al., Nature 2015; |
| | | | 520(7546): 186-91. |
| (KKH) | 1067 | NNNRRT | Kleinstiver et al., |
| SaCas9 | | | Nat Biotechnol. 2015; |
| | | | 33(12): 1293-1298 |
| AsCpf1 | 1353 | TTTV | Zetsche et al., Nat Biotechnol. |
| (AsCas12a) | | | 2017; 35(1): 31-34 |
| LbCpf1 | 1274 | TTTV | Zetsche et al., Cell 2015; |
| (LbCas12a) | | | 163(3): 759-71. |
| CasX | 980 | TTC | Burstein et al., Nature 2017; |
| | | | 542(7640): 237-241. |
| CasY | 1200 | TA | Burstein et al., Nature 2017; |
| | | | 542(7640): 237-241. |
| Cas12h1 | 870 | RTR | Yan et al., Science 2019; |
| | | | 363(6422): 88-91. |
| Cas12i1 | 1093 | TTN | Yan et al., Science 2019; |
| | | | 363(6422): 88-91. |
| Cas12c1 | unknown | TG | Yan et al., Science 2019; |
| | | | 363(6422): 88-91. |
| Cas12c2 | unknown | TN | Yan et al., Science 2019; |
| | | | 363(6422): 88-91. |
| eSpCas9 | 1423 | NGG | Chen et al., Nature 2017; |
| | | | 550(7676): 407-410. |
| Cas9-HF1 | 1367 | NGG | Chen et al., Nature 2017; |
| | | | 550(7676): 407-410. |
| HypaCas9 | 1404 | NGG | Chen et al., Nature 2017; |
| | | | 550(7676): 407-410. |
| dCas9-Fokl | 1623 | NGG | U.S. Pat. No. 9,322,037 |
| Sniper-Cas9 | 1389 | NGG | Lee et al., Nat Commun. |
| | | | 2018; 9(1): 3048. |
| xCas9 | 1786 | NGG, NG, | Hu et al., Nature. 2018 |
| | | GAA, GAT | Apr. 5; 556(7699): 57-63. |
| AaCas12b | 1129 | TTN | Teng et al., Cell Discov. |
| | | | 2018; 4: 63. |
| evoCas9 | 1423 | NGG | Casini et al., Nat Biotechnol. |
| | | | 2018; 36(3): 265-271. |
| SpCas9-NG | 1423 | NG | Nishimasu et al., Science |
| | | | 2018; 361(6408): 1259-1262. |
| VRQR | 1368 | NGA | Li et al., The CRISPR |
| | | | Journal, 2018; 01: 01 |
| VRER | 1372 | NGCG | Kleinstiver et al., Nature |
| | | | 2016; 529(7587): 490-5. |
| NmeCas9 | 1082 | NNNNGATT | Amrani et al., Genome Biol. |
| | | | 2018; 19(1): 214. |
| CjCas9 | 984 | NNNNRYAC | Kim et al., Nat Commun. |
| | | | 2017; 8: 14500. |
| BhCas12b | 1108 | ATTN | Strecker et al., Nat Commun. |
| | | | 2019; 10(1): 212. |
| BhCas12b V4 | 1108 | ATTN | Pausch et al., Science 2020; |
| | | | 369(6501): 333-337. |
| CasΦ | 700-800 | TBN (where | Pausch et al., Science 2020; |
| | | B is G, T, or | 369(6501): 333-337. |
| | | C) | |

Guide RNA (gRNA) Molecules

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al., Molecular Cell 2014; 56(2): 333-339 (“Briner”), and in PCT Publication No. WO2016/073990A1.

In bacteria and archaea, type II CRISPR systems generally comprise an CRISPR/Cas nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). See Mali et al., Science 2013; 339(6121):823-826 (“Mali”); Jiang et al., Nat Biotechnol. 2013; 31(3):233-239 (“Jiang”); and Jinek et al., Science 2012; 337(6096):816-821 (“Jinek 2012”).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013; 31(9):827-832, (“Hsu”)), “complementarity regions” (PCT Publication No. WO2016/073990A1), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. See Nishimasu 2014 and 2015. It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. See Nishimasu 2015. A first stem-loop one near the 3′ portion of the second complementarity domain is referred to variously as the “proximal domain,” (PCT Publication No. WO2016/073990A1) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other CRISPR/Cas nucleases have been (or may in the future be) discovered or invented which utilize gRNAs that differ in some ways from those described to this point. For instance, Cpf1 (“CRISPR from Prevotella and Franciscella 1”) which is also called Cas12a is a CRISPR/Cas nuclease that does not require a tracrRNA to function (see Zetsche et al., Cell 2015; 163:759-771 (“Zetsche I”)). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple CRISPR/Cas nucleases. For this reason, unless otherwise specified, the term gRNA should be understood to encompass any suitable gRNA that can be used with any CRISPR/Cas nuclease, and not only those gRNAs that are compatible with a particular species of Cas9 or Cpf1. By way of illustration, the term gRNA can, in certain embodiments, include a gRNA for use with any CRISPR/Cas nuclease occurring in a Class 2 CRISPR system, such as a type II or type V or CRISPR system, or an CRISPR/Cas nuclease derived or adapted therefrom.

In some embodiments a method or system of the present disclosure may use more than one gRNA. In some embodiments, two or more gRNAs may be used to create two or more double strand breaks in the genome of a cell. In some embodiments, a multiplexed editing strategy may be used that targets two or more essential genes at the same time with two or more knock-in cassettes. In some such embodiments, the two or more knock-in cassettes may comprise different exogenous cargo sequences, e.g., different knock-in cassettes may encode different gene products of interest and thus the edited cells will express a plurality of gene products of interest from different knock-in cassettes targeted to different loci.

In some embodiments using more than one gRNA, a double-strand break may be caused by a dual-gRNA paired “nickase” strategy. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs.

In some embodiments, a method or system of the present disclosure may use a prime editing gRNA (pegRNA) in conjunction with a prime editor (PE). As is well known in the art, a pegRNA is substantially larger than standard gRNAs, e.g., in some embodiments longer than 50, 100, 150 or 250 nucleotides, e.g., as described in Anzalone et al., Nature 2019; 576:149-157, the entire contents of which are incorporated herein by reference. The pegRNA is a gRNA with a primer binding sequence (PBS) and a donor template containing the desired RNA sequence added at one of the termini, e.g., the 3′ end. The PE:pegRNA complex binds to the target DNA, and the nickase domain of the prime editor nicks only one strand, generating a flap. The PBS, located on the pegRNA, binds to the DNA flap and the edited RNA sequence is reverse transcribed using the reverse transcriptase domain of the prime editor. The edited strand is incorporated into the DNA at the end of the nicked flap, and the target DNA is repaired with the new reverse transcribed DNA. The original DNA segment is removed by a cellular endonuclease. This leaves one strand edited, and one strand unedited. In the newest PE systems, e.g., PE3 and PE3b, the unedited strand can be corrected to match the newly edited strand by using an additional standard gRNA. In this case, the unedited strand is nicked by a nickase and the newly edited strand is used as a template to repair the nick, thus completing the edit.

gRNA Design

Methods for selection and validation of target sequences as well as off-target analyses have been described previously, e.g., in Mali; Hsu; Fu et al., Nat Biotechnol 2014; 32(3):279-84, Heigwer et al., Nat methods 2014; 11(2):122-3; Bae et al., Bioinformatics 2014; 30(10): 1473-5; and Xiao et al. Bioinformatics 2014; 30(8):1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in PCT Publication No. WO2016/073990A1.

For example, methods for selection and validation of target sequences as well as off-target analyses can be performed using cas-offinder (Bae et al., Bioinformatics 2014; 30:1473-5). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench et al., Nat Biotechnol. 2016; 34:184-91.

gRNA Modifications

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases.

Exemplary suitable 5′ extensions for Cpf1 guide RNAs are provided in Table 6 below:

TABLE 6
Exemplary Cpf1 gRNA 5′ Extensions

| | | 5′ |
|---|---|---|
| SEQ ID NO: | 5′ extension sequence | modification |
| N/A | rCrUrUrUrU | +5 RNA |
| 67 | rArArGrArCrCrUrUrUrU | +10 RNA |
| 68 | rArUrGrUrGrUrUrUrUrUrGrUrCrArArArArGrArCr | +25 RNA |
| | CrUrUrUrU | |
| 69 | rArGrGrCrCrArGrCrUrUrGrCrCrGrGrUrUrUrUrUr | +60 RNA |
| | UrArGrUrCrGrUrGrCrUrGrCrUrUrCrArUrGrUrGr | |
| | UrUrUrUrUrGrUrCrArArArArGrArCrCrUrUrUrU | |
| N/A | CTTTT | +5 DNA |
| 70 | AAGACCTTTT | +10 DNA |
| 71 | ATGTGTTTTTGTCAAAAGACCTTTT | +25 DNA |
| 72 | AGGCCAGCTTGCCGGTTTTTTAGTCGTGCTGC | +60 DNA |
| | TTCATGTGTTTTTGTCAAAAGACCTTTT | |
| 73 | TTTTTGTCAAAAGACCTTTT | +20 DNA |
| 74 | GCTTCATGTGTTTTTGTCAAAAGACCTTTT | +30 DNA |
| 75 | GCCGGTTTTTTAGTCGTGCTGCTTCATGTGTT | +50 DNA |
| | TTTGTCAAAAGACCTTTT | |
| 76 | TAGTCGTGCTGCTTCATGTGTTTTTGTCAAAA | +40 DNA |
| | GACCTTTT | |
| 77 | C*C*GAAGTTTTCTTCGGTTTT | +20 DNA + |
| | | 2xPS |
| 78 | T*T*TTTCCGAAGTTTTCTTCGGTTTT | +25 DNA + |
| | | 2xPS |
| 79 | A*A*CGCTTTTTCCGAAGTTTTCTTCGGTTTT | +30 DNA + |
| | | 2xPS |
| 80 | G*C*GTTGTTTTCAACGCTTTTTCCGAAGTTTT | +41 DNA + |
| | CTTCGGTTTT | 2xPS |
| 81 | G*G*CTTCTTTTGAAGCCTTTTTGCGTTGTTTT | +62 DNA + |
| | CAACGCTTTTTCCGAAGTTTTCTTCGGTTTT | 2xPS |
| 82 | A*T*GTGTTTTTGTCAAAAGACCTTTT | +25 DNA + |
| | | 2xPS |
| 83 | AAAAAAAAAAAAAAAAAAAAAAAAA | +25 A |
| 84 | TTTTTTTTTTTTTTTTTTTTTTTTT | +25 T |
| 85 | mA*mU*rGrUrGrUrUrUrUrUrGrUrCrArArArArGr | +25 RNA + |
| | ArCrCrUrUrUrU | 2xPS |
| 86 | mA*mA*rArArArArArArArArArArArArArArArAr | PolyA RNA |
| | ArArArArArArA | + 2xPS |
| 87 | mU*mU*rUrUrUrUrUrUrUrUrUrUrUrUrUrUrUrUr | PolyU RNA |
| | UrUrUrUrUrUrU | + 2xPS |

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth herein.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a CRISPR/Cas nuclease, e.g., an AsCpf1 nuclease, to form an RNP, which is then employed to edit a target cell, e.g., a pluripotent stem cell or a progeny thereof.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

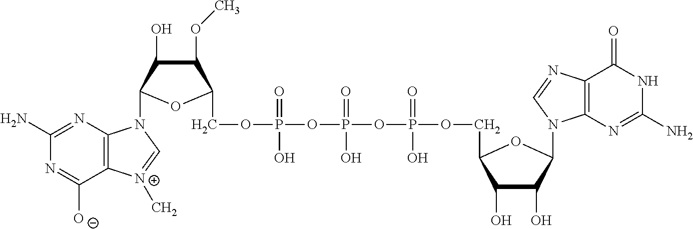

The cap or cap analog can be included during either chemical or enzymatic synthesis of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E. coli Poly(A)Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

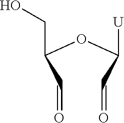

wherein “U” can be an unmodified or modified uridine.

The 3′ terminal U ribose can be modified with a 2′3′ cyclic phosphate as shown below:

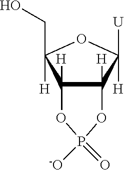

wherein “U” can be an unmodified or modified uridine.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂, alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, O- and N-alkylated nucleotides, e.g., N6-methyl adenosine, can be incorporated into a gRNA. In certain embodiments, one or more or all of the nucleotides in a gRNA are deoxynucleotides.

Guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl, carboxyl, hydroxyl, alkene (e.g., a terminal alkene), azide and/or another suitable functional group. Multifunctional (e.g. bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine, TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed. 2013, published by Academic Press.

Additional suitable gRNA modifications will be apparent to those of ordinary skill in the art based on the present disclosure. Suitable gRNA modifications include, for example, those described in PCT Publication No. WO2019070762A1 entitled “MODIFIED CPF1 GUIDE RNA;” in PCT Publication No. WO2016089433A1 entitled “GUIDE RNA WITH CHEMICAL MODIFICATIONS;” in PCT Publication No. WO2016164356A1 entitled “CHEMICALLY MODIFIED GUIDE RNAS FOR CRISPR/CAS-MEDIATED GENE REGULATION;” and in PCT Publication No. WO2017053729A1 entitled “NUCLEASE-MEDIATED GENOME EDITING OF PRIMARY CELLS AND ENRICHMENT THEREOF;” the entire contents of each of which are incorporated herein by reference.

Exemplary gRNAs

Non-limiting examples of guide RNAs suitable for certain embodiments embraced by the present disclosure are provided herein, for example, in the Tables below. Those of ordinary skill in the art will be able to envision suitable guide RNA sequences for a specific nuclease, e.g., a Cas9 or Cpf1 nuclease, from the disclosure of the targeting domain sequence, either as a DNA or RNA sequence. For example, a guide RNA comprising a targeting sequence consisting of RNA nucleotides would include the RNA sequence corresponding to the targeting domain sequence provided as a DNA sequence, and this contain uracil instead of thymidine nucleotides. For example, a guide RNA comprising a targeting domain sequence consisting of RNA nucleotides, and described by the DNA sequence TCTGCAGAAATGTTCCCCGT (SEQ ID NO: 88) would have a targeting domain of the corresponding RNA sequence UCUGCAGAAAUGUUCCCCGU (SEQ ID NO: 89). As will be apparent to the skilled artisan, such a targeting sequence would be linked to a suitable guide RNA scaffold, e.g., a crRNA scaffold sequence or a chimeric crRNA/tracrRNA scaffold sequence. Suitable gRNA scaffold sequences are known to those of ordinary skill in the art. For AsCpf1, for example, a suitable scaffold sequence comprises the sequence UAAUUUCUACUCUUGUAGAU (SEQ ID NO: 90), added to the 5′-terminus of the targeting domain. In the example above, this would result in a Cpf1 guide RNA of the sequence UAAUUUCUACUCUUGUAGAUUCUGCAGAAAUGUUCCCCGU (SEQ ID NO: 91). Those of skill in the art would further understand how to modify such a guide RNA, e.g., by adding a DNA extension (e.g., in the example above, adding a 25-mer DNA extension as described herein would result, for example, in a guide RNA of the sequence ATGTGTTTTTGTCAAAAGACCTTTTrUrArArUrUrUrCrUrArCrUrCrUrUrGrUrArGrArUrUr CrUrGrCrArGrArArArUrGrUrUrCrCrCrCrGrU (SEQ ID NO: 92)). It will be understood that the exemplary targeting sequences provided herein are not limiting, and additional suitable sequences, e.g., variants of the specific sequences disclosed herein, will be apparent to the skilled artisan based on the present disclosure in view of the general knowledge in the art.

In some embodiments the gRNA for use in the disclosure is a gRNA targeting TGFβRII (TGFβRII gRNA). In some embodiments, the gRNA targeting TGFβRII is one or more of the gRNAs described in Table 7.

TABLE 7
Exemplary TGFβRII gRNAs

| | gRNA Targeting | | | SEQ |
|---|---|---|---|---|
| | Domain | | | ID |
| Name | Sequence (DNA) | Length | Enzyme | NO: |
| TGFBR24326 | CAGGACGATGTGCAGCGGCC | 20 | AsCpf1 RR | 29 |
| TGFBR24327 | ACCGCACGTTCAGAAGTCGG | 20 | AsCpf1 RR | 30 |
| TGFBR24328 | ACAACTGTGTAAATTTTGTG | 20 | AsCpf1 RR | 31 |
| TGFBR24329 | CAACTGTGTAAATTTTGTGA | 20 | AsCpf1 RR | 32 |
| TGFBR24330 | ACCTGTGACAACCAGAAATC | 20 | AsCpf1 RR | 33 |
| TGFBR24331 | CCTGTGACAACCAGAAATCC | 20 | AsCpf1 RR | 34 |
| TGFBR24332 | TGTGGCTTCTCACAGATGGA | 20 | AsCpf1 RR | 35 |
| TGFBR24333 | TCTGTGAGAAGCCACAGGAA | 20 | AsCpf1 RR | 36 |
| TGFBR24334 | AAGCTCCCCTACCATGACTT | 20 | AsCpf1 RR | 37 |
| TGFBR24335 | GAATAAAGTCATGGTAGGGG | 20 | AsCpf1 RR | 38 |
| TGFBR24336 | AGAATAAAGTCATGGTAGGG | 20 | AsCpf1 RR | 39 |
| TGFBR24337 | CTACCATGACTTTATTCTGG | 20 | AsCpf1 RR | 40 |
| TGFBR24338 | TACCATGACTTTATTCTGGA | 20 | AsCpf1 RR | 41 |
| TGFBR24339 | TAATGCACTTTGGAGAAGCA | 20 | AsCpf1 RR | 42 |
| TGFBR24340 | TTCATAATGCACTTTGGAGA | 20 | AsCpf1 RR | 43 |
| TGFBR24341 | AAGTGCATTATGAAGGAAAA | 20 | AsCpf1 RR | 44 |
| TGFBR24342 | TGTGTTCCTGTAGCTCTGAT | 20 | AsCpf1 RR | 45 |
| TGFBR24343 | TGTAGCTCTGATGAGTGCAA | 20 | AsCpf1 RR | 46 |
| TGFBR24344 | AGTGACAGGCATCAGCCTCC | 20 | AsCpf1 RR | 47 |
| TGFBR24345 | AGTGGTGGCAGGAGGCTGAT | 20 | AsCpf1 RR | 48 |
| TGFBR24346 | AGGTTGAACTCAGCTTCTGC | 20 | AsCpf1 RR | 49 |
| TGFBR24347 | CAGGTTGAACTCAGCTTCTG | 20 | AsCpf1 RR | 50 |
| TGFBR24348 | ACCTGGGAAACCGGCAAGAC | 20 | AsCpf1 RR | 51 |
| TGFBR24349 | CGTCTTGCCGGTTTCCCAGG | 20 | AsCpf1 RR | 52 |
| TGFBR24350 | GCGTCTTGCCGGTTTCCCAG | 20 | AsCpf1 RR | 53 |
| TGFBR24351 | TGAGCTTCCGCGTCTTGCCG | 20 | AsCpf1 RR | 54 |
| TGFBR24352 | GCGAGCACTGTGCCATCATC | 20 | AsCpf1 RR | 55 |
| TGFBR24353 | GGATGATGGCACAGTGCTCG | 20 | AsCpf1 RR | 56 |
| TGFBR24354 | AGGATGATGGCACAGTGCTC | 20 | AsCpf1 RR | 57 |
| TGFBR24355 | CGTGTGCCAACAACATCAAC | 20 | AsCpf1 RR | 58 |
| TGFBR24356 | GCTCAATGGGCAGCAGCTCT | 20 | AsCpf1 RR | 59 |
| TGFBR24357 | ACCAGGGTGTCCAGCTCAAT | 20 | AsCpf1 RR | 60 |
| TGFBR24358 | CACCAGGGTGTCCAGCTCAA | 20 | AsCpf1 RR | 61 |
| TGFBR24359 | CCACCAGGGTGTCCAGCTCA | 20 | AsCpf1 RR | 62 |
| TGFBR24360 | GCTTGGCCTTATAGACCTCA | 20 | AsCpf1 RR | 63 |
| TGFBR24361 | GAGCAGTTTGAGACAGTGGC | 20 | AsCpf1 RR | 64 |
| TGFBR24362 | AGAGGCATACTCCTCATAGG | 20 | AsCpf1 RR | 65 |
| TGFBR24363 | CTATGAGGAGTATGCCTCTT | 20 | AsCpf1 RR | 66 |
| TGFBR24364 | AAGAGGCATACTCCTCATAG | 20 | AsCpf1 RR | 67 |
| TGFBR24365 | TATGAGGAGTATGCCTCTTG | 20 | AsCpf1 RR | 68 |
| TGFBR24366 | GATTGATGTCTGAGAAGATG | 20 | AsCpf1 RR | 69 |
| TGFBR24367 | CTCCTCAGCCGTCAGGAACT | 20 | AsCpf1 RR | 70 |
| TGFBR24368 | GTTCCTGACGGCTGAGGAGC | 20 | AsCpf1 RR | 71 |
| TGFBR24369 | GCTCCTCAGCCGTCAGGAAC | 20 | AsCpf1 RR | 72 |
| TGFBR24370 | TGACGGCTGAGGAGCGGAAG | 20 | AsCpf1 RR | 73 |
| TGFBR24371 | TCTTCCGCTCCTCAGCCGTC | 20 | AsCpf1 RR | 74 |
| TGFBR24372 | AACTCCGTCTTCCGCTCCTC | 20 | AsCpf1 RR | 75 |
| TGFBR24373 | CAACTCCGTCTTCCGCTCCT | 20 | AsCpf1 RR | 76 |
| TGFBR24374 | CCAACTCCGTCTTCCGCTCC | 20 | AsCpf1 RR | 77 |
| TGFBR24375 | ACGCCAAGGGCAACCTACAG | 20 | AsCpf1 RR | 78 |
| TGFBR24376 | CGCCAAGGGCAACCTACAGG | 20 | AsCpf1 RR | 79 |
| TGFBR24377 | AGCTGATGACATGCCGCGTC | 20 | AsCpf1 RR | 80 |
| TGFBR24378 | GGGCGAGGGAGCTGCCCAGC | 20 | AsCpf1 RR | 81 |
| TGFBR24379 | CGGGCGAGGGAGCTGCCCAG | 20 | AsCpf1 RR | 82 |
| TGFBR24380 | CCGGGCGAGGGAGCTGCCCA | 20 | AsCpf1 RR | 83 |
| TGFBR24381 | TCGCCCGGGGGATTGCTCAC | 20 | AsCpf1 RR | 84 |
| TGFBR24382 | ACATGGAGTGTGATCACTGT | 20 | AsCpf1 RR | 85 |
| TGFBR24383 | CAGTGATCACACTCCATGTG | 20 | AsCpf1 RR | 86 |
| TGFBR24384 | TGTGGGAGGCCCAAGATGCC | 20 | AsCpf1 RR | 87 |
| TGFBR24385 | TGTGCACGATGGGCATCTTG | 20 | AsCpf1 RR | 88 |
| TGFBR24386 | CGAGGATATTGGAGCTCTTG | 20 | AsCpf1 RR | 89 |
| TGFBR24387 | ATATCCTCGTGAAGAACGAC | 20 | AsCpf1 RR | 90 |
| TGFBR24388 | GACGCAGGGAAAGCCCAAAG | 20 | AsCpf1 RR | 91 |
| TGFBR24389 | CTGCGTCTGGACCCTACTCT | 20 | AsCpf1 RR | 92 |
| TGFBR24390 | TGCGTCTGGACCCTACTCTG | 20 | AsCpf1 RR | 93 |
| TGFBR24391 | CAGACAGAGTAGGGTCCAGA | 20 | AsCpf1 RR | 94 |
| TGFBR24392 | GCCAGCACGATCCCACCGCA | 20 | AsCpf1 RVR | 95 |
| TGFBR24393 | AAGGAAAAAAAAAAGCCTGG | 20 | AsCpf1 RVR | 96 |
| TGFBR24394 | ACACCAGCAATCCTGACTTG | 20 | AsCpf1 RVR | 97 |
| TGFBR24395 | ACTAGCAACAAGTCAGGATT | 20 | AsCpf1 RVR | 98 |
| TGFBR24396 | GCAACTCCCAGTGGTGGCAG | 20 | AsCpf1 RVR | 99 |
| TGFBR24397 | TGTCATCATCATCTTCTACT | 20 | AsCpf1 RVR | 100 |
| TGFBR24398 | GACCTCAGCAAAGCGACCTT | 20 | AsCpf1 RVR | 101 |
| TGFBR24399 | AGGCCAAGCTGAAGCAGAAC | 20 | AsCpf1 RVR | 102 |
| TGFBR24400 | AGGAGTATGCCTCTTGGAAG | 20 | AsCpf1 RVR | 103 |
| TGFBR24401 | CCTCTTGGAAGACAGAGAAG | 20 | AsCpf1 RVR | 104 |
| TGFBR24402 | TTCTCATGCTTCAGATTGAT | 20 | AsCpf1 RVR | 105 |
| TGFBR24403 | CTCGTGAAGAACGACCTAAC | 20 | AsCpf1 RVR | 106 |
| TGFbR2036 | GGCCGCTGCACATCGTCCTG | 20 | SpyCas9 | 107 |
| TGFbR2037 | GCGGGGTCTGCCATGGGTCG | 20 | SpyCas9 | 108 |
| TGFbR2038 | AGTTGCTCATGCAGGATTTC | 20 | SpyCas9 | 109 |
| TGFbR2039 | CCAGAATAAAGTCATGGTAG | 20 | SpyCas9 | 110 |
| TGFbR2040 | CCCCTACCATGACTTTATTC | 20 | SpyCas9 | 111 |
| TGFbR2041 | AAGTCATGGTAGGGGAGCTT | 20 | SpyCas9 | 112 |
| TGFbR2042 | AGTCATGGTAGGGGAGCTTG | 20 | SpyCas9 | 113 |
| TGFbR2043 | ATTGCACTCATCAGAGCTAC | 20 | SpyCas9 | 114 |
| TGFbR2044 | CCTAGAGTGAAGAGATTCAT | 20 | SpyCas9 | 115 |
| TGFbR2045 | CCAATGAATCTCTTCACTCT | 20 | SpyCas9 | 116 |
| TGFbR2046 | AAAGTCATGGTAGGGGAGCT | 20 | SpyCas9 | 117 |
| TGFbR2047 | GTGAGCAATCCCCCGGGCGA | 20 | SpyCas9 | 118 |
| TGFbR2048 | GTCGTTCTTCACGAGGATAT | 20 | SpyCas9 | 119 |
| TGFbR2049 | GCCGCGTCAGGTACTCCTGT | 20 | SpyCas9 | 120 |
| TGFbR2050 | GACGCGGCATGTCATCAGCT | 20 | SpyCas9 | 121 |
| TGFbR2051 | GCTTCTGCTGCCGGTTAACG | 20 | SpyCas9 | 122 |
| TGFbR2052 | GTGGATGACCTGGCTAACAG | 20 | SpyCas9 | 123 |
| TGFbR2053 | GTGATCACACTCCATGTGGG | 20 | SpyCas9 | 124 |
| TGFbR2054 | GCCCATTGAGCTGGACACCC | 20 | SpyCas9 | 125 |
| TGFbR2055 | GCGGTCATCTTCCAGGATGA | 20 | SpyCas9 | 126 |
| TGFbR2056 | GGGAGCTGCCCAGCTTGCGC | 20 | SpyCas9 | 127 |
| TGFbR2057 | GTTGATGTTGTTGGCACACG | 20 | SpyCas9 | 128 |
| TGFbR2058 | GGCATCTTGGGCCTCCCACA | 20 | SpyCas9 | 129 |
| TGFbR2059 | GCGGCATGTCATCAGCTGGG | 20 | SpyCas9 | 130 |
| TGFbR2060 | GCTCCTCAGCCGTCAGGAAC | 20 | SpyCas9 | 131 |
| TGFbR2061 | GCTGGTGTTATATTCTGATG | 20 | SpyCas9 | 132 |
| TGFbR2062 | CCGACTTCTGAACGTGCGGT | 20 | SpyCas9 | 133 |
| TGFbR2063 | TGCTGGCGATACGCGTCCAC | 20 | SpyCas9 | 134 |
| TGFbR2064 | CCCGACTTCTGAACGTGCGG | 20 | SpyCas9 | 135 |
| TGFbR2065 | CCACCGCACGTTCAGAAGTC | 20 | SpyCas9 | 136 |
| TGFbR2066 | TCACCCGACTTCTGAACGTG | 20 | SpyCas9 | 137 |
| TGFbR2067 | CCCACCGCACGTTCAGAAGT | 20 | SpyCas9 | 138 |
| TGFbR2068 | CGAGCAGCGGGGTCTGCCAT | 20 | SpyCas9 | 139 |
| TGFbR2069 | ACGAGCAGCGGGGTCTGCCA | 20 | SpyCas9 | 140 |
| TGFbR2070 | AGCGGGGTCTGCCATGGGTC | 20 | SpyCas9 | 141 |
| TGFbR2071 | CCTGAGCAGCCCCCGACCCA | 20 | SpyCas9 | 142 |
| TGFbR2072 | CCATGGGTCGGGGGCTGCTC | 20 | SpyCas9 | 143 |
| TGFbR2073 | AACGTGCGGTGGGATCGTGC | 20 | SpyCas9 | 144 |
| TGFbR2074 | GGACGATGTGCAGCGGCCAC | 20 | SpyCas9 | 145 |
| TGFbR2075 | GTCCACAGGACGATGTGCAG | 20 | SpyCas9 | 146 |
| TGFbR2076 | CATGGGTCGGGGGCTGCTCA | 20 | SpyCas9 | 147 |
| TGFbR2077 | CAGCGGGGTCTGCCATGGGT | 20 | SpyCas9 | 148 |
| TGFbR2078 | ATGGGTCGGGGGCTGCTCAG | 20 | SpyCas9 | 149 |
| TGFbR2079 | CGGGGTCTGCCATGGGTCGG | 20 | SpyCas9 | 150 |
| TGFbR2080 | AGGAAGTCTGTGTGGCTGTA | 20 | SpyCas9 | 151 |
| TGFbR2081 | CTCCATCTGTGAGAAGCCAC | 20 | SpyCas9 | 152 |
| TGFbR2082 | ATGATAGTCACTGACAACAA | 20 | SpyCas9 | 153 |
| TGFbR2083 | GATGCTGCAGTTGCTCATGC | 20 | SpyCas9 | 154 |
| TGFbR2084 | ACAGCCACACAGACTTCCTG | 20 | SpyCas9 | 155 |
| TGFbR2085 | GAAGCCACAGGAAGTCTGTG | 20 | SpyCas9 | 156 |
| TGFbR2086 | TTCCTGTGGCTTCTCACAGA | 20 | SpyCas9 | 157 |
| TGFbR2087 | CTGTGGCTTCTCACAGATGG | 20 | SpyCas9 | 158 |
| TGFbR2088 | TCACAAAATTTACACAGTTG | 20 | SpyCas9 | 159 |
| TGFbR2089 | GACAACATCATCTTCTCAGA | 20 | SpyCas9 | 160 |
| TGFbR2090 | TCCAGAATAAAGTCATGGTA | 20 | SpyCas9 | 161 |
| TGFbR2091 | GGTAGGGGAGCTTGGGGTCA | 20 | SpyCas9 | 162 |
| TGFbR2092 | TTCTCCAAAGTGCATTATGA | 20 | SpyCas9 | 163 |
| TGFbR2093 | CATCTTCCAGAATAAAGTCA | 20 | SpyCas9 | 164 |
| TGFbR2094 | CACATGAAGAAAGTCTCACC | 20 | SpyCas9 | 165 |
| TGFbR2095 | TTCCAGAATAAAGTCATGGT | 20 | SpyCas9 | 166 |
| TGFbR2096 | TTTTCCTTCATAATGCACTT | 20 | SpyCas9 | 167 |
| TGFBR24024 | CACAGTTGTGGAAACTTGAC | 20 | AsCpf1 | 168 |
| TGFBR24039 | CCCAACTCCGTCTTCCGCTC | 20 | AsCpf1 | 169 |
| TGFBR24040 | GGCTTTCCCTGCGTCTGGAC | 20 | AsCpf1 | 170 |
| TGFBR24036 | CTGAGGTCTATAAGGCCAAG | 20 | AsCpf1 | 171 |
| TGFBR24026 | TGATGTGAGATTTTCCACCT | 20 | AsCpf1 | 172 |
| TGFBR24038 | CCTATGAGGAGTATGCCTCT | 20 | AsCpf1 | 173 |
| TGFBR24033 | AAGTGACAGGCATCAGCCTC | 20 | AsCpf1 | 174 |
| TGFBR24028 | CCATGACCCCAAGCTCCCCT | 20 | AsCpf1 | 175 |
| TGFBR24031 | CTTCATAATGCACTTTGGAG | 20 | AsCpf1 | 176 |
| TGFBR24032 | TTCATGTGTTCCTGTAGCTC | 20 | AsCpf1 | 177 |
| TGFBR24029 | TTCTGGAAGATGCTGCTTCT | 20 | AsCpf1 | 178 |
| TGFBR24035 | CCCACCAGGGTGTCCAGCTC | 20 | AsCpf1 | 179 |
| TGFBR24037 | AGACAGTGGCAGTCAAGATC | 20 | AsCpf1 | 180 |
| TGFBR24041 | CCTGCGTCTGGACCCTACTC | 20 | AsCpf1 | 181 |
| TGFBR24025 | CACAACTGTGTAAATTTTGT | 20 | AsCpf1 | 182 |
| TGFBR24030 | GAGAAGCAGCATCTTCCAGA | 20 | AsCpf1 | 183 |
| TGFBR24027 | TGGTTGTCACAGGTGGAAAA | 20 | AsCpf1 | 184 |
| TGFBR24034 | CCAGGTTGAACTCAGCTTCT | 20 | AsCpf1 | 185 |
| TGFBR24043 | ATCACAAAATTTACACAGTTG | 21 | SauCas9 | 186 |
| TGFBR24065 | GGCATCAGCCTCCTGCCACCA | 21 | SauCas9 | 187 |
| TGFBR24110 | GTTAGCCAGGTCATCCACAGA | 21 | SauCas9 | 188 |
| TGFBR24099 | GCTGGGCAGCTCCCTCGCCCG | 21 | SauCas9 | 189 |
| TGFBR24064 | CAGGAGGCTGATGCCTGTCAC | 21 | SauCas9 | 190 |
| TGFBR24094 | GAGGAGCGGAAGACGGAGTTG | 21 | SauCas9 | 191 |
| TGFBR24108 | CGTCTGGACCCTACTCTGTCT | 21 | SauCas9 | 192 |
| TGFBR24058 | TTTTTCCTTCATAATGCACTT | 21 | SauCas9 | 193 |
| TGFBR24075 | CCATTGAGCTGGACACCCTGG | 21 | SauCas9 | 194 |
| TGFBR24057 | CTTCTCCAAAGTGCATTATGA | 21 | SauCas9 | 195 |
| TGFBR24103 | GCCCAAGATGCCCATCGTGCA | 21 | SauCas9 | 196 |
| TGFBR24060 | TCATGTGTTCCTGTAGCTCTG | 21 | SauCas9 | 197 |
| TGFBR24048 | GTGATGCTGCAGTTGCTCATG | 21 | SauCas9 | 198 |
| TGFBR24087 | TCTCATGCTTCAGATTGATGT | 21 | SauCas9 | 199 |
| TGFBR24081 | TCCCTATGAGGAGTATGCCTC | 21 | SauCas9 | 200 |
| TGFBR24044 | CATCACAAAATTTACACAGTT | 21 | SauCas9 | 201 |
| TGFBR24077 | ATTGAGCTGGACACCCTGGTG | 21 | SauCas9 | 202 |
| TGFBR24080 | CAGTCAAGATCTTTCCCTATG | 21 | SauCas9 | 203 |
| TGFBR24046 | AGGATTTCTGGTTGTCACAGG | 21 | SauCas9 | 204 |
| TGFBR24101 | TCCACAGTGATCACACTCCAT | 21 | SauCas9 | 205 |
| TGFBR24079 | AGCAGAACACTTCAGAGCAGT | 21 | SauCas9 | 206 |
| TGFBR24072 | CCGGCAAGACGCGGAAGCTCA | 21 | SauCas9 | 207 |
| TGFBR24074 | GATGTCAGAGCGGTCATCTTC | 21 | SauCas9 | 208 |
| TGFBR24062 | TCATTGCACTCATCAGAGCTA | 21 | SauCas9 | 209 |
| TGFBR24054 | CTTCCAGAATAAAGTCATGGT | 21 | SauCas9 | 210 |
| TGFBR24045 | AGATTTTCCACCTGTGACAAC | 21 | SauCas9 | 211 |
| TGFBR24049 | ACTGCAGCATCACCTCCATCT | 21 | SauCas9 | 212 |
| TGFBR24098 | AGCTGGGCAGCTCCCTCGCCC | 21 | SauCas9 | 213 |
| TGFBR24090 | TGACGGCTGAGGAGCGGAAGA | 21 | SauCas9 | 214 |
| TGFBR24076 | CATTGAGCTGGACACCCTGGT | 21 | SauCas9 | 215 |
| TGFBR24078 | AGCAAAGCGACCTTTCCCCAC | 21 | SauCas9 | 216 |
| TGFBR24067 | CGCGTTAACCGGCAGCAGAAG | 21 | SauCas9 | 217 |
| TGFBR24063 | GAAATATGACTAGCAACAAGT | 21 | SauCas9 | 218 |
| TGFBR24107 | AGACAGAGTAGGGTCCAGACG | 21 | SauCas9 | 219 |
| TGFBR24047 | CAGGATTTCTGGTTGTCACAG | 21 | SauCas9 | 220 |
| TGFBR24096 | CTCCTGTAGGTTGCCCTTGGC | 21 | SauCas9 | 221 |
| TGFBR24105 | ACAGAGTAGGGTCCAGACGCA | 21 | SauCas9 | 222 |
| TGFBR24056 | GCTTCTCCAAAGTGCATTATG | 21 | SauCas9 | 223 |
| TGFBR24068 | GCAGCAGAAGCTGAGTTCAAC | 21 | SauCas9 | 224 |
| TGFBR24093 | TGAGGAGCGGAAGACGGAGTT | 21 | SauCas9 | 225 |
| TGFBR24055 | CTTTGGAGAAGCAGCATCTTC | 21 | SauCas9 | 226 |
| TGFBR24053 | CTCCCCTACCATGACTTTATT | 21 | SauCas9 | 227 |
| TGFBR24106 | GACAGAGTAGGGTCCAGACGC | 21 | SauCas9 | 228 |
| TGFBR24092 | CTGAGGAGCGGAAGACGGAGT | 21 | SauCas9 | 229 |
| TGFBR24102 | GGGCATCTTGGGCCTCCCACA | 21 | SauCas9 | 230 |
| TGFBR24082 | CCAAGAGGCATACTCCTCATA | 21 | SauCas9 | 231 |
| TGFBR24051 | AGAATGACGAGAACATAACAC | 21 | SauCas9 | 232 |
| TGFBR24097 | CCTGACGCGGCATGTCATCAG | 21 | SauCas9 | 233 |
| TGFBR24073 | AGCGAGCACTGTGCCATCATC | 21 | SauCas9 | 234 |
| TGFBR24104 | GCAGGTTAGGTCGTTCTTCAC | 21 | SauCas9 | 235 |
| TGFBR24050 | ACCTCCATCTGTGAGAAGCCA | 21 | SauCas9 | 236 |
| TGFBR24052 | TAAAGTCATGGTAGGGGAGCT | 21 | SauCas9 | 237 |
| TGFBR24061 | TCAGAGCTACAGGAACACATG | 21 | SauCas9 | 238 |
| TGFBR24086 | TCTCAGACATCAATCTGAAGC | 21 | SauCas9 | 239 |
| TGFBR24066 | CATCAGCCTCCTGCCACCACT | 21 | SauCas9 | 240 |
| TGFBR24089 | CGCTCCTCAGCCGTCAGGAAC | 21 | SauCas9 | 241 |
| TGFBR24071 | AACCTGGGAAACCGGCAAGAC | 21 | SauCas9 | 242 |
| TGFBR24095 | TCCACGCCAAGGGCAACCTAC | 21 | SauCas9 | 243 |
| TGFBR24100 | GAGGTGAGCAATCCCCCGGGC | 21 | SauCas9 | 244 |
| TGFBR24069 | CAGCAGAAGCTGAGTTCAACC | 21 | SauCas9 | 245 |
| TGFBR24083 | TCCAAGAGGCATACTCCTCAT | 21 | SauCas9 | 246 |
| TGFBR24070 | AGCAGAAGCTGAGTTCAACCT | 21 | SauCas9 | 247 |
| TGFBR24088 | CCAGTTCCTGACGGCTGAGGA | 21 | SauCas9 | 248 |
| TGFBR24085 | AGGAGTATGCCTCTTGGAAGA | 21 | SauCas9 | 249 |
| TGFBR24084 | TTCCAAGAGGCATACTCCTCA | 21 | SauCas9 | 250 |
| TGFBR24042 | CAACTGTGTAAATTTTGTGAT | 21 | SauCas9 | 251 |
| TGFBR24059 | TGAAGGAAAAAAAAAAGCCTG | 21 | SauCas9 | 252 |
| TGFBR24091 | CGTCTTCCGCTCCTCAGCCGT | 21 | SauCas9 | 253 |
| TGFBR24109 | CCAGGTCATCCACAGACAGAG | 21 | SauCas9 | 254 |
| TGFBR2736 | GCCTAGAGTGAAGAGATTCAT | 21 | SpyCas9 | 255 |
| TGFBR2737 | GTTCTCCAAAGTGCATTATGA | 21 | SpyCas9 | 256 |
| TGFBR2738 | GCATCTTCCAGAATAAAGTCA | 21 | SpyCas9 | 257 |
| TGFBR2739 | TGATGTGAGATTTTCCACCTG | 21 | Cas12a | 1172 |

In some embodiments the gRNA for use in the disclosure is a gRNA targeting CISH (CISH gRNA). In some embodiments, the gRNA targeting CISH is one or more of the gRNAs described in Table 8.

TABLE 8
Exemplary CISH gRNAs

| | gRNA Targeting Domain | | | SEQ ID |
|---|---|---|---|---|
| Name | Sequence (DNA) | Length | Enzyme | NO: |
| CISH0873 | CAACCGTCTGGTGGCCGACG | 20 | SpyCas9 | 258 |
| CISH0874 | CAGGATCGGGGCTGTCGCTT | 20 | SpyCas9 | 259 |
| CISH0875 | TCGGGCCTCGCTGGCCGTAA | 20 | SpyCas9 | 260 |
| CISH0876 | GAGGTAGTCGGCCATGCGCC | 20 | SpyCas9 | 261 |
| CISH0877 | CAGGTGTTGTCGGGCCTCGC | 20 | SpyCas9 | 262 |
| CISH0878 | GGAGGTAGTCGGCCATGCGC | 20 | SpyCas9 | 263 |
| CISH0879 | GGCATACTCAATGCGTACAT | 20 | SpyCas9 | 264 |
| CISH0880 | CCGCCTTGTCATCAACCGTC | 20 | SpyCas9 | 265 |
| CISH0881 | AGGATCGGGGCTGTCGCTTC | 20 | SpyCas9 | 266 |
| CISH0882 | CCTTGTCATCAACCGTCTGG | 20 | SpyCas9 | 267 |
| CISH0883 | TACTCAATGCGTACATTGGT | 20 | SpyCas9 | 268 |
| CISH0884 | GGGTTCCATTACGGCCAGCG | 20 | SpyCas9 | 269 |
| CISH0885 | GGCACTGCTTCTGCGTACAA | 20 | SpyCas9 | 270 |
| CISH0886 | GGTTGATGACAAGGCGGCAC | 20 | SpyCas9 | 271 |
| CISH0887 | TGCTGGGGCCTTCCTCGAGG | 20 | SpyCas9 | 272 |
| CISH0888 | TTGCTGGCTGTGGAGCGGAC | 20 | SpyCas9 | 273 |
| CISH0889 | TTCTCCTACCTTCGGGAATC | 20 | SpyCas9 | 274 |
| CISH0890 | GACTGGCTTGGGCAGTTCCA | 20 | SpyCas9 | 275 |
| CISH0891 | CATGCAGCCCTTGCCTGCTG | 20 | SpyCas9 | 276 |
| CISH0892 | AGCAAAGGACGAGGTCTAGA | 20 | SpyCas9 | 277 |
| CISH0893 | GCCTGCTGGGGCCTTCCTCG | 20 | SpyCas9 | 278 |
| CISH0894 | CAGACTCACCAGATTCCCGA | 20 | SpyCas9 | 279 |
| CISH0895 | ACCTCGTCCTTTGCTGGCTG | 20 | SpyCas9 | 280 |
| CISH0896 | CTCACCAGATTCCCGAAGGT | 20 | SpyCas9 | 281 |
| CISH7048 | TACGCAGAAGCAGTGCCCGC | 20 | AsCpf1 | 282 |
| CISH7049 | AGGTGTACAGCAGTGGCTGG | 20 | AsCpf1 | 283 |
| CISH7050 | GGTGTACAGCAGTGGCTGGT | 20 | AsCpf1 | 284 |
| CISH7051 | CGGATGTGGTCAGCCTTGTG | 20 | AsCpf1 | 285 |
| CISH7052 | CACTGACAGCGTGAACAGGT | 20 | AsCpf1 | 286 |
| CISH7053 | ACTGACAGCGTGAACAGGTA | 20 | AsCpf1 | 287 |
| CISH7054 | GCTCACTCTCTGTCTGGGCT | 20 | AsCpf1 | 288 |
| CISH7055 | CTGGCTGTGGAGCGGACTGG | 20 | AsCpf1 | 289 |
| CISH7056 | GCTCTGACTGTACGGGGCAA | 20 | AsCpf1 RR | 290 |
| CISH7057 | AGCTCTGACTGTACGGGGCA | 20 | AsCpf1 RR | 291 |
| CISH7058 | ACAGTACCCCTTCCAGCTCT | 20 | AsCpf1 RR | 292 |
| CISH7059 | CGTCGGCCACCAGACGGTTG | 20 | AsCpf1 RR | 293 |
| CISH7060 | CCAGCCACTGCTGTACACCT | 20 | AsCpf1 RR | 294 |
| CISH7061 | ACCCCGGCCCTGCCTATGCC | 20 | AsCpf1 RR | 295 |
| CISH7062 | GGTATCAGCAGTGCAGGAGG | 20 | AsCpf1 RR | 296 |
| CISH7063 | GATGTGGTCAGCCTTGTGCA | 20 | AsCpf1 RR | 297 |
| CISH7064 | GGATGTGGTCAGCCTTGTGC | 20 | AsCpf1 RR | 298 |
| CISH7065 | GGCCACGCATCCTGGCCTTT | 20 | AsCpf1 RR | 299 |
| CISH7066 | GAAAGGCCAGGATGCGTGGC | 20 | AsCpf1 RR | 300 |
| CISH7067 | ACTGCTTGTCCAGGCCACGC | 20 | AsCpf1 RR | 301 |
| CISH7068 | TCTGGACTCCAACTGCTTGT | 20 | AsCpf1 RR | 302 |
| CISH7069 | GTCTGGACTCCAACTGCTTG | 20 | AsCpf1 RR | 303 |
| CISH7070 | GCTTCCGTCTGGACTCCAAC | 20 | AsCpf1 RR | 304 |
| CISH7071 | GACGGAAGCTGGAGTCGGCA | 20 | AsCpf1 RR | 305 |
| CISH7072 | CGCTGTCAGTGAAAACCACT | 20 | AsCpf1 RR | 306 |
| CISH7073 | CTGACAGCGTGAACAGGTAG | 20 | AsCpf1 RR | 307 |
| CISH7074 | TTACGGCCAGCGAGGCCCGA | 20 | AsCpf1 RR | 308 |
| CISH7075 | ATTACGGCCAGCGAGGCCCG | 20 | AsCpf1 RR | 309 |
| CISH7076 | GGAATCTGGTGAGTCTGAGG | 20 | AsCpf1 RR | 310 |
| CISH7077 | CCCTCAGACTCACCAGATTC | 20 | AsCpf1 RR | 311 |
| CISH7078 | CGAAGGTAGGAGAAGGTCTT | 20 | AsCpf1 RR | 312 |
| CISH7079 | GAAGGTAGGAGAAGGTCTTG | 20 | AsCpf1 RR | 313 |
| CISH7080 | GCACCTTTGGCTCACTCTCT | 20 | AsCpf1 RR | 314 |
| CISH7081 | TCGAGGAGGTGGCAGAGGGT | 20 | AsCpf1 RR | 315 |
| CISH7082 | TGGAACTGCCCAAGCCAGTC | 20 | AsCpf1 RR | 316 |
| CISH7083 | AGGGACGGGGCCCACAGGGG | 20 | AsCpf1 RR | 317 |
| CISH7084 | GGGACGGGGCCCACAGGGGC | 20 | AsCpf1 RR | 318 |
| CISH7085 | CTCCACAGCCAGCAAAGGAC | 20 | AsCpf1 RR | 319 |
| CISH7086 | CAGCCAGCAAAGGACGAGGT | 20 | AsCpf1 RR | 320 |
| CISH7087 | CTGCCTTCTAGACCTCGTCC | 20 | AsCpf1 RR | 321 |
| CISH7088 | CCTAAGGAGGATGCGCCTAG | 20 | AsCpf1 RVR | 322 |
| CISH7089 | TGGCCTCCTGCACTGCTGAT | 20 | AsCpf1 RVR | 323 |
| CISH7090 | AGCAGTGCAGGAGGCCACAT | 20 | AsCpf1 RVR | 324 |
| CISH7091 | CCGACTCCAGCTTCCGTCTG | 20 | AsCpf1 RVR | 325 |
| CISH7092 | GGGGTTCCATTACGGCCAGC | 20 | AsCpf1 RVR | 326 |
| CISH7093 | CACAGCAGATCCTCCTCTGG | 20 | AsCpf1 RVR | 327 |
| CISH7094 | ATTGCCCCGTACAGTCAGAG | 20 | SauCas9 | 328 |
| CISH7095 | CCCGTACAGTCAGAGCTGGA | 20 | SauCas9 | 329 |
| CISH7096 | TGGTGGAGGAGCAGGCAGTG | 20 | SauCas9 | 330 |
| CISH7097 | TCCTTAGGCATAGGCAGGGC | 20 | SauCas9 | 331 |
| CISH7098 | CGGCCCTGCCTATGCCTAAG | 20 | SauCas9 | 332 |
| CISH7099 | TAGGCATAGGCAGGGCCGGG | 20 | SauCas9 | 333 |
| CISH7100 | AGGCAGGGCCGGGGTGGGAG | 20 | SauCas9 | 334 |
| CISH7101 | GCAGGATCGGGGCTGTCGCT | 20 | SauCas9 | 335 |
| CISH7102 | CTGCACAAGGCTGACCACAT | 20 | SauCas9 | 336 |
| CISH7103 | TGCACAAGGCTGACCACATC | 20 | SauCas9 | 337 |
| CISH7104 | CTGACCACATCCGGAAAGGC | 20 | SauCas9 | 338 |
| CISH7105 | GGCCACGCATCCTGGCCTTT | 20 | SauCas9 | 339 |
| CISH7106 | GCGTGGCCTGGACAAGCAGT | 20 | SauCas9 | 340 |
| CISH7107 | GACAAGCAGTTGGAGTCCAG | 20 | SauCas9 | 341 |
| CISH7108 | GTTGGAGTCCAGACGGAAGC | 20 | SauCas9 | 342 |
| CISH7109 | ATGCGTACATTGGTGGGGCC | 20 | SauCas9 | 343 |
| CISH7110 | TGGCCCCACCAATGTACGCA | 20 | SauCas9 | 344 |
| CISH7111 | GCTACCTGTTCACGCTGTCA | 20 | SauCas9 | 345 |
| CISH7112 | TGACAGCGTGAACAGGTAGC | 20 | SauCas9 | 346 |
| CISH7113 | GTCGGGCCTCGCTGGCCGTA | 20 | SauCas9 | 347 |
| CISH7114 | GCACTTGCCTAGGCTGGTAT | 20 | SauCas9 | 348 |
| CISH7115 | GGGAATCTGGTGAGTCTGAG | 20 | SauCas9 | 349 |
| CISH7116 | CTCACCAGATTCCCGAAGGT | 20 | SauCas9 | 350 |
| CISH7117 | CTCCTACCTTCGGGAATCTG | 20 | SauCas9 | 351 |
| CISH7118 | CAAGACCTTCTCCTACCTTC | 20 | SauCas9 | 352 |
| CISH7119 | CCAAGACCTTCTCCTACCTT | 20 | SauCas9 | 353 |
| CISH7120 | GCCAAGACCTTCTCCTACCT | 20 | SauCas9 | 354 |
| CISH7121 | TATGCACAGCAGATCCTCCT | 20 | SauCas9 | 355 |
| CISH7122 | CAAAGGTGCTGGACCCAGAG | 20 | SauCas9 | 356 |
| CISH7123 | GGCTCACTCTCTGTCTGGGC | 20 | SauCas9 | 357 |
| CISH7124 | AGGGTACCCCAGCCCAGACA | 20 | SauCas9 | 358 |
| CISH7125 | AGAGGGTACCCCAGCCCAGA | 20 | SauCas9 | 359 |
| CISH7126 | GTACCCTCTGCCACCTCCTC | 20 | SauCas9 | 360 |
| CISH7127 | CCTTCCTCGAGGAGGTGGCA | 20 | SauCas9 | 361 |
| CISH7128 | ATGACTGGCTTGGGCAGTTC | 20 | SauCas9 | 362 |
| CISH7129 | GGCCCCTGTGGGCCCCGTCC | 20 | SauCas9 | 363 |
| CISH7130 | AGGACGAGGTCTAGAAGGCA | 20 | SauCas9 | 364 |
| CISH7131 | ACTGACAGCGTGAACAGGTAG | 21 | Cas12a | 1173 |

In some embodiments, the gRNA for use in the disclosure is a gRNA targeting B2M (B2M gRNA). In some embodiments, the gRNA targeting B2M is one or more of the gRNAs described in Table 9.

TABLE 9
Exemplary B2M gRNAs

| | gRNA Targeting Domain Target | | | SEQ ID |
|---|---|---|---|---|
| gRNA name | sequence (DNA) | Length | Enzyme | NO: |
| B2M1 | TATAAGTGGAGGCGTCGCGC | 20 | SpyCas9 | 365 |
| B2M2 | GGGCACGCGTTTAATATAAG | 20 | SpyCas9 | 366 |
| B2M3 | ACTCACGCTGGATAGCCTCC | 20 | SpyCas9 | 367 |
| B2M4 | GGCCGAGATGTCTCGCTCCG | 20 | SpyCas9 | 368 |
| B2M5 | CACGCGTTTAATATAAGTGG | 20 | SpyCas9 | 369 |
| B2M6 | AAGTGGAGGCGTCGCGCTGG | 20 | SpyCas9 | 370 |
| B2M7 | GAGTAGCGCGAGCACAGCTA | 20 | SpyCas9 | 371 |
| B2M8 | AGTGGAGGCGTCGCGCTGGC | 20 | SpyCas9 | 372 |
| B2M9 | GCCCGAATGCTGTCAGCTTC | 20 | SpyCas9 | 373 |
| B2M10 | CGCGAGCACAGCTAAGGCCA | 20 | SpyCas9 | 374 |
| B2M11 | CTCGCGCTACTCTCTCTTTC | 20 | SpyCas9 | 375 |
| B2M12 | GGCCACGGAGCGAGACATCT | 20 | SpyCas9 | 376 |
| B2M13 | CGTGAGTAAACCTGAATCTT | 20 | SpyCas9 | 377 |
| B2M14 | AGTCACATGGTTCACACGGC | 20 | SpyCas9 | 378 |
| B2M15 | AAGTCAACTTCAATGTCGGA | 20 | SpyCas9 | 379 |
| B2M16 | CAGTAAGTCAACTTCAATGT | 20 | SpyCas9 | 380 |
| B2M17 | ACCCAGACACATAGCAATTC | 20 | SpyCas9 | 381 |
| B2M18 | GCATACTCATCTTTTTCAGT | 20 | SpyCas9 | 382 |
| B2M19 | ACAGCCCAAGATAGTTAAGT | 20 | SpyCas9 | 383 |
| B2M20 | GGCATACTCATCTTTTTCAG | 20 | SpyCas9 | 384 |
| B2M21 | TTCCTGAAGCTGACAGCATT | 20 | SpyCas9 | 385 |
| B2M22 | TCACGTCATCCAGCAGAGAA | 20 | SpyCas9 | 386 |
| B2M23 | CAGCCCAAGATAGTTAAGTG | 20 | SpyCas9 | 387 |
| B2M-c1 | AAUUCUCUCUCCAUUCUU | 18 | AsCpf1 | 388 |
| B2M-c2 | AAUUCUCUCUCCAUUCUUC | 19 | AsCpf1 | 389 |
| B2M-c3 | AAUUCUCUCUCCAUUCUUCA | 20 | AsCpf1 | 390 |
| B2M-c4 | AAUUCUCUCUCCAUUCUUCAG | 21 | AsCpf1 | 391 |
| B2M-c5 | AAUUCUCUCUCCAUUCUUCAGU | 22 | AsCpf1 | 392 |
| B2M-c6 | AAUUCUCUCUCCAUUCUUCAGUA | 23 | AsCpf1 | 393 |
| B2M-c7 | AAUUCUCUCUCCAUUCUUCAGUAA | 24 | AsCpf1 | 394 |
| B2M-c8 | ACUUUCCAUUCUCUGCUG | 18 | AsCpf1 | 395 |
| B2M-c9 | ACUUUCCAUUCUCUGCUGG | 19 | AsCpf1 | 396 |
| B2M-c10 | ACUUUCCAUUCUCUGCUGGA | 20 | AsCpf1 | 397 |
| B2M-c11 | ACUUUCCAUUCUCUGCUGGAU | 21 | AsCpf1 | 398 |
| B2M-c12 | ACUUUCCAUUCUCUGCUGGAUG | 22 | AsCpf1 | 399 |
| B2M-c13 | ACUUUCCAUUCUCUGCUGGAUGA | 23 | AsCpf1 | 400 |
| B2M-c14 | ACUUUCCAUUCUCUGCUGGAUGAC | 24 | AsCpf1 | 401 |
| B2M-c15 | AGCAAGGACUGGUCUUUC | 18 | AsCpf1 | 402 |
| B2M-c16 | AGCAAGGACUGGUCUUUCU | 19 | AsCpf1 | 403 |
| B2M-c17 | AGCAAGGACUGGUCUUUCUA | 20 | AsCpf1 | 404 |
| B2M-c18 | AGCAAGGACUGGUCUUUCUAU | 21 | AsCpf1 | 405 |
| B2M-c19 | AGCAAGGACUGGUCUUUCUAUC | 22 | AsCpf1 | 406 |
| B2M-c20 | AGCAAGGACUGGUCUUUCUAUCU | 23 | AsCpf1 | 407 |
| B2M-c21 | AGCAAGGACUGGUCUUUCUAUCUC | 24 | AsCpf1 | 408 |
| B2M-c22 | AGUGGGGGUGAAUUCAGU | 18 | AsCpf1 | 409 |
| B2M-c23 | AGUGGGGGUGAAUUCAGUG | 19 | AsCpf1 | 410 |
| B2M-c24 | AGUGGGGGUGAAUUCAGUGU | 20 | AsCpf1 | 411 |
| B2M-c25 | AGUGGGGGUGAAUUCAGUGUA | 21 | AsCpf1 | 412 |
| B2M-c26 | AGUGGGGGUGAAUUCAGUGUAG | 22 | AsCpf1 | 413 |
| B2M-c27 | AGUGGGGGUGAAUUCAGUGUAGU | 23 | AsCpf1 | 414 |
| B2M-c28 | AGUGGGGGUGAAUUCAGUGUAGUA | 24 | AsCpf1 | 415 |
| B2M-c29 | AUCCAUCCGACAUUGAAG | 18 | AsCpf1 | 416 |
| B2M-c30 | AUCCAUCCGACAUUGAAGU | 19 | AsCpf1 | 417 |
| B2M-c31 | AUCCAUCCGACAUUGAAGUU | 20 | AsCpf1 | 418 |
| B2M-c32 | AUCCAUCCGACAUUGAAGUUG | 21 | AsCpf1 | 419 |
| B2M-c33 | AUCCAUCCGACAUUGAAGUUGA | 22 | AsCpf1 | 420 |
| B2M-c34 | AUCCAUCCGACAUUGAAGUUGAC | 23 | AsCpf1 | 421 |
| B2M-c35 | AUCCAUCCGACAUUGAAGUUGACU | 24 | AsCpf1 | 422 |
| B2M-c36 | CAAUUCUCUCUCCAUUCU | 18 | AsCpf1 | 423 |
| B2M-c37 | CAAUUCUCUCUCCAUUCUU | 19 | AsCpf1 | 424 |
| B2M-c38 | CAAUUCUCUCUCCAUUCUUC | 20 | AsCpf1 | 425 |
| B2M-c39 | CAAUUCUCUCUCCAUUCUUCA | 21 | AsCpf1 | 426 |
| B2M-c40 | CAAUUCUCUCUCCAUUCUUCAG | 22 | AsCpf1 | 427 |
| B2M-c41 | CAAUUCUCUCUCCAUUCUUCAGU | 23 | AsCpf1 | 428 |
| B2M-c42 | CAAUUCUCUCUCCAUUCUUCAGUA | 24 | AsCpf1 | 429 |
| B2M-c43 | CAGUGGGGGUGAAUUCAG | 18 | AsCpf1 | 430 |
| B2M-c44 | CAGUGGGGGUGAAUUCAGU | 19 | AsCpf1 | 431 |
| B2M-c45 | CAGUGGGGGUGAAUUCAGUG | 20 | AsCpf1 | 432 |
| B2M-c46 | CAGUGGGGGUGAAUUCAGUGU | 21 | AsCpf1 | 433 |
| B2M-c47 | CAGUGGGGGUGAAUUCAGUGUA | 22 | AsCpf1 | 434 |
| B2M-c48 | CAGUGGGGGUGAAUUCAGUGUAG | 23 | AsCpf1 | 435 |
| B2M-c49 | CAGUGGGGGUGAAUUCAGUGUAGU | 24 | AsCpf1 | 436 |
| B2M-c50 | CAUUCUCUGCUGGAUGAC | 18 | AsCpf1 | 437 |
| B2M-c51 | CAUUCUCUGCUGGAUGACG | 19 | AsCpf1 | 438 |
| B2M-c52 | CAUUCUCUGCUGGAUGACGU | 20 | AsCpf1 | 439 |
| B2M-c53 | CAUUCUCUGCUGGAUGACGUG | 21 | AsCpf1 | 440 |
| B2M-c54 | CAUUCUCUGCUGGAUGACGUGA | 22 | AsCpf1 | 44 |
| B2M-c55 | CAUUCUCUGCUGGAUGACGUGAG | 23 | AsCpf1 | 442 |
| B2M-c56 | CAUUCUCUGCUGGAUGACGUGAGU | 24 | AsCpf1 | 443 |
| B2M-c57 | CCCGAUAUUCCUCAGGUA | 18 | AsCpf1 | 444 |
| B2M-c58 | CCCGAUAUUCCUCAGGUAC | 19 | AsCpf1 | 445 |
| B2M-c59 | CCCGAUAUUCCUCAGGUACU | 20 | AsCpf1 | 446 |
| B2M-c60 | CCCGAUAUUCCUCAGGUACUC | 21 | AsCpf1 | 447 |
| B2M-c61 | CCCGAUAUUCCUCAGGUACUCC | 22 | AsCpf1 | 448 |
| B2M-c62 | CCCGAUAUUCCUCAGGUACUCCA | 23 | AsCpf1 | 449 |
| B2M-c63 | CCCGAUAUUCCUCAGGUACUCCAA | 24 | AsCpf1 | 450 |
| B2M-c64 | CCGAUAUUCCUCAGGUAC | 18 | AsCpf1 | 451 |
| B2M-c65 | CCGAUAUUCCUCAGGUACU | 19 | AsCpf1 | 452 |
| B2M-c66 | CCGAUAUUCCUCAGGUACUC | 20 | AsCpf1 | 453 |
| B2M-c67 | CCGAUAUUCCUCAGGUACUCC | 21 | AsCpf1 | 454 |
| B2M-c68 | CCGAUAUUCCUCAGGUACUCCA | 22 | AsCpf1 | 455 |
| B2M-c69 | CCGAUAUUCCUCAGGUACUCCAA | 23 | AsCpf1 | 456 |
| B2M-c70 | CCGAUAUUCCUCAGGUACUCCAAA | 24 | AsCpf1 | 457 |
| B2M-c71 | CUCACGUCAUCCAGCAGA | 18 | AsCpf1 | 458 |
| B2M-c72 | CUCACGUCAUCCAGCAGAG | 19 | AsCpf1 | 459 |
| B2M-c73 | CUCACGUCAUCCAGCAGAGA | 20 | AsCpf1 | 460 |
| B2M-c74 | CUCACGUCAUCCAGCAGAGAA | 21 | AsCpf1 | 461 |
| B2M-c75 | CUCACGUCAUCCAGCAGAGAAU | 22 | AsCpf1 | 462 |
| B2M-c76 | CUCACGUCAUCCAGCAGAGAAUG | 23 | AsCpf1 | 463 |
| B2M-c77 | CUCACGUCAUCCAGCAGAGAAUGG | 24 | AsCpf1 | 464 |
| B2M-c78 | CUGAAUUGCUAUGUGUCU | 18 | AsCpf1 | 465 |
| B2M-c79 | CUGAAUUGCUAUGUGUCUG | 19 | AsCpf1 | 466 |
| B2M-c80 | CUGAAUUGCUAUGUGUCUGG | 20 | AsCpf1 | 467 |
| B2M-c81 | CUGAAUUGCUAUGUGUCUGGG | 21 | AsCpf1 | 468 |
| B2M-c82 | CUGAAUUGCUAUGUGUCUGGGU | 22 | AsCpf1 | 469 |
| B2M-c83 | CUGAAUUGCUAUGUGUCUGGGUU | 23 | AsCpf1 | 470 |
| B2M-c84 | CUGAAUUGCUAUGUGUCUGGGUUU | 24 | AsCpf1 | 471 |
| B2M-c85 | GAGUACCUGAGGAAUAUC | 18 | AsCpf1 | 472 |
| B2M-c86 | GAGUACCUGAGGAAUAUCG | 19 | AsCpf1 | 473 |
| B2M-c87 | GAGUACCUGAGGAAUAUCGG | 20 | AsCpf1 | 474 |
| B2M-c88 | GAGUACCUGAGGAAUAUCGGG | 21 | AsCpf1 | 475 |
| B2M-c89 | GAGUACCUGAGGAAUAUCGGGA | 22 | AsCpf1 | 476 |
| B2M-c90 | GAGUACCUGAGGAAUAUCGGGAA | 23 | AsCpf1 | 477 |
| B2M-c91 | GAGUACCUGAGGAAUAUCGGGAAA | 24 | AsCpf1 | 478 |
| B2M-c92 | UAUCUCUUGUACUACACU | 18 | AsCpf1 | 479 |
| B2M-c93 | UAUCUCUUGUACUACACUG | 19 | AsCpf1 | 480 |
| B2M-c94 | UAUCUCUUGUACUACACUGA | 20 | AsCpf1 | 481 |
| B2M-c95 | UAUCUCUUGUACUACACUGAA | 21 | AsCpf1 | 482 |
| B2M-c96 | UAUCUCUUGUACUACACUGAAU | 22 | AsCpf1 | 483 |
| B2M-c97 | UAUCUCUUGUACUACACUGAAUU | 23 | AsCpf1 | 484 |
| B2M-c98 | UAUCUCUUGUACUACACUGAAUUC | 24 | AsCpf1 | 485 |
| B2M-c99 | UCAAUUCUCUCUCCAUUC | 18 | AsCpf1 | 486 |
| B2M-c100 | UCAAUUCUCUCUCCAUUCU | 19 | AsCpf1 | 487 |
| B2M-c101 | UCAAUUCUCUCUCCAUUCUU | 20 | AsCpf1 | 488 |
| B2M-c102 | UCAAUUCUCUCUCCAUUCUUC | 21 | AsCpf1 | 489 |
| B2M-c103 | UCAAUUCUCUCUCCAUUCUUCA | 22 | AsCpf1 | 490 |
| B2M-c104 | UCAAUUCUCUCUCCAUUCUUCAG | 23 | AsCpf1 | 491 |
| B2M-c105 | UCAAUUCUCUCUCCAUUCUUCAGU | 24 | AsCpf1 | 492 |
| B2M-c106 | UCACAGCCCAAGAUAGUU | 18 | AsCpf1 | 493 |
| B2M-c107 | UCACAGCCCAAGAUAGUUA | 19 | AsCpf1 | 494 |
| B2M-c108 | UCACAGCCCAAGAUAGUUAA | 20 | AsCpf1 | 495 |
| B2M-c109 | UCACAGCCCAAGAUAGUUAAG | 21 | AsCpf1 | 496 |
| B2M-c110 | UCACAGCCCAAGAUAGUUAAGU | 22 | AsCpf1 | 497 |
| B2M-c111 | UCACAGCCCAAGAUAGUUAAGUG | 23 | AsCpf1 | 498 |
| B2M-c112 | UCACAGCCCAAGAUAGUUAAGUGG | 24 | AsCpf1 | 499 |
| B2M-c113 | UCAGUGGGGGUGAAUUCA | 18 | AsCpf1 | 500 |
| B2M-c114 | UCAGUGGGGGUGAAUUCAG | 19 | AsCpf1 | 501 |
| B2M-c115 | UCAGUGGGGGUGAAUUCAGU | 20 | AsCpf1 | 502 |
| B2M-c116 | UCAGUGGGGGUGAAUUCAGUG | 21 | AsCpf1 | 503 |
| B2M-c117 | UCAGUGGGGGUGAAUUCAGUGU | 22 | AsCpf1 | 504 |
| B2M-c118 | UCAGUGGGGGUGAAUUCAGUGUA | 23 | AsCpf1 | 505 |
| B2M-c119 | UCAGUGGGGGUGAAUUCAGUGUAG | 24 | AsCpf1 | 506 |
| B2M-c120 | UGGCCUGGAGGCUAUCCA | 18 | AsCpf1 | 507 |
| B2M-c121 | UGGCCUGGAGGCUAUCCAG | 19 | AsCpf1 | 508 |
| B2M-c122 | UGGCCUGGAGGCUAUCCAGC | 20 | AsCpf1 | 509 |
| B2M-c123 | UGGCCUGGAGGCUAUCCAGCG | 21 | AsCpf1 | 510 |
| B2M-c124 | UGGCCUGGAGGCUAUCCAGCGU | 22 | AsCpf1 | 511 |
| B2M-c125 | UGGCCUGGAGGCUAUCCAGCGUG | 23 | AsCpf1 | 512 |
| B2M-c126 | UGGCCUGGAGGCUAUCCAGCGUGA | 24 | AsCpf1 | 513 |
| B2M-c127 | AUAGAUCGAGACAUGUAA | 18 | AsCpf1 | 514 |
| B2M-c128 | AUAGAUCGAGACAUGUAAG | 19 | AsCpf1 | 515 |
| B2M-c129 | AUAGAUCGAGACAUGUAAGC | 20 | AsCpf1 | 516 |
| B2M-c130 | AUAGAUCGAGACAUGUAAGCA | 21 | AsCpf1 | 517 |
| B2M-c131 | AUAGAUCGAGACAUGUAAGCAG | 22 | AsCpf1 | 518 |
| B2M-c132 | AUAGAUCGAGACAUGUAAGCAGC | 23 | AsCpf1 | 519 |
| B2M-c133 | AUAGAUCGAGACAUGUAAGCAGCA | 24 | AsCpf1 | 520 |
| B2M-c134 | CAUAGAUCGAGACAUGUA | 18 | AsCpf1 | 521 |
| B2M-c135 | CAUAGAUCGAGACAUGUAA | 19 | AsCpf1 | 522 |
| B2M-c136 | CAUAGAUCGAGACAUGUAAG | 20 | AsCpf1 | 523 |
| B2M-c137 | CAUAGAUCGAGACAUGUAAGC | 21 | AsCpf1 | 524 |
| B2M-c138 | CAUAGAUCGAGACAUGUAAGCA | 22 | AsCpf1 | 525 |
| B2M-c139 | CAUAGAUCGAGACAUGUAAGCAG | 23 | AsCpf1 | 526 |
| B2M-c140 | CAUAGAUCGAGACAUGUAAGCAGC | 24 | AsCpf1 | 527 |
| B2M-c141 | CUCCACUGUCUUUUUCAU | 18 | AsCpf1 | 528 |
| B2M-c142 | CUCCACUGUCUUUUUCAUA | 19 | AsCpf1 | 529 |
| B2M-c143 | CUCCACUGUCUUUUUCAUAG | 20 | AsCpf1 | 530 |
| B2M-c144 | CUCCACUGUCUUUUUCAUAGA | 21 | AsCpf1 | 531 |
| B2M-c145 | CUCCACUGUCUUUUUCAUAGAU | 22 | AsCpf1 | 532 |
| B2M-c146 | CUCCACUGUCUUUUUCAUAGAUC | 23 | AsCpf1 | 533 |
| B2M-c147 | CUCCACUGUCUUUUUCAUAGAUCG | 24 | AsCpf1 | 534 |
| B2M-c148 | UCAUAGAUCGAGACAUGU | 18 | AsCpf1 | 535 |
| B2M-c149 | UCAUAGAUCGAGACAUGUA | 19 | AsCpf1 | 536 |
| B2M-c150 | UCAUAGAUCGAGACAUGUAA | 20 | AsCpf1 | 537 |
| B2M-c151 | UCAUAGAUCGAGACAUGUAAG | 21 | AsCpf1 | 538 |
| B2M-c152 | UCAUAGAUCGAGACAUGUAAGC | 22 | AsCpf1 | 539 |
| B2M-c153 | UCAUAGAUCGAGACAUGUAAGCA | 23 | AsCpf1 | 540 |
| B2M-c154 | UCAUAGAUCGAGACAUGUAAGCAG | 24 | AsCpf1 | 541 |
| B2M-c155 | UCCACUGUCUUUUUCAUA | 18 | AsCpf1 | 542 |
| B2M-c156 | UCCACUGUCUUUUUCAUAG | 19 | AsCpf1 | 543 |
| B2M-c157 | UCCACUGUCUUUUUCAUAGA | 20 | AsCpf1 | 544 |
| B2M-c158 | UCCACUGUCUUUUUCAUAGAU | 21 | AsCpf1 | 545 |
| B2M-c159 | UCCACUGUCUUUUUCAUAGAUC | 22 | AsCpf1 | 546 |
| B2M-c160 | UCCACUGUCUUUUUCAUAGAUCG | 23 | AsCpf1 | 547 |
| B2M-c161 | UCCACUGUCUUUUUCAUAGAUCGA | 24 | AsCpf1 | 548 |
| B2M-c162 | UCUCCACUGUCUUUUUCA | 18 | AsCpf1 | 549 |
| B2M-c163 | UCUCCACUGUCUUUUUCAU | 19 | AsCpf1 | 550 |
| B2M-c164 | UCUCCACUGUCUUUUUCAUA | 20 | AsCpf1 | 551 |
| B2M-c165 | UCUCCACUGUCUUUUUCAUAG | 21 | AsCpf1 | 552 |
| B2M-c166 | UCUCCACUGUCUUUUUCAUAGA | 22 | AsCpf1 | 553 |
| B2M-c167 | UCUCCACUGUCUUUUUCAUAGAU | 23 | AsCpf1 | 554 |
| B2M-c168 | UCUCCACUGUCUUUUUCAUAGAUC | 24 | AsCpf1 | 555 |
| B2M-c169 | UUCUCCACUGUCUUUUUC | 18 | AsCpf1 | 556 |
| B2M-c170 | UUCUCCACUGUCUUUUUCA | 19 | AsCpf1 | 557 |
| B2M-c171 | UUCUCCACUGUCUUUUUCAU | 20 | AsCpf1 | 558 |
| B2M-c172 | UUCUCCACUGUCUUUUUCAUA | 21 | AsCpf1 | 559 |
| B2M-c173 | UUCUCCACUGUCUUUUUCAUAG | 22 | AsCpf1 | 560 |
| B2M-c174 | UUCUCCACUGUCUUUUUCAUAGA | 23 | AsCpf1 | 561 |
| B2M-c175 | UUCUCCACUGUCUUUUUCAUAGAU | 24 | AsCpf1 | 562 |
| B2M-c176 | UUUCUCCACUGUCUUUUU | 18 | AsCpf1 | 563 |
| B2M-c177 | UUUCUCCACUGUCUUUUUC | 19 | AsCpf1 | 564 |
| B2M-c178 | UUUCUCCACUGUCUUUUUCA | 20 | AsCpf1 | 565 |
| B2M-c179 | UUUCUCCACUGUCUUUUUCAU | 21 | AsCpf1 | 566 |
| B2M-c180 | UUUCUCCACUGUCUUUUUCAUA | 22 | AsCpf1 | 567 |
| B2M-c181 | UUUCUCCACUGUCUUUUUCAUAG | 23 | AsCpf1 | 568 |
| B2M-c182 | UUUCUCCACUGUCUUUUUCAUAGA | 24 | AsCpf1 | 569 |
| B2M-c183 | UUUUCUCCACUGUCUUUU | 18 | AsCpf1 | 570 |
| B2M-c184 | UUUUCUCCACUGUCUUUUU | 19 | AsCpf1 | 571 |
| B2M-c185 | UUUUCUCCACUGUCUUUUUC | 20 | AsCpf1 | 572 |
| B2M-c186 | UUUUCUCCACUGUCUUUUUCA | 21 | AsCpf1 | 573 |
| B2M-c187 | UUUUCUCCACUGUCUUUUUCAU | 22 | AsCpf1 | 574 |
| B2M-c188 | UUUUCUCCACUGUCUUUUUCAUA | 23 | AsCpf1 | 575 |
| B2M-c189 | UUUUCUCCACUGUCUUUUUCAUAG | 24 | AsCpf1 | 576 |

In some embodiments, the gRNA for use in the disclosure is a gRNA targeting PD1. gRNAs targeting B2M and PD1 for use in the disclosure are further described in WO2015161276 and WO2017152015 by Welstead et al.; both incorporated in their entirety herein by reference.

In some embodiments, the gRNA for use in the disclosure is a gRNA targeting NKG2A (NKG2A gRNA). In some embodiments, the gRNA targeting NKG2A is one or more of the gRNAs described in Table 10.

TABLE 10
Exemplary NKG2A gRNAs

| | gRNA Targeting Domain | | | SEQ ID |
|---|---|---|---|---|
| Name | Sequence (DNA) | Length | Enzyme | NO: |
| NKG2A55 | GAGGTAAAGCGTTTGCATTTG | 21 | AsCpf1 | 577 |
| NKG2A56 | CCTCTAAAGCTTATGCTTACA | 21 | AsCpf1 | 578 |
| NKG2A57 | AGTCGATTTACTTGTAGCACT | 21 | AsCpf1 | 579 |
| NKG2A58 | CTTGTAGCACTGCACAGTTAA | 21 | AsCpf1 | 580 |
| NKG2A59 | TCCATTACAGGATAAAAGACT | 21 | AsCpf1 | 581 |
| NKG2A60 | CTCCATTACAGGATAAAAGAC | 21 | AsCpf1 | 582 |
| NKG2A61 | TCTCCATTACAGGATAAAAGA | 21 | AsCpf1 | 583 |
| NKG2A62 | ATCCTGTAATGGAGAAAAATC | 21 | AsCpf1 | 584 |
| NKG2A63 | TCCTGTAATGGAGAAAAATCC | 21 | AsCpf1 | 585 |
| NKG2A136 | AAACATGAGTAAGTTGTTTTG | 21 | AsCpf1 | 586 |
| NKG2A137 | GCTTTCAAACATGAGTAAGTT | 21 | AsCpf1 | 587 |
| NKG2A138 | AAAGCCAAACCATTCATTGTC | 21 | AsCpf1 | 588 |
| NKG2A139 | GTAACAGCAGTCATCATCCAT | 21 | AsCpf1 | 589 |
| NKG2A140 | ACCATCCTCATGGATTGGTGT | 21 | AsCpf1 | 590 |
| NKG2A141 | TGTCCATCATTTCACCATCCT | 21 | AsCpf1 | 591 |
| NKG2A142 | GAAATTTCTGTCCATCATTTC | 21 | AsCpf1 | 592 |
| NKG2A143 | AGAAATTTCTGTCCATCATTT | 21 | AsCpf1 | 593 |
| NKG2A144 | TTTTAGAAATTTCTGTCCATC | 21 | AsCpf1 | 594 |
| NKG2A145 | CTTTTAGAAATTTCTGTCCAT | 21 | AsCpf1 | 595 |
| NKG2A146 | TTTTCTTTTAGAAATTTCTGT | 21 | AsCpf1 | 596 |
| NKG2A147 | TAAAAGAAAAGAAAGAATTTT | 21 | AsCpf1 | 597 |
| NKG2A270 | AAACATTTACATCTTACCATT | 21 | AsCpf1 | 598 |
| NKG2A271 | CATCTTACCATTTCTTCTTCA | 21 | AsCpf1 | 599 |
| NKG2A272 | TATAGATAATGAAGAAGAAAT | 21 | AsCpf1 | 600 |
| NKG2A273 | TTCTTCATTATCTATAGAAAG | 21 | AsCpf1 | 601 |
| NKG2A274 | CTGGCCTGTACTTCGAAGAAC | 21 | AsCpf1 | 602 |
| NKG2A275 | CTTACCAATGTAGTAACAACT | 21 | AsCpf1 | 603 |
| NKG2A276 | GCACGTCATTGTGGCCATTGT | 21 | AsCpf1 | 604 |
| NKG2A277 | TTTAGCACGTCATTGTGGCCA | 21 | AsCpf1 | 605 |
| NKG2A414 | CCATCAGCTCCAGAGAAGCTC | 21 | AsCpf1 | 606 |
| NKG2A415 | TCTCCCTGCAGATTTACCATC | 21 | AsCpf1 | 607 |
| NKG2A437 | AAATGCTTTACCTTTGCAGTG | 21 | AsCpf1 | 608 |
| NKG2A438 | AATGCTTTACCTTTGCAGTGA | 21 | AsCpf1 | 609 |
| NKG2A439 | CCTTTGCAGTGATAGGTTTTG | 21 | AsCpf1 | 610 |
| NKG2A440 | CAGTGATAGGTTTTGTCATTC | 21 | AsCpf1 | 611 |
| NKG2A441 | AAGGGAATGACAAAACCTATC | 21 | AsCpf1 | 612 |
| NKG2A442 | CAAGGGAATGACAAAACCTAT | 21 | AsCpf1 | 613 |
| NKG2A443 | GTCATTCCCTTGAAAATCCTG | 21 | AsCpf1 | 614 |
| NKG2A444 | TCATTCCCTTGAAAATCCTGA | 21 | AsCpf1 | 615 |
| NKG2A445 | TGAAGGTTTAATTCCGCATAG | 21 | AsCpf1 | 616 |
| NKG2A446 | GAAGGTTTAATTCCGCATAGG | 21 | AsCpf1 | 617 |
| NKG2A447 | AAGGTTTAATTCCGCATAGGT | 21 | AsCpf1 | 618 |
| NKG2A448 | ATTCCGCATAGGTTATTTCCT | 21 | AsCpf1 | 619 |
| NKG2A449 | GCAACTGAACAGGAAATAACC | 21 | AsCpf1 | 620 |
| NKG2A450 | AGCAACTGAACAGGAAATAAC | 21 | AsCpf1 | 621 |
| NKG2A451 | CTGTTCAGTTGCTAAAATGGA | 21 | AsCpf1 | 622 |
| NKG2A452 | TATTGCCTTTAGGTTTTCGTT | 21 | AsCpf1 | 623 |
| NKG2A453 | ATTGCCTTTAGGTTTTCGTTG | 21 | AsCpf1 | 624 |
| NKG2A454 | TTGCCTTTAGGTTTTCGTTGC | 21 | AsCpf1 | 625 |
| NKG2A455 | GGTTTTCGTTGCTGCCTCTTT | 21 | AsCpf1 | 626 |
| NKG2A456 | CGTTGCTGCCTCTTTGGGTTT | 21 | AsCpf1 | 627 |
| NKG2A457 | GTTGCTGCCTCTTTGGGTTTG | 21 | AsCpf1 | 628 |
| NKG2A458 | GGTTTGGGGGCAGATTCAGGT | 21 | AsCpf1 | 629 |
| NKG2A459 | GGGGCAGATTCAGGTCTGAGT | 21 | AsCpf1 | 630 |
| NKG2A460 | GCAACTGAACAGGAAATAACC | 21 | Cas12a | 1176 |

In some embodiments, the gRNA for use in the disclosure is a gRNA targeting TIGIT (TIGIT gRNA). In some embodiments, the gRNA targeting TIGIT is one or more of the gRNAs described in Table 11.

TABLE 11
Exemplary TIGIT gRNAs

| | gRNA Targeting Domain | | | SEQ ID |
|---|---|---|---|---|
| Name | Sequence (DNA) | Length | Enzyme | NO: |
| TIGIT4170 | TCTGCAGAAATGTTCCCCGT | 20 | AsCpf1 | 631 |
| TIGIT4171 | TGCAGAGAAAGGTGGCTCTA | 20 | AsCpf1 | 632 |
| TIGIT4172 | TAATGCTGACTTGGGGTGGC | 20 | AsCpf1 | 633 |
| TIGIT4173 | TAGGACCTCCAGGAAGATTC | 20 | AsCpf1 | 634 |
| TIGIT4174 | TAGTCAACGCGACCACCACG | 20 | AsCpf1 | 635 |
| TIGIT4175 | TCCTGAGGTCACCTTCCACA | 20 | AsCpf1 | 636 |
| TIGIT4176 | TATTGTGCCTGTCATCATTC | 20 | AsCpf1 | 637 |
| TIGIT4177 | TGACAGGCACAATAGAAACAA | 21 | SauCas9 | 638 |
| TIGIT4178 | GACAGGCACAATAGAAACAAC | 21 | SauCas9 | 639 |
| TIGIT4179 | AAACAACGGGGAACATTTCTG | 21 | SauCas9 | 640 |
| TIGIT4180 | ACAACGGGGAACATTTCTGCA | 21 | SauCas9 | 641 |
| TIGIT4181 | TGATAGAGCCACCTTTCTCTG | 21 | SauCas9 | 642 |
| TIGIT4182 | GGGTCACTTGTGCCGTGGTGG | 21 | SauCas9 | 643 |
| TIGIT4183 | GGCACAAGTGACCCAGGTCAA | 21 | SauCas9 | 644 |
| TIGIT4184 | GTCCTGCTGCTCCCAGTTGAC | 21 | SauCas9 | 645 |
| TIGIT4185 | TGGCCATTTGTAATGCTGACT | 21 | SauCas9 | 646 |
| TIGIT4186 | TGGCACATCTCCCCATCCTTC | 21 | SauCas9 | 647 |
| TIGIT4187 | CATCTCCCCATCCTTCAAGGA | 21 | SauCas9 | 648 |
| TIGIT4188 | CCACTCGATCCTTGAAGGATG | 21 | SauCas9 | 649 |
| TIGIT4189 | GGCCACTCGATCCTTGAAGGA | 21 | SauCas9 | 650 |
| TIGIT4190 | CCTGGGGCCACTCGATCCTTG | 21 | SauCas9 | 651 |
| TIGIT4191 | GACTGGAGGGTGAGGCCCAGG | 21 | SauCas9 | 652 |
| TIGIT4192 | ATCGTTCACGGTCAGCGACTG | 21 | SauCas9 | 653 |
| TIGIT4193 | GTCGCTGACCGTGAACGATAC | 21 | SauCas9 | 654 |
| TIGIT4194 | CGCTGACCGTGAACGATACAG | 21 | SauCas9 | 655 |
| TIGIT4195 | GCATCTATCACACCTACCCTG | 21 | SauCas9 | 656 |
| TIGIT4196 | CCTACCCTGATGGGACGTACA | 21 | SauCas9 | 657 |
| TIGIT4197 | TACCCTGATGGGACGTACACT | 21 | SauCas9 | 658 |
| TIGIT4198 | CCCTGATGGGACGTACACTGG | 21 | SauCas9 | 659 |
| TIGIT4199 | TTCTCCCAGTGTACGTCCCAT | 21 | SauCas9 | 660 |
| TIGIT4200 | GGAGAATCTTCCTGGAGGTCC | 21 | SauCas9 | 661 |
| TIGIT4201 | CATGGCTCCAAGCAATGGAAT | 21 | SauCas9 | 662 |
| TIGIT4202 | CGCGGCCATGGCTCCAAGCAA | 21 | SauCas9 | 663 |
| TIGIT4203 | TCGCGGCCATGGCTCCAAGCA | 21 | SauCas9 | 664 |
| TIGIT4204 | CATCGTGGTGGTCGCGTTGAC | 21 | SauCas9 | 665 |
| TIGIT4205 | AAAGCCCTCAGAATCCATTCT | 21 | SauCas9 | 666 |
| TIGIT4206 | CATTCTGTGGAAGGTGACCTC | 21 | SauCas9 | 667 |
| TIGIT4207 | TTCTGTGGAAGGTGACCTCAG | 21 | SauCas9 | 668 |
| TIGIT4208 | CCTGAGGTCACCTTCCACAGA | 21 | SauCas9 | 669 |
| TIGIT4209 | TTCTCCTGAGGTCACCTTCCA | 21 | SauCas9 | 670 |
| TIGIT4210 | AGGAGAAAATCAGCTGGACAG | 21 | SauCas9 | 671 |
| TIGIT4211 | GGAGAAAATCAGCTGGACAGG | 21 | SauCas9 | 672 |
| TIGIT4212 | GCCCCAGTGCTCCCTCACCCC | 21 | SauCas9 | 673 |
| TIGIT4213 | TGGACACAGCTTCCTGGGGGT | 21 | SauCas9 | 674 |
| TIGIT4214 | TCTGCCTGGACACAGCTTCCT | 21 | SauCas9 | 675 |
| TIGIT4215 | AGCTGCACCTGCTGGGCTCTG | 21 | SauCas9 | 676 |
| TIGIT4216 | GCTGGGCTCTGTGGAGAGCAG | 21 | SauCas9 | 677 |
| TIGIT4217 | TGGGCTCTGTGGAGAGCAGCG | 21 | SauCas9 | 678 |
| TIGIT4218 | CTGCATGACTACTTCAATGTC | 21 | SauCas9 | 679 |
| TIGIT4219 | AATGTCCTGAGTTACAGAAGC | 21 | SauCas9 | 680 |
| TIGIT4220 | TGGGTAACTGCAGCTTCTTCA | 21 | SauCas9 | 681 |
| TIGIT4221 | GACAGGCACAATAGAAACAA | 20 | SpyCas9 | 682 |
| TIGIT4222 | ACAGGCACAATAGAAACAAC | 20 | SpyCas9 | 683 |
| TIGIT4223 | CAGGCACAATAGAAACAACG | 20 | SpyCas9 | 684 |
| TIGIT4224 | GGGAACATTTCTGCAGAGAA | 20 | SpyCas9 | 685 |
| TIGIT4225 | AACATTTCTGCAGAGAAAGG | 20 | SpyCas9 | 686 |
| TIGIT4226 | ATGTCACCTCTCCTCCACCA | 20 | SpyCas9 | 687 |
| TIGIT4227 | CTTGTGCCGTGGTGGAGGAG | 20 | SpyCas9 | 688 |
| TIGIT4228 | GGTCACTTGTGCCGTGGTGG | 20 | SpyCas9 | 689 |
| TIGIT4229 | CACCACGGCACAAGTGACCC | 20 | SpyCas9 | 690 |
| TIGIT4230 | CTGGGTCACTTGTGCCGTGG | 20 | SpyCas9 | 691 |
| TIGIT4231 | GACCTGGGTCACTTGTGCCG | 20 | SpyCas9 | 692 |
| TIGIT4232 | CACAAGTGACCCAGGTCAAC | 20 | SpyCas9 | 693 |
| TIGIT4233 | ACAAGTGACCCAGGTCAACT | 20 | SpyCas9 | 694 |
| TIGIT4234 | CCAGGTCAACTGGGAGCAGC | 20 | SpyCas9 | 695 |
| TIGIT4235 | CTGCTGCTCCCAGTTGACCT | 20 | SpyCas9 | 696 |
| TIGIT4236 | CCTGCTGCTCCCAGTTGACC | 20 | SpyCas9 | 697 |
| TIGIT4237 | GGAGCAGCAGGACCAGCTTC | 20 | SpyCas9 | 698 |
| TIGIT4238 | CATTACAAATGGCCAGAAGC | 20 | SpyCas9 | 699 |
| TIGIT4239 | GGCCATTTGTAATGCTGACT | 20 | SpyCas9 | 700 |
| TIGIT4240 | GCCATTTGTAATGCTGACTT | 20 | SpyCas9 | 701 |
| TIGIT4241 | CCATTTGTAATGCTGACTTG | 20 | SpyCas9 | 702 |
| TIGIT4242 | TTTGTAATGCTGACTTGGGG | 20 | SpyCas9 | 703 |
| TIGIT4243 | CCCCAAGTCAGCATTACAAA | 20 | SpyCas9 | 704 |
| TIGIT4244 | GCACATCTCCCCATCCTTCA | 20 | SpyCas9 | 705 |
| TIGIT4245 | CCCATCCTTCAAGGATCGAG | 20 | SpyCas9 | 706 |
| TIGIT4246 | CACTCGATCCTTGAAGGATG | 20 | SpyCas9 | 707 |
| TIGIT4247 | CCACTCGATCCTTGAAGGAT | 20 | SpyCas9 | 708 |
| TIGIT4248 | GCCACTCGATCCTTGAAGGA | 20 | SpyCas9 | 709 |
| TIGIT4249 | TTCAAGGATCGAGTGGCCCC | 20 | SpyCas9 | 710 |
| TIGIT4250 | TGGGGCCACTCGATCCTTGA | 20 | SpyCas9 | 711 |
| TIGIT4251 | GATCGAGTGGCCCCAGGTCC | 20 | SpyCas9 | 712 |
| TIGIT4252 | AGTGGCCCCAGGTCCCGGCC | 20 | SpyCas9 | 713 |
| TIGIT4253 | GTGGCCCCAGGTCCCGGCCT | 20 | SpyCas9 | 714 |
| TIGIT4254 | GAGGCCCAGGCCGGGACCTG | 20 | SpyCas9 | 715 |
| TIGIT4255 | TGAGGCCCAGGCCGGGACCT | 20 | SpyCas9 | 716 |
| TIGIT4256 | GTGAGGCCCAGGCCGGGACC | 20 | SpyCas9 | 717 |
| TIGIT4257 | TGGAGGGTGAGGCCCAGGCC | 20 | SpyCas9 | 718 |
| TIGIT4258 | CTGGAGGGTGAGGCCCAGGC | 20 | SpyCas9 | 719 |
| TIGIT4259 | GCGACTGGAGGGTGAGGCCC | 20 | SpyCas9 | 720 |
| TIGIT4260 | CGGTCAGCGACTGGAGGGTG | 20 | SpyCas9 | 721 |
| TIGIT4261 | GTTCACGGTCAGCGACTGGA | 20 | SpyCas9 | 722 |
| TIGIT4262 | CGTTCACGGTCAGCGACTGG | 20 | SpyCas9 | 723 |
| TIGIT4263 | TATCGTTCACGGTCAGCGAC | 20 | SpyCas9 | 724 |
| TIGIT4264 | TCGCTGACCGTGAACGATAC | 20 | SpyCas9 | 725 |
| TIGIT4265 | CGCTGACCGTGAACGATACA | 20 | SpyCas9 | 726 |
| TIGIT4266 | GCTGACCGTGAACGATACAG | 20 | SpyCas9 | 727 |
| TIGIT4267 | GTACTCCCCTGTATCGTTCA | 20 | SpyCas9 | 728 |
| TIGIT4268 | ATCTATCACACCTACCCTGA | 20 | SpyCas9 | 729 |
| TIGIT4269 | TCTATCACACCTACCCTGAT | 20 | SpyCas9 | 730 |
| TIGIT4270 | TACCCTGATGGGACGTACAC | 20 | SpyCas9 | 731 |
| TIGIT4271 | ACCCTGATGGGACGTACACT | 20 | SpyCas9 | 732 |
| TIGIT4272 | AGTGTACGTCCCATCAGGGT | 20 | SpyCas9 | 733 |
| TIGIT4273 | TCCCAGTGTACGTCCCATCA | 20 | SpyCas9 | 734 |
| TIGIT4274 | CTCCCAGTGTACGTCCCATC | 20 | SpyCas9 | 735 |
| TIGIT4275 | GTACACTGGGAGAATCTTCC | 20 | SpyCas9 | 736 |
| TIGIT4276 | CACTGGGAGAATCTTCCTGG | 20 | SpyCas9 | 737 |
| TIGIT4277 | CTGAGCTTTCTAGGACCTCC | 20 | SpyCas9 | 738 |
| TIGIT4278 | AGGTTCCAGATTCCATTGCT | 20 | SpyCas9 | 739 |
| TIGIT4279 | AAGCAATGGAATCTGGAACC | 20 | SpyCas9 | 740 |
| TIGIT4280 | GATTCCATTGCTTGGAGCCA | 20 | SpyCas9 | 741 |
| TIGIT4281 | TGGCTCCAAGCAATGGAATC | 20 | SpyCas9 | 742 |
| TIGIT4282 | GCGGCCATGGCTCCAAGCAA | 20 | SpyCas9 | 743 |
| TIGIT4283 | TGGAGCCATGGCCGCGACGC | 20 | SpyCas9 | 744 |
| TIGIT4284 | AGCCATGGCCGCGACGCTGG | 20 | SpyCas9 | 745 |
| TIGIT4285 | GACCACCAGCGTCGCGGCCA | 20 | SpyCas9 | 746 |
| TIGIT4286 | GCAGATGACCACCAGCGTCG | 20 | SpyCas9 | 747 |
| TIGIT4287 | CATCTGCACAGCAGTCATCG | 20 | SpyCas9 | 748 |
| TIGIT4288 | CTGCACAGCAGTCATCGTGG | 20 | SpyCas9 | 749 |
| TIGIT4289 | AGCCCTCAGAATCCATTCTG | 20 | SpyCas9 | 750 |
| TIGIT4290 | CTCAGAATCCATTCTGTGGA | 20 | SpyCas9 | 751 |
| TIGIT4291 | TTCCACAGAATGGATTCTGA | 20 | SpyCas9 | 752 |
| TIGIT4292 | CTTCCACAGAATGGATTCTG | 20 | SpyCas9 | 753 |
| TIGIT4293 | ATTCTGTGGAAGGTGACCTC | 20 | SpyCas9 | 754 |
| TIGIT4294 | TGAGGTCACCTTCCACAGAA | 20 | SpyCas9 | 755 |
| TIGIT4295 | GACCTCAGGAGAAAATCAGC | 20 | SpyCas9 | 756 |
| TIGIT4296 | CAGGAGAAAATCAGCTGGAC | 20 | SpyCas9 | 757 |
| TIGIT4297 | GTCCAGCTGATTTTCTCCTG | 20 | SpyCas9 | 758 |
| TIGIT4298 | GAGAAAATCAGCTGGACAGG | 20 | SpyCas9 | 759 |
| TIGIT4299 | AATCAGCTGGACAGGAGGAA | 20 | SpyCas9 | 760 |
| TIGIT4300 | CCCAGTGCTCCCTCACCCCC | 20 | SpyCas9 | 761 |
| TIGIT4301 | CTGGGGGTGAGGGAGCACTG | 20 | SpyCas9 | 762 |
| TIGIT4302 | CCTGGGGGTGAGGGAGCACT | 20 | SpyCas9 | 763 |
| TIGIT4303 | TCCTGGGGGTGAGGGAGCAC | 20 | SpyCas9 | 764 |
| TIGIT4304 | ACACAGCTTCCTGGGGGTGA | 20 | SpyCas9 | 765 |
| TIGIT4305 | GACACAGCTTCCTGGGGGTG | 20 | SpyCas9 | 766 |
| TIGIT4306 | ACCCCCAGGAAGCTGTGTCC | 20 | SpyCas9 | 767 |
| TIGIT4307 | GCCTGGACACAGCTTCCTGG | 20 | SpyCas9 | 768 |
| TIGIT4308 | TGCCTGGACACAGCTTCCTG | 20 | SpyCas9 | 769 |
| TIGIT4309 | CTGCCTGGACACAGCTTCCT | 20 | SpyCas9 | 770 |
| TIGIT4310 | TCTGCCTGGACACAGCTTCC | 20 | SpyCas9 | 771 |
| TIGIT4311 | CAGGCAGAAGCTGCACCTGC | 20 | SpyCas9 | 772 |
| TIGIT4312 | AGGCAGAAGCTGCACCTGCT | 20 | SpyCas9 | 773 |
| TIGIT4313 | CAGCAGGTGCAGCTTCTGCC | 20 | SpyCas9 | 774 |
| TIGIT4314 | GCTGCACCTGCTGGGCTCTG | 20 | SpyCas9 | 775 |
| TIGIT4315 | TGCTCTCCACAGAGCCCAGC | 20 | SpyCas9 | 776 |
| TIGIT4316 | CTGGGCTCTGTGGAGAGCAG | 20 | SpyCas9 | 777 |
| TIGIT4317 | TGGGCTCTGTGGAGAGCAGC | 20 | SpyCas9 | 778 |
| TIGIT4318 | GGGCTCTGTGGAGAGCAGCG | 20 | SpyCas9 | 779 |
| TIGIT4319 | CTGTGGAGAGCAGCGGGGAG | 20 | SpyCas9 | 780 |
| TIGIT4320 | ATTGAAGTAGTCATGCAGCT | 20 | SpyCas9 | 781 |
| TIGIT4321 | TGTCCTGAGTTACAGAAGCC | 20 | SpyCas9 | 782 |
| TIGIT4322 | GTCCTGAGTTACAGAAGCCT | 20 | SpyCas9 | 783 |
| TIGIT4323 | TACCCAGGCTTCTGTAACTC | 20 | SpyCas9 | 784 |
| TIGIT4324 | TGAAGAAGCTGCAGTTACCC | 20 | SpyCas9 | 785 |
| TIGIT4325 | TGCAGCTTCTTCACAGAGAC | 20 | SpyCas9 | 786 |
| TIGIT5053 | GTTGTTTCTATTGTGCCTGT | 20 | AsCpf1 RR | 787 |
| TIGIT5054 | CGTTGTTTCTATTGTGCCTG | 20 | AsCpf1 RR | 788 |
| TIGIT5055 | CCGTTGTTTCTATTGTGCCT | 20 | AsCpf1 RR | 789 |
| TIGIT5056 | CCACGGCACAAGTGACCCAG | 20 | AsCpf1 RR | 790 |
| TIGIT5057 | AGTTGACCTGGGTCACTTGT | 20 | AsCpf1 RR | 791 |
| TIGIT5058 | AAGTCAGCATTACAAATGGC | 20 | AsCpf1 RR | 792 |
| TIGIT5059 | CATCCTTCAAGGATCGAGTG | 20 | AsCpf1 RR | 793 |
| TIGIT5060 | ATCCTTCAAGGATCGAGTGG | 20 | AsCpf1 RR | 794 |
| TIGIT5061 | AGGATCGAGTGGCCCCAGGT | 20 | AsCpf1 RR | 795 |
| TIGIT5062 | AGGTCCCGGCCTGGGCCTCA | 20 | AsCpf1 RR | 796 |
| TIGIT5063 | GGCCTGGGCCTCACCCTCCA | 20 | AsCpf1 RR | 797 |
| TIGIT5064 | CGGTCAGCGACTGGAGGGTG | 20 | AsCpf1 RR | 798 |
| TIGIT5065 | GTCGCTGACCGTGAACGATA | 20 | AsCpf1 RR | 799 |
| TIGIT5066 | TGTATCGTTCACGGTCAGCG | 20 | AsCpf1 RR | 800 |
| TIGIT5067 | CTGTATCGTTCACGGTCAGC | 20 | AsCpf1 RR | 801 |
| TIGIT5068 | ATCAGGGTAGGTGTGATAGA | 20 | AsCpf1 RR | 802 |
| TIGIT5069 | AGTGTACGTCCCATCAGGGT | 20 | AsCpf1 RR | 803 |
| TIGIT5070 | GGAAGATTCTCCCAGTGTAC | 20 | AsCpf1 RR | 804 |
| TIGIT5071 | TGGAGGTCCTAGAAAGCTCA | 20 | AsCpf1 RR | 805 |
| TIGIT5072 | AGCAATGGAATCTGGAACCT | 20 | AsCpf1 RR | 806 |
| TIGIT5073 | AGATTCCATTGCTTGGAGCC | 20 | AsCpf1 RR | 807 |
| TIGIT5074 | GATTCCATTGCTTGGAGCCA | 20 | AsCpf1 RR | 808 |
| TIGIT5075 | ATTGCTTGGAGCCATGGCCG | 20 | AsCpf1 RR | 809 |
| TIGIT5076 | TTGCTTGGAGCCATGGCCGC | 20 | AsCpf1 RR | 810 |
| TIGIT5077 | CAGAATGGATTCTGAGGGCT | 20 | AsCpf1 RR | 811 |
| TIGIT5078 | ACAGAATGGATTCTGAGGGC | 20 | AsCpf1 RR | 812 |
| TIGIT5079 | TTCTGTGGAAGGTGACCTCA | 20 | AsCpf1 RR | 813 |
| TIGIT5080 | GCTGATTTTCTCCTGAGGTC | 20 | AsCpf1 RR | 814 |
| TIGIT5081 | TCCTGTCCAGCTGATTTTCT | 20 | AsCpf1 RR | 815 |
| TIGIT5082 | TTCCTCCTGTCCAGCTGATT | 20 | AsCpf1 RR | 816 |
| TIGIT5083 | TGGGGGTGAGGGAGCACTGG | 20 | AsCpf1 RR | 817 |
| TIGIT5084 | AGTGCTCCCTCACCCCCAGG | 20 | AsCpf1 RR | 818 |
| TIGIT5085 | TCACCCCCAGGAAGCTGTGT | 20 | AsCpf1 RR | 819 |
| TIGIT5086 | CAGGAAGCTGTGTCCAGGCA | 20 | AsCpf1 RR | 820 |
| TIGIT5087 | AGGAAGCTGTGTCCAGGCAG | 20 | AsCpf1 RR | 821 |
| TIGIT5088 | GGCAGAAGCTGCACCTGCTG | 20 | AsCpf1 RR | 822 |
| TIGIT5089 | CAGAGCCCAGCAGGTGCAGC | 20 | AsCpf1 RR | 823 |
| TIGIT5090 | GCTGCTCTCCACAGAGCCCA | 20 | AsCpf1 RR | 824 |
| TIGIT5091 | CGCTGCTCTCCACAGAGCCC | 20 | AsCpf1 RR | 825 |
| TIGIT5092 | ATGTCCTGAGTTACAGAAGC | 20 | AsCpf1 RR | 826 |
| TIGIT5093 | TGCAGAGAAAGGTGGCTCTAT | 21 | Cas12a | 1175 |

In some embodiments the gRNA for use in the disclosure is a gRNA targeting ADORA2a (ADORA2a gRNA). In some embodiments, the gRNA targeting ADORA2a is one or more of the gRNAs described in Table 12.

TABLE 12
Exemplary ADORA2a gRNAs

| | gRNA Targeting Domain | | | SEQ ID |
|---|---|---|---|---|
| Name | Sequence (DNA) | Length | Enzyme | NO: |
| ADORA2A337 | GAGCACACCCACTGCGATGT | 20 | SpyCas9 | 827 |
| ADORA2A338 | GATGGCCAGGAGACTGAAGA | 20 | SpyCas9 | 828 |
| ADORA2A339 | CTGCTCACCGGAGCGGGATG | 20 | SpyCas9 | 829 |
| ADORA2A340 | GTCTGTGGCCATGCCCATCA | 20 | SpyCas9 | 830 |
| ADORA2A341 | TCACCGGAGCGGGATGCGGA | 20 | SpyCas9 | 831 |
| ADORA2A342 | GTGGCAGGCAGCGCAGAACC | 20 | SpyCas9 | 832 |
| ADORA2A343 | AGCACACCAGCACATTGCCC | 20 | SpyCas9 | 833 |
| ADORA2A344 | CAGGTTGCTGTTGAGCCACA | 20 | SpyCas9 | 834 |
| ADORA2A345 | CTTCATTGCCTGCTTCGTCC | 20 | SpyCas9 | 835 |
| ADORA2A346 | GTACACCGAGGAGCCCATGA | 20 | SpyCas9 | 836 |
| ADORA2A347 | GATGGCAATGTAGCGGTCAA | 20 | SpyCas9 | 837 |
| ADORA2A348 | CTCCTCGGTGTACATCACGG | 20 | SpyCas9 | 838 |
| ADORA2A349 | CGAGGAGCCCATGATGGGCA | 20 | SpyCas9 | 839 |
| ADORA2A350 | GGGCTCCTCGGTGTACATCA | 20 | SpyCas9 | 840 |
| ADORA2A351 | CTTTGTGGTGTCACTGGCGG | 20 | SpyCas9 | 841 |
| ADORA2A352 | CCGCTCCGGTGAGCAGGGCC | 20 | SpyCas9 | 842 |
| ADORA2A353 | GGGTTCTGCGCTGCCTGCCA | 20 | SpyCas9 | 843 |
| ADORA2A354 | GGACGAAGCAGGCAATGAAG | 20 | SpyCas9 | 844 |
| ADORA2A355 | GTGCTGATGGTGATGGCAAA | 20 | SpyCas9 | 845 |
| ADORA2A356 | AGCGCAGAACCCGGTGCTGA | 20 | SpyCas9 | 846 |
| ADORA2A357 | GAGCTCCATCTTCAGTCTCC | 20 | SpyCas9 | 847 |
| ADORA2A358 | TGCTGATGGTGATGGCAAAG | 20 | SpyCas9 | 848 |
| ADORA2A359 | GGCGGCGGCCGACATCGCAG | 20 | SpyCas9 | 849 |
| ADORA2A360 | AATGAAGAGGCAGCCGTGGC | 20 | SpyCas9 | 850 |
| ADORA2A361 | GGGCAATGTGCTGGTGTGCT | 20 | SpyCas9 | 851 |
| ADORA2A362 | CATGCCCATCATGGGCTCCT | 20 | SpyCas9 | 852 |
| ADORA2A363 | AATGTAGCGGTCAATGGCGA | 20 | SpyCas9 | 853 |
| ADORA2A364 | AGTAGTTGGTGACGTTCTGC | 20 | SpyCas9 | 854 |
| ADORA2A365 | AGCGGTCAATGGCGATGGCC | 20 | SpyCas9 | 855 |
| ADORA2A366 | CGCATCCCGCTCCGGTGAGC | 20 | SpyCas9 | 856 |
| ADORA2A367 | GCATCCCGCTCCGGTGAGCA | 20 | SpyCas9 | 857 |
| ADORA2A368 | TGGGCAATGTGCTGGTGTGC | 20 | SpyCas9 | 858 |
| ADORA2A369 | CAACTACTTTGTGGTGTCAC | 20 | SpyCas9 | 859 |
| ADORA2A370 | CGCTCCGGTGAGCAGGGCCG | 20 | SpyCas9 | 860 |
| ADORA2A371 | GATGGTGATGGCAAAGGGGA | 20 | SpyCas9 | 861 |
| ADORA2A372 | GGTGTACATCACGGTGGAGC | 20 | SpyCas9 | 862 |
| ADORA2A373 | GAACGTCACCAACTACTTTG | 20 | SpyCas9 | 863 |
| ADORA2A374 | CAGTGACACCACAAAGTAGT | 20 | SpyCas9 | 864 |
| ADORA2A375 | GGCCATCCTGGGCAATGTGC | 20 | SpyCas9 | 865 |
| ADORA2A376 | CCCGGCCCTGCTCACCGGAG | 20 | SpyCas9 | 866 |
| ADORA2A377 | CACCAGCACATTGCCCAGGA | 20 | SpyCas9 | 867 |
| ADORA2A378 | TTTGCCATCACCATCAGCAC | 20 | SpyCas9 | 868 |
| ADORA2A379 | CTCCACCGTGATGTACACCG | 20 | SpyCas9 | 869 |
| ADORA2A380 | GGAGCTGGCCATTGCTGTGC | 20 | SpyCas9 | 870 |
| ADORA2A381 | CAGGATGGCCAGCACAGCAA | 20 | SpyCas9 | 871 |
| ADORA2A382 | GAACCCGGTGCTGATGGTGA | 20 | SpyCas9 | 872 |
| ADORA2A383 | TGGAGCTCTGCGTGAGGACC | 20 | SpyCas9 | 873 |
| ADORA2A384 | CCCGCTCCGGTGAGCAGGGC | 20 | SpyCas9 | 874 |
| ADORA2A385 | AGGCAATGAAGAGGCAGCCG | 20 | SpyCas9 | 875 |
| ADORA2A386 | CCGGCCCTGCTCACCGGAGC | 20 | SpyCas9 | 876 |
| ADORA2A387 | GCGGCGGCCGACATCGCAGT | 20 | SpyCas9 | 877 |
| ADORA2A388 | GGTGCTGATGGTGATGGCAA | 20 | SpyCas9 | 878 |
| ADORA2A389 | CTACTTTGTGGTGTCACTGG | 20 | SpyCas9 | 879 |
| ADORA2A390 | TACACCGAGGAGCCCATGAT | 20 | SpyCas9 | 880 |
| ADORA2A391 | TCTGTGGCCATGCCCATCAT | 20 | SpyCas9 | 881 |
| ADORA2A392 | ATTGCTGTGCTGGCCATCCT | 20 | SpyCas9 | 882 |
| ADORA2A393 | CGTGAGGACCAGGACGAAGC | 20 | SpyCas9 | 883 |
| ADORA2A394 | TTGCCATCACCATCAGCACC | 20 | SpyCas9 | 884 |
| ADORA2A395 | GGATGCGGATGGCAATGTAG | 20 | SpyCas9 | 885 |
| ADORA2A396 | TTGCCATCCGCATCCCGCTC | 20 | SpyCas9 | 886 |
| ADORA2A397 | TGAAGATGGAGCTCTGCGTG | 20 | SpyCas9 | 887 |
| ADORA2A398 | CATTGCTGTGCTGGCCATCC | 20 | SpyCas9 | 888 |
| ADORA2A399 | TGCTGGTGTGCTGGGCCGTG | 20 | SpyCas9 | 889 |
| ADORA2A820 | GGCTCCTCGGTGTACATCACG | 21 | SauCas9 | 890 |
| ADORA2A821 | GAGCTCTGCGTGAGGACCAGG | 21 | SauCas9 | 891 |
| ADORA2A822 | GATGGAGCTCTGCGTGAGGAC | 21 | SauCas9 | 892 |
| ADORA2A823 | CCAGCACACCAGCACATTGCC | 21 | SauCas9 | 893 |
| ADORA2A824 | AGGACCAGGACGAAGCAGGCA | 21 | SauCas9 | 894 |
| ADORA2A825 | TGCCATCCGCATCCCGCTCCG | 21 | SauCas9 | 895 |
| ADORA2A826 | GTGTGGCTCAACAGCAACCTG | 21 | SauCas9 | 896 |
| ADORA2A827 | AGCTCCACCGTGATGTACACC | 21 | SauCas9 | 897 |
| ADORA2A828 | GTAGCGGTCAATGGCGATGGC | 21 | SauCas9 | 898 |
| ADORA2A829 | CGGTGCTGATGGTGATGGCAA | 21 | SauCas9 | 899 |
| ADORA2A830 | CCCTGCTCACCGGAGCGGGAT | 21 | SauCas9 | 900 |
| ADORA2A831 | GTGACGTTCTGCAGGTTGCTG | 21 | SauCas9 | 901 |
| ADORA2A832 | GCTCCACCGTGATGTACACCG | 21 | SauCas9 | 902 |
| ADORA2A833 | ACTGAAGATGGAGCTCTGCGT | 21 | SauCas9 | 903 |
| ADORA2A834 | CCAGCTCCACCGTGATGTACA | 21 | SauCas9 | 904 |
| ADORA2A835 | CCTTTGCCATCACCATCAGCA | 21 | SauCas9 | 905 |
| ADORA2A836 | CCGGTGCTGATGGTGATGGCA | 21 | SauCas9 | 906 |
| ADORA2A837 | CCTGGGCAATGTGCTGGTGTG | 21 | SauCas9 | 907 |
| ADORA2A838 | AGGCAGCCGTGGCAGGCAGCG | 21 | SauCas9 | 908 |
| ADORA2A839 | GCGATGGCCAGGAGACTGAAG | 21 | SauCas9 | 909 |
| ADORA2A840 | CGATGGCCAGGAGACTGAAGA | 21 | SauCas9 | 910 |
| ADORA2A841 | TCCCGCTCCGGTGAGCAGGGC | 21 | SauCas9 | 911 |
| ADORA2A842 | TGCTTCGTCCTGGTCCTCACG | 21 | SauCas9 | 912 |
| ADORA2A843 | ACCAGGACGAAGCAGGCAATG | 21 | SauCas9 | 913 |
| ADORA2A844 | ATGTACACCGAGGAGCCCATG | 21 | SauCas9 | 914 |
| ADORA2A845 | TCGTCTGTGGCCATGCCCATC | 21 | SauCas9 | 915 |
| ADORA2A846 | TCAATGGCGATGGCCAGGAGA | 21 | SauCas9 | 916 |
| ADORA2A847 | GGTGCTGATGGTGATGGCAAA | 21 | SauCas9 | 917 |
| ADORA2A848 | TAGCGGTCAATGGCGATGGCC | 21 | SauCas9 | 918 |
| ADORA2A849 | TCCGCATCCCGCTCCGGTGAG | 21 | SauCas9 | 919 |
| ADORA2A850 | CTGGCGGCGGCCGACATCGCA | 21 | SauCas9 | 920 |
| ADORA2A851 | GCCATTGCTGTGCTGGCCATC | 21 | SauCas9 | 921 |
| ADORA2A852 | ATCCCGCTCCGGTGAGCAGGG | 21 | SauCas9 | 922 |
| ADORA2A853 | AGACTGAAGATGGAGCTCTGC | 21 | SauCas9 | 923 |
| ADORA2A854 | CCCCGGCCCTGCTCACCGGAG | 21 | SauCas9 | 924 |
| ADORA2A855 | ATGGTGATGGCAAAGGGGATG | 21 | SauCas9 | 925 |
| ADORA2A856 | GCTCCTCGGTGTACATCACGG | 21 | SauCas9 | 926 |
| ADORA2A248 | TGTCGATGGCAATAGCCAAG | 20 | SpyCas9 | 927 |
| ADORA2A249 | AGAAGTTGGTGACGTTCTGC | 20 | SpyCas9 | 928 |
| ADORA2A250 | TTCGCCATCACCATCAGCAC | 20 | SpyCas9 | 929 |
| ADORA2A251 | GAAGAAGAGGCAGCCATGGC | 20 | SpyCas9 | 930 |
| ADORA2A252 | CACAAGCACGTTACCCAGGA | 20 | SpyCas9 | 931 |
| ADORA2A253 | CAACTTCTTCGTGGTATCTC | 20 | SpyCas9 | 932 |
| ADORA2A254 | CAGGATGGCCAGCACAGCAA | 20 | SpyCas9 | 933 |
| ADORA2A255 | AATTCCACTCCGGTGAGCCA | 20 | SpyCas9 | 934 |
| ADORA2A256 | AGCGCAGAAGCCAGTGCTGA | 20 | SpyCas9 | 935 |
| ADORA2A257 | GTGCTGATGGTGATGGCGAA | 20 | SpyCas9 | 936 |
| ADORA2A258 | GGAGCTGGCCATTGCTGTGC | 20 | SpyCas9 | 937 |
| ADORA2A259 | AATAGCCAAGAGGCTGAAGA | 20 | SpyCas9 | 938 |
| ADORA2A260 | CTCCTCGGTGTACATCATGG | 20 | SpyCas9 | 939 |
| ADORA2A261 | GGACAAAGCAGGCGAAGAAG | 20 | SpyCas9 | 940 |
| ADORA2A262 | TCTGGCGGCGGCTGACATCG | 20 | SpyCas9 | 941 |
| ADORA2A263 | TGGGTAACGTGCTTGTGTGC | 20 | SpyCas9 | 942 |
| ADORA2A264 | GATGTACACCGAGGAGCCCA | 20 | SpyCas9 | 943 |
| ADORA2A265 | TAACCCCTGGCTCACCGGAG | 20 | SpyCas9 | 944 |
| ADORA2A266 | TCACCGGAGTGGAATTCGGA | 20 | SpyCas9 | 945 |
| ADORA2A267 | GCGGCGGCTGACATCGCGGT | 20 | SpyCas9 | 946 |
| ADORA2A268 | GATGGTGATGGCGAATGGGA | 20 | SpyCas9 | 947 |
| ADORA2A269 | GGCTTCTGCGCTGCCTGCCA | 20 | SpyCas9 | 948 |
| ADORA2A270 | ATTCCACTCCGGTGAGCCAG | 20 | SpyCas9 | 949 |
| ADORA2A271 | GGTGTACATCATGGTGGAGC | 20 | SpyCas9 | 950 |
| ADORA2A272 | ATTGCTGTGCTGGCCATCCT | 20 | SpyCas9 | 95 |
| ADORA2A273 | CTCCACCATGATGTACACCG | 20 | SpyCas9 | 952 |
| ADORA2A274 | GGCGGCGGCTGACATCGCGG | 20 | SpyCas9 | 953 |
| ADORA2A275 | TACACCGAGGAGCCCATGGC | 20 | SpyCas9 | 954 |
| ADORA2A276 | GGGTAACGTGCTTGTGTGCT | 20 | SpyCas9 | 955 |
| ADORA2A277 | CAGGTTGCTGTTGATCCACA | 20 | SpyCas9 | 956 |
| ADORA2A278 | TGAAGATGGAACTCTGCGTG | 20 | SpyCas9 | 957 |
| ADORA2A279 | GATGGCGATGTATCTGTCGA | 20 | SpyCas9 | 958 |
| ADORA2A280 | CTTCTTCGCCTGCTTTGTCC | 20 | SpyCas9 | 959 |
| ADORA2A281 | AGGCGAAGAAGAGGCAGCCA | 20 | SpyCas9 | 960 |
| ADORA2A282 | TGCTTGTGTGCTGGGCCGTG | 20 | SpyCas9 | 961 |
| ADORA2A283 | GAAGCCAGTGCTGATGGTGA | 20 | SpyCas9 | 962 |
| ADORA2A284 | CGTGAGGACCAGGACAAAGC | 20 | SpyCas9 | 963 |
| ADORA2A285 | TGGAACTCTGCGTGAGGACC | 20 | SpyCas9 | 964 |
| ADORA2A286 | CATTGCTGTGCTGGCCATCC | 20 | SpyCas9 | 965 |
| ADORA2A287 | TTCTCCCGCCATGGGCTCCT | 20 | SpyCas9 | 966 |
| ADORA2A288 | TGGCTCACCGGAGTGGAATT | 20 | SpyCas9 | 967 |
| ADORA2A289 | TGCTGATGGTGATGGCGAAT | 20 | SpyCas9 | 968 |
| ADORA2A290 | CTTCGTGGTATCTCTGGCGG | 20 | SpyCas9 | 969 |
| ADORA2A291 | AGCACACAAGCACGTTACCC | 20 | SpyCas9 | 970 |
| ADORA2A292 | GGGCTCCTCGGTGTACATCA | 20 | SpyCas9 | 971 |
| ADORA2A293 | GTACACCGAGGAGCCCATGG | 20 | SpyCas9 | 972 |
| ADORA2A294 | GAACGTCACCAACTTCTTCG | 20 | SpyCas9 | 973 |
| ADORA2A295 | TCGCCATCCGAATTCCACTC | 20 | SpyCas9 | 974 |
| ADORA2A296 | GAGTTCCATCTTCAGCCTCT | 20 | SpyCas9 | 975 |
| ADORA2A297 | GAATTCCACTCCGGTGAGCC | 20 | SpyCas9 | 976 |
| ADORA2A298 | CAGAGATACCACGAAGAAGT | 20 | SpyCas9 | 977 |
| ADORA2A299 | CTTCTTCGTGGTATCTCTGG | 20 | SpyCas9 | 978 |
| ADORA2A695 | CAGTGCTGATGGTGATGGCGA | 21 | SauCas9 | 979 |
| ADORA2A696 | CGAATTCCACTCCGGTGAGCC | 21 | SauCas9 | 980 |
| ADORA2A697 | CCGAATTCCACTCCGGTGAGC | 21 | SauCas9 | 981 |
| ADORA2A698 | GCTGAAGATGGAACTCTGCGT | 21 | SauCas9 | 982 |
| ADORA2A699 | CGTGCTTGTGTGCTGGGCCGT | 21 | SauCas9 | 983 |
| ADORA2A700 | GTGAGGACCAGGACAAAGCAG | 21 | SauCas9 | 984 |
| ADORA2A701 | TCGATGGCAATAGCCAAGAGG | 21 | SauCas9 | 985 |
| ADORA2A702 | CATCGACAGATACATCGCCAT | 21 | SauCas9 | 986 |
| ADORA2A703 | GTACACCGAGGAGCCCATGGC | 21 | SauCas9 | 987 |
| ADORA2A704 | GCTCCACCATGATGTACACCG | 21 | SauCas9 | 988 |
| ADORA2A705 | AAGCCAGTGCTGATGGTGATG | 21 | SauCas9 | 989 |
| ADORA2A706 | CACCGCGATGTCAGCCGCCGC | 21 | SauCas9 | 990 |
| ADORA2A707 | AGGCTGAAGATGGAACTCTGC | 21 | SauCas9 | 991 |
| ADORA2A708 | GCCGCCGCCAGAGATACCACG | 21 | SauCas9 | 992 |
| ADORA2A709 | AGCTCCACCATGATGTACACC | 21 | SauCas9 | 993 |
| ADORA2A710 | AGGCAGCCATGGCAGGCAGCG | 21 | SauCas9 | 994 |
| ADORA2A711 | CCTGGCTCACCGGAGTGGAAT | 21 | SauCas9 | 995 |
| ADORA2A712 | CCAGCTCCACCATGATGTACA | 21 | SauCas9 | 996 |
| ADORA2A713 | ACCAGGACAAAGCAGGCGAAG | 21 | SauCas9 | 997 |
| ADORA2A714 | CCTGGGTAACGTGCTTGTGTG | 21 | SauCas9 | 998 |
| ADORA2A715 | AGGACCAGGACAAAGCAGGCG | 21 | SauCas9 | 999 |
| ADORA2A716 | TCAGCCGCCGCCAGAGATACC | 21 | SauCas9 | 1000 |
| ADORA2A717 | GGCTCCTCGGTGTACATCATG | 21 | SauCas9 | 1001 |
| ADORA2A718 | CTGGCGGCGGCTGACATCGCG | 21 | SauCas9 | 1002 |
| ADORA2A719 | GATGGAACTCTGCGTGAGGAC | 21 | SauCas9 | 1003 |
| ADORA2A720 | GCTCCTCGGTGTACATCATGG | 21 | SauCas9 | 1004 |
| ADORA2A721 | TGTACACCGAGGAGCCCATGG | 21 | SauCas9 | 1005 |
| ADORA2A722 | GCCATTGCTGTGCTGGCCATC | 21 | SauCas9 | 1006 |
| ADORA2A723 | CAATAGCCAAGAGGCTGAAGA | 21 | SauCas9 | 1007 |
| ADORA2A724 | ATGGTGATGGCGAATGGGATG | 21 | SauCas9 | 1008 |
| ADORA2A725 | ATGTACACCGAGGAGCCCATG | 21 | SauCas9 | 1009 |
| ADORA2A726 | GTGTGGATCAACAGCAACCTG | 21 | SauCas9 | 1010 |
| ADORA2A727 | TGCTTTGTCCTGGTCCTCACG | 21 | SauCas9 | 1011 |
| ADORA2A728 | GTAACCCCTGGCTCACCGGAG | 21 | SauCas9 | 1012 |
| ADORA2A729 | CCAGCACACAAGCACGTTACC | 21 | SauCas9 | 1013 |
| ADORA2A730 | TATCTGTCGATGGCAATAGCC | 21 | SauCas9 | 1014 |
| ADORA2A731 | GCAATAGCCAAGAGGCTGAAG | 21 | SauCas9 | 1015 |
| ADORA2A732 | AGTGCTGATGGTGATGGCGAA | 21 | SauCas9 | 1016 |
| ADORA2A733 | ACACCGAGGAGCCCATGGCGG | 21 | SauCas9 | 1017 |
| ADORA2A734 | CGCCATCCGAATTCCACTCCG | 21 | SauCas9 | 1018 |
| ADORA2A4111 | TGGTGTCACTGGCGGCGGCC | 20 | AsCpf1 | 1019 |
| ADORA2A4112 | CCATCACCATCAGCACCGGG | 20 | AsCpf1 | 1020 |
| ADORA2A4113 | CCATCGGCCTGACTCCCATG | 20 | AsCpf1 | 1021 |
| ADORA2A4114 | GCTGACCGCAGTTGTTCCAA | 20 | AsCpf1 | 1022 |
| ADORA2A4115 | AGGATGTGGTCCCCATGAAC | 20 | AsCpf1 | 1023 |
| ADORA2A4116 | CCTGTGTGCTGGTGCCCCTG | 20 | AsCpf1 | 1024 |
| ADORA2A4117 | CGGATCTTCCTGGCGGCGCG | 20 | AsCpf1 | 1025 |
| ADORA2A4118 | CCCTCTGCTGGCTGCCCCTA | 20 | AsCpf1 | 1026 |
| ADORA2A4119 | TTCTGCCCCGACTGCAGCCA | 20 | AsCpf1 | 1027 |
| ADORA2A4120 | AAGGCAGCTGGCACCAGTGC | 20 | AsCpf1 | 1028 |
| ADORA2A4121 | TAAGGGCATCATTGCCATCTG | 21 | SauCas9 | 1029 |
| ADORA2A4122 | CGGCCTGACTCCCATGCTAGG | 21 | SauCas9 | 1030 |
| ADORA2A4123 | GCAGTTGTTCCAACCTAGCAT | 21 | SauCas9 | 1031 |
| ADORA2A4124 | CCGCAGTTGTTCCAACCTAGC | 21 | SauCas9 | 1032 |
| ADORA2A4125 | CAAGAACCACTCCCAGGGCTG | 21 | SauCas9 | 1033 |
| ADORA2A4126 | CTTGGCCCTCCCCGCAGCCCT | 21 | SauCas9 | 1034 |
| ADORA2A4127 | CACTTGGCCCTCCCCGCAGCC | 21 | SauCas9 | 1035 |
| ADORA2A4128 | GGCCAAGTGGCCTGTCTCTTT | 21 | SauCas9 | 1036 |
| ADORA2A4129 | TTCATGGGGACCACATCCTCA | 21 | SauCas9 | 1037 |
| ADORA2A4130 | TGAAGTACACCATGTAGTTCA | 21 | SauCas9 | 1038 |
| ADORA2A4131 | CTGGTGCCCCTGCTGCTCATG | 21 | SauCas9 | 1039 |
| ADORA2A4132 | GCTCATGCTGGGTGTCTATTT | 21 | SauCas9 | 1040 |
| ADORA2A4133 | CTTCAGCTGTCGTCGCGCCGC | 21 | SauCas9 | 1041 |
| ADORA2A4134 | CGCGACGACAGCTGAAGCAGA | 21 | SauCas9 | 1042 |
| ADORA2A4135 | GATGGAGAGCCAGCCTCTGCC | 21 | SauCas9 | 1043 |
| ADORA2A4136 | GCGTGGCTGCAGTCGGGGCAG | 21 | SauCas9 | 1044 |
| ADORA2A4137 | ACGATGGCCAGGTACATGAGC | 21 | SauCas9 | 1045 |
| ADORA2A4138 | CTCTCCCACACCAATTCGGTT | 21 | SauCas9 | 1046 |
| ADORA2A4139 | GATTCACAACCGAATTGGTGT | 21 | SauCas9 | 1047 |
| ADORA2A4140 | GGGATTCACAACCGAATTGGT | 21 | SauCas9 | 1048 |
| ADORA2A4141 | CGTAGATGAAGGGATTCACAA | 21 | SauCas9 | 1049 |
| ADORA2A4142 | GGATACGGTAGGCGTAGATGA | 21 | SauCas9 | 1050 |
| ADORA2A4143 | TCATCTACGCCTACCGTATCC | 21 | SauCas9 | 1051 |
| ADORA2A4144 | CGGATACGGTAGGCGTAGATG | 21 | SauCas9 | 1052 |
| ADORA2A4145 | GCGGAAGGTCTGGCGGAACTC | 21 | SauCas9 | 1053 |
| ADORA2A4146 | AATGATCTTGCGGAAGGTCTG | 21 | SauCas9 | 1054 |
| ADORA2A4147 | GACGTGGCTGCGAATGATCTT | 21 | SauCas9 | 1055 |
| ADORA2A4148 | TTGCTGCCTCAGGACGTGGCT | 21 | SauCas9 | 1056 |
| ADORA2A4149 | CAAGGCAGCTGGCACCAGTGC | 21 | SauCas9 | 1057 |
| ADORA2A4150 | CGGGCACTGGTGCCAGCTGCC | 21 | SauCas9 | 1058 |
| ADORA2A4151 | CTTGGCAGCTCATGGCAGTGA | 21 | SauCas9 | 1059 |
| ADORA2A4152 | CCGTCTCAACGGCCACCCGCC | 21 | SauCas9 | 1060 |
| ADORA2A4153 | CACACTCCTGGCGGGTGGCCG | 21 | SauCas9 | 1061 |
| ADORA2A4154 | TGCCGTTGGCCCACACTCCTG | 21 | SauCas9 | 1062 |
| ADORA2A4155 | CCATTGGGCCTCCGCTCAGGG | 21 | SauCas9 | 1063 |
| ADORA2A4156 | CATAGCCATTGGGCCTCCGCT | 21 | SauCas9 | 1064 |
| ADORA2A4157 | AATGGCTATGCCCTGGGGCTG | 21 | SauCas9 | 1065 |
| ADORA2A4158 | ATGCCCTGGGGCTGGTGAGTG | 21 | SauCas9 | 1066 |
| ADORA2A4159 | GCCCTGGGGCTGGTGAGTGGA | 21 | SauCas9 | 1067 |
| ADORA2A4160 | TGGTGAGTGGAGGGAGTGCCC | 21 | SauCas9 | 1068 |
| ADORA2A4161 | GAGGGAGTGCCCAAGAGTCCC | 21 | SauCas9 | 1069 |
| ADORA2A4162 | AGGGAGTGCCCAAGAGTCCCA | 21 | SauCas9 | 1070 |
| ADORA2A4163 | GTCTGGGAGGCCCGTGTTCCC | 21 | SauCas9 | 1071 |
| ADORA2A4164 | CATGGCTAAGGAGCTCCACGT | 21 | SauCas9 | 1072 |
| ADORA2A4165 | GAGCTCCTTAGCCATGAGCTC | 21 | SauCas9 | 1073 |
| ADORA2A4166 | GCTCCTTAGCCATGAGCTCAA | 21 | SauCas9 | 1074 |
| ADORA2A4167 | GGCCTAGATGACCCCCTGGCC | 21 | SauCas9 | 1075 |
| ADORA2A4168 | CCCCCTGGCCCAGGATGGAGC | 21 | SauCas9 | 1076 |
| ADORA2A4169 | CTCCTGCTCCATCCTGGGCCA | 21 | SauCas9 | 1077 |
| ADORA2A4416 | CCGTGATGTACACCGAGGAG | 20 | AsCpf1 RR | 1078 |
| ADORA2A4417 | CTTTGCCATCACCATCAGCA | 20 | AsCpf1 RR | 1079 |
| ADORA2A4418 | TTTGCCATCACCATCAGCAC | 20 | AsCpf1 RR | 1080 |
| ADORA2A4419 | TTGCCTGCTTCGTCCTGGTC | 20 | AsCpf1 RR | 1081 |
| ADORA2A4420 | TCCTGGTCCTCACGCAGAGC | 20 | AsCpf1 RR | 1082 |
| ADORA2A4421 | TCTTCAGTCTCCTGGCCATC | 20 | AsCpf1 RR | 1083 |
| ADORA2A4422 | GTCTCCTGGCCATCGCCATT | 20 | AsCpf1 RR | 1084 |
| ADORA2A4423 | ACCTAGCATGGGAGTCAGGC | 20 | AsCpf1 RR | 1085 |
| ADORA2A4424 | AACCTAGCATGGGAGTCAGG | 20 | AsCpf1 RR | 1086 |
| ADORA2A4425 | ATGCTAGGTTGGAACAACTG | 20 | AsCpf1 RR | 1087 |
| ADORA2A4426 | GCAGCCCTGGGAGTGGTTCT | 20 | AsCpf1 RR | 1088 |
| ADORA2A4427 | CGCAGCCCTGGGAGTGGTTC | 20 | AsCpf1 RR | 1089 |
| ADORA2A4428 | AGGGCTGCGGGGAGGGCCAA | 20 | AsCpf1 RR | 1090 |
| ADORA2A4429 | TGGGGACCACATCCTCAAAG | 20 | AsCpf1 RR | 1091 |
| ADORA2A4430 | CATGAACTACATGGTGTACT | 20 | AsCpf1 RR | 1092 |
| ADORA2A4431 | ATGAACTACATGGTGTACTT | 20 | AsCpf1 RR | 1093 |
| ADORA2A4432 | ACTTCTTTGCCTGTGTGCTG | 20 | AsCpf1 RR | 1094 |
| ADORA2A4433 | TGCTGCTCATGCTGGGTGTC | 20 | AsCpf1 RR | 1095 |
| ADORA2A4434 | CAAATAGACACCCAGCATGA | 20 | AsCpf1 RR | 1096 |
| ADORA2A4435 | GCTGTCGTCGCGCCGCCAGG | 20 | AsCpf1 RR | 1097 |
| ADORA2A4436 | TGGCGGCGCGACGACAGCTG | 20 | AsCpf1 RR | 1098 |
| ADORA2A4437 | TCTGCTTCAGCTGTCGTCGC | 20 | AsCpf1 RR | 1099 |
| ADORA2A4438 | GGCAGAGGCTGGCTCTCCAT | 20 | AsCpf1 RR | 1100 |
| ADORA2A4439 | CGGCAGAGGCTGGCTCTCCA | 20 | AsCpf1 RR | 1101 |
| ADORA2A4440 | CCGGCAGAGGCTGGCTCTCC | 20 | AsCpf1 RR | 1102 |
| ADORA2A4441 | CACTGCAGAAGGAGGTCCAT | 20 | AsCpf1 RR | 1103 |
| ADORA2A4442 | TGCTGCCAAGTCACTGGCCA | 20 | AsCpf1 RR | 1104 |
| ADORA2A4443 | ACAATGATGGCCAGTGACTT | 20 | AsCpf1 RR | 1105 |
| ADORA2A4444 | TACACATCATCAACTGCTTC | 20 | AsCpf1 RR | 1106 |
| ADORA2A4445 | CTTTCTTCTGCCCCGACTGC | 20 | AsCpf1 RR | 1107 |
| ADORA2A4446 | GACTGCAGCCACGCCCCTCT | 20 | AsCpf1 RR | 1108 |
| ADORA2A4447 | TCTCTGGCTCATGTACCTGG | 20 | AsCpf1 RR | 1109 |
| ADORA2A4448 | CAACCGAATTGGTGTGGGAG | 20 | AsCpf1 RR | 1110 |
| ADORA2A4449 | ACACCAATTCGGTTGTGAAT | 20 | AsCpf1 RR | 1111 |
| ADORA2A4450 | GTTGTGAATCCCTTCATCTA | 20 | AsCpf1 RR | 1112 |
| ADORA2A4451 | TTCATCTACGCCTACCGTAT | 20 | AsCpf1 RR | 1113 |
| ADORA2A4452 | TCTACGCCTACCGTATCCGC | 20 | AsCpf1 RR | 1114 |
| ADORA2A4453 | CGAGTTCCGCCAGACCTTCC | 20 | AsCpf1 RR | 1115 |
| ADORA2A4454 | GCCAGACCTTCCGCAAGATC | 20 | AsCpf1 RR | 1116 |
| ADORA2A4455 | CCAGACCTTCCGCAAGATCA | 20 | AsCpf1 RR | 1117 |
| ADORA2A4456 | GCAAGATCATTCGCAGCCAC | 20 | AsCpf1 RR | 1118 |
| ADORA2A4457 | CAAGATCATTCGCAGCCACG | 20 | AsCpf1 RR | 1119 |
| ADORA2A4458 | CAGCCACGTCCTGAGGCAGC | 20 | AsCpf1 RR | 1120 |
| ADORA2A4459 | AGGCAGCTGGCACCAGTGCC | 20 | AsCpf1 RR | 1121 |
| ADORA2A4460 | TCACTGCCATGAGCTGCCAA | 20 | AsCpf1 RR | 1122 |
| ADORA2A4461 | TCTCAACGGCCACCCGCCAG | 20 | AsCpf1 RR | 1123 |
| ADORA2A4462 | CTCAGGGTGGGGAGCACTGC | 20 | AsCpf1 RR | 1124 |
| ADORA2A4463 | CACCCTGAGCGGAGGCCCAA | 20 | AsCpf1 RR | 1125 |
| ADORA2A4464 | ACCCTGAGCGGAGGCCCAAT | 20 | AsCpf1 RR | 1126 |
| ADORA2A4465 | AGGGCATAGCCATTGGGCCT | 20 | AsCpf1 RR | 1127 |
| ADORA2A4466 | CTCACCAGCCCCAGGGCATA | 20 | AsCpf1 RR | 1128 |
| ADORA2A4467 | TCCACTCACCAGCCCCAGGG | 20 | AsCpf1 RR | 1129 |
| ADORA2A4468 | TGGGACTCTTGGGCACTCCC | 20 | AsCpf1 RR | 1130 |
| ADORA2A4469 | CTGGGACTCTTGGGCACTCC | 20 | AsCpf1 RR | 1131 |
| ADORA2A4470 | CCTGGGACTCTTGGGCACTC | 20 | AsCpf1 RR | 1132 |
| ADORA2A4471 | AGGGGAACACGGGCCTCCCA | 20 | AsCpf1 RR | 1133 |
| ADORA2A4472 | CGTCTGGGAGGCCCGTGTTC | 20 | AsCpf1 RR | 1134 |
| ADORA2A4473 | AGACGTGGAGCTCCTTAGCC | 20 | AsCpf1 RR | 1135 |
| ADORA2A4474 | TTGAGCTCATGGCTAAGGAG | 20 | AsCpf1 RR | 1136 |
| ADORA2A4475 | CTGGCCTAGATGACCCCCTG | 20 | AsCpf1 RR | 1137 |
| ADORA2A4476 | TGGCCTAGATGACCCCCTGG | 20 | AsCpf1 RR | 1138 |
| ADORA2A4477 | TCCTGGGCCAGGGGGTCATC | 20 | AsCpf1 RR | 1139 |
| ADORA2A4478 | CTGGCCCAGGATGGAGCAGG | 20 | AsCpf1 RR | 1140 |
| ADORA2A4479 | TGGCCCAGGATGGAGCAGGA | 20 | AsCpf1 RR | 1141 |
| ADORA2A4480 | CGCGAGTTCCGCCAGACCTT | 20 | AsCpf1 RVR | 1142 |
| ADORA2A4481 | CCCTGGGGCTGGTGAGTGGA | 20 | AsCpf1RVR | 1143 |
| ADORA2A4482 | CCATCGGCCTGACTCCCATGC | 21 | Cas12a | 1174 |

It will be understood that the exemplary gRNAs disclosed herein are provided to illustrate non-limiting embodiments embraced by the present disclosure. Additional suitable gRNA sequences will be apparent to the skilled artisan based on the present disclosure, and the disclosure is not limited in this respect.

### Methods of Characterization

Methods of characterizing cells including characterizing cellular phenotype are known to those of skill in the art. In some embodiments, one or more such methods may include, but not be limited to, for example, morphological analyses and flow cytometry. Cellular lineage and identity markers are known to those of skill in the art. One or more such markers may be combined with one or more characterization methods to determine a composition of a cell population or phenotypic identity of one or more cells. For example, in some embodiments, cells of a particular population will be characterized using flow cytometry (for example, see Ye Li et al., Cell Stem Cell. 2018 Aug. 2; 23(2): 181-192.e5). In some such embodiments, a sample of a population of cells will be evaluated for presence and proportion of one or more cell surface markers and/or one or more intracellular markers. As will be understood by those of skill in the art, such cell surface markers may be representative of different lineages. For example, pluripotent cells may be identified by one or more of any number of markers known to be associated with such cells, such as, for example, CD34. Further, in some embodiments, cells may be identified by markers that indicate some degree of differentiation. Such markers will be known to one of skill in the art. For example, in some embodiments, markers of differentiated cells may include those associated with differentiated hematopoietic cells such as, e.g., CD43, CD45 (differentiated hematopoietic cells). In some embodiments, markers of differentiated cells may be associated with NK cell phenotypes such as, e.g., CD56, NK cell receptor immunoglobulin gamma Fc region receptor III (FcγRIII, cluster of differentiation 16 (CD16)), natural killer group-2 member D (NKG2D), CD69, a natural cytotoxicity receptor, etc. In some embodiments, markers may be T cell markers (e.g., CD3, CD4, CD8, etc.).

### Methods of Use

A variety of diseases, disorders and/or conditions may be treated through use of cells provided by the present disclosure. For example, in some embodiments, a disease, disorder and/or condition may be treated by introducing genetically modified or engineered cells as described herein (e.g., genetically modified NK or iNK cells) to a subject. Examples of diseases that may be treated include, but are not limited to, cancer, e.g., solid tumors, e.g., of the brain, prostate, breast, lung, colon, uterus, skin, liver, bone, pancreas, ovary, testes, bladder, kidney, head, neck, stomach, cervix, rectum, larynx, or esophagus; and hematological malignancies, e.g., acute and chronic leukemias, lymphomas, multiple myeloma and myelodysplastic syndromes.

In some embodiments, the present disclosure provides methods of treating a subject in need thereof by administering to the subject a composition comprising any of the cells described herein. In some embodiments, a therapeutic agent or composition may be administered before, during, or after the onset of a disease, disorder, or condition (including, e.g., an injury). In some embodiments, the present disclosure provides any of the cells described herein for use in the preparation of a medicament. In some embodiments, the present disclosure provides any of the cells described herein for use in the treatment of a disease, disorder, or condition, that can be treated by a cell therapy.

In particular embodiments, the subject has a disease, disorder, or condition, that can be treated by a cell therapy. In some embodiments, a subject in need of cell therapy is a subject with a disease, disorder and/or condition, whereby a cell therapy, e.g., a therapy in which a composition comprising a cell described herein, is administered to the subject, whereby the cell therapy treats at least one symptom associated with the disease, disorder, and/or condition. In some embodiments, a subject in need of cell therapy includes, but is not limited to, a candidate for bone marrow or stem cell transplant, a subject who has received chemotherapy or irradiation therapy, a subject who has or is at risk of having cancer, e.g., a cancer of hematopoietic system, a subject having or at risk of developing a tumor, e.g., a solid tumor, and/or a subject who has or is at risk of having a viral infection or a disease associated with a viral infection.

### Pharmaceutical Compositions

In some embodiments, the present disclosure provides pharmaceutical compositions comprising one or more genetically modified or engineered cells described herein, e.g., a genetically modified NK or iNK cell described herein. In some embodiments, a pharmaceutical composition further comprises a pharmaceutically acceptable excipient. In some embodiments, a pharmaceutical composition comprises isolated pluripotent stem cell-derived hematopoietic lineage cells comprising at least 50%, 60%, 70%, 80%, 90%, 95%, 98%, or 99% T cells, NK cells, NKT cells, CD34+ HE cells or HSCs, e.g., genetically modified (e.g., edited) T cells, NK cells, NKT cells, CD34+ HE cells or HSCs. In some embodiments, a pharmaceutical composition comprises isolated pluripotent stem cell-derived hematopoietic lineage cells comprising about 95% to about 100% T cells, NK cells, NKT cells, CD34+ HE cells or HSCs, e.g., genetically modified (e.g., edited) T cells, NK cells, NKT cells, CD34+ HE cells or HSCs.

In some embodiments, a pharmaceutical composition of the present disclosure comprises an isolated population of pluripotent stem cell-derived hematopoietic lineage cells, wherein the isolated population has less than about 0.1%, 0.5%, 1%, 2%, 5%, 10%, 15%, 20%, 25%, or 30% T cells, NK cells, NKT cells, CD34+ HE cells or HSCs, e.g., genetically modified (e.g., edited) T cells, NK cells, NKT cells, CD34+ HE cells or HSCs. In some embodiments, an isolated population of pluripotent stem cell-derived hematopoietic lineage cells has more than about 0.1%, 0.5%, 1%, 2%, 5%, 10%, 15%, 20%, 25%, or 30% T cells, NK cells, NKT cells, CD34+ HE cells or HSCs, e.g., genetically modified (e.g., edited) T cells, NK cells, NKT cells, CD34+ HE cells or HSCs. In some embodiments, an isolated population of pluripotent stem cell-derived hematopoietic lineage cells has about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% T cells, NK cells, NKT cells, CD34+ HE cells or HSCs, e.g., genetically modified (e.g., edited) T cells, NK cells, NKT cells, CD34+ HE cells or HSCs.

In some embodiments, an isolated population of pluripotent stem cell-derived hematopoietic lineage cells comprises about 0.1%, about 1%, about 3%, about 5%, about 10%, about 15%, about 20%, about 25%, about 30%, about 35%, about 40%, about 45%, about 50%, about 60%, about 70%, about 80%, about 90%, about 95%, about 98%, about 99%, or about 100% T cells, NK cells, NKT cells, CD34+ HE cells or HSCs, e.g., genetically modified (e.g., edited) T cells, NK cells, NKT cells, CD34+ HE cells or HSCs.

As one of ordinary skill in the art would understand, both autologous and allogeneic cells can be used in adoptive cell therapies. Autologous cell therapies generally have reduced infection, low probability for GVHD, and rapid immune reconstitution relative to other cell therapies. Allogeneic cell therapies generally have an immune mediated graft-versus-malignancy (GVM) effect, and low rate of relapse relative to other cell therapies. Based on the specific condition(s) of the subject in need of the cell therapy, one of ordinary skill in the art would be able to determine which specific type of therapy(ies) to administer.

In some embodiments, a pharmaceutical composition comprises pluripotent stem cell-derived hematopoietic lineage cells that are allogeneic to a subject. In some embodiments, a pharmaceutical composition comprises pluripotent stem cell-derived hematopoietic lineage cells that are autologous to a subject. For autologous transplantation, the isolated population of pluripotent stem cell-derived hematopoietic lineage cells can be either a complete or partial HLA-match with the subject being treated. In some embodiments, the pluripotent stem cell-derived hematopoietic lineage cells are not HLA-matched to a subject.

In some embodiments, pluripotent stem cell-derived hematopoietic lineage cells can be administered to a subject without being expanded ex vivo or in vitro prior to administration. In particular embodiments, an isolated population of derived hematopoietic lineage cells is modulated and treated ex vivo using one or more agents to obtain immune cells with improved therapeutic potential. In some embodiments, the modulated population of derived hematopoietic lineage cells can be washed to remove the treatment agent(s), and the improved population can be administered to a subject without further expansion of the population in vitro. In some embodiments, an isolated population of derived hematopoietic lineage cells is expanded prior to modulating the isolated population with one or more agents.

In some embodiments, an isolated population of derived hematopoietic lineage cells can be genetically modified according to the methods of the present disclosure to express a recombinant TCR, CAR or other gene product of interest. For genetically engineered derived hematopoietic lineage cells that express a recombinant TCR or CAR, whether prior to or after genetic modification of the cells, the cells can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

### Cancers

Any cancer can be treated using a cell or pharmaceutical composition described herein. Exemplary therapeutic targets of the present disclosure include cancer cells from the bladder, blood, bone, bone marrow, brain, breast, colon, esophagus, eye, gastrointestinal system, gum, head, kidney, liver, lung, nasopharynx, neck, ovary, prostate, skin, stomach, testis, tongue, or uterus. In addition, a cancer may specifically be of the following non-limiting histological type: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a breast cancer. In some embodiments, the cancer is colorectal cancer (e.g., colon cancer). In some embodiments, the cancer is gastric cancer. In some embodiments, the cancer is RCC. In some embodiments, the cancer is non-small cell lung cancer (NSCLC). In some embodiments, the cancer is head and neck cancer.

In some embodiments, solid cancer indications that can be treated with cells described herein (e.g., cells modified using methods of the disclosure, e.g., genetically modified iNK cells), either alone or in combination with one or more additional cancer treatment modality, include: bladder cancer, hepatocellular carcinoma, prostate cancer, ovarian/uterine cancer, pancreatic cancer, mesothelioma, melanoma, glioblastoma, HPV-associated and/or HPV-positive cancers such as cervical and HPV+ head and neck cancer, oral cavity cancer, cancer of the pharynx, thyroid cancer, gallbladder cancer, and soft tissue sarcomas. In some embodiments, hematological cancer indications that can be treated with cells described herein (e.g., cells modified using methods of the disclosure, e.g., genetically modified iNK cells), either alone or in combination with one or more additional cancer treatment modalities, include: ALL, CLL, NHL, DLBCL, AML, CML, and multiple myeloma (MM).

In some embodiments, examples of cellular proliferative and/or differentiative disorders of the lung that can be treated with cells described herein (e.g., cells modified using methods of the disclosure) include, but are not limited to, tumors such as bronchogenic carcinoma, including paraneoplastic syndromes, bronchioloalveolar carcinoma, neuroendocrine tumors, such as bronchial carcinoid, miscellaneous tumors, metastatic tumors, and pleural tumors, including solitary fibrous tumors (pleural fibroma) and malignant mesothelioma.

In some embodiments, examples of cellular proliferative and/or differentiative disorders of the breast that can be treated with cells described herein (e.g., cells modified using methods of the disclosure) include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

In some embodiments, examples of cellular proliferative and/or differentiative disorders involving the colon that can be treated with cells described herein (e.g., cells modified using methods of the disclosure) include, but are not limited to, tumors of the colon, such as non-neoplastic polyps, adenomas, familial syndromes, colorectal carcinogenesis, colorectal carcinoma, and carcinoid tumors.

In some embodiments, examples of cancers or neoplastic conditions, in addition to the ones described above that can be treated with cells described herein (e.g., cells modified using methods of the disclosure), include, but are not limited to, a fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, or Kaposi sarcoma.

In some embodiments, cells described herein (e.g., cells modified using methods of the disclosure) are used in combination with one or more cancer treatment modalities. In some embodiments, other cancer treatment modalities include, but are not limited to: chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1 (see, e.g., Agnew, Chem. Intl. Ed. Engl., 1994; 33:183-186); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylornithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

In some embodiments, cells described herein (e.g., cells modified using methods of the disclosure) are used in combination with one or more cancer treatment modalities that facilitate the induction of antibody dependent cellular cytotoxicity (ADCC) (see e.g., Janeway's Immunobiology by K. Murphy and C. weaver). In some embodiments, such a cancer treatment modality is an antibody. In some embodiments, such an antibody is Trastuzumab. In some embodiments, such an antibody is Rituximab. In some embodiments, such an antibody is Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Romosozumab, Risankizumab, Polatuzumab vedotin, Brolucizumab, or any combination thereof (see e.g., Lu et al., Development of therapeutic antibodies for the treatment of diseases. Journal of Biomedical Science, 2020). In some embodiments, cells described herein (e.g., cells modified using methods of the disclosure) are used in combination with one or more cancer treatment modalities that facilitate the induction of antibody dependent cellular cytotoxicity (ADCC), wherein the cancer treatment modality is an antibody or appropriate fragment thereof targeting CD20, TNFα, HER2, CD52, IgE, EGFR, VEGF-A, ITGA4, CTLA-4, CD30, VEGFR2, α4β7 integrin, CD19, CD3, PD-1, GD2, CD38, SLAMF7, PDGFRα, PD-L1, CD22, CD33, IFNγ, CD79β, or any combination thereof.

In some embodiments, cells described herein are utilized in combination with checkpoint inhibitors. Examples of suitable combination therapy checkpoint inhibitors include, but are not limited to, antagonists of PD-1 (Pdcdl, CD279), PDL-1 (CD274), TIM-3 (Havcr2), TIGIT (WUCAM and Vstm3), LAG-3 (Lag3, CD223), CTLA-4 (Ctla4, CD152), 2B4 (CD244), 4-1BB (CD137), 4-1BBL (CD137L), A2aR, BATE, BTLA, CD39 (Entpdl), CD47, CD73 (NT5E), CD94, CD96, CD160, CD200, CD200R, CD274, CEACAMI, CSF-1R, Foxpl, GARP, HVEM, IDO, EDO, TDO, LAIR-1, MICA/B, NR4A2, MAFB, OCT-2 (Pou2f2), retinoic acid receptor alpha (Rara), TLR3, VISTA, NKG2A/HLA-E, inhibitory KIR (for example, 2DL1, 2DL2, 2DL3, 3DL1, and3DL2), or any suitable combination thereof.

In some embodiments, the antagonist inhibiting any of the above checkpoint molecules is an antibody. In some embodiments, the checkpoint inhibitory antibodies may be murine antibodies, human antibodies, humanized antibodies, a camel Ig, a shark heavy chain-only antibody (VNAR), Ig NAR, chimeric antibodies, recombinant antibodies, or antibody fragments thereof. Non-limiting examples of antibody fragments include Fab, Fab′, F(ab)′2, F(ab)′3, Fv, single chain antigen binding fragments (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody, which may be more cost-effective to produce, more easily used, or more sensitive than the whole antibody. In some embodiments, the one, or two, or three, or more checkpoint inhibitors comprise at least one of atezolizumab (anti-PDL1 mAb), avelumab (anti-PDL1 mAb), durvalumab (anti-PDL1 mAb), tremelimumab (anti-CTLA4 mAb), ipilimumab (anti-CTLA4 mAb), IPH4102 (anti-KIR), IPH43 (anti-MICA), IPH33 (anti-TLR3), lirimumab (anti-KIR), monalizumab (anti-NKG2A), nivolumab (anti-PD1 mAb), pembrolizumab (anti-PD 1 mAb), and any derivatives, functional equivalents, or biosimilars thereof.

In some embodiments, the antagonist inhibiting any of the above checkpoint molecules is microRNA-based, as many miRNAs are found as regulators that control the expression of immune checkpoints (Dragomir et al., Cancer Biol Med. 2018, 15(2): 103-115). In some embodiments, the checkpoint antagonistic miRNAs include, but are not limited to, miR-28, miR-15/16, miR-138, miR-342, miR-20b, miR-21, miR-130b, miR-34a, miR-197, miR-200c, miR-200, miR-17-5p, miR-570, miR-424, miR-155, miR-574-3p, miR-513, miR-29c, and/or any suitable combination thereof.

In some embodiments, cells described herein (e.g., cells modified using methods of the disclosure) are used in combination with one or more cancer treatment modalities such as exogenous interleukin (IL) dosing. In some embodiments, an exogenous IL provided to a patient is IL-15. In some embodiments, systemic IL-15 dosing when used in combination with cells described herein is reduced when compared to standard dosing concentrations (see e.g., Waldmann et al., IL-15 in the Combination Immunotherapy of Cancer. Front. Immunology, 2020).

Other compounds that are effective in treating cancer are known in the art and described herein that are suitable for use with the compositions and methods of the present disclosure as additional cancer treatment modalities are described, for example, in the “Physicians' Desk Reference, 62nd edition. Oradell, N.J.: Medical Economics Co., 2008”, Goodman & Gilman's “The Pharmacological Basis of Therapeutics, Eleventh Edition. McGraw-Hill, 2005”, “Remington: The Science and Practice of Pharmacy, 20th Edition. Baltimore, Md.: Lippincott Williams & Wilkins, 2000,” and “The Merck Index, Fourteenth Edition. Whitehouse Station, N.J.: Merck Research Laboratories, 2006”, incorporated herein by reference in relevant parts.

All publications, patents and patent applications cited herein, whether supra or infra, are hereby incorporated by reference in their entirety.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially” of indicates that the listed elements are required or mandatory, but that no other elements are optional and may or may not be present depending upon whether or not they affect the activity or action of the listed elements.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. The contents of database entries, e.g., NCBI nucleotide or protein database entries provided herein, are incorporated herein in their entirety. Where database entries are subject to change over time, the contents as of the filing date of the present application are incorporated herein by reference. Aspects of the embodiments can be modified, if necessary to employ concepts of the various patents, applications and publications to provide yet further embodiments.

The disclosure is further illustrated by the following examples. The examples are provided for illustrative purposes only. They are not to be construed as limiting the scope or content of the disclosure in any way.

### EXAMPLES

### Example 1: Screening of Guide RNAs for GAPDH

This example describes the screening of AsCpf1 (AsCas12a) guide RNAs that target the housekeeping gene GAPDH. GAPDH encodes Glyceraldehyde-3-Phosphate Dehydrogenase, an essential protein that catalyzes oxidative phosphorylation of glyceraldehyde-3-phosphate in the presence of inorganic phosphate and nicotinamide adenine dinucleotide (NAD), an important energy-yielding step in carbohydrate metabolism. The guide RNAs used in this analysis were all 41-mer RNA molecules with the following design: 5′-UAAUUUCUACUCUUGUAGAU-[21-mer targeting domain sequence]-3′ (SEQ ID NO: 90). For example, the guide RNA denoted RSQ22337 had the following sequence:

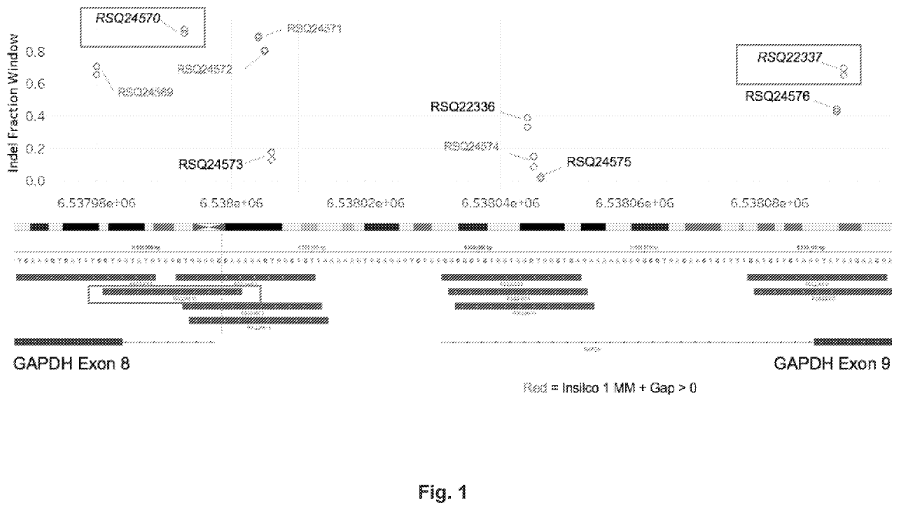

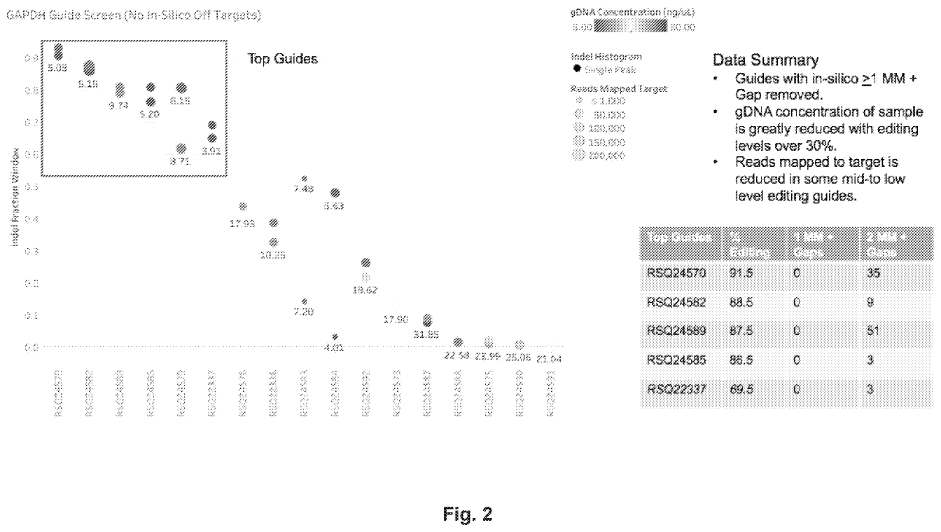

(SEQ ID NO: 93)
5′-UAAUUUCUACUCUUGUAGAUAUCUUCUAGGUAUGACAACGA-3′

where the 21-mer targeting domain sequence is underlined. The guide RNAs with the targeting domain sequences shown in Table 13 were tested to determine how effective they were at editing GAPDH. Cas12a RNPs (RNPs having an engineered Cas12a (SEQ ID NO: 62)), containing each of these guide RNAs were transfected into iPSCs, and then editing levels were assayed three days after transfection (see e.g., Wong, K. G. et al. CryoPause: A New Method to Immediately Initiate Experiments after Cryopreservation of Pluripotent Stem Cells. Stem Cell Reports 9, 355-365 (2017)). The results are shown in FIG. 1 and FIG. 2. RSQ24570, RSQ24582, RSQ24589, RSQ24585, and RSQ22337 exhibited the greatest levels of measurable editing out of the GAPDH guides tested, editing approximately 70% or more of cells (about 92%, 89%, 88%, 87%, and 70%, respectively). It was observed that cells transfected with gRNAs targeting certain exonic regions yielded much lower amounts of isolatable genomic DNA (gDNA) for analyzing editing efficiency (at day 3 after transfection) when compared to cells transfected with gRNAs targeting intronic regions, indicating that that RNPs with certain exon-targeting gRNAs were cytotoxic to the cells. This suggested that cells edited with gRNAs targeting exonic regions could result in significant cell death due to the introduction of indels within GAPDH leading to expression of a non-functional GAPDH protein or a protein with insufficient function. It was postulated that it might be possible to use a rescue plasmid to repair the gRNA-mediated cleavage site in GAPDH while also knocking in a gene cargo of interest in frame with the repaired GAPDH via HDR, thereby rescuing those cells in which GAPDH is repaired and the cargo of interest is successfully integrated (as shown in FIG. 1 and FIG. 2). Those transfected cells that are edited (the majority of transfected cells, if a highly effective RNA-guided nucleases is used) but do not undergo HDR repair of GAPDH and do not integrate the cargo of interest die over time because they do not have a functioning GAPDH gene. Those cells carrying the cargo of interest would have an advantage due to a fully functioning GAPDH gene as the cells grow and divide, and these cells would be selected for over time. The expected end result would be a population of cells with a very high rate of cargo knock-in within the GAPDH locus.

The data in FIG. 2 suggested that while Cas12a RNP comprising RSQ22337 resulted in an editing level of approximately 70% at 3 days post-transfection, it caused slightly higher levels of toxicity than other exonic guides (RSQ24570, RSQ24582, RSQ24589, and RSQ24585) (see FIG. 2, only about 3.9 ng/μL of gDNA was isolated from edited cells). Thus, the actual editing efficiency was very likely significantly higher than 70%, as many cells had already died by 3 days post-transfection due to the lack of available rescue constructs and NHEJ forming toxic indels. As a result, RSQ22337 was chosen for further testing.

TABLE 13
Guide RNA sequences

| SEQ ID | | gRNA targeting domain | |
|---|---|---|---|
| NO: | Name | sequence (RNA) | Location |
| 94 | RSQ22336 | UGAGCCAGCCACCAGAGGGCG | Intron 8 |
| 95 | RSQ22337 | AUCUUCUAGGUAUGACAACGA | Intron 8/Exon 9 (cut site |
| | | | in exon 9) |
| 96 | RSQ22338 | GCUACAGCAACAGGGUGGUGG | Exon 9 |
| 97 | RSQ24559 | CCAUAAUUUCCUUUCAAGGUG | Intron 7 |
| 98 | RSQ24560 | CUUUCAAGGUGGGGAGGGAGG | Intron 7 |
| 99 | RSQ24561 | AAGGUGGGGAGGGAGGUAGAG | Intron 7 |
| 100 | RSQ24562 | GCAGACCACAGUCCAUGCCAU | Exon 8 |
| 101 | RSQ24563 | CAGACCACAGUCCAUGCCAUC | Exon 8 |
| 102 | RSQ24564 | CCGGAGGGGCCAUCCACAGUC | Exon 8 |
| 103 | RSQ24565 | UAGACGGCAGGUCAGGUCCAC | Exon 8 |
| 104 | RSQ24566 | CUAGACGGCAGGUCAGGUCCA | Exon 8 |
| 105 | RSQ24567 | UCUAGACGGCAGGUCAGGUCC | Exon 8 |
| 106 | RSQ24568 | GCAGGUUUUUCUAGACGGCAG | Exon 8 |
| 107 | RSQ24569 | UCAAGCUCAUUUCCUGGUAUG | Exon 8 |
| 108 | RSQ24570 | CUGGUAUGUGGCUGGGGCCAG | Exon 8/Intron 8 (cut site |
| | | | in intron 8) |
| 109 | RSQ24571 | AGAGCCAGUCUCUGGCCCCAG | Intron 8 |
| 110 | RSQ24572 | AAGAGCCAGUCUCUGGCCCCA | Intron 8 |
| 111 | RSQ24573 | UAAGAGCCAGUCUCUGGCCCC | Intron 8 |
| 112 | RSQ24574 | CUGAGCCAGCCACCAGAGGGC | Intron 8 |
| 113 | RSQ24575 | UCUGAGCCAGCCACCAGAGGG | Intron 8 |
| 114 | RSQ24576 | CAUCUUCUAGGUAUGACAACG | Exon 9 |
| 115 | RSQ24578 | UUGAUGGUACAUGACAAGGUG | 1 kb_downstream |
| 116 | RSQ24579 | GAGGCCCUACCCUCAGUCUGA | 1 kb_downstream |
| 117 | RSQ24580 | CCUCUCCUCGCUCCAGUCCUA | 1 kb_downstream |
| 118 | RSQ24581 | CUCUCCUCGCUCCAGUCCUAG | 1 kb_downstream |
| 119 | RSQ24582 | GCCAACAGCAGAUAGCCUAGG | 1 kb_downstream |
| 120 | RSQ24583 | UGUGCCCUCGUGUCUUAUCUG | 1 kb_downstream |
| 121 | RSQ24584 | CCUAGAUGAAUCCUGCUUGAA | 1 kb_downstream |
| 122 | RSQ24585 | GGUACUUGGUUUACCUAGAUG | 1 kb_downstream |
| 123 | RSQ24586 | AGGUACUUGGUUUACCUAGAU | 1 kb_downstream |
| 124 | RSQ24587 | AAACAUUAUAUAGUCCUUACC | 1 kb_downstream |
| 125 | RSQ24588 | UAAACAUUAUAUAGUCCUUAC | 1 kb_downstream |
| 126 | RSQ24589 | CCGAUUUUUAAACAUUAUAUA | 1 kb_downstream |
| 127 | RSQ24590 | ACCGAUUUUUAAACAUUAUAU | 1 kb_downstream |
| 128 | RSQ24591 | UACCGAUUUUUAAACAUUAUA | 1 kb_downstream |
| 129 | RSQ24592 | AAAAUCGGUAAAAAUGCCCAC | 1 kb_downstream |
| 130 | RSQ24593 | GAGGAAGAUGAACUGAGAUGU | 1 kb_downstream |
| 131 | RSQ24594 | AGGAAGAUGAACUGAGAUGUG | 1 kb_downstream |

### Example 2: Rescue of GAPDH Knock-Out Through Targeted Integration

To test the feasibility of the exemplary selection system illustrated in FIGS. 3A, 3B, and 3C, the essential gene GAPDH was targeted in iPSCs using an RNP comprising AsCpf1 (SEQ ID NO: 62), and a guide RNA (RSQ22337 (SEQ ID NO: 95)), resulting in a double-strand break towards the 5′ end of the last exon of GAPDH (exon 9). While iPSCs were tested for the purposes of this experiment, the described methods could be applied to other cell types. RSQ22337 was determined to be highly specific to GAPDH and have minimal off-target sites in the genome (data not shown). GAPDH was thus considered a good exemplary candidate target gene for the cargo integration and selection methods described herein, at least in part because there was at least one highly specific gRNA targeting a terminal exon capable of mediating highly efficient RNA-guided cleavage.

The CRISPR/Cas nuclease and guide RNA were introduced into cells by nucleofection (electroporation) of a ribonucleoprotein (RNP) according to known methods. The cells were also contacted with a double stranded DNA donor template (e.g., a dsDNA plasmid) that included a knock-in cassette comprising in 5′-to-3′ order, a 5′ homology arm approximately 500 bp in length (comprising a portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for CD47 (“Cargo”), a stop codon and polyA signal sequence, and a 3′ homology arm approximately 500 bp in length (comprising a coding portion of exon 9 including a stop codon, the 3′ exonic region of exon 9, and a portion of the downstream intergenic sequence) (as shown in FIG. 3B). The 5′ and 3′ homology arms flanking the knock-in cassette were designed to correspond to sequences surrounding the RNP cleavage site.

As shown schematically in FIG. 3C, NHEJ-mediated creation of indels in cells that are edited by the DNA nuclease but not successfully targeted by the DNA donor template, produce a non-functional version of GAPDH which is lethal to the cells. This knock-out is “rescued” in cells that are successfully targeted by the DNA donor template by correct integration of the knock-in cassette, which restores the GAPDH coding region so that a functioning gene product is produced, and positions the P2A-Cargo sequence in frame with and downstream (3′) of the GAPDH coding sequence. These cells survive and continue to proliferate. Cells that are not edited by the DNA nuclease also continue to proliferate but are expected to represent a very small percentage of the overall cell population, if, as in this case, the editing efficiency of the nuclease in combination with the gRNA is high (see Example 1) and results in creation of a non-functional protein. The editing results for RSQ22337 likely underestimate the actual editing efficiency of the guide due to cell death within the population of edited cells.

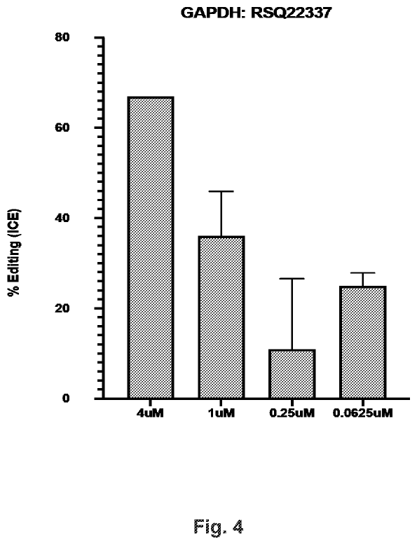

The editing efficiency of RNPs containing RSQ22337 were tested at different concentrations (4 μM, 1 μM, 0.25 μM, or 0.0625 μM of RNP) in the absence of double stranded DNA donor template) was first measured at 48 after nucleofection of iPSCs (a time point prior to cell death due to loss of GAPDH gene function). The results show that a concentration of 4 μM resulted in the highest editing levels (see FIG. 4).

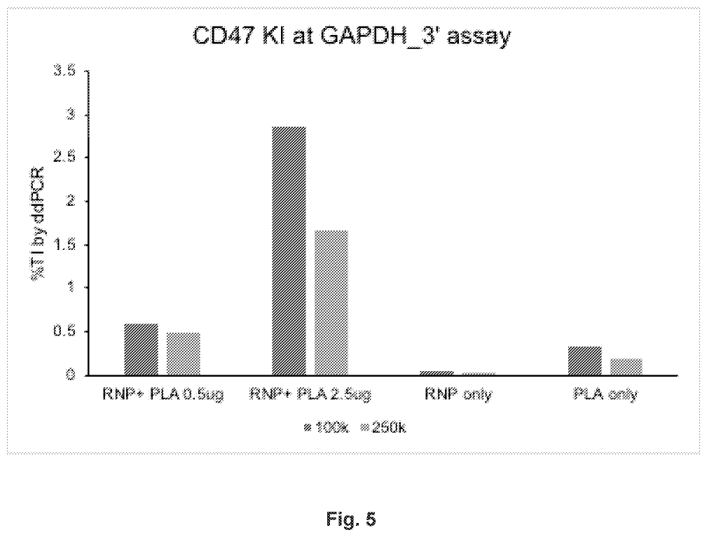

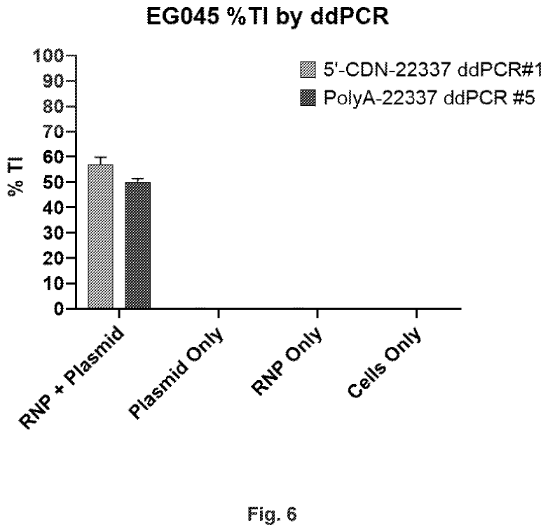

FIGS. 5 and 6 show that a protein-encoding cargo gene can be knocked into a housekeeping gene, such as GAPDH, at high efficiency using the selection systems described herein. FIG. 5 shows the knock-in (KI) efficiency of the CD47-encoding “cargo” in GAPDH at 4 days post-electroporation when RNP was present at a concentration of 4 μM and the dsDNA plasmid (“PLA”) encoding CD47 was also present. Knock-in efficiency was measured with two different concentrations of the plasmid (0.5 μg and 2.5 μg of plasmid) and found to be dose responsive. Knock-in was measured using ddPCR targeting the 3′ position of the knock-in “cargo”. Control cells electroporated with RNP alone or PLA alone exhibited much lower knock-in rates than electroporation of RNP and PLA (at a concentration of 2.5 μg).

FIG. 6 shows the knock-in efficiency of the CD47-encoding “cargo” in GAPDH at 9 days post-electroporation of the cells with the RNP and dsDNA plasmid encoding CD47. The percentage knock-in was similar when either the 5′ end or the 3′ end of the cargo was assayed by ddPCR, using a primer specific for the 5′ of the gRNA target site or 3′ of the site in the poly A region, increasing the reliability of the result. The knock-in efficiency of the cargo was significantly higher at 9 days compared to at 4 days post-transfection (compare FIGS. 5 and 6), consistent with the expectation that there would be substantial cell death in RNP-induced GAPDH knock-out cells that lacked a functional GAPDH gene as a result of unsuccessful cargo knock-in and rescue at GAPDH.

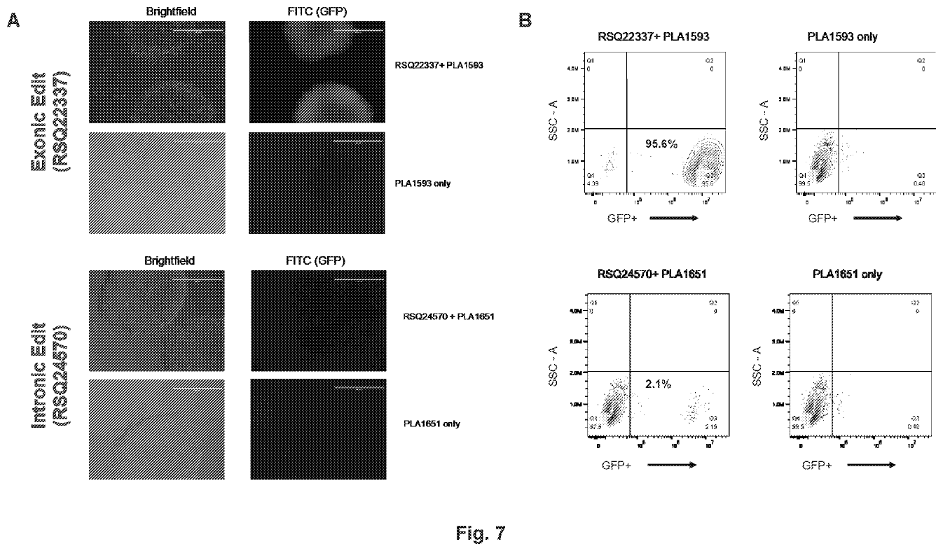

An experiment was then conducted to test the mechanism of the selection system described above by confirming that edited cells containing a successfully knocked-in cargo gene would be more efficiently selected for using a gRNA targeting a protein-coding exonic portion of GAPDH rather than a gRNA targeting an intron. FIG. 7 compares the knock-in efficiency of a GFP-encoding “cargo” knock-in cassette at the GAPDH locus when using a gRNA that mediates cleavage within an intron (RSQ24570 (SEQ ID NO: 108) binds to the exon 8-intron 9 junction, leading to Cas12a-mediated cleavage within intron 8) relative to a gRNA specific for an exon (RSQ22337 (SEQ ID NO: 95), targeting the intron 8-exon 9 junction, leading to Cas12a-mediated cleavage within exon 9). Rescue dsDNA plasmid PLA1593 comprising the reporter “cargo” GFP was nucleofected into iPSCs with an RNP (Cas12a and RSQ22337) targeting GAPDH as described above, while dsDNA plasmid PLA1651 comprising a donor template sequence as depicted in SEQ ID NO: 46 was nucleofected with an RNP comprising Cas12a and RSQ24570. The homology arms of each plasmid were designed to mediate HDR based on the target site of each gRNA. Knock-in was visualized using microscopy (FIG. 7A) and was measured using flow cytometry (FIG. 7B). Knock-in efficiency was significantly higher when using a gRNA and associated knock-in cassette that cleaves at an exonic coding region (exon 9) when compared to an intronic region (intron 8). FIG. 7B shows that 95.6% of cells electroporated with RSQ22337 and the GFP-encoding “cargo” knock-in cassette (e.g., PLA1593; comprising donor template SEQ ID NO: 44) expressed GFP compared to only 2.1% of cells electroporated with RSQ24570 and a GFP-encoding “cargo” knock-in cassette (PLA1651; comprising donor template SEQ ID NO: 46). The results depicted in FIG. 7 are striking, as while the measured editing efficiency (as determined by indel generation frequency 72 hours post-transfection as discussed above in Example 1, see FIG. 2) of RSQ24570 is higher than that of RSQ22337, the proportion of cells rescued by the knock-in construct targeting the coding exonic region are significantly higher.

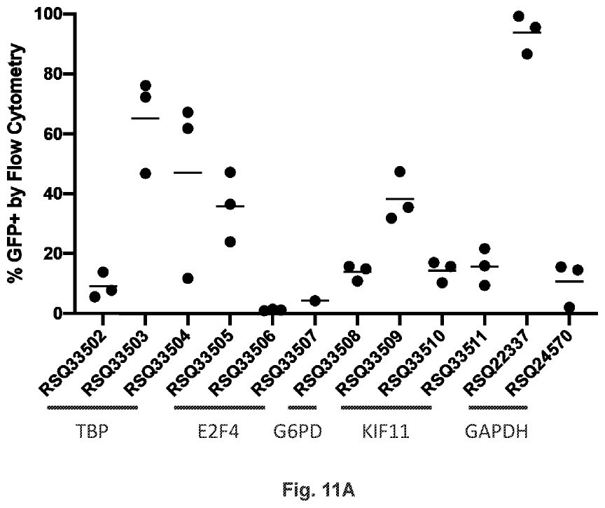

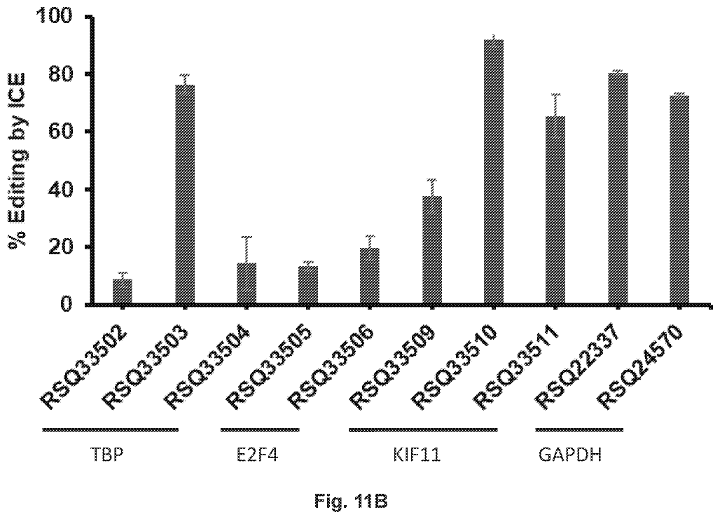

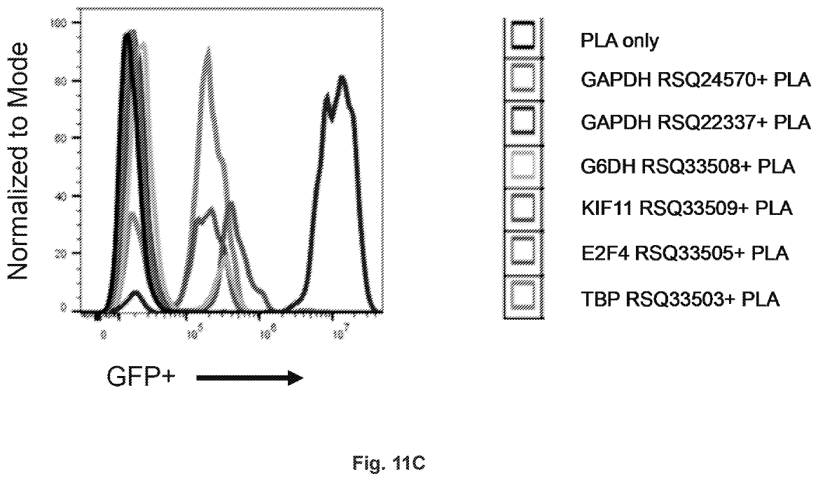

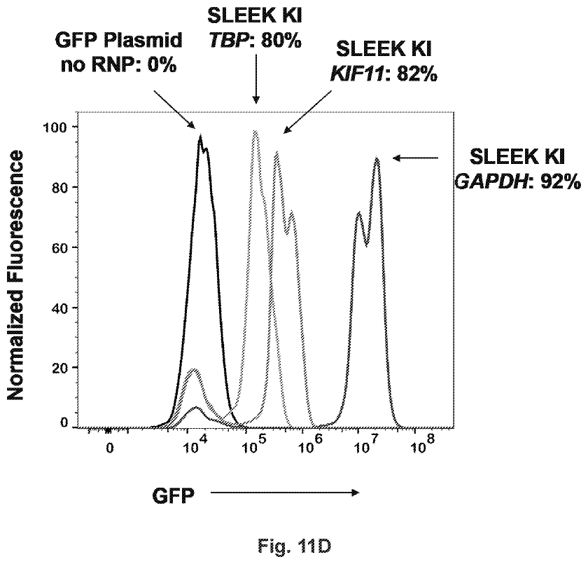

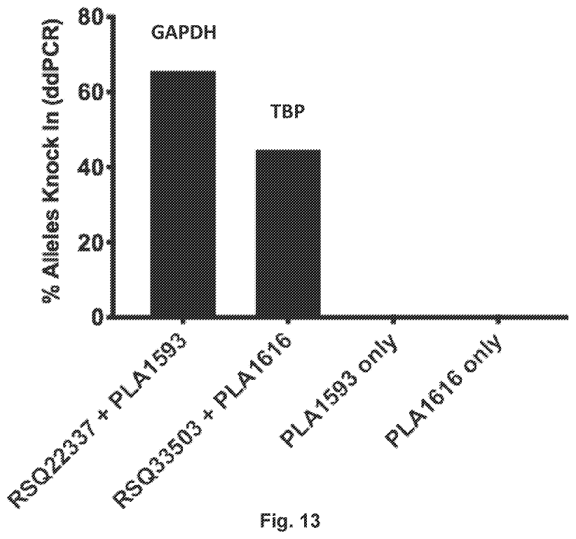

In an additional set of experiments, iPS cells were contacted with an RNP containing AsCas12a (SEQ ID NO: 62), and RSQ22337 (SEQ ID NO: 95) or RSQ24570 (SEQ ID NO: 108), along with either the PLA1593 (comprising donor template SEQ ID NO: 44) or the PLA1651 (comprising donor template SEQ ID NO: 46) double stranded DNA donor template plasmid, respectively, as described above. Flow cytometry was performed 7 days following nucleofection to detect GFP expression and help determine to what extent each plasmid mediated donor template and knock-in cassette was integrated successfully at its respective GAPDH target site. The GAPDH results in FIG. 11A show that cells nucleofected with the RNP containing RSQ22337 exhibited a much higher amount of GFP expression relative to cells nucleofected with RSQ24750, showing that most cells express GFP at day 7 following electroporation. This suggests that the GFP-encoding knock-in cassette integrated successfully at high levels within the RSQ22337-transfected cells. Cells nucleofected with RNPs containing RSQ24750 displayed much lower GFP expression, indicating that the knock-in cassette did not integrate successfully in most of these cells (FIG. 11A). The GAPDH results of FIG. 11B show that use of RSQ22337 resulted in about 80% editing as measured using genomic DNA 48 hours following RNP transfection, while RSQ24570 resulted in about 75% editing as measured using genomic DNA 48 hours following RNP transfection. The high editing of RSQ22337 correlated well with the high GFP expression level depicted in FIG. 11A; however, the high editing of RSQ24750 correlated poorly with the low GFP expression level depicted in FIG. 11A. FIG. 11C and FIG. 11D (representing an additional experiment where RSQ22337 was again used for editing at the GAPDH locus) show the relative integrated “cargo” (GFP) expression intensity of the edited cells. Finally, a ddPCR assay was conducted to determine the percentage of knock-in integration events in GAPDH alleles in the cells nucleofected with RNPs containing RSQ22337 and the PLA1593 donor plasmid. FIG. 13 shows by ddPCR that over 60% of alleles had a GFP-encoding cassette knocked-in successfully.

### Example 3: Rescue of GAPDH Knock-Out Through Targeted Integration of Multiple Cargos

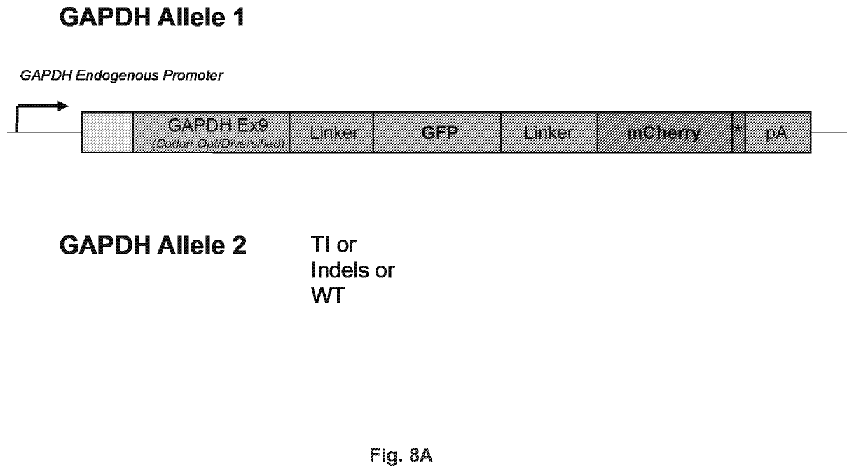

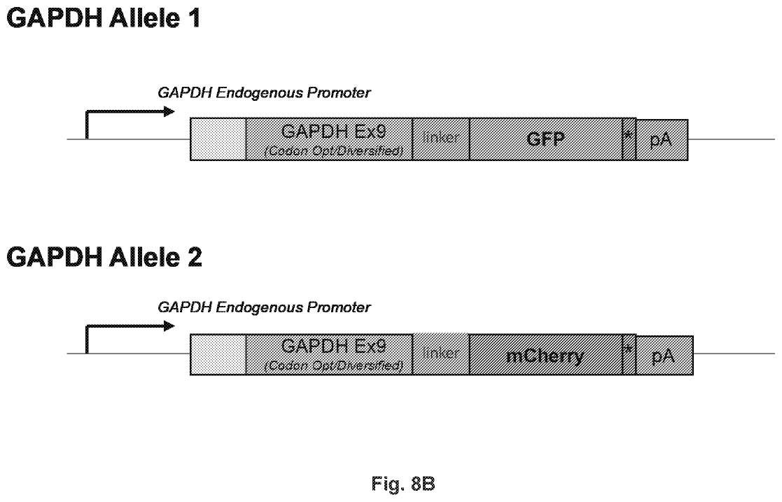

In some cases, it is desirable to use the selection and cargo knock in strategies disclosed herein to efficiently produce and isolate an edited cell containing two or more different exogenous coding sequences, e.g., two or more different exogenous genes, integrated into a single essential gene locus, such as, e.g., the GAPDH locus. FIG. 8 shows two strategies for introducing two or more different exogenous coding regions into an essential gene locus. FIG. 8A shows a first exemplary strategy wherein a multi-cistronic knock-in cassette, e.g., a bi-cistronic knock-in cassette containing two or more coding regions (GFP and mCherry in FIG. 8A), separated by linkers (e.g., T2A, P2A, and/or IRES; see SEQ ID NO: 29-32 and 33-37), is inserted into one or both of the alleles of the essential gene, e.g., GAPDH. FIG. 8B shows a second exemplary strategy (a bi-allelic insertion strategy) wherein two knock-in cassettes comprising different cargo sequences (e.g., different exogenous genes, such as GFP and mCherry in FIG. 8B) are inserted into separate alleles of the essential gene locus, e.g., GAPDH.

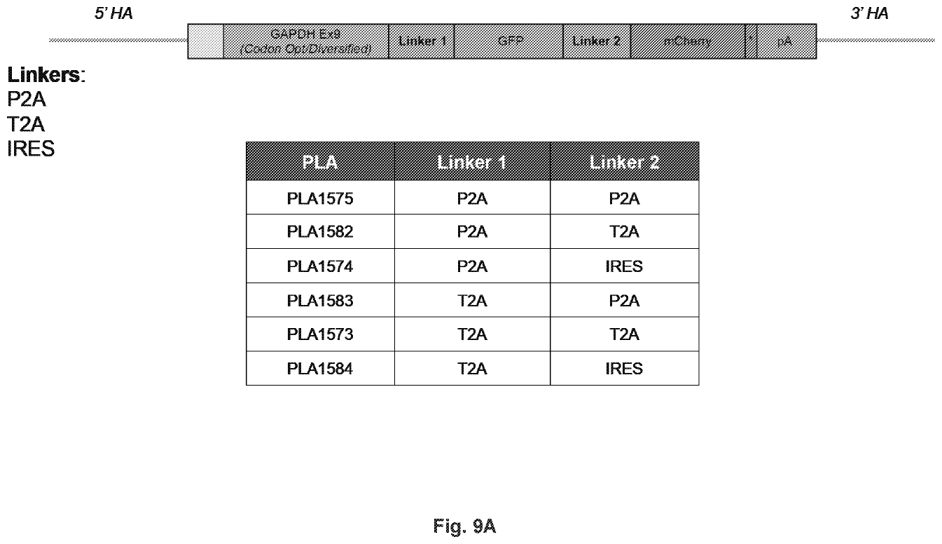

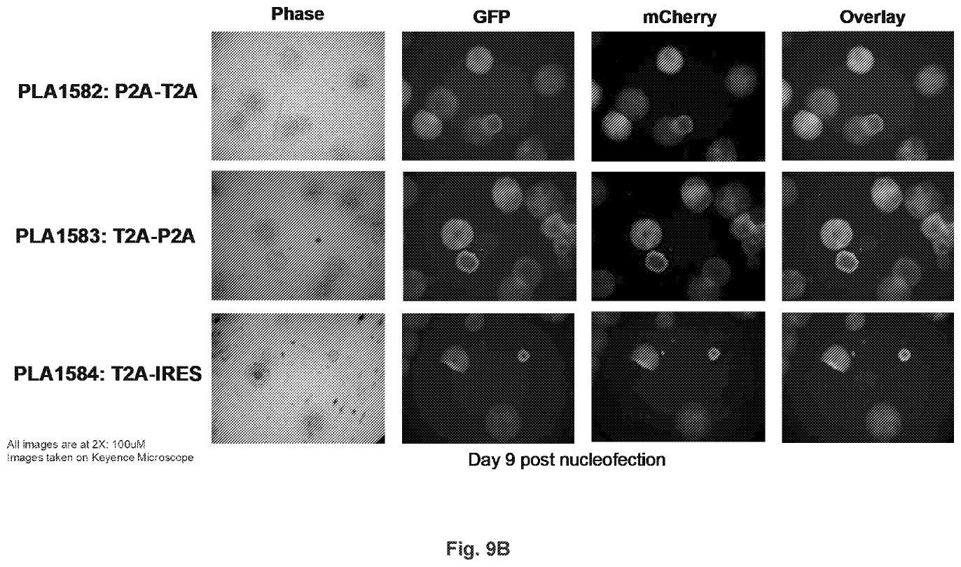

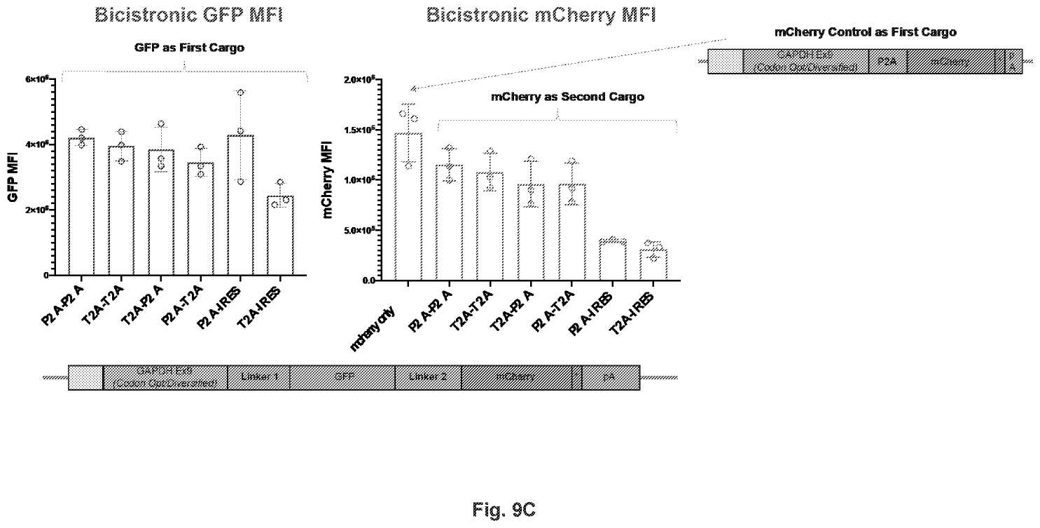

Experiments were conducted to test the integration strategy depicted in FIG. 8A, and to determine whether the use of different combinations of linkers in the knock-in cassette could affect the expression of the cargo sequences. An RNP containing Cas12a and RSQ22337 (targeting the GAPDH locus, as described in Examples 1 and 2) was nucleofected into iPSCs with one of six different plasmids (PLA) containing a bi-cistronic knock-in cassette comprising “cargo” sequences encoding GFP and mCherry (PLA1573, PLA1574, PLA1575, PLA1582, PLA1583, and PLA1584, as depicted in FIG. 9A; comprising donor templates SEQ ID NOs: 38-43). GFP was the first cargo and mCherry was the second cargo in each of these constructs. Each of the tested plasmids contained a different combination of linkers between the coding sequences (Linkers 1 and 2, as depicted in FIG. 9A). PLA1573 (comprising donor template SEQ ID NO: 38) contained T2A and T2A as linkers 1 and 2, respectively; PLA1574 (comprising donor template SEQ ID NO: 39) contained P2A and IRES as linkers 1 and 2, respectively; PLA1575 (comprising donor template SEQ ID NO: 40) contained P2A and P2A as linkers 1 and 2, respectively; PLA1582 (comprising donor template SEQ ID NO: 41) contained P2A and T2A as linkers 1 and 2, respectively; PLA1583 (comprising donor template SEQ ID NO: 42) contained T2A and P2A as linkers 1 and 2, respectively; and PLA1584 (comprising donor template SEQ ID NO: 43) contained T2A and IRES as linkers 1 and 2, respectively. FIG. 9B and FIG. 9C shows the results of various knock-in cassette integration events at the GAPDH locus. FIG. 9B depicts exemplary microscopy images (brightfield and fluorescent microscopy at 2× on a Keyence microscope) of edited iPSCs nine days following nucleofection with exemplary plasmids PLA1582, PLA1583, and PLA1584, each of which exhibited detectable GFP and mCherry expression.

FIG. 9C quantifies the fluorescence levels of GFP and mCherry in the iPSCs nucleofected with the various plasmids described in FIG. 9A containing the bi-cistronic knock-in cassettes with the different described linker pairs (PLA1575, PLA1582, PLA1574, PLA1583, PLA1573, and PLA1584). In each of these bi-cistronic constructs, GFP was always the first cargo and mCherry was always the second cargo. A plasmid containing a knock-in cassette with mCherry as a sole “cargo” (as depicted in FIG. 9C) was also tested as a control. The data show that the expression levels of GFP, as the first cargo, were similar between bicistronic constructs and consistently higher than the expression levels of mCherry, the second cargo. Cells containing the control knock-in cassette containing mCherry as the sole cargo exhibited the highest mCherry expression, suggesting that it is possible to vary (e.g., reduce) expression of a cargo by placing it as the second cargo in a bicistronic cassette. In addition, FIG. 9C shows that placement of an IRES linker immediately prior to the second cargo coding sequence resulted in lower expression of the second cargo when compared to the placement of a P2A or T2A linker prior to the second cargo coding sequence. Thus, the results show that it is possible to differentially modulate (i.e., increase or decrease) the expression of two cargo coding sequences from a multicistronic knock-in cassette by varying the order of the cargos in the cassette (placing a cargo as the first cargo for higher expression, or as the second cargo for lower expression) and by placing particular linkers (P2A or T2A for higher expression; IRES for lower expression) upstream of each of the cargos.

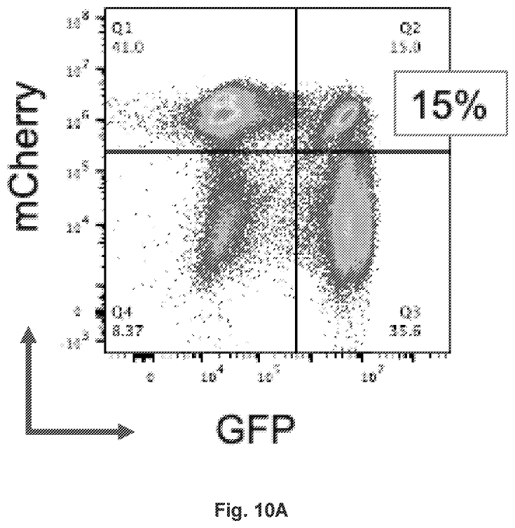

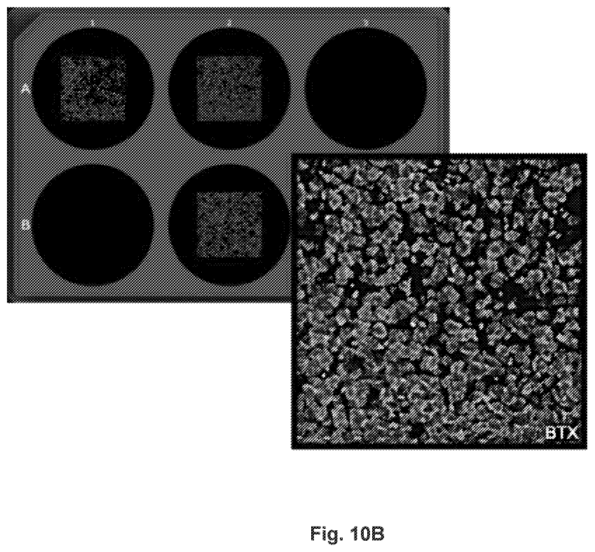

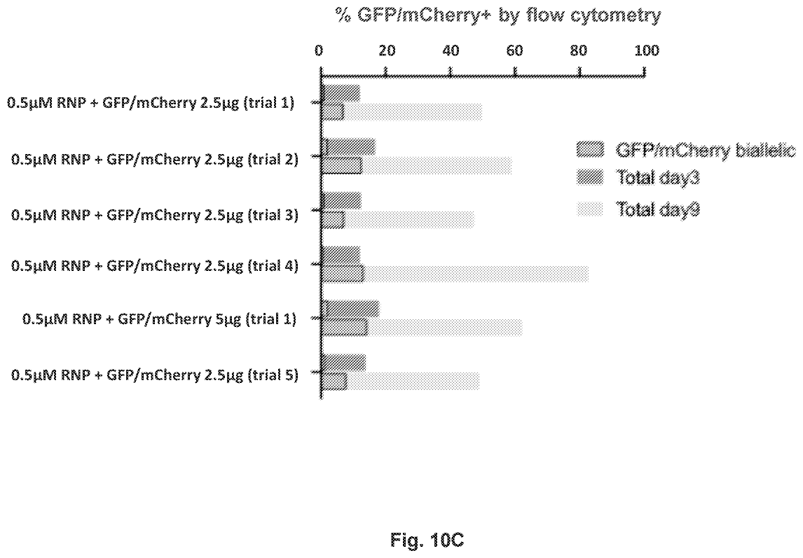

An experiment was conducted to test the bi-allelic integration strategy depicted in FIG. 8B. An RNP containing Cas12a and RSQ22337 (targeting the GAPDH locus, as described in Examples 1 and 2) was nucleofected into iPSCs with two different plasmids. One plasmid contained a knock-in cassette containing a GFP coding sequence as the cargo, and the second plasmid contained a knock-in cassette containing an mCherry coding sequence as the cargo (as depicted in FIG. 8B). FIG. 10A shows exemplary flow cytometry data for the nucleofected iPSCs. Gating showed that a high percentage, approximately 15%, of the nucleofected cells expressed GFP and mCherry, suggesting that the GFP knock-in cassette and the mCherry knock-in cassette were each integrated into an allele of GAPDH. Approximately 41% of the nucleofected cells expressed mCherry and approximately 36% of the nucleofected cells expressed GFP.

An additional experiment was conducted to test biallelic insertion of GFP and mCherry in populations of iPSCs. The iPSC populations were transformed as described. The cells were nucleofected with 0.5 μM RNPs comprising Cas12a and RSQ22337 (targeting the GAPDH locus, as described in Examples 1 and 2), and 2.5 μg of donor template (5 trials) or 5 μg of donor template (1 trial), and then sorted 3 or 9 days following nucleofection. An exemplary image of the edited cell populations that were analyzed by flow cytometry analysis is depicted in FIG. 10B. FIG. 10C provides the flow cytometry analysis results from these trials. The larger bar at each time point (day 3 or day 9) in FIG. 10C represents the total percentage of the cells in each population that positively express at least one cargo, e.g., at least one allele of GFP and/or at least one allele of mCherry cargo. The smaller bar at each time point shows the percentage of cells in each population that express both GFP and mCherry and therefore represents cells with GFP/mCherry biallelic integration. These results showed that approximately 8-15% percent of the transformed cells in each population displayed a biallelic GFP/mCherry insertion phenotype at nine days following transformation.

### Example 4: Rescue of B2M Knock-Out Through Targeted Integration

The approach described in Example 2 is used to target the B2M gene in NK cells (e.g., by targeting NK cells such as iPS-derived NK cells directly or iPS cells that are then differentiated into NK cells). NK cells that lack a functional B2M gene will not be able to recognize MHC Class I on the surface of one another and will attack each other, depleting the population in a phenomenon known as fratricide. By knocking-out the B2M gene and knocking-in a “cargo” sequence that also restores a functional B2M gene one automatically enriches for the knock-in cell type.

### Example 5: Rescue of TBP Knock-Out Through Targeted Integration

The knock-in integration and selection approach described in Example 2 was used to target the TBP gene in iPSCs. While iPSCs were tested for the purposes of this experiment, the described methods could be applied to other cell types. The TBP gene encodes TATA-box binding protein, a transcriptional regulator that plays a key role in the transcription initiation apparatus. AsCpf1 (AsCas12a) guide RNAs that target terminal exons of the TBP gene are shown in Table 14 below. The guide RNAs are all 41-mer RNA molecules with the following design: 5′-UAAUUUCUACUCUUGUAGAU-[21-mer targeting domain sequence]-3′ (SEQ ID NO: 90).

TABLE 14
Guide RNA sequences

| | | gRNA targeting domain | | |
|---|---|---|---|---|
| Name | Target Site | sequence (RNA) | Location | Plasmid |
| TBP-1 | RSQ33502 | AAAUGCUUCAUAAAUUUCUGC | Isoform 1 exon 8; | PLA1615 |
| | (SEQ ID | | isoform 2 exon 7 | |
| | NO: 148 | | | |
| TBP-2 | RSQ33503 | UGCUCUGACUUUAGCACCUAA | Isoform 1 exon 8; | PLA1616 |
| | (SEQ ID | | isoform 2 exon 7 | |
| | NO: 149) | | | |
| TBP-3 | RSQ33504 | AAAACAUCUACCCUAUUCUAA | Isoform 1 exon 8; | PLA1617 |
| | (SEQ ID | | isoform 2 exon 7 | |
| | NO: 150) | | | |

RSQ33502, RSQ33503, and RSQ33504 (SEQ ID NO: 148-150) described in Table 14 were each determined to be highly specific to TBP and have minimal off-target sites in the genome (data not shown). The TBP gene was thus considered a good candidate gene target for the cargo integration and selection methods described herein at least in part because there are gRNAs available capable of very specifically targeting a terminal exon (mRNA isoform 1 exon 8, or mRNA isoform 2 exon 7 respectively). However, for any of these gRNAs to be highly suitable for the methods described herein, they need to be highly effective at introducing indels at a location in the TBP locus that would knock out and/or severely reduce gene function.

Each of these gRNAs was then tested to determine whether it could be used to knock-in a cassette comprising a portion of TBP and an in-frame cargo sequence encoding GFP into a terminal exon of the TBP gene of cells, in the process rescuing the lethal phenotype that would otherwise result by introducing RNP-induced indels into the coding region of this essential gene. If the tested gRNA was effective at introducing indels at a location of TBP important for function at a high frequency, then transfected cells that do not undergo HDR to incorporate the knock-in cassette would be expected to die, resulting in a large population of the cells expressing GFP from the TBP locus. Specifically, iPSC cells were contacted with an RNP containing AsCas12a (SEQ ID NO: 62), and RSQ33502, RSQ33503 or RSQ33504 (SEQ ID NOs: 148-150), along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of a portion of the final TBP exon coding sequence (mRNA isoform 1 exon 8, or mRNA isoform 2 exon 7 respectively) and a sequence encoding the P2A self-cleaving peptide (“P2A”), similar to the dsDNA plasmid described in Example 2 for GAPDH. The TBP sequence in the double stranded DNA donor templates (PLA1615, PLA1616, or PLA1617; comprising donor template SEQ ID NOs: 47, 49, or 50) was codon optimized to prevent further binding by the accompanying guide RNA molecule (RSQ33502, RSQ33503 or RSQ33504). The knock-in cassette also included 3′ UTR and polyA signal sequences downstream of the Cargo sequence. An RNP containing RSQ33502 was administered with PLA1615 (comprising donor template SEQ ID NO: 47); RSQ33503 was administered with PLA1616 (comprising donor template SEQ ID NO: 49); and RSQ33504 was administered with PLA1617 (comprising donor template SEQ ID NO: 50). Each particular dsDNA plasmid (PLA) contained a donor template with homology arms and a knock-in cassette designed to specifically encompass and render ineffective the particular gRNA target site following knock-in cassette integration.

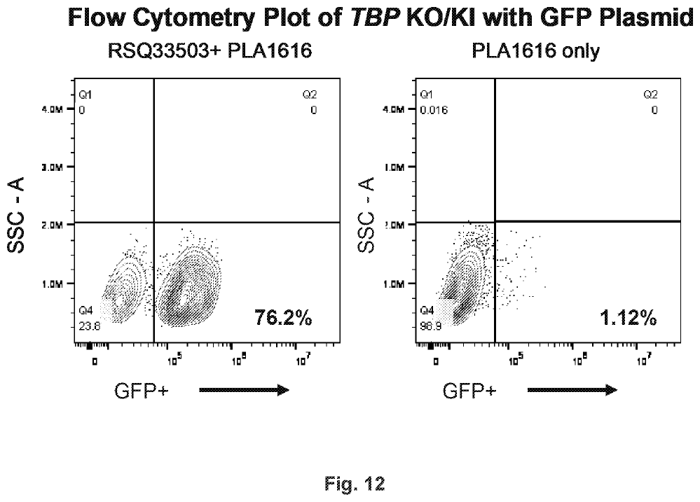

Flow cytometry was performed 7 days following nucleofection and was used to help determine to what extent each plasmid based knock-in cassette was integrated successfully at its respective TBP target site. FIG. 11A shows that cells nucleofected with RNPs containing RSQ33503 exhibited the greatest amounts of GFP expression relative to cells nucleofected with the other RNPs, suggesting that the GFP-encoding knock-in cassette integrated successfully at high levels within these cells. FIG. 12 shows that approximately 76% of the cells nucleofected with RNPs containing RSQ33503 (SEQ ID NO: 149) and the PLA1616 (comprising donor template SEQ ID NO: 49) plasmid expressed GFP compared to only about 1% of cells nucleofected with the PLA1616 plasmid alone (no RNP control). Cells nucleofected with RNPs containing RSQ33504 (SEQ ID NO: 150) also exhibited high levels of GFP expression, also suggesting higher knock-in cassette integration levels (FIG. 11A). Cells nucleofected with RNPs containing RSQ33502 (SEQ ID NO: 148) displayed much lower GFP expression, indicating that the knock-in cassette did not integrate successfully in most of these cells (FIG. 11A). FIG. 11B shows that use of the RNP containing RSQ33503 (SEQ ID NO: 149) resulted in about 80% editing, which correlated with the higher GFP expression level depicted in FIG. 11A. The percentage editing was measured two days following transfection and was determined by ICE assays (as described in Hsiau et al., Inference of CRISPR Edits from Sanger Trace Data. BioRxiv, 251082, August 2019). Use of the RNP containing RSQ33502 (SEQ ID NO: 148) resulted in a relatively low editing percentage, which correlated with the low GFP expression in FIG. 11A. FIG. 11C and FIG. 11D (representing an additional experiment where RSQ33503 was again used for editing at the TBP locus) show the relative integrated “cargo” (GFP) expression intensity of the edited cells. Finally, a ddPCR assay was conducted to determine the percent knock-in of the GFP cargo into the TBP alleles of the cells nucleofected with RNPs containing RSQ33503 (SEQ ID NO: 149) and the PLA1616 donor plasmid (comprising donor template SEQ ID NO: 49). FIG. 13 shows by ddPCR that over 40% of the TBP alleles had the GFP-encoding cassette successfully knocked-in.

### Example 6: Rescue of E2F4 Knock-Out Through Targeted Integration

The knock-in integration and selection approach described in Example 2 was used to target the E2F4 gene in iPSCs. While iPSCs were tested for the purposes of this experiment, the described methods could be applied to other cell types. The E2F4 gene encodes E2F Transcription Factor 4. This transcriptional regulator plays a key role in cell cycle regulation. AsCpf1 (AsCas12a) guide RNAs that target terminal exons of the E2F4 gene are shown in Table 15 below. The guide RNAs are all 41-mer RNA molecules with the following design: 5′-UAAUUUCUACUCUUGUAGAU-[21-mer targeting domain sequence]-3′ (SEQ ID NO: 90).

TABLE 15
Guide RNA sequences

| | | gRNA targeting domain | | |
|---|---|---|---|---|
| Name | Target Site | sequence (RNA) | Location | Plasmid |
| E2F4-1 | RSQ33505 | CCCCUCUGCUUCGUCUUUCUC | Exon 10 | PLA1626 |
| | (SEQ ID NO: 151) | | | |
| E2F4-2 | RSQ33506 | UCCACCCCCGGGAGACCACGA | Exon 10 | PLA1627 |
| | (SEQ ID NO: 152) | | | |
| E2F4-3 | RSQ33507 | AUGUGCCUGUUCUCAACCUCU | Exon 10 | PLA1628 |
| | (SEQ ID NO: 153) | | | |

RSQ33505, RSQ33506, and RSQ33507 (SEQ ID NOs: 151-153) were each determined to be highly specific to E2F4 and have minimal off-target sites in the genome (data not shown). The E2F4 gene was thus considered a good candidate gene target for the cargo integration and selection methods described herein at least in part because there are gRNAs available that are capable of very specifically targeting a terminal exon (exon 10). However, for any of these gRNAs to be highly suitable for the methods described herein, they need to be highly effective at introducing indels at a location in the E2F4 locus that would knock out or severely reduce gene function.

The gRNAs RSQ33505, RSQ33506, and RSQ33507 (SEQ ID NOs: 151-153) were then tested to determine whether they could be used to knock-in a cassette comprising a portion of E2F4 and a cargo sequence encoding GFP into a terminal exon of the E2F4 locus of cells, in the process rescuing the lethal phenotype that would otherwise result by introducing RNP-induced indels into the coding region of this essential gene at a high frequency. Specifically, iPSCs were contacted with an RNP containing AsCas12a (SEQ ID NO: 62), and RSQ33505, RSQ33506, or RSQ33507 (SEQ ID NOs: 151-153) along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of the final F2F4 exon coding sequence (exon 10) and a sequence encoding the P2A self-cleaving peptide (“P2A”), similar to the dsDNA plasmid described in Example 2 for GAPDH. The E2F4 sequence in the double stranded DNA donor templates (PLA1626, PLA1627, or PLA1628; comprising donor template SEQ ID NOs: 52-54) was codon optimized to prevent further binding by the accompanying guide RNA molecule (RSQ33505, RSQ33506 or RSQ33507; SEQ ID NOs: 151-153). The knock-in cassette also included 3′ UTR and polyA signal sequences downstream of the Cargo sequence. An RNP containing RSQ33505 (SEQ ID NO: 151) was administered with PLA1626 (comprising donor template SEQ ID NO: 52); RSQ33506 (SEQ ID NO: 152) was administered with PLA 1627 (comprising donor template SEQ ID NO: 53); and RSQ33507 (SEQ ID NO: 153) was administered with PLA1628 (comprising donor template SEQ ID NO: 54). Each particular dsDNA plasmid (PLA) contained a donor template with homology arms and a knock-in cassette designed to specifically encompass and render ineffective the particular gRNA target site following integration.

Flow cytometry was performed 7 days following nucleofection and was used to help determine to what extent each plasmid based knock-in cassette was integrated successfully at its respective E2F4 target site. FIG. 11A shows that cells nucleofected with RNPs containing RSQ33505 (SEQ ID NO: 151) exhibited the greatest amount of GFP expression relative to cells nucleofected with the other RNPs targeting E2F4, suggesting that the GFP-encoding knock-in cassette integrated successfully in many of these cells. Cells nucleofected with RNPs containing RSQ33506 or RSQ33507 (SEQ ID NOs: 152 and 153) displayed much lower GFP expression, indicating that the knock-in cassette did not integrate successfully in most of these cells (FIG. 11A). FIG. 11B shows that use of RNP containing RSQ33505 (SEQ ID NO: 151) or RSQ33506 (SEQ ID NO: 152) resulted in approximately 15% and approximately 20% editing rates respectively, when measured 48 hours after RNP transfection. The relatively lower observed editing rate for RSQ33505 (SEQ ID NO: 151) may be considered to unexpectedly correlate with a relatively high level of GFP integration in E2F4 (as observed in FIG. 11A), and could partially be the result of significant death within the population of edited cells at 48 hours. The percentage editing was measured two days following transfection and was determined by ICE assays (as described in Hsiau et al., August 2019). FIG. 11C shows the relative integrated “cargo” (GFP) expression intensity of the edited cells.

### Example 7: Rescue of G6PD Knock-Out Through Targeted Integration

The knock-in integration and selection approach described in Example 2 was used to target the G6PD gene in iPSCs. While iPSCs were tested for the purposes of this experiment, the described methods could be applied to other cell types. The G6PD gene encodes Glucose-6-Phosphate Dehydrogenase. This metabolic enzyme plays a key role in glycolysis and NADPH production. An AsCpf1 (AsCas12a) guide RNA that targets terminal exons of the G6PD gene is shown in Table 16 below.

TABLE 16
Guide RNA sequences

| | | gRNA targeting domain | | |
|---|---|---|---|---|
| Name | Target Site | sequence (RNA) | Location | Plasmid |
| G6PD-1 | RSQ33508 | CAGUAUGAGGGCACCUACAAG | Exon 13 | PLA1618 |
| | (SEQ ID NO: 154) | | | |

RSQ33508 (SEQ ID NO: 154) was determined to be highly specific to G6PD and has minimal off-target sites in the genome (data not shown). The G6PD gene was thus considered a good candidate gene target for the cargo integration and selection methods described herein at least in part because there are gRNAs available that are capable of specifically targeting a terminal exon (exon 13).

The gRNA RSQ33508 (SEQ ID NO: 154) was then tested to determine whether it could be used to knock-in a cassette comprising a portion of G6PD and a cargo sequence encoding GFP into a terminal exon of the G6PD locus of cells, in the process rescuing the lethal phenotype that would otherwise result by introducing RNP-induced indels into the coding region of this essential gene at a high frequency. Specifically, iPSCs were contacted with an RNP containing AsCas12a (SEQ ID NO: 62), and RSQ33508 (SEQ ID NO: 154) along with a double stranded DNA donor template (dsDNA plasmid) designed to mediate HDR at the gRNA target binding site. The double stranded DNA donor templates included a knock-in cassette with a coding sequence for GFP (“Cargo”) in frame with and downstream (3′) of a codon optimized version of the final G6PD) exon coding sequence (exon 13) and a sequence encoding the P2A self-cleaving peptide (“P2A”), similar to the dsDNA plasmid described in Example 2 for GAPDH. The G6PD) sequence in the double stranded DNA donor templates (PLA1618; comprising donor template SEQ ID NO: 51) was codon optimized to prevent further binding by the accompanying guide RNA molecule (RSQ33508). The knock-in cassette also included 3′ UTR and poly A signal sequences downstream of the Cargo sequence. An RNP containing RSQ33508 (SEQ ID NO: 154) was administered with PLA1618 (comprising donor template SEQ ID NO: 51). The dsDNA plasmid (PLA) contained a donor template with homology arms and a knock-in cassette designed to specifically encompass and render ineffective the accompanying gRNA target site following integration.

Flow cytometry was performed 7 days following nucleofection and was used to help determine to what extent the plasmid based knock-in cassette was integrated successfully at its G6PD) target site. FIG. 11A shows that cells nucleofected with RNPs containing RSQ33508 (SEQ ID NO: 154) exhibited GFP expression in approximately 10% of assayed cells, suggesting that the GFP-encoding knock-in cassette integrated at relatively low levels within these cells. FIG. 11C shows the relative integrated “cargo” (GFP) expression intensity of the edited cells.

### Example 8: Rescue of KIF11 Knock-Out Through Targeted Integration

The knock-in integration and selection approach described in Example 2 was used to target the KIF11 gene in iPSCs. While iPSCs were tested for the purposes of this experiment, the described methods could be applied to other cell types. The KIF11 gene encodes Kinesin Family Member 11. This enzyme plays a key role in vesicle movement along intracellular microtubules and chromosome positioning during mitosis. AsCpf1 (AsCas12a) guide RNAs that target terminal exons of the KIF11 gene are shown in Table 17 below.

TABLE 17
Guide RNA sequences

| | | gRNA targeting domain | | |
|---|---|---|---|---|
| Name | Target Site | sequence (RNA) | Location | Plasmid |
| KIF11-1 | RSQ33509 | CCGCCUUAAAUCCACAGCAUA | Intron 21/ | PLA1629 |
| | (SEQ ID NO: 155) | | Exon 22 | |
| KIF11-2 | RSQ33510 | UAACCAAGUGCUCUGUAGUUU | Exon 22 | PLA1630 |
| | (SEQ ID NO: 156) | | | |
| KIF11-3 | RSQ33511 | GACCUCUCCAGUGUGUUAAUG | Exon 22 | PLA1631 |
| | (SEQ ID NO: 157) | | | |

RSQ33509, RSQ33510, and RSQ33511 (SEQ ID NOs: 155-157) were each determined to be highly specific to KIF11 and have minimal off-target sites in the genome (data not shown). The KIF11 gene was thus considered a good candidate gene target for the cargo integration and selection methods described herein at least in part because there are gRNAs available that are capable of very specifically targeting a terminal exon available (exon 22). However, for any of these gRNAs to be highly suitable for the methods described herein, they need to be highly effective at introducing indels at a location in the KIF11 locus that would knock out or severely reduce gene function.

Each of these gRNAs was then tested to determine whether it could be used to knock-in a cassette comprising a portion of KIF11 and a cargo sequence encoding GFP into a terminal exon of the KIF11 locus of cells, in the process rescuing the lethal phenotype that would otherwise result by introducing RNP-induced indels into the coding region of this essential gene at a high frequency. Specifically, iPSC cells were contacted with an RNP containing AsCas12a (SEQ ID NO: 62), and RSQ33509, RSQ33510, or RSQ33511 (SEQ ID NOs: 155-157), along with a double stranded DNA donor template (dsDNA plasmid) designed to mediate HDR at each respective gRNA target binding site. The double stranded DNA donor templates included a knock-in cassette with a coding sequence for GFP (“Cargo”) in frame with and downstream (3′) of a codon optimized version of the final KIF11 exon coding sequence (exon 22) and a sequence encoding the P2A self-cleaving peptide (“P2A”), similar to the dsDNA plasmid described in Example 2 for GAPDH. The KIF11 sequence in the double stranded DNA donor templates (PLA1629, PLA1630, or PLA1631; comprising donor template SEQ ID NOs: 55-57) was codon optimized to prevent further binding by the accompanying guide RNA molecule (RSQ33509, RSQ33510, or RSQ33511; SEQ ID NOs: 155-157). The knock-in cassette also included 3′ UTR and polyA signal sequences downstream of the Cargo sequence. An RNP containing RSQ33509 (SEQ ID NO: 155) was administered with the PLA1629 plasmid (comprising donor template SEQ ID NO: 55); RSQ33510 (SEQ ID NO: 156) was administered with PLA1630 (comprising donor template SEQ ID NO: 56); and RSQ33511 (SEQ ID NO: 157) was administered with PLA1631 (comprising donor template SEQ ID NO: 57). Each particular dsDNA plasmid (PLA) contained a donor template with homology arms and a knock-in cassette designed to specifically encompass and render ineffective the particular gRNA target site following integration.

Flow cytometry was performed 7 days following nucleofection and was used to help determine to what extent each plasmid knock-in cassette was integrated successfully at its respective KIF11 target site. FIG. 11A shows that cells nucleofected with RNPs containing RSQ33509 (SEQ ID NO: 155) exhibited the greatest amount of GFP expression relative to cells nucleofected with the other RNPs targeting KIF11, suggesting that the GFP-encoding knock-in cassette integrated successfully in many of these cells. Cells nucleofected with RNPs containing RSQ33510 or RSQ33511 (SEQ ID NO: 156 or 157) also exhibited some GFP expression (FIG. 11A). FIG. 11B shows that use of the RNPs containing RSQ33509 (SEQ ID NO: 155) resulted in about 40% editing at 48 hours following transfection (the lower level possibly a result of significant cell death in the cell population at this time), correlating with the GFP expression levels depicted in FIG. 11A. Interestingly, FIG. 11B shows that use of RNPs containing RSQ33510 (SEQ ID NO: 156) resulted in about 90% observed editing rates, while RNPs containing RSQ33511 (SEQ ID NO: 157) resulted in about 65% observed editing rates, yet the GFP expression in cells transfected with these guides was relatively low when compared to RSQ33509 (SEQ ID NO: 155) transfected cells. These results suggest that the RSQ33510 or RSQ33511 (SEQ ID NO: 156 or 157) guides may not have been generating sufficiently deleterious indels in KIF11, allowing a high proportion of cells to be viable despite high editing efficiencies, such that transfected cells were not dying in large enough numbers to allow for effective selection of transfected cells with successful cargo knocked in. Thus, although the RSQ33510 and RSQ33511 (SEQ ID NO: 156 or 157) gRNAs are highly specific for their KIF11 target sites (with minimal off-targets) and exhibit high editing levels, they may still not be suitable gRNAs for the selection mechanisms described herein as they may not induce toxic indels that result in sufficient malfunction of KIF11, which in turn would lead to cell death if homologous recombination of a rescue knock-in cassette does not occur. The percentage editing was measured two days following transfection and was determined by ICE assays (as described in Hsiau et al., August 2019). FIG. 11C and FIG. 11D (representing an additional experiment where RSQ33509 was again used for editing at the KIF11 locus) show the relative integrated “cargo” (GFP) expression intensity of the edited cells.

### Example 9: Knock-In of Cargo at Essential Gene Loci Using a Viral Vector

The present example describes use of the gene editing methods described herein comprising viral vector transduction of a cell population.

The target cells described herein are collected from a donor subject or a subject in need to therapy (e.g., a patient). Following an appropriate sorting, culturing, and/or differentiation process, target cells are transduced with at least one AAV vector comprising a nucleotide sequence comprising a gRNA, a suitable nuclease, and/or a suitable rescue construct. Cells are sorted using flow cytometry to determine successful transduction, editing, integration, and/or expression events.

A population of hematopoietic stem cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of hematopoietic stem cells are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, integration, and/or expression events are determined using flow cytometry, as described herein. Following AAV transduction, a large proportion of the cells are edited at the TBP locus by the RNP and have integrated the knock-in cassette via HDR.

A population of T cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of T cells are transduced with an AAV vector (e.g., AAV6) comprising TBP targeting RNP (including Cas12a of SEQ ID NO: 62 and gRNA RSQ33503 of SEQ ID NO: 149) and PLA 1616 (comprising donor template SEQ ID NO: 49). Successful transduction, editing, integration, and/or expression events are determined using flow cytometry, as described herein. Following AAV transduction, a large proportion of the cells are edited at the TBP locus by the RNP and have integrated the knock-in cassette via HDR.

A population of NK cells are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of NK cells are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

A population of tumor-infiltrating lymphocytes (TILs) are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of tumor-infiltrating lymphocytes (TILs) are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

A population of neurons are transduced with an AAV vector (e.g., AAV6) comprising GAPDH targeting RNP (including Cas12a of SEQ ID NO: 62 and gRNA RSQ22337 of SEQ ID NO: 95) and PLA1593 (comprising donor template SEQ ID NO: 44). Successful transduction, editing, knock-in cassette integration, and/or expression events are determined using flow cytometry, as described herein. Following AAV transduction, a large proportion of the cells are edited at the GAPDH locus by the RNP and have integrated the knock-in cassette via HDR. A population of neurons are transduced with an AAV vector (e.g., AAV6) comprising TBP targeting RNP (including Cas12a of SEQ ID NO: 62 and gRNA RSQ33503 of SEQ ID NO: 149) and PLA1616 (comprising donor template SEQ ID NO: 49). Successful transduction, editing, knock-in cassette integration, and/or expression events are determined using flow cytometry, as described herein. Following AAV transduction, a large proportion of the cells are edited at the TBP locus by the RNP and have integrated the knock-in cassette via HDR.

### Example 10: Knock-In of Cargo at Essential Gene Loci Using a Viral Vector

The present example describes gene editing of populations of T cells using methods described herein comprising viral vector transduction of populations of T cells. The methods described herein can be applied to other cell types as well, such as other immune cells.

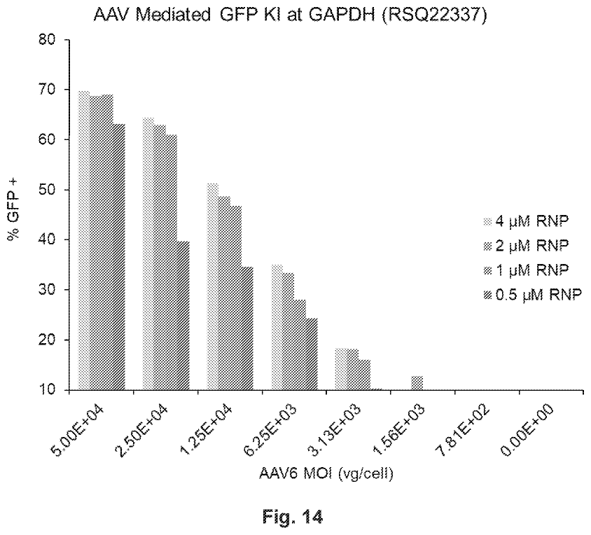

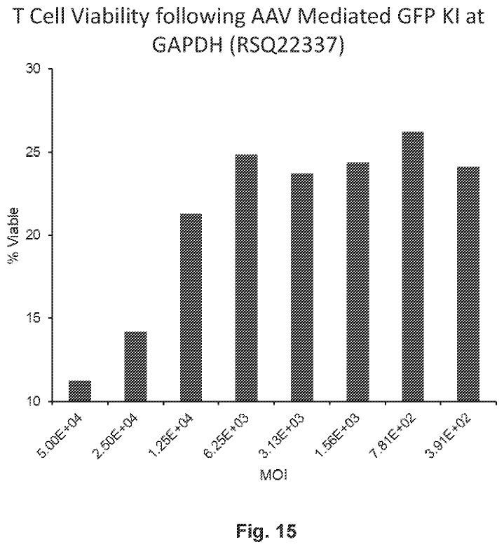

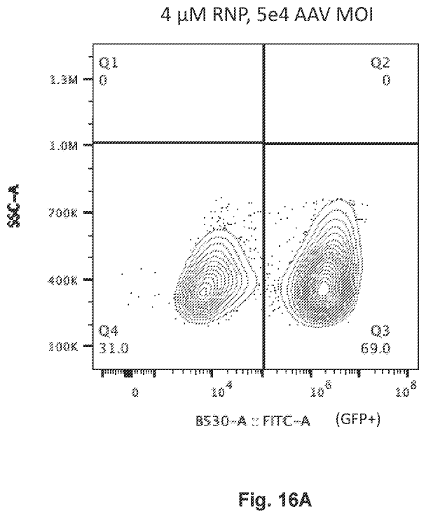

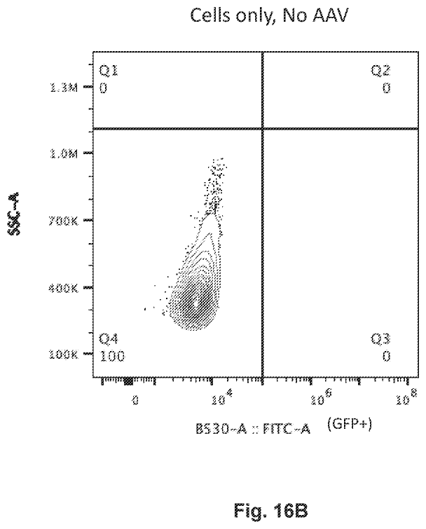

T cells were thawed in a bead bath as known in the art and were removed from the bath on day two. Cells were electroporated on day four after thawing, in brief 250,000 T cells per well in a Lonza 96-well cuvette were suspended in buffer P2 and electroporated using pulse code CA-137 with varying concentrations of RNP comprising gRNA RSQ22337 (SEQ ID NO: 95) and Cas12a (SEQ ID NO: 62) targeting the GAPDH gene (4 μM RNP, 2 μM RNP, 1 μM RNP, or 0.5 μM RNP). Appropriate media was added to cells immediately after electroporation and cells were allowed to recover for 15 minutes. AAV6 viral particles comprising a donor plasmid construct containing a knock-in cassette with a GFP cargo were then added to T cells at varying multiplicity of infection (MOI) concentrations (5E4, 2.5E4, 1.25E4, 6.25E3, 3.13E3, 1.56E3, or 7.81E2). The donor plasmid was designed as described in Example 2, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for GFP (“Cargo”), a stop codon and polyA signal sequence. T cells were split two days later, and then every 48 hours until they were analyzed by flow cytometry. T cells were sorted using flow cytometry seven days post electroporation to determine successful transduction, transformation, editing, knock-in cassette integration, and/or expression events (see FIG. 14, FIG. 15, FIG. 16A, and FIG. 16B). As shown in FIG. 14, populations of T cells were transduced with 4 μM RNP, 2 μM RNP, 1 μM RNP, or 0.5 μM RNP, at various AAV6 multiplicity of infection (MOI) (5E4, 2.5E4, 1.25E4, 6.25E3, 3.13E3, 1.56E3, or 7.81E2). High proportions of GFP integration at the GAPDH gene were observed in T cell populations transduced/transformed with all RNP concentrations at 5E4 AAV6 MOI and were observed with RNP concentrations greater than 1 μM when cells were transduced with AAV6 MOI as low as 1.25E4 (see FIGS. 14 and 16A). Control experiments with no AAV transduction resulted in T cell populations that displayed no GFP integration events (see FIG. 16B). T cell viability was measured four days after cells were transformed with RNPs and AAV6 at various MOI (FIG. 15).

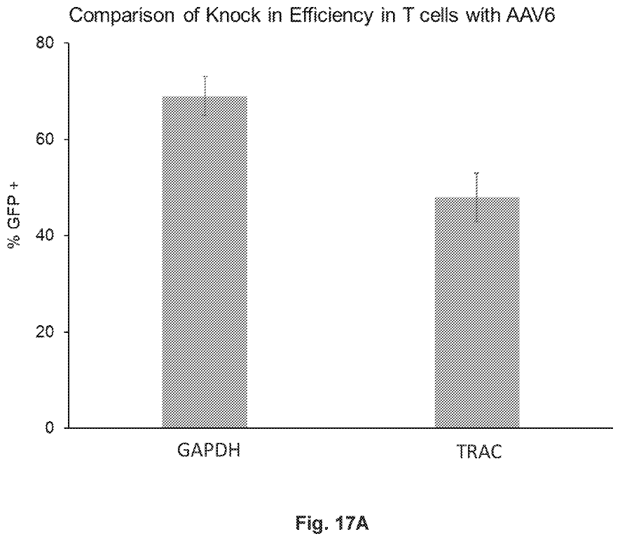

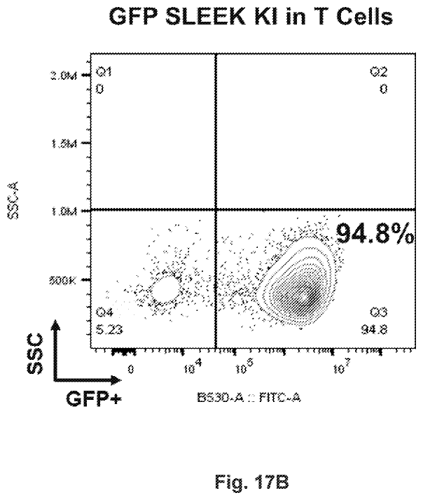

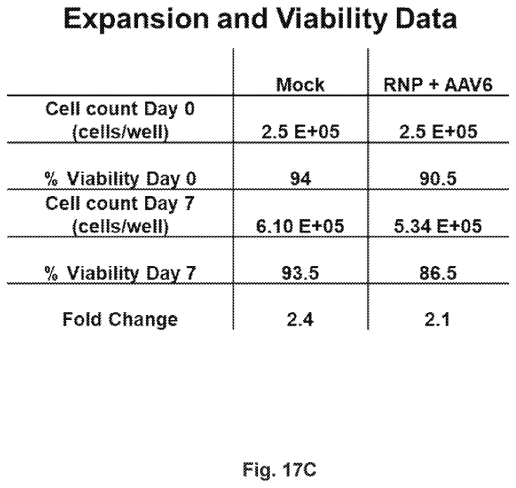

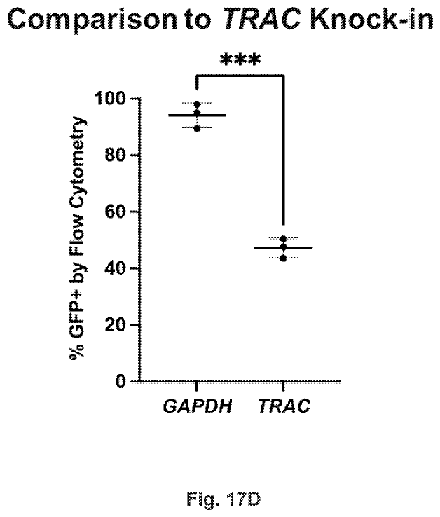

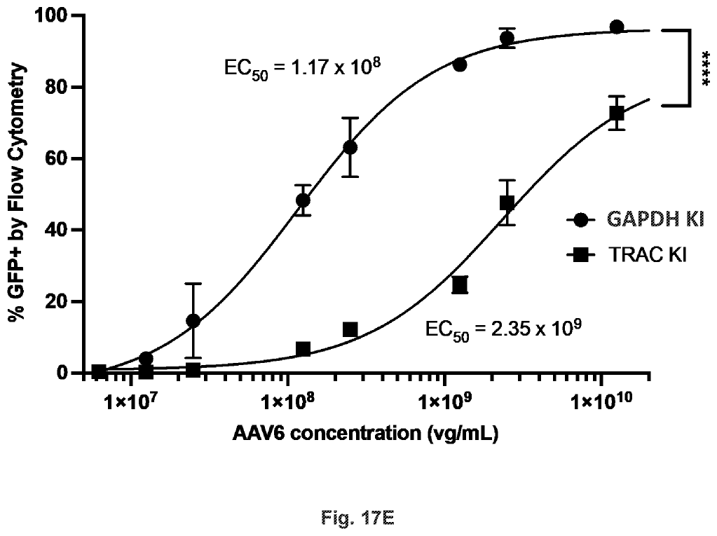

Furthermore, knock-in efficiencies using methods described herein were compared to optimized versions of methods known in the art. In brief, T cell populations were transduced with AAV6 vector comprising a donor template suitable for knock-in of GFP at the GAPDH gene as described herein, and were transformed with gRNA RSQ22337 (SEQ ID NO: 95) and Cas12a (SEQ ID NO: 62) as described above; alternatively, T cell populations were subject to highly optimized GFP knock-in at the TRAC locus using AAV6 vector transduction (see e.g., Vakulskas et al. A high-fidelity Cas9 mutant delivered as a ribonucleoprotein complex enables efficient gene editing in human hematopoietic stem and progenitor cells. Nat Med. 2018; 24(8): 1216-1224). Flow cytometry was utilized to measure knock-in efficiency (determined by percentage of T cell population expressing GFP, measured 7 days post-electroporation). Knock-in rates at the TRAC locus were high (˜50%) when compared to publicly described integration frequencies for similar methodologies, however, knock-in efficiency at the GAPDH gene using methods described herein facilitated by AAV6 transduction were significantly (p=0.0022 using unpaired t-test) higher (˜68%) (see FIG. 17A). The same RNP concentration, AAV6 MOI, and homology arm lengths were utilized in both experiments, averaged results from three independent biological replicates are shown (see FIG. 17A). Thus, the methods described herein can be used to isolate a population of modified cells, such as immune cells like T cells, that highly express a gene of interest relative to other gene knock-in methods.

### Example 11: CD16 Knock-In iPSCs Give Rise to Edited iNKs with Enhanced Function

The present example describes use of gene editing methods described herein to create modified immune cells suitable for killing cancer cells.

PSCs were edited using the exemplary system illustrated in FIGS. 3A, 3B, and 3C, and described in Example 2. In brief, the GAPDH gene was targeted in iPSCs using AsCpf1 (SEQ ID NO: 62), and a guide RNA (RSQ22337) (SEQ ID NO: 95), resulting in a double-strand break towards the 5′ end of the last exon of GAPDH (exon 9). The CRISPR/Cas nuclease and guide RNA were introduced into cells by nucleofection (electroporation) of a ribonucleoprotein (RNP) according to known methods. The cells were also contacted with a double stranded DNA donor template (dsDNA plasmid, comprising donor template SEQ ID NO: 205) that included a donor template comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for CD16 (“Cargo”) (a non-cleavable CD 16; SEQ ID NO: 165), a stop codon and polyA signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B).

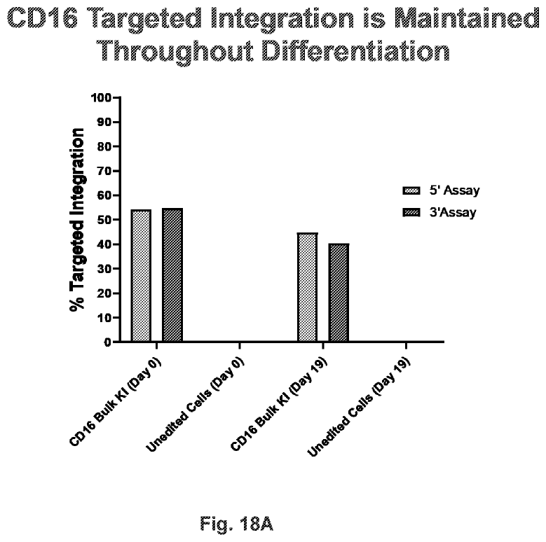

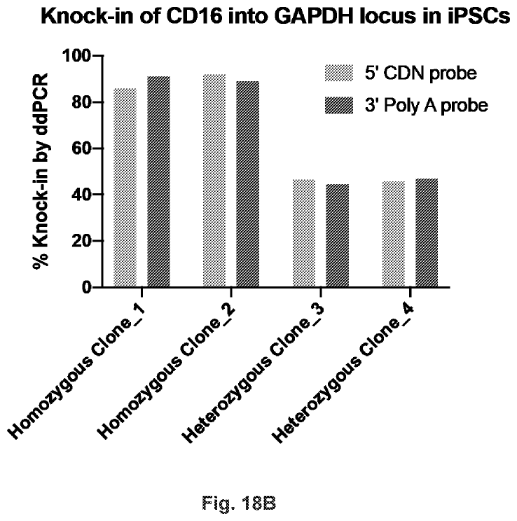

The cargo gene CD16 was successfully integrated into the GAPDH gene of iPSCs at high efficiencies using the selection systems described herein. FIG. 18A shows the efficiency of CD16-encoding “cargo” integration in the GAPDH gene at 0 days post-electroporation and at 19 days post-electroporation in iPSCs transformed with RNPs at a concentration of 4 μM and the dsDNA plasmid encoding CD16, or in “unedited cells” that were not transformed with the dsDNA plasmid. Knock-in was measured in bulk edited CD16 KI cells using ddPCR targeting the 5′ or 3′ position of the knock-in “cargo” using a primer in the 5′ of the gRNA target site or a primer in the 3′ of the site in the poly A region, increasing the reliability of the result. As shown in FIG. 18A, CD16 was stably knocked-in and present in bulk edited cell populations more than two weeks following electroporation and targeted integration of the knock-in cassette.

From bulk edited cell populations, single cells were propagated to homogenize genotypes. Shown in FIG. 18B are four edited cell populations: homozygous clone 1, homozygous clone 2, heterozygous clone 3, and heterozygous clone 4. The homozygous clones contained two alleles of the GAPDH gene that comprised CD16 knock-in, while heterozygous clones contained one allele of the GAPDH gene that comprised CD16 knock-in (measured using ddPCR of the 5′ and 3′ positions of the knock-in cargo).

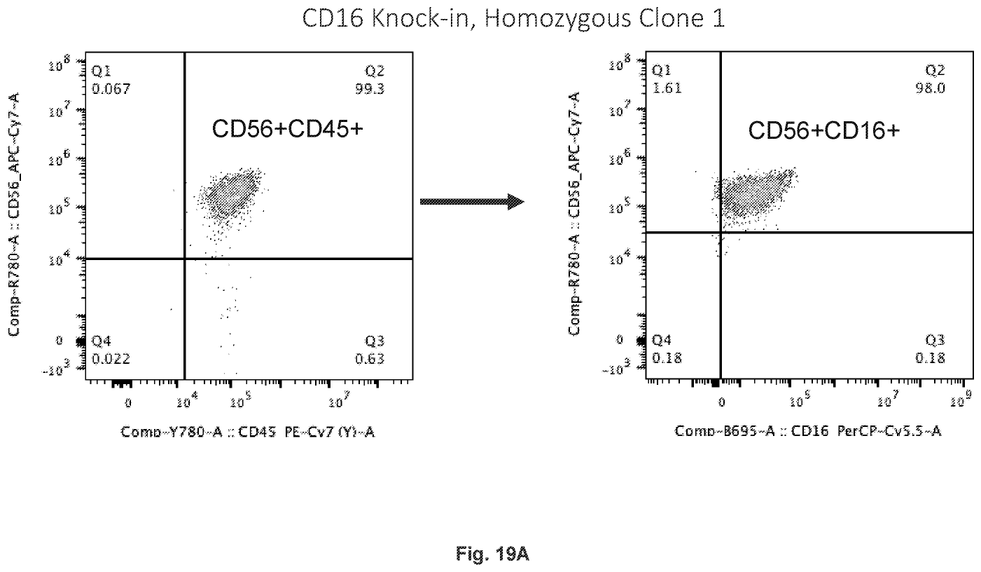

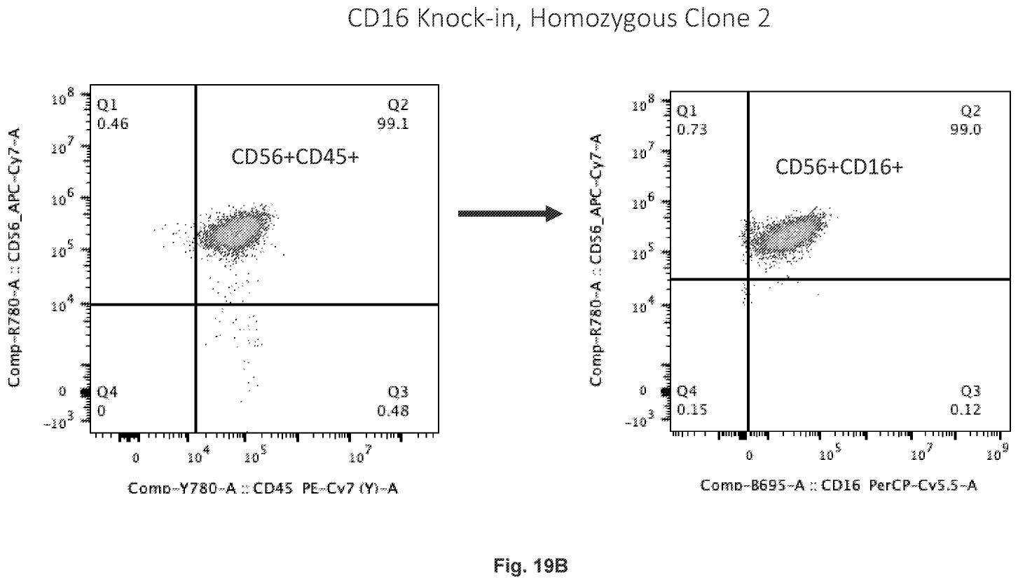

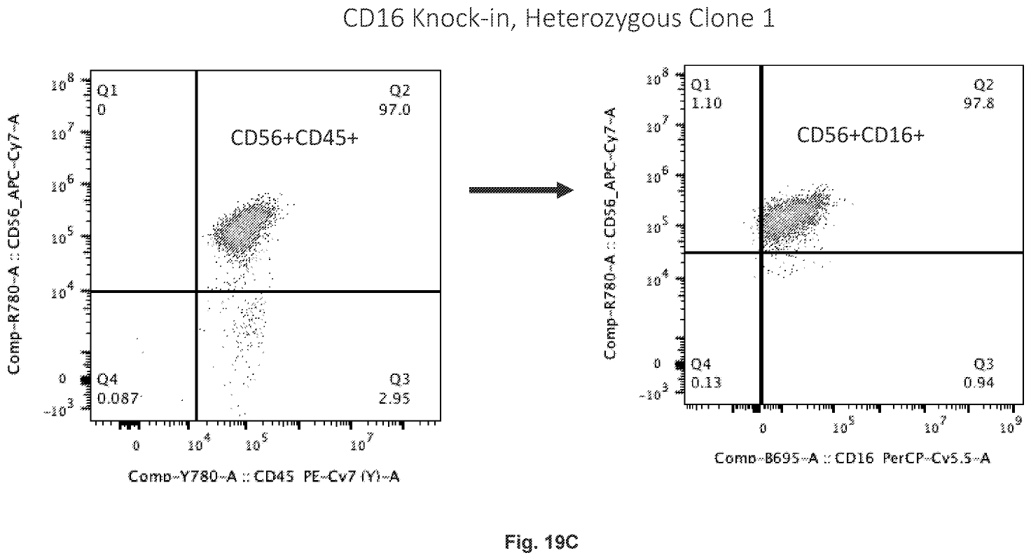

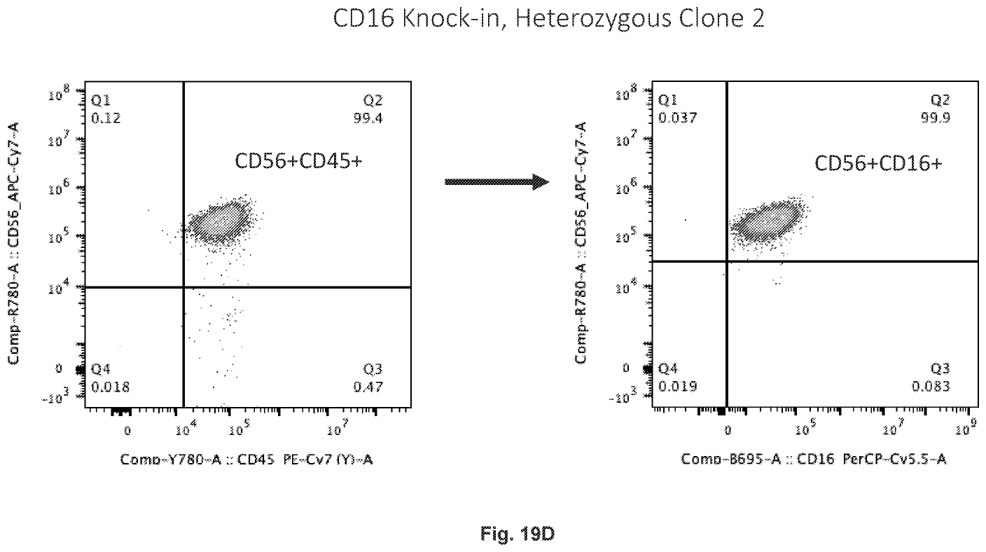

Following confirmation of CD16-encoding “cargo” integration at the GAPDH gene, homogenized cell lines were differentiated into Natural Killer (NK) immune cells using spin embryoid body methods as known in the art. In brief, iPSCs were placed in an ultra-low attachment 96-well plate at 5,000 to 6,000 cells per well in order to form embryoid bodies (EBs). On day 11 EBs were transferred to a flask where they remain for the remainder of the experiment (see Ye Li et al., Cell Stem Cell. 2018 Aug. 2; 23(2): 181-192.e5). At day 32 of the differentiation process, cells were analyzed using flow cytometry methods known in the art. Following standard control gating experiments (see Ye Li et al., Cell Stem Cell. 2018 Aug. 2; 23(2): 181-192.e5), the differentiation process was analyzed using expression of markers CD56 and CD45, following this, co-expression of markers CD56 and CD16 was measured. As shown in FIG. 19A-19D, in general, cells that were positive for CD56 expression were also positive for CD16 expression (98%, 99%, 97.8%, and 99.9% respectively), indicating that both homozygous and heterozygous TI clones had stable and robust CD16 expression levels.

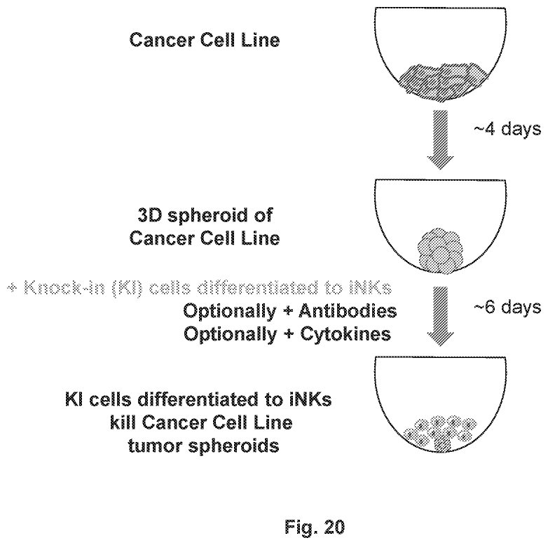

These differentiated iNK cells comprising knock-in of the gene of interest (CD16) at the GAPDH gene were then subject to challenge by various cancer cell lines to determine their cytotoxic capacity. An exemplary 3D solid tumor killing assay is depicted in FIG. 20. In brief, spheroids were formed by seeding 5,000 NucLight Red labeled SK-OV-3 cells in 96 well ultra-low attachment plates. Spheroids were incubated at 37° C. before addition of effector cells (at different E:T ratios) and any optional agents (e.g., cytokines, antibodies, etc.), spheroids were subsequently imaged every 2 hours using the Incucyte S3 system for up to 600 hours. Data shown are normalized to the red object intensity at time of effector addition. Normalization of spheroid curves maintains the same efficacy patterns observed in non-normalized data. Using this assay, the cytotoxicity of iNKs differentiated from iPSCs comprising knock-in of CD16 at the GAPDH gene was measured.

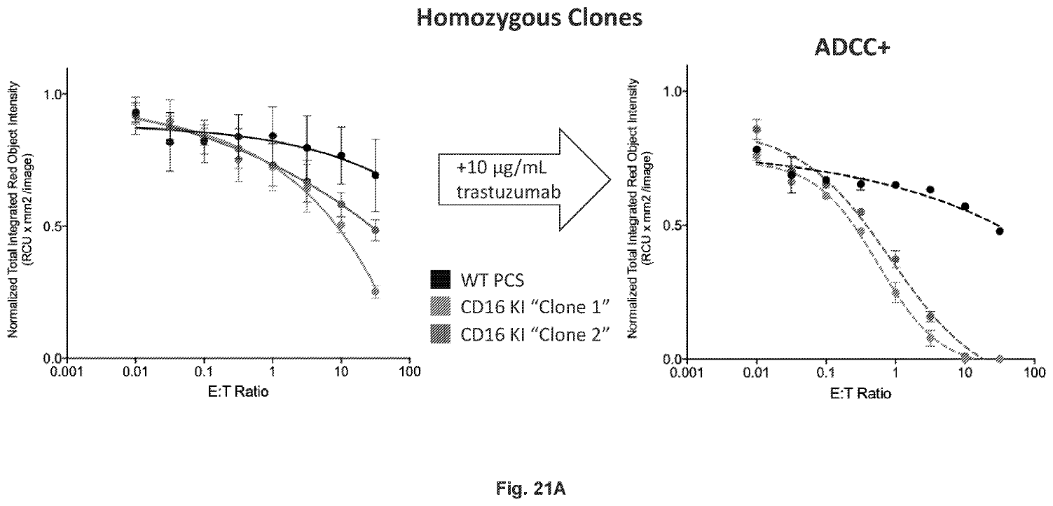

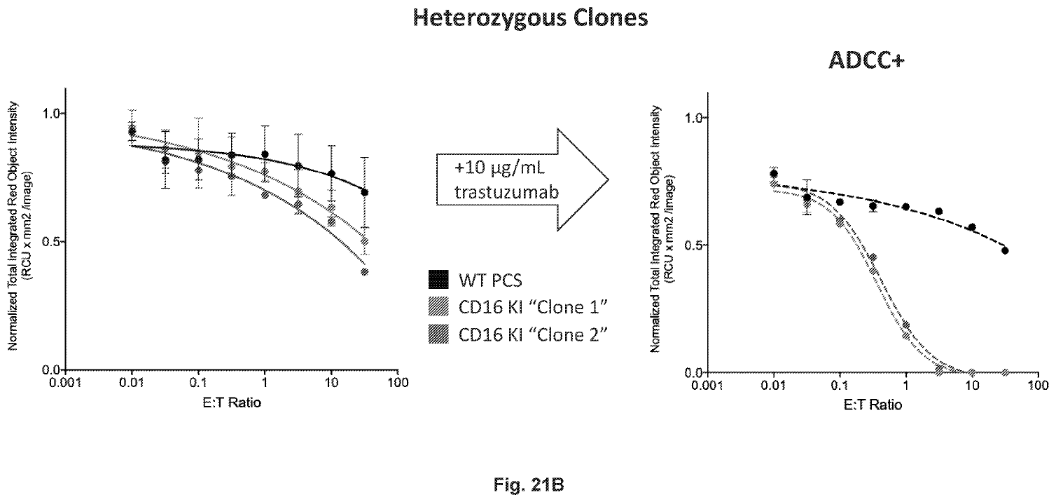

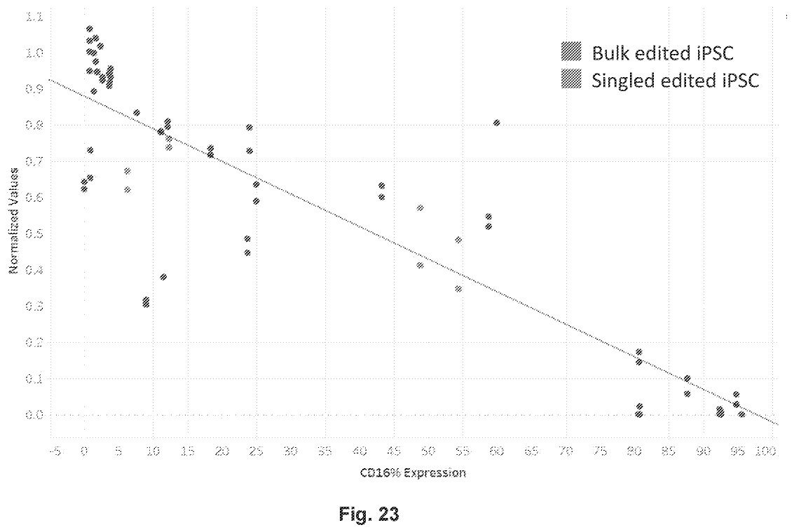

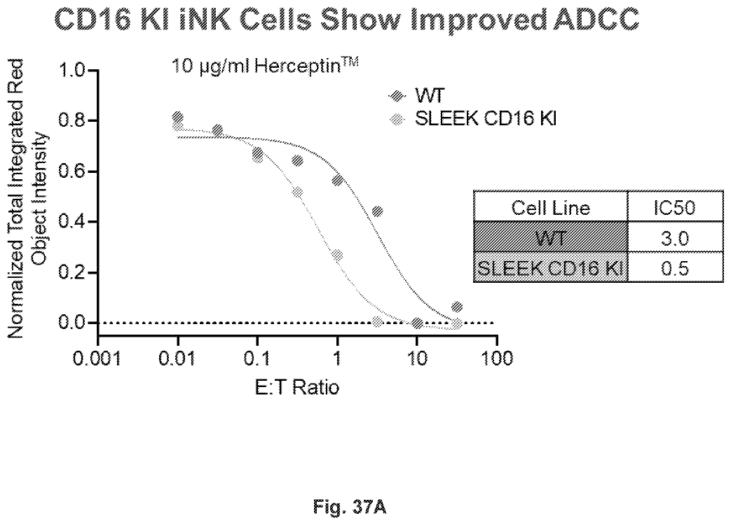

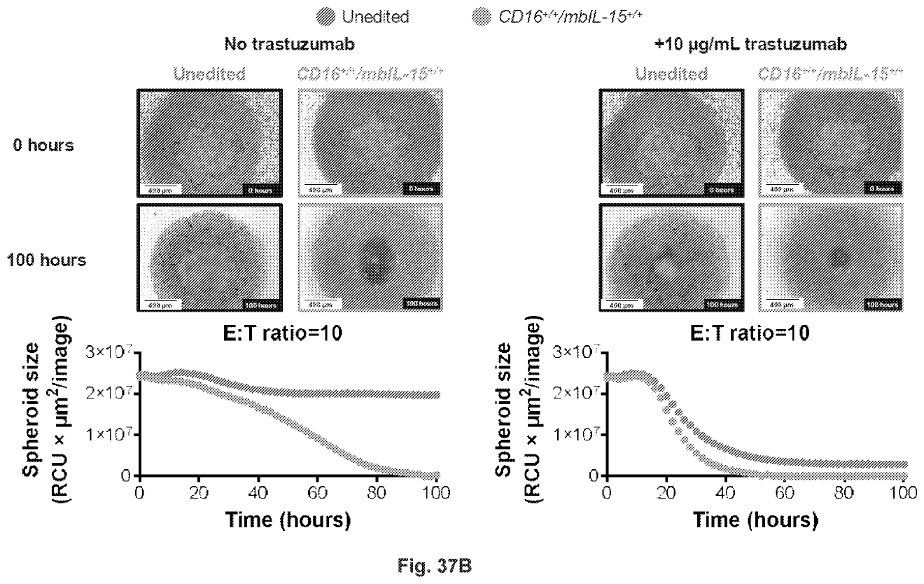

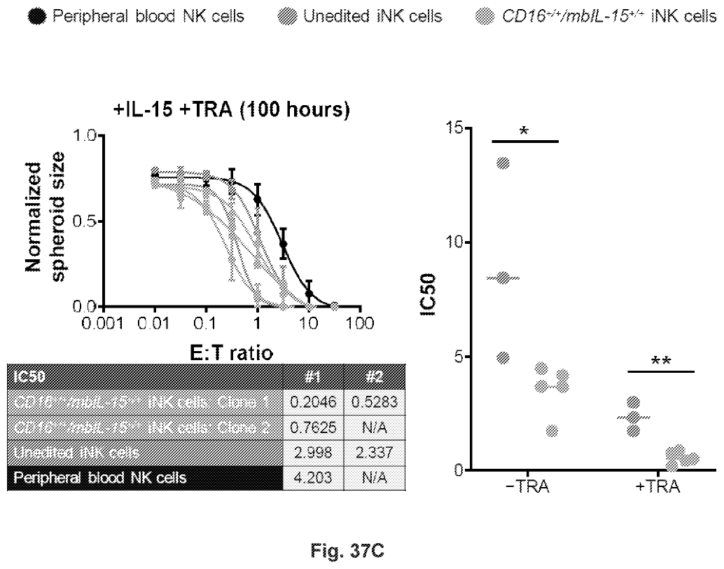

As shown in FIGS. 21A and 21B, both homozygous edited iNK lines and both heterozygous edited iNK lines comprising CD16 knocked-in at the GAPDH gene were capable of reducing the size of SK-OV-3 spheroids more effectively than unedited iNK control cells (WT PCS) or control cells with GFP knocked-in to the GAPDH gene (WT GFP KI) (averaged data from 2 assays). The edited homozygous and heterozygous iNK cells comprising CD16 at GAPDH also reduced the size of SK-OV-3 spheroids more effectively than control cells with GFP knocked-in to the GAPDH gene (data not shown). Introduction of 10 μg/mL of the antibody trastuzumab greatly enhanced the killing capacity of the CD16 KI iNKs when compared to control cells, likely as a function of increased antibody dependent cellular cytotoxicity (ADCC) due to increased FcγRIII (CD16) expression levels (see FIG. 37A). The results of a number of solid tumor killing assays were plotted against the CD16 expression levels of CD16 KI edited iNKs (derived from bulk edited iPSCs or singled edited iPSCs). At an E:T ratio of 3.16:1, there is a correlation shown between the percentage of a cell population expressing CD16, and the amount of cell killing that occurred (see FIG. 23).

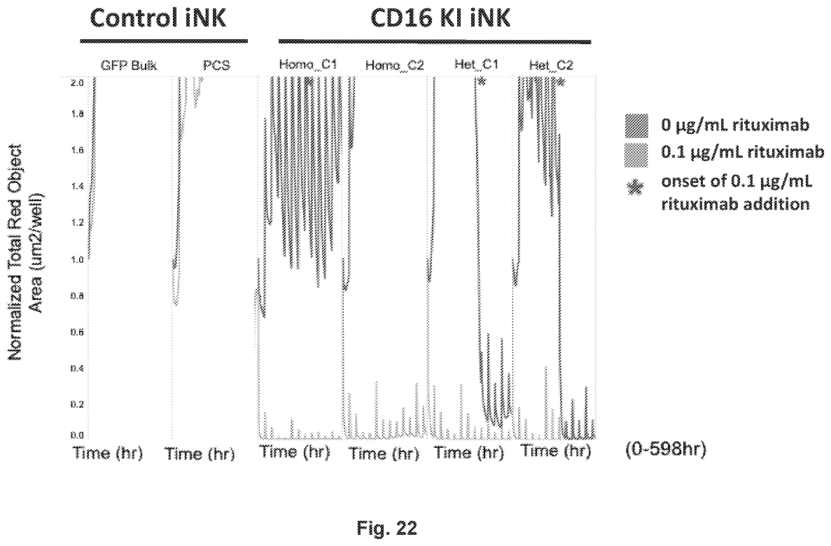

To further elucidate the functionality of the edited iNKs, the cells were subjected to repeated exposure to tumor cells, and the ability of the edited iNKs to kill tumor targets repeatedly over a multiday period was analyzed in an in vitro serial killing assay. Results of this experiment are depicted in FIG. 22. At day 0 of the assay, 10×10⁶ Raji tumor cells (a lymphoblast-like cell line of hematopoietic origin) and 2×10⁵ iNKs were plated in each well of a 96-well plate in the presence or absence of 0.1 μg/mL of the antibody rituximab. At approximately 48 hour intervals, a bolus of 5×10³ Raji tumor cells was added to re-challenge the iNK population. As shown in FIG. 22, the edited iNK cells (CD16 KI iNK heterozygous or homozygous) exhibited continued killing of Raji cells after multiple challenges with Raji tumor cells (up to 598 hours), whereas unedited iNK cells were limited in their serial killing effect. The data show that iNK cells comprising homozygous or heterozygous CD16 KI at GAPDH results in prolonged and enhanced tumor cell killing. Furthermore, the efficacy of heterozygous CD16 KI iNKs highlights the potential for biallelic insertion of two different knock-in cassettes, e.g., comprising CD16 in one allele and a different gene of interest in the other allele of a suitable essential gene (e.g., GAPDH, TBP, KIF11, etc.).

### Example 12: Knock-In of Immunologically Relevant Sequences at a Suitable Essential Gene Locus (Monocistronic or Bicistronic)

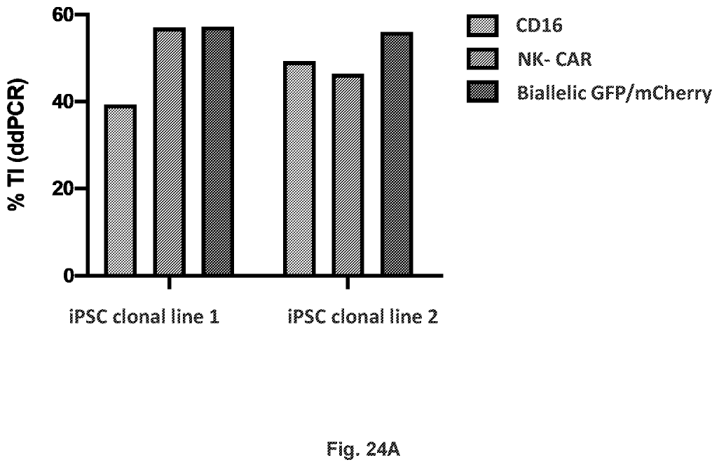

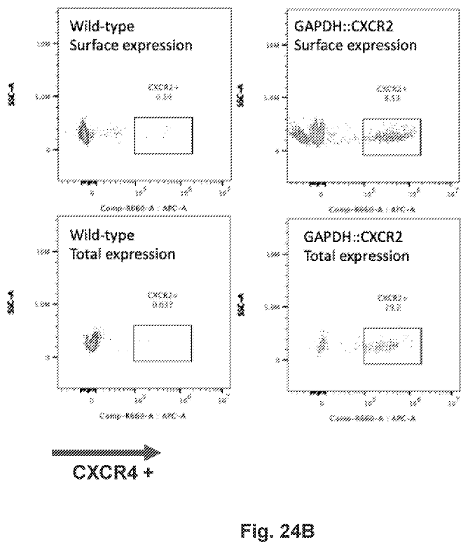

Positive targeted integration events at the GAPDH gene and cellular phenotypes were noted for integration of GFP, CD47, or CD16 as described above in Example 2 and Example 11. Additional or alternative cargo sequences may be incorporated into the GAPDH gene or other suitable essential genes as described herein with high integration rates. The essential gene GAPDH was targeted in iPSC cells using an RNP containing AsCpf1 (SEQ ID NO: 62) and a guide RNA (RSQ22337; SEQ ID NO: 95), resulting in a double-strand break towards the 5′ end of the last exon of GAPDH (exon 9), as described in Example 2. A donor plasmid containing a knock-in cassette with the cargo of interest was also electroporated with the RNP. As shown in FIG. 24A, the targeted integration (TI) rates at the GAPDH gene for cargos such as a) CD16, b) a CAR suitable for expression in NK cells, or c) biallelic GFP/mCherry, were all greater than 40% when assayed in two independent iPSC clonal lines when measured using ddPCR. As shown in FIG. 24B, the targeted TI rates at the GAPDH gene for a CXCR2 cargo was at least 29.2% of bulk edited iPSCs (expression determined using flow cytometry), while surface expression of CXCR2 was observed in approximately 8.5% of the bulk edited iPSCs (expression determined using flow cytometry). By contrast, unedited iPSCs very small amounts of CXCR2 (approximately 1%) by flow cytometry (data not shown).

An exemplary ddPCR experiment was used to measure the targeted integration (TI) rates as follows. In brief, TI was measured using a universal set of primers that captures both the 5′ homology arm and 3′ poly A tail for the GAPDH terminal exon region, and can detect cargos independent of the particular sequence of the specific cargo. The 5′ CDN primer and 3′ PolyA primer and FAM fluorophore probes are made in combination. An appropriate reference gene probe is a TTC5 HEX probe. For the reaction, probes, genomic DNA, BioRad master mix, and 2× control buffer were mixed together in ratios consistent with manufacturer recommendations. First, genomic DNA was placed in the BioRad 96 well plate (9.2 μl total genomic DNA+water), next, master mix with primer probes sets (13.8 μl per well) were added. Water controls comprised a 5′ primer probe set master mix in one well, and a 3′ primer probe set master mix in a different well. For blank well controls, a 50/50 mix of 2× control buffer and water (25 μl total) was added. The auto droplet generator was then prepared and run. Once droplets were generated, the ddPCR plates were sealed at 180° C. and then placed in a thermocycler for amplification. 5′ CDN primer: CATCGCATTGTCTGAGTAGGTGTC (SEQ ID NO: 219), 3′ PolyA primer: TGCCCACAGAATAGCTTCTTCC (SEQ ID NO: 220), FAM probe: TCCCCTCCTCACAGTTGCCA (SEQ ID NO: 221), TTC5 reference gene forward primer: GGAGAAAGTGTCCAGGCATAAG (SEQ ID NO: 222), TTC5 reference gene reverse primer: CTCCATCCCACTATGACCATTC, (SEQ ID NO: 223), TTC5 FAM probe: AGTTTGTGTCAGGATGGGTGGT (SEQ ID NO: 224).

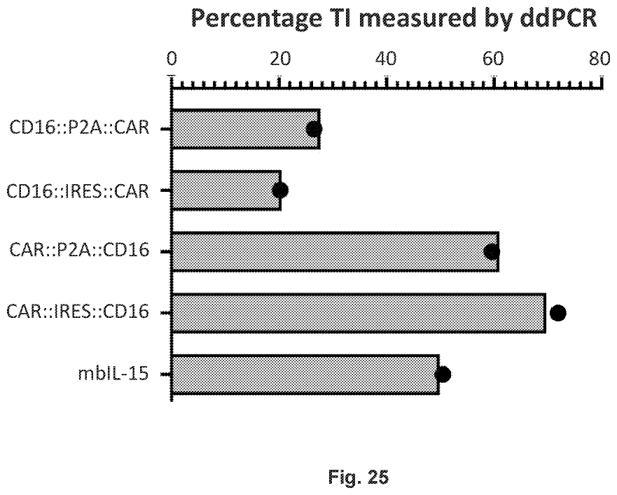

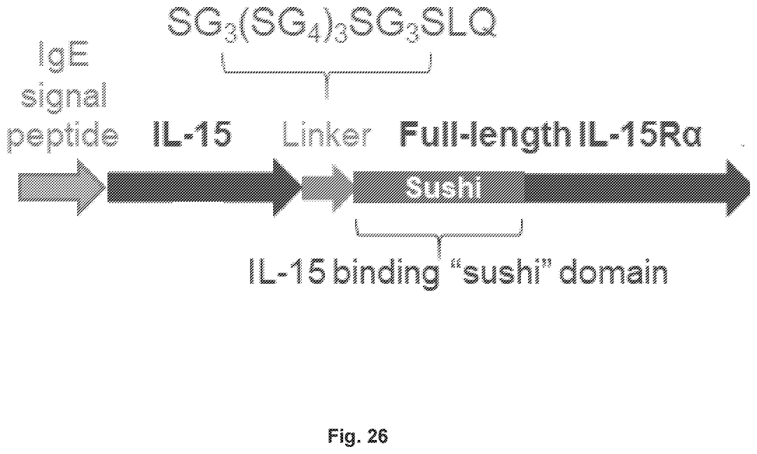

Next, the cargo integration and selection methods described herein were tested using a number of bicistronic knock-in cassettes that contained CD16 and an NK suitable CAR in different 5′-to-3′ orders (e.g., CD16 followed by the CAR, or the CAR followed by CD16) and separated by a P2A or IRES sequence. The essential gene GAPDH was targeted in iPSC cells using an RNP containing AsCpf1 (AsCas12a, (SEQ ID NO: 62)) and a guide RNA (RSQ22337; SEQ ID NO: 95), resulting in a double-strand break towards the 5′ end of the last exon of GAPDH (exon 9), as described in Example 2. A donor plasmid containing each of the knock-in cassettes depicted in FIG. 25 was also electroporated with the RNP. As shown in FIG. 25, the TI rates for the bicistronic constructs comprising CD16 and the NK suitable CAR ranged from 20-70% when measured in the bulk edited cells using ddPCR at day 0 post-transformation. In addition, a membrane bound IL-15 (mbIL-15) cargo gene (a fusion comprising IL-15 linked to a Sushi domain and a full-length IL-15Rα, as depicted in FIG. 26) was also knocked into the GAPDH locus using RNPs comprising (RSQ22337) and Cas12a at a concentration of 4 μM and the dsDNA plasmid encoding mbIL-15 at 5 μg (PLA1632; comprising donor template SEQ ID NO: 45) to determine if additional genes of interest could be integrated into an essential gene at high levels within a population of edited cells. FIG. 25 shows that the mbIL-15 cargo was knocked into the GAPDH locus at a percentage TI of greater than 50% as measured by ddPCR (day 0 post-transformation). Thus, the methods described herein can be used to isolate populations of edited cells, such as iPSCs, that have very high levels of a gene of interest knocked into an essential gene locus, such as GAPDH.

### Example 13: IL-15 and/or IL-15/IL15-Rα Knock-In iPSCs Give Rise to Edited iNKs with Enhanced Function

The present example describes use of gene editing methods described herein to create modified immune cells suitable for cancer cell killing.

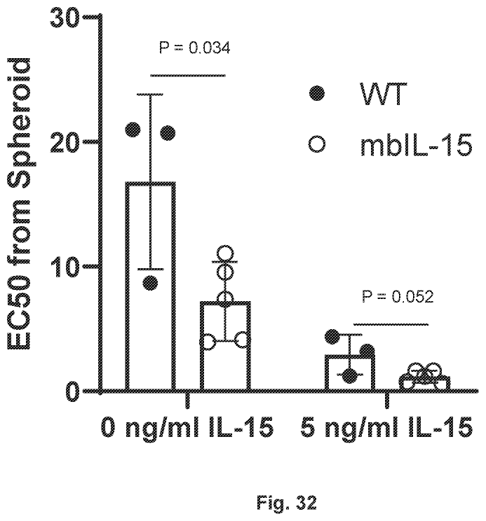

PSCs were edited using the exemplary system illustrated in FIGS. 3A, 3B, and 3C, and described in Example 2. In brief, the GAPDH gene was targeted in iPSCs using RNPs containing AsCpf1 (AsCas12a, SEQ ID NO: 62), and a guide RNA (RSQ22337; SEQ ID NO: 95), resulting in a double-strand break towards the 5′ end of the last exon of GAPDH (exon 9). The CRISPR/Cas nuclease and guide RNA were introduced into cells by nucleofection (electroporation) of a ribonucleoprotein (RNP) according to known methods. The cells were also contacted with a double stranded DNA donor template (dsDNA plasmid) that included a donor template comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence for mbIL-15 as shown in FIG. 32 (“Cargo”) (SEQ ID NO: 172), a stop codon and poly A signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B). The 5′ and 3′ homology arms flanking the cargo coding sequence of the donor template were designed to correspond to sequences located on either side of the endogenous stop codon in the genome of the cell.

The cargo gene mbIL-15 (as shown in FIG. 26) was successfully integrated into the GAPDH gene of iPSCs at high efficiencies using the selection systems described herein (see Example 12). FIG. 25 shows the efficiency of the mbIL-15-encoding “cargo” in GAPDH at 0 days post-electroporation in iPSCs transformed with RNPs comprising (RSQ22337) and Cas12a at a concentration of 4 μM and the dsDNA plasmid encoding mbIL-15 at 5 μg (PLA1632; comprising donor template SEQ ID NO: 45). Genomic DNA was extracted approximately seven days post nucleofection. After genomic DNA extraction ddPCR was performed.

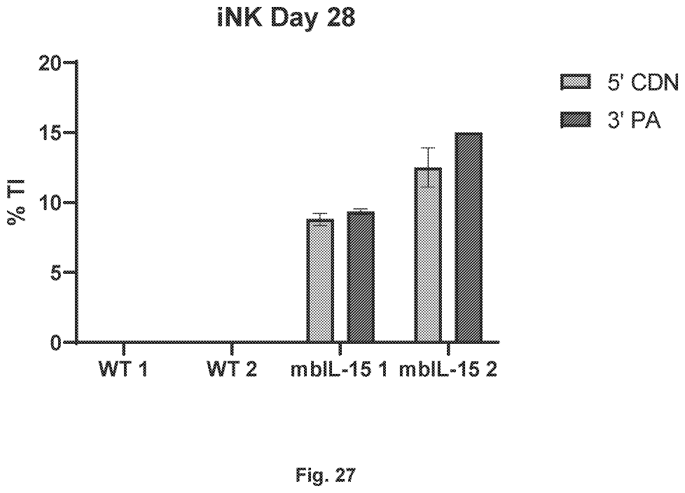

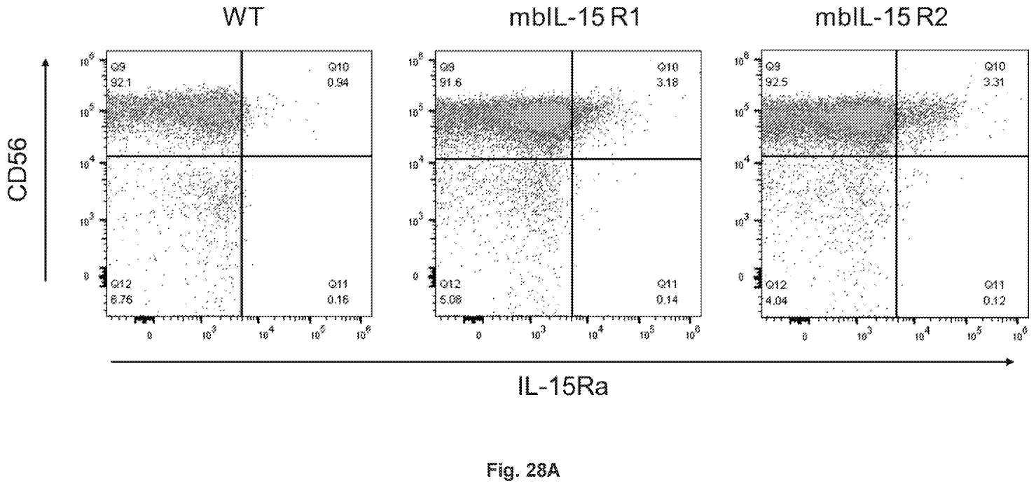

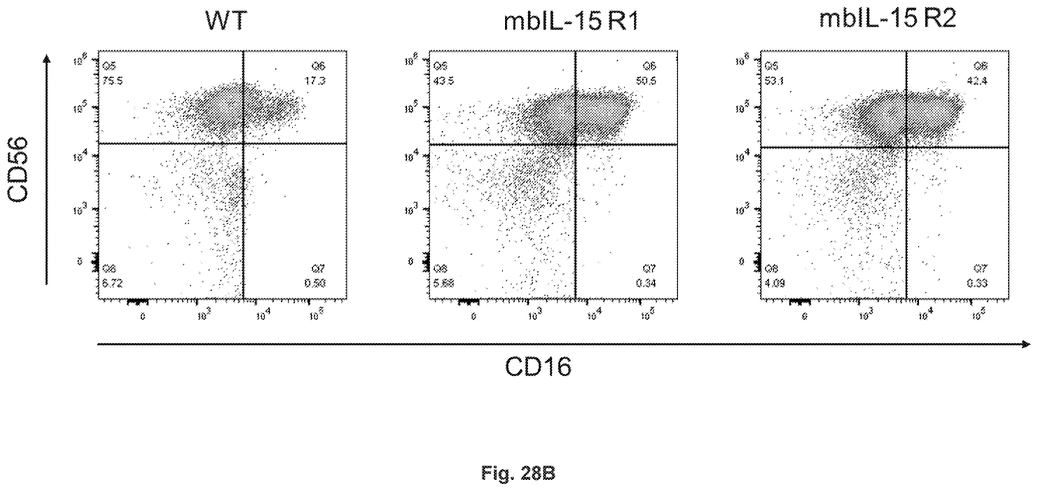

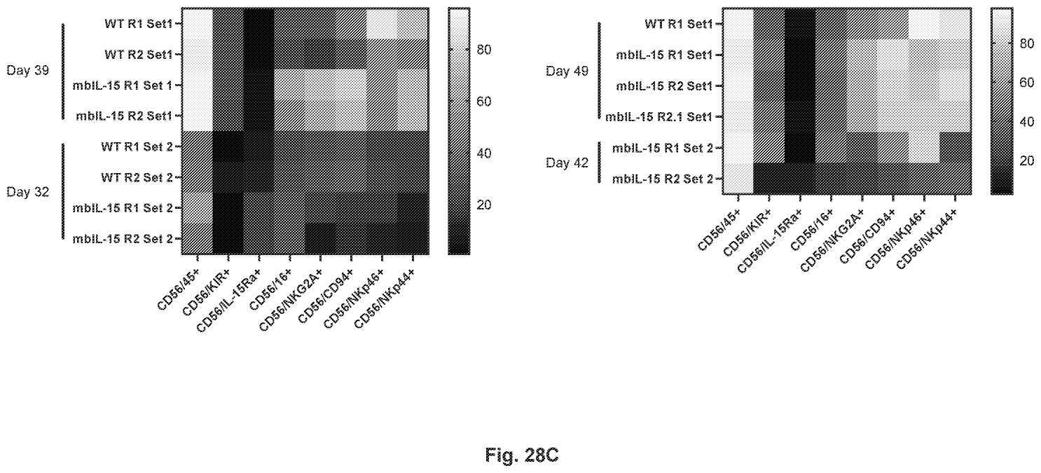

Two separate populations of the bulk edited mbIL-15 KI iPSC cells were then differentiated into iNK cells and the TI rates were measured using ddPCR at day 28 of the iNK differentiation process. FIG. 27 shows that TI integrate rates for these edited iNK cell populations ranged from 10-15%. While the TI rates in the iNK populations decreased when compared to the TI at day 0 post-electroporation of iPSCs, the TI integration levels within these cell populations remained significant. At day 32 post-differentiation initiation, flow cytometry was conducted to determine the proportion of cells expressing CD56 and exogenous IL-15Rα in these edited iNK cell populations (see FIG. 28A). The CD56 and CD16 co-expression levels were also determined in these edited iNK cell populations (see FIG. 28B). The bulk edited mbIL-15 KI cell populations were also analyzed for markers of differentiation by flow cytometry on day 32, day 39, day 42, and day 49 post-differentiation initiation (see FIG. 28C).

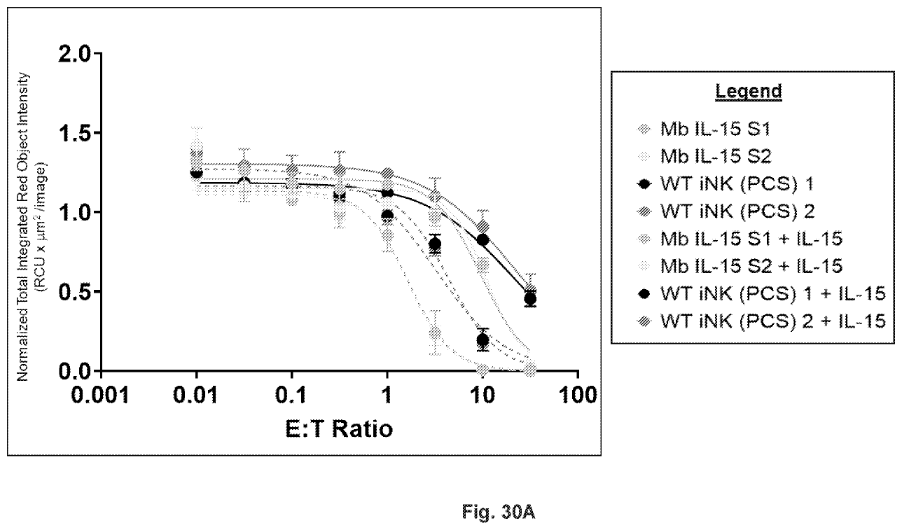

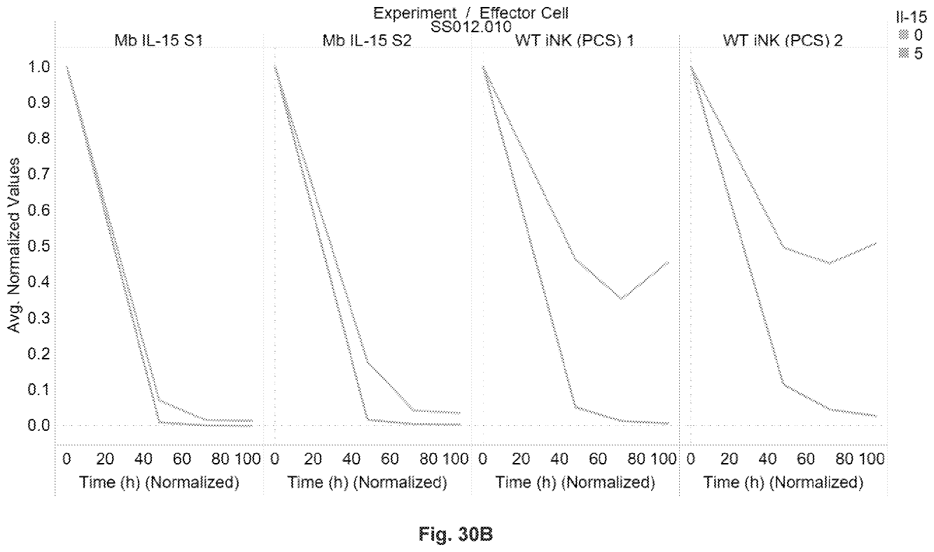

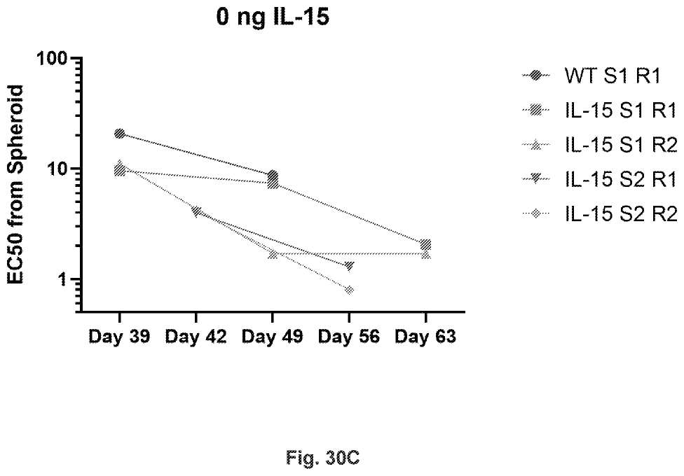

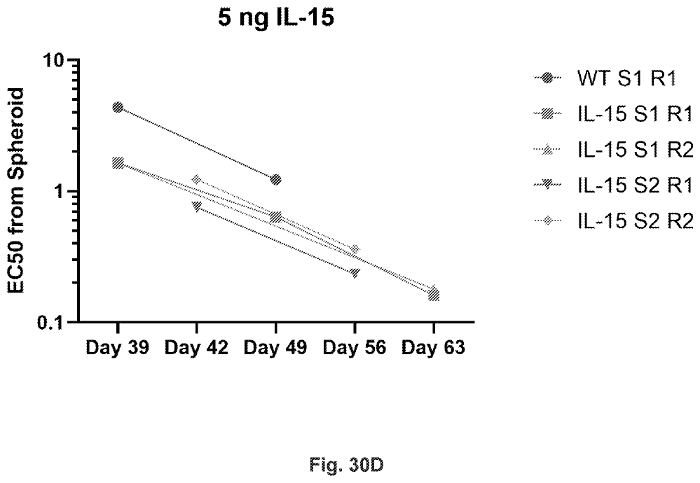

At day 39 following the initiation of differentiation from the edited iPSCs into iNKs, cells were challenged in 3D spheroid killing assays as described in Example 11 and depicted in FIG. 20. Using this assay, the cytotoxicity of iNKs differentiated from iPSCs comprising knock-in of mbIL-15 at the GAPDH gene was measured (see FIG. 30A). Cells were tested in the presence or absence of 5 ng/ml exogenous IL-15. As shown in Table 18 and FIG. 30A, mbIL-15 KI iNK cells (Mb IL-15 S1 and Mb IL-15 S2 populations) exhibited more efficient tumor cell killing when compared to unedited parental cells differentiated into iNKs (“WT” PCS, 1 and 2). Of note, mbIL-15 KI iNK cells exhibited better tumor cell killing in the absence of exogenous IL-15 relative to WT iNK cells in the absence of endogenous IL-15 at lower E:T ratios. The mbIL-15 KI iNK cells also exhibited better tumor cell killing in the presence of low concentrations of exogenous IL-15 (5 ng/mL) when compared to unedited WT iNK cells in the presence of the same concentration of exogenous IL-15. In addition, at higher E:T ratios, mbIL-15 KI iNKs outperformed WT iNKs without the addition of exogenous IL-15 (see FIG. 30B). The above described 3D spheroid killing assay was repeated on mbIL-15 KI iNKs and control cells on day 42 and day 49, and for test cells only on day 56, and day 63 post-differentiation initiation, results for these assays in the presence or absence of 5 ng/ml IL-15 is depicted in FIGS. 30C and 30D respectively. These results support the conclusion of mbIL-15 KI iNKs persisting and facilitating tumor cell killing in the absence or presence of exogenous IL-15.

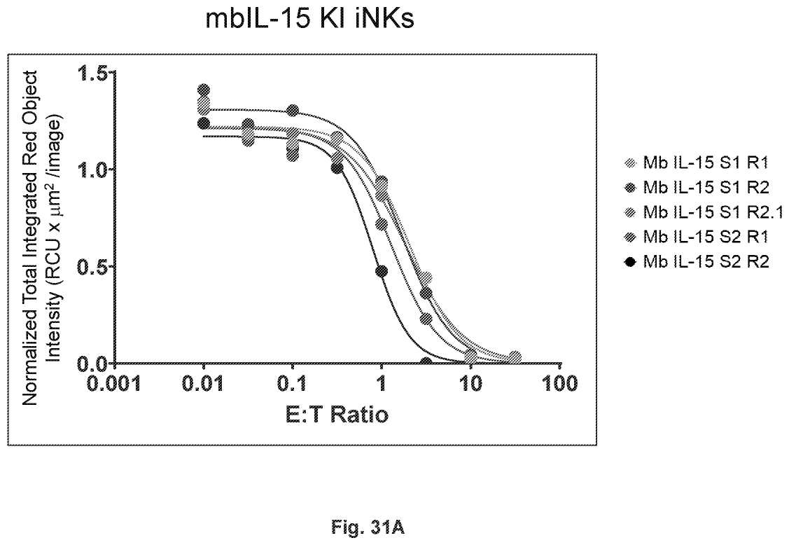

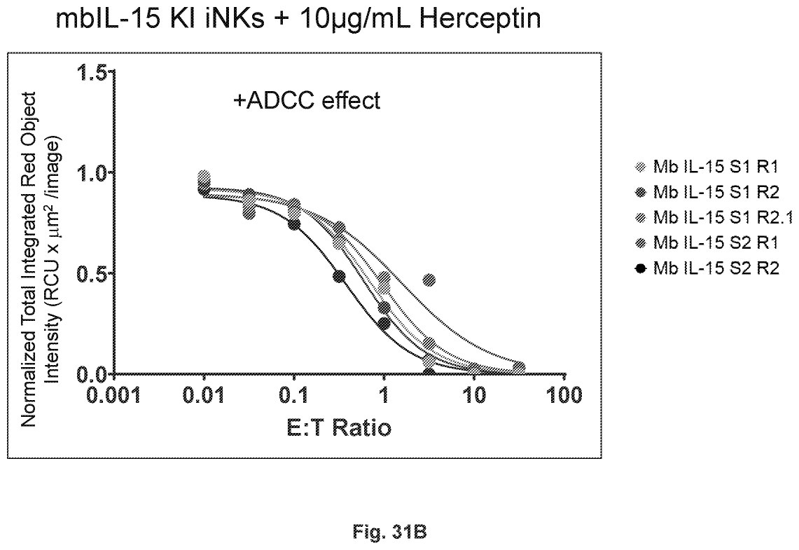

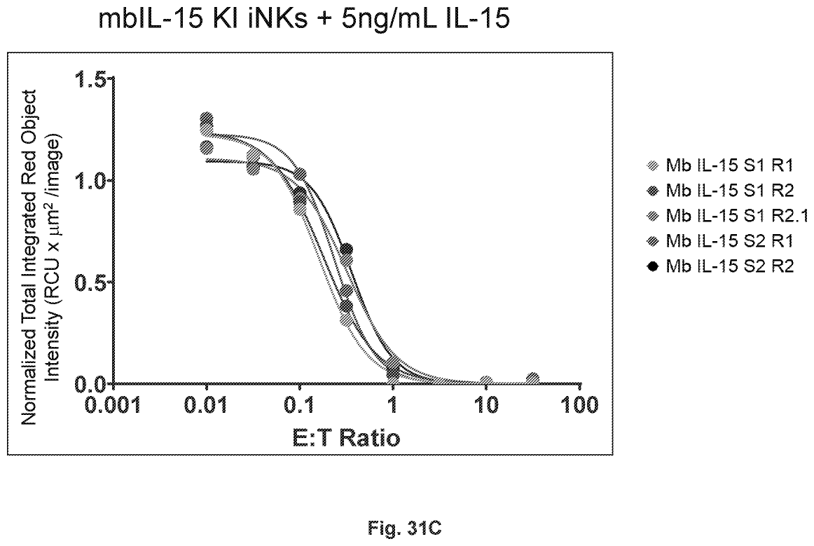

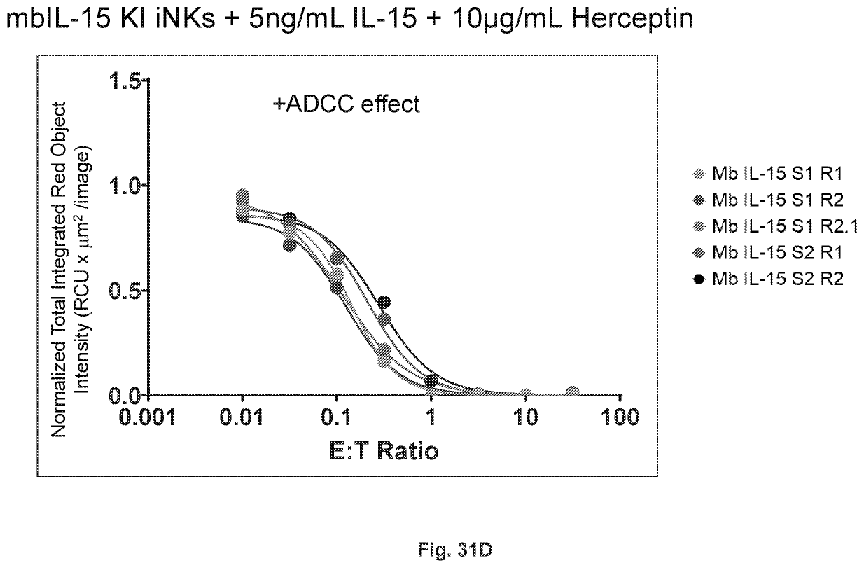

In addition, mbIL-15 KI iNK cells at later stages of differentiation (day 63 post-differentiation initiation for Set 1 (S1) and day 56 post-differentiation initiation for Set 2 (S2)) were also challenged in 3D spheroid killing assays as described above. Cells were tested in the presence or absence of 10 μg/ml Herceptin and/or 5 ng/mL exogenous IL-15. As shown in Table 19 and FIG. 31A-31D, mbIL-15 KI iNK cells exhibited high tumor cell killing efficiency, particularly when coupled with antibody therapy. At day 63, all mbIL-15 KI iNK cells did not express detectable levels of IL-15Ra; at Day 56, only one mbIL-15 KI iNK cell line (Mb IL-15 S2 R2) expressed detectable levels of IL-15Ra (data not shown).

The cumulative results of certain 3D spheroid killing assays for mbIL-15 KI iNKs and control WT iNK cells is depicted in FIG. 32. Two independent bulk edited populations of iPSCs (Set 1 (S1) and Set 2 (S2)) comprising mbIL-15 knock-in at the GAPDH gene were differentiated into iNK cells (day 39 and 49 of iPSC differentiation for Set 1, and day 42 of iPSC differentiation for Set 2) These iNK cells significantly reduced tumor cell spheroid size when compared to differentiated WT parental cell iNKs in the absence of exogenous IL-15 (P=0.034, +/− standard deviation, unpaired t-test). The differentiated knock-in mbIL-15 iNK cells also trended towards significant reduction of tumor cell spheroid size when compared to differentiated WT parental cells in the presence of 5 ng/ml exogenous IL-15 (P=0.052, +/− standard deviation, unpaired t-test). These results show that populations of iNK cells comprising mbIL-15 knock-in at the GAPDH locus using the methods described herein perform better in killing tumor cells in the absence of exogenously added IL-15 compared to populations of unedited iNK cells.

TABLE 18
mbIL-15 KI iNK 3D spheroid killing with IL-15

| | EC50 with | EC50 with |
|---|---|---|
| Cell Line | 0 ng/mL IL-15 | 5 ng/mL IL-15 |
| Mb IL-15 S1 | 9.575 | 1.648 |
| Mb-IL-15 S2 | 11.05 | 1.646 |
| WT iNK (PCS) 1 | 20.71 | 4.378 |
| WT iNK (PCS) 2 | 20.99 | 3.213 |

TABLE 19
mbIL-15 KI iNK 3D spheroid killing with Herceptin and/or IL-15

| | | | EC50 with | EC50 with |
|---|---|---|---|---|
| | | | 5 ng/ml | 5 ng/ml |
| | EC50 with | EC50 with | IL-15 and | IL-15 and |
| | 0 μg/mL | 10 μg/mL | 0 μg/mL | 10 μg/mL |
| Cell Line | Herceptin | Herceptin | Herceptin | Herceptin |
| Mb IL-15 Set1 Rep1 | 2.055 | 0.6936 | 0.16515 | 0.1423 |
| Mb IL-15 Set1 Rep2 | 1.701 | 0.5903 | 0.1794 | 0.1247 |
| Mb IL-15 Set1 Rep2.1 | 1.848 | 0.9570 | 0.3187 | 0.1153 |
| Mb IL-15 Set2 Rep1 | 1.291 | 1.589 | 0.2339 | 0.2096 |
| Mb IL-15 Set2 Rep2 | 0.8026 | 0.3783 | 0.3605 | 0.2778 |

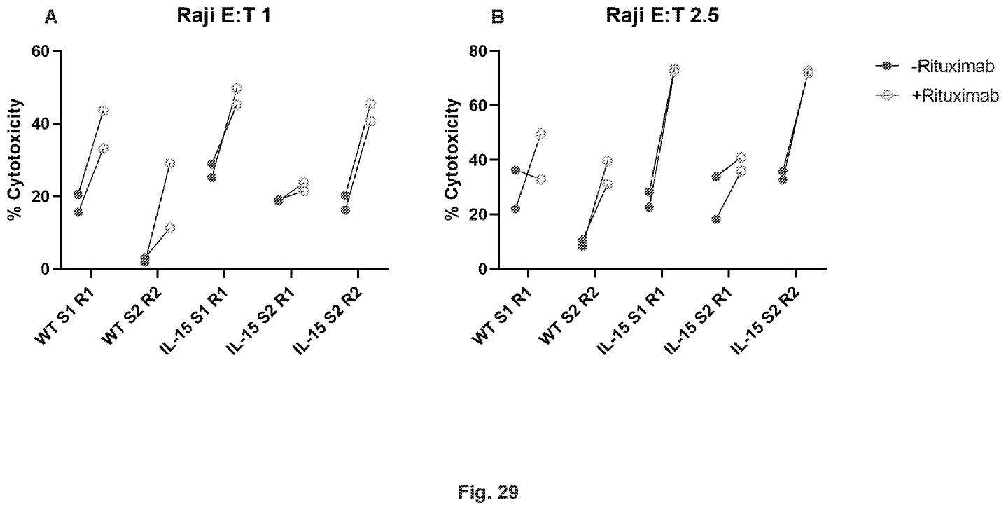

In addition, the mbIL-15 KI iNK cells at later stages of differentiation (day 63 post-differentiation initiation for Set 1 (S1) and day 56 post-differentiation initiation for Set 2 (S2)) were also challenged with hematological cancer cells (e.g., Raji cells). Two biological replicate populations of mbIL-15 KI NK cells (S1 and S2) were tested in the presence or absence of 10 μg/ml rituximab. As shown in FIG. 29, mbIL-15 KI iNK cells exhibited high tumor cell killing efficiency, particularly when coupled with antibody therapy. This killing capacity of these cells is significant, as Raji cells are naturally resistant to NK cells, but the mbIL-15 KI iNK cells in combination with antibody were able to find and kill these cells.

### Example 14: Knock-In of Multicistronic CD16, IL-15, and/or IL-15Rα Sequences at a Suitable Essential Gene Loci

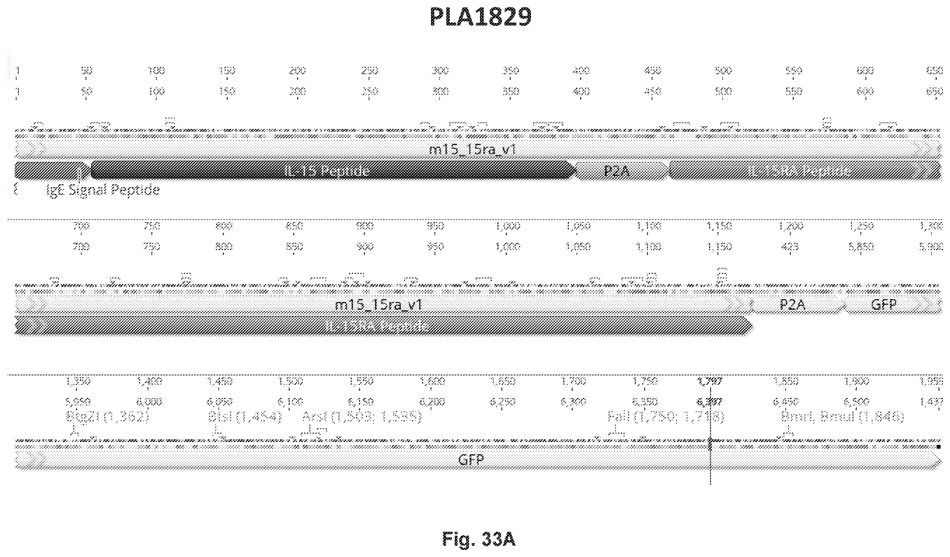

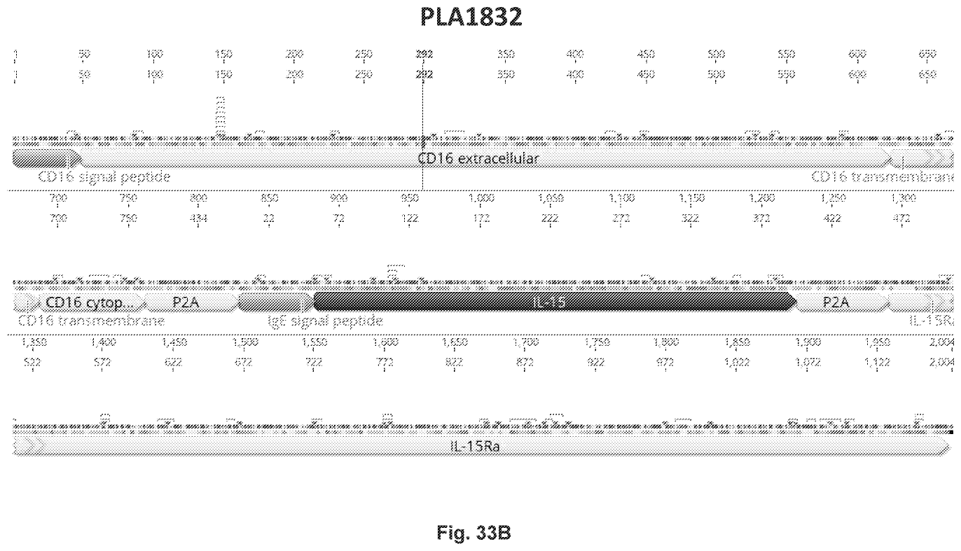

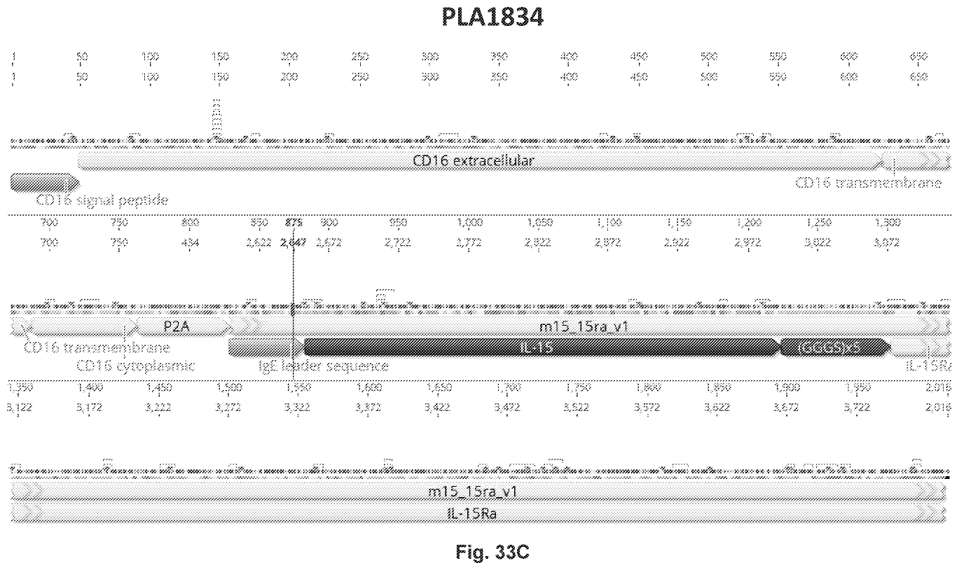

As described above in Example 2, genes of interest (GOI) may be integrated as a cargo sequence into suitable essential gene loci using methods described herein. In certain embodiments, multiple GOIs may be combined into a bicistronic or multicistronic knock-in cargo sequence. FIG. 33A depicts a portion of PLA1829 (comprising donor template SEQ ID NO: 208) comprising a bicistronic knock-in cargo sequence that was utilized for targeted integration at the GAPDH gene comprising an IL-15 peptide sequence, an IL-15Rα peptide sequence, and a GFP peptide sequence (SEQ ID NOs: 187, 189, and 195 respectively). Each of these peptide sequences were separate by a P2A sequence. Depicted in FIG. 33B is a portion of PLA1832 (comprising donor template SEQ ID NO: 209) comprising a multicistronic knock-in cargo sequence that was utilized for targeted integration at the GAPDH gene comprising a CD16 peptide sequence, an IL-15 peptide sequence, and an IL-15Rα peptide sequence (SEQ ID NOs:184, 187, and 189 respectively). Each of these peptide sequences were separate by a P2A sequence. Depicted in FIG. 33C is a portion of PLA1834 (comprising donor template SEQ ID NO: 212) comprising a bicistronic knock-in cargo sequence that was utilized for targeted integration at the GAPDH gene comprising a CD16 peptide sequence, and an mbIL-15 peptide sequence (an IL-15 sequence fused to an IL-15Rα sequence as depicted in FIG. 26) (SEQ ID NOs: 184 and 190 respectively) separated by a P2A sequence.

The knock-in cargo sequences described in FIG. 33A-33C are comprised within Plasmids 1829, 1832, and 1834 respectively (comprising donor template SEQ ID NOs: 208, 209, and 212). PSCs were edited using the exemplary system illustrated in FIGS. 3A, 3B, and 3C, and described in Example 2. In brief, the GAPDH gene was targeted in iPSCs using AsCpf1 (AsCas12a (SEQ ID NO: 62)) and a guide RNA (RSQ22337 (SEQ ID NO: 95)), resulting in a double-strand break towards the 5′ end of the last exon of GAPDH (exon 9). The CRISPR/Cas nuclease and guide RNA were introduced by nucleofection (electroporation) of a ribonucleoprotein (RNP) according to known methods. The cells were also contacted with a double stranded DNA donor template (dsDNA plasmid (PLA1829, PLA1832, or PLA1834 respectively)) that included a donor template (SEQ ID NO: s: 208, 209, and 212) comprising in 5′-to-3′ order, a 5′ homology arm approximately 500 bp in length (comprising a 3′ portion of exon 8, intron 8, and a 5′ codon-optimized coding portion of exon 9 optimized to prevent further binding of the gRNA targeting domain sequence of the guide RNA (RSQ22337)), an in-frame sequence encoding the P2A self-cleaving peptide (“P2A”), an in-frame coding sequence as described above (“Cargo”), a stop codon and polyA signal sequence, and a 3′ homology arm approximately 500 bp in length (comprising a coding portion of exon 9 including a stop codon, the 3′ non-coding exonic region of exon 9, and a portion of the downstream intergenic sequence) (as shown in FIG. 3B). Four unique nucleofection events were conducted (corresponding to RNP and PLA1829, RNP and PLA1832, RNP and PLA1834, and RNP with no plasmid control) and cells were plated at clonal density. Colonies were propagated for analysis of TI using ddPCR.

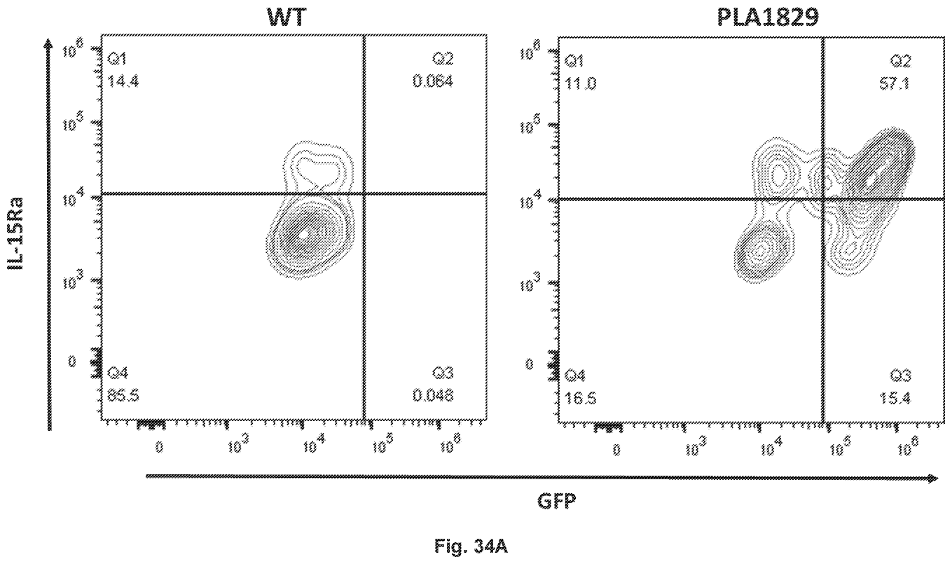

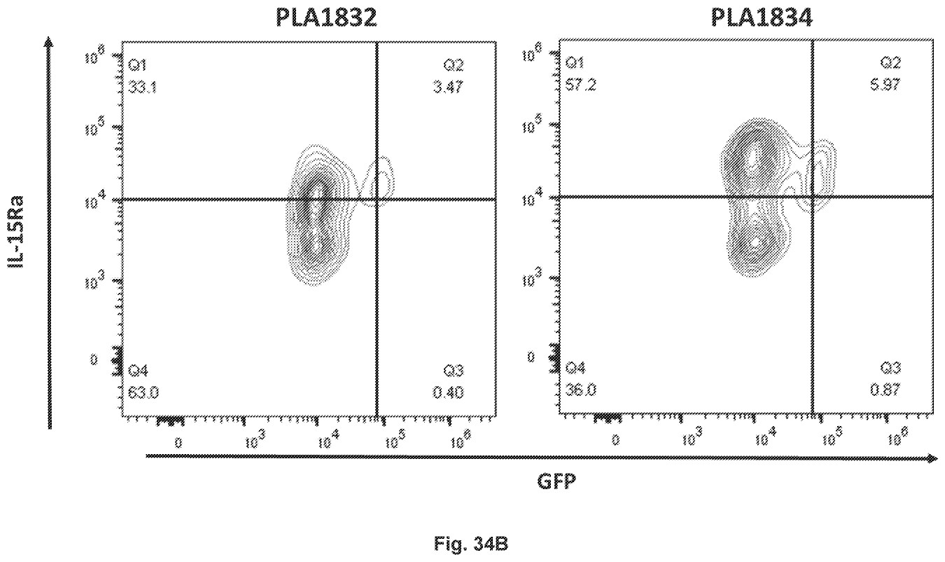

Following TI, transformed iPSCs (edited clones) with KI of PLA1829, PLA1832 or PLA1834 cargo sequences, or control WT parental cells transformed with RNP alone, were analyzed using flow cytometry seven days after transformation (see FIGS. 34A and 34B). The levels of GFP and IL-15Rα expression were measured in bulk edited iPSC populations. As shown in FIG. 34A, approximately 57% of cells transformed with PLA1829 expressed both IL-15Rα and GFP, while control cells had no GFP expression and approximately 14.4% IL-15Rα expression levels. As shown in FIG. 34B, approximately 33.1% of cells transformed with PLA1832, and approximately 57.2% of cells transformed with PLA1834 expressed IL-15Rα; neither of these cell populations displayed appreciable GFP levels, as expected as the respective donor templates did not comprise GFP. The expression of these cargo proteins can be used as a proxy for determining successful transformation, editing, and/or integration.

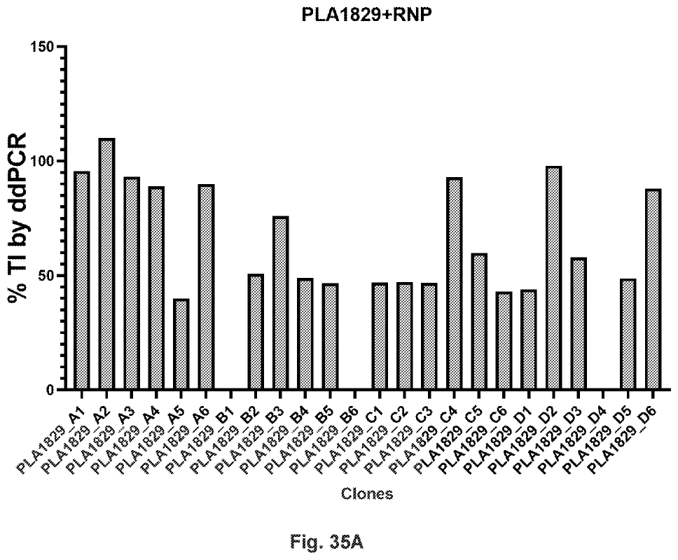

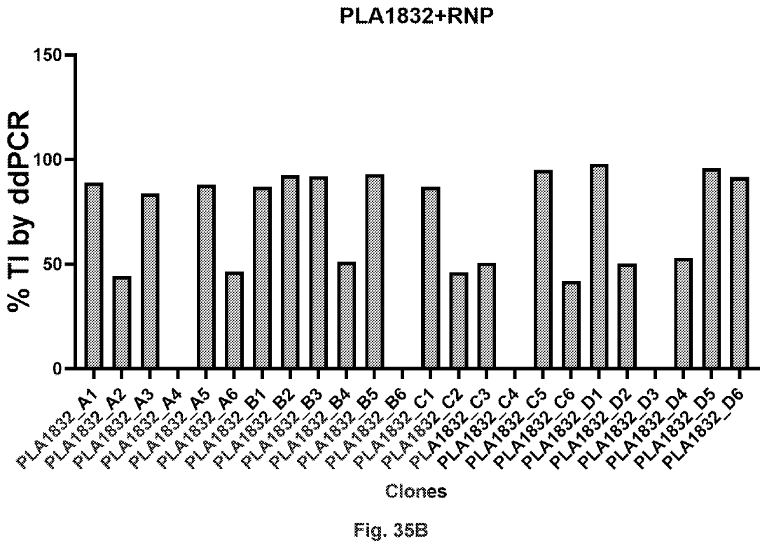

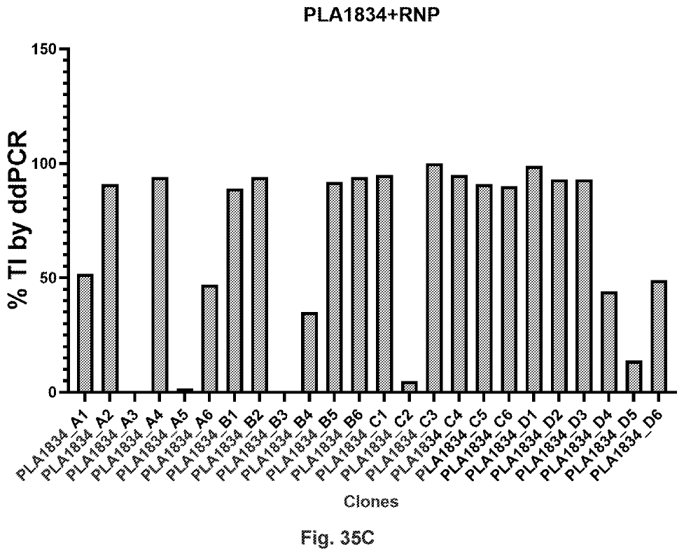

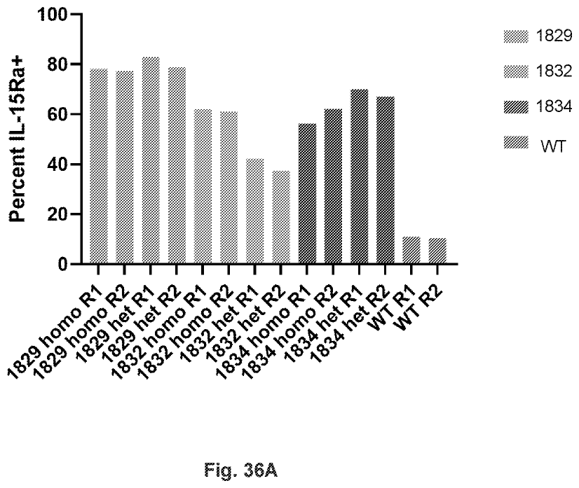

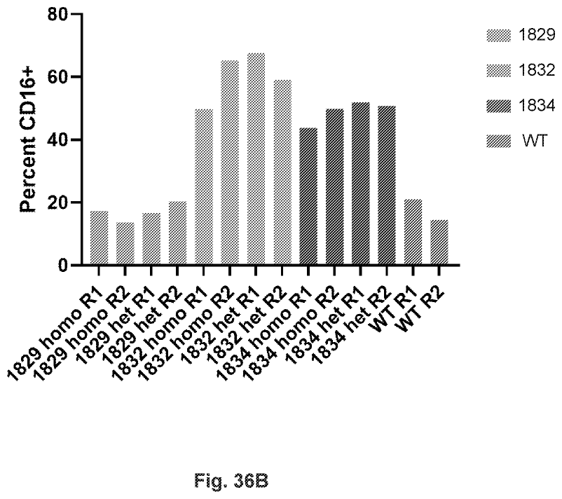

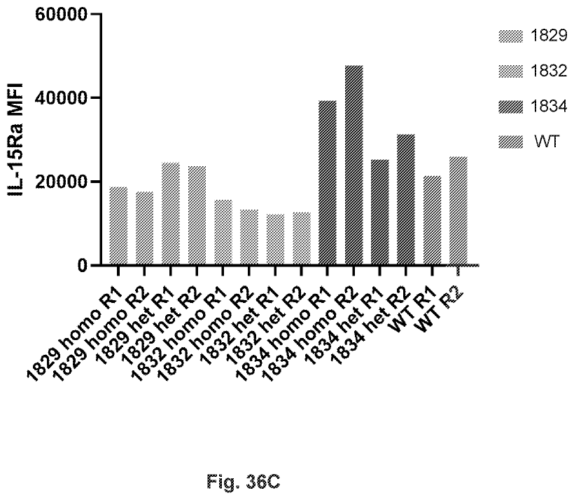

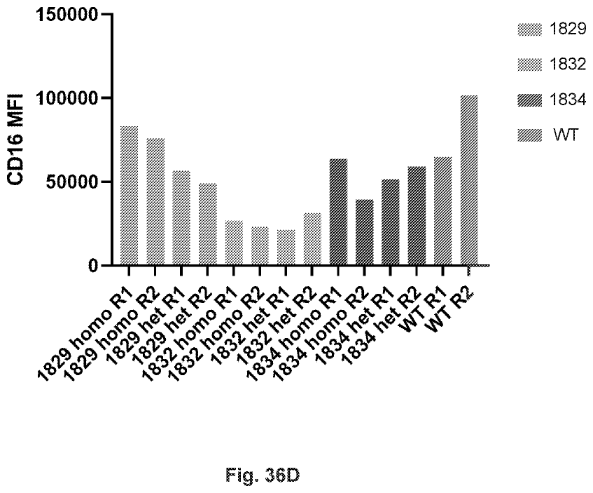

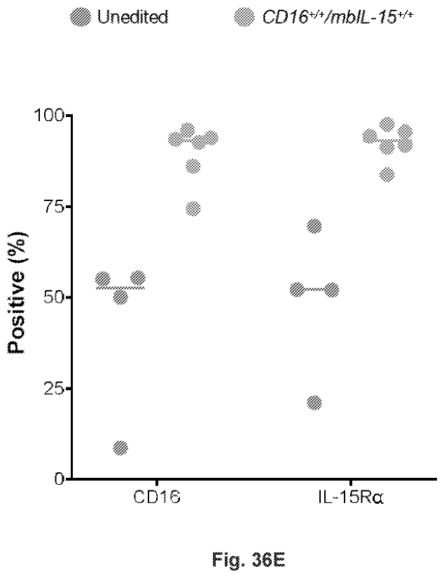

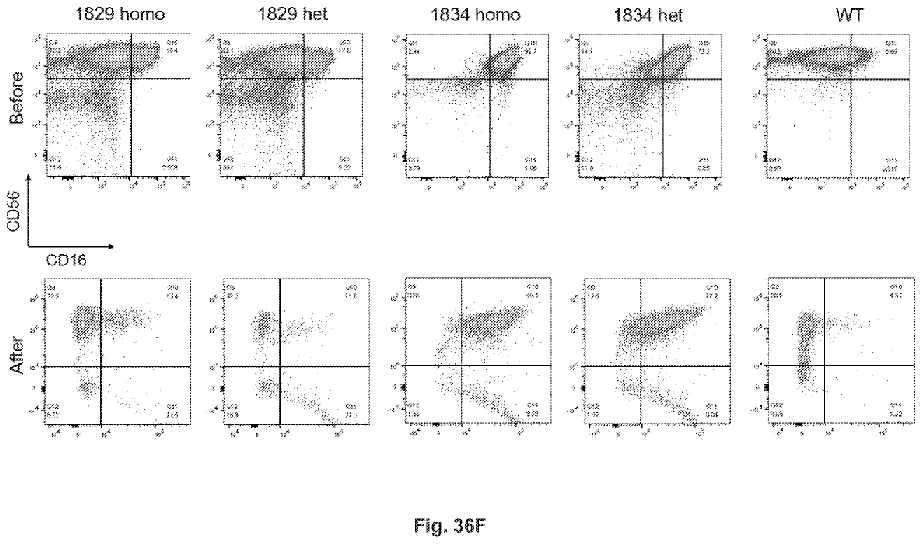

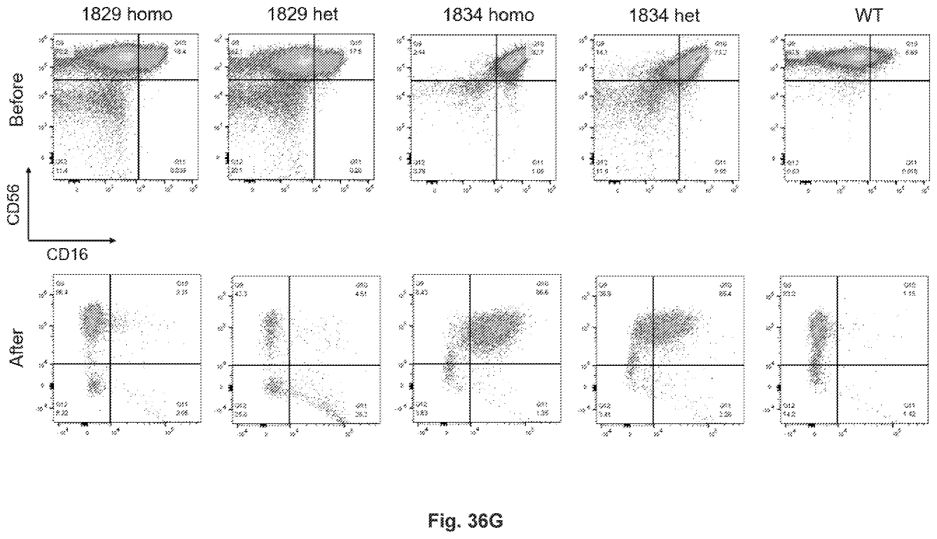

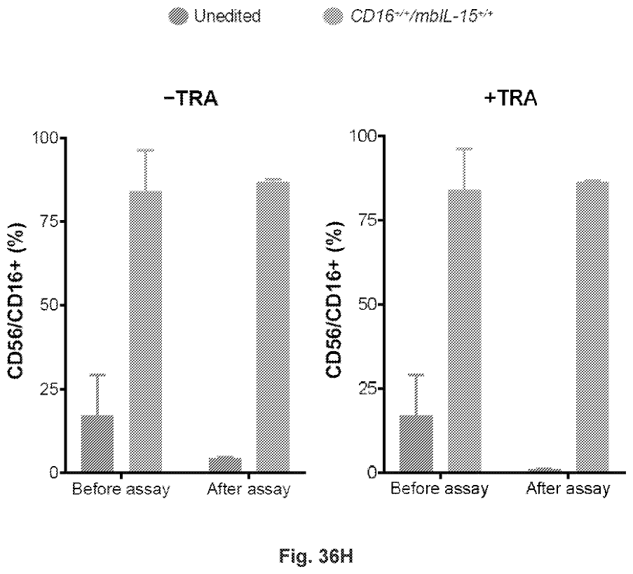

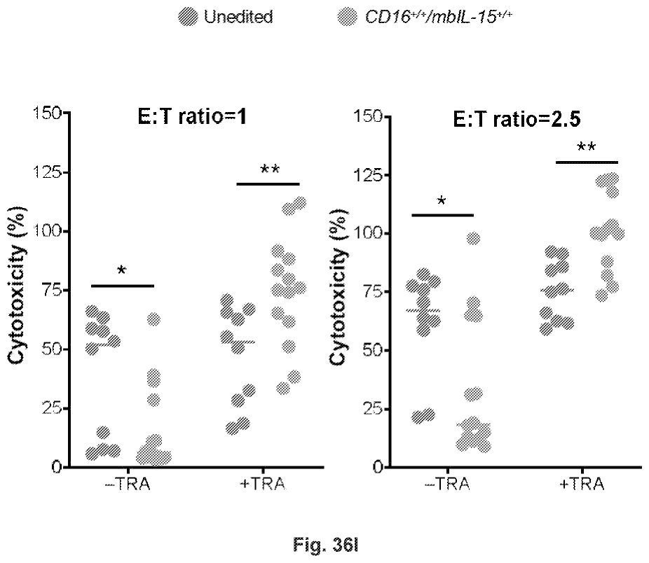

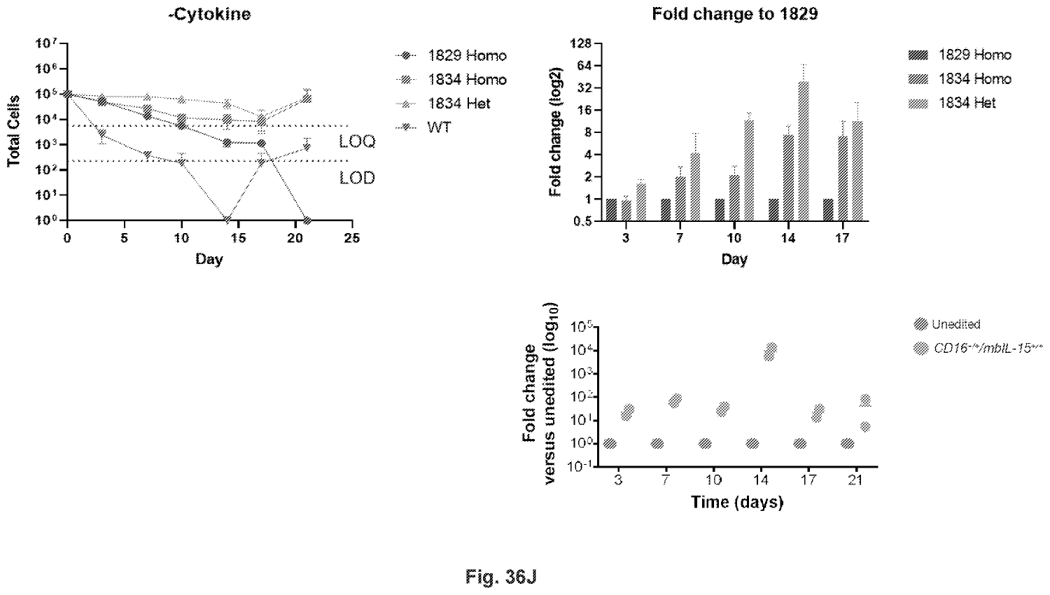

FIG. 35A-35C depicts the genotypes for 24 of the colonies transformed with PLA1829, PLA1832, or PLA1834 (comprising donor template SEQ ID NOs: 208, 209, and 212) respectively and compared to wild-type cells. Measured with ddPCR, cells with ˜85-100% TI are categorized as homozygous, 40-60% are categorized as heterozygous, while those with very low or no signal are categorized as wild type. The colonies were propagated after transformation, and cell populations were then differentiated to iNK cells using a spin embryoid method as known in the art. Shown in FIG. 36A-36D are exemplary flow cytometry results measuring the percentage of cells expressing IL-15Rα and/or CD16, and the median fluorescence intensity (MFI) of IL-15Rα and/or CD16 at day 32 of the iNK differentiation process. As shown in FIG. 36A, transformation with PLA1829, PLA1832, or PLA1834 enabled surface expression of IL-15Rα in heterozygous or homozygous colonies at significantly higher proportions than iNKs differentiated from control WT parental cells. As shown in FIG. 36B, transformation with PLA1832 or PLA1834 enabled surface expression of CD16 in heterozygous or homozygous colonies at significantly higher proportions than iNKs differentiated from control WT parental cells, as cells transformed with the PLA1829 cargo sequence do not comprise a CD16 cargo sequence. As shown in FIG. 36C, transformation with PLA1834 enabled higher MFI of IL-15Rα in heterozygous or homozygous colonies when compared to iNKs differentiated from control WT parental cells, or cells transformed with PLA1829 or PLA1832. As shown in FIG. 36D, transformation with PLA1832 or PLA1834 enabled surface expression of CD16 in heterozygous or homozygous colonies. These data show that the methods described herein can be used to knock-in a multicistronic cargo containing numerous genes of interest into an essential gene such as GAPDH, leading to expression of the genes of interest in the edited cells. These data also clearly demonstrate the constitutive nature of cargo expression from the GAPDH locus.

The differentiated iNK cells were also used in lactate dehydrogenase (LDH) killing assays, and iNK cells were assessed for surface expression of CD16 by flow cytometry, before and after the cytotoxicity assay (E:T ratio of 1 or 2.5). As shown in FIG. 36F (E:T ratio of 2.5), in the absence of trastuzumab (Herceptin), WT cells and cells transformed with PLA1829 (without CD16 KI) showed small decreases in the surface level expression of CD16 after coming into contact with the SK-OV-3 cells in the LDH assay while cells transformed with PLA1834 (and thus having CD16 KI) showed minimal reduction. In the presence of trastuzumab, cells transformed with PLA1834 demonstrated a similar minimal reduction in the level of CD16 after coming into contact with the SK-OV-3 cells in the LDH assay; however, a marked difference in CD16 surface expression was observed for WT cells and cells transformed with PLA1829 (without CD16 KI) after coming into contact with the SK-OV-3 cells in the LDH assay (FIG. 36G, E:T ratio of 2.5). Further experimental replicates confirmed that homozygous colonies of cells transformed with PLA1834 largely maintained CD16 surface expression after contact with SK-OV-3 cells in the LDH assay, in the absence or presence of trastuzumab, whereas unedited (WT) parental cells displayed substantial decreases in CD16 surface expression after contact with SK-OV-3 cells (FIG. 36H). Overall, the results show that KI of a cleavable CD16 construct at GAPDH can lead to high levels of CD16 surface expression in KI iNK cells, and there is minimal CD16 shedding from the CD16 KI iNK cells after they contact tumor cells. Additionally, as shown in FIG. 36I, homozygous PLA1834-transformed iNK cells exhibited greater cytotoxicity than unedited (WT) iNK cells in the presence and absence of trastuzumab at E:T ratios of 1 and 2.5 in the LDH assay.

In addition, differentiated iNK cells (unedited (WT) cells) and homozygous colonies of PLA1834-transformed (CD16^{+/+}/mbIL-15^{+/+}) cells were used in 3D tumor spheroid killing assays as described in Example 11 and schematized in FIG. 20. Cells were tested for 100 hours at an E:T ratio of 10 and in the absence or presence of 10 μg/ml trastuzumab. CD16^{+/+}/mbIL-15^{+/+}iNK cells elicited greater reduction in tumor spheroid size than unedited iNK cells without or with trastuzumab (FIG. 37B). As shown in FIG. 37C, CD16^{+/+}/mbIL-15^{+/+} iNK cells showed enhanced cytotoxicity in the 3D tumor spheroid assay compared with unedited iNK cells or peripheral blood NK cells across a range of E:T ratios in the presence of 10 μg/ml trastuzumab and 5 ng/ml exogenous IL-15. The average IC50 (as measured via E:T ratio) of the CD16^{+/+}/mbIL-15^{+/+}iNK cells was significantly lower than the unedited iNK cells in the absence or presence of trastuzumab (FIG. 37C). These 3D tumor spheroid killing assay results further confirm that the CD16^{+/+}/mbIL-15^{+/+} (homozygous PLA1834-transformed) iNK cells demonstrate greater cytotoxicity of tumor cells and are more efficient at tumor cell killing than unedited (WT) iNK cells in the presence or absence of trastuzumab or in the presence of the combination of trastuzumab and exogenous IL-15.

Membrane bound IL-15 also mediated iNK cell survival for a prolonged period of time without the support of homeostatic cytokines. Starting at Day 43 post-differentiation, iNK cells were maintained in the absence of IL-2 or IL-15 for three weeks. As shown in FIG. 36J, in contrast to WT cells, the total number of iNK cells transformed with PLA1834 (heterozygote and homozygote KI cells) remained stable over the three-week assay. These data show that cells transformed with PLA1834 demonstrated superior persistence in the absence of cytokines compared to WT cells and to cells transformed with PLA1829.

### Example 15: In Vivo Assay of Bicistronic CD16 and mbIL-15 Sequences at a Suitable Essential Gene Loci

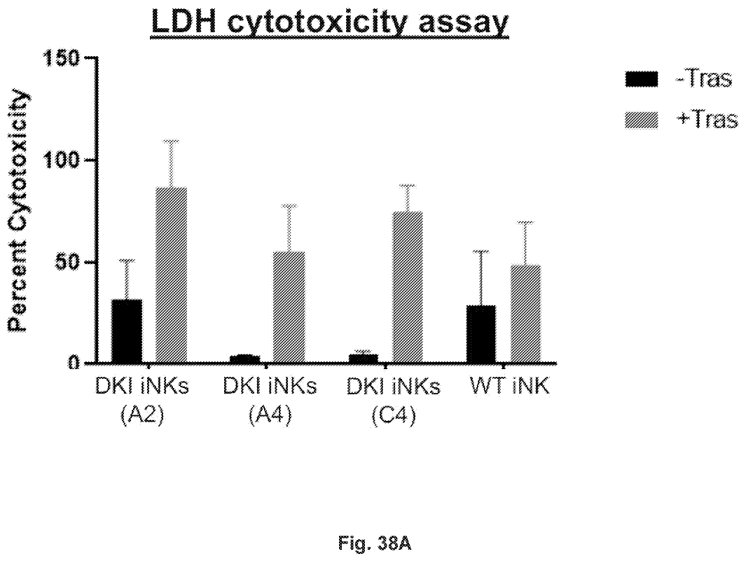

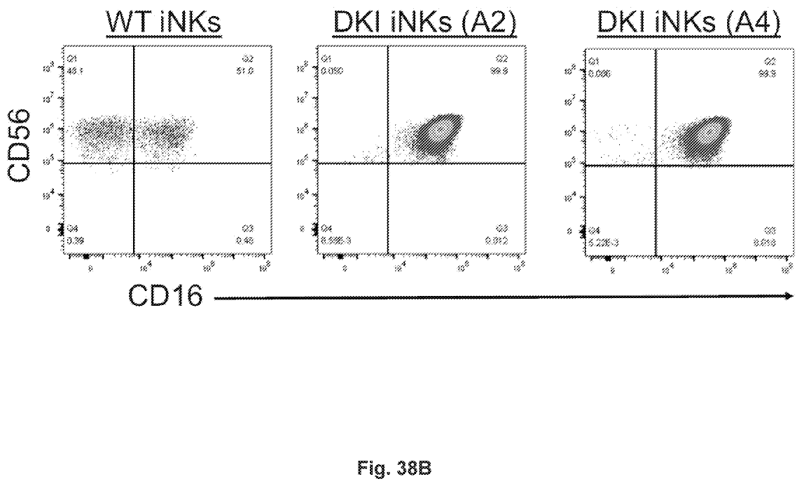

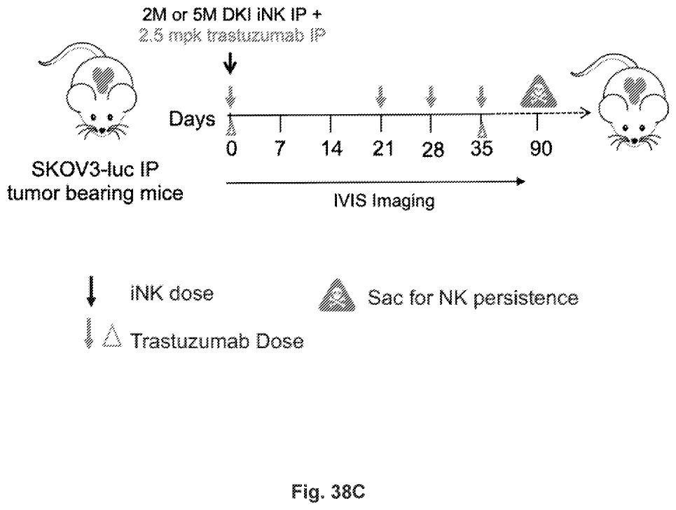

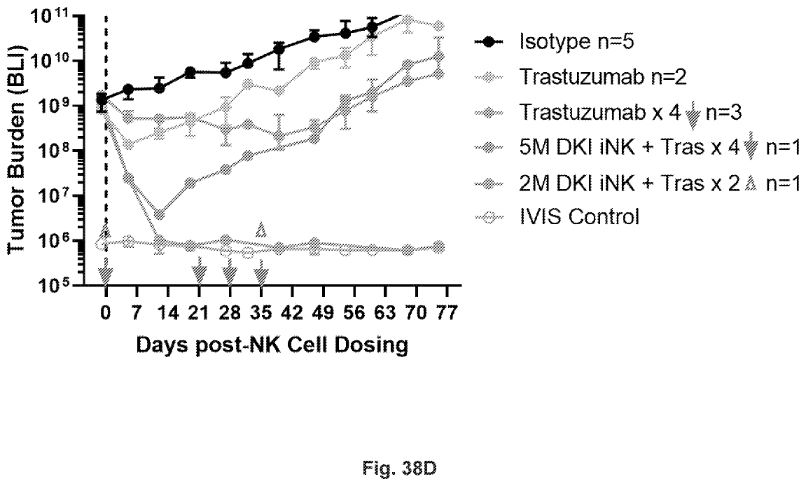

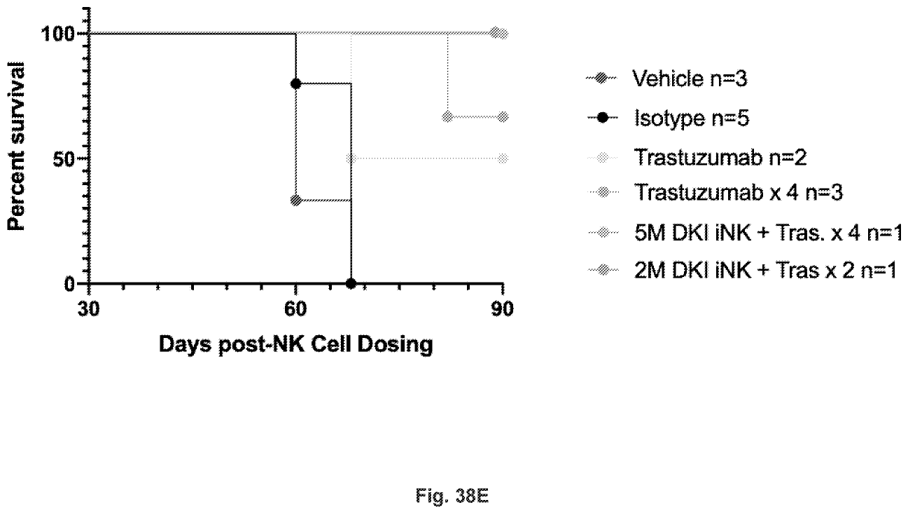

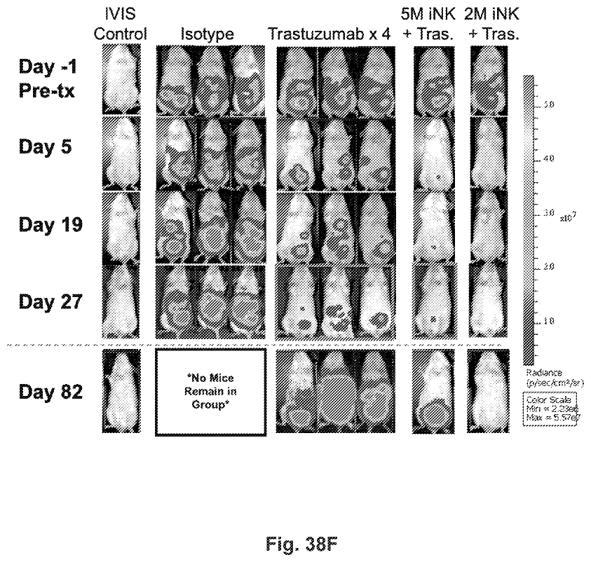

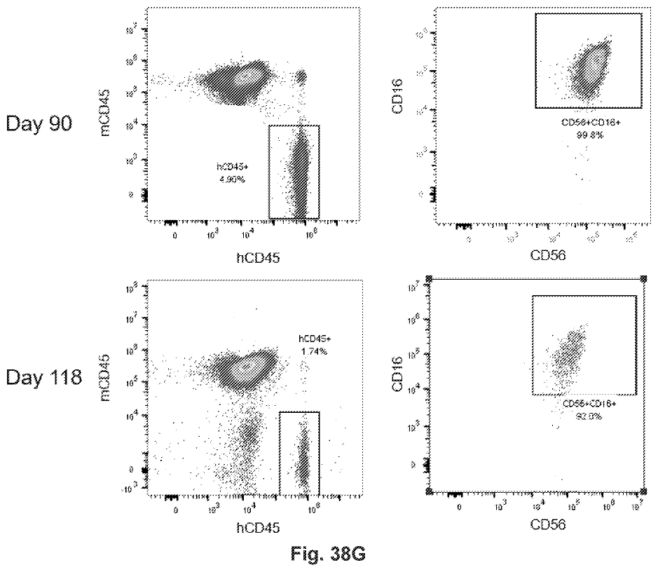

Plasmid PLA1834 was used to generate iPSC-derived NK (iNK) cells comprising mbIL-15/CD16 double knock-in (DKI), as described in Example 14. From these mbIL-15/CD16 DKI iNK cells, three homozygous (CD16^{+/+}/mbIL-15^{+/+}) clones (A2, A4, C4) were selected for testing in an in vitro lactate dehydrogenase (LDH) release assay to assess cell cytotoxicity against SK-OV-3 tumor cells as described in Example 14. An unedited (WT) iNK cell control was also tested. Cells were tested in the presence and absence of 10 μg/ml trastuzumab and at an E:T ratio of 1. As shown in FIG. 38A, mbIL-15/CD16 (CD16^{+/+}/mbIL-15^{+/+}) DKI iNK cells demonstrated significant increases in average percent cytotoxicity in the presence of trastuzumab as compared to average percentage cytotoxicity seen in the absence trastuzumab, confirming the potent in vitro tumor killing activity of these cells described in Example 14. The mbIL-15/CD16 (CD16^{+/+}/mbIL-15^{+/+}) DKI iNK cells were also examined by flow cytometry for expression of CD16. Samples of unedited (WT) or DKI iNK cells (clones A2 and A4) were pre-gated for alive hCD45+ cells and then examined for CD56 and CD16 expression. As shown in FIG. 38B, WT and DKI iNK cells were highly pure CD56+ NK cells. Moreover, both clones of the mbIL-15/CD16 (CD16^{+/+}/mbIL-15^{+/+}) DKI iNK cells had approximately 100% of cells expressing high levels of CD16, while approximately half of the WT iNK cells expressed CD16.

Following confirmation of cytotoxicity in the LDH assay and of high CD16 expression, mbIL-15/CD16 (CD16^{+/+}/mbIL-15^{+/+}) DKI iNK cells were assayed for their ability to kill tumor targets in an in vivo mouse model. FIG. 38C depicts a schematic of the assay. Mice were inoculated with 0.25×10⁶ SK-OV-3 cells engineered to express luciferase (SKOV3-luc). Following 2-6 days to allow for establishment of the tumors, mice were imaged using an in vivo imaging system (IVIS) to establish pre-treatment (day −1) tumor burden and then randomized into treatment groups. After 1 additional day (on day 0), mice were injected intraperitoneally (IP) with 2×10⁶ (2M) or 5×10⁶ (5M) mbIL-15/CD16 (CD16^{+/+}/mbIL-15^{+/+}) DKI iNK cells+2.5 mpk trastuzumab, 2.5 mpk trastuzumab alone, an isotype control, or a vehicle control. As depicted in FIG. 38C, one treatment group (mice injected with 5M mbIL-15/CD16 (CD16^{+/+}/mbIL-15^{+/+}) DKI iNK cells) received an additional dose of trastuzumab on day 35, and another treatment group (mice injected with 2M mbIL-15/CD16 (CD16^{+/+}/mbIL-15^{+/+}) DKI iNK cells) received additional doses of trastuzumab on days 21, 28, and 35. Following day 0, the mice were imaged weekly using IVIS to assess tumor burden over time. Mice were followed for up to 90 days.

The average tumor burden over time as measured by bioluminescent imaging (BLI) via IVIS is depicted in FIG. 38D, and percent survival over time is depicted in FIG. 38E. Representative bioluminescent imaging of the mice at various time points is displayed in FIG. 38F. The mouse dosed with 2×10⁶ mbIL-15/CD16 (CD16^{+/+}/mbIL-15^{+/+}) DKI iNK cells in combination with trastuzumab (2M DKI iNK+Tras) exhibited complete tumor clearance (FIGS. 38D and 38F) and prolonged survival (FIG. 38E). By contrast, the mice treated with trastuzumab alone or with the isotype control exhibited higher tumor burden and decreased survival. These data demonstrate that mbIL-15/CD16 (CD16^{+/+}/mbIL-15^{+/+}) DKI iNK cells actively kill tumor cells in an in vivo model and that treatment with both the mbIL-15/CD16 (CD16^{+/+}/mbIL-15^{+/+}) DKI iNK cells and trastuzumab results in better outcomes (e.g., prolonged survival, significantly greater tumor clearance) than dosing with trastuzumab alone.

The mouse dosed with 5×10⁶ mbIL-15/CD16 (CD16^{+/+}/mbIL-15^{+/+}) DKI iNK cells in combination with trastuzumab (5M DKI iNK+Tras) displayed a significant decrease in tumor burden by day 14, followed by an increase in tumor burden (FIGS. 38D and 38F). After sacrificing this mouse at day 90, the rebounded tumor was found to be located subcutaneously and not in the peritoneal cavity as experimentally intended. Thus, the intraperitoneally injected mbIL-15/CD16 (CD16^{+/+}/mbIL-15^{+/+}) DKI iNK cells were likely unable to access the tumor to the same extent as mbIL-15/CD16 (CD16^{+/+}/mbIL-15^{+/+}) DKI iNK cells administered to mice in other treatment groups. The presence of mbIL-15/CD16 (CD16^{+/+}/mbIL-15^{+/+}) DKI iNK cells in the peritoneal cavity of the mouse dosed with 5×10⁶ DKI iNK cells+trastuzumab was confirmed by flow cytometric analysis of the peritoneal lavage (FIG. 38G, top row). Moreover, the mbIL-15/CD16 (CD16^{+/+}/mbIL-15^{+/+}) DKI iNK cells expressed high levels of CD56 and CD16, with 100% of the cells expressing high levels of CD16 at day 90 (FIG. 38G, top right panel). Thus, the presence of the rebounded tumor in this mouse was unlikely due to a loss of the mbIL-15/CD16 (CD16^{+/+}/mbIL-15^{+/+}) DKI iNK cells. The mouse dosed with 2×10⁶ mbIL-15/CD16 (CD16^{+/+}/mbIL-15^{+/+}) DKI iNK cells+trastuzumab was sacrificed at day 118, and presence of mbIL-15/CD16 (CD16^{+/+}/mbIL-15^{+/+}) DKI iNK cells in the peritoneal cavity of the mouse was confirmed by flow cytometric analysis of the peritoneal lavage (FIG. 38G, bottom row). These cells expressed high levels of CD56 and CD16, with 92% of the cells expressing high levels of CD16 at day 118 (FIG. 38G, bottom right panel).

FIG. 38G demonstrates that knock-in of mbIL-15 prolongs the in vivo persistence of the iNK cells as compared to short-lived healthy donor-derived WT NK cells (data not shown). Further, the mbIL-15/CD16 (CD16^{+/+}/mbIL-15^{+/+}) DKI iNK cells continue to express high levels of CD16 on the cell surface due to the knock-in of CD16 (as described in Example 14), indicating that they retain the ability for ADCC mediated tumor killing in the presence of a therapeutic antibody (such as trastuzumab).

These data demonstrate that mbIL-15/CD16 (CD16^{+/+}/mbIL-15^{+/+}) DKI iNK cells are capable of persisting in vivo and maintaining high CD16 expression up to at least 118 days. This is notable, given that unedited NK cells have been reported to have limited persistence (see, e.g., Romee et al., Sci. Trans. Med. 8:357ra123357ra123 (2016)).

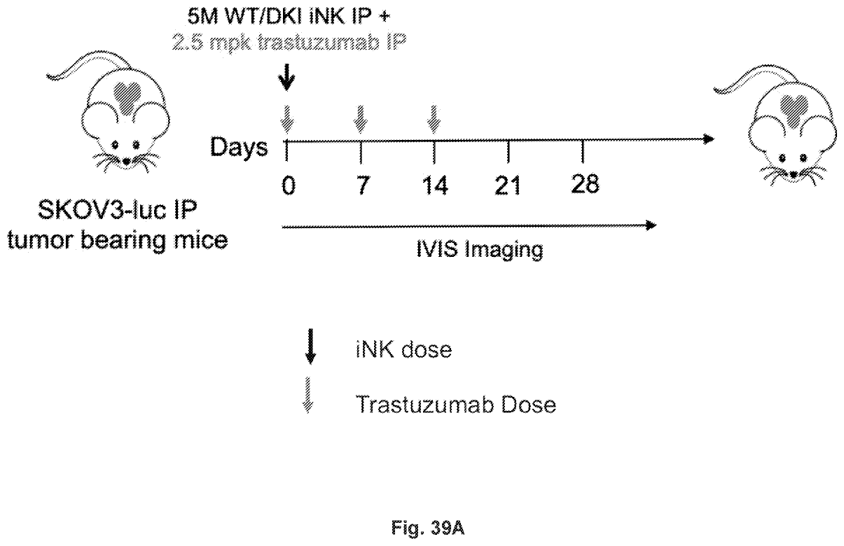

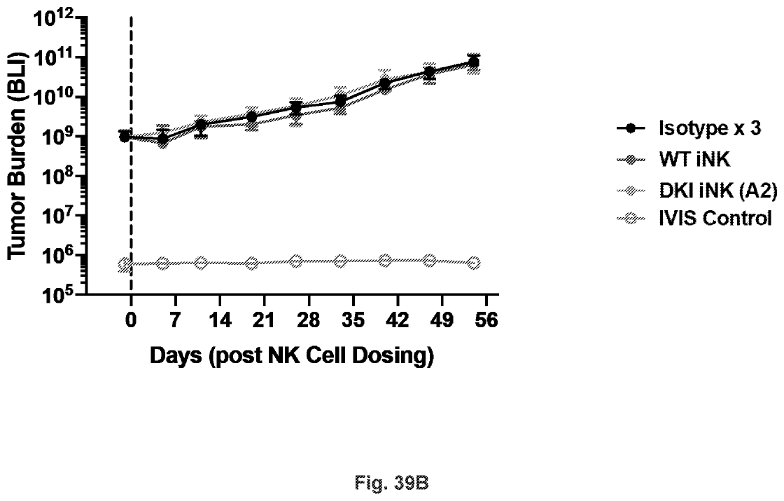

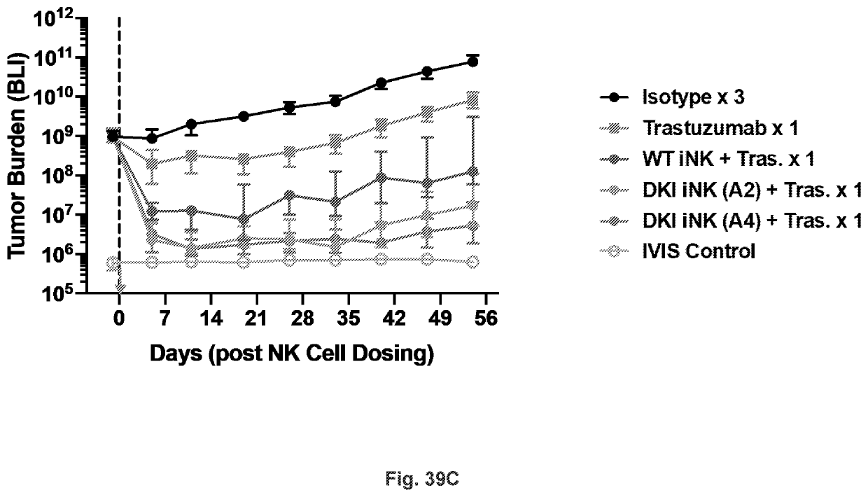

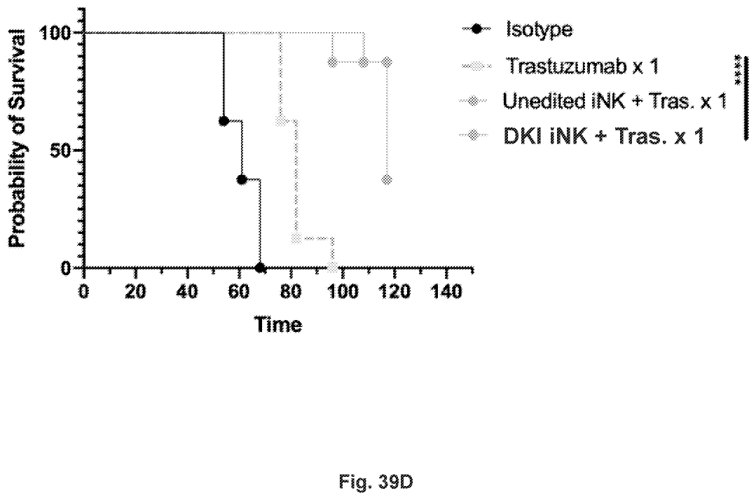

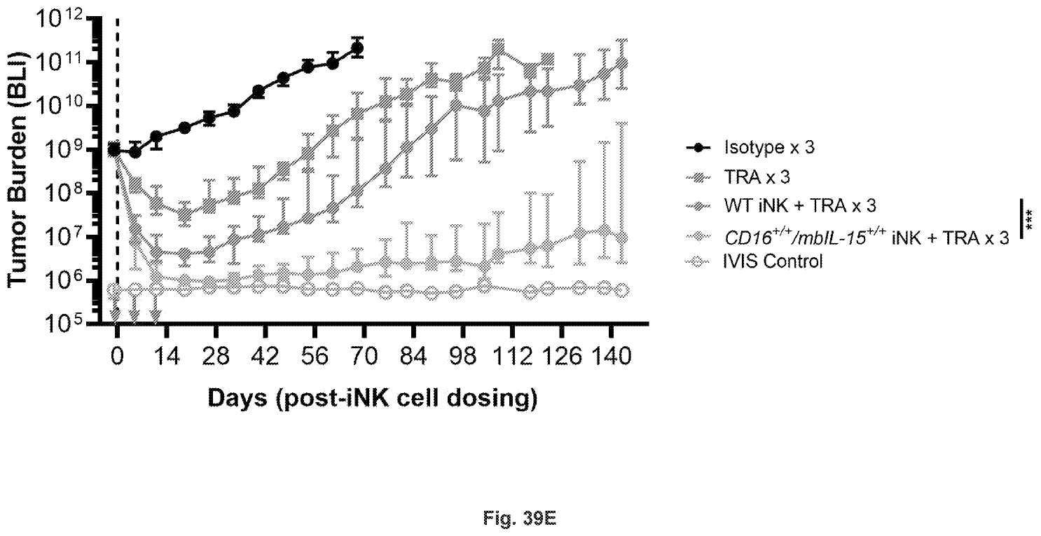

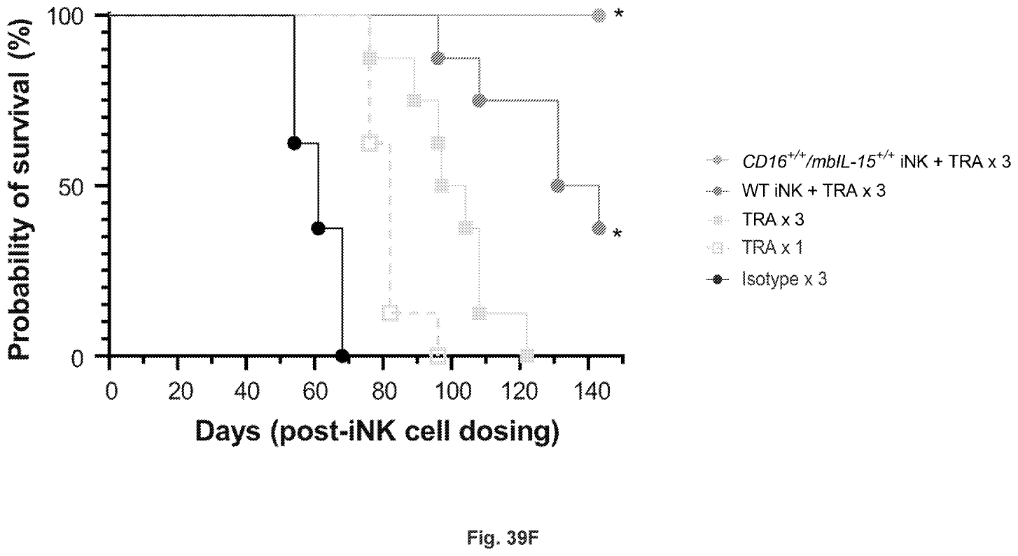

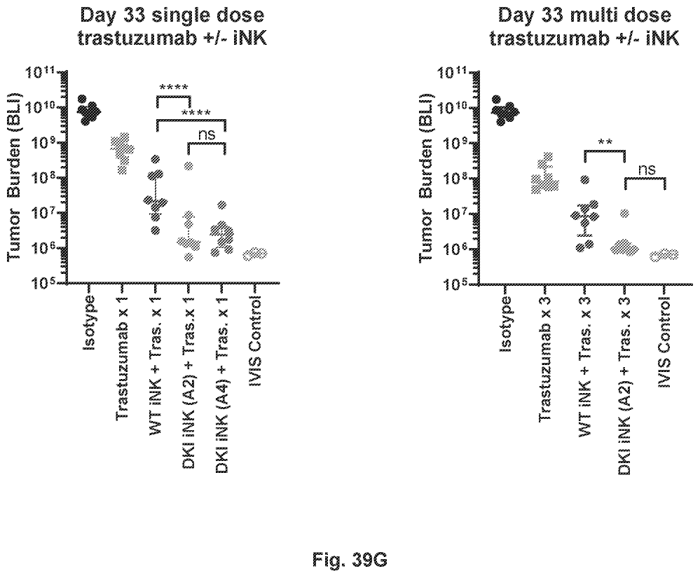

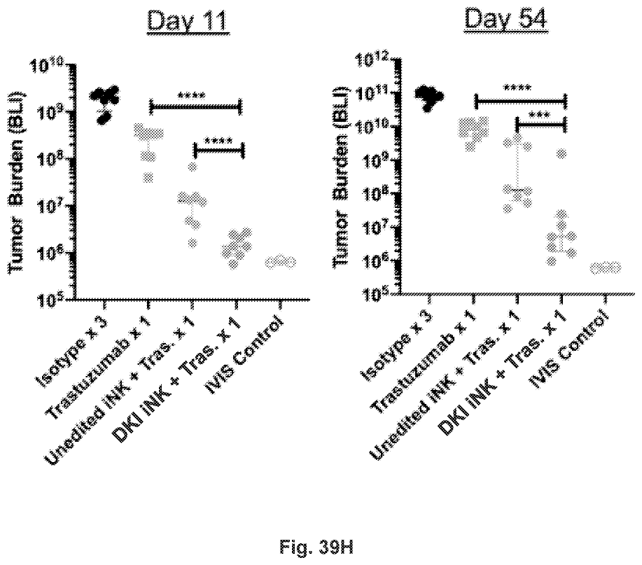

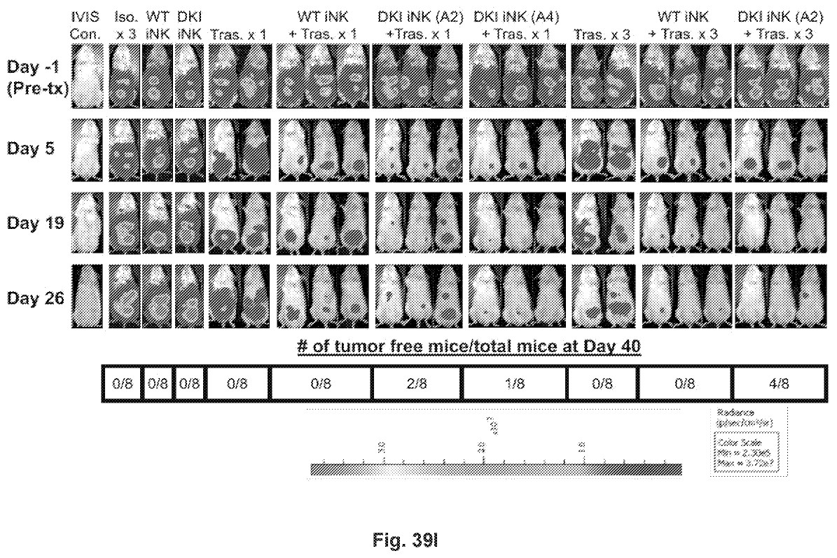

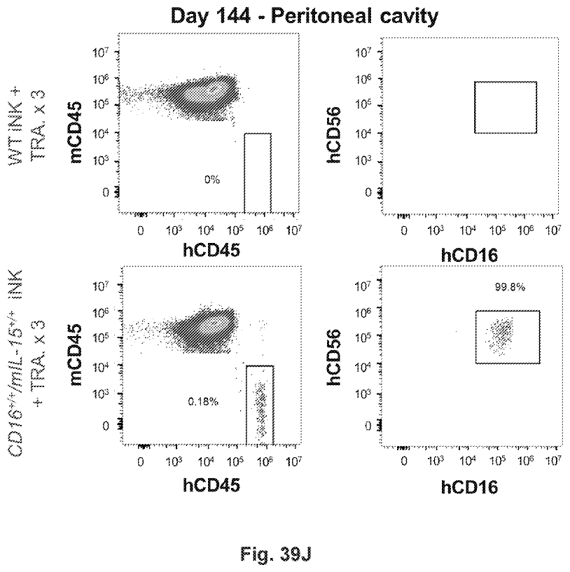

Further testing of the mbIL-15/CD16 (CD16^{+/+}/mbIL-15^{+/+}) DKI iNK cells in an in vivo mouse model was conducted as depicted in FIG. 39A. Mice were inoculated with 0.25×10⁶ SKOV3-luc cells. Following 2-6 davs to allow for establishment of the tumors, mice were imaged using an IVIS to establish pre-treatment (day −1) tumor burden and then randomized into treatment groups. After 1 additional day (on day 0), mice were injected intraperitoneally (IP) with 5×10⁶ unedited (WT) iNK cells, 5×10⁶ mbIL-15/CD16 (CD16^{+/+}/mbIL-15^{+/+}) DKI iNK cells (from clone A2 or A4), or no iNK cells for trastuzumab-alone or isotype control. One treatment group (“+Tras.×1”, “TRA×1”) received an IP injection of 2.5 mpk trastuzumab on day 0. Another treatment group (“+Tras.×3”, “+TRA×3”) received IP injections of 2.5 mpk trastuzumab on days 0, 7, and 14. Following day 0, the mice were imaged weekly using an IVIS to assess tumor burden over time.

The tumor burden over time as measured by bioluminescent imaging (BLI) via IVIS is depicted in FIGS. 39B, 39C, and 39E. Representative bioluminescent imaging of the mice at various time points is displayed in FIG. 39G. As seen in FIG. 39B, treatment with the unedited (WT) iNK cells or the mbIL-15/CD16 (CD16^{+/+}/mbIL-15^{+/+}) DKI iNK cells alone did not lead to tumor reduction in vivo. However, mice treated with iNK cells in combination with trastuzumab exhibited greater tumor reduction than mice treated with trastuzumab alone, whether trastuzumab was administered as a single dose (FIG. 39C) or as a multi-dose regimen (see FIG. 39E). Moreover, the reduction in tumor burden was observed for at least 144 days post-introduction of iNK cells (FIG. 39E). As shown in FIG. 39G, mbIL-15/CD16 (CD16^{+/+}/mbIL-15^{+/+}) DKI iNK cells led to significantly greater in vivo tumor reduction as compared to the unedited (WT) iNK cells measured at day 33. This was seen with two different mbIL-15/CD16 (CD16^{+/+}/mbIL-15^{+/+}) DKI clones (A2 and A4) in combination with a single dose of trastuzumab, or with clone A2 in combination with multi-dose regimen of trastuzumab. Furthermore, treatment with mbIL-15/CD16 (CD16^{+/+}/mbIL-15^{+/+}) DKI iNK cells in combination with a single dose of trastuzumab led to significantly greater in vivo tumor reduction as compared to unedited iNK cells in combination with a single dose of trastuzumab or trastuzumab alone as early as at least day 11 and as late as at least day 54 (FIG. 39H).

Mice treated with mbIL-15/CD16 (CD16^{+/+}/mbIL-15^{+/+}) DKI iNK cells in combination with trastuzumab exhibited significantly prolonged survival as compared to mice treated with trastuzumab alone (FIGS. 39D and 39F). In addition, mice treated with mbIL-15/CD16 (CD16^{+/+}/mbIL-15^{+/+}) DKI iNK cells also exhibited significantly prolonged survival as compared to mice treated with unedited (WT) iNK cells (FIG. 39F). As shown in FIG. 39I, treatment with mbIL-15/CD16 (CD16^{+/+}/mbIL-15^{+/+}) DKI iNK cells in combination with trastuzumab led to complete tumor clearance in multiple animals, with 50% (4/8) of mice being tumor-free at day 40 following treatment with DKI iNK cells in combination with multiple doses of trastuzumab. Continued monitoring revealed that at day 144, 75% (6/8) of the mice treated with mbIL-15/CD16 (CD16^{+/+}/mbIL-15^{+/+}) DKI iNK cells in combination with trastuzumab (×3) showed no detectable tumor by BLI (e.g., were tumor free). Furthermore, histological analysis targeting Her2 (tumor antigen expressed on SKOV3 cells) in the lung tissue of mice revealed that mbIL-15/CD16 (CD16^{+/+}/mbIL-15^{+/+}) DKI iNK cells resulted in reduced metastasis compared with unedited iNK cells and completely inhibited tumor metastasis in 86% of mice, compared with only 14% with unedited iNK cells (data not shown). These results confirm that mbIL-15/CD16 (CD16^{+/+}/mbIL-15^{+/+}) DKI iNK cells readily kill tumor cells in vivo and demonstrate that mbIL-15/CD16 (CD16^{+/+}/mbIL-15^{+/+}) DKI iNK cells in combination with trastuzumab produces greater in vivo tumor reduction than treatment with either trastuzumab alone or with unedited (WT) iNK cells in combination with trastuzumab.

FIG. 39J demonstrates that knock-in of mbIL-15 at an essential gene (the GAPDH locus) prolongs the in vivo persistence of the iNK cells as compared to short-lived WT NK cells. Further, the mbIL-15/CD16 (CD16^{+/+}/mbIL-15^{+/+}) DKI iNK cells continue to express high levels of CD16 on the cell surface due to the knock-in of CD16 at the GAPDH locus (as described in Example 14; bottom right plot of FIG. 39J), indicating that they retain the ability for ADCC mediated tumor killing in the presence of a therapeutic antibody (such as trastuzumab). Meanwhile, WT iNK cells were not detectable at day 144 (top left and top right plots of FIG. 39J). These data demonstrate that mbIL-15/CD16 (CD16^{+/+}/mbIL-15^{+/+}) DKI iNK cells are capable of persisting in vivo and maintaining high CD16 expression up to at least 144 days. This is notable, given that unedited NK cells have been reported to have limited persistence (see, e.g., Romee et al., Sci. Trans. Med. 8:357ra123357ra123 (2016)).

### Example 16: Computation Screening of AsCpf1 Guide RNAs Suitable for Selection by Essential-Gene Knock-In

The present example describes a method for computationally screening for AsCpf1 (AsCas12a; e.g., as represented by SEQ ID NO: 62) guide RNAs (gRNAs) suitable for methods described herein that target a number of essential housekeeping genes. The results of this screening are summarized in Table 20, these gRNAs facilitate Cas12a cleavage within the last 500 bp of the DNA coding sequences for the listed essential genes.

The essential genes in Table 20 selected for this analysis were identified in a pool of essential genes made by combining the essential genes described in Eisenberg et al., (see e.g., Eisenberg and Levanon, Human housekeeping genes, revisited. Trends Genetics, 2014) and the genes described in Yilmaz et al., (see e.g., Yilmaz et al., Defining essential genes for human pluripotent stem cells by CRISPR-Cas9 screening in haploid cells. Nature Cell Biology, 2018). In brief, essential genes described in Yilmaz et al., with CRISPR Scores less than 0, and FDR of <0.05 were combined with essential genes described in Eisenberg & Levanon to create a list of 4,582 genes in total. These genes were then sorted by their average expression level (mean normalized expression across different tissues, see e.g., RNA consensus tissue gene expression data provided by https://www.proteinatlas.org/download/rna_tissue_consensus.tsv.zip), and the 100 genes with the highest average expression levels across tissues were selected for the analysis. GAPDH was present within this group of genes. TBP, E2F4, G6PD and KIF11 were added to this group, making 104 genes in total, for further analysis.

Potential gRNA target sequences for each of the genes of interest were generated by searching for nuclease specific PAMs with suitable protospacers mapped to a representative coding region (mRNA-201). Transcripts with its name followed by “−201” were selected as the representative for each gene (e.g., GAPDH-201). Gene information (i.e., coding region) was obtained from GENCODE v.37 gene annotation GTF file. Potential gRNAs were first searched within the genomic regions of target genes in the human reference genome (hg38), and those identified gRNAs with their cut sites within 500 bp of the representative coding region's stop site were selected for further analysis. The candidate gRNAs were then aligned to the human reference genome (e.g., hg38) with BWA Aln (maximum mismatch tolerance-n 2). Guides with potential off target binding sites (i.e., aligning to multiple genomic regions; mapping quality MAPQ <30) were filtered out. The resultant gRNAs target highly and/or broadly expressed essential genes within 500 coding base pairs of a representative stop-codon and have no identical off-target binding sites annotated in the human genome. Thus, they are excellent candidate gRNAs for the selection methods described herein.

TABLE 20
AsCas12 guide RNAs

| SEQ | | Target Domain Sequence |
|---|---|---|
| ID NO | Gene | (DNA) |
| 2250 | EIF4G2 | AGGCTTTGGCTGGTTCTTTAG |
| 2260 | EIF4G2 | GCTGGTTCTTTAGTCAGCTTC |
| 2270 | EIF4G2 | GTCAGCTTCTTCCTCTGATTC |
| 2280 | EIF4G2 | TAACCAGGTTAGCCACTGATT |
| 2290 | EIF4G2 | ACAAAAGACTTACCTGGAACA |
| 2300 | EIF4G2 | CCGGAAACTCTTGGGTTATAT |
| 2310 | EIF4G2 | CAAGCCAAGAAAGCTTCTTCT |
| 2320 | EIF4G2 | CATGTCATAGAAGTGCACAAA |
| 2330 | EIF4G2 | GGAAGTTGCTGTTATAGCAGT |
| 2340 | EIF4G2 | TGCATTACTGGCTTGAAAGAT |
| 2350 | EIF4G2 | CTGCTCTAACTGTTCTTTGGA |
| 2360 | EIF4G2 | GAAGGAGCAGAGGATGAATCT |
| 2370 | EIF4G2 | ATCGCTGGGGGGGTTTACTTC |
| 2380 | EIF4G2 | CTTCACTAGAAATGTACTGTA |
| 2390 | EIF4G2 | TCTACATGAAGTTTGGGAGAG |
| 2400 | EIF4G2 | GGAGAGATGTTATCTTTAATC |
| 2410 | EIF4G2 | TATATGGTTTGAGGGGATGGA |
| 2420 | EIF4G2 | AGGGGATGGATCCAACTTTAT |
| 2430 | EIF4G2 | TAGGTGAATCAGTGGCTAACC |
| 2440 | EIF4G2 | CAAATCTTAATTTATAGGTGA |
| 2450 | EIF4G2 | ATTTACAAATCTTAATTTATA |
| 2460 | EIF4G2 | CGGGAAAAGGCAAGGCTTTGT |
| 2470 | EIF4G2 | TTGGCTTGGAAAGAAGATATA |
| 2480 | EIF4G2 | TGCACTTCTATGACATGGAAA |
| 2490 | EIF4G2 | AGGCATGTTACTTCGCTTTTT |
| 2500 | EIF4G2 | TTCATGATCACGTTGATCTAC |
| 2510 | EIF4G2 | AAGCCAGTAATGCAGAAATTT |
| 2520 | EIF4G2 | TAGTGAAGTAAACCCCCCCAG |
| 2530 | EIF4G2 | TGTCCAGCTTCTTACAGTACA |
| 2540 | EIF4G2 | TGAACATCTTAATGACTAGGT |
| 2550 | SKP1 | AAGACCTTACCTTTTTTAATA |
| 2560 | SKP1 | CAATGAACTTACCTTCCAACA |
| 2570 | SKP1 | AGCAGGGCAGAATAAAAACCA |
| 2580 | SKP1 | TTCATAATTTCAGCAGGGCAG |
| 2590 | SKP1 | CTTTGTTCATAATTTCAGCAG |
| 2600 | SKP1 | CAGGCTGCAAACTACTTAGAC |
| 2610 | SKP1 | TTGTTGTAGGTCATTCAGTGG |
| 2620 | SKP1 | TTAGATTTGGGAATGGATGAT |
| 2630 | SKP1 | TTCTGGTTTTCTTAGATTTGG |
| 2640 | SKP1 | GATGCCTTCAATTAAGTTGCA |
| 2650 | SKP1 | ATGTCCTTTTTTTTTAGATGC |
| 2660 | RPS3 | AAGCTTTATGCTGAAAAGGTG |
| 2670 | RPS3 | AAGGGCCTGCTATGGTGTGCT |
| 2680 | RPS3 | AAGGAAGCAAGGGATATCCTG |
| 2690 | RPS3 | AGCATAAAGCTTTAAAGGAAG |
| 2700 | RPS3 | CCAGACACCACAACCTCGCAG |
| 2710 | RPS3 | CCAAGCACTCTCAGCTGCTCA |
| 2720 | RPS19 | TTCTTCCATCTTTTCCCACAG |
| 2730 | RPS19 | CCACAGGTGGCAGCTGCCAAC |
| 2740 | RPS19 | TCTGACGTCCCCCATAGATCT |
| 2750 | HMGB1 | AGCCCTCTTACCTTCCACCTC |
| 2760 | HMGB1 | TGTTCATTTATTGAAGTTCTA |
| 2770 | HMGB1 | GTTCGGCCTTCTTCCTCTTCT |
| 2780 | HMGB1 | TAGACCATGTCTGCTAAAGAG |
| 2790 | HMGB1 | GAAAAATAACTAAACATGGGC |
| 2800 | RPL7 | CCCCAAATAGAACCTACCAAG |
| 2810 | RPL7 | ACTTCAGGTACCCCAATCTGA |
| 2820 | RPL7 | CTTTTTCACTTCAGGTACCCC |
| 2830 | RPL7 | TGTTTGCTTTTTCACTTCAGG |
| 2840 | RPL7 | ACCACAGTATCAATGGAGTGA |
| 2850 | RPL7 | TGGTCCGTTTTCACCACAGTA |
| 2860 | RPLP0 | AGGTCAAGGCCTTCTTGGCTG |
| 2870 | RPLP0 | ACCACTTCCCCCCTCCTTTCA |
| 2880 | G6PD | CTCACCTGCCATAAATATAGG |
| 2890 | G6PD | CAGTATGAGGGCACCTACAAG |
| 2900 | G6PD | ACCCCACTGCTGCACCAGATT |
| 2910 | G6PD | CGCCACGTAGGGGTGCCCTTC |
| 2920 | RPL4 | GCTTGTAGTGCCGCTGCTGCA |
| 2930 | RPL4 | CCGTGGTGCTCGAAGGGCTCT |
| 2940 | RPL4 | TTGCAGCACAAGCTCCGGGTG |
| 2950 | RPL4 | TGCCTAATTTGTTGCAGCACA |
| 2960 | RPL4 | TAGCAAGAAGATCCATCGCAG |
| 2970 | RPL4 | AGTCTTCCCATGCACAAGATG |
| 2980 | RPL4 | CCTTTCAGTCTTCCCATGCAC |
| 2990 | EEF1G | TCCCCAGCTGAGTCCAGATTG |
| 3000 | EEF1G | TTCCTCTTAGTACCTTTGTGT |
| 3010 | RPL31 | GATGGCTCCCGCAAAGAAGGG |
| 3020 | RPL31 | AATCGTAGGGGCTTCAAGAAG |
| 3030 | RPL31 | TTAGGAATGTGCCATACCGAA |
| 3040 | RPL31 | CAGATCTACAGACAGTCAATG |
| 3050 | RPL31 | GCACCTTATTCCTTTGGCCCA |
| 3060 | RPL31 | TGGGATGGAGAACTTACTTTT |
| 3070 | RPL31 | ATCTGACGATCAGCGATTAGT |
| 3080 | ITM2B | ACTGTCTTTTTCATATTTTAG |
| 3090 | ITM2B | ATATTTTAGGACCCAGATGAT |
| 3100 | ITM2B | GGACCCAGATGATGTGGTACC |
| 3110 | ITM2B | GACTAGCATTTATGCTTGCAG |
| 3120 | ITM2B | TGCTTGCAGGTGTTATTCTAG |
| 3130 | ITM2B | TGAATGTAGGCTGGAACCTAT |
| 3140 | ITM2B | CCTCAGTCCTATCTGATTCAT |
| 3150 | ITM2B | TTTATTTATCGACTGTGTCAT |
| 3160 | ITM2B | TTTATCGACTGTGTCATGACA |
| 3170 | ITM2B | TCGACTGTGTCATGACAAGGA |
| 3180 | ITM2B | CCTCTCCAACAGGTATTCAGA |
| 3190 | ITM2B | GCAATTCGGCATTTTGAAAAC |
| 3200 | ITM2B | AAAACAAATTTGCCGTGGAAA |
| 3210 | ITM2B | CCGTGGAAACTTTAATTTGTT |
| 3220 | ITM2B | GCCAACTGGTACCACATCATC |
| 3230 | ITM2B | TACAAGTATGCTCCTCCTAGA |
| 3240 | ITM2B | CACTTACTTGAAGTGCAAAAT |
| 3250 | ITM2B | AATGCGATCAGTAATAACCAT |
| 3260 | ITM2B | CTTGTCATGACACAGTCGATA |
| 3270 | ITM2B | TAAGTTTCCTTGTCATGACAC |
| 3280 | ITM2B | TCTGCGTTGCAGTTTGTAAGT |
| 3290 | ITM2B | ATAGTTTCTCTGCGTTGCAGT |
| 3300 | ITM2B | AAAAGTATTACCTTTAATAGT |
| 3310 | ITM2B | ATATTTAAAAAGTATTACCTT |
| 3320 | ITM2B | AAAATGCCGAATTGCGAAACA |
| 3330 | ITM2B | TTTTCAAAATGCCGAATTGCG |
| 3340 | ITM2B | CACGGCAAATTTGTTTTCAAA |
| 3350 | ITM2B | TTGACTGTTCAAGAACAAATT |
| 3360 | RPL23A | CTTTTCTCCCAGCTCCTGCCC |
| 3370 | RPL23A | TCCCAGCTCCTGCCCCTCCTA |
| 3380 | RPL23A | CCTCTCCCAGGCTTGACCACT |
| 3390 | RPL23A | TTTTTCAGATTGGGATCATCT |
| 3400 | RPL23A | TAGGAAGGAAACTTACTTTGT |
| 3410 | RPL27A | GTCTGGGCTGCCAACATGGTA |
| 3420 | RPL27A | TATTCCTGCAGGCAAGCACCG |
| 3430 | RPL27A | TCTGTTCTTCTAGGGCTACTA |
| 3440 | PCBP2 | CCCTCTGACTCTCTCCCAGTC |
| 3450 | PCBP2 | CTCCTTTTGTAGGCCTATACC |
| 3460 | PCBP2 | TAGGCCTATACCATTCAAGGA |
| 3470 | PCBP2 | CTCCTTGCAGTTGACCAAGCT |
| 3480 | PCBP2 | ACTTGTATCTTAACAGGCATT |
| 3490 | PCBP2 | GCAGGTTTGGATGCATCTGCT |
| 3500 | PCBP2 | TTTCTCCCTTAAGTTGATTGG |
| 3510 | PCBP2 | TCCCTTAAGTTGATTGGCTGC |
| 3520 | PCBP2 | TGTGTTACAGGCTTTCCTCGG |
| 3530 | PCBP2 | AGCATGAGCCTGAGGGCTTAC |
| 3540 | PCBP2 | TTACCTGACCACCTGCAAAGA |
| 3550 | PCBP2 | ATCATTACCCCAATAGCCTTT |
| 3560 | HSPA8 | TCTTCCTCAGACTGCTGAGAA |
| 3570 | HSPA8 | CTAGGCCGTTTGAGCAAGGAA |
| 3580 | HSPA8 | TTTCCTAGGCCGTTTGAGCAA |
| 3590 | HNRNPK | ATCAGCACTGAAACCAACCTG |
| 3600 | HNRNPK | AGTTGGCTGGATCTATTATTG |
| 3610 | HNRNPK | AAAAATCTTTTCAGTTGGCTG |
| 3620 | HNRNPK | AATCAGATTATTCCTATGCAG |
| 3630 | HNRNPK | TGTTTTTAGGGTGGCTCCGGA |
| 3640 | HNRNPK | TTTCTGTTTTTAGGGTGGCTC |
| 3650 | HNRNPK | TCTCTAACAGGTTGGTTTCAG |
| 3660 | RPL5 | TCTCTTACTATAGATTGCTTA |
| 3670 | RPL5 | CATTGGTTTCTTGAATAGCTT |
| 3680 | RPL5 | TTGAATAGCTTCTCAATAGGT |
| 3690 | UBL5 | TGTAGCTCCAGCTAGGATGAT |
| 3700 | UBL5 | CCTTAACTGCTCTGCGCCCAG |
| 3710 | UBL5 | TTAGGTACACGATTTTTAAGG |
| 3720 | UBL5 | CTTCAGATGAAATCCACGATG |
| 3730 | CST3 | GACAAGGTCATTGTGCCCTGC |
| 3740 | CST3 | AGATGTGGCTGGTCATGGAAG |
| 3750 | CST3 | TTGTACTCGCCGACGGCAAAG |
| 3760 | CST3 | CAGATCTACGCTGTGCCTTGG |
| 3770 | CST3 | ACAGAAAGCATTCTGCTCTTT |
| 3780 | CST3 | CTTTCACAGAAAGCATTCTGC |
| 3790 | CST3 | ACATGTGTAGATCGTAGCTGG |
| 3800 | CST3 | CCGTCGGCGAGTACAACAAAG |
| 3810 | RPS29 | TCACCAAGAGCGAGAACCCTG |
| 3820 | RPS29 | TTACAGTCGTGTCTGTTCAAA |
| 3830 | RPS10 | TACTGTACATGCTTCCTTTTT |
| 3840 | RPS10 | CAAATGACATTATCTGAGAGC |
| 3850 | RPS10 | CTCACGTGGCACAGCACTCCG |
| 3860 | RPS10 | TGTGGGAACCATACCTTTAGG |
| 3870 | RPS10 | TAAAAAGGAAGCATGTACAGT |
| 3880 | RPS10 | TCCTATGGCAGGTCCTCATAG |
| 3890 | RPS10 | TAGCTGGTGCCGACAAGAAAG |
| 3900 | RPS10 | ACTTTCTAGCTGGTGCCGACA |
| 3910 | RPS10 | CATAGGTCTGGAGGGTGAGCG |
| 3920 | RPS10 | ATTTACATAGGTCTGGAGGGT |
| 3930 | RPS10 | TGCCTTACAGTCTCTCAAGTC |
| 3940 | RPL6 | TTACCAGTCACAAGTAATAAG |
| 3950 | RPL6 | GAAATATGAGATTACGGAGCA |
| 3960 | RPL6 | TTTAGAAATATGAGATTACGG |
| 3970 | RPL6 | TCTTTATTTAGAAATATGAGA |
| 3980 | RPL6 | ATTTTCTCTTTATTTAGAAAT |
| 3990 | RPL6 | CCCCTTAGGACCTCTGGTCCT |
| 4000 | RPL6 | ACTTACAGAGGGTGGTTTTCC |
| 4010 | RPL6 | TTTTTAACTTACAGAGGGTGG |
| 4020 | RPLP2 | TGTAGGTATTGGCAAGCTTGC |
| 4030 | ARF1 | ACACTGGCTGCCCGGCAGGCC |
| 4040 | RPL15 | TGTGTAGGTTACGTTATATAT |
| 4050 | RPL15 | CTATTCTAGGAGCGAGCTGGA |
| 4060 | RPL15 | CCTCTGCAACGGACTGAAGGC |
| 4070 | FAU | CTGGCCGGTCACCTCGAAGGT |
| 4080 | FAU | CCTGTAGGCTCATGTAGCCTC |
| 4090 | FAU | CTCAGTCGCCAATATGCAGCT |
| 4100 | FAU | TTTACTCAGTCGCCAATATGC |
| 4110 | RPL36 | CCCCCTAGCGTCTGACCAAAC |
| 4120 | RPL36 | CCCCGTACGAGCGGCGCGCCA |
| 4130 | NACA | CTAGTATACCTCTTCCTCTTC |
| 4140 | NACA | CTCACCTTGGCTTCCCCAAAA |
| 4150 | NACA | AAATCTTACCTTCCGTGCCTT |
| 4160 | NACA | TCTGTTACAGGAATTAACAAT |
| 4170 | NACA | CCTCTCATCTCTCAGGTCGAT |
| 4180 | NACA | TACCCTGTAGATCGAAGATTT |
| 4190 | NACA | GGCTATGTCCAAACTGGGTCT |
| 4200 | NACA | TCTTCTTTAGGCTATGTCCAA |
| 4210 | NACA | TCTTCTTAGCTGGCGGCAGCA |
| 4220 | PRDX1 | GACATCAGGCTTGATGGTATC |
| 4230 | PRDX1 | CCATGCTAGATGACAGAAGTG |
| 4240 | PRDX1 | TTAAATTCTTCTGCCCTATCA |
| 4250 | PRDX1 | TCTTGCAGTGTGCCCAGCTGG |
| 4260 | PRDX1 | TCATTGATGATAAGGGTATTC |
| 4270 | PRDX1 | CCAGGGGCCTTTTTATCATTG |
| 4280 | PRDX1 | ATCTCTTTTCCCAGGGGCCTT |
| 4290 | PRDX1 | CTTTCATCTCTTTTCCCAGGG |
| 4300 | PRDX1 | GTATCAGACCCGAAGCGCACC |
| 4310 | PRDX1 | CCATAGGGTCAATACACCTAA |
| 4320 | PRDX1 | CCTTTTGCCATAGGGTCAATA |
| 4330 | PRDX1 | AGTGATAGGGCAGAAGAATTT |
| 4340 | PRDX1 | CCCTCTTGACTTCACCTTTGT |
| 4350 | PRDX1 | CCCCCAGGAAAATATGTTGTG |
| 4360 | ALDOA | CCTTCTCGGTCACATACTGGC |
| 4370 | NCL | GCCCAGTCCAAGGTAACTTTA |
| 4380 | NCL | TTTCCATCAATTTCACCGTCT |
| 4390 | NCL | CATCAATTTCACCGTCTTCCA |
| 4400 | NCL | ACCGTCTTCCATGGCCTCCTT |
| 4410 | NCL | GCATCCTCCTCACTGTTGAAG |
| 4420 | NCL | GAGGACCCAGTTTCCCGGTCA |
| 4430 | NCL | CCGGTCAGTAACTATCCTTGC |
| 4440 | NCL | ATGTCTCTTCAGTGGTATCCT |
| 4450 | NCL | ACAAACAGAGTTTTGGATGGC |
| 4460 | NCL | GTGGCAGAGGCCGGGGAGGCT |
| 4470 | NCL | GAGGACGAGGTGGTGGTAGAG |
| 4480 | NCL | TAGACTTCAACAGTGAGGAGG |
| 4490 | NCL | GTTTTGTAGACTTCAACAGTG |
| 4500 | NCL | GTGTTCTAGGTTTGGTTTTGT |
| 4510 | NCL | ATTTGGTGTTCTAGGTTTGGT |
| 4520 | NCL | ACGGCTCCGTTCGGGCAAGGA |
| 4530 | NCL | TCAAAGGCCTGTCTGAGGATA |
| 4540 | NCL | CTTCCCAGAGCCATCCAAAAC |
| 4550 | BTF3 | TAGATGAAAGAAACAATCATG |
| 4560 | BTF3 | CTCTTCTCCCTGACTTTAGGG |
| 4570 | BTF3 | GGGAACTGCTCGCAGAAAGAA |
| 4580 | BTF3 | TTTTCTTAATAGGTGAATATG |
| 4590 | BTF3 | TTAATAGGTGAATATGTTTAC |
| 4600 | BTF3 | CATTTTCCTTTCATAGCTGTG |
| 4610 | BTF3 | CTTTCATAGCTGTGGATGGAA |
| 4620 | BTF3 | ATAGCTGTGGATGGAAAAGCA |
| 4630 | BTF3 | TACTCTTTTCCTTTTCCTAGA |
| 4640 | BTF3 | CTTTTCCTAGATCTTGTGGAG |
| 4650 | BTF3 | CTAGATCTTGTGGAGAATTTT |
| 4660 | BTF3 | ATACTTGCCTCTTCAATACCA |
| 4670 | E2F4 | GGGGCTATCATTGTAGTGAGT |
| 4680 | E2F4 | AGCCCATCAAGGCAGACCCCA |
| 4690 | E2F4 | AGTTTTGGAACTCCCCAAAGA |
| 4700 | E2F4 | GAACTCCCCAAAGAGCTGTCA |
| 4710 | E2F4 | CCCCTCTGCTTCGTCTTTCTC |
| 4720 | E2F4 | TCCACCCCCGGGAGACCACGA |
| 4730 | E2F4 | ATGTGCCTGTTCTCAACCTCT |
| 4740 | E2F4 | TGACAGCTCTTTGGGGAGTTC |
| 4750 | KIF11 | ACTAAGCTTAATTGCTTTCTG |
| 4760 | KIF11 | TGGAACAGGATCTGAAACTGG |
| 4770 | KIF11 | TACCCATCAACACTGGTAAGA |
| 4780 | KIF11 | TTCTTTTAGGATGTGGATGTA |
| 4790 | KIF11 | GGATGTGGATGTAGAAGAGGC |
| 4800 | KIF11 | CCGCCTTAAATCCACAGCATA |
| 4810 | KIF11 | ATTAAGTTCTAGATTTTGTGC |
| 4820 | KIF11 | TGGTTTCATTAAGTTCTAGAT |
| 4830 | KIF11 | AGATCCTGTTCCAGAAAGCAA |
| 4840 | KIF11 | AAGTACCTGTTGGGATATCCA |
| 4850 | KIF11 | TCTTTTAAAGTACCTGTTGGG |
| 4860 | KIF11 | AGCTGATCAAGGAGATGTTCA |
| 4870 | KIF11 | CTTTTCAGCTGATCAAGGAGA |
| 4880 | KIF11 | GCATCATTAACAGCTCAGGCT |
| 4890 | KIF11 | TGAACAGTTTAGCATCATTAA |
| 4900 | KIF11 | TTGTTTTCTGAACAGTTTAGC |
| 4910 | KIF11 | CCGGAATTGTCTCTTCTTTGT |
| 4920 | KIF11 | AATTTACCGGAATTGTCTCTT |
| 4930 | KIF11 | TCTTTTCCATGTGATTTTTTA |
| 4940 | KIF11 | TTTGTCTTTTCCATGTGATTT |
| 4950 | KIF11 | GACCTCTCCAGTGTGTTAATG |
| 4960 | KIF11 | TTCCACTTTAGACCTCTCCAG |
| 4970 | KIF11 | TAACCAAGTGCTCTGTAGTTT |
| 4980 | RPL13 | TCTTCTAGGTCTATAAGAAGG |
| 4990 | RPL13 | AGTAAGTGTTCACTTACGTTC |
| 5000 | PFDN5 | CCTTAATTCTTGCTTCTCAGA |
| 5010 | PFDN5 | AGCTGAGCAATGGACGTGGAC |
| 5020 | PTMA | AAGGACTTAAAGGAGAAGAAG |
| 5030 | PTMA | TGTCGAGGAGAATGAGGAAAA |
| 5040 | PTMA | ATTCTCTCCAGGTGAGGAAGA |
| 5050 | PTMA | TCTGCTTAGGATGACGATGTC |
| 5060 | RPL11 | GCATCCGGAGAAATGAAAAGA |
| 5070 | RPL11 | TCCACAGGTGCGGGAGTATGA |
| 5080 | RPL11 | AGCATCGCAGACAAGAAGCGC |
| 5090 | RPL11 | AGTATGATGGGATCATCCTTC |
| 5100 | RPL11 | CGGATGCGAAGTTCCCGCATG |
| 5110 | RPL11 | TCCGGATGCCAAAGGATCTGA |
| 5120 | RPL11 | ATTTCTCCGGATGCCAAAGGA |
| 5130 | RPL11 | GACCCTTCTCCAAGATTTCTT |
| 5140 | RPL11 | TTAACTCATACTCCCGCACCT |
| 5150 | RPL11 | CCTTCTGCTGGAACCAGCGCA |
| 5160 | COX7C | TCTTTTTTTCCAACAGAATTT |
| 5170 | COX7C | CAACAGAATTTGCCATTTTCA |
| 5180 | RPL8 | TTGAGGCCCTCAGCACTAGTT |
| 5190 | RPL8 | CGGCCAGCAGGGGCATCTCTG |
| 5200 | RPL8 | TGGGTTACTTACATTCATGGC |
| 5210 | RPL8 | TCTGCCTGCAGCCTGTGGAGC |
| 5220 | RPL10 | TTCTCCCTACCTAGCCCTGGA |
| 5230 | RPL10 | CATTGCTCCTTAGATCCACAT |
| 5240 | RPL32 | CCTCCCCAAAAGGAAGAGTTC |
| 5250 | TBP | CTGCGGTAATCATGAGGATAA |
| 5260 | TBP | AGTTCTGGGAAAATGGTGTGC |
| 5270 | TBP | CTTTCCCTAGTGAAGAACAGT |
| 5280 | TBP | CCTAGTGAAGAACAGTCCAGA |
| 5290 | TBP | CAGCTAAGTTCTTGGACTTCA |
| 5300 | TBP | CTATAAGGTTAGAAGGCCTTG |
| 5310 | TBP | CAATTTTCCTTCTAGTTATGA |
| 5320 | TBP | CTTCTAGTTATGAGCCAGAGT |
| 5330 | TBP | CTGGTTTAATCTACAGAATGA |
| 5340 | TBP | ATCTACAGAATGATCAAACCC |
| 5350 | TBP | TTTCTGGAAAAGTTGTATTAA |
| 5360 | TBP | TGGAAAAGTTGTATTAACAGG |
| 5370 | TBP | GGTCAAGTTTACAACCAAGAT |
| 5380 | TBP | GGGCACGAAGTGCAATGGTCT |
| 5390 | TBP | CCAGAACTGAAAATCAGTGCC |
| 5400 | TBP | TTACGGCTACCTCTTGGCTCC |
| 5410 | TBP | TTGCTGCCAGTCTGGACTGTT |
| 5420 | TBP | AGACTTACCTACTAAATTGTT |
| 5430 | TBP | ATCATTCTGTAGATTAAACCA |
| 5440 | TBP | CAGAAACAAAAATAAGGAGAA |
| 5450 | TBP | AAATGCTTCATAAATTTCTGC |
| 5460 | CD63 | CTCAGCCAGCCCCCAATCTTC |
| 5470 | CD63 | TCCCAATCTGTGTAGTTAGCA |
| 5480 | CD63 | GGGTAATTCTCCATCTGCTGC |
| 5490 | CD63 | GGAATTGTCTTTGCCTGCTGC |
| 5500 | CD63 | CTTCTAGGTTTTGGGAATTGT |
| 5510 | CD63 | TGCCTGCCACCTTCAGGGCTG |
| 5520 | CD63 | AACGAGAAGGCGATCCATAAG |
| 5530 | CD63 | AGTGCTGTGGGGCTGCTAACT |
| 5540 | CD63 | TTCCCTCCCCCAGTTTAAGTG |
| 5550 | CD63 | ATAACAACTTCCGGCAGCAGA |
| 5560 | CD63 | TGTCTCTTATCATGTTGGTGG |
| 5570 | CD63 | CCATCTTTCTGTCTCTTATCA |
| 5580 | CD63 | CTCCTGCAGTTTGCCATCTTT |
| 5590 | CD63 | TGGGCTGCTGCGGGGCCTGCA |
| 5600 | RPS24 | TGTTTTCAGAACGACACCGTA |
| 5610 | RPS24 | AGAACGACACCGTAACTATCC |
| 5620 | RPS24 | GGTCATTGATGTCCTTCACCC |
| 5630 | RPS24 | TCATTCAGCATGGCCTGTATG |
| 5640 | RPS24 | CCTCTTCTTCTGGATTACAGA |
| 5650 | RPS24 | TAGTGCGGATAGTTACGGTGT |
| 5660 | RPS24 | CTTAATGAACTATACCTTTTT |
| 5670 | RPS23 | GGGCTGTGCCCAAATGAGCTT |
| 5680 | RPS23 | TTCCAGGAAAATGATGAAGTT |
| 5690 | RPS23 | TACCCAATGACGGTTGCTTGA |
| 5700 | RPS23 | AGAGGAGTTGAAGCCAAACAG |
| 5710 | RPS23 | TATTTCAGAGGAGTTGAAGCC |
| 5720 | RPS23 | GGCAAGTGTCGTGGACTTCGT |
| 5730 | RPS23 | ATTTTTAGGCAAGTGTCGTGG |
| 5740 | EEF2 | TCCAGGAAGTTGTCCAGGGCA |
| 5750 | EEF2 | AGGCCCTTGCGCTTGCGGGTC |
| 5760 | EEF2 | ACCACTGGCAGATCCTGCCCG |
| 5770 | EEF2 | TGGTCAAGGCCTATCTGCCCG |
| 5780 | EEF2 | AACAGGAAGCGGGGCCACGTG |
| 5790 | EEF2 | CCTTCTGGCAGTGTCCAGAGC |
| 5800 | EEF2 | TTTCCCTTCTGGCAGTGTCCA |
| 5810 | CALR | CTTCTCCCTTCTGCAGGGTGA |
| 5820 | CALR | GCGTGCTGGGCCTGGACCTCT |
| 5830 | CALR | ACAACTTCCTCATCACCAACG |
| 5840 | CALR | GCAACGAGACGTGGGGCGTAA |
| 5850 | CALR | TGGGTGGATCCAAGTGCCCTT |
| 5860 | CALR | CTCCAAGTCTCACCTGCCAGA |
| 5870 | CALR | TTACGCCCCACGTCTCGTTGC |
| 5880 | CALR | TCCTTCATTTGTTTCTCTGCT |
| 5890 | CALR | TTGTCTTCTTCCTCCTCCTTA |
| 5900 | CALR | TCCTCATCATCCTCCTTGTCC |
| 5910 | RPL36AL | TATGCCCAGGGAAGGAGGCGC |
| 5920 | SRP14 | AGGCTTATTCAAACCTCCTTA |
| 5930 | SRP14 | AGGTGAGCTCCAAGGAAGTGA |
| 5940 | SRP14 | CTTCTTTTTCAGGTGAGCTCC |
| 5950 | SRP14 | CTTCAGATGACGGTCGAACCA |
| 5960 | SRP14 | CAGAAGTGCCGGACGTCGGGC |
| 5970 | SRP14 | CAGTTCCTGACGGAGCTGACC |
| 5980 | GABARAP | TTTCGGATCTTCTCGCCCTCA |
| 5990 | GABARAP | GGATCTTCTCGCCCTCAGAGC |
| 6000 | GABARAP | TCTACATTGCCTACAGTGACG |
| 6010 | GABARAP | ATCCCAGGAACACCATGAAGA |
| 6020 | GABARAP | TGCTTTCATCCCAGGAACACC |
| 6030 | GABARAP | TCAACAATGTCATTCCACCCA |
| 6040 | GABARAP | TTTGTCAACAATGTCATTCCA |
| 6050 | GABARAP | CAGTTGGTCAGTTCTACTTCT |
| 6060 | GABARAP | TTGCATCTTGTATCTTTTGCA |
| 6070 | GABARAP | TCAGGTGATAGTAGAAAAGGC |
| 6080 | GABARAP | ATCTCTTTATCAGGTGATAGT |
| 6090 | RPSA | ATAATCTGCCACTCTTGGCAG |
| 6100 | RPSA | TAACCCAGATTGAAAAAGAAG |
| 6110 | RPSA | GTATTCTCTTAACAGAAGACT |
| 6120 | RPSA | GAGAAGCTTACCTCTTCAGGA |
| 6130 | SET | AATTATTTATTACAGTATTTT |
| 6140 | SET | TTACAGTATTTTGATGAAAAT |
| 6150 | SET | GGATTTGACGAAACGTTCGAG |
| 6160 | SET | ACGAAACGTTCGAGTCAAACG |
| 6170 | SET | AGGTTCCCGATATGGATGATG |
| 6180 | SET | TTTCAGGAGGATGAAGGAGAA |
| 6190 | SET | AGGAGGATGAAGGAGAAGATG |
| 6200 | SET | TTTTACCTCTCCTTCCTCCCC |
| 6210 | SET | GCCAAATTTTCTTTTACCTCT |
| 6220 | GAPDH | CAGACCACAGTCCATGCCATC |
| 6230 | GAPDH | ATCTTCTAGGTATGACAACGA |
| 6240 | RPLP1 | TTTGTTGTAGGAGGATAAGAT |
| 6250 | RPLP1 | TTGTAGGAGGATAAGATCAAT |
| 6260 | RPLP1 | TAGCTGAGGAGAAGAAAGTGG |
| 6270 | RPLP1 | CCACCATCACCTTACCTTTGC |
| 6280 | RPLP1 | CTACCTGGAGCAGCAGCAGTG |
| 6290 | CFL1 | CTCTTAAGGGGCGCAGACTCG |
| 6300 | CFL1 | TAGGGATCAAGCATGAATTGC |
| 6310 | CFL1 | TTCTTTATAGGGATCAAGCAT |
| 6320 | CFL1 | TGTCCAGGGCCCCCGAGTCTG |
| 6330 | RPS15 | CTCTTGGTCTCCCGCAGCCCG |
| 6340 | TPT1 | CATTATTTATTTTAACCCACT |
| 6350 | TPT1 | TTTTAACCCACTTCCTTGTAC |
| 6360 | TPT1 | ACCCACTTCCTTGTACTTACA |
| 6370 | TPT1 | CCTGGTAGTTTTTGAAATTAG |
| 6380 | TPT1 | GAAATGGAAAAATGTGTAAGT |
| 6390 | TPT1 | CTTCCCAAGTTCTTTATTGGT |
| 6400 | TPT1 | TTTGCTTCCCAAGTTCTTTAT |
| 6410 | TPT1 | GAATCAAAGGGAAACTTGAAG |
| 6420 | TPT1 | TTAATGCAGATGGTCAGTAGG |
| 6430 | RPL23 | CTACCTTTCATCTCGCCTTTA |
| 6440 | RPL23 | TTGTTCACTATGACTCCTGCA |
| 6450 | RPL23 | CTCACCCTTTTTTCTGAGCTC |
| 6460 | RPL23 | ATGCAGGTTCTGCCATTACAG |
| 6470 | RPL23 | TTTTTTTAATGCAGGTTCTGC |
| 6480 | RPL23 | TTCTCTCAGTACATCCAGCAG |
| 6490 | RPL34 | ACTTTCTAGGTCCCGAACCCC |
| 6500 | RPL34 | TAGGTCCCGAACCCCTGGTAA |
| 6510 | RPL34 | TTATGCAGGTTCGTGCTGTAA |
| 6520 | RPL34 | GTATTTTCCTTTCTAGGATCA |
| 6530 | RPL34 | CTTTCTAGGATCAAGCGTGCT |
| 6540 | RPL34 | TAGGATCAAGCGTGCTTTCCT |
| 6550 | RPL34 | AGAAATACTTACAGCCTAGTT |
| 6560 | RPL34 | ACTTACCTGTCACGAACACAT |
| 6570 | RPL34 | AGCATTTAACTTACCTGTCAC |
| 6580 | COX4I1 | TCTTTCAGAATGTTGGCTACC |
| 6590 | COX4I1 | AGAATGTTGGCTACCAGGGTA |
| 6600 | COX4I1 | CACCTCTGTGTGTGTACGAGC |
| 6610 | COX4I1 | TTCAATATGTTTTTCAGAAAG |
| 6620 | COX411 | AGAAAGTGTTGTGAAGAGCGA |
| 6630 | COX411 | GCTCCCAGCTTATATGGATCG |
| 6640 | COX411 | CTGAGATGAACAGGGGCTCGA |
| 6650 | COX4I1 | ACCGCGCTCGTTATCATGTGG |
| 6660 | COX4I1 | ACAAAGAGTGGGTGGCCAAGC |
| 6670 | COX4I1 | TCAAAGCTTTGCGGGAGGGGG |
| 6680 | COX4I1 | GTAGTCCCACTTGGAGGCTAA |
| 6690 | RPL27 | TCCTTGCTCTCTGCAGAAATG |
| 6700 | RPL27 | GAACATTGATGATGGCACCTC |
| 6710 | RPL27 | TCCCCAGGTACTCTGTGGATA |
| 6720 | RPL27 | CCTTCTAGATACAAGACAGGC |
| 6730 | RPL27 | CGTCCGGAGTAGCGTCCAGCC |
| 6740 | RPL27 | TCTTTGATCTCTTGGCGATCT |
| 6750 | RPL27 | ACAAAAGATTTTATCTTTGAT |
| 6760 | EDF1 | GAGGCTTTGTGTTCATTTCGC |
| 6770 | EDF1 | TGTTCATTTCGCCCTAGGCCC |
| 6780 | EDF1 | GCCCTAGGCCCCTTCTCGATG |
| 6790 | EDF1 | CAATGTCCTTTCCCCGGAGCT |
| 6800 | EDF1 | CCAAGCACCTGGTTATTGGGT |
| 6810 | EDF1 | TTGGAAGTCTCCACATCTTCT |
| 6820 | EDF1 | GCCTGGGCGGCCGTAGGGCCC |
| 6830 | EDF1 | AGGCCTCAAGCTCCGGGGAAA |
| 6840 | EDF1 | GAAAATCAATGAGAAGCCACA |
| 6850 | EDF1 | CCTCACACCGACTCCAGGGGC |
| 6860 | EDF1 | TAGGCTATCTTAGCGGCACAG |
| 6870 | EDF1 | TAATTTTCTAGGCTATCTTAG |
| 6880 | TMEM59 | AAAGAAAAATGCTTAAATTTC |
| 6890 | TMEM59 | AGAATGAGCAAGATTCACTTT |
| 6900 | TMEM59 | TAGGTAGAGGCCCTGCTTCTT |
| 6910 | TMEM59 | GATCTAACAACCACAAGAGAA |
| 6920 | TMEM59 | GCTTTTGTTCATTCATAAACT |
| 6930 | TMEM59 | TTCATTCATAAACTCCAAGTC |
| 6940 | TMEM59 | CCTCAGAGGGAACATACTGCT |
| 6950 | TMEM59 | TCCATCTTCAAGAAAATTCCT |
| 6960 | TMEM59 | CTTAGAGATGATTCTCTCAAA |
| 6970 | TMEM59 | TAGGCTCCTGCTCCAAATGTG |
| 6980 | TMEM59 | CGTCATCGGCTTGAAGATAAA |
| 6990 | TMEM59 | TGAATGAACAAAAGCTAAACA |
| 7000 | TMEM59 | CAGAAGCTGAGTATCTATGGT |
| 7010 | TMEM59 | TTTTGCAGAAGCTGAGTATCT |
| 7020 | TMEM59 | TTGTGCAACTGTTGCTACAGC |
| 7030 | TMEM59 | GATTTGTTGTGCAACTGTTGC |
| 7040 | TMEM59 | ACTACAACTCTTGTCCTCTCG |
| 7050 | TMEM59 | CAGTAACTCTGGGTGGATTTT |
| 7060 | TMEM59 | TTGAAGATGGAGAAAGTGATG |
| 7070 | TMEM59 | AGCAGATCTGCAAATGAGAAA |
| 7080 | TMEM59 | AGAGAATCATCTCTAAGCAAA |
| 7090 | TMEM59 | GAGCAGGAGCCTACAAATTTG |
| 7100 | TMEM59 | GTCTAAGCCAGAAATCCAGTA |
| 7110 | TMEM59 | ATTATTATTTTAGTCTAAGCC |
| 7120 | TMEM59 | TCTTCAAGCCGATGACGGAAA |
| 7130 | DYNLL1 | TCTTTTCCAGGAATTTGACAA |
| 7140 | DYNLL1 | CAGGAATTTGACAAGAAGTAC |
| 7150 | DYNLL1 | ATGTGTCACATAACTACCGAA |
| 7160 | NME2 | TTTCTTAGGAACATCATTCAT |
| 7170 | NME2 | TTAGGAACATCATTCATGGCA |
| 7180 | TMBIM6 | GCTGATGGCAACACCTCATAG |
| 7190 | TMBIM6 | TGTTTTCTAGGAGTTGGCCTG |
| 7200 | TMBIM6 | TAGGAGTTGGCCTGGGCCCTG |
| 7210 | TMBIM6 | TATTGCTGTCAACCCCAGGTA |
| 7220 | TMBIM6 | TAACAGCATCCTTCCCACTGC |
| 7230 | TMBIM6 | ATGGGCACGGCAATGATCTTT |
| 7240 | TMBIM6 | CCTGCTTCACCCTCAGTGCAC |
| 7250 | TMBIM6 | CTGTGTCTTATAGGTATCTTG |
| 7260 | TMBIM6 | TCTTCCCTGGGGAATGTTTTC |
| 7270 | TMBIM6 | GATCCATTTGGCTTTTCCAGG |
| 7280 | TMBIM6 | TTAGGCAAACCTGTATGTGGG |
| 7290 | TMBIM6 | ATACTCAACTCATTATTGAAA |
| 7300 | TMBIM6 | AGGCACTGCATTGATCTCTTC |
| 7310 | TMBIM6 | ATTACTGTCTTCAGAAAACTC |
| 7320 | TMBIM6 | TCCATTTCTAGGATAAGAAGA |
| 7330 | TMBIM6 | TAGGATAAGAAGAAAGAGAAG |
| 7340 | TMBIM6 | ATGGCTATGAGGTGTTGCCAT |
| 7350 | TMBIM6 | TGTTCAGTTTCATGGCTATGA |
| 7360 | TMBIM6 | CCAGTTCACACTTACCTCCCA |
| 7370 | TMBIM6 | AATAATGAGTTGAGTATCAAA |
| 7380 | TMBIM6 | TGAAGACAGTAATGAAATCTA |
| 7390 | TMBIM6 | ATTCATGGCCAGGATCATCAT |
| 7400 | TMBIM6 | GGTTGTAGGCTAACTAACCTT |
| 7410 | RPS7 | TTTAGGAAATTGAAGTTGGTG |
| 7420 | RPS7 | GGAAATTGAAGTTGGTGGTGG |
| 7430 | RPS7 | CCTTACAGAGGAGAATTCTGC |
| 7440 | RPS7 | AACTATTCTTTTAGCCGTACT |
| 7450 | RPS7 | GCCGTACTCTGACAGCTGTGC |
| 7460 | RPS7 | TTTTCTTGTAGGTTGAAACTT |
| 7470 | RPS7 | TTGTAGGTTGAAACTTTTTCT |
| 7480 | RPS7 | TGAAACTACTAAAATACTCAC |
| 7490 | ACTB | CTTCCCAGGGCGTGATGGTGG |
| 7500 | NPM1 | ATTTGTAGTGATGATGATGAT |
| 7510 | NPM1 | TAATTGCAGTCTATACGAGAT |
| 7520 | NPM1 | GAAATTCATTTCTTTTTCAGG |
| 7530 | NPM1 | TTTTTCAGGGACAAGAATCCT |
| 7540 | NPM1 | AGGGACAAGAATCCTTCAAGA |
| 7550 | NPM1 | TCTTAATAGGGTGGTTCTCTT |
| 7560 | NPM1 | CAGGCTATTCAAGATCTCTGG |
| 7570 | NPM1 | TAAAATCATACTTACTCTTCA |
| 7580 | NPM1 | CTCACTTTTTCTATACTTGCT |
| 7590 | RPS6 | TTTTTCTTGGTACGCTGCTTC |
| 7600 | RPS6 | GGGCCCAGGCGGCGAGGCACT |
| 7610 | RPS6 | GGAGGCTAAGGAGAAGCGCCA |
| 7620 | RPS6 | TTTAGGAGGCTAAGGAGAAGC |
| 7630 | RPS6 | TTTTGTTTAGGAGGCTAAGGA |
| 7640 | RPS6 | GGTAAGAAACCTAGGACCAAA |
| 7650 | RPS6 | AATTTTTAGGTAAGAAACCTA |
| 7660 | RPS6 | TTCTAAGGAGAGAAGGATATT |
| 7670 | RPL12 | CTTAAAGGAACCATTAAAGAG |
| 7680 | RPL12 | TTTACTTAAAGGAACCATTAA |
| 7690 | RPL12 | CTCTTCTGCAGTTAAACACAG |
| 7700 | RPL12 | CTGTTTCCTCTTCTGCAGTTA |
| 7710 | RPL12 | TAGTCTCCAAAAAAAGTTGGT |
| 7720 | RPL12 | TTTCTAGTCTCCAAAAAAAGT |
| 7730 | RPL12 | CCCCAGTATACCTGAGGTGCA |
| 7740 | CAPNS1 | AACCTGTTACCCACAGACCCT |
| 7750 | CAPNS1 | GCATTGACACATGTCGCAGCA |
| 7760 | CAPNS1 | AGGAATTCAAGTACTTGTGGA |
| 7770 | CAPNS1 | CAGTAGTGAACTCCCAGGTGC |
| 7780 | CAPNS1 | ATGTTGTTCCACAAGTACTTG |
| 7790 | CAPNS1 | TACACACCTGCCACCTTTTGA |
| 7800 | CAPNS1 | AGAGGTTTCTACACACCTGCC |
| 7810 | CAPNS1 | ATCTGAGTAGCGTCGGATGAT |
| 7820 | CAPNS1 | TCAAGAGATTTGAAGGCACCT |
| 7830 | CAPNS1 | TCCAGTGCCATCTTTGTCAAG |
| 7840 | RPL3 | CAGGGTGGCTTTGTCCACTAT |
| 7850 | RPS13 | TTTATTAGCTTACCTTTCTGT |
| 7860 | RPS13 | TTAGCTTACCTTTCTGTTCCT |
| 7870 | RPS13 | AGTGAATCATCTACAGCCTCT |
| 7880 | RPS13 | TTTTTCAGTGAATCATCTACA |
| 7890 | RPS13 | CCCTTTTTTCTTTTTCAGTGA |
| 7900 | RPS13 | AGGTGTAATCCTGAGAGATTC |
| 7910 | RPS13 | TATTCCATAACAGTGGTTGAA |
| 7920 | RPS21 | TCCACAGCTCCGCTAGCAATC |
| 7930 | RPS21 | TGACCCTTCTTCTCTTTCTAG |
| 7940 | RPS21 | TAGGTTGACAAGGTCACAGGC |
| 7950 | RPS21 | TTAAGGGTGAGTCAGATGATT |
| 7960 | RPS21 | CCCTGGTTCTAGGAACTTTTG |
| 7970 | RPS21 | AGACGATGCCATCGGCCTTGG |
| 7980 | SERF2 | ATTTTCTTTCCTTAGGCGGTA |
| 7990 | SERF2 | TTTCCTTAGGCGGTAACCAGC |
| 8000 | SERF2 | CTTAGGCGGTAACCAGCGTGA |
| 8010 | SERF2 | TGCTGCCGCCCGCAAGCAGAG |
| 8020 | SERF2 | ATATTCTTCTGGCGGGCGAGC |
| 8030 | SERF2 | CCTTAACCGAGTCGCTCTGCT |
| 8040 | SERF2 | CCTCCCCTCCCTGGGGCTACC |
| 8050 | RPL7A | TTTCCCCTCCTGCCTTTTAGG |
| 8060 | RPL7A | CCCTCCTGCCTTTTAGGGAAG |
| 8070 | RPL7A | GGGAAGACAAAGGCGCTTTGG |
| 8080 | RPL7A | TCTTTTCAGATCCGCCGTCAC |
| 8090 | RPL7A | AGATCCGCCGTCACTGGGGTG |
| 8100 | RPL7A | GGGCCAGGCTGTGTACTTACG |
| 8110 | RPL7A | GTGTAAAGCTGCCTCTTACCT |
| 8120 | HNRNPA2B1 | TAAATTACCTCCACCATATGG |
| 8130 | HNRNPA2B1 | CACTCTTCATTGGACCGTAGT |
| 8140 | HNRNPA2B1 | CAAAATCATTGTAATTTCCAC |
| 8150 | HNRNPA2B1 | TTACCTCCTCCATAGTTGTCA |
| 8160 | HNRNPA2B1 | CACCGCCACCACGTGAATCCC |
| 8170 | HNRNPA2B1 | GTGGTAGCAGGAACATGGGGG |
| 8180 | HNRNPA2B1 | GAAATTATAACCAGCAACCTT |
| 8190 | HNRNPA2B1 | ATAGGAAATTATGGAAGTGGA |
| 8200 | HNRNPA2B1 | GAGGTAGCCCCGGTTATGGAG |
| 8210 | HNRNPA2B1 | TAATAGGTGGCAATTTTGGAG |
| 8220 | HNRNPA2B1 | GGGATGGCTATAATGGGTATG |
| 8230 | HNRNPA2B1 | GCCCCTAACAGATGGATATGG |
| 8240 | HNRNPA2B1 | GGACCAGGACCAGGAAGTAAC |
| 8250 | HNRNPA2B1 | GGGATTCACGTGGTGGCGGTG |
| 8260 | HNRNPA2B1 | GCTTTGGGGATTCACGTGGTG |
| 8270 | HNRNPA2B1 | TTGTAGGCAACTTTGGCTTTG |
| 8280 | HNRNPA2B1 | TCTAGACAAGAAATGCAGGAA |
| 8290 | RPL13A | TCTAACAGAAAAAGCGGATGG |
| 8300 | RPL13A | GCATAGCTCACCTTGTCGTAG |
| 8310 | ENO1 | AGCAGGAGGCAGTTGCAGGAC |
| 8320 | ENO1 | TCCTTCCCAAGAATTGAAGAG |
| 8330 | ENO1 | CCTTTCTCCTTCCCAAGAATT |
| 8340 | ENO1 | TCCTAGATCAAGACTGGTGCC |
| 8350 | ENO1 | TTTTCTCCTAGATCAAGACTG |
| 8360 | ENO1 | CTTAGTGGTGTCTATCGAAGA |
| 8370 | PPIA | CTATATGTTGACAGGGTGGTG |
| 8380 | PPIA | AAGGTTGGATGGCAAGCATGT |
| 8390 | CD81 | CCTGTGAGGTGGCCGCCGGCA |
| 8400 | CD81 | ACCACCTCAGTGCTCAAGAAC |
| 8410 | CD81 | TGTCCCTCGGGCAGCAACATC |
| 8420 | RPL35 | TTGACAATGCGCCCCTCAGGC |
| 8430 | RPL35 | TAGCCGAGTCGTCCGGAAATC |
| 8440 | DAD1 | TTCTGTGGGTTGATCTGTATT |
| 8450 | DAD1 | CCAGCACCATCCTGCACCTTG |
| 8460 | DAD1 | TCTTTGCCAGCACCATCCTGC |
| 8470 | DAD1 | CTGATTTTCTCTTTGCCAGCA |
| 8480 | DAD1 | CAAGGCATCTCCCCAGAGCGA |
| 8490 | DAD1 | CCTGAGAATACAGATCAACCC |
| 8500 | DAD1 | CTTCTTGTGCAGTTTGCCTGA |
| 8510 | DAD1 | TGTTTTGCTTCTTGTGCAGTT |
| 8520 | DAD1 | TCTCGGGCTTCATCTCTTGTG |
| 8530 | DAD1 | GCGGTTCTTAGAAGAGTACTT |
| 8540 | UBA52 | TGAAGACCCTCACTGGCAAAA |
| 8550 | UBA52 | CCAGTGAGGGTCTTCACAAAG |
| 8560 | UBA52 | TGGGCAAGCTGGCGGAGAGAA |
| 8570 | UBA52 | ACCTTCTTCTTGGGACGCAGG |
| 8580 | RPL30 | TAGGTGAAAAGGTTTACTTTT |
| 8590 | RPL30 | TGATTTAAAAAGCATACCTGG |
| 8600 | RPL30 | AAAAGCATACCTGGATCAATG |
| 8610 | RPL30 | GGTGACTCTGACATCATTAGA |
| 8620 | RPL30 | TTTTTTAGGTGACTCTGACAT |
| 8630 | RPL30 | TTTTTATTTTTTAGGTGACTC |
| 8640 | RPL30 | GTTCCCAAAGGAAATCTGAAA |
| 8650 | RPL30 | CCCATTTTGGTTCCCAAAGGA |
| 8660 | RPL30 | TAGAAAAAGTCGCTGGAGTCG |
| 8670 | RPL30 | CTTTGTAGAAAAAGTCGCTGG |
| 8680 | RPL30 | ATGTTTGCTTTGTAGAAAAAG |
| 8690 | RNASEK | CGCCTGCCGCCCCCGGATGGG |
| 8700 | RNASEK | TCCCACCGCTTTCCGAGCCCG |
| 8710 | RNASEK | CGAGCCCGCTTGCACCTCGGC |
| 8720 | RNASEK | TGGCGTCGCTCCTGTGCTGTG |
| 8730 | RPL38 | TGTTGCAGCCTCGGAAAATTG |
| 8740 | RPL38 | TCTCTTTCCCTCTAGGTTTGG |
| 8750 | RPL38 | CCTCTAGGTTTGGCAGTGAAG |
| 8760 | RPL38 | GTCGGGCTGTGAGCAGGAAGT |
| 8770 | MYL12B | TTCTTTCTATTGTCTTCCAGG |
| 8780 | MYL12B | TATTGTCTTCCAGGCACCATT |
| 8790 | MYL12B | GCTAAAGTTCTTTCAGTCATC |
| 8800 | PFN1 | CCCATCAGCAGGACTAGCGCT |
| 8810 | PFN1 | CTCCTCCTCCAGCGCTAGTCC |
| 8820 | PFN1 | TCTTTCCTCCTCCTCCAGCGC |
| 8830 | PFN1 | GCATGGATCTTCGTACCAAGA |
| 8840 | RPS11 | TCCTCATAATCTGTAGACTGA |
| 8850 | RPS11 | TCTTTCCTATCCTTTCAGGCT |
| 8860 | RPS11 | CTATCCTTTCAGGCTATTGAG |
| 8870 | RPS11 | AGGCTATTGAGGGCACCTACA |
| 8880 | RPS11 | TTCTGAGGTTCCCCGCACCTC |

### Example 17: Computation Screening of Guide RNAs for Selection by Essential-Gene Knock-In

The present example describes a method for computationally screening for gRNAs more likely to be suitable for use in targeting essential genes using the selection methods herein that are relevant for different RNA-guided nucleases and variants thereof (e.g., variants of Cas12a, such as Mad7), so long as the RNA-guided nucleases exhibit high cutting efficiency. Cas12b, Cas12e, Cas-Phi, Mad7, and SpyCas9 gRNAs targeting essential genes described preceding examples (GAPDH, TBP, E2F4, G6PD, and KIF11) were selected for this analysis, but a similar process could be applied to identify gRNAs for these RNA-guided nucleases in other essential genes as well. The results of this screening are summarized in Tables 21-25, these gRNAs facilitate DNA cleavage within the last 500 bp of the coding sequences of the listed essential genes.

Potential target sequences for each of the essential genes in this analysis (GAPDH, TBP, E2F4, G6PD, and KIF11) were generated by searching for nuclease specific PAMs (ATTN, TTCN, TTN, TTN, and NGG for Cas12b, Cas12e, CasΦ, Mad7, and SpyCas9 respectively) with suitable protospacers mapped to a representative coding region (mRNA-201). Transcripts with its name followed by “−201” were selected as the representative for each gene (e.g., GAPDH-201). Gene information (i.e., coding region) was obtained from GENCODE v.37 gene annotation GTF file. Potential gRNAs were first searched within the genomic regions of target genes in the human reference genome (hg38), and those identified gRNAs with their cut sites within 500 bp of the representative coding region stop site were selected for further analysis. The candidate gRNAs were then aligned to the human reference genome (e.g., hg38) with BWA Aln (maximum mismatch tolerance-n 2). Guides with potential off target binding sites (i.e., aligning to multiple genomic regions; mapping quality MAPQ<30) were filtered out. The resultant gRNAs target essential genes within 500 coding base pairs of a representative stop-codon and have no identical off-target binding sites annotated in the human genome. Thus, gRNAs in Tables 21-25, corresponding to SEQ ID NOs: 8890-18850, represent excellent candidate gRNAs for applying the selection methods described herein to GAPDH, TBP, E2F4, G6PD, and KIF11.

TABLE 21
Cas12b guide RNAs

| SEQ | | |
|---|---|---|
| ID NO | Gene | Target Domain Sequence (DNA) |
| 8890 | GAPDH | CCCAGCTCTCATACCATGAGTCC |
| 8900 | TBP | TATCCACAGTGAATCTTGGTTGT |
| 8910 | TBP | CACTTCGTGCCCGAAACGCCGAA |
| 8920 | TBP | TCTCTGACCATTGTAGCGGTTTG |
| 8930 | TBP | TAGCGGTTTGCTGCGGTAATCAT |
| 8940 | TBP | TCAGTTCTGGGAAAATGGTGTGC |
| 8950 | TBP | AGAATATGGTGGGGAGCTGTGAT |
| 8960 | TBP | TCCTTCTAGTTATGAGCCAGAGT |
| 8970 | TBP | CCTGGTTTAATCTACAGAATGAT |
| 8980 | TBP | TTCTCCTTATTTTTGTTTCTGGA |
| 8990 | TBP | TTGTTTCTGGAAAAGTTGTATTA |
| 9000 | TBP | ATGAAGCATTTGAAAACATCTAC |
| 9010 | TBP | TAAAGGGATTCAGGAAGACGACG |
| 9020 | TBP | GGCGTTTCGGGCACGAAGTGCAA |
| 9030 | TBP | TATTCGGCGTTTCGGGCACGAAG |
| 9040 | TBP | AAATAGATCTAACCTTGGGATTA |
| 9050 | TBP | TCCCAGAACTGAAAATCAGTGCC |
| 9060 | TBP | CTTACGGCTACCTCTTGGCTCCT |
| 9070 | TBP | TCTTGCTGCCAGTCTGGACTGTT |
| 9080 | TBP | TGAATCTTGAAGTCCAAGAACTT |
| 9090 | TBP | TTGGTGGGTGAGCACAAGGCCTT |
| 9100 | TBP | CAGACTTACCTACTAAATTGTTG |
| 9110 | TBP | AACCAGGAAATAACTCTGGCTCA |
| 9120 | TBP | TGTAGATTAAACCAGGAAATAAC |
| 9130 | TBP | TGGGTTTGATCATTCTGTAGATT |
| 9140 | TBP | CTGCTCTGACTTTAGCACCTAAG |
| 9150 | TBP | CGTCGTCTTCCTGAATCCCTTTA |
| 9160 | E2F4 | TAGTGAGTGGCGGCCCTGGGACT |
| 9170 | E2F4 | CCAGAGTGCATGAGCTCGGAGCT |
| 9180 | E2F4 | TATCTACAACCTGGACGAGAGTG |
| 9190 | E2F4 | CCTGGACTTCTGCACTGCCAGGG |
| 9200 | E2F4 | CTGACAGCTCTTTGGGGAGTTCC |
| 9210 | G6PD | AGCTGGAGAAGCCCAAGCCCATC |
| 9220 | G6PD | TCACCCCACTGCTGCACCAGATT |
| 9230 | KIF11 | ATGAAGATAAATTGATAGCACAA |
| 9240 | KIF11 | ATAGCACAAAATCTAGAACTTAA |
| 9250 | KIF11 | GTTTGACTAAGCTTAATTGCTTT |
| 9260 | KIF11 | CTTTCTGGAACAGGATCTGAAAC |
| 9270 | KIF11 | ATACCCATCAACACTGGTAAGAA |
| 9280 | KIF11 | TTCATCAATTGGCGGGGTTCCAT |
| 9290 | KIF11 | GCGGGGTTCCATTTTTCCAGGTA |
| 9300 | KIF11 | TCCCGCCTTAAATCCACAGCATA |
| 9310 | KIF11 | ACACACTGGAGAGGTCTAAAGTG |
| 9320 | KIF11 | CCTCTGCGAGCCCAGATCAACCT |
| 9330 | KIF11 | AGTTCTAGATTTTGTGCTATCAA |
| 9340 | KIF11 | TTATGGTTTCATTAAGTTCTAGA |
| 9350 | KIF11 | AGCTTAGTCAAACCAATTTTTAT |
| 9360 | KIF11 | CTCTTTTAAAGTACCTGTTGGGA |
| 9370 | KIF11 | TATTTCTCTTTTAAAGTACCTGT |
| 9380 | KIF11 | ACAGCTCAGGCTGTTTCCTTTTC |
| 9390 | KIF11 | TCTCTTCTTTGTTGTTTTCTGAA |
| 9400 | KIF11 | ACCGGAATTGTCTCTTCTTTGTT |
| 9410 | KIF11 | ATGAACAATCCACACCAGCATCT |
| 9420 | KIF11 | AAGGTTGATCTGGGCTCGCAGAG |
| 9430 | KIF11 | CCAACCCCCAAGTGAATTAAAGG |

TABLE 22
Cas12e guide RNAs

| SEQ | | Target Domain |
|---|---|---|
| ID NO | Gene | Sequence (DNA) |
| 9440 | GAPDH | TCTTCTAGGTATGACAACGAA |
| 9450 | GAPDH | CCAGCTCTCATACCATGAGTC |
| 9460 | TBP | TGCCCGAAACGCCGAATATAA |
| 9470 | TBP | CTCTGACCATTGTAGCGGTTT |
| 9480 | TBP | GTTCTGGGAAAATGGTGTGCA |
| 9490 | TBP | GGGAAAATGGTGTGCACAGGA |
| 9500 | TBP | TTTCCCTAGTGAAGAACAGTC |
| 9510 | TBP | CTAGTGAAGAACAGTCCAGAC |
| 9520 | TBP | AGCTAAGTTCTTGGACTTCAA |
| 9530 | TBP | TGGACTTCAAGATTCAGAATA |
| 9540 | TBP | AGATTCAGAATATGGTGGGGA |
| 9550 | TBP | GAATATGGTGGGGAGCTGTGA |
| 9560 | TBP | TATAAGGTTAGAAGGCCTTGT |
| 9570 | TBP | TTCTAGTTATGAGCCAGAGTT |
| 9580 | TBP | AGTTATGAGCCAGAGTTATTT |
| 9590 | TBP | TGGTTTAATCTACAGAATGAT |
| 9600 | TBP | CCTTATTTTTGTTTCTGGAAA |
| 9610 | TBP | GGAAAAGTTGTATTAACAGGT |
| 9620 | TBP | TAGGTGCTAAAGTCAGAGCAG |
| 9630 | TBP | AAAGGGATTCAGGAAGACGAC |
| 9640 | TBP | GGCACGAAGTGCAATGGTCTT |
| 9650 | TBP | GCGTTTCGGGCACGAAGTGCA |
| 9660 | TBP | TGGCTCTCTTATCCTCATGAT |
| 9670 | TBP | CAGAACTGAAAATCAGTGCCG |
| 9680 | TBP | TACGGCTACCTCTTGGCTCCT |
| 9690 | TBP | TGCTGCCAGTCTGGACTGTTC |
| 9700 | TBP | GTACAACTCTAGCATATTTTC |
| 9710 | TBP | GAATCTTGAAGTCCAAGAACT |
| 9720 | TBP | CATCACAGCTCCCCACCATAT |
| 9730 | TBP | AACCTTATAGGAAACTTCACA |
| 9740 | TBP | GACTTACCTACTAAATTGTTG |
| 9750 | TBP | GTAGATTAAACCAGGAAATAA |
| 9760 | TBP | GGGTTTGATCATTCTGTAGAT |
| 9770 | TBP | AGAAACAAAAATAAGGAGAAC |
| 9780 | TBP | TGTTACAACTTACCTGTTAAT |
| 9790 | TBP | GCTCTGACTTTAGCACCTAAG |
| 9800 | TBP | TAAATTTCTGCTCTGACTTTA |
| 9810 | TBP | AATGCTTCATAAATTTCTGCT |
| 9820 | TBP | TGAATCCCTTTAGAATAGGGT |
| 9830 | E2F4 | CTCCCACTGGGCCCAACAACA |
| 9840 | E2F4 | GCCCTGCTGGACAGCAGCAGC |
| 9850 | E2F4 | TCCGGACCCAACCCTTCTACC |
| 9860 | E2F4 | ACCTCCTTTGAGCCCATCAAG |
| 9870 | E2F4 | TGTTTTTCAGTTTTGGAACTC |
| 9880 | E2F4 | GTTTTGGAACTCCCCAAAGAG |
| 9890 | E2F4 | CAGAGTGCATGAGCTCGGAGC |
| 9900 | E2F4 | TCTTTCTCCACCCCCGGGAGA |
| 9910 | E2F4 | CCACCCCCGGGAGACCACGAT |
| 9920 | E2F4 | GCACTGCCAGGGACAGCAGTG |
| 9930 | E2F4 | CTGGACTTCTGCACTGCCAGG |
| 9940 | E2F4 | GACAGCTCTTTGGGGAGTTCC |
| 9950 | E2F4 | GAGGACATCAACTCCTCCAGC |
| 9960 | E2F4 | AGGGCCACCCACCTTCTGAGG |
| 9970 | E2F4 | CTCTCGTCCAGGTTGTAGATA |
| 9980 | G6PD | CCCACTTGTAGGTGCCCTCAT |
| 9990 | G6PD | TCAGCTCGTCTGCCTCCGTGG |
| 10000 | G6PD | TCACCTGCCATAAATATAGGG |
| 10010 | G6PD | CCAGCTCAATCTGGTGCAGCA |
| 10020 | G6PD | CTGTAGGGCACCTTGTATCTG |
| 10030 | G6PD | TGGTCATCATCTTGGTGTACA |
| 10040 | G6PD | GGGCCTTGCCGCAGCGCAGGA |
| 10050 | G6PD | AGTATGAGGGCACCTACAAGT |
| 10060 | G6PD | CCCCACTGCTGCACCAGATTG |
| 10070 | G6PD | GCGGGAGCCAGATGCACTTCG |
| 10080 | G6PD | ACCCCGAGGAGTCGGAGCTGG |
| 10090 | G6PD | TCAACCCCGAGGAGTCGGAGC |
| 10100 | G6PD | ACCAGCAGTGCAAGCGCAACG |
| 10110 | G6PD | ATGATGTGGCCGGCGACATCT |
| 10120 | G6PD | TCCTGCGCTGCGGCAAGGCCC |
| 10130 | G6PD | GCCACGTAGGGGTGCCCTTCA |
| 10140 | KIF11 | GGAACAGGATCTGAAACTGGA |
| 10150 | KIF11 | GAAAACAACAAAGAAGAGACA |
| 10160 | KIF11 | TCTTTTAGGATGTGGATGTAG |
| 10170 | KIF11 | TTTAGGATGTGGATGTAGAAG |
| 10180 | KIF11 | GGGGCAGTATACTGAAGAACC |
| 10190 | KIF11 | TCAATTGGCGGGGTTCCATTT |
| 10200 | KIF11 | CGCCTTAAATCCACAGCATAA |
| 10210 | KIF11 | AGATTTTGTGCTATCAATTTA |
| 10220 | KIF11 | TTAAGTTCTAGATTTTGTGCT |
| 10230 | KIF11 | AGAAAGCAATTAAGCTTAGTC |
| 10240 | KIF11 | GATCCTGTTCCAGAAAGCAAT |
| 10250 | KIF11 | CTTTTAAAGTACCTGTTGGGA |
| 10260 | KIF11 | ATTTCTCTTTTAAAGTACCTG |
| 10270 | KIF11 | TCTGTGGTGTCGTACCTTTAA |
| 10280 | KIF11 | TACCAGTGTTGATGGGTATAA |
| 10290 | KIF11 | GTTCTTACCAGTGTTGATGGG |
| 10300 | KIF11 | CGTGGTTCAGTTCTTACCAGT |
| 10310 | KIF11 | GCTGATCAAGGAGATGTTCAC |
| 10320 | KIF11 | TTTTCAGCTGATCAAGGAGAT |
| 10330 | KIF11 | GAACAGTTTAGCATCATTAAC |
| 10340 | KIF11 | TTGTTGTTTTCTGAACAGTTT |
| 10350 | KIF11 | GTATACTGCCCCAGAACTGCC |
| 10360 | KIF11 | TCAGTATACTGCCCCAGAACT |
| 10370 | KIF11 | ATGTGATTTTTTATGCTGTGG |
| 10380 | KIF11 | TTGTCTTTTCCATGTGATTTT |
| 10390 | KIF11 | ACTTTAGACCTCTCCAGTGTG |
| 10400 | KIF11 | TCCACTTTAGACCTCTCCAGT |
| — | — | — |

TABLE 23
Cas-Phi guide RNAs

| SEQ | | |
|---|---|---|
| ID NO | Gene | Target Domain Sequence (DNA) |
| 10410 | GAPDH | TGCAGACCACAGTCCATGCCA |
| 10420 | GAPDH | GCAGACCACAGTCCATGCCAT |
| 10430 | GAPDH | CAGACCACAGTCCATGCCATC |
| 10440 | GAPDH | TCATCTTCTAGGTATGACAAC |
| 10450 | GAPDH | CATCTTCTAGGTATGACAACG |
| 10460 | GAPDH | ATCTTCTAGGTATGACAACGA |
| 10470 | GAPDH | TAGGTATGACAACGAATTTGG |
| 10480 | GAPDH | CCCAGCTCTCATACCATGAGT |
| 10490 | TBP | TATCCACAGTGAATCTTGGTT |
| 10500 | TBP | GTTGTAAACTTGACCTAAAGA |
| 10510 | TBP | TAAACTTGACCTAAAGACCAT |
| 10520 | TBP | ACCTAAAGACCATTGCACTTC |
| 10530 | TBP | CACTTCGTGCCCGAAACGCCG |
| 10540 | TBP | GTGCCCGAAACGCCGAATATA |
| 10550 | TBP | TCTCTGACCATTGTAGCGGTT |
| 10560 | TBP | TAGCGGTTTGCTGCGGTAATC |
| 10570 | TBP | GCTGCGGTAATCATGAGGATA |
| 10580 | TBP | CTGCGGTAATCATGAGGATAA |
| 10590 | TBP | TCAGTTCTGGGAAAATGGTGT |
| 10600 | TBP | CAGTTCTGGGAAAATGGTGTG |
| 10610 | TBP | AGTTCTGGGAAAATGGTGTGC |
| 10620 | TBP | TGGGAAAATGGTGTGCACAGG |
| 10630 | TBP | TTTCCTTTCCCTAGTGAAGAA |
| 10640 | TBP | TTCCTTTCCCTAGTGAAGAAC |
| 10650 | TBP | TCCTTTCCCTAGTGAAGAACA |
| 10660 | TBP | CCTTTCCCTAGTGAAGAACAG |
| 10670 | TBP | CTTTCCCTAGTGAAGAACAGT |
| 10680 | TBP | CCCTAGTGAAGAACAGTCCAG |
| 10690 | TBP | CCTAGTGAAGAACAGTCCAGA |
| 10700 | TBP | TACAGAAGTTGGGTTTTCCAG |
| 10710 | TBP | GGTTTTCCAGCTAAGTTCTTG |
| 10720 | TBP | TCCAGCTAAGTTCTTGGACTT |
| 10730 | TBP | CCAGCTAAGTTCTTGGACTTC |
| 10740 | TBP | CAGCTAAGTTCTTGGACTTCA |
| 10750 | TBP | TTGGACTTCAAGATTCAGAAT |
| 10760 | TBP | GACTTCAAGATTCAGAATATG |
| 10770 | TBP | AAGATTCAGAATATGGTGGGG |
| 10780 | TBP | AGAATATGGTGGGGAGCTGTG |
| 10790 | TBP | CCTATAAGGTTAGAAGGCCTT |
| 10800 | TBP | CTATAAGGTTAGAAGGCCTTG |
| 10810 | TBP | TGCTCACCCACCAACAATTTA |
| 10820 | TBP | TTGCAATTTTCCTTCTAGTTA |
| 10830 | TBP | TGCAATTTTCCTTCTAGTTAT |
| 10840 | TBP | GCAATTTTCCTTCTAGTTATG |
| 10850 | TBP | CAATTTTCCTTCTAGTTATGA |
| 10860 | TBP | TCCTTCTAGTTATGAGCCAGA |
| 10870 | TBP | CCTTCTAGTTATGAGCCAGAG |
| 10880 | TBP | CTTCTAGTTATGAGCCAGAGT |
| 10890 | TBP | TAGTTATGAGCCAGAGTTATT |
| 10900 | TBP | TGAGCCAGAGTTATTTCCTGG |
| 10910 | TBP | CCTGGTTTAATCTACAGAATG |
| 10920 | TBP | CTGGTTTAATCTACAGAATGA |
| 10930 | TBP | AATCTACAGAATGATCAAACC |
| 10940 | TBP | ATCTACAGAATGATCAAACCC |
| 10950 | TBP | TTCTCCTTATTTTTGTTTCTG |
| 10960 | TBP | TCCTTATTTTTGTTTCTGGAA |
| 10970 | TBP | TTTTTGTTTCTGGAAAAGTTG |
| 10980 | TBP | TTGTTTCTGGAAAAGTTGTAT |
| 10990 | TBP | TGTTTCTGGAAAAGTTGTATT |
| 11000 | TBP | GTTTCTGGAAAAGTTGTATTA |
| 11010 | TBP | TTTCTGGAAAAGTTGTATTAA |
| 11020 | TBP | CTGGAAAAGTTGTATTAACAG |
| 11030 | TBP | TGGAAAAGTTGTATTAACAGG |
| 11040 | TBP | TCTTCTTAGGTGCTAAAGTCA |
| 11050 | TBP | TTAGGTGCTAAAGTCAGAGCA |
| 11060 | TBP | GGTGCTAAAGTCAGAGCAGAA |
| 11070 | TBP | TAAAGGGATTCAGGAAGACGA |
| 11080 | TBP | GGTCAAGTTTACAACCAAGAT |
| 11090 | TBP | AGGTCAAGTTTACAACCAAGA |
| 11100 | TBP | GGGCACGAAGTGCAATGGTCT |
| 11110 | TBP | CGGGCACGAAGTGCAATGGTC |
| 11120 | TBP | GGCGTTTCGGGCACGAAGTGC |
| 11130 | TBP | TATTCGGCGTTTCGGGCACGA |
| 11140 | TBP | GGATTATATTCGGCGTTTCGG |
| 11150 | TBP | AAATAGATCTAACCTTGGGAT |
| 11160 | TBP | TCCTCATGATTACCGCAGCAA |
| 11170 | TBP | GTGGCTCTCTTATCCTCATGA |
| 11180 | TBP | CCAGAACTGAAAATCAGTGCC |
| 11190 | TBP | CCCAGAACTGAAAATCAGTGC |
| 11200 | TBP | TCCCAGAACTGAAAATCAGTG |
| 11210 | TBP | GCTCCTGTGCACACCATTTTC |
| 11220 | TBP | CGGCTACCTCTTGGCTCCTGT |
| 11230 | TBP | TTACGGCTACCTCTTGGCTCC |
| 11240 | TBP | CTTACGGCTACCTCTTGGCTC |
| 11250 | TBP | CTGCCAGTCTGGACTGTTCTT |
| 11260 | TBP | TTGCTGCCAGTCTGGACTGTT |
| 11270 | TBP | CTTGCTGCCAGTCTGGACTGT |
| 11280 | TBP | TCTTGCTGCCAGTCTGGACTG |
| 11290 | TBP | TGTACAACTCTAGCATATTTT |
| 11300 | TBP | GCTGGAAAACCCAACTTCTGT |
| 11310 | TBP | AAGTCCAAGAACTTAGCTGGA |
| 11320 | TBP | TGAATCTTGAAGTCCAAGAAC |
| 11330 | TBP | ACATCACAGCTCCCCACCATA |
| 11340 | TBP | TAACCTTATAGGAAACTTCAC |
| 11350 | TBP | GTGGGTGAGCACAAGGCCTTC |
| 11360 | TBP | TTGGTGGGTGAGCACAAGGCC |
| 11370 | TBP | CCTACTAAATTGTTGGTGGGT |
| 11380 | TBP | AGACTTACCTACTAAATTGTT |
| 11390 | TBP | CAGACTTACCTACTAAATTGT |
| 11400 | TBP | AACCAGGAAATAACTCTGGCT |
| 11410 | TBP | TGTAGATTAAACCAGGAAATA |
| 11420 | TBP | ATCATTCTGTAGATTAAACCA |
| 11430 | TBP | GATCATTCTGTAGATTAAACC |
| 11440 | TBP | TGGGTTTGATCATTCTGTAGA |
| 11450 | TBP | CAGAAACAAAAATAAGGAGAA |
| 11460 | TBP | CCAGAAACAAAAATAAGGAGA |
| 11470 | TBP | TCCAGAAACAAAAATAAGGAG |
| 11480 | TBP | ATACAACTTTTCCAGAAACAA |
| 11490 | TBP | CCTGTTAATACAACTTTTCCA |
| 11500 | TBP | CAACTTACCTGTTAATACAAC |
| 11510 | TBP | CTGTTACAACTTACCTGTTAA |
| 11520 | TBP | TGCTCTGACTTTAGCACCTAA |
| 11530 | TBP | CTGCTCTGACTTTAGCACCTA |
| 11540 | TBP | ATAAATTTCTGCTCTGACTTT |
| 11550 | TBP | AAATGCTTCATAAATTTCTGC |
| 11560 | TBP | CAAATGCTTCATAAATTTCTG |
| 11570 | TBP | TCAAATGCTTCATAAATTTCT |
| 11580 | TBP | CTGAATCCCTTTAGAATAGGG |
| 11590 | TBP | CGTCGTCTTCCTGAATCCCTT |
| 11600 | E2F4 | GGGGGCTATCATTGTAGTGAG |
| 11610 | E2F4 | GGGGCTATCATTGTAGTGAGT |
| 11620 | E2F4 | TAGTGAGTGGCGGCCCTGGGA |
| 11630 | E2F4 | ACTCCCACTGGGCCCAACAAC |
| 11640 | E2F4 | TGCCCTGCTGGACAGCAGCAG |
| 11650 | E2F4 | GTCCGGACCCAACCCTTCTAC |
| 11660 | E2F4 | TACCTCCTTTGAGCCCATCAA |
| 11670 | E2F4 | GAGCCCATCAAGGCAGACCCC |
| 11680 | E2F4 | AGCCCATCAAGGCAGACCCCA |
| 11690 | E2F4 | CTTGTTTTTCAGTTTTGGAAC |
| 11700 | E2F4 | TTTTTCAGTTTTGGAACTCCC |
| 11710 | E2F4 | TTCAGTTTTGGAACTCCCCAA |
| 11720 | E2F4 | TCAGTTTTGGAACTCCCCAAA |
| 11730 | E2F4 | CAGTTTTGGAACTCCCCAAAG |
| 11740 | E2F4 | AGTTTTGGAACTCCCCAAAGA |
| 11750 | E2F4 | TGGAACTCCCCAAAGAGCTGT |
| 11760 | E2F4 | GGAACTCCCCAAAGAGCTGTC |
| 11770 | E2F4 | CCAGAGTGCATGAGCTCGGAG |
| 11780 | E2F4 | GCCCCTCTGCTTCGTCTTTCT |
| 11790 | E2F4 | CCCCTCTGCTTCGTCTTTCTC |
| 11800 | E2F4 | GTCTTTCTCCACCCCCGGGAG |
| 11810 | E2F4 | CTCCACCCCCGGGAGACCACG |
| 11820 | E2F4 | TCCACCCCCGGGAGACCACGA |
| 11830 | E2F4 | TATCTACAACCTGGACGAGAG |
| 11840 | E2F4 | GATGTGCCTGTTCTCAACCTC |
| 11850 | E2F4 | ATGTGCCTGTTCTCAACCTCT |
| 11860 | E2F4 | TGCACTGCCAGGGACAGCAGT |
| 11870 | E2F4 | CCTGGACTTCTGCACTGCCAG |
| 11880 | E2F4 | CTATCAGTCCCAGGGCCGCCA |
| 11890 | E2F4 | GGCCCAGTGGGAGTGAACTGA |
| 11900 | E2F4 | TTGGGCCCAGTGGGAGTGAAC |
| 11910 | E2F4 | GGTCCGGACGAACTGCTGCTG |
| 11920 | E2F4 | ATGGGCTCAAAGGAGGTAGAA |
| 11930 | E2F4 | TGACAGCTCTTTGGGGAGTTC |
| 11940 | E2F4 | CTGACAGCTCTTTGGGGAGTT |
| 11950 | E2F4 | TGAGGACATCAACTCCTCCAG |
| 11960 | E2F4 | CAGGGCCACCCACCTTCTGAG |
| 11970 | E2F4 | TAGATATAATCGTGGTCTCCC |
| 11980 | E2F4 | ACTCTCGTCCAGGTTGTAGAT |
| 11990 | G6PD | TGGGGGTTCACCCACTTGTAG |
| 12000 | G6PD | ACCCACTTGTAGGTGCCCTCA |
| 12010 | G6PD | TAGGTGCCCTCATACTGGAAA |
| 12020 | G6PD | ATCAGCTCGTCTGCCTCCGTG |
| 12030 | G6PD | CCTCACCTGCCATAAATATAG |
| 12040 | G6PD | CTCACCTGCCATAAATATAGG |
| 12050 | G6PD | GGCTTCTCCAGCTCAATCTGG |
| 12060 | G6PD | TCCAGCTCAATCTGGTGCAGC |
| 12070 | G6PD | TCTGTAGGGCACCTTGTATCT |
| 12080 | G6PD | TATCTGTTGCCGTAGGTCAGG |
| 12090 | G6PD | CCGTAGGTCAGGTCCAGCTCC |
| 12100 | G6PD | AAGAACATGCCCGGCTTCTTG |
| 12110 | G6PD | TTGGTCATCATCTTGGTGTAC |
| 12120 | G6PD | GTCATCATCTTGGTGTACACG |
| 12130 | G6PD | GTGTACACGGCCTCGTTGGGC |
| 12140 | G6PD | GGCTGCACGCGGATCACCAGC |
| 12150 | G6PD | CGCTTGCACTGCTGGTGGAAG |
| 12160 | G6PD | CACTGCTGGTGGAAGATGTCG |
| 12170 | G6PD | CGCTCGTTCAGGGCCTTGCCG |
| 12180 | G6PD | AGGGCCTTGCCGCAGCGCAGG |
| 12190 | G6PD | CCGCAGCGCAGGATGAAGGGC |
| 12200 | G6PD | CAGTATGAGGGCACCTACAAG |
| 12210 | G6PD | CCAGTATGAGGGCACCTACAA |
| 12220 | G6PD | AGCTGGAGAAGCCCAAGCCCA |
| 12230 | G6PD | ACCCCACTGCTGCACCAGATT |
| 12240 | G6PD | CACCCCACTGCTGCACCAGAT |
| 12250 | G6PD | TCACCCCACTGCTGCACCAGA |
| 12260 | G6PD | TGCGGGAGCCAGATGCACTTC |
| 12270 | G6PD | AACCCCGAGGAGTCGGAGCTG |
| 12280 | G6PD | TTCAACCCCGAGGAGTCGGAG |
| 12290 | G6PD | CACCAGCAGTGCAAGCGCAAC |
| 12300 | G6PD | CATGATGTGGCCGGCGACATC |
| 12310 | G6PD | ATCCTGCGCTGCGGCAAGGCC |
| 12320 | G6PD | CGCCACGTAGGGGTGCCCTTC |
| 12330 | G6PD | CCGCCACGTAGGGGTGCCCTT |
| 12340 | KIF11 | ATGAAGATAAATTGATAGCAC |
| 12350 | KIF11 | ATAGCACAAAATCTAGAACTT |
| 12360 | KIF11 | ATGAAACCATAAAAATTGGTT |
| 12370 | KIF11 | GTTTGACTAAGCTTAATTGCT |
| 12380 | KIF11 | GACTAAGCTTAATTGCTTTCT |
| 12390 | KIF11 | ACTAAGCTTAATTGCTTTCTG |
| 12400 | KIF11 | ATTGCTTTCTGGAACAGGATC |
| 12410 | KIF11 | CTTTCTGGAACAGGATCTGAA |
| 12420 | KIF11 | CTGGAACAGGATCTGAAACTG |
| 12430 | KIF11 | TGGAACAGGATCTGAAACTGG |
| 12440 | KIF11 | TCTAATGTCCGTTAAAGGTAC |
| 12450 | KIF11 | AAGGTACGACACCACAGAGGA |
| 12460 | KIF11 | TTTATACCCATCAACACTGGT |
| 12470 | KIF11 | ATACCCATCAACACTGGTAAG |
| 12480 | KIF11 | TACCCATCAACACTGGTAAGA |
| 12490 | KIF11 | ATCAGCTGAAAAGGAAACAGC |
| 12500 | KIF11 | ATGATGCTAAACTGTTCAGAA |
| 12510 | KIF11 | AGAAAACAACAAAGAAGAGAC |
| 12520 | KIF11 | CTTCTTTTAGGATGTGGATGT |
| 12530 | KIF11 | TTCTTTTAGGATGTGGATGTA |
| 12540 | KIF11 | TTTTAGGATGTGGATGTAGAA |
| 12550 | KIF11 | TAGGATGTGGATGTAGAAGAG |
| 12560 | KIF11 | AGGATGTGGATGTAGAAGAGG |
| 12570 | KIF11 | GGATGTGGATGTAGAAGAGGC |
| 12580 | KIF11 | TGGGGCAGTATACTGAAGAAC |
| 12590 | KIF11 | TTCATCAATTGGCGGGGTTCC |
| 12600 | KIF11 | ATCAATTGGCGGGGTTCCATT |
| 12610 | KIF11 | GCGGGGTTCCATTTTTCCAGG |
| 12620 | KIF11 | TCCCGCCTTAAATCCACAGCA |
| 12630 | KIF11 | CCCGCCTTAAATCCACAGCAT |
| 12640 | KIF11 | CCGCCTTAAATCCACAGCATA |
| 12650 | KIF11 | AATCCACAGCATAAAAAATCA |
| 12660 | KIF11 | ACACACTGGAGAGGTCTAAAG |
| 12670 | KIF11 | GTTACAAAGAGCAGATTACCT |
| 12680 | KIF11 | CAAAGAGCAGATTACCTCTGC |
| 12690 | KIF11 | CCTCTGCGAGCCCAGATCAAC |
| 12700 | KIF11 | TAGATTTTGTGCTATCAATTT |
| 12710 | KIF11 | AGTTCTAGATTTTGTGCTATC |
| 12720 | KIF11 | ATTAAGTTCTAGATTTTGTGC |
| 12730 | KIF11 | CATTAAGTTCTAGATTTTGTG |
| 12740 | KIF11 | TGGTTTCATTAAGTTCTAGAT |
| 12750 | KIF11 | ATGGTTTCATTAAGTTCTAGA |
| 12760 | KIF11 | TATGGTTTCATTAAGTTCTAG |
| 12770 | KIF11 | TTATGGTTTCATTAAGTTCTA |
| 12780 | KIF11 | GTCAAACCAATTTTTATGGTT |
| 12790 | KIF11 | AGCTTAGTCAAACCAATTTTT |
| 12800 | KIF11 | CAGAAAGCAATTAAGCTTAGT |
| 12810 | KIF11 | AGATCCTGTTCCAGAAAGCAA |
| 12820 | KIF11 | CAGATCCTGTTCCAGAAAGCA |
| 12830 | KIF11 | GGATATCCAGTTTCAGATCCT |
| 12840 | KIF11 | AAGTACCTGTTGGGATATCCA |
| 12850 | KIF11 | AAAGTACCTGTTGGGATATCC |
| 12860 | KIF11 | TAAAGTACCTGTTGGGATATC |
| 12870 | KIF11 | TCTTTTAAAGTACCTGTTGGG |
| 12880 | KIF11 | CTCTTTTAAAGTACCTGTTGG |
| 12890 | KIF11 | TATTTCTCTTTTAAAGTACCT |
| 12900 | KIF11 | CTCTGTGGTGTCGTACCTTTA |
| 12910 | KIF11 | CCTCTGTGGTGTCGTACCTTT |
| 12920 | KIF11 | TCCTCTGTGGTGTCGTACCTT |
| 12930 | KIF11 | ATGGGTATAAATAACTTTTCC |
| 12940 | KIF11 | CCAGTGTTGATGGGTATAAAT |
| 12950 | KIF11 | TTACCAGTGTTGATGGGTATA |
| 12960 | KIF11 | AGTTCTTACCAGTGTTGATGG |
| 12970 | KIF11 | ACGTGGTTCAGTTCTTACCAG |
| 12980 | KIF11 | AGCTGATCAAGGAGATGTTCA |
| 12990 | KIF11 | CAGCTGATCAAGGAGATGTTC |
| 13000 | KIF11 | TCAGCTGATCAAGGAGATGTT |
| 13010 | KIF11 | CTTTTCAGCTGATCAAGGAGA |
| 13020 | KIF11 | CCTTTTCAGCTGATCAAGGAG |
| 13030 | KIF11 | ACAGCTCAGGCTGTTTCCTTT |
| 13040 | KIF11 | GCATCATTAACAGCTCAGGCT |
| 13050 | KIF11 | AGCATCATTAACAGCTCAGGC |
| 13060 | KIF11 | TGAACAGTTTAGCATCATTAA |
| 13070 | KIF11 | CTGAACAGTTTAGCATCATTA |
| 13080 | KIF11 | TCTGAACAGTTTAGCATCATT |
| 13090 | KIF11 | TTTTCTGAACAGTTTAGCATC |
| 13100 | KIF11 | TTGTTTTCTGAACAGTTTAGC |
| 13110 | KIF11 | TTTGTTGTTTTCTGAACAGTT |
| 13120 | KIF11 | TCTCTTCTTTGTTGTTTTCTG |
| 13130 | KIF11 | CCGGAATTGTCTCTTCTTTGT |
| 13140 | KIF11 | ACCGGAATTGTCTCTTCTTTG |
| 13150 | KIF11 | AATTTACCGGAATTGTCTCTT |
| 13160 | KIF11 | AAATTTACCGGAATTGTCTCT |
| 13170 | KIF11 | AGTATACTGCCCCAGAACTGC |
| 13180 | KIF11 | TTCAGTATACTGCCCCAGAAC |
| 13190 | KIF11 | GAGGTTCTTCAGTATACTGCC |
| 13200 | KIF11 | ACTTAGAGGTTCTTCAGTATA |
| 13210 | KIF11 | ATGAACAATCCACACCAGCAT |
| 13220 | KIF11 | TCTGATATGACATACCTGGAA |
| 13230 | KIF11 | CATGTGATTTTTTATGCTGTG |
| 13240 | KIF11 | CCATGTGATTTTTTATGCTGT |
| 13250 | KIF11 | TCCATGTGATTTTTTATGCTG |
| 13260 | KIF11 | TCTTTTCCATGTGATTTTTTA |
| 13270 | KIF11 | GTCTTTTCCATGTGATTTTTT |
| 13280 | KIF11 | TTTGTCTTTTCCATGTGATTT |
| 13290 | KIF11 | CTTTGTCTTTTCCATGTGATT |
| 13300 | KIF11 | TCTTTGTCTTTTCCATGTGAT |
| 13310 | KIF11 | ATGCCTCTGTTTTCTTTGTCT |
| 13320 | KIF11 | GACCTCTCCAGTGTGTTAATG |
| 13330 | KIF11 | AGACCTCTCCAGTGTGTTAAT |
| 13340 | KIF11 | CACTTTAGACCTCTCCAGTGT |
| 13350 | KIF11 | TTCCACTTTAGACCTCTCCAG |
| 13360 | KIF11 | CTTCCACTTTAGACCTCTCCA |
| 13370 | KIF11 | TAACCAAGTGCTCTGTAGTTT |
| 13380 | KIF11 | GTAACCAAGTGCTCTGTAGTT |
| 13390 | KIF11 | ATCTGGGCTCGCAGAGGTAAT |
| 13400 | KIF11 | AAGGTTGATCTGGGCTCGCAG |
| 13410 | KIF11 | CCAACCCCCAAGTGAATTAAA |
| — | — | — |

TABLE 24
Mad7 guide RNAs

| SEQ | | Target Domain Sequence |
|---|---|---|
| ID NO | Gene | (DNA) |
| 13420 | GAPDH | TGCAGACCACAGTCCATGCCA |
| 13430 | GAPDH | GCAGACCACAGTCCATGCCAT |
| 13440 | GAPDH | CAGACCACAGTCCATGCCATC |
| 13450 | GAPDH | TCATCTTCTAGGTATGACAAC |
| 13460 | GAPDH | CATCTTCTAGGTATGACAACG |
| 13470 | GAPDH | ATCTTCTAGGTATGACAACGA |
| 13480 | GAPDH | TAGGTATGACAACGAATTTGG |
| 13490 | GAPDH | CCCAGCTCTCATACCATGAGT |
| 13500 | TBP | TATCCACAGTGAATCTTGGTT |
| 13510 | TBP | GTTGTAAACTTGACCTAAAGA |
| 13520 | TBP | TAAACTTGACCTAAAGACCAT |
| 13530 | TBP | ACCTAAAGACCATTGCACTTC |
| 13540 | TBP | CACTTCGTGCCCGAAACGCCG |
| 13550 | TBP | GTGCCCGAAACGCCGAATATA |
| 13560 | TBP | TCTCTGACCATTGTAGCGGTT |
| 13570 | TBP | TAGCGGTTTGCTGCGGTAATC |
| 13580 | TBP | GCTGCGGTAATCATGAGGATA |
| 13590 | TBP | CTGCGGTAATCATGAGGATAA |
| 13600 | TBP | TCAGTTCTGGGAAAATGGTGT |
| 13610 | TBP | CAGTTCTGGGAAAATGGTGTG |
| 13620 | TBP | AGTTCTGGGAAAATGGTGTGC |
| 13630 | TBP | TGGGAAAATGGTGTGCACAGG |
| 13640 | TBP | TTTCCTTTCCCTAGTGAAGAA |
| 13650 | TBP | TTCCTTTCCCTAGTGAAGAAC |
| 13660 | TBP | TCCTTTCCCTAGTGAAGAACA |
| 13670 | TBP | CCTTTCCCTAGTGAAGAACAG |
| 13680 | TBP | CTTTCCCTAGTGAAGAACAGT |
| 13690 | TBP | CCCTAGTGAAGAACAGTCCAG |
| 13700 | TBP | CCTAGTGAAGAACAGTCCAGA |
| 13710 | TBP | TACAGAAGTTGGGTTTTCCAG |
| 13720 | TBP | GGTTTTCCAGCTAAGTTCTTG |
| 13730 | TBP | TCCAGCTAAGTTCTTGGACTT |
| 13740 | TBP | CCAGCTAAGTTCTTGGACTTC |
| 13750 | TBP | CAGCTAAGTTCTTGGACTTCA |
| 13760 | TBP | TTGGACTTCAAGATTCAGAAT |
| 13770 | TBP | GACTTCAAGATTCAGAATATG |
| 13780 | TBP | AAGATTCAGAATATGGTGGGG |
| 13790 | TBP | AGAATATGGTGGGGAGCTGTG |
| 13800 | TBP | CCTATAAGGTTAGAAGGCCTT |
| 13810 | TBP | CTATAAGGTTAGAAGGCCTTG |
| 13820 | TBP | TGCTCACCCACCAACAATTTA |
| 13830 | TBP | TTGCAATTTTCCTTCTAGTTA |
| 13840 | TBP | TGCAATTTTCCTTCTAGTTAT |
| 13850 | TBP | GCAATTTTCCTTCTAGTTATG |
| 13860 | TBP | CAATTTTCCTTCTAGTTATGA |
| 13870 | TBP | TCCTTCTAGTTATGAGCCAGA |
| 13880 | TBP | CCTTCTAGTTATGAGCCAGAG |
| 13890 | TBP | CTTCTAGTTATGAGCCAGAGT |
| 13900 | TBP | TAGTTATGAGCCAGAGTTATT |
| 13910 | TBP | TGAGCCAGAGTTATTTCCTGG |
| 13920 | TBP | CCTGGTTTAATCTACAGAATG |
| 13930 | TBP | CTGGTTTAATCTACAGAATGA |
| 13940 | TBP | AATCTACAGAATGATCAAACC |
| 13950 | TBP | ATCTACAGAATGATCAAACCC |
| 13960 | TBP | TTCTCCTTATTTTTGTTTCTG |
| 13970 | TBP | TCCTTATTTTTGTTTCTGGAA |
| 13980 | TBP | TTTTTGTTTCTGGAAAAGTTG |
| 13990 | TBP | TTGTTTCTGGAAAAGTTGTAT |
| 14000 | TBP | TGTTTCTGGAAAAGTTGTATT |
| 14010 | TBP | GTTTCTGGAAAAGTTGTATTA |
| 14020 | TBP | TTTCTGGAAAAGTTGTATTAA |
| 14030 | TBP | CTGGAAAAGTTGTATTAACAG |
| 14040 | TBP | TGGAAAAGTTGTATTAACAGG |
| 14050 | TBP | TCTTCTTAGGTGCTAAAGTCA |
| 14060 | TBP | TTAGGTGCTAAAGTCAGAGCA |
| 14070 | TBP | GGTGCTAAAGTCAGAGCAGAA |
| 14080 | TBP | TAAAGGGATTCAGGAAGACGA |
| 14090 | TBP | GGTCAAGTTTACAACCAAGAT |
| 14100 | TBP | AGGTCAAGTTTACAACCAAGA |
| 14110 | TBP | GGGCACGAAGTGCAATGGTCT |
| 14120 | TBP | CGGGCACGAAGTGCAATGGTC |
| 14130 | TBP | GGCGTTTCGGGCACGAAGTGC |
| 14140 | TBP | TATTCGGCGTTTCGGGCACGA |
| 14150 | TBP | GGATTATATTCGGCGTTTCGG |
| 14160 | TBP | AAATAGATCTAACCTTGGGAT |
| 14170 | TBP | TCCTCATGATTACCGCAGCAA |
| 14180 | TBP | GTGGCTCTCTTATCCTCATGA |
| 14190 | TBP | CCAGAACTGAAAATCAGTGCC |
| 14200 | TBP | CCCAGAACTGAAAATCAGTGC |
| 14210 | TBP | TCCCAGAACTGAAAATCAGTG |
| 14220 | TBP | GCTCCTGTGCACACCATTTTC |
| 14230 | TBP | CGGCTACCTCTTGGCTCCTGT |
| 14240 | TBP | TTACGGCTACCTCTTGGCTCC |
| 14250 | TBP | CTTACGGCTACCTCTTGGCTC |
| 14260 | TBP | CTGCCAGTCTGGACTGTTCTT |
| 14270 | TBP | TTGCTGCCAGTCTGGACTGTT |
| 14280 | TBP | CTTGCTGCCAGTCTGGACTGT |
| 14290 | TBP | TCTTGCTGCCAGTCTGGACTG |
| 14300 | TBP | TGTACAACTCTAGCATATTTT |
| 14310 | TBP | GCTGGAAAACCCAACTTCTGT |
| 14320 | TBP | AAGTCCAAGAACTTAGCTGGA |
| 14330 | TBP | TGAATCTTGAAGTCCAAGAAC |
| 14340 | TBP | ACATCACAGCTCCCCACCATA |
| 14350 | TBP | TAACCTTATAGGAAACTTCAC |
| 14360 | TBP | GTGGGTGAGCACAAGGCCTTC |
| 14370 | TBP | TTGGTGGGTGAGCACAAGGCC |
| 14380 | TBP | CCTACTAAATTGTTGGTGGGT |
| 14390 | TBP | AGACTTACCTACTAAATTGTT |
| 14400 | TBP | CAGACTTACCTACTAAATTGT |
| 14410 | TBP | AACCAGGAAATAACTCTGGCT |
| 14420 | TBP | TGTAGATTAAACCAGGAAATA |
| 14430 | TBP | ATCATTCTGTAGATTAAACCA |
| 14440 | TBP | GATCATTCTGTAGATTAAACC |
| 14450 | TBP | TGGGTTTGATCATTCTGTAGA |
| 14460 | TBP | CAGAAACAAAAATAAGGAGAA |
| 14470 | TBP | CCAGAAACAAAAATAAGGAGA |
| 14480 | TBP | TCCAGAAACAAAAATAAGGAG |
| 14490 | TBP | ATACAACTTTTCCAGAAACAA |
| 14500 | TBP | CCTGTTAATACAACTTTTCCA |
| 14510 | TBP | CAACTTACCTGTTAATACAAC |
| 14520 | TBP | CTGTTACAACTTACCTGTTAA |
| 14530 | TBP | ATAAATTTCTGCTCTGACTTT |
| 14540 | TBP | AAATGCTTCATAAATTTCTGC |
| 14550 | TBP | CAAATGCTTCATAAATTTCTG |
| 14560 | TBP | TCAAATGCTTCATAAATTTCT |
| 14570 | TBP | CTGAATCCCTTTAGAATAGGG |
| 14580 | TBP | CGTCGTCTTCCTGAATCCCTT |
| 14590 | E2F4 | GGGGGCTATCATTGTAGTGAG |
| 14600 | E2F4 | GGGGCTATCATTGTAGTGAGT |
| 14610 | E2F4 | TAGTGAGTGGCGGCCCTGGGA |
| 14620 | E2F4 | ACTCCCACTGGGCCCAACAAC |
| 14630 | E2F4 | TGCCCTGCTGGACAGCAGCAG |
| 14640 | E2F4 | GTCCGGACCCAACCCTTCTAC |
| 14650 | E2F4 | TACCTCCTTTGAGCCCATCAA |
| 14660 | E2F4 | GAGCCCATCAAGGCAGACCCC |
| 14670 | E2F4 | AGCCCATCAAGGCAGACCCCA |
| 14680 | E2F4 | CTTGTTTTTCAGTTTTGGAAC |
| 14690 | E2F4 | TTTTTCAGTTTTGGAACTCCC |
| 14700 | E2F4 | TTCAGTTTTGGAACTCCCCAA |
| 14710 | E2F4 | TCAGTTTTGGAACTCCCCAAA |
| 14720 | E2F4 | CAGTTTTGGAACTCCCCAAAG |
| 14730 | E2F4 | AGTTTTGGAACTCCCCAAAGA |
| 14740 | E2F4 | TGGAACTCCCCAAAGAGCTGT |
| 14750 | E2F4 | GGAACTCCCCAAAGAGCTGTC |
| 14760 | E2F4 | CCAGAGTGCATGAGCTCGGAG |
| 14770 | E2F4 | GCCCCTCTGCTTCGTCTTTCT |
| 14780 | E2F4 | CCCCTCTGCTTCGTCTTTCTC |
| 14790 | E2F4 | GTCTTTCTCCACCCCCGGGAG |
| 14800 | E2F4 | CTCCACCCCCGGGAGACCACG |
| 14810 | E2F4 | TCCACCCCCGGGAGACCACGA |
| 14820 | E2F4 | TATCTACAACCTGGACGAGAG |
| 14830 | E2F4 | GATGTGCCTGTTCTCAACCTC |
| 14840 | E2F4 | ATGTGCCTGTTCTCAACCTCT |
| 14850 | E2F4 | TGCACTGCCAGGGACAGCAGT |
| 14860 | E2F4 | CCTGGACTTCTGCACTGCCAG |
| 14870 | E2F4 | CTATCAGTCCCAGGGCCGCCA |
| 14880 | E2F4 | GGCCCAGTGGGAGTGAACTGA |
| 14890 | E2F4 | TTGGGCCCAGTGGGAGTGAAC |
| 14900 | E2F4 | GGTCCGGACGAACTGCTGCTG |
| 14910 | E2F4 | ATGGGCTCAAAGGAGGTAGAA |
| 14920 | E2F4 | TGACAGCTCTTTGGGGAGTTC |
| 14930 | E2F4 | CTGACAGCTCTTTGGGGAGTT |
| 14940 | E2F4 | TGAGGACATCAACTCCTCCAG |
| 14950 | E2F4 | CAGGGCCACCCACCTTCTGAG |
| 14960 | E2F4 | TAGATATAATCGTGGTCTCCC |
| 14970 | E2F4 | ACTCTCGTCCAGGTTGTAGAT |
| 14980 | G6PD | TGGGGGTTCACCCACTTGTAG |
| 14990 | G6PD | ACCCACTTGTAGGTGCCCTCA |
| 15000 | G6PD | TAGGTGCCCTCATACTGGAAA |
| 15010 | G6PD | ATCAGCTCGTCTGCCTCCGTG |
| 15020 | G6PD | CCTCACCTGCCATAAATATAG |
| 15030 | G6PD | CTCACCTGCCATAAATATAGG |
| 15040 | G6PD | GGCTTCTCCAGCTCAATCTGG |
| 15050 | G6PD | TCCAGCTCAATCTGGTGCAGC |
| 15060 | G6PD | TCTGTAGGGCACCTTGTATCT |
| 15070 | G6PD | TATCTGTTGCCGTAGGTCAGG |
| 15080 | G6PD | CCGTAGGTCAGGTCCAGCTCC |
| 15090 | G6PD | AAGAACATGCCCGGCTTCTTG |
| 15100 | G6PD | TTGGTCATCATCTTGGTGTAC |
| 15110 | G6PD | GTCATCATCTTGGTGTACACG |
| 15120 | G6PD | GTGTACACGGCCTCGTTGGGC |
| 15130 | G6PD | GGCTGCACGCGGATCACCAGC |
| 15140 | G6PD | CGCTTGCACTGCTGGTGGAAG |
| 15150 | G6PD | CACTGCTGGTGGAAGATGTCG |
| 15160 | G6PD | CGCTCGTTCAGGGCCTTGCCG |
| 15170 | G6PD | AGGGCCTTGCCGCAGCGCAGG |
| 15180 | G6PD | CCGCAGCGCAGGATGAAGGGC |
| 15190 | G6PD | CAGTATGAGGGCACCTACAAG |
| 15200 | G6PD | CCAGTATGAGGGCACCTACAA |
| 15210 | G6PD | AGCTGGAGAAGCCCAAGCCCA |
| 15220 | G6PD | ACCCCACTGCTGCACCAGATT |
| 15230 | G6PD | CACCCCACTGCTGCACCAGAT |
| 15240 | G6PD | TCACCCCACTGCTGCACCAGA |
| 15250 | G6PD | TGCGGGAGCCAGATGCACTTC |
| 15260 | G6PD | AACCCCGAGGAGTCGGAGCTG |
| 15270 | G6PD | TTCAACCCCGAGGAGTCGGAG |
| 15280 | G6PD | CACCAGCAGTGCAAGCGCAAC |
| 15290 | G6PD | CATGATGTGGCCGGCGACATC |
| 15300 | G6PD | ATCCTGCGCTGCGGCAAGGCC |
| 15310 | G6PD | CGCCACGTAGGGGTGCCCTTC |
| 15320 | G6PD | CCGCCACGTAGGGGTGCCCTT |
| 15330 | KIF11 | ATGAAGATAAATTGATAGCAC |
| 15340 | KIF11 | ATAGCACAAAATCTAGAACTT |
| 15350 | KIF11 | ATGAAACCATAAAAATTGGTT |
| 15360 | KIF11 | GTTTGACTAAGCTTAATTGCT |
| 15370 | KIF11 | GACTAAGCTTAATTGCTTTCT |
| 15380 | KIF11 | ACTAAGCTTAATTGCTTTCTG |
| 15390 | KIF11 | ATTGCTTTCTGGAACAGGATC |
| 15400 | KIF11 | CTTTCTGGAACAGGATCTGAA |
| 15410 | KIF11 | CTGGAACAGGATCTGAAACTG |
| 15420 | KIF11 | TGGAACAGGATCTGAAACTGG |
| 15430 | KIF11 | TCTAATGTCCGTTAAAGGTAC |
| 15440 | KIF11 | AAGGTACGACACCACAGAGGA |
| 15450 | KIF11 | TTTATACCCATCAACACTGGT |
| 15460 | KIF11 | ATACCCATCAACACTGGTAAG |
| 15470 | KIF11 | TACCCATCAACACTGGTAAGA |
| 15480 | KIF11 | ATCAGCTGAAAAGGAAACAGC |
| 15490 | KIF11 | ATGATGCTAAACTGTTCAGAA |
| 15500 | KIF11 | AGAAAACAACAAAGAAGAGAC |
| 15510 | KIF11 | CTTCTTTTAGGATGTGGATGT |
| 15520 | KIF11 | TTCTTTTAGGATGTGGATGTA |
| 15530 | KIF11 | TTTTAGGATGTGGATGTAGAA |
| 15540 | KIF11 | TAGGATGTGGATGTAGAAGAG |
| 15550 | KIF11 | AGGATGTGGATGTAGAAGAGG |
| 15560 | KIF11 | GGATGTGGATGTAGAAGAGGC |
| 15570 | KIF11 | TGGGGCAGTATACTGAAGAAC |
| 15580 | KIF11 | TTCATCAATTGGCGGGGTTCC |
| 15590 | KIF11 | ATCAATTGGCGGGGTTCCATT |
| 15600 | KIF11 | GCGGGGTTCCATTTTTCCAGG |
| 15610 | KIF11 | TCCCGCCTTAAATCCACAGCA |
| 15620 | KIF11 | CCCGCCTTAAATCCACAGCAT |
| 15630 | KIF11 | CCGCCTTAAATCCACAGCATA |
| 15640 | KIF11 | AATCCACAGCATAAAAAATCA |
| 15650 | KIF11 | ACACACTGGAGAGGTCTAAAG |
| 15660 | KIF11 | GTTACAAAGAGCAGATTACCT |
| 15670 | KIF11 | CAAAGAGCAGATTACCTCTGC |
| 15680 | KIF11 | CCTCTGCGAGCCCAGATCAAC |
| 15690 | KIF11 | TAGATTTTGTGCTATCAATTT |
| 15700 | KIF11 | AGTTCTAGATTTTGTGCTATC |
| 15710 | KIF11 | ATTAAGTTCTAGATTTTGTGC |
| 15720 | KIF11 | CATTAAGTTCTAGATTTTGTG |
| 15730 | KIF11 | TGGTTTCATTAAGTTCTAGAT |
| 15740 | KIF11 | ATGGTTTCATTAAGTTCTAGA |
| 15750 | KIF11 | TATGGTTTCATTAAGTTCTAG |
| 15760 | KIF11 | TTATGGTTTCATTAAGTTCTA |
| 15770 | KIF11 | GTCAAACCAATTTTTATGGTT |
| 15780 | KIF11 | AGCTTAGTCAAACCAATTTTT |
| 15790 | KIF11 | CAGAAAGCAATTAAGCTTAGT |
| 15800 | KIF11 | AGATCCTGTTCCAGAAAGCAA |
| 15810 | KIF11 | CAGATCCTGTTCCAGAAAGCA |
| 15820 | KIF11 | GGATATCCAGTTTCAGATCCT |
| 15830 | KIF11 | AAGTACCTGTTGGGATATCCA |
| 15840 | KIF11 | AAAGTACCTGTTGGGATATCC |
| 15850 | KIF11 | TAAAGTACCTGTTGGGATATC |
| 15860 | KIF11 | TCTTTTAAAGTACCTGTTGGG |
| 15870 | KIF11 | CTCTTTTAAAGTACCTGTTGG |
| 15880 | KIF11 | TATTTCTCTTTTAAAGTACCT |
| 15890 | KIF11 | ATGGGTATAAATAACTTTTCC |
| 15900 | KIF11 | CCAGTGTTGATGGGTATAAAT |
| 15910 | KIF11 | TTACCAGTGTTGATGGGTATA |
| 15920 | KIF11 | AGTTCTTACCAGTGTTGATGG |
| 15930 | KIF11 | ACGTGGTTCAGTTCTTACCAG |
| 15940 | KIF11 | AGCTGATCAAGGAGATGTTCA |
| 15950 | KIF11 | CAGCTGATCAAGGAGATGTTC |
| 15960 | KIF11 | TCAGCTGATCAAGGAGATGTT |
| 15970 | KIF11 | CTTTTCAGCTGATCAAGGAGA |
| 15980 | KIF11 | CCTTTTCAGCTGATCAAGGAG |
| 15990 | KIF11 | ACAGCTCAGGCTGTTTCCTTT |
| 16000 | KIF11 | GCATCATTAACAGCTCAGGCT |
| 16010 | KIF11 | AGCATCATTAACAGCTCAGGC |
| 16020 | KIF11 | TGAACAGTTTAGCATCATTAA |
| 16030 | KIF11 | CTGAACAGTTTAGCATCATTA |
| 16040 | KIF11 | TCTGAACAGTTTAGCATCATT |
| 16050 | KIF11 | TTTTCTGAACAGTTTAGCATC |
| 16060 | KIF11 | TTGTTTTCTGAACAGTTTAGC |
| 16070 | KIF11 | TTTGTTGTTTTCTGAACAGTT |
| 16080 | KIF11 | TCTCTTCTTTGTTGTTTTCTG |
| 16090 | KIF11 | CCGGAATTGTCTCTTCTTTGT |
| 16100 | KIF11 | ACCGGAATTGTCTCTTCTTTG |
| 16110 | KIF11 | AATTTACCGGAATTGTCTCTT |
| 16120 | KIF11 | AAATTTACCGGAATTGTCTCT |
| 16130 | KIF11 | AGTATACTGCCCCAGAACTGC |
| 16140 | KIF11 | TTCAGTATACTGCCCCAGAAC |
| 16150 | KIF11 | GAGGTTCTTCAGTATACTGCC |
| 16160 | KIF11 | ACTTAGAGGTTCTTCAGTATA |
| 16170 | KIF11 | ATGAACAATCCACACCAGCAT |
| 16180 | KIF11 | TCTGATATGACATACCTGGAA |
| 16190 | KIF11 | TCTTTTCCATGTGATTTTTTA |
| 16200 | KIF11 | GTCTTTTCCATGTGATTTTTT |
| 16210 | KIF11 | TTTGTCTTTTCCATGTGATTT |
| 16220 | KIF11 | CTTTGTCTTTTCCATGTGATT |
| 16230 | KIF11 | TCTTTGTCTTTTCCATGTGAT |
| 16240 | KIF11 | ATGCCTCTGTTTTCTTTGTCT |
| 16250 | KIF11 | GACCTCTCCAGTGTGTTAATG |
| 16260 | KIF11 | AGACCTCTCCAGTGTGTTAAT |
| 16270 | KIF11 | CACTTTAGACCTCTCCAGTGT |
| 16280 | KIF11 | TTCCACTTTAGACCTCTCCAG |
| 16290 | KIF11 | CTTCCACTTTAGACCTCTCCA |
| 16300 | KIF11 | TAACCAAGTGCTCTGTAGTTT |
| 16310 | KIF11 | GTAACCAAGTGCTCTGTAGTT |
| 16320 | KIF11 | ATCTGGGCTCGCAGAGGTAAT |
| 16330 | KIF11 | AAGGTTGATCTGGGCTCGCAG |
| 16340 | KIF11 | CCAACCCCCAAGTGAATTAAA |
| — | — | — |

TABLE 25
SpyCas9 guide RNAs

| SEQ | | Target Domain Sequence |
|---|---|---|
| ID NO | Gene | (DNA) |
| 16350 | GAPDH | TCTAGGTATGACAACGAATT |
| 16360 | GAPDH | AGCCCCAGCGTCAAAGGTGG |
| 16370 | TBP | ATTGTATCCACAGTGAATCT |
| 16380 | TBP | AAACGCCGAATATAATCCCA |
| 16390 | TBP | ACCATTGTAGCGGTTTGCTG |
| 16400 | TBP | GGTTTGCTGCGGTAATCATG |
| 16410 | TBP | GATAAGAGAGCCACGAACCA |
| 16420 | TBP | ACGGCACTGATTTTCAGTTC |
| 16430 | TBP | CGGCACTGATTTTCAGTTCT |
| 16440 | TBP | GATTTTCAGTTCTGGGAAAA |
| 16450 | TBP | TCTGGGAAAATGGTGTGCAC |
| 16460 | TBP | TGGTGTGCACAGGAGCCAAG |
| 16470 | TBP | TAGTGAAGAACAGTCCAGAC |
| 16480 | TBP | TGCTAGAGTTGTACAGAAGT |
| 16490 | TBP | GCTAGAGTTGTACAGAAGTT |
| 16500 | TBP | GGGTTTTCCAGCTAAGTTCT |
| 16510 | TBP | GGACTTCAAGATTCAGAATA |
| 16520 | TBP | CTTCAAGATTCAGAATATGG |
| 16530 | TBP | TTCAAGATTCAGAATATGGT |
| 16540 | TBP | TCAAGATTCAGAATATGGTG |
| 16550 | TBP | GTGATGTGAAGTTTCCTATA |
| 16560 | TBP | AAGTTTCCTATAAGGTTAGA |
| 16570 | TBP | TCACCCACCAACAATTTAGT |
| 16580 | TBP | TATGAGCCAGAGTTATTTCC |
| 16590 | TBP | GTTCTCCTTATTTTTGTTTC |
| 16600 | TBP | TCTGGAAAAGTTGTATTAAC |
| 16610 | TBP | AAACATCTACCCTATTCTAA |
| 16620 | TBP | ACCCTATTCTAAAGGGATTC |
| 16630 | TBP | GATTCAGGAAGACGACGTAA |
| 16640 | TBP | CACGAAGTGCAATGGTCTTT |
| 16650 | TBP | GTTTCGGGCACGAAGTGCAA |
| 16660 | TBP | GGGATTATATTCGGCGTTTC |
| 16670 | TBP | TGGGATTATATTCGGCGTTT |
| 16680 | TBP | TCTAACCTTGGGATTATATT |
| 16690 | TBP | ATTAAAATAGATCTAACCTT |
| 16700 | TBP | AAAATCAGTGCCGTGGTTCG |
| 16710 | TBP | AGAACTGAAAATCAGTGCCG |
| 16720 | TBP | AATTTCTTACGGCTACCTCT |
| 16730 | TBP | AGTCTGGACTGTTCTTCACT |
| 16740 | TBP | ATATTTTCTTGCTGCCAGTC |
| 16750 | TBP | TTGAAGTCCAAGAACTTAGC |
| 16760 | TBP | ACAAGGCCTTCTAACCTTAT |
| 16770 | TBP | ATTGTTGGTGGGTGAGCACA |
| 16780 | TBP | TTACCTACTAAATTGTTGGT |
| 16790 | TBP | CTTACCTACTAAATTGTTGG |
| 16800 | TBP | AGACTTACCTACTAAATTGT |
| 16810 | TBP | ATTAAACCAGGAAATAACTC |
| 16820 | TBP | ATCATTCTGTAGATTAAACC |
| 16830 | TBP | AAAATAAGGAGAACAATTCT |
| 16840 | TBP | CTTTTCCAGAAACAAAAATA |
| 16850 | TBP | TCCTGAATCCCTTTAGAATA |
| 16860 | TBP | TTCCTGAATCCCTTTAGAAT |
| 16870 | E2F4 | CTCACTCCCACTGCTGTCCC |
| 16880 | E2F4 | CCCTGGCAGTGCAGAAGTCC |
| 16890 | E2F4 | CCTGGCAGTGCAGAAGTCCA |
| 16900 | E2F4 | CAGTGCAGAAGTCCAGGGAA |
| 16910 | E2F4 | GCAGAAGTCCAGGGAATGGC |
| 16920 | E2F4 | GGCCCAGCAGCTGAGATCAC |
| 16930 | E2F4 | GGGGCTATCATTGTAGTGAG |
| 16940 | E2F4 | GCTATCATTGTAGTGAGTGG |
| 16950 | E2F4 | ATTGTAGTGAGTGGCGGCCC |
| 16960 | E2F4 | TTGTAGTGAGTGGCGGCCCT |
| 16970 | E2F4 | CGGCCCTGGGACTGATAGCA |
| 16980 | E2F4 | GGGACTGATAGCAAGGACAG |
| 16990 | E2F4 | TGAGCTCAGTTCACTCCCAC |
| 17000 | E2F4 | GAGCTCAGTTCACTCCCACT |
| 17010 | E2F4 | CCCACTGGGCCCAACAACAC |
| 17020 | E2F4 | GCCCAACAACACTGGACACC |
| 17030 | E2F4 | ACTGCAGTCTTCTGCCCTGC |
| 17040 | E2F4 | AGTAACAGCAGCAGTTCGTC |
| 17050 | E2F4 | TACCTCCTTTGAGCCCATCA |
| 17060 | E2F4 | CCCATCAAGGCAGACCCCAC |
| 17070 | E2F4 | ATCAAGGCAGACCCCACAGG |
| 17080 | E2F4 | GAAATCTTTGATCCCACACG |
| 17090 | E2F4 | TCTTTGATCCCACACGAGGT |
| 17100 | E2F4 | ATTCCCAGAGTGCATGAGCT |
| 17110 | E2F4 | GTGCATGAGCTCGGAGCTGC |
| 17120 | E2F4 | GAGGAGTTGATGTCCTCAGA |
| 17130 | E2F4 | GAGTTGATGTCCTCAGAAGG |
| 17140 | E2F4 | AGTTGATGTCCTCAGAAGGT |
| 17150 | E2F4 | GCTTCGTCTTTCTCCACCCC |
| 17160 | E2F4 | CTTCGTCTTTCTCCACCCCC |
| 17170 | E2F4 | CCACGATTATATCTACAACC |
| 17180 | E2F4 | TACAACCTGGACGAGAGTGA |
| 17190 | E2F4 | GCACTGCCAGGGACAGCAGT |
| 17200 | E2F4 | TGCACTGCCAGGGACAGCAG |
| 17210 | E2F4 | CCTGGACTTCTGCACTGCCA |
| 17220 | E2F4 | CCCTGGACTTCTGCACTGCC |
| 17230 | E2F4 | CTGCTGGGCCAGCCATTCCC |
| 17240 | E2F4 | TGTCCTTGCTATCAGTCCCA |
| 17250 | E2F4 | CTGTCCTTGCTATCAGTCCC |
| 17260 | E2F4 | CCAGTGTTGTTGGGCCCAGT |
| 17270 | E2F4 | TCCAGTGTTGTTGGGCCCAG |
| 17280 | E2F4 | GCCGGGTGTCCAGTGTTGTT |
| 17290 | E2F4 | GGCCGGGTGTCCAGTGTTGT |
| 17300 | E2F4 | AGCAGGGCAGAAGACTGCAG |
| 17310 | E2F4 | GCTGCTGCTGCTGTCCAGCA |
| 17320 | E2F4 | GGAGGTAGAAGGGTTGGGTC |
| 17330 | E2F4 | TGGGCTCAAAGGAGGTAGAA |
| 17340 | E2F4 | ATGGGCTCAAAGGAGGTAGA |
| 17350 | E2F4 | TGCCTTGATGGGCTCAAAGG |
| 17360 | E2F4 | GTCTGCCTTGATGGGCTCAA |
| 17370 | E2F4 | CCTGTGGGGTCTGCCTTGAT |
| 17380 | E2F4 | ACCTGTGGGGTCTGCCTTGA |
| 17390 | E2F4 | GCAGGTACTCACCACCTGTG |
| 17400 | E2F4 | GGCAGGTACTCACCACCTGT |
| 17410 | E2F4 | GGGCAGGTACTCACCACCTG |
| 17420 | E2F4 | AGATTTCTGACAGCTCTTTG |
| 17430 | E2F4 | AAGATTTCTGACAGCTCTTT |
| 17440 | E2F4 | AAAGATTTCTGACAGCTCTT |
| 17450 | E2F4 | TGCAGCAGCCTACCTCGTGT |
| 17460 | E2F4 | ATGCAGCAGCCTACCTCGTG |
| 17470 | E2F4 | GCTCCGAGCTCATGCACTCT |
| 17480 | E2F4 | AGCTCCGAGCTCATGCACTC |
| 17490 | E2F4 | CCAGGGCCACCCACCTTCTG |
| 17500 | E2F4 | TGGAGAAAGACGAAGCAGAG |
| 17510 | E2F4 | GTGGAGAAAGACGAAGCAGA |
| 17520 | E2F4 | GGTGGAGAAAGACGAAGCAG |
| 17530 | E2F4 | TAATCGTGGTCTCCCGGGGG |
| 17540 | E2F4 | ATATAATCGTGGTCTCCCGG |
| 17550 | E2F4 | GATATAATCGTGGTCTCCCG |
| 17560 | E2F4 | AGATATAATCGTGGTCTCCC |
| 17570 | E2F4 | TAGATATAATCGTGGTCTCC |
| 17580 | E2F4 | CCAGGTTGTAGATATAATCG |
| 17590 | E2F4 | AGACACCTTCACTCTCGTCC |
| 17600 | E2F4 | TGAGAACAGGCACATCAAAG |
| 17610 | G6PD | GTGGGGGTTCACCCACTTGT |
| 17620 | G6PD | ACTTGTAGGTGCCCTCATAC |
| 17630 | G6PD | CATCAGCTCGTCTGCCTCCG |
| 17640 | G6PD | ATCAGCTCGTCTGCCTCCGT |
| 17650 | G6PD | TCAGCTCGTCTGCCTCCGTG |
| 17660 | G6PD | CGTCTGCCTCCGTGGGGCCT |
| 17670 | G6PD | TGCCTCCGTGGGGCCTCGGC |
| 17680 | G6PD | TCCTCACCTGCCATAAATAT |
| 17690 | G6PD | CCTCACCTGCCATAAATATA |
| 17700 | G6PD | CTCACCTGCCATAAATATAG |
| 17710 | G6PD | CCTGCCATAAATATAGGGGA |
| 17720 | G6PD | CTGCCATAAATATAGGGGAT |
| 17730 | G6PD | ATAAATATAGGGGATGGGCT |
| 17740 | G6PD | TAAATATAGGGGATGGGCTT |
| 17750 | G6PD | TGGGCTTCTCCAGCTCAATC |
| 17760 | G6PD | AGCTCAATCTGGTGCAGCAG |
| 17770 | G6PD | GCTCAATCTGGTGCAGCAGT |
| 17780 | G6PD | CTCAATCTGGTGCAGCAGTG |
| 17790 | G6PD | CAGTGGGGTGAAAATACGCC |
| 17800 | G6PD | TGAAAATACGCCAGGCCTCA |
| 17810 | G6PD | CCTCACGGAGCTCGTCGCTG |
| 17820 | G6PD | ACCTGCGCACGAAGTGCATC |
| 17830 | G6PD | GGCTCCCGCAGAAGACGTCC |
| 17840 | G6PD | CGCAGAAGACGTCCAGGATG |
| 17850 | G6PD | GTCCAGGATGAGGCGCTCAT |
| 17860 | G6PD | ATGAGGCGCTCATAGGCGTC |
| 17870 | G6PD | TGAGGCGCTCATAGGCGTCA |
| 17880 | G6PD | CACCTTGTATCTGTTGCCGT |
| 17890 | G6PD | TGTATCTGTTGCCGTAGGTC |
| 17900 | G6PD | CAGGTCCAGCTCCGACTCCT |
| 17910 | G6PD | AGGTCCAGCTCCGACTCCTC |
| 17920 | G6PD | GGTCCAGCTCCGACTCCTCG |
| 17930 | G6PD | TCGGGGTTGAAGAACATGCC |
| 17940 | G6PD | GAAGAACATGCCCGGCTTCT |
| 17950 | G6PD | CGGCTTCTTGGTCATCATCT |
| 17960 | G6PD | GGTCATCATCTTGGTGTACA |
| 17970 | G6PD | CTTGGTGTACACGGCCTCGT |
| 17980 | G6PD | TTGGTGTACACGGCCTCGTT |
| 17990 | G6PD | CGGCCTCGTTGGGCTGCACG |
| 18000 | G6PD | GCTCGTTGCGCTTGCACTGC |
| 18010 | G6PD | CGTTGCGCTTGCACTGCTGG |
| 18020 | G6PD | CTGCTGGTGGAAGATGTCGC |
| 18030 | G6PD | AGATGTCGCCGGCCACATCA |
| 18040 | G6PD | ATGGAACTGCAGCCTCACCT |
| 18050 | G6PD | CCTCGGCCTTGCGCTCGTTC |
| 18060 | G6PD | CTCGGCCTTGCGCTCGTTCA |
| 18070 | G6PD | TCAGGGCCTTGCCGCAGCGC |
| 18080 | G6PD | CTTGCCGCAGCGCAGGATGA |
| 18090 | G6PD | TTGCCGCAGCGCAGGATGAA |
| 18100 | G6PD | GTATGAGGGCACCTACAAGT |
| 18110 | G6PD | AGTATGAGGGCACCTACAAG |
| 18120 | G6PD | AGAGTGGGTTTCCAGTATGA |
| 18130 | G6PD | GAGAGTGGGTTTCCAGTATG |
| 18140 | G6PD | GACGAGCTGATGAAGAGAGT |
| 18150 | G6PD | AGACGAGCTGATGAAGAGAG |
| 18160 | G6PD | CTCCAGCCGAGGCCCCACGG |
| 18170 | G6PD | CACCCGTCACTCTCCAGCCG |
| 18180 | G6PD | CCATCCCCTATATTTATGGC |
| 18190 | G6PD | AAGCCCATCCCCTATATTTA |
| 18200 | G6PD | ACTGCTGCACCAGATTGAGC |
| 18210 | G6PD | GCGACGAGCTCCGTGAGGCC |
| 18220 | G6PD | CCTCAGCGACGAGCTCCGTG |
| 18230 | G6PD | GCCAGATGCACTTCGTGCGC |
| 18240 | G6PD | TCATCCTGGACGTCTTCTGC |
| 18250 | G6PD | CTCATCCTGGACGTCTTCTG |
| 18260 | G6PD | CGCCTATGAGCGCCTCATCC |
| 18270 | G6PD | GACCTACGGCAACAGATACA |
| 18280 | G6PD | TCGGAGCTGGACCTGACCTA |
| 18290 | G6PD | CAACCCCGAGGAGTCGGAGC |
| 18300 | G6PD | GTTCTTCAACCCCGAGGAGT |
| 18310 | G6PD | GGGCATGTTCTTCAACCCCG |
| 18320 | G6PD | AAGATGATGACCAAGAAGCC |
| 18330 | G6PD | CAAGATGATGACCAAGAAGC |
| 18340 | G6PD | GATCCGCGTGCAGCCCAACG |
| 18350 | G6PD | GCAGTGCAAGCGCAACGAGC |
| 18360 | G6PD | CTGCAGTTCCATGATGTGGC |
| 18370 | G6PD | GAGGCTGCAGTTCCATGATG |
| 18380 | G6PD | ACGAGCGCAAGGCCGAGGTG |
| 18390 | G6PD | CCTGAACGAGCGCAAGGCCG |
| 18400 | G6PD | CAAGGCCCTGAACGAGCGCA |
| 18410 | G6PD | CTTCATCCTGCGCTGCGGCA |
| 18420 | G6PD | GTGCCCTTCATCCTGCGCTG |
| 18430 | G6PD | AGAATGAGAGGTGGGATGGT |
| 18440 | G6PD | GTGGAGAATGAGAGGTGGGA |
| 18450 | KIF11 | CTTAATGAAACCATAAAAAT |
| 18460 | KIF11 | GACTAAGCTTAATTGCTTTC |
| 18470 | KIF11 | GCTTAATTGCTTTCTGGAAC |
| 18480 | KIF11 | TCTGGAACAGGATCTGAAAC |
| 18490 | KIF11 | CTGAAACTGGATATCCCAAC |
| 18500 | KIF11 | TTAAAGGTACGACACCACAG |
| 18510 | KIF11 | TTATTTATACCCATCAACAC |
| 18520 | KIF11 | ATCTCCTTGATCAGCTGAAA |
| 18530 | KIF11 | CAACAAAGAAGAGACAATTC |
| 18540 | KIF11 | TTAGGATGTGGATGTAGAAG |
| 18550 | KIF11 | GGATGTAGAAGAGGCAGTTC |
| 18560 | KIF11 | GATGTAGAAGAGGCAGTTCT |
| 18570 | KIF11 | ATGTAGAAGAGGCAGTTCTG |
| 18580 | KIF11 | CAAGAGCCATCTGTAGATGC |
| 18590 | KIF11 | GCCATCTGTAGATGCTGGTG |
| 18600 | KIF11 | GGTGTGGATTGTTCATCAAT |
| 18610 | KIF11 | GTGGATTGTTCATCAATTGG |
| 18620 | KIF11 | TGGATTGTTCATCAATTGGC |
| 18630 | KIF11 | GGATTGTTCATCAATTGGCG |
| 18640 | KIF11 | TGGCGGGGTTCCATTTTTCC |
| 18650 | KIF11 | CCACAGCATAAAAAATCACA |
| 18660 | KIF11 | GGAAAAGACAAAGAAAACAG |
| 18670 | KIF11 | AAACAGAGGCATTAACACAC |
| 18680 | KIF11 | GAGGCATTAACACACTGGAG |
| 18690 | KIF11 | CACACTGGAGAGGTCTAAAG |
| 18700 | KIF11 | GGAAGAAACTACAGAGCACT |
| 18710 | KIF11 | CTTAGTCAAACCAATTTTTA |
| 18720 | KIF11 | TCTCTTTTAAAGTACCTGTT |
| 18730 | KIF11 | TTCTCTTTTAAAGTACCTGT |
| 18740 | KIF11 | TATAAATAACTTTTCCTCTG |
| 18750 | KIF11 | CAGTTCTTACCAGTGTTGAT |
| 18760 | KIF11 | TCAGTTCTTACCAGTGTTGA |
| 18770 | KIF11 | TGATCAAGGAGATGTTCACG |
| 18780 | KIF11 | GTTTCCTTTTCAGCTGATCA |
| 18790 | KIF11 | TTTAGCATCATTAACAGCTC |
| 18800 | KIF11 | ACAGATGGCTCTTGACTTAG |
| 18810 | KIF11 | TCCACACCAGCATCTACAGA |
| 18820 | KIF11 | ATATGACATACCTGGAAAAA |
| 18830 | KIF11 | AGGTTGATCTGGGCTCGCAG |
| 18840 | KIF11 | AGTGAATTAAAGGTTGATCT |
| 18850 | KIF11 | AAGTGAATTAAAGGTTGATC |
| — | — | — |

### Example 18: Generating Edited iPSC Cells Using Cas12a and Testing Effect of Activin A on Pluripotency

To generate natural killer cells from pluripotent stem cells, a representative induced pluripotent stem cell (iPSC) line was generated and designated “PCS-201”. This line was generated by reprogramming adult male human primary dermal fibroblasts purchased from ATCC (ATCCR: PCS-201-012) using a commercially available non-modified RNA reprogramming kit (Stemgent/Reprocell, USA). The reprogramming kit contains non-modified reprogramming mRNAs (OCT4, SOX2, KLF4, cMYC, NANOG, and LIN28) with immune evasion mRNAs (E3, K3, and B18R) and double-stranded microRNAs (miRNAs) from the 302/367 clusters. Fibroblasts were seeded in fibroblast expansion medium (DMEM/F12 with 10% FBS). The next day, media was switched to Nutristem medium and daily overnight transfections were performed for 4 days (day 1 to 4). Primary iPSC colonies appeared on day 7 and were picked on day 10-14. Picked colonies were expanded clonally to achieve a sufficient number of cells to establish a master cell bank. The parental line chosen from this process and used for the subsequent experiments passed standard quality controls, including confirmation of stemness marker expression, normal karyotype and pluripotency.

To generate edited iPSC cells, the PCS-201 (PCS) cells were electroporated with a Cas 12a RNP designed to cut at the target gene of interest. Briefly, the cells were treated 24 hours prior to transfection with a ROCK inhibitor (Y27632). On the day of transfection, a single cell solution was generated using accutase and 500,000 PCS iPS cells were resuspended in the appropriate electroporation buffer and Cas 12a RNP at a final concentration of 2 μM. When two RNPs were added simultaneously, the total RNP concentration was 4 μM (2+2). This solution was electroporated using a Lonza 4D electroporator system. Following electroporation, the cells were plated in 6-well plates in mTESR media containing CloneR (Stemcell Technologies). The cells were allowed to grow for 3-5 days with daily media changes, and the CloneR was removed from the media by 48 hours post electroporation. To pick single colonies, the expanded cells were plated at a low density in 10 cm plates after resuspending them in a single cell suspension. Rock inhibitor was used to support the cells during single cell plating for 3-5 days post plating depending on the size of the colonies on the plate. After 7-10 days, sufficiently sized colonies with acceptable morphology were picked and plated into 24-well plates. The picked colonies were expanded to sufficient numbers to allow harvesting of genomic DNA for subsequent analysis and for cell line cryopreservation. Editing was confirmed by NGS and selected clones were expanded further and banked. Ultimately, karyotyping, stemness flow, and differentiation assays were performed on a subset of selected clones.

Two target genes of interest were CISH and TGFβRII, both of which were hypothesized to enhance natural killer cell function. As the TGFβ:TGFβRII pathway is believed to be involved in the maintenance of pluripotency, it was hypothesized that a functional deletion of TGFβRII in iPSCs could lead to differentiation and prevent generation of TGFβRII edited iPSCs. Due to the convergence of Activin receptor signaling and TGFβRII signaling in regulating SMAD2/3 and other intracellular molecules, it was hypothesized that Activin A could replace TGFβ in commercially available pluripotent stem cell medias to generate edited lines. To test this hypothesis, the pluripotency of unedited and TGFβRII edited iPSCs grown with Activin A was assessed. Several different culture medias were utilized: “E6” (Essential 6TM Medium, #A1516401, ThermoFisher), which lacks TGFβ, “E7”, which was E6 supplemented with 100 ng/ml of bFGF (Peprotech, #100-18B), “E8” (Essential 8™ Medium, #A1517001, ThermoFisher), and “E7+ActA”, which was E6 supplemented with 100 ng/ml of bFGF and varying concentrations of Activin A (Peprotech #120-14P). Typically, E6 and E7 medias are typically insufficient to maintain the stemness and pluripotency of PSCs over multiple passages in culture.

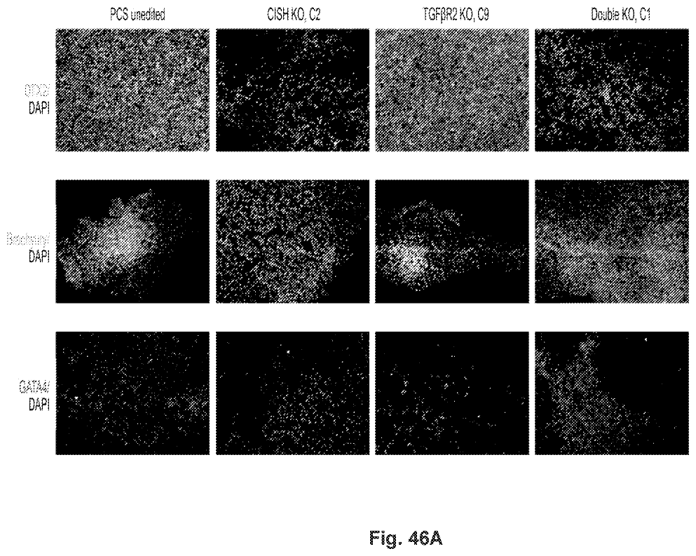

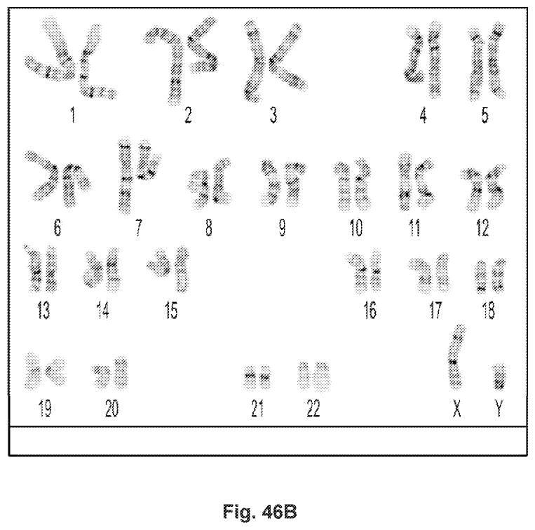

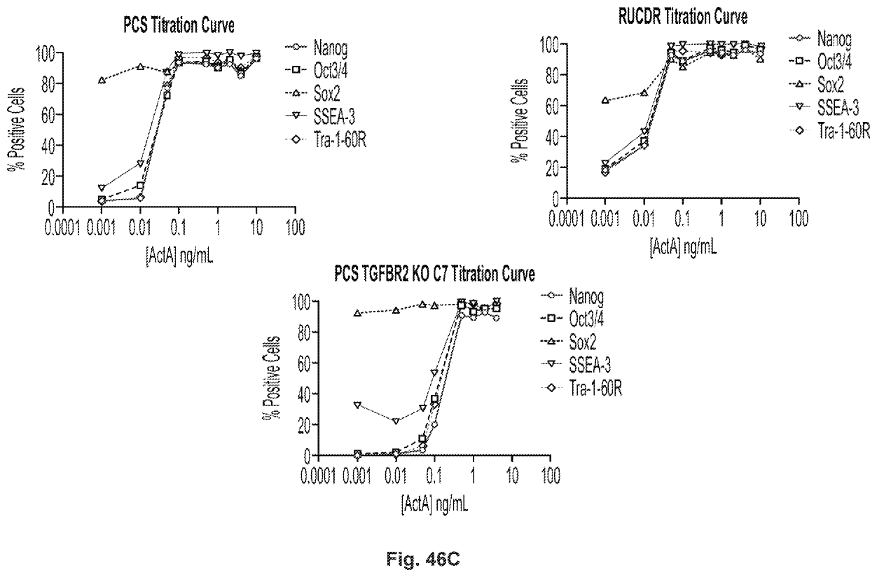

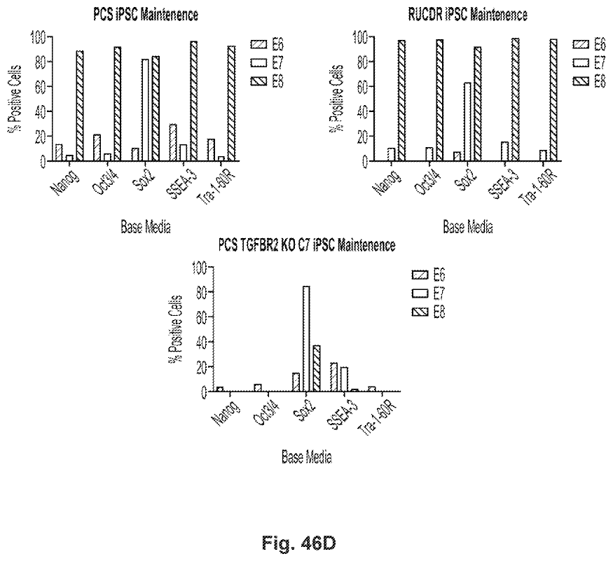

In order to determine whether Activin A could maintain PCS iPSCs in the absence of exogenous TGFβ, unedited PCS iPSCs were plated on a LaminStem™ 521 (Biological Industries) coated 6-well plate and cultured in E6, E7, E8 or E7+ActA (with Activin A at two different concentrations—1 ng/ml and 4 ng/ml). After 2 passages, the cells were assessed for morphology and stemness marker expression. Morphology was assessed using a standard phase contrast setting on an inverted microscope. Colonies with defined edges and non-differentiated cells typical of iPSC colonies, were deemed to be stem like. To confirm the morphological observations, the expression of standard iPS cell stemness markers was measured using intracellular flow cytometry. Briefly, cells were dissociated, stained for extracellular markers, and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®: Clone TRA-1-60-R), anti-Human Nanog_AF®647 (BD Pharmingen™: Clone N31-355), and anti-Oct4 (Oct3)_PE (Biolegend®: Clone 3A2A20). Cells were recorded on a NovoCyte Quanteon Flow Cytometer (Agilent) and analyzed using FlowJo (FlowJo, LLC). As shown in FIG. 46, both 1 ng/ml and 4 ng/ml of Activin A was sufficient to maintain pluripotency with equivalent stemness marker expression to the cells grown in E8. As expected, cells grown in E6 and E7 (which lacked TGFβ) did not maintain stemness gene expression to the same degree as E8, indicating the loss of iPSC stemness in the absence of TGFβ or Activin A. These results suggest that Activin A can supplement iPSC stemness in the absence of TGFβ signaling.

Given the demonstration that Activin A could support iPSC stemness in the absence of TGFβ, TGFβRII knockout (“KO”) iPSCs, CISH KO iPSCs, and TGFβRII/CISH double knockout (“DKO”) iPSC lines were generated. Specifically, iPSCs were edited using an RNP having an engineered Cas12a with three amino acid substitutions (M537R, F870L, and H800A (SEQ ID NO: 62)) and a gRNA specific for CISH or TGFβRII. To make CISH/TGFβRII DKO iPSCs, iPSCs were treated with an RNP targeting CISH and an RNP targeting TGFβRII simultaneously. The particular guide RNA sequences of Table 26 were used for editing of CISH and TGFβRII. Both guides were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence.

TABLE 26
Guide RNA sequences

| | gRNA | |
|---|---|---|
| | Targeting | |
| | Domain | |
| Target | Sequence | Full Length gRNA Sequence |
| CISH 7050 | GGUGUACA | ATGTGTTTTTGTCAAAAGACCTTTTrUrA |
| | GCAGUGGC | rArUrUrUrCrUrArCrUrCrUrUrGrUr |
| | UGGU | ArGrArUrGrGrUrGrUrArCrArGrCrA |
| | (SEQ ID | rGrUrGrGrCrUrGrGrU (SEQ ID |
| | NO: 1155) | NO: 1156) |
| TGFβRII | UGAUGUGA | ATGTGTTTTTGTCAAAAGACCTTTTrUrA |
| 24026 | GAUUUUC | rArUrUrUrCrUrArCrUrCrUrUrGrUr |
| | CACCU | ArGrArUrUrGrArUrGrUrGrArGrArU |
| | (SEQ ID | rUrUrUrCrCrArCrCrU (SEQ ID |
| | NO: 1157) | NO: 1158) |

The edited clones were generated as described above with a minor modification for the cells treated with TGFβRII RNPs. Briefly, TGFβRII-edited PCS iPSCs and TGFβRII/CISH edited PCS iPSCs were plated after electroporation at the 6-well stage in the mTESR supplemented with 10 ng/ml of Activin A in order to support the generation of edited clones. The cells were cultured with 10 ng/ml of Activin A through the cell colony picking and early expansion stages. Colonies assessed as having the correct single KO (CISH KO or TGFβRII KO) or double KO (CISH/TGFβRII DKO) were picked and expanded (clonal selection).

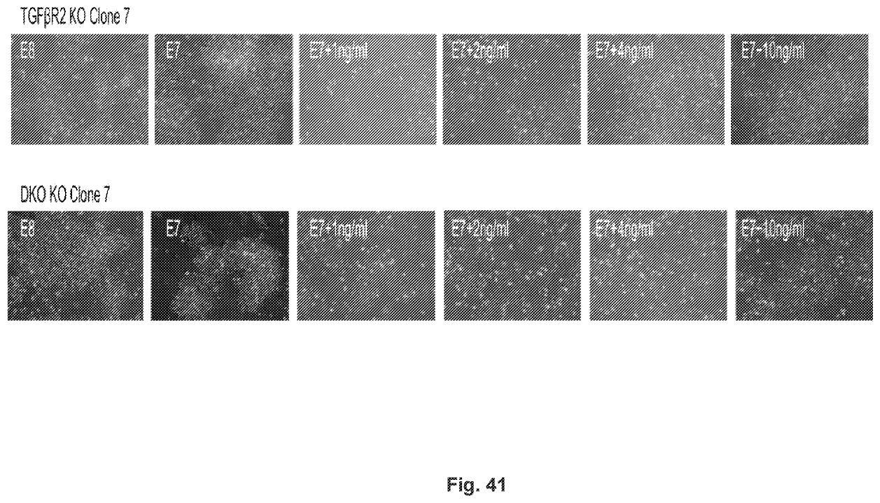

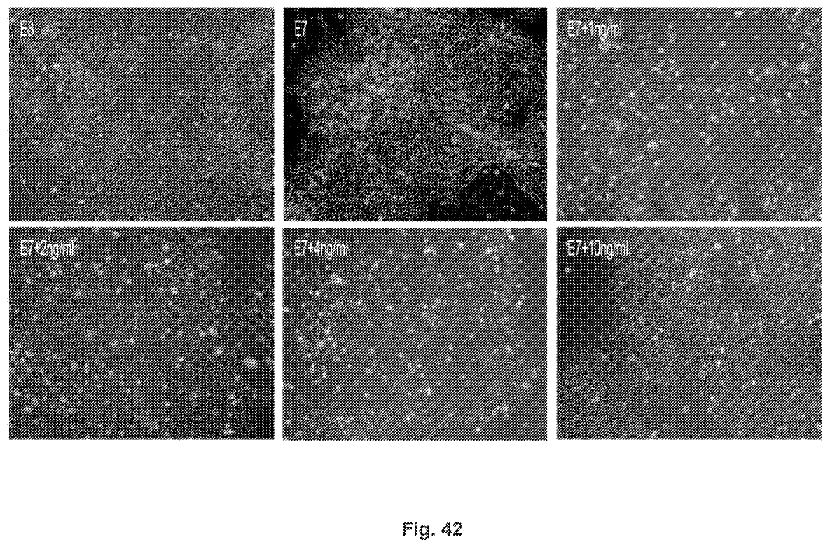

To determine the optimal concentration of Activin A for culturing of TGFβRII KO and TGFβRII/CISH DKO iPSCs, a slightly expanded concentration curve was tested as shown FIG. 41. Similar to the assessment performed previously, the iPSCs were cultured in a Matrigel-treated 6-well plate with concentrations of 1 ng/ml, 2 ng/ml, 4 ng/ml and 10 ng/ml Activin A. As shown in FIG. 41, TGFβRII KO or CISH/TGFβRII DKO cells cultured in E7 medium supplemented with 4 ng/mL Activin A for 19 days (over 5 passages) maintained a wild type morphology. FIG. 42 shows the morphology of TGFβRII KO PCS-201 hiPSC Clone 9.

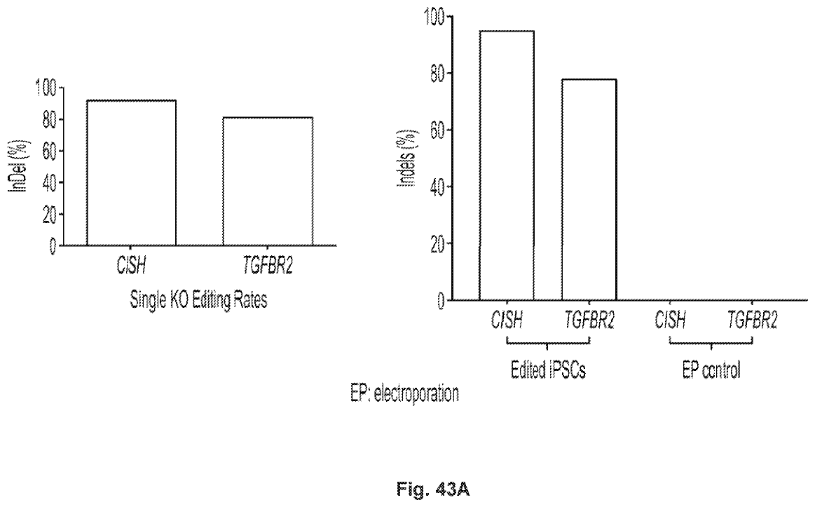

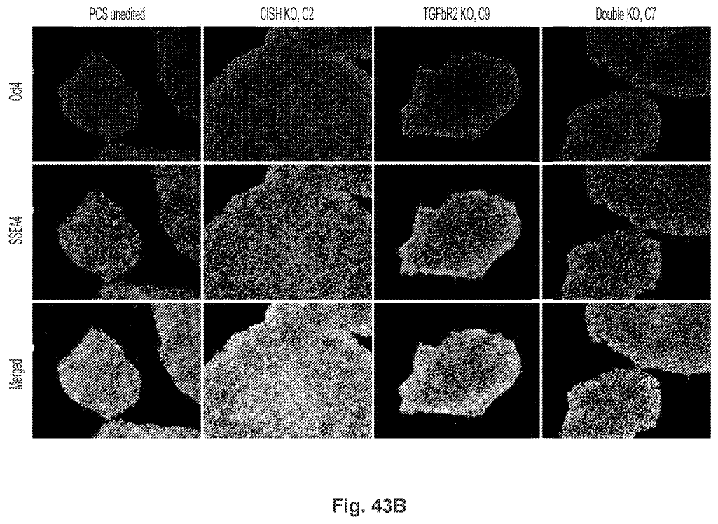

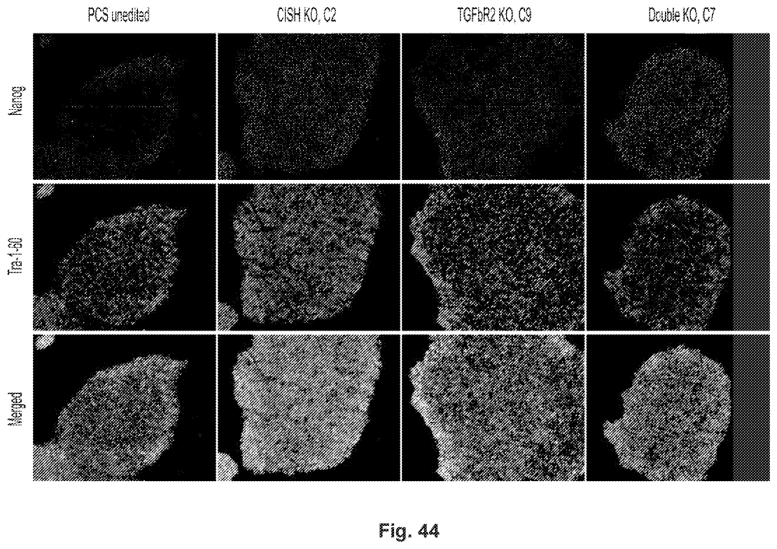

As shown in FIG. 43A, the initial editing efficiency of the iPSCs treated simultaneously with the CISH and TGFβRII RNPs (prior to clonal selection) was high, with 95% of the CISH alleles edited and 78% of the TGFβRII alleles edited. Unedited iPSC controls did not have indels at either loci. iPSCs that were treated with CISH or TGFβRII RNPs individually showed 93% and 82% editing rates prior to clone selection (depicted in FIG. 43A). The KO cell lines (CISH KO iPSCs, TGFβRII KO iPSCs, and CISH/TGFβRII DKO iPSCs) were subsequently assessed for the presence of pluripotency markers Oct4, SSEA4, Nanog, and Tra-1-60 after culturing in the presence of supplemental Activin A. As shown in FIGS. 43B and 44, culturing the KO cell lines in Activin A maintained expression of these pluripotency markers.

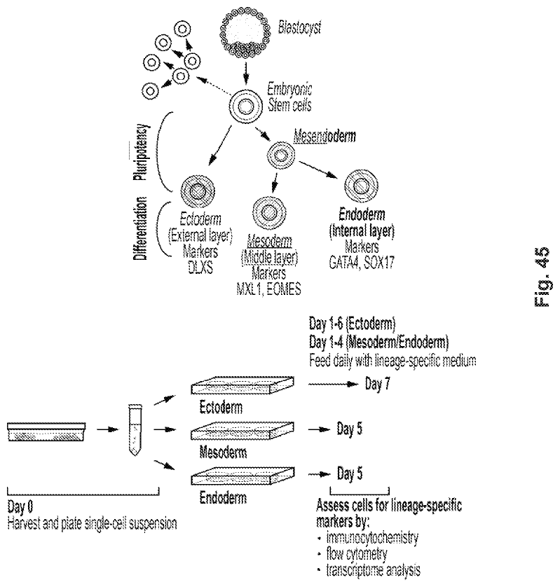

The KO iPSC lines cultured in Activin A were next assessed for their capacity to differentiate using the STEMdiff™ Trilineage Differentiation Kit assay (from STEMCELL Technologies Inc., Vancouver, BC, CA) as depicted schematically in FIG. 45. As shown in FIG. 46A, culturing the single KO (TGFβRII KO iPSCs or CISH KO iPSCs) and DKO (TGFβRII/CISH DKO iPSCs) cell lines in media with supplemental Activin A maintained their ability to differentiate into early progenitors of all 3 germ layers, as shown by expression of ectoderm (OTX2), mesoderm (brachyury), and endoderm (GATA4) markers (FIG. 46A). The unedited PCS control cells were also able to express each of these markers.

The edited iPSCs were next karyotyped to determine whether the Cas12a editing caused large genetic abnormalities, such as translocations. As shown in FIG. 46B, the cells had normal karyotypes with no translocation between the cut sites.

To further support the results described above, an expanded Activin A concentration curve was performed on the unedited parental PSC line, an edited TGFβRII KO iPSC clone (C7), and an additional representative (unedited) cell line designated RUCDR (RUCDR Infinite Biologics group, Piscaway NJ). At the outset, the iPSCs were seeded at 1e5 cells per well in a 1× LaminStem™ 521 (Biological Industries) coated 12-well plate. Cells were then passaged 10 times over ˜40-50 days using 0.5 mM EDTA in 1×PBS dissociation and Y-27632 (Biological Industries) until wells achieved >75% confluency. Cells were cultured in Essential 6TM Medium (Gibco), TeSR™-E7™, and TeSR™-E8™ (StemCell Technologies) for controls and titrated using TeSR™-E7™ supplemented with E, coli-derived recombinant human/murine/rat Activin A (PeproTech) spanning a 4-log concentration dosage (0.001-10 ng/ml). Following 5 and 10 passages, cells were dissociated and then fixed overnight and permeabilized using the reagents and standard protocol from the Foxp3/Transcription Factor Staining Buffer Set (eBioscience™). Cells were stained for flow cytometric analysis with anti-human TRA-1-60-R_AF®488 (Biolegend®; Clone TRA-1-60-R), anti Sox2_PerCP-Cy™5.5 (BD Pharmingen™: Clone 030-678), anti Human Nanog_AF®647 (BD Pharmingen™; Clone N31-355), anti-Oct4 (Oct3)_PE (Biolegend R: Clone 3A2A20), and anti-human SSEA-4_PE/Dazzle™ 594 (Biolegend®; Clone MC-813-70). Cells were recorded on a NovoCyte Quanteon Flow Cytometer (Agilent) and analyzed using FlowJo (FlowJo, LLC). FIG. 46C shows the titration curves for the tested iPSC lines. The minimum concentration of Activin A required to maintain each line varied slightly, with the TGFβRII KO iPSCs requiring a higher baseline amount of Activin A as compared to the parental control (0.5 ng/ml vs 0.1 ng/ml). In all 3 cell lines, 4 ng/ml was well above the minimum amount of Activin A necessary to maintain stemness marker expression over an extended culture period. FIG. 46D shows the stemness marker expression in the cells culture with the base medias alone (no Activin A). As expected, the TGFβRII KO iPSCs did not maintain expression, while the two unedited lines were able to maintain stemness marker expression in E8.

### Example 19: Differentiation of Edited CISH KO, TGFβRII KO, and CISH/TGFβRII DKO iPSCs into iNK Cells Exhibiting Enhanced Function

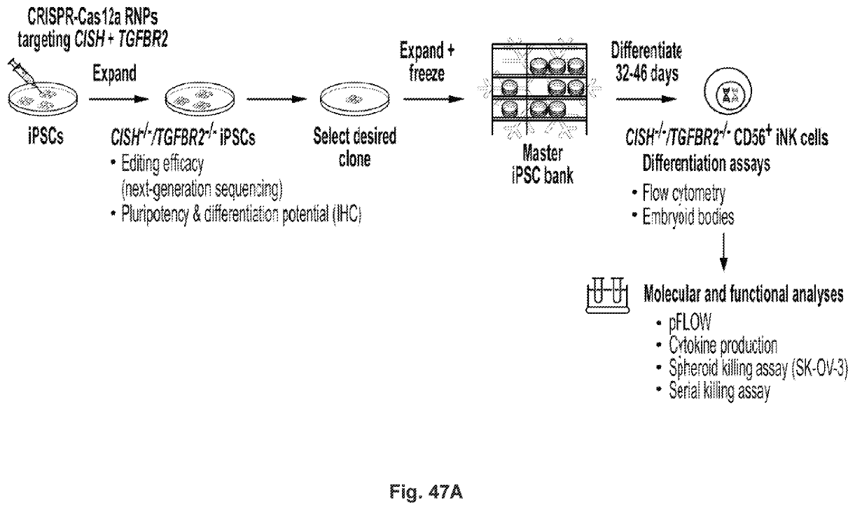

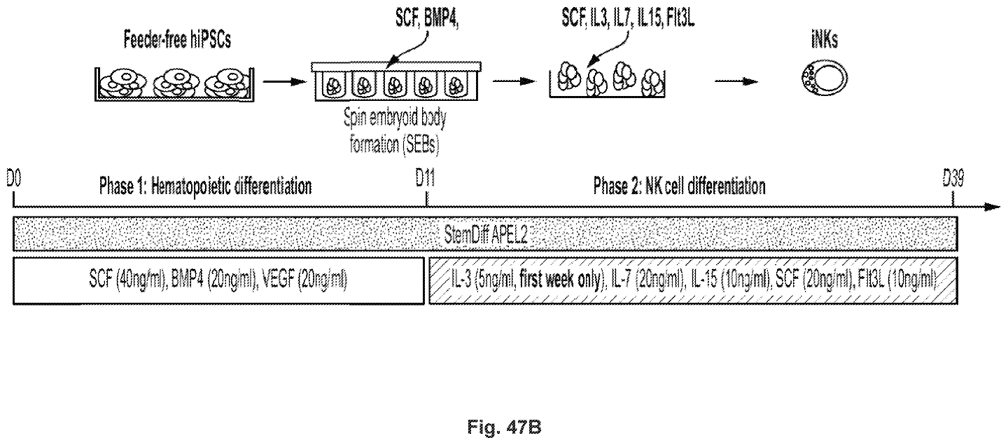

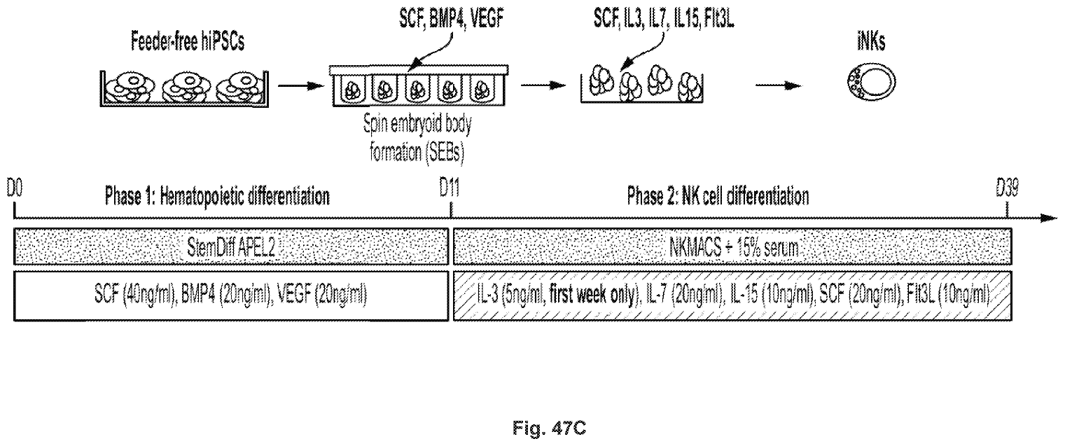

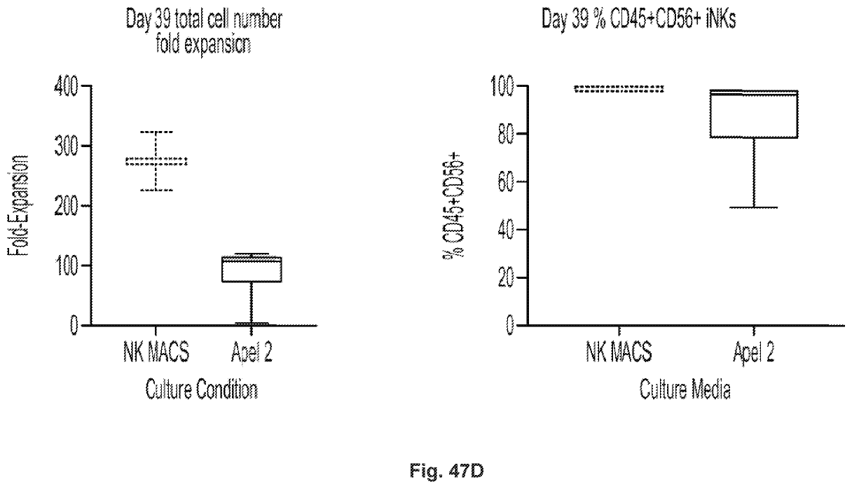

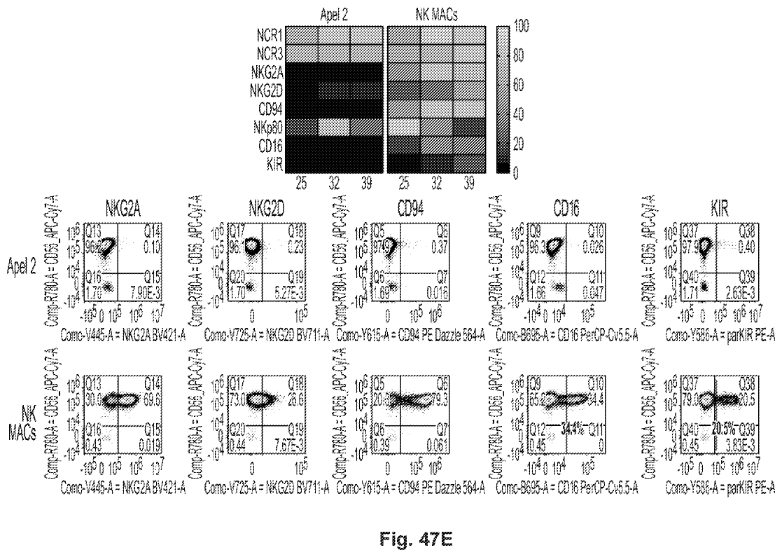

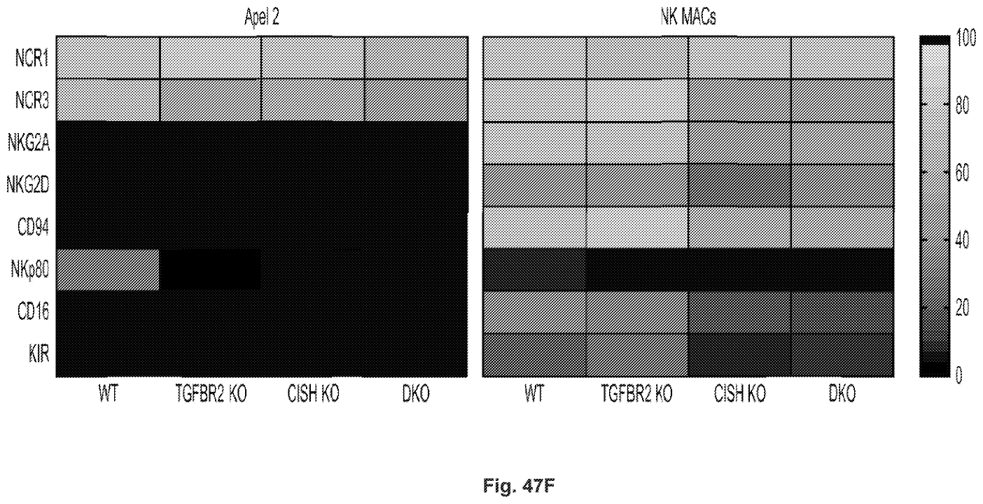

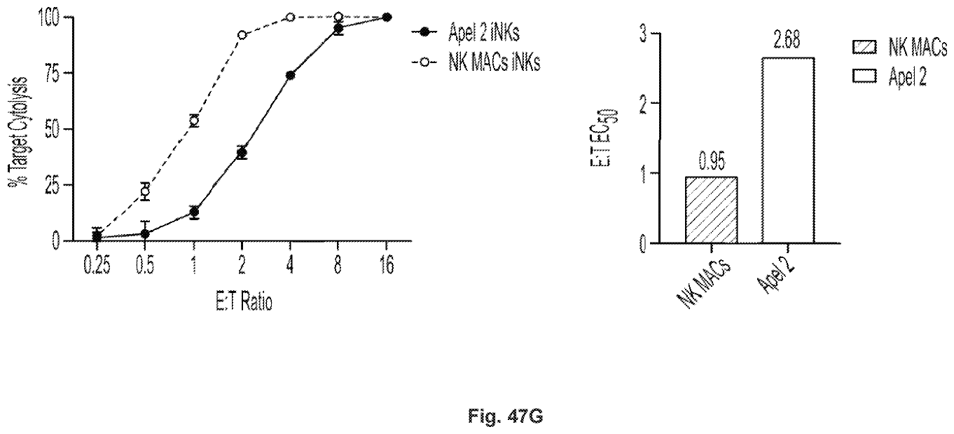

FIG. 47A depicts a schematic of an exemplary workflow for development of a CRISPR-Cas12a-edited iPSC platform for generation of enhanced CD56+ iNK cells. As shown in FIG. 47A. the CISH and TGFβRII genes are targeted in iPSCs via delivery of RNPs to the cells using electroporation to generate CISH/TGFβRII DKO iPSCs. iPSCs with the desired edits at both the CISH and TGFβRII genes can then be selected and expanded to create a master iPSC bank. Edited cells from the iPSC master bank can then be differentiated into CD56+ CISH/TGFβRII DKO iNK cells.

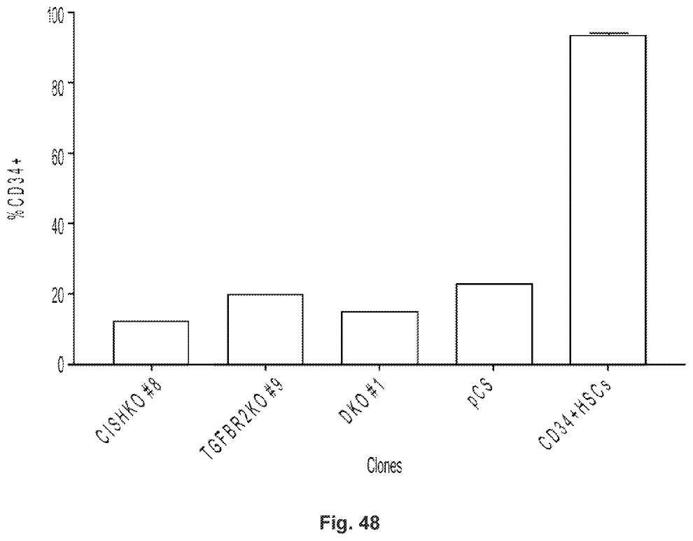

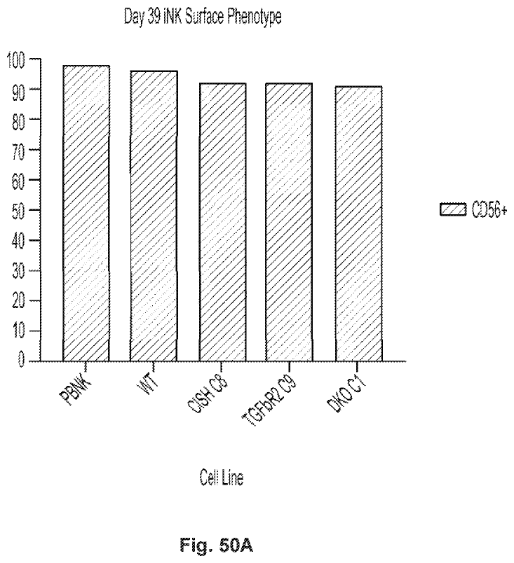

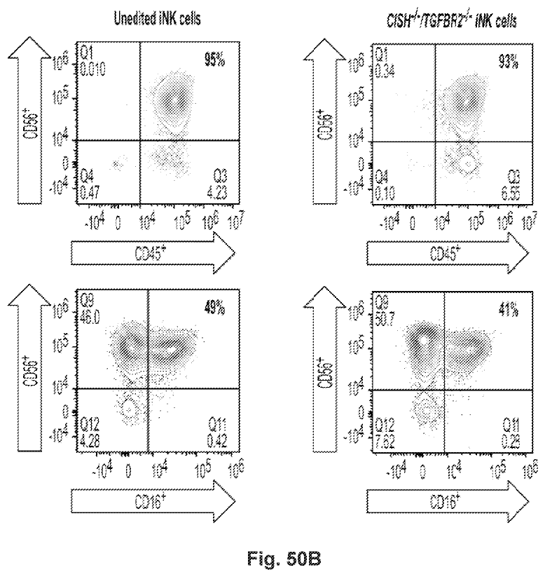

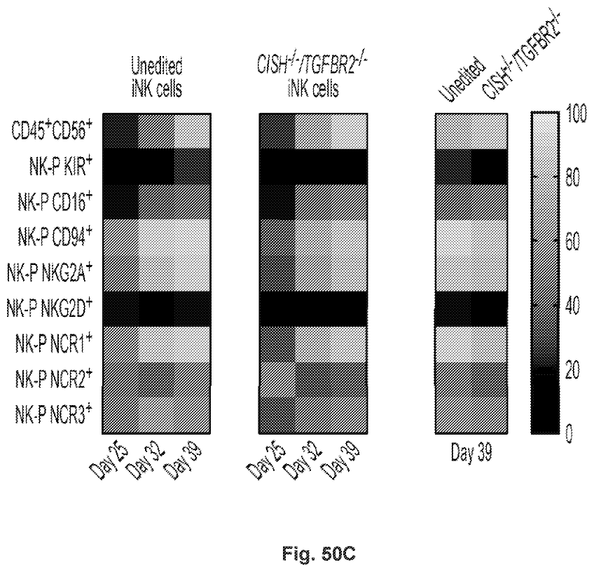

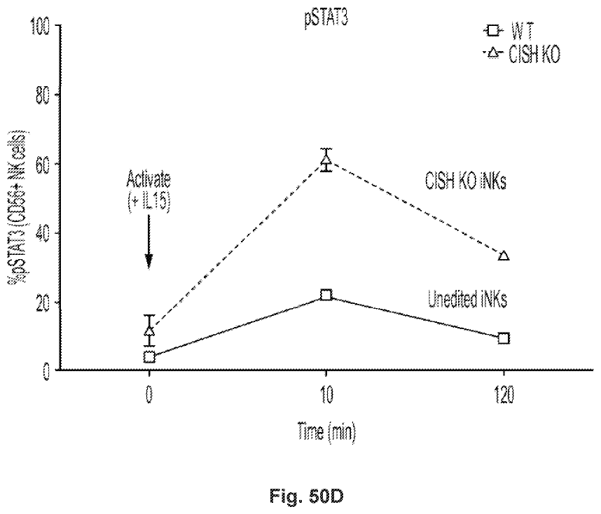

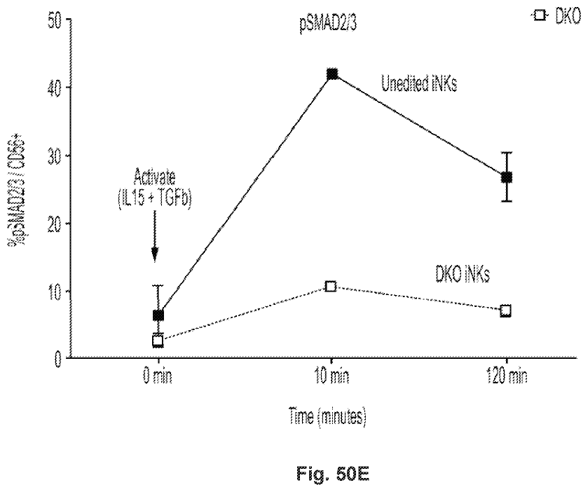

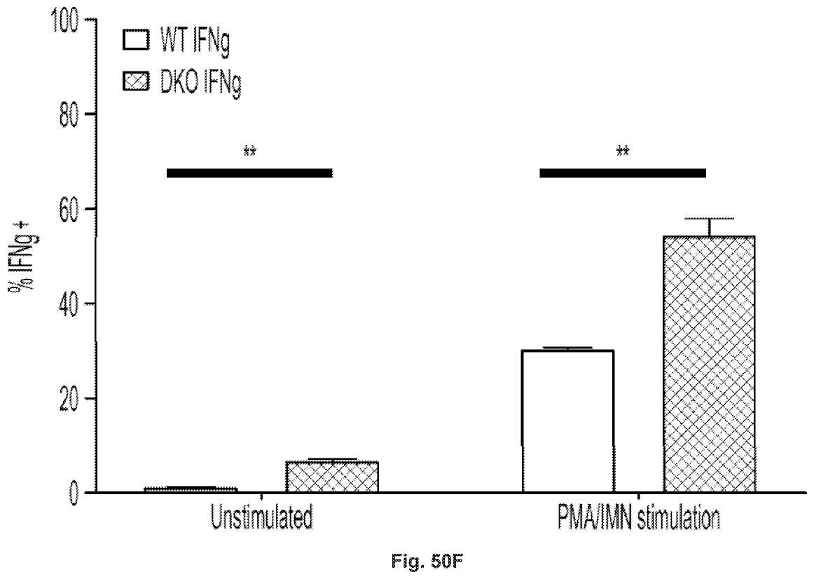

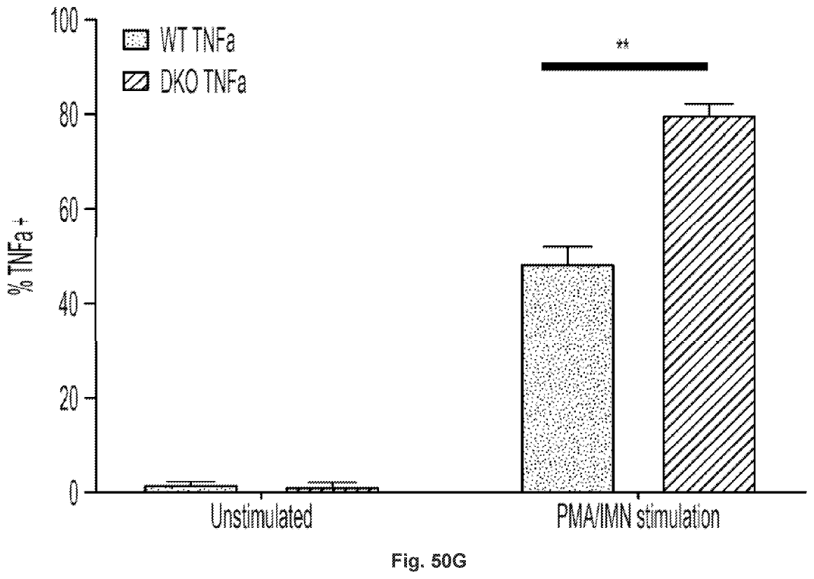

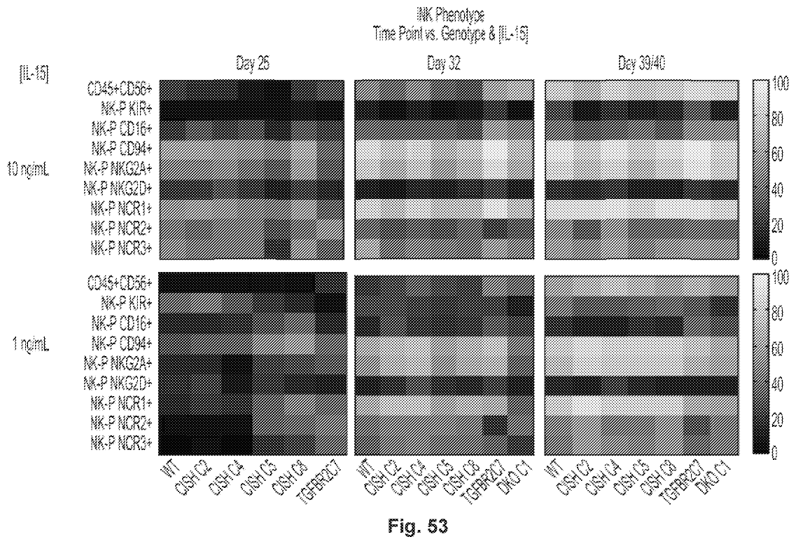

FIGS. 47B and 47C depict two exemplary schematics of the process of differentiating iPSCs into iNK cells. As shown in FIGS. 47B and 47C. edited cells (or unedited control cells) were differentiated using a two-phase process. First, in the “hematopoietic differentiation phase.” hiPSCs (edited and unedited) were cultured in StemDiff™ APEL2TM medium (StemCell Technologies) with SCF (40 ng/mL). BMP4 (20) ng/ml), and VEGF (20 ng/ml) from days 0-10, to produce spin embryoid bodies (SEBs). As shown in FIG. 53B, SEBs were then cultured from days 11-39 in StemDiff™ APEL2™ medium comprising IL-3 (5 ng/mL, only present for the first week of culture), IL-7 (20) ng/mL), IL-15 (10 ng/mL), SCF (20 ng/ml), and Flt3L (10 ng/ml) in an NK cell differentiation phase, CISH KO iPSCs, TGFβRII KO iPSCs, CISH/TGFβRII DKO iPSCs, and unedited wild-type iPSC lines (PCS), were differentiated into iNKs according to the schematic in FIG. 47B, and then characterized to assess whether they exhibited a phenotype congruent with NK cells (see FIGS. 48, 49, and 50A), CISH KO iPSCs, TGFβRII KO iPSCs, CISH/TGFβRII DKO iPSCs, and unedited wild-type iPSC lines, described in FIGS. 50B, 50C, 51B, 51C, and 52 were also differentiated into iNKs utilizing the alternative method shown in FIG. 47C, and then characterized to assess whether they exhibited a phenotype congruent with NK cells (see FIGS. 50B, 50C, 51B, 51C, and 52).

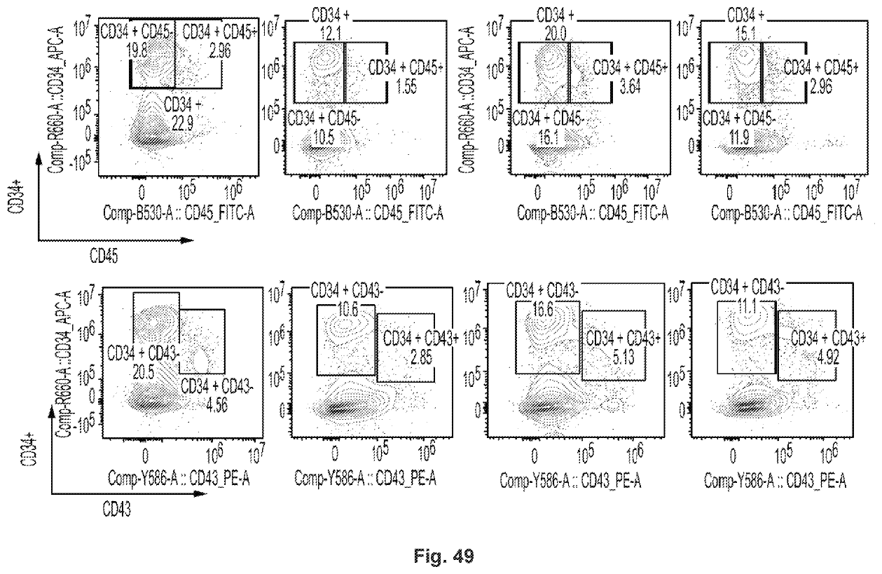

Specifically, the CISH KO INKs, TGFβRII KO INKs, CISH/TGFβRII DKO iNKs were assessed for exemplary phenotypic markers of (i) stem cells (CD34); and (ii) hematopoietic cells (CD43 and CD45) by flow cytometry. Briefly, two rows of embryoid bodies from a 96-well plate for each genotype were harvested for staining. Once a single cell solution was generated using Trypsin and mechanical disruption, the cells were stained for the human markers CD34, CD45, CD31, CD43, CD235a and CD41. As shown in FIG. 48, CISH KO INKs, TGFβRII KO INKs, CISH/TGFβRII DKO iNKs, and iNKs derived from wild-type parental clones (PCS) exhibited lower levels of CD34 relative to control cells, which were purified CD34+ HSCs. CD34 expression levels were similar across these iNK cell clones indicating that editing of the iPSCs did not affect differentiation to the CD34+ stage. FIG. 49 shows that CISH KO iNKs, TGFβRII KO INKs, CISH/TGFβRII DKO INKs, and iNKs derived from wild-type parental clones (PCS) exhibited similar surface expression profiles for CD43 and CD45. Thus, iNKs differentiated from edited and unedited iPSCs exhibited similar levels of markers for stem cells and hematopoietic cells, and both differentiated edited and unedited cells exhibited certain NK cell phenotypes based on marker expression profiles.

CISH KO INKs, TGFβRII KO INKs, CISH/TGFβRII DKO iNKs, iNKs derived from wild-type parental clones (WT), and NK cells derived from peripheral blood (PBNKs) were further assayed to determine their surface expression of CD56, a marker for NK cells. Briefly, cells were harvested on day 39 of differentiation, washed and resuspended in a flow staining buffer containing antibodies that recognize human CD56, CD16, NKp80, NKG2A, NKG2D, CD335, CD336, CD337, CD94, CD158. Cells events were recorded on a NovoCyte Quanteon Flow Cytometer (Agilent) and analyzed using FlowJo (FlowJo, LLC). FIG. 50A shows that iNK cells derived from edited iPSCs exhibited similar CD56+ surface expression relative to iNK cells derived from unedited iPSC parental clones and PBNK cells (at day 39 in culture). FIG. 50B shows that iNK cells derived from edited iPSCs exhibited similar CD56+ and CD16+ surface expression relative to iNKs derived from unedited iPSC parental clones (at day 39 in culture). FIG. 50C shows that iNK cells derived from edited iPSCs exhibited similar CD56+, CD54+, KIR+, CD16+, CD94+, NKG2A+, NKG2D+, NCR1+, NCR2+, and NCR3+ surface expression relative to iNKs derived from unedited iPSC parental clones and PBNK cells (at day 39 in culture)

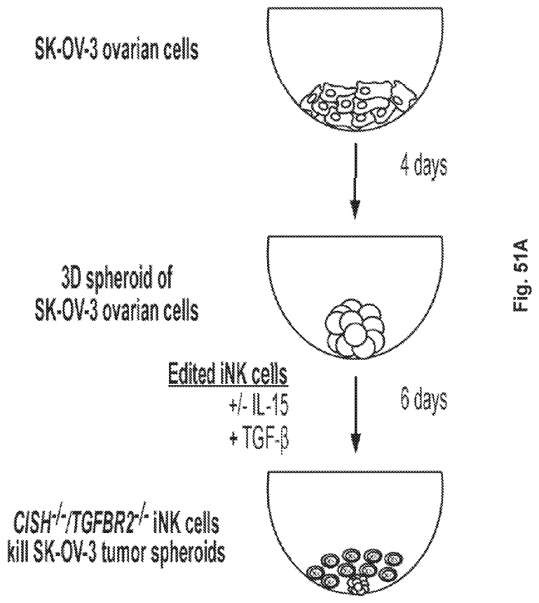

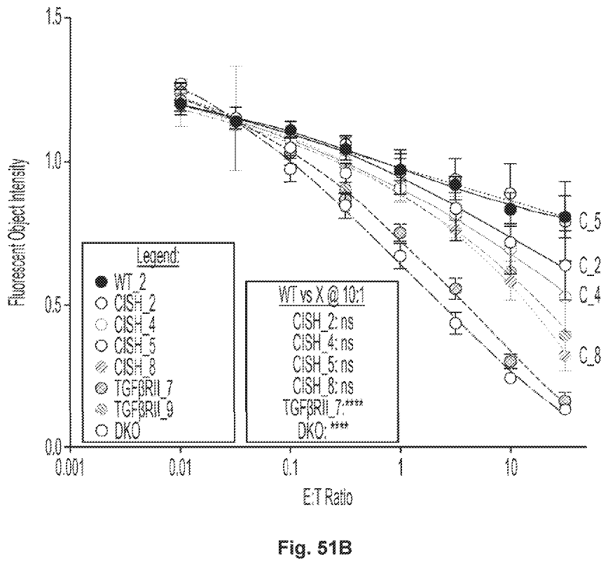

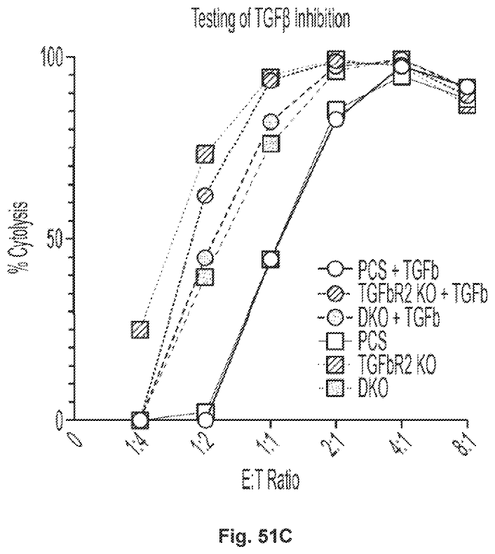

To confirm cell functionality, cells were assessed using a tumor cell cytotoxicity assay on the xCelligence platform. Briefly, tumor targets, SK-OV-3 tumor cells, were plated and grown to an optimal cell density in 96-well xCelligence plates. iNKs were then added to the tumor targets at different E:T ratios (1:4, 1:2, 1:1, 2:1. 4:1 and 8:1) in the presence of TGFβ. FIG. 51C shows that TGFβRII KO and CISH/TGFβRII DKO cells more effectively killed SK-OV-3 cells, as measured by percent cytolysis, relative to unedited iNK cells either in the presence or absence of TGF-β (at E:T ratios of 1:4, 1:2, 1:1, and 2:1).

While iNK cells generated using the alternative method described in FIG. 47B were CD56+ and capable of killing tumor targets in an in vitro cytotoxicity assay, the iNKs did not express many of the canonical markers associated with mature NK cells such as CD16, NKG2A, and KIRs. A K562 feeder cell line is typically used to expand and mature iNKs that are generated by similar differentiation methodologies. After expansion on feeders, the iNKs often express CD16, KIRs and other surface markers indicative of a more mature phenotype. In order to identify a feeder free approach to achieve more mature iNKs with enhanced functionality, an alternative media composition was tested for the stage of differentiation between day 11 and day 39. Instead of culturing cells between day 11 and day 39 in APEL2 (as shown in FIG. 47B), the spin embryoid bodies (SEBs) were cultured in NK MACSR media (MACS Miltenyi Biotec) with 15% human AB serum in the presence of the same cytokines as mentioned above. This protocol is depicted in FIG. 47C. In order to compare the two media compositions, Day 11 SEBs from WT PCS, TGFβRII KO iPSCs, CISH KO iPSCs, and DKO iPSCs were split into two conditions for the second half of the differentiation process, one with APEL2 base and the other with the NKMACS+serum base. At day 39, the cell yield, marker expression, and cytotoxicity levels were assessed. In all cases, the NKMACS+serum condition (depicted in FIG. 47C) outperformed the APEL2 condition (depicted in FIG. 47B). FIG. 47D shows that the NKMACS+serum condition yielded a greater fold expansion at the end of the 39 day process (nearly 300 fold expansion vs 100 fold expansion). When NK marker expression was analyzed by flow cytometry as described above, the iNKs cultured in NKMACS+serum were 34% CD16 positive and exhibited 20% KIR expression while the APEL2 conditions yielded cells that were essentially negative for both markers. This was the case for all genotypes tested. In order to visualize the markers relative to time or condition, flow cytometry data was gated and analyzed in FlowJo and heat maps were constructed (FIGS. 47E and 47F). Samples were first cleaned by gating for live cells (FSC-H vs. LIVE/DEAD™ Fixable Yellow) followed by immune cells (SSC-A vs. FSC-A), singlets (FSC-H vs. FSC-A) and the natural killer cell population (CD56 vs. CD45). The NK population, defined as CD45+56+ cells, was gated and each marker was analyzed along the X-axis in an analysis synonymous to a histogram/count plot (CD16+, CD94+, NKG2A+, NKG2D+, CD335+, CD336+, CD337+, NKp80+, panKIR+). Statistics for the aforementioned markers are visualized with a double-gradient heat map (GraphPad Prism 8) with the key set to the following parameters: black=0), medium intensity 30<x<50), maximum intensity=100. Based on this analysis, the expression kinetics and magnitude across all genotypes were improved by the NKMACS+serum condition. The cells were also assessed in a tumor cell cytotoxicity assay as described previously. The iNKs generated in the NKMACS+serum conditions were capable of killing at a lower E:T ratio than the cells differentiated in APEL2, indicating that the improved NK maturation had a positive impact on the functionality of the cells (FIG. 47G).

Analysis of additional differentiation markers in NKMACS+serum confirmed the presence of CD16 expression. FIG. 50B shows analysis of specific subpopulations (CD45 vs CD56 and CD56 vs CD16) derived from unedited or DKO iPSCs. Additionally, the cell surface marker profile of unedited iNK cells and CISH/TGFβRII DKO iNKs in FIG. 50C confirmed that the NK cell marker profile of the edited iNK cells was similar to that of unedited iNK cells. Taken together, these data show that Cas12a-edited single and double KO iPSC clones differentiate into iNK cells in a similar fashion as unedited iPSC clones, as defined by NK cell markers.

Additionally, certain edited iNK clonal cells (CISH single knockout “CISH_C2, C4, C5, and C8”, TGFβRII single knockout “TGFβRII-C7”, and TGFβRII/CISH double knockout “DKO-C1”), and parental clone iNK cells (“WT”) were cultured in the presence of 1 ng/ml or 10 ng/ml IL-15, and differentiation markers were assessed at day 25, day 32, and day 39 post-hiPSC differentiation. As shown in FIG. 53, surface expression phenotypes (measured as a percentage of the population) culturing in 10 ng/mL IL-15 resulted in a higher proportion of surface expression in the single knockouts, double knockouts, and the parental clonal line.

The edited iNK cells differentiated in NK MACS R: medium+serum conditions were assessed for effector function in vitro using a range of molecular and functional analyses. First, a phosphoflow cytometry assay was performed to determine the phosphorylated state of STAT3 (pSTAT3) and SMAD2/3 (pSMAD2/3) in the day 39 iNK cells, CISH KO iNKs exhibited increased pSTAT3 upon IL-15 stimulation (FIG. 50D), and CISH/TGFβRII DKO iNKs exhibited decreased pSMAD2/3 levels upon TGF-β stimulation as compared to unedited iNK cells (FIG. 50E). These data suggest that CISH/TGFβRII DKO iNKs have enhanced sensitivity to IL-15 and resistance to TGF-β mediated immunosuppression. In addition, CISH/TGFβRII DKO iNKs were characterized for IFNγ and TNFα production using a phorbol myristate acetate and Ionomycin (PMA/IMN) stimulation assay. Briefly, cells were treated with 2 ng/ml of PMA and 0.125 μM of Ionomycin along with a protein transport inhibitor for 4 hours. The cells were harvested and stained using a standard intracellular staining protocol. The CISH/TGFβRII DKO INKs produced significantly higher amounts of IFNγ and TFNα when stimulated with PMA/IMN (FIGS. 50F and 50G), providing evidence of enhanced cytokine production following stimulation relative to unedited control iNKs.

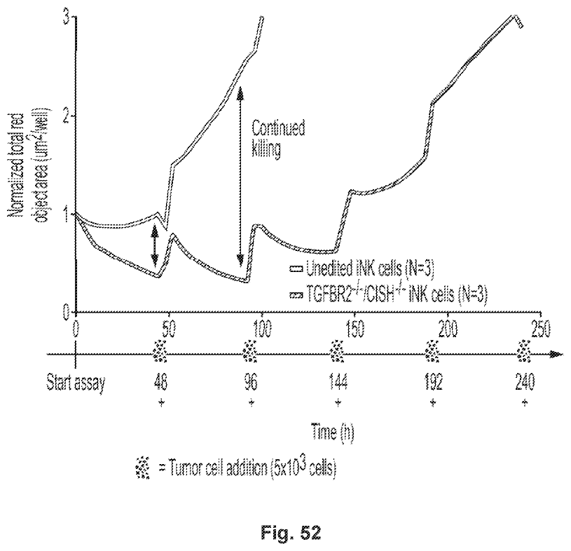

To test iNK tumor cell killing activity, a 3D solid tumor cell killing assay (depicted schematically in FIG. 51A) was utilized. In brief, spheroids were formed by seeding 5,000 NucLight Red labeled SK-OV-3 cells in 96 well ultra-low attachment plates. Spheroids were incubated at 37° ° C. before addition of effector cells (at different E:T ratios) and 10 ng/ml TGF-β, spheroids were subsequently imaged every 2 hours using the Incucyte S3 system for up to 120 hours. Data shown are normalized to the red object intensity at time of effector addition. Normalization of spheroid curves maintains the same efficacy patterns observed in non-normalized data. Using this assay, the cytotoxicity of iNKs differentiated from four CISH KO iPSC clones, two TGFβRII KO iPSC clones and one CISH/TGFβRII DKO iPSC clone were compared to control iNKs derived from the unedited parental iPSCs. As shown in FIG. 51B, edited iNK cells were capable of reducing the size of SK-OV-3 spheroids more effectively than unedited iNK control cells (averaged data from 6 assays). In particular the CISH/TGFβRII DKO iNK cells reduced the size of SK-OV-3 spheroids to a greater extent than unedited iNK cells at all E:T ratios greater than 0.01, and significantly at E:T ratios of 1 or higher. The TGFβRII KO clone 7 iNKs also exhibited significantly enhanced killing when compared to unedited iNK cells. While a number of single CISH KO clones did not show significant enhancement of killing at the 10:1 E:T ratio, the majority of clones did display a trend towards increased SK-OV-3 spheroid cell killing, with the greatest differential at the highest ET ratio. To further elucidate the functionality of the edited iNKs, the cells were pushed to kill tumor targets repeatedly over a multiday period, herein described as an in vitro serial killing assay. At day 0 of the assay, 10×10⁶ Nalm6 tumor cells (a B cell leukemia cell line) and 2×10⁵ iNKs were plated in each well of a 96-well plate in the presence of IL-15 (10 ng/ml) and TGF-β (10 ng/ml). At 48 hour intervals, a bolus of 5×10³ Nalm6 tumor cells (a B cell leukemia cell line) was added to re-challenge the iNK population. As shown in FIG. 52, the edited iNK cells (CISH/TGFβRII DKO INK cells) exhibited continued killing of Nalm6 cells after multiple challenges with Nalm6 tumor cells, whereas unedited iNK cells were limited in their serial killing effect. The data supports the conclusion that the CISH and TGFβRII edits result in prolonged enhancement of cell killing.

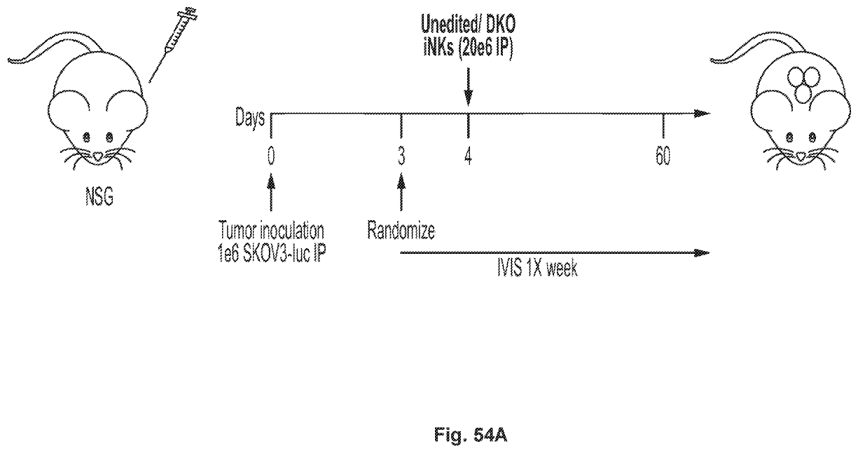

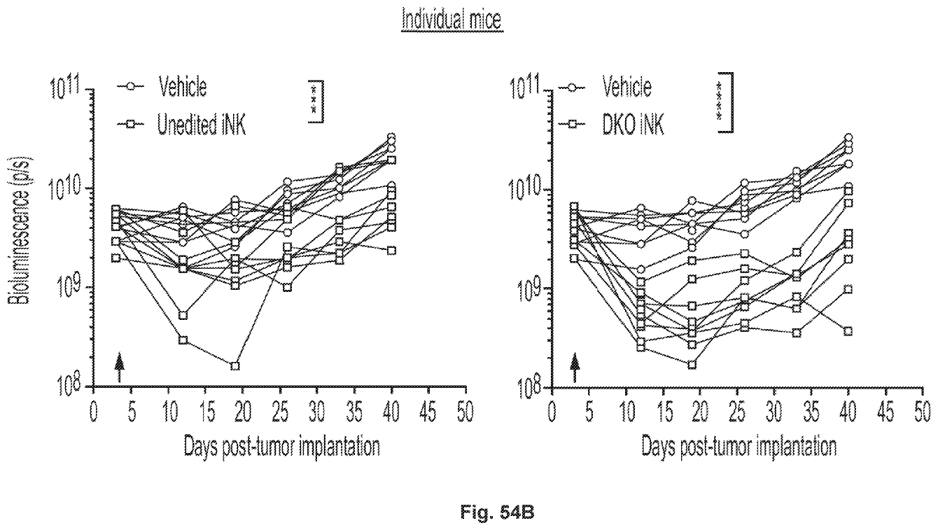

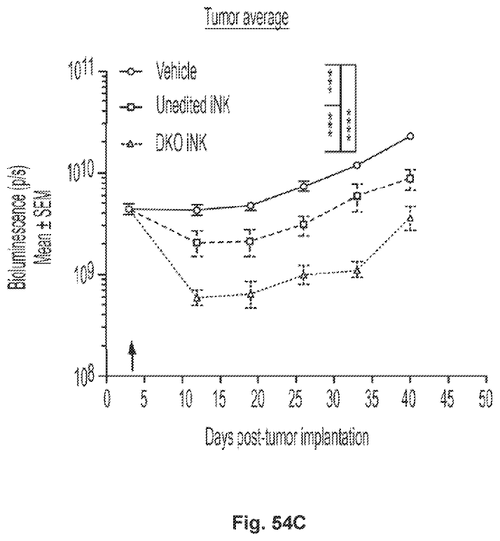

Finally, edited iNK cells (CISH/TGFβRII DKO INK cells) were assayed for their ability to kill tumor targets in an in vivo model. To this end, an established NOD scid gamma (NSG) xenograft model was utilized in an assay as depicted in FIG. 54A. Briefly. 1×10⁶ SK-OV-3 cells engineered to express luciferase were injected intraperitoneally (IP) at day 0). On day 3, the inoculated mice were imaged using an In vivo imaging system (IVIS) and randomized into 3 groups. The next day (day 4), 20×10⁶ unedited iNKs or CISH/TGFβRII DKO iNKs were administered by IP injection, while a third group was injected with vehicle as a control. Following inoculation of the animals with tumor cells, animals were imaged once a week to measure tumor burden over time. FIG. 54B depicts the bioluminescence of the tumors in the individual mice in the 3 different groups (n=9 in each group), vehicle, unedited iNKs, and CISH/TGFβRII DKO iNKs. The average tumor burden over time for these same animals is depicted in FIG. 54C. A two way anova analysis was performed on the data, and CISH/TGFβRII DKO iNK treated animals had significantly less tumor burden as measured by bioluminescence when compared to animals treated with unedited iNKs (p value: 0.0004). By 10 days post-tumor implantation, mice injected with the CISH/TGFβRII DKO iNKs exhibited a significant reduction in the size of their tumors relative to mice injected with the vehicle controls or the unedited iNKs. The overall reduction in tumor size is seen for several days, and at least until 35 days post-tumor implantation. These data show that the edited DKO iNKs were actively killing tumor cells in this in vivo model.

Overall, these results demonstrate that unedited and CISH/TGFβRII DKO iPSCs can be differentiated into iNK cells exhibiting canonical NK cell markers. Additionally, CISH/TGFβRII DKO iNK cells demonstrated enhanced anti-tumor activity against tumor cell lines derived from both solid and hematological malignancies.

### Example 20: ADORA2A Edited iPSCs Give Rise to Edited iNKs with Enhanced Function

ADORA2A is another target gene of interest, the loss of which is hypothesized to affect NK cell function in a tumor microenvironment (TME). The ADORA2A gene encodes a receptor that responds to adenosine in the TME, resulting in the production of cAMP which functions to drive a number of inhibitory effects on NK cells. We hypothesized that knocking out the function of ADORA2A could enhance iNK cell function. Utilizing a similar approach to the one described in Examples 18 and 19, the PCS iPSC line was edited using a RNP having an engineered Cas 12a with three amino acid substitutions (M537R, F870L, and H800A (SEQ ID NO: 62)) and a gRNA specific to ADORA2A (except that 4 μM RNP was delivered to cells rather than 2 μM RNP). As described in Example 18, the gRNA was generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. The ADORA2A gRNA sequence is shown in Table 27.

TABLE 27
Guide RNA sequence

| | gRNA | |
|---|---|---|
| | Targeting | |
| | Domain | |
| Target | Sequence | Full Length gRNA Sequence |
| ADORA2A | CCAUCGGC | ATGTGTTTTTGTCAAAAGACCTTTTrUrA |
| 4113 | CUGACUCC | rArUrUrUrCrUrArCrUrCrUrUrGrUr |
| | CAUG | ArGrArUrCrCrArUrCrGrGrCrCrUrG |
| | (SEQ ID | rArCrUrCrCrCrArUrG (SEQ ID |
| | NO: 1159) | NO: 1160) |

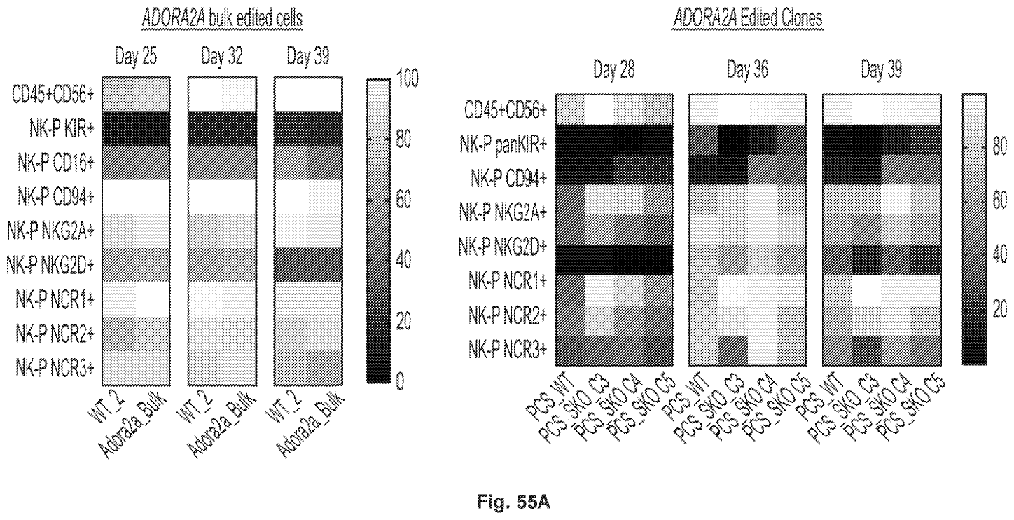

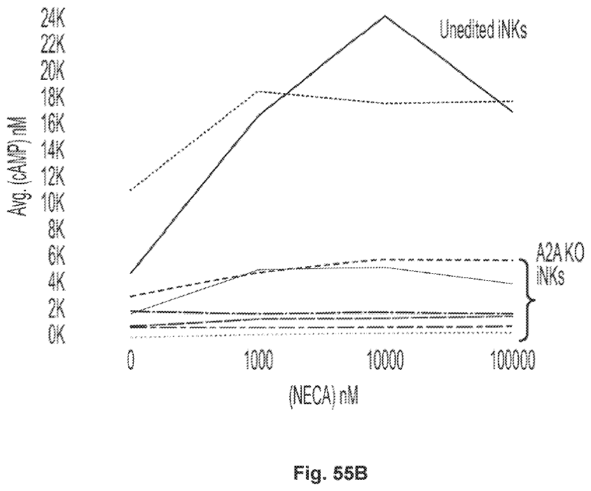

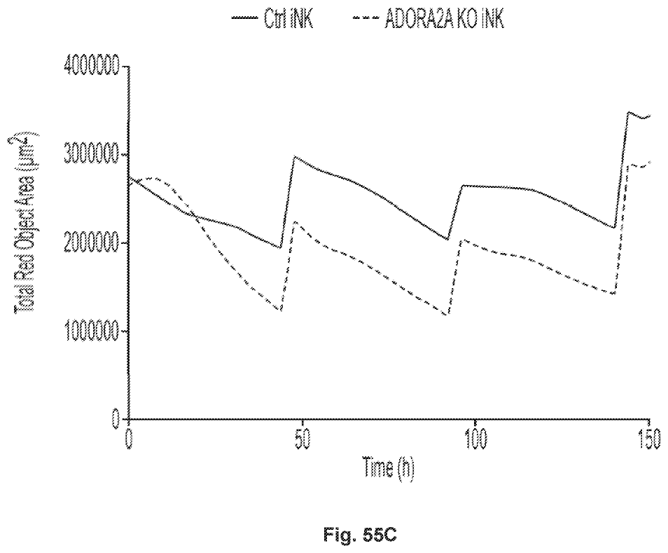

The bulk editing rate by the Cas12a RNP prior to clonal selection was 49% as determined by next-generation sequencing (NGS). Nonetheless, several clones that had both ADORA2A alleles edited were identified, expanded and differentiated. To determine whether an ADORA2A edited iPSC could yield CD45+CD56+ iNKs, both bulk and singled ADORA2A KO clones were differentiated using the NKMACS+serum protocol as described in Example 19 (FIG. 47C). As shown in FIG. 55A, edited iPSCs differentiated to iNKs with similar NK cell marker expression compared to unedited control iPSCs.

To confirm that Cas 12a-mediated ADORA2A editing resulted in a functional deletion of the gene, cAMP accumulation in response to treatment with 5′-N-ethylcarboxamide adenosine (“NECA”, a more stable adenosine analog that acts as an ADORA2A agonist) was assessed in both the edited and unedited control iNKs. Edited cells with a functional knockout of ADORA2A would not be expected to accumulate as much CAMP in the cells in response to NECA relative to cells with functional ADORA2A. Briefly, iNK cells were treated with varying concentrations of NECA for 15 minutes. The iNK cells were then lysed, and the CAMP in the lysate was then measured using a CisBio CAMP kit. As shown in FIG. 55B, unedited iNKs had increased levels of cAMP accumulation as the concentration of NECA was increased (n=2). Conversely, the ADORA2A (“A2A KOs”) KO iNKs showed minimal production of cAMP at increasing concentrations of NECA, indicating that the Cas12a-induced edits functionally knocked out ADORA2A function. The bulk iNKs (top two A2A KO iNK lines in FIG. 55B) exhibited slightly higher levels of cAMP than the selected ADORA2A KO clones (lower four A2A KO iNK lines in FIG. 55B), as would be expected from the lower editing rates in the bulk population. Based on this molecular evidence of functional ablation of ADORA2A, the iNKs would be expected to be resistant to the inhibitory effects of adenosine in a tumor microenvironment.

The ADORA2A KO iNKs were also tested in an in vitro NALM6 serial killing assay as described in Example 19, with one main difference: 100 μM of NECA was added in place of TGFβ. The ADORA2A KO iNKs exhibited enhanced serial killing relative to the wild type iNKs in the presence of NECA, indicating that the ADORA2A KO iNKs were resistant to NECA inhibition (FIG. 55C). As a result, the ADORA2A KO iNK cells would be expected to have improved cytotoxicity against tumor cells in the presence of adenosine in the TME relative to unedited iNK cells.

### Example 21: Generation of CISH/TGFβRII/ADORA2A Triple Edited (TKO) iPSCs and the Characterization of Differentiated TKO iNKs

In order to generate CISH, TGFβRII, and ADORA2A triple edited (TKO) iPSCs, two approaches were taken: 1) two step editing in which the CISH/TGFβRII DKO (CR) iPSC clone described in Examples 18 and 19 was edited at the ADORA2A locus via electroporation with an ADORA2A targeting RNP (as described in Example 20), and 2) simultaneous editing of PCS iPS cells with all 3 RNPs, one for each target gene. Both strategies utilized the editing protocol briefly described in Example 18. In the case of simultaneous editing, the total RNP concentration was 8 μM (Cish:2 μM+TGFβRII:2 μM+ADORA2A:4 μM). Regardless of the approach, cells were plated, expanded and colonies were picked as described above. Using NGS to analyze gDNA harvested from the iPSCs, it was determined that the bulk editing rates were 96.70%, 97.17%, and 90.16% for CISH, TGFβRII and ADORA2A, respectively, when all target genes were edited simultaneously. Picked colonies that had Insertions and/or Deletions (InDels) at all 6 alleles were selected for further analysis.

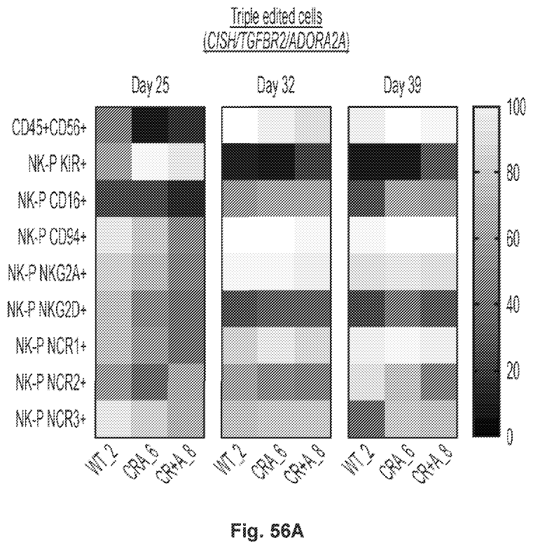

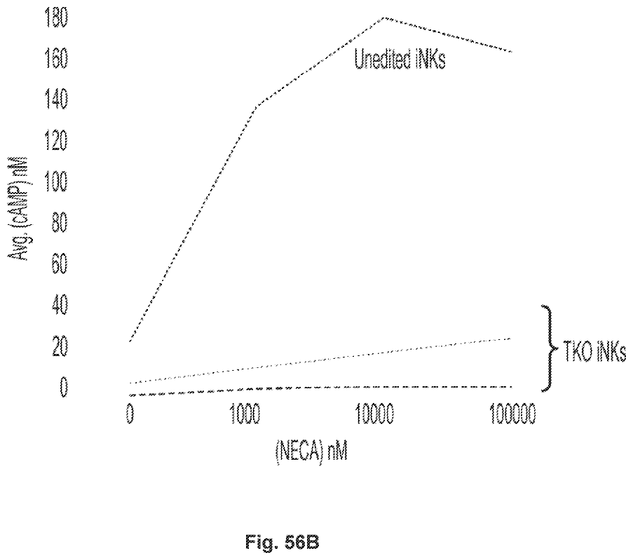

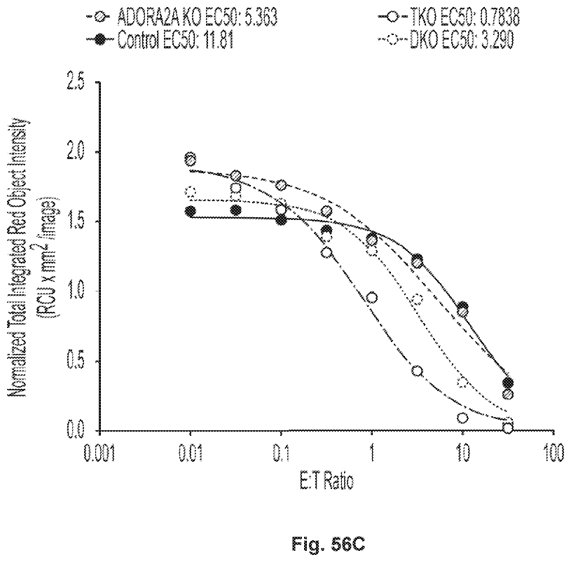

Similar to the analysis described in Example 18, unedited iPSCs and the edited iPSCs were differentiated to iNKs using the NK MACS+Serum condition (described in FIG. 49C) and assessed by flow cytometry at different time points, including at day 25, day 32, and day 39 in culture. As shown in FIG. 56A, analysis of the different NK surface markers revealed no major differences between clones that were generated by the two-step editing method (CR+A 8) or the simultaneous editing method (CRA 6). Both TKO clones (CR+A 8 and CRA 6) showed similar expression profiles to the unedited iNKs (Wt) at each time point. When the TKO iNK cells were analyzed for their responsiveness to NECA (as described in Example 20), both TKO iNKs had little to no cAMP accumulation (FIG. 56B), demonstrating that ADORA2A was functionally knocked out. By contrast, the unedited iNKs demonstrated a NECA dose dependent increase in cAMP (FIG. 56B). These results indicate that the TKO iNKs would be expected to be resistant to the inhibitory effects of adenosine in the TME. Finally, the CISH/TGFβRII/ADORA2A TKO iNKs were assessed alongside CISH/TGFβRII DKO INKs, ADORA2A single KO (SKO) iNKs, and unedited iNKs in a 3D tumor cell killing assay. This assay was performed as described in Example 19 with IL-15 and TGFβ but without NECA. Interestingly, both the TKO (CRA6) and DKO (CR) iNKs outperformed the unedited iNKs in killing the tumor cells, indicating that both multiplex edited iNKs have enhanced function over unedited control cells (FIG. 56C). These results show that knocking out ADORA2A does not negatively affect the ability of iNKs having CISH and TGFβRII KOs to kill tumor spheroid cells.

### Example 22: Selection of CISH, TGFβRII, ADORA2A, TIGIT, and NKG2A Targeting gRNAs

The cutting efficiency of CISH, TGFβRII, ADORA2A, TIGIT, and NKG2A Cas 12a guide RNAs were further tested. Guide RNAs were screened by complexing commercially synthesized gRNAs with Cas 12a in vitro and delivering gRNA/Cas12a ribonucleoprotein (RNP) to IPSCs via electroporation. The iPSCs were edited using a RNP having an engineered Cas12a with three amino acid substitutions (M537R, F870L, and H800A (SEQ ID NO: 62)). The gRNAs were generated with a targeting domain consisting of RNA, an AsCpf1 scaffold of the sequence UAAUUUCUACUCUUGUAGAU (SEQ ID NO: 1153) located 5′ of the targeting domain, and a 25-mer DNA extension of the sequence ATGTGTTTTTGTCAAAAGACCTTTT (SEQ ID NO: 1154) at the 5′ terminus of the scaffold sequence. Table 28 provides the targeting domains of the guide RNAs that were tested for editing activity.

TABLE 28
guide RNA sequences

| | gRNA Targeting |
|---|---|
| Target | Domain Sequence |
| TGFβRII | UGAUGUGAGAUUUUCCACCUG |
| | (SEQ ID NO: 1161) |
| CISH | ACUGACAGCGUGAACAGGUAG |
| | (SEQ ID NO: 1162) |
| ADORA2A | CCAUCGGCCUGACUCCCAUGC |
| | (SEQ ID NO: 1163) |
| ADORA2A | CCAUCACCAUCAGCACCGGGU |
| | (SEQ ID NO: 1164) |
| ADORA2A | CCUGUGUGCUGGUGCCCCUGC |
| | (SEQ ID NO: 1165) |
| TIGIT | UGCAGAGAAAGGUGGCUCUAU |
| | (SEQ ID NO: 1166) |
| TIGIT | UCUGCAGAAAUGUUCCCCGUU |
| | (SEQ ID NO: 1167) |
| TIGIT | UAGGACCUCCAGGAAGAUUCU |
| | (SEQ ID NO: 1168) |
| NKG2A | GCAACUGAACAGGAAAUAACC |
| | (SEQ ID NO: 1169) |
| NKG2A | GUUGCUGCCUCUUUGGGUUUG |
| | (SEQ ID NO: 1170) |
| NKG2A | AAGGGAAUGACAAAACCUAUC |
| | (SEQ ID NO: 1171) |

In brief, 100,000 iPSCs/well were transfected with the RNP of interest, cells were incubated at 37ºC for 72 hours, and then harvested for DNA characterization. iPSCs were transfected with gRNA/Cas12a RNPs at various concentrations. The percentage editing events were determined for eight different RNP concentrations ranging from negative control (0 mM) to 8 mM.

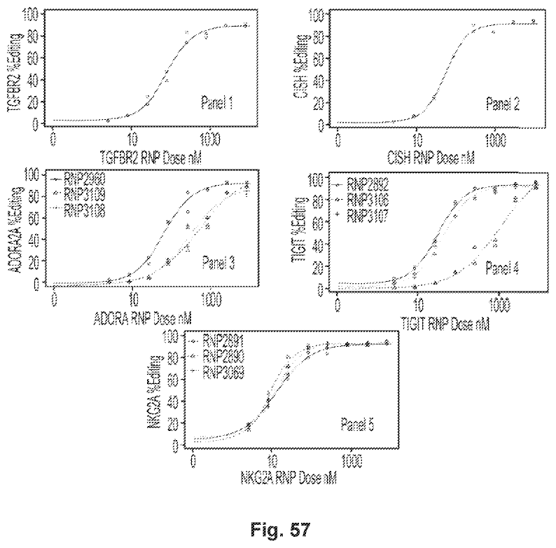

As shown in FIG. 57 panel 1, the TGFβRII gRNA (SEQ ID NO: 1161) exhibited an EC50 of ˜79 nM RNP. As shown in FIG. 57 panel 2, the CISH gRNA (SEQ ID NO: 1162) exhibited an EC50 of ˜50 nM RNP. As shown in FIG. 57 panel 3, an ADORA2A gRNA (SEQ ID NO: 1163) included in RNP2960 exhibited an EC50 of ˜63 nM RNP, while an ADORA2A gRNA (SEQ ID NO: 1164) included in RNP3109, or gRNA (SEQ ID NO: 1165) included in RNP3108 exhibited EC50 values of ˜493 nM and ˜280 nM RNP respectively. As shown in FIG. 57 panel 4, a TIGIT gRNA (SEQ ID NO: 1166) included in RNP2892 exhibited an EC50 of ˜29 nM RNP, while a TIGIT gRNA (SEQ ID NO: 1167) included in RNP3106, or gRNA (SEQ ID NO: 167) included in RNP3107 exhibited EC50 values of ˜1146 nM and ˜40 nM RNP respectively. As shown in FIG. 57 panel 5, a NKG2A gRNA (SEQ ID NO: 1169) included in RNP19142 exhibited an EC50 of ˜8 nM RNP, while a NKG2A gRNA (SEQ ID NO: 1170) included in RNP3069, or gRNA (SEQ ID NO: 1171) included in RNP2891 exhibited EC50 values of ˜12 nM and ˜13 nM RNP respectively.

### Example 23: Knock-In of Cargo at Essential Gene Loci in T-Cells Using a Viral Vector

The present example describes gene editing of populations of T cells using viral vector transduction. Following editing, cells were subjected to various assays such as flow cytometry, ddPCR, next-generation sequencing, or functional tumor killing assays to determine KO/KI efficiency and/or efficacy.

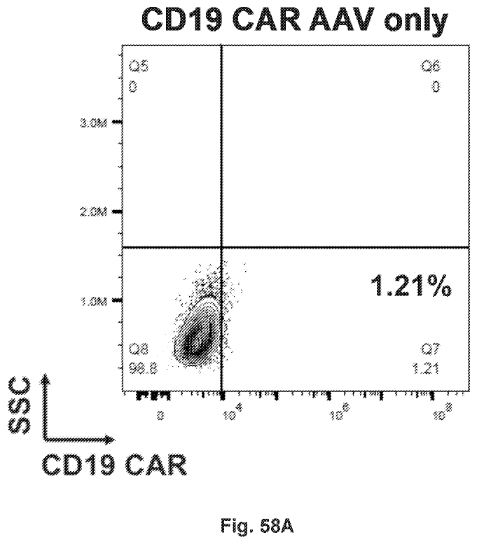

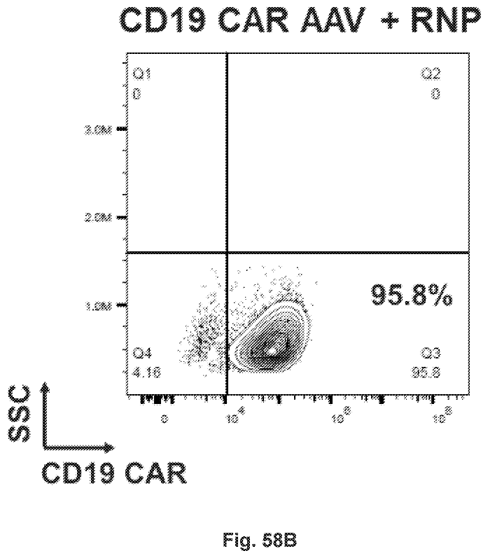

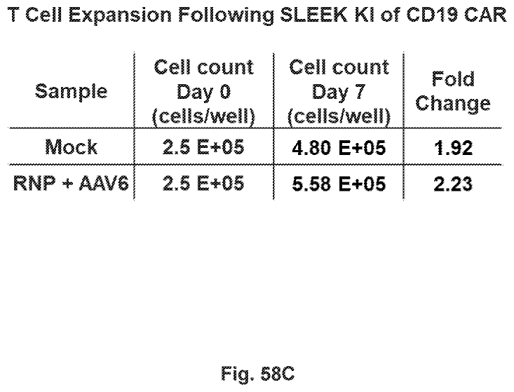

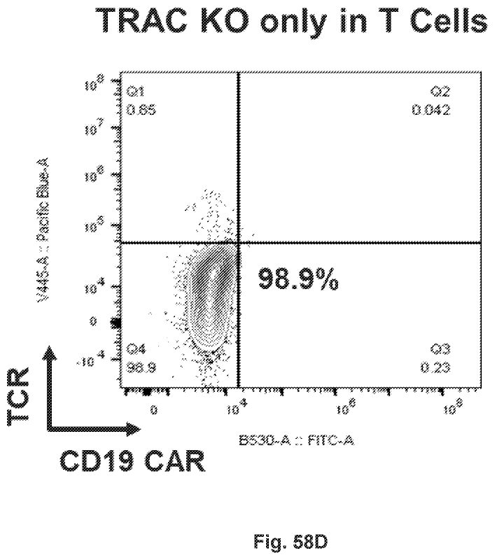

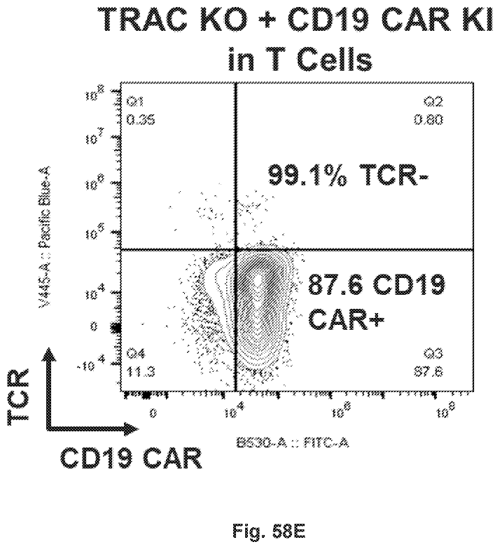

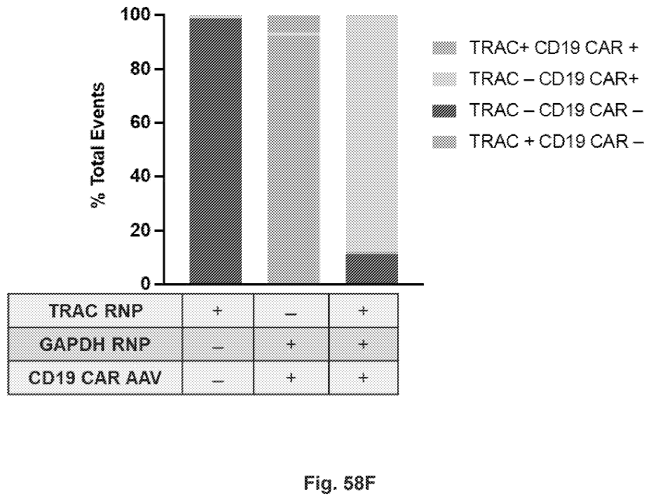

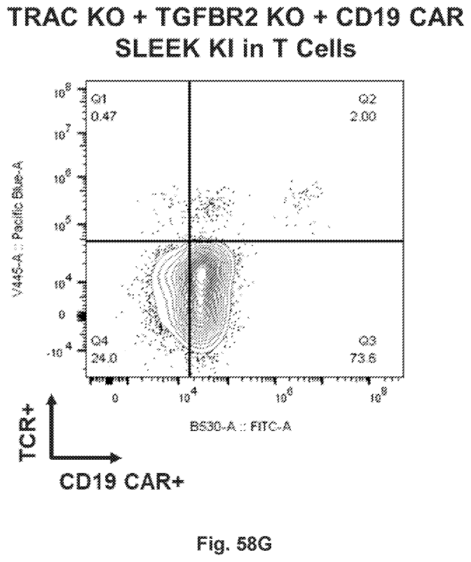

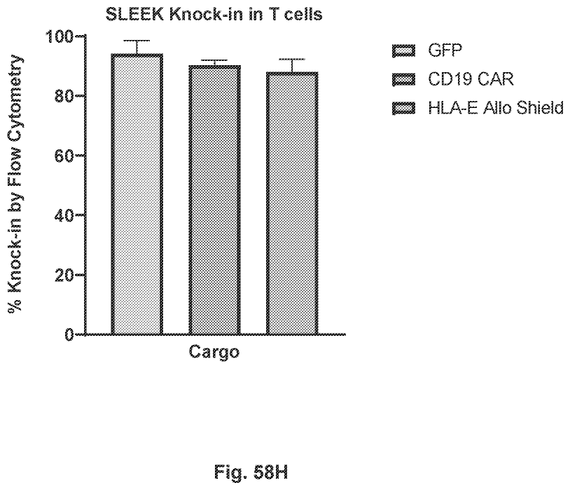

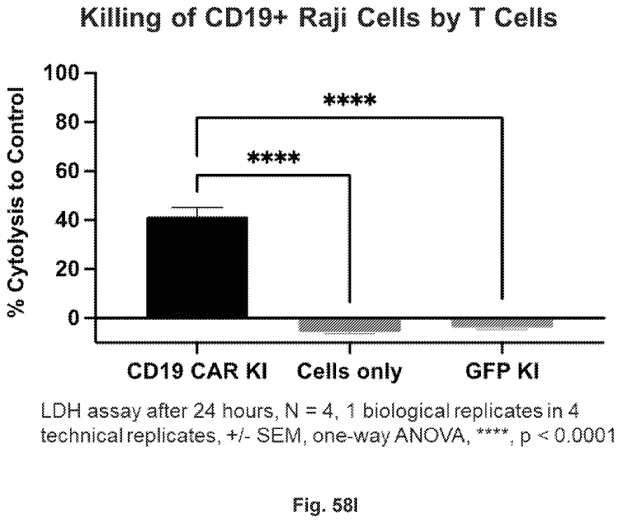

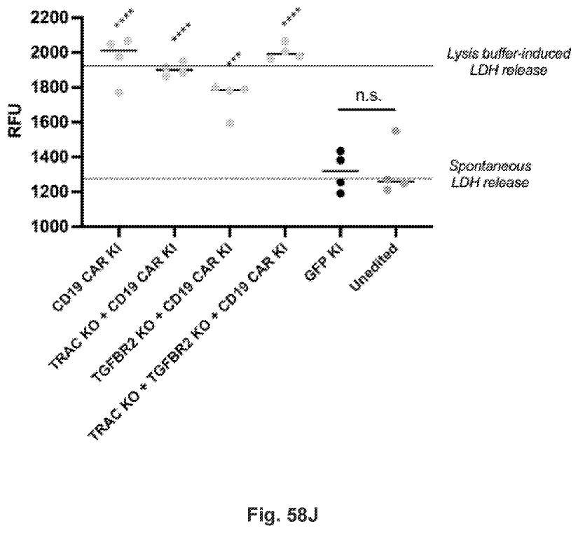

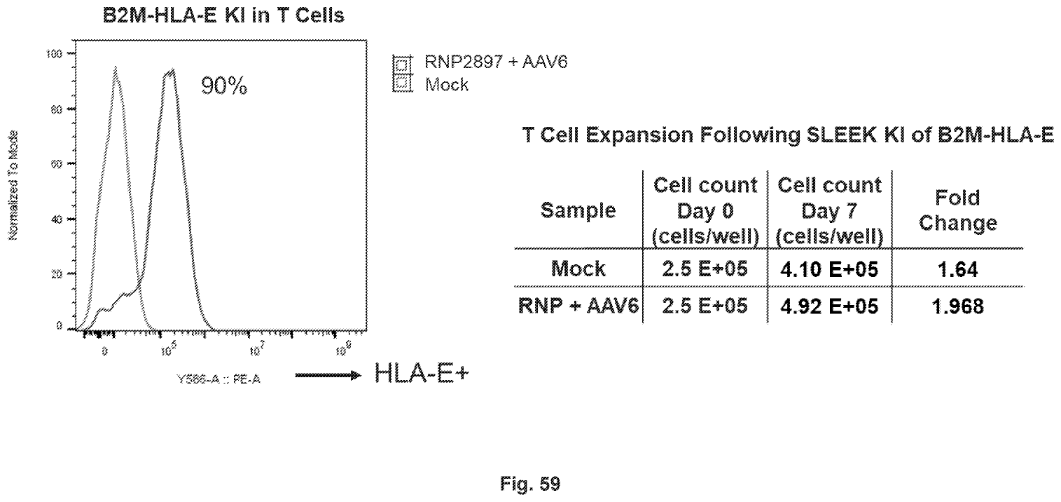

T cells were thawed in a bead bath as known in the art and were removed from the bath on day two. Cells were electroporated on day four after thawing. Briefly, 250,000 T cells per well in a Lonza 96-well cuvette were suspended in buffer P2 and electroporated with RNP comprising gRNA RSQ22337 (SEQ ID NO: 95) and Cas 12a (SEQ ID NO: 62) targeting the GAPDH gene (1 μM RNP) or with media control, using various pulse codes. Appropriate media was added to cells immediately after electroporation and cells were allowed to recover for 15 minutes. AAV6 viral particles comprising a donor plasmid construct containing a knock-in cassette with a cargo of GFP, CD19 CAR, B2M-HLA-E, or vector control were then added to T cells at varying multiplicity of infection (MOI) concentrations (1E4, 1E5, or 1 E6 MOI (vg/cell)). The donor plasmids were designed as described in Example 2, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for a cargo sequence (e.g., GFP, CD19 CAR, or B2M-HLA-E) (“Cargo”), a stop codon and polyA signal sequence. T cells were split two days later, and then every 48 hours until they were analyzed by flow cytometry or otherwise utilized. T cells were sorted using flow cytometry seven days post electroporation to determine successful transduction, transformation, editing, knock-in cassette integration, and/or expression events. A very high percentage (94.8%) of cells expressed GFP, indicating that a high proportion of cells had GFP integrated at the GAPDH gene in the edited T cell population, and these edited cells exhibited similar viability and ability to expand as control cells that underwent mock transformation (FIG. 17B-17C). Moreover, GFP knock-in at the GAPDH locus generated GFP+ cells at a significantly greater rate than GFP knock-in at the TRAC locus (FIG. 17D). This increase in GFP+ cells produced by GFP knock-in at the GAPDH locus compared to GFP knock-in at the TRAC locus was observed across a range of AAV6 concentrations (FIG. 17E). These results demonstrate that knock-in at an essential gene locus (e.g., GAPDH) can achieve greater knock-in efficiency, including at lower concentrations of the AAV6 donor template, than knock-in at the TRAC locus. A very high percentage (95.8%) of cells expressed CD19 CAR, indicating that a high proportion of cells had CD19 CAR integrated at the GAPDH gene in the edited T cell population, and these edited cells also exhibited similar viability and ability to expand as control cells that underwent mock transformation (FIGS. 58A-58C, and 58H). Additionally, a very high percentage (over 80%) of cells expressed B2M-HLA-E, indicating that a high proportion of cells had B2M-HLA-E integrated at the GAPDH gene in the edited T cell population. As shown in FIG. 58H, T cells that were edited to include knock-in of GFP, CD19 CAR, or B2M-HLA-E expressed these transgenes at rates that were greater than 80%. Furthermore, B2M-HLA-E KI cells expressed a higher level of HLA-E when compared to control cells and were viable (see FIG. 59).

The knock-in efficiencies generated using the methods described herein were compared to the knock-in efficiencies generated using optimized methods known in the art for targeting cargo knock-ins to non-essential genes. In brief, T cell populations were transduced with AAV6 vector comprising a donor template suitable for knock-in of GFP at the GAPDH gene as described herein, and were transformed with gRNA RSQ22337 (SEQ ID NO: 95) and Cas 12a (SEQ ID NO: 62) as described above. Alternatively, T cell populations were subjected to highly optimized GFP knock-in at the TRAC locus using AAV6 vector transduction (see e.g., Vakulskas et al. A high-fidelity Cas9 mutant delivered as a ribonucleoprotein complex enables efficient gene editing in human hematopoietic stem and progenitor cells. Nat Med. 2018:24(8): 1216-1224). Flow cytometry was utilized to measure knock-in efficiency (determined by percentage of T cell population expressing GFP, measured 7 days post-electroporation). Knock-in rates at the TRAC locus were high (˜50%) when compared to publicly described integration frequencies for similar methodologies: however, knock-in efficiency at the GAPDH gene using the methods described herein were significantly (p=<0.001 using unpaired t-test) higher (˜90%) (see FIG. 17D). The same RNP concentration, AAV6 MOI, and homology arm lengths were utilized in both experiments, averaged results from three independent replicates are shown. Thus, the methods described herein can be used to isolate a population of modified cells, such as immune cells like T cells, that more highly express a gene of interest relative to other gene knock-in methods.

In other experiments, T cells were edited to generate TRAC knock-out cells without (see FIG. 58D) or with (see FIG. 58E) a CD19 CAR KI at the GAPDH locus using the methods described above. As shown in FIG. 58E, a very high percentage of edited cells expressed CD19 CAR (87.6%) indicating high levels of CD19 CAR integration at the GAPDH gene. By contrast, the control TRAC KO cells did not express CD19 CAR (FIG. 58D). As shown in FIG. 58F. T cells transformed with TRAC targeting RNPs, GAPDH targeting RNPs, and/or transduced with AAV6 comprising a CD19 cargo targeted for knock-in at GAPDH displayed various phenotypes representative of their respective desired edited genotypes. As determined by flow cytometry. T cells that had CD19 CAR KI were observed at rates greater than 90% when cells were transformed with GAPDH targeting RNPs, and transduced with AAV6 comprising a knock-in CD19 cargo targeting GAPDH. T cells that had TRAC KO and CD19 CAR KI were observed at rates greater than 80% when cells were transformed with TRAC targeting RNPs, GAPDH targeting RNPs, and transduced with AAV6 comprising a CD19 cargo targeted for knock-in at GAPDH. As depicted in FIG. 58I, T cells with CD19 CAR KI at GAPDH were able to destroy hematological cancer cells (CD19+Raji cells) at rates significantly greater than T cells with GFP KI at GAPDH (“Cells only” refers to unedited T cells). Furthermore, T cells with CD19 CAR KI at GAPDH demonstrated significantly greater cytotoxicity against Raji cells than either T cells with GFP KI at GAPDH or unedited T cells as seen in FIG. 58J. This significant increase in cytotoxicity was also observed with T cells with CD19 CAR KI at GAPDH in combination with TRAC and/or TGFBR2 KO (FIG. 58J).

In other experiments, a population of T cells were edited to generate KO of TRAC, KO of TGFBR2, and CD19 CAR KI at the GAPDH locus using the methods described above, thereby generating triple mutant (TRAC KO, TGFBR2 KO, and CD19 CAR KI) T cells at a high efficiency. A high percentage of the edited T cells (about 73.6%) expressed CD19 CAR (see FIG. 58G).

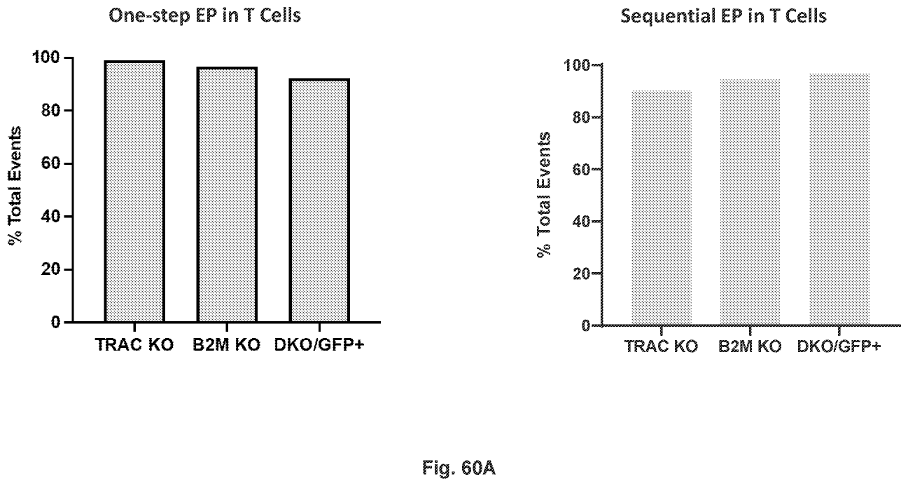

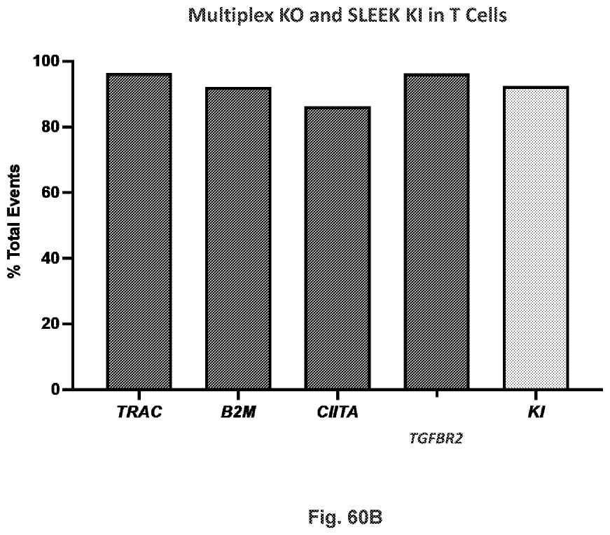

As shown in FIG. 60A, highly defined engineered T cells comprising multiple edits can be generated using a one-step electroporation and transformation process in which three RNPs targeting three loci (TRAC, B2M and GAPDH) and an AAV comprising a GFP cargo for knock-in at the GAPDH locus are applied to the T cells (FIG. 60A left panel), or using a sequential electroporation and transformation process in which the same RNPs and AAVs are sequentially applied to the T cells (FIG. 60A right panel). The one-step process generated about the same percentage of cells containing TRAC and B2M knockouts and GFP expression as the sequential process. In addition. T cells were edited to generate multiple knock-outs including at the TRAC locus, B2M locus, CIITA locus, and TGFBR2 locus as well as a GFP cargo knock-in at the GAPDH locus using a one-step process wherein five Cas 12a (SEQ ID NO:62) RNPs (specific to TRAC, B2M, CIITA, TGFBR2, and GAPDH) and an AAV6 comprising a GFP cargo designed to integrate within the GAPDH locus were applied to the cells at once (see FIG. 60B). Each individual editing event occurred within greater than 80% of the total population, while cells that at least comprised three mutations (TRAC KO, B2M KO, and GFP cargo KI at the GAPDH locus) occurred at a rate greater than 80%.

These results show that the methods described herein can produce highly engineered T cell populations with high levels of editing homogeneity for potential use as autologous and/or allogeneic T cell therapies suitable for targeting a variety of tumors and/or cancerous cells.

### Example 24: Knock-In of Cargo at Essential Gene Loci in NK Cells Using a Viral Vector

The present example describes gene editing of populations of NK cells using viral vector transduction.

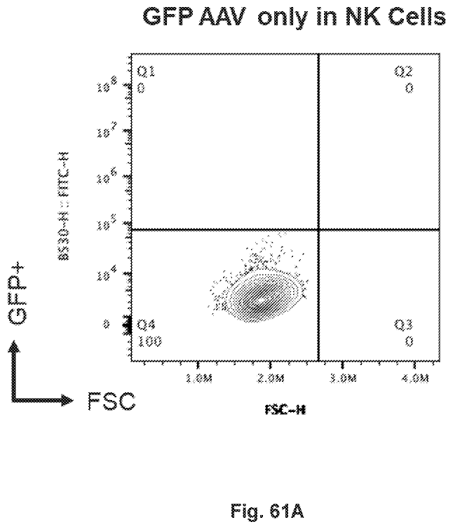

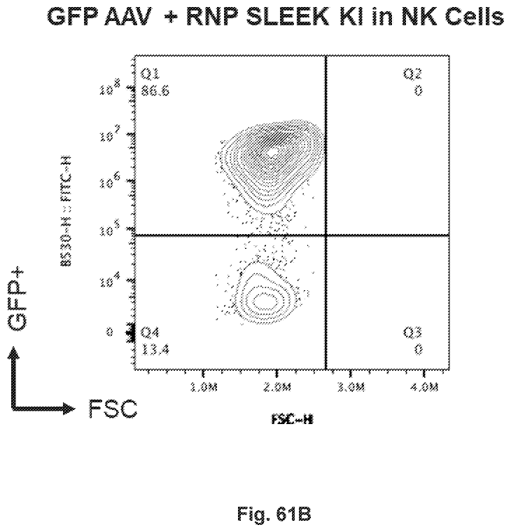

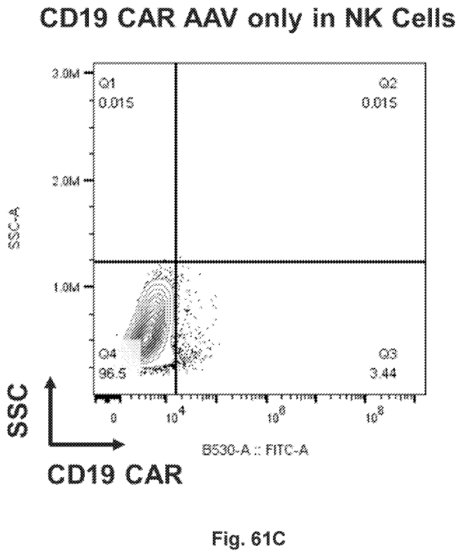

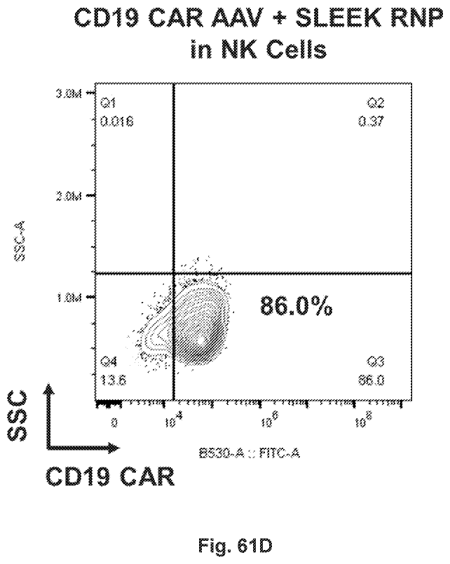

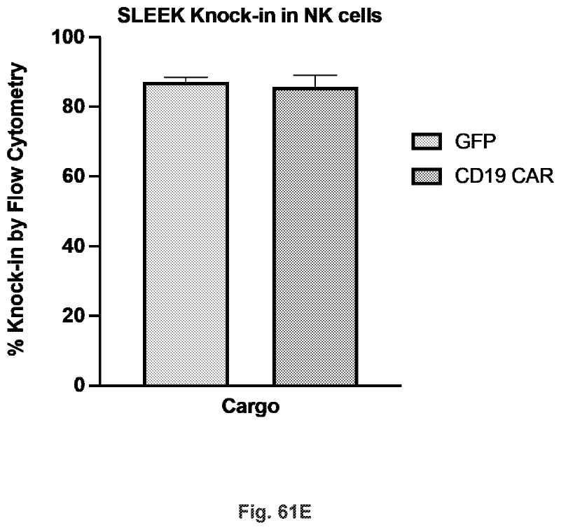

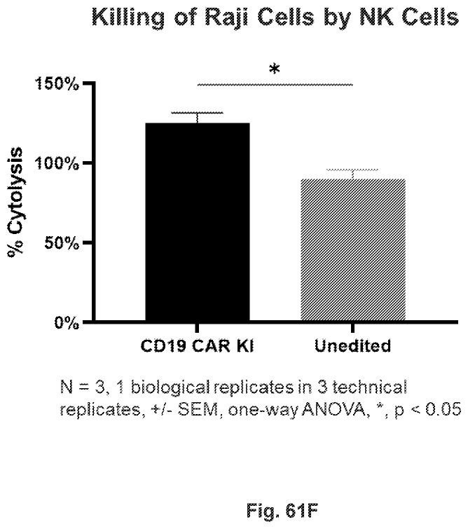

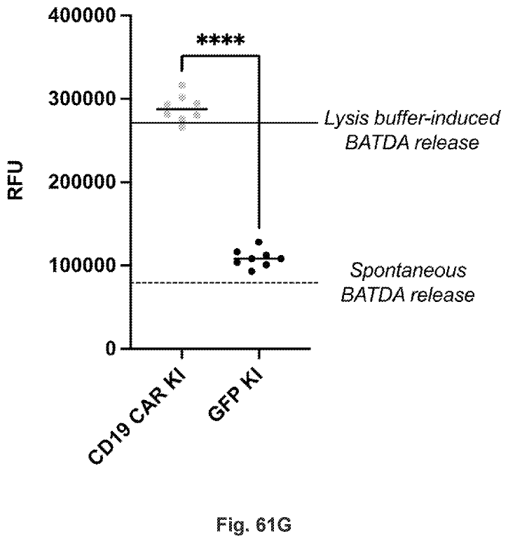

NK cells were thawed in a bead bath as known in the art and were removed from the bath on day two. Cells were electroporated on day four after thawing. Briefly, 500,000 NK cells per well in a Lonza 96-well cuvette were suspended in buffer P2 and electroporated with RNP comprising gRNA RSQ22337 (SEQ ID NO: 95) and Cas 12a (SEQ ID NO: 62) targeting the GAPDH gene (1 μM RNP) or media control, using various pulse codes. Appropriate media was added to cells immediately after electroporation and cells were allowed to recover for 15 minutes. AAV6 viral particles comprising a donor plasmid construct containing a knock-in cassette with a cargo of GFP, CD19 CAR, or vector control were then added to NK cells at varying multiplicity of infection (MOI) concentrations (1E4, 1E5, or 1E6 MOI (vg/cell)). The donor plasmids were designed as described in Example 2, with a 5′ codon-optimized coding portion of GAPDH exon 9 optimized to prevent further binding of the gRNA targeting domain sequence of the guide RNA (RSQ22337)), an in-frame sequence encoding the P2A self-cleaving peptide (“P2A”), an in-frame coding sequence for a cargo sequence (e.g., GFP, or CD19 CAR) (“Cargo”), a stop codon and polyA signal sequence. Media was changed 24 hours post electroporation and IL15 was added. Media was changed again at 72 hours post electroporation, cells were split and 10 ng/ml IL15 was added. NK cells were then split every 48 hours until they were analyzed by flow cytometry or otherwise utilized. NK cells were sorted using flow cytometry seven days post electroporation to determine successful transduction, transformation, editing, knock-in cassette integration, and/or expression events. A very high percentage of cells expressed GFP (86.6%), indicating that a high proportion of edited cells had GFP integrated at the GAPDH gene in the edited NK cell population when compared to a population of control NK cell population that was not transfected with an RNP targeting GAPDH (FIGS. 61A and 61B). Additionally, a very high percentage of cells expressed CD19 CAR, indicating that a high proportion of edited cells had CD19 CAR integrated at the GAPDH gene in the edited NK cell population when compared to a control NK cell population that was not transfected with an RNP targeting GAPDH (FIG. 61C-61D). The methods described herein produced populations of edited NK cells with knock-in of GFP or CD19 CAR at rates greater than 80% (see FIG. 61E). As depicted in FIG. 61F. NK cells with CD19 CAR KI at GAPDH were able to effectively destroy Raji cells at rates significantly greater than unedited NK cells. Furthermore. NK cells with CD19 CAR KI at GAPDH also demonstrated significantly greater cytotoxicity against Nalm6 cells than NK cells with GFP KI at GAPDH (FIG. 61G). These results show that the methods described herein can produce engineered NK cell populations with high levels of editing homogeneity for potential use as autologous and/or allogeneic NK cell therapies suitable for targeting a variety of tumors and/or cancerous cells.

### Example 25: Knock-Out of CISH and TGFβRII in Combination with Knock-In of Bicistronic CD16 and mbIL-15 Sequences at an Essential Gene Locus

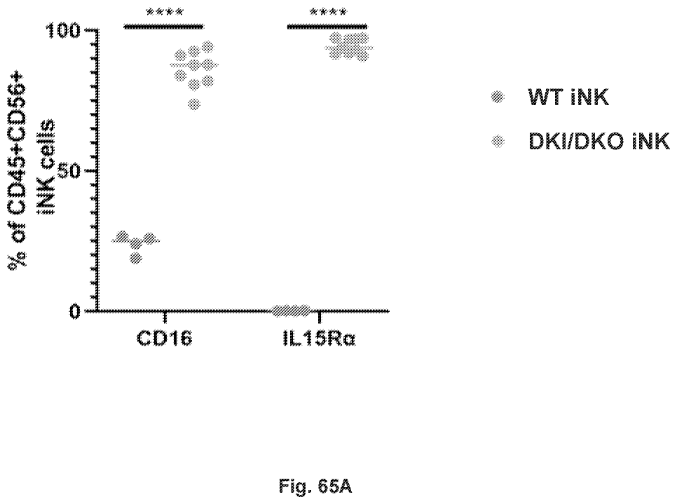

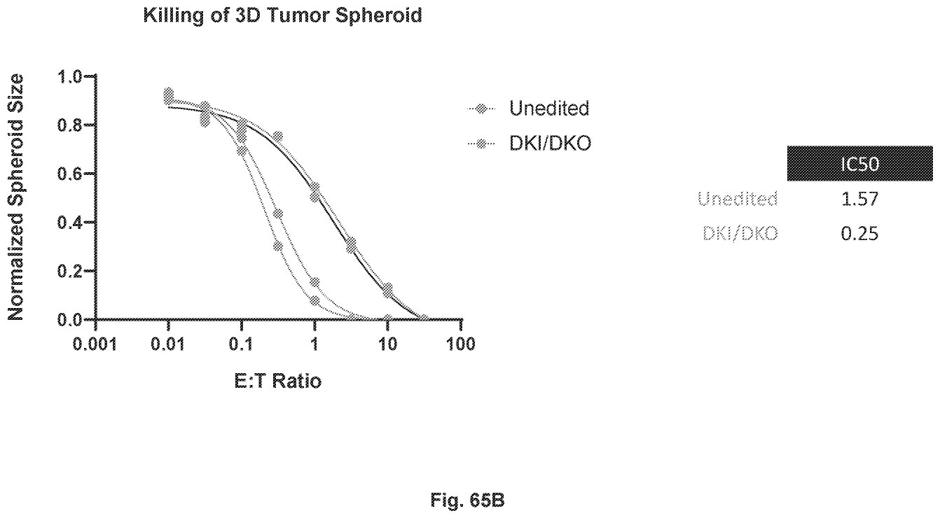

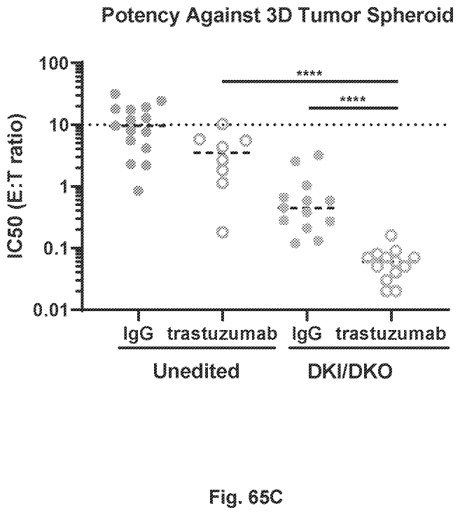

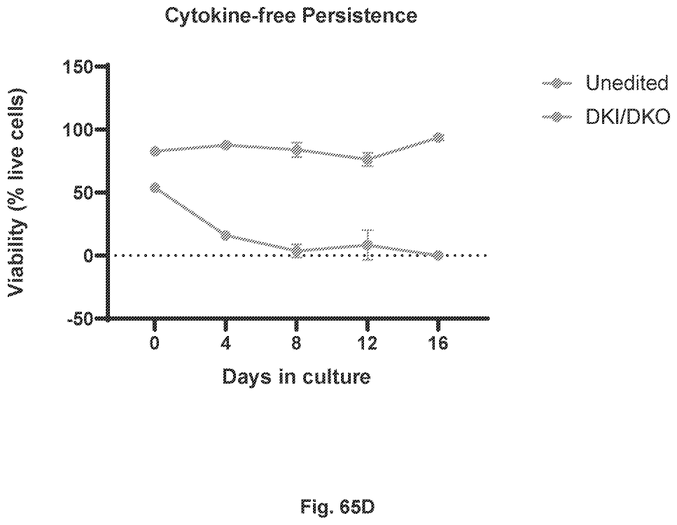

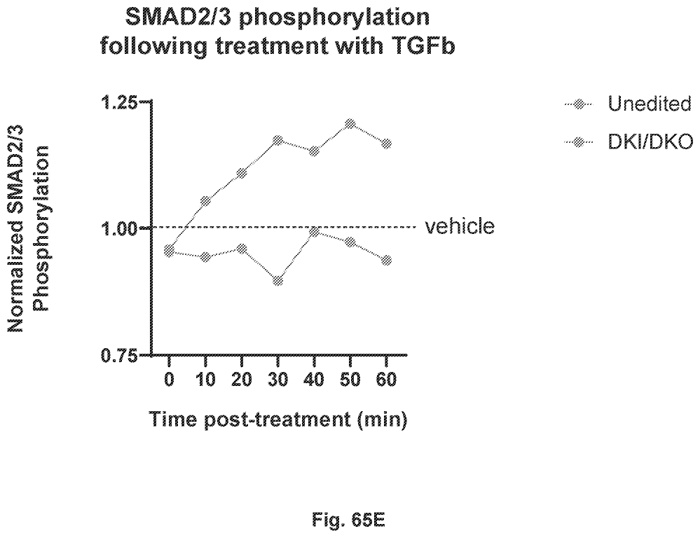

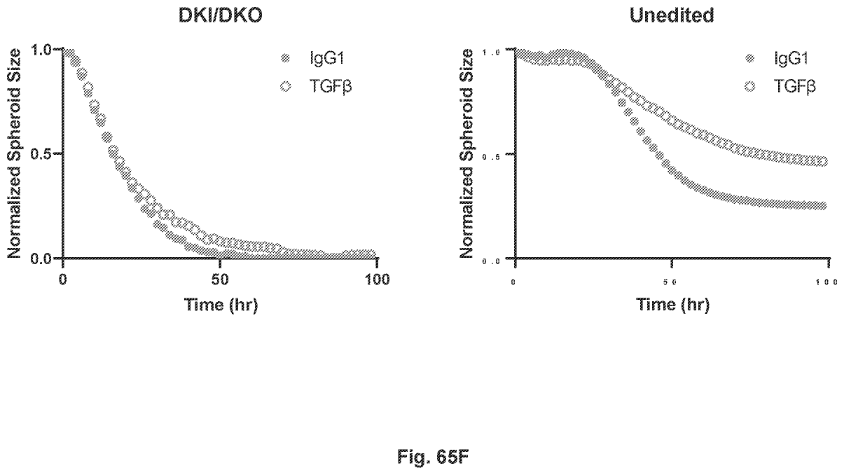

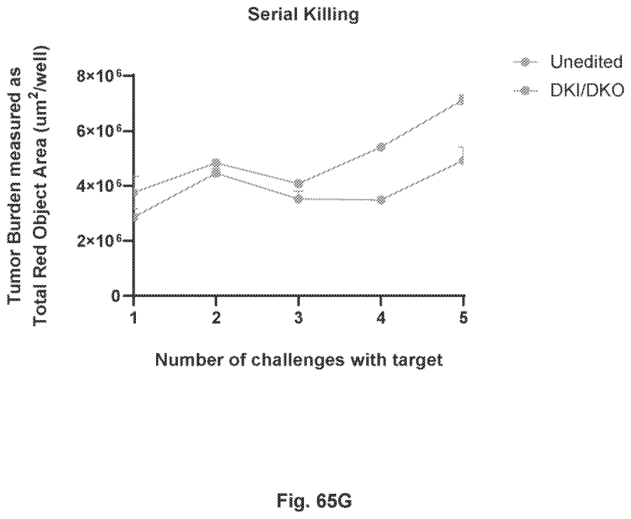

mbIL-15/CD16 double knock-in (DKI)/CISH/TGFβRII double knock-out (DKO) iPSCs were generated using methods described in Examples 14 and 19. In brief, the CISH/TGFβRII DKO was generated using RNPs having an engineered Cas 12a (SEQ ID NO: 62) and a gRNA specific for either CISH or TGFβRII having sequences shown in Table 26. Plasmid PLA1834 was used to generate the mbIL-15/CD16 (CD16^{+/+}/mbIL-15^{+/+}) DKI, as described in Example 14. Following confirmation of the DKI/DKO genotype using standard sequencing methods known in the art, colonies of DKI/DKO iPSCs were propagated and cell populations were then differentiated to iNK cells using a spin embryoid method. As expected. DKI/DKO iNK cells displayed significantly greater CD16 and IL-15Rα expression as compared to unedited (WT) iNK cells (FIG. 65A).

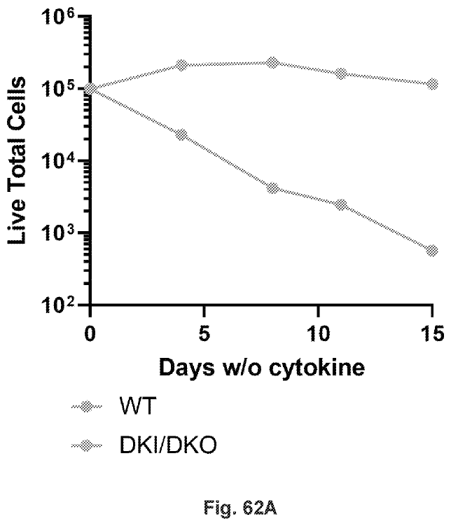

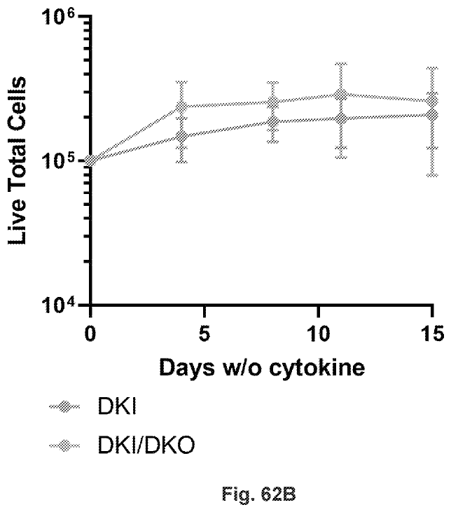

In an in vitro persistence assay, mbIL-15/CD16 (CD16^{+/+}/mbIL-15^{+/+}) DKI/CISH/TGFβRII DKO iNK cells maintained a stable cell number across at least 15 days in the absence of exogenous cytokine support (FIG. 62A). Unedited (WT) iNK cells displayed a substantial decrease in cell number over the same time period. As shown in FIG. 65D. DKI/DKO iNK cells displayed stable viability across at least 16 days without exogenous cytokine support, while WT iNK cells displayed a substantial decrease in culture viability over the same time period. Additionally, the DKI/DKO iNK cells showed comparable total live cell count across at least 15 days without exogenous cytokines as compared to mbIL-15/CD16 (CD16^{+/+}/mbIL-15^{+/+}) DKI iNK cells (FIG. 62B). Thus, mbIL-15/CD16 (CD16^{+/+}/mbIL-15^{+/+}) DKI/CISH/TGFβRII DKO iNK cells demonstrated increased cytokine-independent persistence across at least 15 or 16 days as compared to unedited (WT) iNK cells and similar persistence across this time period as mbIL-15/CD16 (CD16^{+/+}/mbIL-15^{+/+}) DKI iNK cells.

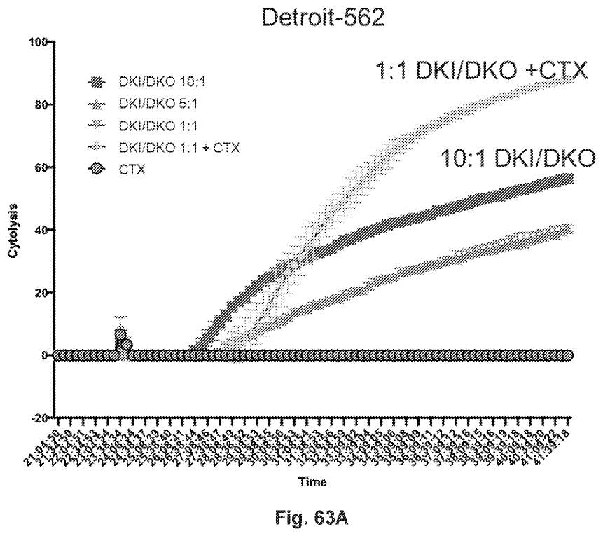

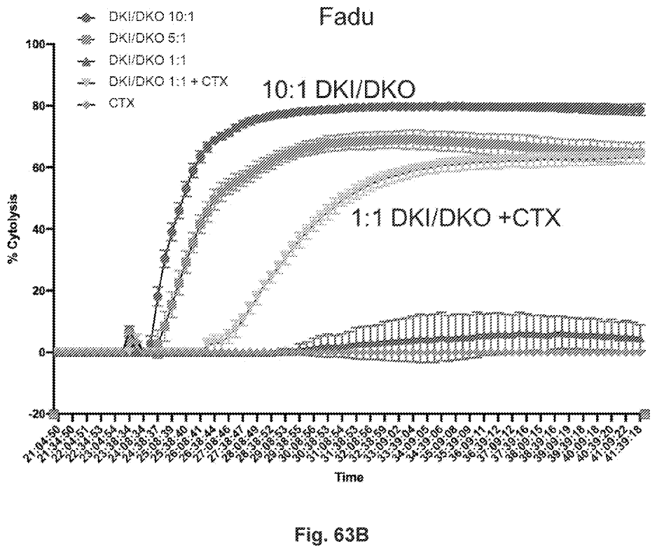

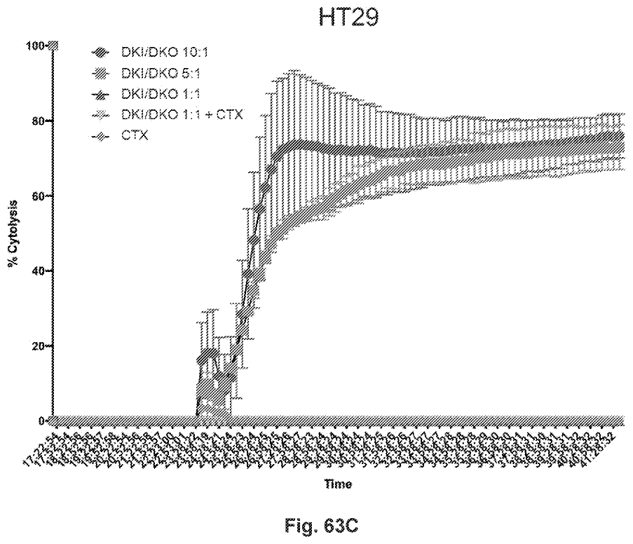

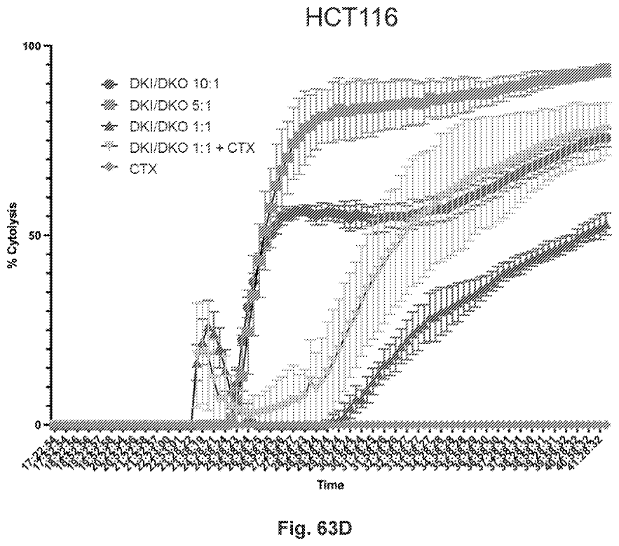

Tumor cell killing ability of the mbIL-15/CD16 (CD16^{+/+}/mbIL-15^{+/+}) DKI/CISH/TGFβRII DKO (DKI/DKO) iNK cells was evaluated in a number of in vitro tumor cell killing assays. Detroit-562 (pharyngeal carcinoma), FaDu (pharyngeal carcinoma), HT-29 (colorectal adenocarcinoma), or HCT116 (colorectal carcinoma) cells were seeded into Xcelligence plates (ACEA #5232376001) at 10,000 cells per well and incubated overnight (˜20 hours). DKI/DKO iNK cells were added at various Effector: Target (E:T) ratios. For the 1:1 E:T condition, 10 μg/mL anti-EGFR antibody cetuximab (CTX) was also included. Cytolysis, as measured by electrical impedance, was assayed according to the manufacturer's (Xcelligence) protocol. Results are shown in FIGS. 63A-D (average±standard deviation: N=3). As depicted, for target Detroit-562, FaDu, HT-29, or HCT116 cells, DKI/DKO iNK cells at 5:1 and 10:1 E:T ratios resulted in significant cytolysis. Additionally, for target HCT117 cells, DKI/DKO iNK cells at a 1:1 E:T ratio also resulted in significant cytolysis. Moreover, for Detroit-562, FaDu, or HCT116 cells, DKI/DKO iNK cells at a 1:1 E:T ratio in combination with cetuximab resulted in significant cytolysis, with the resulting cytolysis greater than the combined effect observed for DKI/DKO iNK cells at a 1:1 E:T ratio alone or cetuximab alone.

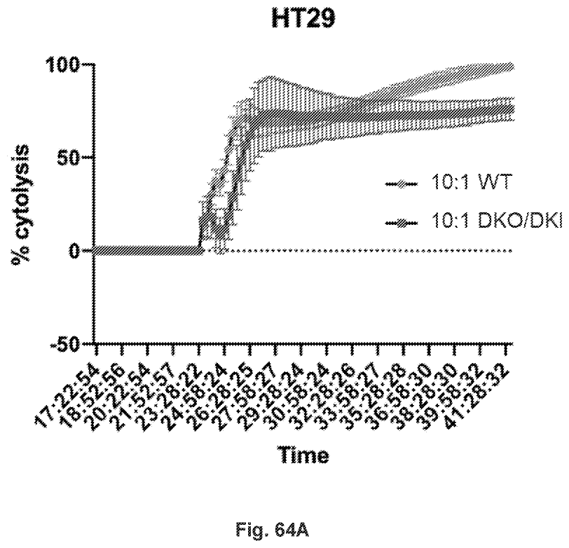

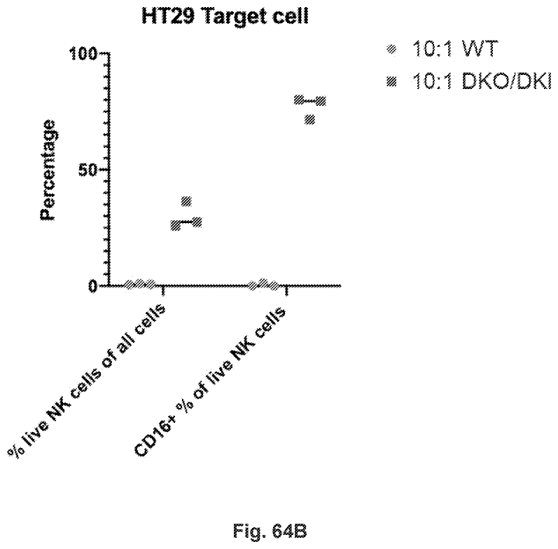

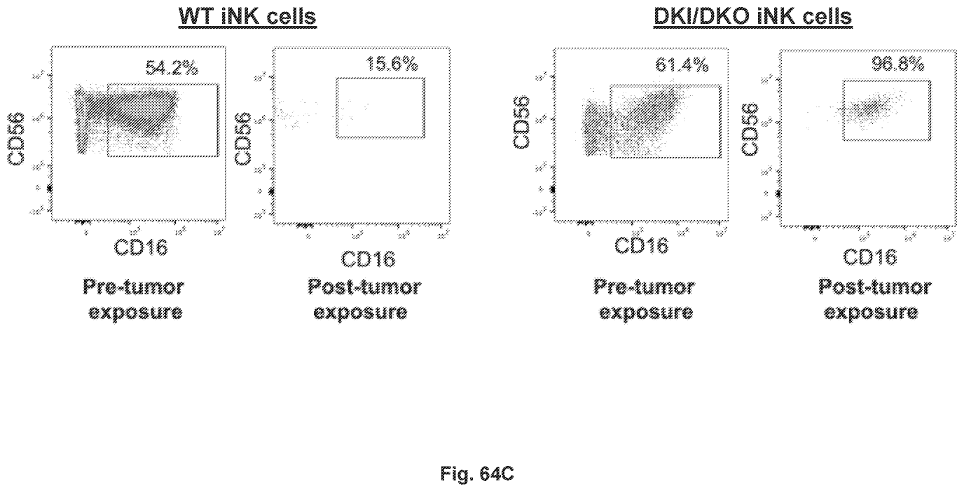

Further tumor cell killing assays were conducted with the mbIL-15/CD16 (CD16^{+/+}/mbIL-15^{+/+}) DKI/CISH/TGFβRII DKO (DKI/DKO) iNK cells. Again, HT-29 (colorectal adenocarcinoma) cells were seeded into Xcelligence plates (ACEA #5232376001) at 10,000 cells per well and incubated overnight. DKI/DKO iNK cells or unedited (WT) iNK cells were then added at a 10:1 E:T ratio. Cytolysis, as measured by electrical impedance, was assayed according to the manufacturer's (Xcelligence) protocol. Results are shown in FIG. 64A (average±standard deviation: N=3). As depicted, both DKI/DKO and WT iNK cells resulted in significant cytolysis. In vitro persistence of mbIL-15/CD16 (CD16^{+/+}/mbIL-15^{+/+}) DKI/CISH/TGFβRII DKO (DKI/DKO) iNK cells and unedited (WT) iNK cells was also examined using HT-29 cells. DKI/DKO iNK cells and WT iNK cells were co-cultured with HT-29 cells for 4 days at a 10:1 E:T ratio. FIG. 64B depicts viability and CD16 expression, as measured by flow cytometry, at day 4. As depicted, WT iNK cells largely did not survive after killing HT-29 cells, whereas DKI/DKO cells persisted. Additionally, whereas surviving WT iNK cells were <1% CD16+, surviving DKI/DKO iNK cells were >80% CD16+. This in vitro persistence assay was repeated at a 1:1 E:T ratio. As shown in FIG. 64C, the DKI/DKO cells again demonstrated greater persistence and maintenance CD16 expression following exposure to tumor (HT-29) cells.

Testing of the mbIL-15/CD16 (CD16^{+/+}/mbIL-15^{+/+}) DKI/CISH/TGFβRII DKO (DKI/DKO) iNK cells in a 3D solid tumor killing assay was also conducted similarly to the depiction in FIG. 20. Briefly, spheroids were formed by seeding 5,000 NucLight Red labeled SK-OV-3 cells in 96 well ultra-low attachment plates. Spheroids were incubated at 37° C. before addition of mbIL-15/CD16 (CD16^{+/+}/mbIL-15^{+/+}) DKI/CISH/TGFβRII DKO (DKI/DKO) iNK cells at various E:T ratios. Spheroids were subsequently imaged every 2 hours using the Incucyte S3 system for up to 4 days. Data were normalized to the red object intensity at time of effector addition. Results are shown in FIG. 65B (N=1; 2 technical replicates per cell line). As depicted, edited DKI/DKO iNK cells were capable of reducing the size of SK-OV-3 spheroids more effectively than unedited (WT) iNK cells. Further 3D tumor killing assays were conducted similarly to the above using DKI/DKO iNK cells or WT iNK cells in combination with 10 μg/ml trastuzumab or IgG (as a control) (FIG. 65C). Potency was determined as IC50, indicating the E:T ratio required to reduce the SK-OV-3 spheroids by 50% after 100 hours of killing. As depicted in FIG. 65C, edited DKI/DKO iNK cells reduced the size of SK-OV-3 spheroids more effectively than unedited (WT) iNK cells, both with and without trastuzumab. The results seen in combination with trastuzumab suggest that DKI/DKO iNK cells mediate enhanced antibody-dependent cellular cytotoxicity (ADCC) relative to WT iNK cells.

A phosphoflow cytometry assay was performed to determine the phosphorylated state of SMAD2/3 (pSMAD2/3) in mbIL-15/CD16 (CD16^{+/+}/mbIL-15^{+/+}) DKI/CISH/TGFβRII DKO (DKI/DKO) iNK cells. Briefly, DKI/DKO iNKs were plated the day before in a cytokine starved condition. The next day, the cells were stimulated with 10 ng/ml of TGFβ for a set length of time (e.g., 0)-60) minutes). The cells were fixed immediately at the end of the time point and stained. The cells were processed on a NovoCyte Quanteon and the data was analyzed in FlowJo. Results are shown in FIG. 65E (data represents 1 independent experiment). SMAD2/3 phosphorylation in DKI/DKO iNK cells was unchanged in the presence of TGFβ, while TGFβ increased SMAD2/3 phosphorylation in unedited (WT) iNK cells. Additionally, a 3D solid tumor cell killing assay was performed, similarly as described above, using mbIL-15/CD16 (CD16^{+/+}/mbIL-15^{+/+}) DKI/CISH/TGFβRII DKO (DKI/DKO) iNK cells at an E:T ratio of 31.6 and with or without 10 ng/ml TGFβ. As shown in FIG. 65F, DKI/DKO iNK cells were capable of reducing the size of SK-OV-3 spheroids more effectively than unedited iNK control cells after 100 hrs of killing (data represents 1 independent experiment). Moreover, DKI/DKO iNK cell activity was unaffected by the presence of exogenous TGFβ, in contrast to unedited (WT) iNKs. mbIL-15/CD16 (CD16^{+/+}/mbIL-15^{+/+}) DKI/CISH/TGFβRII DKO (DKI/DKO) iNK cells were also pushed to kill tumor targets repeatedly over a multiday period in an in vitro serial killing assay. At day 0 of the assay, 10×10³ Nalm6 tumor cells (a B cell leukemia cell line) and 2×10⁵ DKI/DKO iNKs were plated in each well of a 96-well plate in the presence of TGFβ (10 ng/ml). At 48 hour intervals, a bolus of 5×10³ Nalm6 tumor cells was added to re-challenge the DKI/DKO iNK population. Results are shown in FIG. 65G (N=1; 3 technical replicates per cell line: error bars=standard deviation). As depicted, the DKI/DKO iNK cells exhibited continued killing of Nalm6 tumor cells after multiple challenges with Nalm6 tumor cells, even in the presence of TGFβ. In contrast, unedited (WT) iNK cells were limited in their serial killing effect. Taken all together, these data suggest that DKI/DKO iNKs have enhanced resistance to TGFβ-mediated immunosuppression.

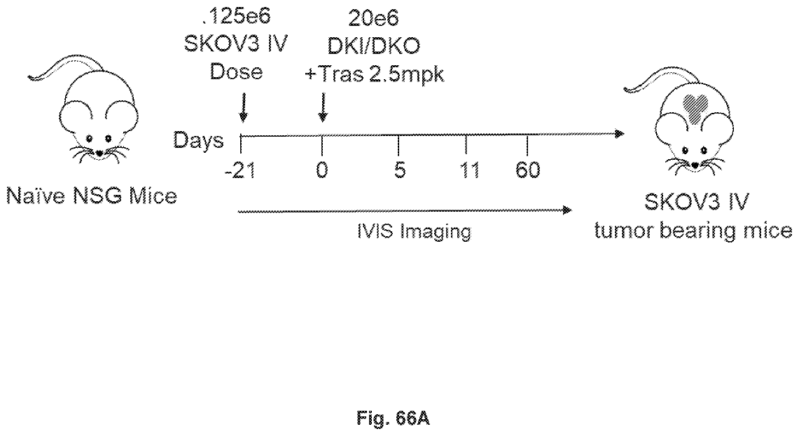

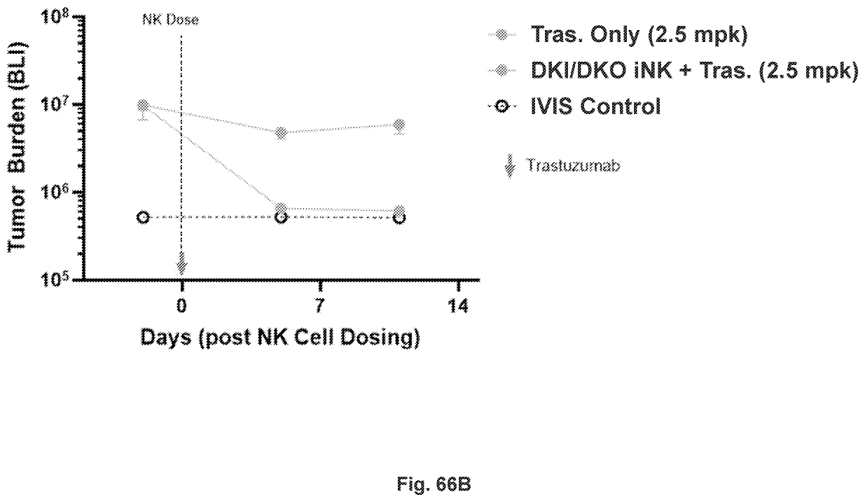

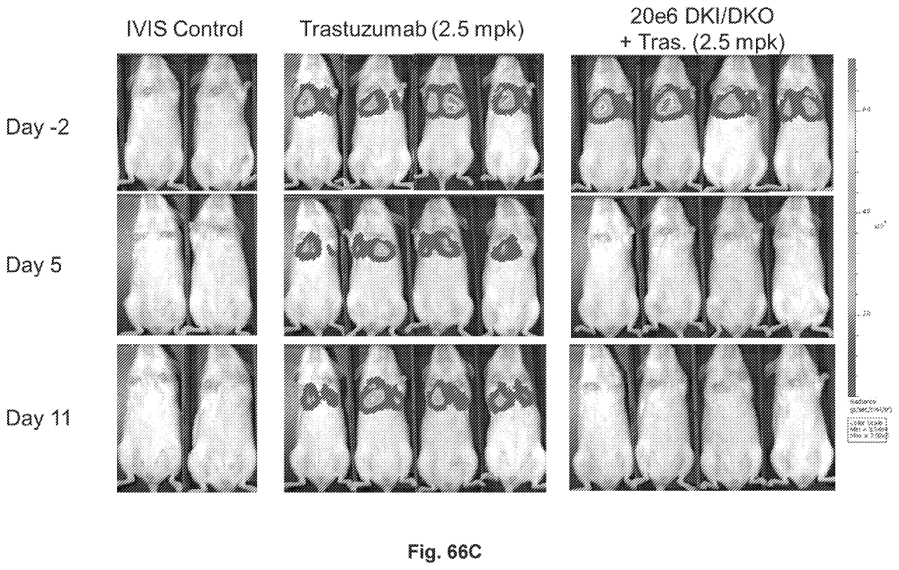

Testing of the mbIL-15/CD16 (CD16^{+/+}/mbIL-15^{+/+}) DKI/CISH/TGFβRII DKO (DKI/DKO) iNK cells in an in vivo mouse model was conducted as depicted in FIG. 66A. Mice were intravenously (IV) inoculated with 0.125×10⁶ SKOV3-luc cells. Following 19 days to allow for establishment of the tumors, mice were imaged using an in vivo imaging system (IVIS) to establish pre-treatment (day −2) tumor burden and then randomized into treatment groups. On day 0, mice were injected intravenously with (i) 2.5 mpk trastuzumab, or (ii) 20×10⁶ DKI/DKO iNK cells and 2.5 mpk trastuzumab. Following day 0), the mice were imaged using an IVIS to assess tumor burden over time. The tumor burden over time as measured by bioluminescent imaging (BLI) via IVIS is depicted in FIGS. 66B-66C. Tumor burden is displayed in FIG. 66B, and representative bioluminescent imaging of the mice at various time points is displayed in FIG. 66C. As depicted, mice treated with DKI/DKO iNK cells in combination with trastuzumab exhibited greater tumor reduction than mice treated with trastuzumab alone. Mice treated with DKI/DKO iNK cells in combination with trastuzumab exhibited significant tumor reduction after just 5 days. Furthermore, treatment with the DKI/DKO iNK cells in combination with trastuzumab led to complete tumor clearance in all of the animals in the treatment group at day 5.

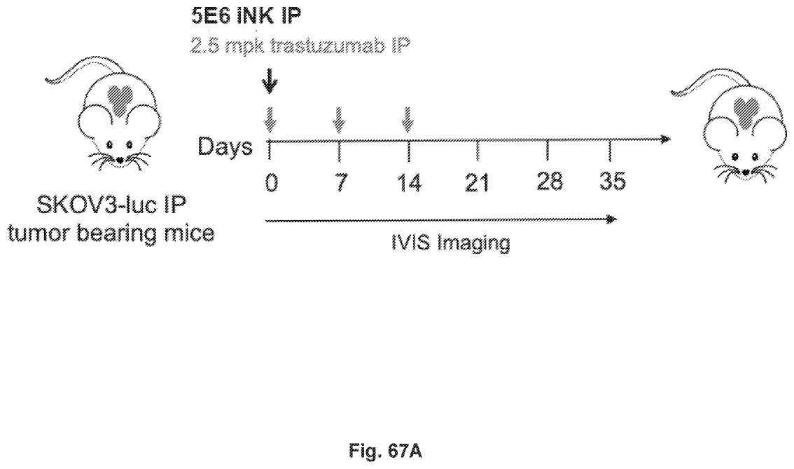

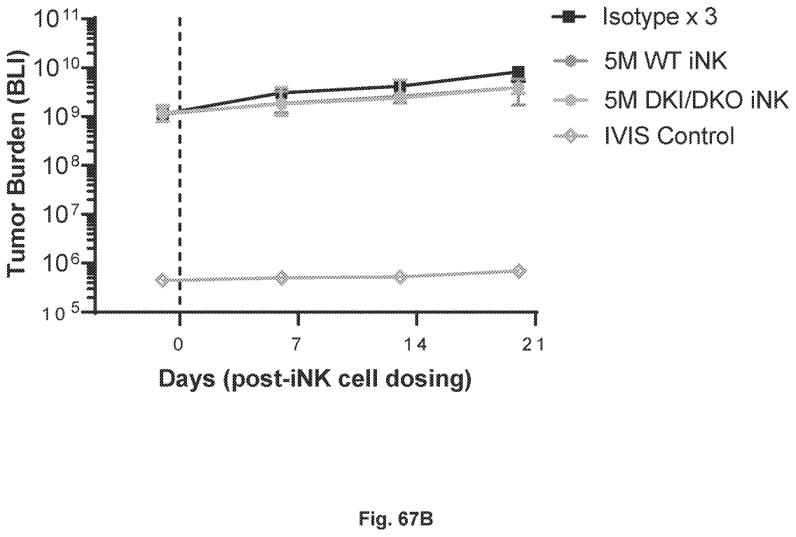

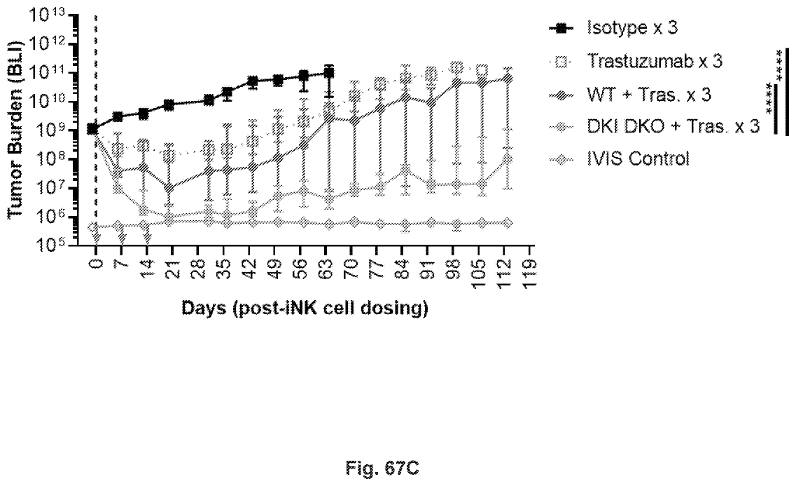

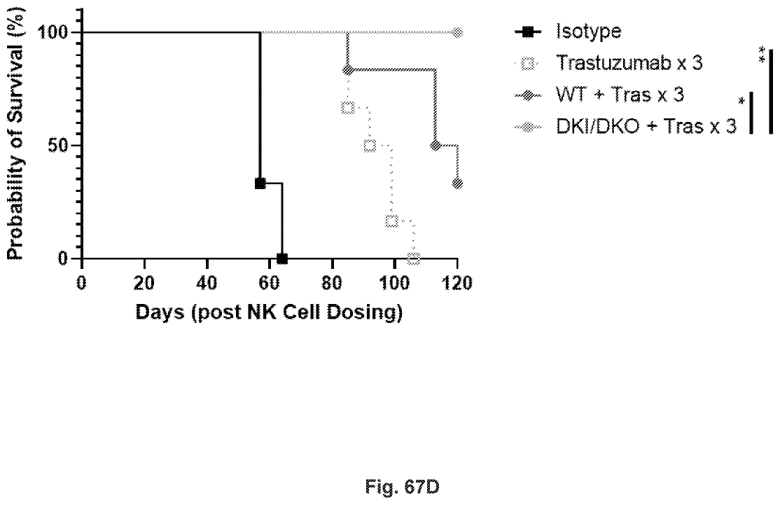

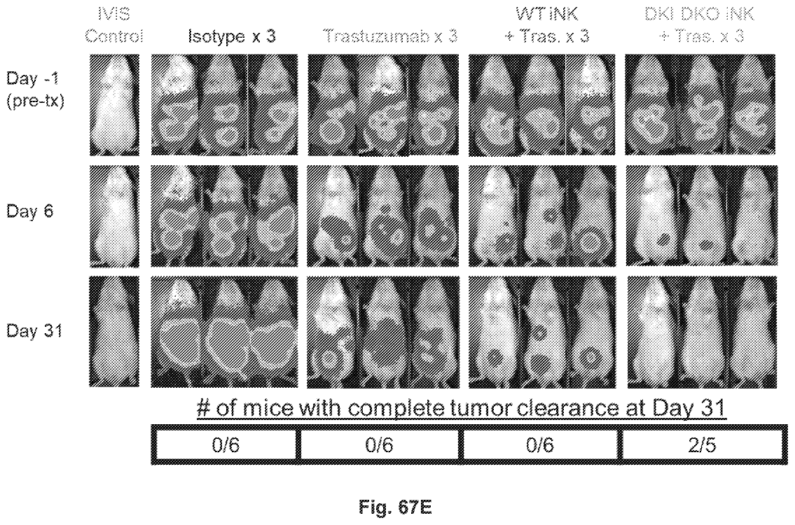

Additional testing of the mbIL-15/CD16 (CD16^{+/+}/mbIL-15^{+/+}) DKI/CISH/TGFβRII DKO (DKI/DKO) iNK cells in an in vivo mouse model was conducted as depicted in FIG. 67A. Mice were intraperitoneally inoculated with 0.25×10⁶ SKOV3-luc cells. Following 4 days to allow for establishment of the tumors, mice were imaged using an in vivo imaging system (IVIS) to establish pre-treatment (day −1) tumor burden and then randomized into treatment groups. After 1 additional day (on day 0), mice were injected intraperitoneally (IP) with 5×10⁶ unedited (WT) iNK cells, 5×10⁶ DKI/DKO iNK cells, or no iNK cells for trastuzumab-alone or the isotype control. Some treatment groups (“Trastuzumab×3” or “+Tras.×3”) received IP injections of 2.5 mpk trastuzumab on days 0, 7, and 14. Following day 0), the mice were imaged weekly using an IVIS to assess tumor burden over time. The tumor burden over time as measured by bioluminescent imaging (BLI) via IVIS is depicted in FIGS. 67B-C. Representative bioluminescent imaging of the mice at various time points is displayed in FIG. 67E. As seen in FIG. 67B, treatment with the unedited (WT) iNK cells or the DKI/DKO iNK cells alone did not lead to tumor reduction in vivo. However, mice treated with iNK cells in combination with trastuzumab exhibited greater tumor reduction than mice treated with trastuzumab alone (FIG. 67C). Mice dosed with mbIL-15/CD16 (CD16^{+/+}/mbIL-15^{+/+}) DKI/CISH/TGFβRII DKO (DKI/DKO) iNK cells in combination with trastuzumab (DKI/DKO+Tras×3) also had significantly prolonged survival compared to mice dosed with unedited (WT) iNK cells in combination with trastuzumab (WT+Tras×3) or trastuzumab alone (FIG. 67D). Moreover, the reduction in tumor burden was greater in mice treated with DKI/DKO iNK cells than in mice treated with unedited (WT) iNK cells. Mice treated with DKI/DKO iNK cells in combination with trastuzumab exhibited significant tumor reduction after just 6 days (FIG. 67E). Furthermore, as shown in FIG. 67E, treatment with the DKI/DKO iNK cells in combination with trastuzumab led to complete tumor clearance in two (40%) of the animals in the treatment group at day 31 post-introduction of the NK cells. These results confirm that mbIL-15/CD16 (CD16^{+/+}/mbIL-15^{+/+}) DKI/CISH/TGFβRII DKO iNK cells readily kill tumor cells in vivo and demonstrate that mbIL-15/CD16 (CD16^{+/+}/mbIL-15^{+/+}) DKI/CISH/TGFβRII DKO iNK cells in combination with trastuzumab produces greater in vivo tumor reduction than treatment with either trastuzumab alone or with unedited (WT) iNK cells in combination with trastuzumab.

### EQUIVALENTS

It is to be understood that while the disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

We claim:

1. A Natural Killer (NK) cell comprising:
(a) one or more genomic edits that results in loss of function of one or more genes, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof; and
(b) a genome comprising a first exogenous coding sequence for FcγRIII (CD16) or variant thereof and a second exogenous coding sequence for a membrane bound interleukin 15 (mbIL-15), wherein the first exogenous coding sequence and second exogenous coding sequence are in frame with and downstream (3′) of a coding sequence of an essential gene, and wherein at least part of the essential gene comprises an exogenous coding sequence.

2. The NK cell of claim 1, wherein the genome comprises:
(i) the first exogenous coding sequence and the second exogenous coding sequence at a first allele of the essential gene; and
(ii) the first exogenous coding sequence and the second exogenous coding sequence at a second allele of the essential gene.

3. The NK cell of claim 1 or 2, wherein the first exogenous coding sequence is upstream (5′) of the second exogenous coding sequence.

4. The NK cell of claim 3, wherein the genome comprises:
(i) a first regulatory element between the coding sequence of the essential gene and the first exogenous coding sequence; and
(ii) a second regulatory element between the first exogenous coding sequence and the second exogenous coding sequence.

5. The NK cell of claim 4, wherein the first regulatory element is an IRES or 2A element and the second regulatory element is an IRES or 2A element.

6. The NK cell of any one of claims 3-5, wherein the genome comprises a polyadenylation sequence downstream (3′) of the second exogenous coding sequence.

7. The NK cell of claim 6, wherein the genome comprises a 3′ untranslated region (UTR) sequence downstream (3′) of the second exogenous coding sequence and upstream (5′) of the polyadenylation sequence.

8. The NK cell of claim 1 or 2, wherein the second exogenous coding sequence is upstream (5′) of the first exogenous coding sequence.

9. The NK cell of claim 8, wherein the genome comprises:
(i) a first regulatory element between the coding sequence of the essential gene and the second exogenous coding sequence; and
(ii) a second regulatory element between the second exogenous coding sequence and the first exogenous coding sequence.

10. The NK cell of claim 9, wherein the first regulatory element is an IRES or 2A element and the second regulatory element is an IRES or 2A element.

11. The NK cell of any one of claims 8-10, wherein the genome comprises a polyadenylation sequence downstream (3′) of the first exogenous coding sequence.

12. The NK cell of claim 11, wherein the genome comprises a 3′ untranslated region (UTR) sequence downstream (3′) of the first exogenous coding sequence and upstream (5′) of the polyadenylation sequence.

13. The NK cell of any one of the preceding claims, wherein the first exogenous coding sequence is or comprises SEQ ID NO: 166.

14. The NK cell of any one of the preceding claims, wherein the second exogenous coding sequence is or comprises SEQ ID NO: 172.

15. The NK cell of any one of the preceding claims, wherein the CD16 is or comprises the amino acid sequence of SEQ ID NO: 184.

16. The NK cell of any one of the preceding claims, wherein the mbIL-15 comprises an IL-15, a linker, a sushi domain, and an IL-15Rα.

17. The NK cell of claim 16, wherein the mbIL-15 is or comprises the amino acid sequence of SEQ ID NO: 190.

18. The NK cell of any one of the preceding claims, wherein the NK cell is an induced pluripotent stem cell (iPSC)-derived NK (INK) cell.

19. The NK cell of any one of the preceding claims, wherein the essential gene encodes a gene product that is required for survival and/or proliferation of the cell.

20. The NK cell of any one of the preceding claims, wherein the essential gene is a housekeeping gene, e.g., a gene listed in Table 3.

21. The NK cell of any one of the preceding claims, wherein the essential gene encodes glyceraldehyde 3-phosphate dehydrogenase (GAPDH).

22. The NK cell of any one of the preceding claims, wherein the NK cell comprises:
(i) a genomic edit that results in loss of function of CISH; and
(ii) a genomic edit that results in loss of function of TGFβRII.

23. The NK cell of any one of the preceding claims, for use as a medicament.

24. The NK cell of any one of the preceding claims, for use in the treatment of a disease, disorder, or condition, e.g., a tumor and/or a cancer.

25. A progeny or daughter cell of the NK cell of any one of claims 1-24.

26. A population of NK cells comprising the NK cell of any one of claims 1-24.

27. The population of NK cells of claim 26, characterized in that, when contacted with tumor cells, a level of killing of tumor cells by the NK cells is increased relative to a reference level of killing of tumor cells by a reference population of NK cells.

28. The population of NK cells of claim 26 or 27, characterized in that, when contacted with tumor cells and an antibody, a level of antibody-dependent cellular cytotoxicity (ADCC) induced by the NK cells is increased relative to a reference level of ADCC induced by a reference population of NK cells.

29. The population of NK cells of any one of claims 26-28, wherein a level of persistence of the population of NK cells is increased relative to a reference level of persistence of a reference population of NK cells.

30. The population of NK cells of claim 29, wherein the level of persistence is measured following contacting with tumor cells.

31. The population of NK cells of any one of claims 26-30, wherein the reference population of NK cells does not comprise NK cells comprising a genome comprising the first exogenous coding sequence and the second exogenous coding sequence.

32. The population of NK cells of claim 31, wherein the reference population of NK cell does not comprise NK cells comprising a genomic edit that results in loss of function of TGFβRII and a genomic edit that results in loss of function of CISH.

33. A pharmaceutical composition comprising the NK cell, the progeny or daughter cell, or the population of NK cells of any one of claims 1-32.

34. The pharmaceutical composition of claim 33, comprising a pharmaceutically acceptable carrier.

35. A method of treating a condition, disorder, and/or disease, comprising administering to a subject suffering therefrom the NK cell, the progeny or daughter cell, or the population of NK cells of any one of claims 1-32.

36. The method of claim 35, wherein the subject is suffering from a tumor, e.g., a solid tumor.

37. The method of claim 35, wherein the subject is suffering from a cancer.

38. A method, comprising administering to a subject the NK cell, the progeny or daughter cell, or the population of NK cells of any one of claims 1-32.

39. A method of treating a condition, disorder, and/or disease, comprising administering to a subject suffering therefrom the pharmaceutical composition of claim 33 or 34.

40. The method of claim 39, wherein the subject is suffering from a tumor, e.g., a solid tumor.

41. The method of claim 39, wherein the subject is suffering from a cancer.

42. A method, comprising administering to a subject the pharmaceutical composition of claim 33 or 34.

43. The method of any one of claims 35-42, wherein the NK cell, the progeny or daughter cell, or the population of NK cells is allogenic to the subject.

44. The method of any one of claims 35-42, wherein the NK cell, the progeny or daughter cell, or the population of NK cells is autologous to the subject.

45. The method of any one of claims 35-44, further comprising administering an antibody to the subject.

46. The method of claim 45, wherein the antibody is trastuzumab, rituximab, or cetuximab.

47. The method of any one of claims 35-46, wherein the subject is a human.

48. A method of increasing tumor killing ability of a NK cell, the method comprising:
(a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and
(b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof;
thereby increasing a level of tumor killing activity of the NK cell relative to a reference level of tumor killing activity of a reference NK cell.

49. A method of increasing antibody-dependent cellular cytotoxicity (ADCC) induced by a NK cell, the method comprising:
(a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and
(b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof;
thereby increasing a level of ADCC induced by the NK cell relative to a reference level of ADCC induced by a reference NK cell.

50. A method of increasing persistence of a NK cell, the method comprising:
(a) knocking-into the genome of the NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and
(b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof;
thereby increasing a level of persistence of the NK cell relative to a reference level of persistence of a reference NK cell.

51. The method of claim 50, wherein the level of persistence is measured following contacting the NK cell with tumor cells.

52. The method of any one of claims 48-51, wherein the reference NK cell does not comprise a genome comprising the first exogenous coding sequence and the second exogenous coding sequence.

53. The method of any one of claims 48-52, wherein the reference NK cell does not comprise a genomic edit that results in loss of function of TGFβRII and a genomic edit that results in loss of function of CISH.

54. A method of manufacturing a genetically modified NK cell, the method comprising:
(a) knocking-into the genome of an NK cell a first exogenous coding sequence for FcγRIII (CD16) or variant thereof and a second exogenous coding sequence for a membrane bound interleukin 15 (mbIL-15), wherein the first exogenous coding sequence and the second exogenous coding sequence are knocked-in in frame and downstream (3′) of an essential gene; and
(b) knocking-out one or more genes of the NK cell, wherein the one or more genes encode adenosine A2a receptor (ADORA2A), β-2 microglobulin (B2M), class II major histocompatibility complex transactivator (CIITA), cytokine inducible SH2 containing protein (CISH), two or more human leukocyte antigen (HLA) class II histocompatibility antigen alpha chain genes, two or more HLA class II histocompatibility antigen beta chain genes, natural killer group 2 member A receptor (NKG2A), programmed cell death protein 1 (PD-1), T cell immunoreceptor with Ig and ITIM domains (TIGIT), an agonist of the TGF beta signaling pathway (e.g., transforming growth factor beta receptor II (TGFβRII)), or any combination of two or more thereof.

55. The method of any one of claims 48-54, wherein knocking-in comprises contacting the NK cell with:
(i) a nuclease that causes a break within an endogenous coding sequence of the essential gene, and
(ii) a donor template that comprises a knock-in cassette comprising the first exogenous coding sequence and the second exogenous coding sequence in frame with and downstream (3′) of an exogenous coding sequence or partial coding sequence of the essential gene, wherein the knock-in cassette is integrated into the genome of the cell by homology-directed repair (HDR) of the break.

56. The method of claim 55, wherein the nuclease is a CRISPR/Cas nuclease and knocking-in further comprises contacting the NK cell with a guide molecule for the CRISPR/Cas nuclease.

57. The method of any one of claims 48-56, wherein knocking-out comprises contacting the NK cell with one or more nucleases that cause a break within an endogenous coding sequence of the one or more genes.

58. The method of claim 57, wherein the one or more nucleases are CRISPR/Cas nucleases and knocking-out further comprises contacting the NK cell with one or more guide molecules for the CRISPR/Cas nuclease.

59. The method of any one of claims 48-58, wherein the NK cell is an induced pluripotent stem cell (iPSC)-derived NK (iNK) cell.

60. The method of any one of claims 48-59, wherein the essential gene encodes a gene product that is required for survival and/or proliferation of the NK cell.

61. The method of any one of claims 48-59, wherein the essential gene is a housekeeping gene, e.g., a gene listed in Table 3.

62. The method of any one of claims 48-60, wherein the essential gene encodes glyceraldehyde 3-phosphate dehydrogenase (GAPDH).

63. The method of any one of claims 48-62, comprising knocking-out a gene encoding CISH and knocking-out a gene encoding TGFβRII.
